(19) United States
(12) Patent Application Publication
(10) Pub. No.: US 2024/0042015 A1
(43) Pub. Date: Feb. 8, 2024

(54) CIRCULAR RNA COMPOSITIONS AND METHODS

(71) Applicant: Orna Therapeutics, Inc., Cambridge, MA (US)

(72) Inventors: Alexander Wesselhoeft, Cambridge, MA (US); Thomas Barnes, Cambridge, MA (US); Brian Goodman, Cambridge, MA (US); Greg Motz, Cambridge, MA (US); Amy Becker, Cambridge, MA (US); Allen T. Horhota, Cambridge, MA (US)

(21) Appl. No.: 17/999,378

(22) PCT Filed: May 19, 2021
(86) PCT No.: PCT/US2021/033276

§ 371 Date: Nov. 18, 2022

Related U.S. Application Data
(60) Provisional application No. 63/027,292, filed on May 19, 2020.

(51) Int. Cl. A61K 39/295; A61P 37/04

(52) U.S. Cl. CPC A61K 39/295; A61P 37/04; A61K 2039/55555

(57) ABSTRACT

Circular RNA, along with related compositions and methods are described herein. In some embodiments, the inventive circular RNA comprises group I intron fragments, spacers, an IRES, duplex forming regions, and an expression sequence. In some embodiments, the expression sequence encodes an antigen. In some embodiments, circular RNA of the invention has improved expression, functional stability, immunogenicity, ease of manufacturing, and/or half-life when compared to linear RNA. In some embodiments, inventive methods and constructs result in improved circularization efficiency, splicing efficiency, and/or purity when compared to existing RNA circularization approaches.

### CROSS REFERENCE TO RELATED APPLICATIONS

This application claims the benefit of, and priority to, U.S. Provisional Application No. 63/027,292, filed on May 19, 2020, the contents of which are hereby incorporated by reference in their entirety for all purposes.

### BACKGROUND

Conventional gene therapy involves the use of DNA for insertion of desired genetic information into host cells. The DNA introduced into the cell is usually integrated to a certain extent into the genome of one or more transfected cells, allowing for long-lasting action of the introduced genetic material in the host. While there may be substantial benefits to such sustained action, integration of exogenous DNA into a host genome may also have many deleterious effects. For example, it is possible that the introduced DNA will be inserted into an intact gene, resulting in a mutation which impedes or even totally eliminates the function of the endogenous gene. Thus, gene therapy with DNA may result in the impairment of a vital genetic function in the treated host, such as e.g., elimination or deleteriously reduced production of an essential enzyme or interruption of a gene critical for the regulation of cell growth, resulting in unregulated or cancerous cell proliferation. In addition, with conventional DNA based gene therapy it is necessary for effective expression of the desired gene product to include a strong promoter sequence, which again may lead to undesirable changes in the regulation of normal gene expression in the cell. It is also possible that the DNA based genetic material will result in the induction of undesired anti-DNA antibodies, which in turn, may trigger a possibly fatal immune response. Gene therapy approaches using viral vectors can also result in an adverse immune response. In some circumstances, the viral vector may even integrate into the host genome. In addition, production of clinical grade viral vectors is also expensive and time consuming. Targeting delivery of the introduced genetic material using viral vectors can also be difficult to control. Thus, while DNA based gene therapy has been evaluated for delivery of secreted proteins using viral vectors (U.S. Pat. No. 6,066,626; U.S. Publication No. US2004/0110709), these approaches may be limited for these various reasons.

In contrast to DNA, the use of RNA as a gene therapy agent is substantially safer because RNA does not involve the risk of being stably integrated into the genome of the transfected cell, thus eliminating the concern that the introduced genetic material will disrupt the normal functioning of an essential gene, or cause a mutation that results in deleterious or oncogenic effects, and extraneous promoter sequences are not required for effective translation of the encoded protein, again avoiding possible deleterious side effects. In addition, it is not necessary for mRNA to enter the nucleus to perform its function, while DNA must overcome this major barrier.

Circular RNA is useful in the design and production of stable forms of RNA. The circularization of an RNA molecule provides an advantage to the study of RNA structure and function, especially in the case of molecules that are prone to folding in an inactive conformation (Wang and Ruffner, 1998). Circular RNA can also be particularly interesting and useful for in vivo applications, especially in the research area of RNA-based control of gene expression and therapeutics, including protein replacement therapy and vaccination.

Prior to this invention, there were three main techniques for making circularized RNA in vitro: the splint-mediated method, the permuted intron-exon method, and the RNA ligase-mediated method. However, the existing methodologies are limited by the size of RNA that can be circularized, thus limiting their therapeutic application.

### SUMMARY

Circular RNA, along with related compositions and methods are described herein. In some embodiments, the inventive circular RNA comprises group I intron fragments, spacers, an IRES, duplex forming regions, and an expression sequence. In some embodiments, the expression sequence encodes one or more antigens. In certain embodiments, the expression sequence is replaced with a non-coding sequence. In some embodiments, circular RNA of the invention has improved expression, functional stability, ease of manufacturing, and/or half-life when compared to linear RNA. In some embodiments, circular RNA of the invention has reduced immunogenicity. In some embodiments, inventive methods and constructs result in improved circularization efficiency, splicing efficiency, and/or purity when compared to existing RNA circularization approaches.

In an aspect, provided herein is a circular RNA polynucleotide comprising, in the following order, a. a 3′ group I intron fragment, b. an Internal Ribosome Entry Site (IRES), c. an expression sequence encoding one or more antigens, adjuvants, antigen-like or adjuvant-like polypeptides, or fragments thereof, and d. a 5′ group I intron fragment. In some embodiments, a 3′ group I intron fragment includes a 3′ group I intron splice site dinucleotide. In some embodiments, a 5′ group I intron fragment includes a 5′ group I intron splice site dinucleotide.

In an aspect, provided herein is a circular RNA polynucleotide comprising, in the following order, a. a 3′ group I intron fragment, b. an Internal Ribosome Entry Site (IRES), c. a non-coding expression sequence, and d. a 5′ group I intron fragment.

In an aspect, provided herein is a circular RNA polynucleotide produced from transcription of a vector comprising, in the following order, a. a 5′ duplex forming region, b. a 3′ group I intron fragment, c. an Internal Ribosome Entry Site (IRES), d. an expression sequence encoding for one or more antigens, adjuvants, antigen-like or adjuvant-like polypeptides, or fragments thereof, e. a 5′ group I intron fragment, and f. a 3′ duplex forming region.

In an aspect, provided herein is a circular RNA polynucleotide produced from transcription of a vector comprising, in the following order, a. a 5′ duplex forming region, b. a 3′ group I intron fragment, c. an Internal Ribosome Entry Site (IRES), d. a non-coding expression sequence, e. a 5′ group I intron fragment, and f. a 3′ duplex forming region.

In some embodiments, the vector further comprises a triphosphorylated 5′ terminus. In some embodiments, the vector further comprises a monophosorylated 5′ terminus.

In some embodiments, the circular RNA polynucleotide comprises a first spacer between the 5′ duplex forming region and the 3′ group I intron fragment, and a second spacer between the 5′ group I intron fragment and the 3′ duplex forming region. In some embodiments, the first and second spacers each have a length of about 10 to about 60 nucleotides. In some embodiments, the first and second duplex forming regions each have a length of about 9 to about 19 nucleotides. In some other embodiments, the first and second duplex forming regions each have a length of about 30 nucleotides.

In some embodiments, the IRES has a sequence of an IRES from Taura syndrome virus, Triatoma virus, Theiler's encephalomyelitis virus, Simian Virus 40, Solenopsis invicta virus 1, Rhopalosiphum padi virus, Reticuloendotheliosis virus, Human poliovirus 1, Plautia stali intestine virus, Kashmir bee virus, Human rhinovirus 2, Homalodisca coagulata virus-1, Human Immunodeficiency Virus type 1, Homalodisca coagulata virus-1, Himetobi P virus, Hepatitis C virus, Hepatitis A virus, Hepatitis GB virus, Foot and mouth disease virus, Human enterovirus 71, Equine rhinitis virus, Ectropis obliqua picorna-like virus, Encephalomyocarditis virus, Drosophila C Virus, Human coxsackievirus B3, Crucifer tobamovirus, Cricket paralysis virus, Bovine viral diarrhea virus 1, Black Queen Cell Virus, Aphid lethal paralysis virus, Avian encephalomyelitis virus, Acute bee paralysis virus, Hibiscus chlorotic ringspot virus, Classical swine fever virus, Human FGF2, Human SFTPA 1, Human AML1/RUNX1, Drosophila antennapedia, Human AQP4, Human ATIR, Human BAG-1, Human BCL2, Human BiP, Human c-IAP1, Human c-myc, Human eIF4G, Mouse NDST4L, Human LEF1, Mouse HIF1 alpha, Human n.myc, Mouse Gtx, Human p27kipl, Human PDGF2/c-sis, Human p53, Human Pim-1, Mouse Rbm3, Drosophila reaper, Canine Scamper, Drosophila Ubx, Human UNR, Mouse UtrA, Human VEGF-A, Human XIAP, Drosophila hairless, S. cerevisiae TFIID, S. cerevisiae YAP1, tobacco etch virus, turnip crinkle virus, EMCV-A, EMCV-B, EMCV-Bf, EMCV-Cf, EMCV pEC9, Picobirnavirus, HCV QC64, Human Cosavirus E/D, Human Cosavirus F, Human Cosavirus JMY, Rhinovirus NAT001, HRV14, HRV89, HRVC-02, HRV-A21, Salivirus A SH1, Salivirus FHB, Salivirus NG-J1, Human Parechovirus 1, Crohivirus B, Yc-3, Rosavirus M-7, Shanbavirus A, Pasivirus A, Pasivirus A 2, Echovirus E14, Human Parechovirus 5, Aichi Virus, Hepatitis A Virus HA16, Phopivirus, CVA10, Enterovirus C, Enterovirus D, Enterovirus J, Human Pegivirus 2, GBV-C GT110, GBV-C K1737, GBV-C Iowa, Pegivirus A 1220, Pasivirus A 3, Sapelovirus, Rosavirus B, Bakunsa Virus, Tremovirus A, Swine Pasivirus 1, PLV-CHN, Pasivirus A, Sicinivirus, Hepacivirus K, Hepacivirus A, BVDV1, Border Disease Virus, BVDV2, CSFV-PK15C, SF573 Dicistrovirus, Hubei Picorna-like Virus, CRPV, Salivirus A BN5, Salivirus A BN2, Salivirus A 02394, Salivirus A GUT, Salivirus A CH, Salivirus A SZ1, Salivirus FHB, CVB3, CVB1, Echovirus 7, CVB5, EVA71, CVA3, CVA12, EV24 or an aptamer to eIF4G.

In some embodiments, the circular RNA polynucleotide consists of natural nucleotides. In some embodiments, the expression sequence is codon-optimized. In some embodiments, the circular RNA polynucleotide is optimized to lack at least one microRNA binding site present in an equivalent pre-optimized polynucleotide. In some embodiments, the circular RNA polynucleotide is optimized to lack at least one endonuclease susceptible site present in an equivalent pre-optimized polynucleotide. In some embodiments, the circular RNA polynucleotide is optimized to lack at least one RNA-editing susceptible site present in an equivalent pre-optimized polynucleotide.

In some embodiments, the circular RNA polynucleotide is from about 100 nucleotides to about 10 kilobases in length.

In some embodiments, the circular RNA polynucleotide has an in vivo duration of therapeutic effect in humans of at least about 20 hours. In some embodiments, the circular RNA polynucleotide has a functional half-life of at least about 20 hours. In some embodiments, the circular RNA polynucleotide has a duration of therapeutic effect in a human cell greater than or equal to that of an equivalent linear RNA polynucleotide comprising the same expression sequence. In some embodiments, the circular RNA polynucleotide has a functional half-life in a human cell greater than or equal to that of an equivalent linear RNA polynucleotide comprising the same expression sequence. In some embodiments, the circular RNA polynucleotide has a in vivo duration of therapeutic effect in humans greater than that of an equivalent linear RNA polynucleotide having the same expression sequence. In some embodiments, the circular RNA polynucleotide has an in vivo functional half-life in humans greater than that of an equivalent linear RNA polynucleotide having the same expression sequence.

In some embodiments, the adjuvant or adjuvant-like polypeptide is selected from the group comprising toll-like receptor ligand, cytokine, FLt3-ligand, antibody, chemokines, chimeric protein, endogenous adjuvant released from a dying tumor, and checkpoint inhibition proteins. In some embodiments, the adjuvant or adjuvant-like polypeptide is selected from the group comprising BCSP31, MOMP, FomA, MymA, ESAT6, PorB, PVL, Porin, OmpA, PepO, OmpU, Lumazine synthase, Omp16, Omp19, CobT, RpfE, Rv0652, HBHA, NhhA, DnaJ, Pneumolysin, Falgellin, IFN-alpha, IFN-gamma, IL-2, IL-12, IL-15, IL-18, IL-21, GM-CSF, IL-1b, IL-6, TNF-α, IL-7, IL-17, IL-1Beta, anti-CTLA4, anti-PD1, anti-41BB, PD-L1, Tim-3, Lag-3, TIGIT, GITR, and andti-CD3. In some embodiments, the adjuvant or adjuvant-like polypeptide is selected from Table 10.

In one aspect, provided herein is an RNA polynucleotide comprising, in the following order, a 3′ intron fragment and a triphosphorylated 5′ terminus. In some embodiments, the RNA polynucleotide comprises a 5′ spacer located upstream to the 3′ intron fragment and downstream from the triphosphorylated 5′ terminus.

In one aspect, provided herein is an RNA polynucleotide comprising a 5′ intron fragment and a triphosphorylated 5′ terminus. In some embodiments, the RNA polynucleotide comprises a 5′ spacer located downstream to the 5′ intron fragment.

In some embodiments, the RNA polynucleotide further comprises a monophosporylated 5′ terminus.

In one aspect, provided herein is an RNA polynucleotide comprising, in the following order, a 3′ intron fragment and a monophosphorylated 5′ terminus. In some embodiments, the RNA polynucleotide comprises a 5′ spacer located upstream to the 3′ intron fragment and downstream from the monophosphorylated 5′ terminus.

In one aspect, provided herein is an RNA polynucleotide comprising a 5′ intron fragment and a monophosphorylated 5′ terminus. In some embodiments, the RNA polynucleotide comprises a 5′ spacer located downstream to the 5′ intron fragment.

In some embodiments, the RNA polynucleotide further comprises a triphosphorylated 5′ terminus.

In some embodiments, the RNA polynucleotide further comprises a polyA purification tag. In some embodiments, the RNA polynucleotide further comprises an initiation sequence.

In one aspect, provided herein is an RNA preparation comprising: a. the circular RNA polynucleotide of claim 1, claim 2, or both; and b. a linear RNA polynucleotide comprising, at least one of the following. i. a 3′ intron polynucleotide comprising a monophosphorylated 5′ terminus and a 3′ intron fragment; ii. a 5′ intron polynucleotide comprising a monophosphorylated 5′ terminus and a 5′ intron fragment; iii. a 3′ intron polynucleotide comprising a triphosphorylated 5′ terminus and a 3′ intron fragment; and iv. a 5′ intron polynucleotide comprising a triphosphorylated 5′ terminus and a 3′ intron fragment, wherein the circular RNA polynucleotide comprises at least 90% of the RNA preparation.

In some embodiments, the 3′ intron polynucleotide or 5′ intron polynucleotide comprises a spacer. In some embodiments, the 3′ intron polynucleotide or 5′ intron polynucleotide comprises a polyA sequence. In some embodiments, the 3′ intron polynucleotide or 5′ intron polynucleotide comprises a UTR. In some embodiments, wherein the 3′ intron polynucleotide or 5′ intron polynucleotide comprises an IRES.

In one aspect, provided herein is a pharmaceutical composition comprising a circular RNA polynucleotide disclosed herein, a diluent, and optionally a salt buffer.

In one aspect, provided herein is a pharmaceutical composition comprising an RNA preparation disclosed herein, a diluent, and optionally a salt buffer.

In one aspect, provided herein is a pharmaceutical composition comprising a circular RNA polynucleotide disclosed herein, and a polycationic, cationic, or polymeric compound.

In one aspect, provided herein is a pharmaceutical composition comprising an RNA preparation of disclosed herein, and a polycationic, cationic, or polymeric compound.

In some embodiments, the polycationic or cationic compound is selected from the group consisting of: cationic peptides or proteins, basic polypeptides, cell penetrating peptides (CPPs), Tat-derived peptides, Penetratin, VP22 derived or analog peptides, Pestivirus Ems, HSV, VP22 (Herpes simplex), MAP, KALA or protein transduction domains (PTDs), PpT620, proline-rich peptides, arginine-rich peptides, lysine-rich peptides, MPG-peptide(s), Pep-1, L-oligomers, Calcitonin peptide(s), Antennapedia-derived peptides, pAntp, plsl, FGF, Lactoferrin, Transportan, Buforin-2, Bac715-24, SynB, SynB(1), pVEC, hCT-derived peptides, SAP, histones, cationic polysaccharides, cationic polymers, cationic lipids, dendrimers, polyimine, polyallylamine, oligofectamine, or cationic or polycationic polymers, sugar backbone based polymers, silan backbone based polymers, modified polyaminoacids, modified acrylates, modified polybetaminoester (PBAE), modified amidoamines, dendrimers, blockpolymers consisting of a combination of one or more cationic blocks and of one or more hydrophilic or hydrophobic blocks. In some embodiments, the polymeric compound is selected from the group consisting of: polyamines, polyethers, polyamides, polyesters, polycarbamates, polyureas, polycarbonates, polystyrenes, polyimides, polysulfones, polyurethanes, polyacetylenes, polyethylenes, polyethyleneimines, polyisocyanates, polyacrylates, polymethacrylates, polyacrylonitriles, and polyarylates. For example, a polymer may include poly(caprolactone) (PCL), ethylene vinyl acetate polymer (EVA), poly(lactic acid) (PLA), poly(L-lactic acid) (PLLA), poly(glycolic acid) (PGA), poly(lactic acid-co-glycolic acid) (PLGA), poly(L-lactic acid-co-glycolic acid) (PLLGA), poly(D,L-lactide) (PDLA), poly(Llactide) (PLLA), poly(D,L-lactide-co-caprolactone), poly(D,L-lactide-co-caprolactone-coglycolide), poly(D,L-lactide-co-PEO-co-D,L-lactide), poly(D,L-lactide-co-PPO-co-D,L-lactide), polyalkyl cyanoacrylate, polyurethane, poly-L-lysine (PLL), hydroxypropyl methacrylate (HPMA), polyethyleneglycol, poly-L-glutamic acid, poly(hydroxy acids), polyanhydrides, polyorthoesters, poly(ester amides), polyamides, poly(ester ethers), polycarbonates, polyalkylenes such as polyethylene and polypropylene, polyalkylene glycols such as poly(ethylene glycol) (PEG), polyalkylene oxides (PEO), polyalkylene terephthalates such as poly(ethylene terephthalate), polyvinyl alcohols (PVA), polyvinyl ethers, polyvinyl esters such as poly(vinyl acetate), polyvinyl halides such as poly(vinyl chloride) (PVC), polyvinylpyrrolidone (PVP), polysiloxanes, polystyrene, polyurethanes, derivatized celluloses such as alkyl celluloses, hydroxyalkyl celluloses, cellulose ethers, cellulose esters, nitro celluloses, hydroxypropylcellulose, carboxymethylcellulose, polymers of acrylic acids, such as poly(methyl(meth)acrylate) (PMMA), poly(ethyl(meth)acrylate), poly(butyl(meth)acrylate), 363 5 10 15 20 25 30 35 WO 2021/076805 PCT/US2020/055844 poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), poly(octadecyl acrylate) and copolymers and mixtures thereof, polydioxanone and its copolymers, polyhydroxyalkanoates, polypropylene flimarate, polyoxymethylene, poloxamers, poloxamines, poly(ortho)esters, poly(butyric acid), poly(valeric acid), poly(lactide-co-caprolactone), trimethylene carbonate, poly(N-acryloylmorpholine) (PAcM), poly(2-methyl-2-oxazoline) (PMOX), poly(2-ethyl-2-oxazoline) (PEOZ), and polyglycerol. In some embodiments, the polycationic or cationic compound is selected from the group comprising: protamine, nucleoline, spermine or spermidine, poly-L-lysine (PLL), polyarginine, HIV-binding peptides, HIV-1 Tat (HIV), polyethyleneimine (PEI), DOTMA: [1-(2,3-sioleyloxy)propyl)]-N,N,N-trimethylammonium chloride, DMRIE, di-C14-amidine, DOTIM, SAINT, DC-Chol, BGTC, CTAP, DOPC, DODAP, DOPE: Dioleyl phosphatidylethanol-amine, DOSPA, DODAB, DOIC, DMEPC, DOGS: Dioctadecylamidoglicylspermin, DIMRI: Dimyristooxypropyl dimethyl hydroxyethyl ammonium bromide, DOTAP: dioleoyloxy-3-(trimethylammonio)propane, DC-6-14: O,O-ditetradecanoyl-N-.alpha.-trimethylammonioacetyl)diethanolamine chloride, CLIP 1: rac-[(2,3-dioctadecyloxypropyl)(2-hydroxyethyl)]-dimethylammonium chloride, CLIP6: rac-[2(2,3-dihexadecyloxypropyloxymethyloxy)ethyl]-trimethylammonium, CLIP9: rac-[2(2,3-dihexadecyloxypropyloxysuccinyloxy)ethyl]-trimethylammonium, beta-aminoacid-polymers or reversed polyamides, PVP (poly(N-ethyl-4-vinylpyridinium bromide)), pDMAEMA (poly(dimethylaminoethyl methylacrylate)), pAMAM (poly(amidoamine)), diamine end modified 1,4 butanediol diacrylate-co-5-amino-1-pentanol polymers, polypropylamine dendrimers or pAMAM based dendrimers, polyimine(s), PEI: poly(ethyleneimine), poly(propyleneimine), polyallylamine, cyclodextrin based polymers, dextran based polymers, chitosan, and PMOXA-PDMS copolymers.

In an aspect, provided herein is a pharmaceutical composition comprising a circular RNA polynucleotide disclosed herein, a nanoparticle, and optionally a targeting moiety operably connected to the nanoparticle.

In an aspect, provided herein is a pharmaceutical composition comprising a RNA preparation disclosed herein, a nanoparticle, and optionally a targeting moiety operably connected to the nanoparticle.

In some embodiments, the nanoparticle is a lipid nanoparticle, a core-shell nanoparticle, a biodegradable nanoparticle, a biodegradable lipid nanoparticle, a polymer nanoparticle, or a biodegradable polymer nanoparticle. In certain embodiments, the nanoparticle comprises one or more cationic lipids selected from the group C12-200, MC3, DLinDMA, DLinkC2DMA, cKK-E12, ICE (Imidazol-based), HGT5000, HGT5001, DODAC, DDAB, DMRIE, DOSPA, DOGS, DODAP, DODMA and DMDMA, DODAC, DLenDMA, DMRIE, CLinDMA, CpLinDMA, DMOBA, DOcarbDAP, DLinDAP, DLincarbDAP, DLinCDAP, KLin-K-DMA, DLin-K-XTC2-DMA, HGT4003, and combinations thereof.

In some embodiments, the pharmaceutical composition comprises a targeting moiety, wherein the targeting moiety mediates receptor-mediated endocytosis or direct fusion into selected cells of a selected cell population or tissue in the absence of cell isolation or purification. In some embodiments, the targeting moiety is a scFv, nanobody, peptide, minibody, polynucleotide aptamer, heavy chain variable region, light chain variable region or fragment thereof. In some embodiments, the circular RNA polynucleotide or RNA preparation is in an amount effective to treat an infection (e.g., a viral infection) in a human subject in need thereof. In some embodiments, the pharmaceutical composition has an enhanced safety profile when compared to a pharmaceutical composition comprising vectors comprising exogenous DNA encoding antigens. In some embodiments, less than 1%, by weight, of the polynucleotides in the composition are double stranded RNA, DNA splints, or triphosphorylated RNA. In some embodiments, less than 1%, by weight, of the polynucleotides and proteins in the pharmaceutical composition are double stranded RNA, DNA splints, triphosphorylated RNA, phosphatase proteins, protein ligases, and capping enzymes.

In an aspect, provided herein is a method of treating a subject in need thereof comprising administering a therapeutically effective amount of a composition comprising the circular RNA polynucleotide disclosed herein, a nanoparticle, and optionally a targeting moiety operably connected to the nanoparticle. In an aspect, provided herein is a method of treating a subject in need thereof comprising administering a therapeutically effective amount of a composition comprising the RNA preparation disclosed herein, a nanoparticle, and optionally a targeting moiety operably connected to the nanoparticle.

In some embodiments, the subject has an infection (e.g., a viral infection). In certain embodiments, the method of treating a subject in need further comprises co-administration of an anti-inflammatory agent.

In some embodiments, composition comprises a targeting moiety, wherein the targeting moiety mediates receptor-mediated endocytosis into selected cells of a selected cell population in the absence of cell isolation or purification. In some embodiments, the targeting moiety is an scFv, nanobody, peptide, minibody, heavy chain variable region, light chain variable region or fragment thereof. In some embodiments, the composition comprises a targeting moiety, wherein the targeting moiety mediates receptor-mediated endocytosis into selected cells of a selected cell population in the absence of cell isolation or purification.

In some embodiments, the nanoparticle is a lipid nanoparticle, a core-shell nanoparticle, or a biodegradable nanoparticle. In some embodiments, the nanoparticle comprises one or more cationic lipids, ionizable lipids, or poly β-amino esters. In some embodiments, the nanoparticle comprises one or more non-cationic lipids. In some embodiments, the nanoparticle comprises one or more PEG-modified lipids, polyglutamic acid lipids, or hyaluronic acid lipids. In some embodiments, the nanoparticle comprises cholesterol. In some embodiments, the nanoparticle comprises arachidonic acid or oleic acid. In some embodiments, the nanoparticle encapsulates more than one circular RNA polynucleotide.

In an aspect, provided herein is a vector for making a circular RNA polynucleotide, comprising, in the following order, a 5′ duplex forming region, a 3′ Group I intron fragment, an Internal Ribosome Entry Site (IRES), an expression sequence encoding for one or more adjuvants, antigens, or adjuvant-like or antigen-like polypeptides, or fragments thereof, a 5′ Group I intron fragment, and a 3′ duplex forming region.

In an aspect, provided herein is a vector for making a circular RNA polynucleotide comprising, in the following order, a 5′ duplex forming region, a 3′ Group I intron fragment, an Internal Ribosome Entry Site (IRES), a noncoding sequence, a 5′ Group I intron fragment, and a 3′ duplex forming region.

In some embodiments, the vector comprises a first spacer between the 5′ duplex forming region and the 3′ group I intron fragment, and a second spacer between the 5′ group I intron fragment and the 3′ duplex forming region. In some embodiments, the first and second spacers each have a length of about 20 to about 60 nucleotides. In certain embodiments, the first and second spacers each comprise an unstructured region at least 5 nucleotides long. In some embodiments, the first and second spacers each comprise a structured region at least 7 nucleotides long. In some embodiments, the first and second duplex forming regions each have a length of about 9 to 50 nucleotides. In some embodiments, the vector is codon optimized. In certain embodiments, the vector is lacking at least one microRNA binding site present in an equivalent pre-optimization polynucleotide.

In an aspect, provided herein is a prokaryotic cell comprising a vector disclosed herein. In an aspect, provided herein is a eukaryotic cell comprising a circular RNA polynucleotide disclosed herein. In some embodiments, the eukaryotic cell is a human cell. In some embodiments, the eukaryotic cell is an antigen presentic cell.

In an aspect, provided herein is a vaccine, comprising: at least one circular RNA polynucleotide having an expression sequence encoding at least one viral antigenic polypeptide, adjuvant or adjuvant-like polypeptide, or an immunogenic fragment thereof, formulated in a lipid nanoparticle. In some embodiments, the adjuvant or adjuvant-like polypeptide is selected from Table 10. In some embodiments, the antigenic polypeptide is a viral polypeptide from an adenovirus; Herpes simplex, type 1; Herpes simplex, type 2; encephalitis virus, papillomavirus, Varicella-zoster virus; Epstein-barr virus; Human cytomegalovirus; Human herpes virus, type 8; Human papillomavirus; BK virus; JC virus; Smallpox; polio virus; Hepatitis B virus; Human bocavirus; Parvovirus B19; Human astrovirus; Norwalk virus; coxsackievirus; hepatitis A virus; poliovirus; rhinovirus; Severe acute respiratory syndrome virus; Hepatitis C virus; Yellow Fever virus; Dengue virus; West Nile virus; Rubella virus; Hepatitis E virus; Human Immunodeficiency virus (HIV); Influenza virus; Guanarito virus; Junin virus; Lassa virus; Machupo virus; Sabia virus; Crimean-Congo hemorrhagic fever virus; Ebola virus; Marburg virus; Measles virus; Mumps virus; Parainfluenza virus; Respiratory syncytial virus; Human metapneumo virus; Hendra virus; Nipah virus, Rabies virus; Hepatitis D; Rotavirus; Orbivirus; Coltivirus; Banna virus; Human Enterovirus; Hanta virus; West Nile virus, Middle East Respiratory Syndrome Corona Virus; Japanese encephalitis virus; Vesicular exanthernavirus; SARS-CoV-2; Eastern equine encephalitis, or a combination of any two or more of the foregoing. In some embodiments, the viral antigenic polypeptide or an immunogenic fragment thereof is selected or derived from any one of SEQ ID NOs: 325-336. In some embodiments, the viral antigenic polypeptide or an immunogenic fragment thereof has an amino acid sequence that has at least 90% identity to an amino acid sequence of any one of SEQ ID NOs: 325-336, and wherein the antigenic polypeptide or immunogenic fragment thereof has membrane fusion activity, attaches to cell receptors, causes fusion of viral and mammalian cellular membranes, and/or is responsible for binding of the virus to a cell being infected.

In an aspect, provided herein is a SARS-CoV2 vaccine, comprising: at least one circular RNA polynucleotide having an expression sequence encoding at least one SARS-CoV2 viral antigenic polypeptide or an immunogenic fragment thereof, formulated in a lipid nanoparticle. In some embodiments, the SARS-CoV2 viral antigenic polypeptide is selected from: SARS-CoV2 spike protein, Nsp1-Nsp16, ORF3a, ORF6, ORF7a, ORFb, ORF8, ORF10, SARS-CoV2 envelope protein, SARS-CoV2 Membrane protein, SARS-CoV2 nucleocapsid protein or any antigenic peptide of SARS-CoV2 or fragment of SARS-CoV2 peptide. In some embodiments, the SARS-CoV2 viral antigenic polypeptide is derived from SARS-CoV2 virus strain G, strain GR, strain GH, strain L, strain V, or a combination thereof.

In some embodiments, the expression sequence comprised in a vaccine disclosed herein (e.g., a SARS-CoV2 vaccine) is codon-optimized. In some embodiments, the vaccine (e.g., SARS-CoV2 vaccine) is multivalent. In some embodiments, the vaccine (e.g., SARS-CoV2 vaccine) is formulated in an effective amount to produce an antigen-specific immune response.

In some embodiments, the circular RNA polynucleotide comprises a first expression sequence encoding a first viral antigenic polypeptide and a second expression sequence encoding a second viral antigenic polypeptide.

In one aspect, provided herein is a method of inducing an immune response in a subject, the method comprising administering to the subject a vaccine disclosed herein, in an amount effective to produce an antigen-specific immune response in the subject. In an aspect, provided herein is a method of inducing an immune response in a subject, the method comprising administering to the subject a SARS-CoV2 disclosed herein, in an amount effective to produce an antigen-specific immune response in the subject.

In some embodiments, the antigen-specific immune response comprises a T cell response or a B cell response. In some embodiments, the subject is administered a single dose of the vaccine. In some embodiments, the subject is administered a booster dose of the vaccine. In some embodiments, the vaccine is administered to the subject by intranasal administration, intradermal injection or intramuscular injection. In some embodiments, an anti-antigenic polypeptide antibody titer produced in the subject is increased by at least 1 log relative to a pre-determined threshold level. In some embodiments, an anti-antigenic polypeptide antibody titer produced in the subject is increased by 1-3 log relative to a pre-determined threshold level. In some embodiments, the anti-antigenic polypeptide antibody titer produced in the subject is increased at least 2 times relative to a pre-determined threshold level. In some embodiments, the anti-antigenic polypeptide antibody titer produced in the subject is increased 2-10 times relative to a pre-determined threshold level. In some embodiments, the pre-determined threshold level is an anti-antigenic polypeptide antibody titer produced in a subject who has not been administered a vaccine comprising the antigenic polypeptide. In some embodiments, the pre-determined threshold level is an anti-antigenic polypeptide antibody titer produced in a subject who has been administered a live attenuated vaccine or an inactivated vaccine comprising the antigenic polypeptide. In some embodiments, the pre-determined threshold level is an anti-antigenic polypeptide antibody titer produced in a subject who has been administered a recombinant protein vaccine or purified protein vaccine comprising the antigenic polypeptide.

In one aspect, provided herein is a circular RNA polynucleotide having an expression sequence encoding at least one viral antigenic polypeptide, adjuvant or adjuvant-like polypeptide, or an immunogenic fragment thereof. In one aspect, provided herein is an expression vector comprising an engineered nucleic acid encoding at least one circular RNA polynucleotide disclosed herein.

In one aspect, provided herein is a circular RNA polynucleotide vaccine comprising the circular RNA polynucleotide disclosed herein, formulated in a lipid nanoparticle. In some embodiments, the nanoparticle has a mean diameter of 50-200 nm. In some embodiments, the lipid nanoparticle comprises a cationic lipid, a PEG-modified lipid, a sterol and a non-cationic lipid. In some embodiments, the lipid nanoparticle carrier comprises a molar ratio of about 20-60% cationic lipid, 0.5-15% PEG-modified lipid, 25-55% sterol, and 25% non-cationic lipid. In some embodiments, the cationic lipid is an ionizable cationic lipid and the non-cationic lipid is a neutral lipid, and the sterol is a cholesterol. In some embodiments, the cationic lipid is selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319). In some embodiments, the nanoparticle has a polydispersity value of less than 0.4. In some embodiments, the nanoparticle has a net neutral charge at a neutral pH value.

In some embodiments of a disclosed vaccine, the circular RNA polynucleotide is co-formulated with an adjuvant in the same nanoparticle. In some embodiments, the adjuvant is CpG, imiquimod, Aluminium, or Freund's adjuvant.

In one aspect, provided herein is a pharmaceutical composition for use in vaccination of a subject, comprising an effective dose of circular RNA polynucleotide encoding at least one viral antigen or adjuvant or adjuvant-like polypeptide, or an immunogenic fragment thereof, wherein the effective dose is sufficient to produce a 1,000-10,000 neutralization titer produced by neutralizing antibody against said antigen or adjuvant or adjuvant-like polypeptide, or an immunogenic fragment thereof, as measured in serum of the subject at 1-72 hours post administration. In one aspect, provided herein is a pharmaceutical composition for use in vaccination of a subject, comprising an effective dose of circular mRNA encoding at least one viral antigen or adjuvant or adjuvant-like polypeptide, or an immunogenic fragment thereof, wherein the effective dose is sufficient to produce detectable levels of antigen or adjuvant or adjuvant-like polypeptide, or an immunogenic fragment thereof, as measured in serum of the subject at 1-72 hours post administration. In some embodiments, the pharmaceutical composition is for use in a method of inducing an antigen specific immune response in a subject, the method comprising administering to the subject the vaccine or the pharmaceutical composition in an amount effective to produce an antigen specific immune response in the subject.

In one aspect, provided herein is a method of inducing, producing, or enhancing an immune response in a subject, the method comprising administering to the subject the pharmaceutical composition disclosed herein, in an amount effective to induce, produce or enhance an antigen-specific immune response in the subject. In some embodiments, the pharmaceutical composition immunizes the subject against the virus for up to 2 years. In some embodiments, the pharmaceutical composition immunizes the subject against the virus for more than 2 years. In some embodiments, the subject has been exposed to the virus, wherein the subject is infected with the virus, or wherein the subject is at risk of infection by the virus. In some embodiments, the subject is immunocompromised.

In one aspect, provided herein is the use of a vaccine or pharmaceutical composition disclosed herein, in the manufacture of a medicament for use in a method of inducing an antigen specific immune response in a subject, the method comprising administering to the subject the vaccine in an amount effective to produce an antigen specific immune response in the subject.

In one aspect, provided herein is a method of inducing cross-reactivity against a variety of viruses or strains of a virus in a mammal, the method comprising administering to the mammal in need thereof the vaccine of any preceding claim or the pharmaceutical composition of any preceding claim. In some embodiments, the method comprises administering at least two circular RNA polynucleotides having an expression sequence each encoding a consensus viral antigen to the mammal separately. In some embodiments, the method comprises administering at least two circular RNA polynucleotides having an expression sequence each encoding a consensus viral antigen to the mammal simultaneously. In some embodiments, the method comprises.

### BRIEF DESCRIPTION OF THE DRAWINGS

FIG. 1 depicts luminescence in supernatants of HEK293 (FIGS. 1A, 1D, and 1E), HepG2 (FIG. 1B), or 1C1C7 (FIG. 1C) cells 24 hours after transfection with circular RNA comprising a Gaussia luciferase expression sequence and various IRES sequences.

FIG. 2 depicts luminescence in supernatants of HEK293 (FIG. 2A), HepG2 (FIG. 2B), or 1C1C7 (FIG. 2C) cells 24 hours after transfection with circular RNA comprising a Gaussia luciferase expression sequence and various IRES sequences having different lengths.

FIG. 3 depicts stability of select IRES constructs in HepG2 (FIG. 3A) or IC1C7 (FIG. 3B) cells over 3 days as measured by luminescence.

FIGS. 4A and 4B depict protein expression from select IRES constructs in Jurkat cells, as measured by luminescence from secreted Gaussia luciferase in cell supernatants.

FIGS. 5A and 5B depict stability of select IRES constructs in Jurkat cells over 3 days as measured by luminescence.

FIG. 6 depicts comparisons of 24 hour luminescence (FIG. 6A) or relative luminescence over 3 days (FIG. 6B) of modified linear, unpurified circular, or purified circular RNA encoding Gaussia luciferase.

FIG. 7 depicts transcript induction of IFNγ (FIG. 7A), IL-6 (FIG. 7B), IL-2 (FIG. 7C), RIG-I (FIG. 7D), IFN-β1 (FIG. 7E), and TNFα (FIG. 7F) after electroporation of Jurkat cells with modified linear, unpurified circular, or purified circular RNA.

FIG. 8 depicts a comparison of luminescence of circular RNA and modified linear RNA encoding Gaussia luciferase in human primary monocytes (FIG. 8A) and macrophages (FIG. 8B and FIG. 8C).

FIG. 9 depicts relative luminescence over 3 days (FIG. 9A) in supernatant of primary T cells after transduction with circular RNA comprising a Gaussia luciferase expression sequence and varying IRES sequences or 24 hour luminescence (FIG. 9B).

FIG. 10 depicts 24 hour luminescence in supernatant of primary T cells (FIG. 10A) after transduction with circular RNA or modified linear RNA comprising a gaussia luciferase expression sequence, or relative luminescence over 3 days (FIG. 10B), and 24 hour luminescence in PBMCs (FIG. 10C).

FIG. 11 depicts HPLC chromatograms (FIG. 11A) and circularization efficiencies (FIG. 11B) of RNA constructs having different permutation sites.

FIG. 12 depicts HPLC chromatograms (FIG. 12A) and circularization efficiencies (FIG. 12B) of RNA constructs having different introns and/or permutation sites.

FIG. 13 depicts HPLC chromatograms (FIG. 13A) and circularization efficiencies (FIG. 13B) of 3 RNA constructs with or without homology arms.

FIG. 14 depicts circularization efficiencies of 3 RNA constructs without homology arms or with homology arms having various lengths and GC content.

FIGS. 15A and 15B depict HPLC HPLC chromatograms showing the contribution of strong homology arms to improved splicing efficiency, the relationship between circularization efficiency and nicking in select constructs, and combinations of permutations sites and homology arms hypothesized to demonstrate improved circularization efficiency.

FIG. 16 shows fluorescent images of T cells mock electroporated (left) or electroporated with circular RNA encoding a CAR (right) and co-cultured with Raji cells expressing GFP and firefly luciferase.

FIG. 17 shows bright field (left), fluorescent (center), and overlay (right) images of T cells mock electroporated (top) or electroporated with circular RNA encoding a CAR (bottom) and co-cultured with Raji cells expressing GFP and firefly luciferase.

FIG. 18 depicts specific lysis of Raji target cells by T cells mock electroporated or electroporated with circular RNA encoding different CAR sequences.

FIG. 19 depicts luminescence in supernatants of Jurkat cells (left) or resting primary human CD3+ T cells (right) 24 hours after transduction with linear or circular RNA comprising a Gaussia luciferase expression sequence and varying IRES sequences (FIG. 19A), and relative luminescence over 3 days (FIG. 19B).

FIG. 20 depicts transcript induction of IFN-β1 (FIG. 20A), RIG-I (FIG. 20B), IL-2 (FIG. 20C), IL-6 (FIG. 20D), IFNγ (FIG. 20E), and TNFα (FIG. 20F) after electroporation of human CD3+ T cells with modified linear, unpurified circular, or purified circular RNA.

FIG. 21 depicts specific lysis of Raji target cells by human primary CD3+ T cells electroporated with circRNA encoding a CAR as determined by detection of firefly luminescence (FIG. 21A), and IFNγ transcript induction 24 hours after electroporation with different quantities of circular or linear RNA encoding a CAR sequence (FIG. 21B).

FIG. 22 depicts specific lysis of target or non-target cells by human primary CD3+ T cells electroporated with circular or linear RNA encoding a CAR at different E:T ratios (FIG. 22A and FIG. 22B) as determined by detection of firefly luminescence.

FIG. 23 depicts specific lysis of target cells by human CD3+ T cells electroporated with RNA encoding a CAR at 1, 3, 5, and 7 days post electroporation.

FIG. 24 depicts specific lysis of target cells by human CD3+ T cells electroporated with circular RNA encoding a CD19 or BCMA targeted CAR.

FIG. 25 depicts total Flux of organs harvested from CD-1 mice dosed with circular RNA encoding FLuc and formulated with 50% Lipid 10b-15, 10% DSPC, 1.5% PEG-DMG, and 38.5% cholesterol.

FIG. 26 shows images highlighting the luminescence of organs harvested from CD-1 mice dosed with circular RNA encoding FLuc and formulated with 50% Lipid 10b-15, 10% DSPC, 1.5% PEG-DMG, and 38.5% cholesterol.

FIG. 27 depicts molecular characterization of Lipids 10a-26 and 10a-27. FIG. 27A shows the proton nuclear magnetic resonance (NMR) spectrum of Lipid Lipid 10a-26. FIG. 27B shows the retention time of Lipid 10a-26 measured by liquid chromatography-mass spectrometry (LC-MS). FIG. 27C shows the mass spectrum of Lipid 10a-26. FIG. 27D shows the proton NMR spectrum of Lipid 10a-27. FIG. 27E shows the retention time of Lipid 10a-27 measured by LC-MS. FIG. 27F shows the mass spectrum of Lipid 10a-27.

FIG. 28 depicts molecular characterization of Lipid 22-S14 and its synthetic intermediates. FIG. 28A depicts the NMR spectrum of 2-(tetradecylthio)ethan-1-ol. FIG. 28B depicts the NMR spectrum of 2-(tetradecylthio)ethyl acrylate. FIG. 28C depicts the NMR spectrum of bis(2-(tetradecylthio)ethyl) 3,3′-((3-(2-methyl-1H-imidazol-1-yl)propyl)azanediyl)dipropionate (Lipid 22-S14).

FIG. 29 depicts the NMR spectrum of bis(2-(tetradecylthio)ethyl) 3,3′-((3-(1H-imidazol-1-yl)propyl)azanediyl)dipropionate (Lipid 93-S14).

FIG. 30 depicts molecular characterization of heptadecan-9-yl 8-((3-(2-methyl-1H-imidazol-1-yl)propyl)(8-(nonyloxy)-8-oxooctyl)amino)octanoate (Lipid 10a-54). FIG. 30A shows the proton NMR spectrum of Lipid 10a-54. FIG. 30B shows the retention time of Lipid 10a-54measured by LC-MS. FIG. 30C shows the mass spectrum of Lipid 10a-54.

FIG. 31 depicts molecular characterization of heptadecan-9-yl 8-((3-(1H-imidazol-1-yl)propyl)(8-(nonyloxy)-8-oxooctyl)amino)octanoate (Lipid 10a-53). FIG. 31A shows the proton NMR spectrum of Lipid 10a-53. FIG. 31B shows the retention time of Lipid 10a-53 measured by LC-MS. FIG. 31C shows the mass spectrum of Lipid 10a-53.

FIG. 32A depicts total flux of spleen and liver harvested from CD-1 mice dosed with circular RNA encoding firefly luciferase (FLuc) and formulated with ionizable lipid of interest, DSPC, cholesterol, and DSPE-PEG 2000 (Avanti Polar Lipids Inc.) at a weight ratio of 16:1:4:1 or 62:4:33:1 molar ratio. FIG. 32B depicts average radiance for biodistribution of protein expression.

FIG. 33A depicts images highlighting the luminescence of organs harvested from CD-1 mice dosed with circular RNA encoding FLuc and formulated with ionizable Lipid 22-S14, DSPC, cholesterol, and DSPE-PEG 2000 (Avanti Polar Lipids Inc.) at a weight ratio of 16:1:4:1 or 62:4:33:1 molar ratio. FIG. 33B depicts whole body IVIS images of CD-1 mice dosed with circular RNA encoding FLuc and formulated with ionizable Lipid 22-S14, DSPC, cholesterol, and DSPE-PEG 2000 (Avanti Polar Lipids Inc.) at a weight ratio of 16:1:4:1 or 62:4:33:1 molar ratio.

FIG. 34A depicts images highlighting the luminescence of organs harvested from CD-1 mice dosed with circular RNA encoding FLuc and formulated with ionizable Lipid 93-S14, DSPC, cholesterol, and DSPE-PEG 2000 (Avanti Polar Lipids Inc.) at a weight ratio of 16:1:4:1 or 62:4:33:1 molar ratio. FIG. 34B depicts whole body IVIS images of CD-1 mice dosed with circular RNA encoding FLuc and formulated with ionizable Lipid 93-S14, DSPC, cholesterol, and DSPE-PEG 2000 (Avanti Polar Lipids Inc.) at a weight ratio of 16:1:4:1 or 62:4:33:1 molar ratio.

FIG. 35A depicts images highlighting the luminescence of organs harvested from CD-1 mice dosed with circular RNA encoding FLuc and formulated with ionizable Lipid 10a-26, DSPC, cholesterol, and DSPE-PEG 2000 (Avanti Polar Lipids Inc.) at a weight ratio of 16:1:4:1 or 62:4:33:1 molar ratio. FIG. 35B depicts whole body IVIS images of CD-1 mice dosed with circular RNA encoding FLuc and formulated with ionizable Lipid 10a-26, DSPC, cholesterol, and DSPE-PEG 2000 (Avanti Polar Lipids Inc.) at a weight ratio of 16:1:4:1 or 62:4:33:1 molar ratio.

FIG. 36 depicts images highlighting the luminescence of organs harvested from c57BL/6J mice dosed with circular RNA encoding FLuc and encapsulated in lipid nanoparticles formed with Lipid 10b-15 (FIG. 36A), Lipid 10a-53 (FIG. 36B), or Lipid 10a-54 (FIG. 36C). PBS was used as control (FIG. 36D).

FIGS. 37A and 37B depict relative luminescence in the lysates of human PBMCs after 24-hour incubation with testing lipid nanoparticles containing circular RNA encoding firefly luciferase.

FIG. 38 shows the expression of GFP (FIG. 37A) and CD19 CAR (FIG. 37B) in human PBMCs after incubating with testing lipid nanoparticle containing circular RNA encoding either GFP or CD19 CAR.

FIG. 39 depicts the expression of an anti-murine CD19 CAR in 1C1C7 cells lipotransfected with circular RNA comprising an anti-murine CD19 CAR expression sequence and varying IRES sequences.

FIG. 40 shows the cytotoxicity of an anti-murine CD19 CAR to murine T cells. The CD19 CAR is encoded by and expressed from a circular RNA, which is electroporated into the murine T cells.

FIG. 41 depicts the B cell counts in peripheral blood (FIGS. 40A and 40B) or spleen (FIG. 40C) in C57BL/6J mice injected every other day with testing lipid nanoparticles encapsulating a circular RNA encoding an anti-murine CD19 CAR.

FIGS. 42A and 42B compares the expression level of an anti-human CD19 CAR expressed from a circular RNA with that expressed from a linear mRNA.

FIGS. 43A and 43B compares the cytotoxic effect of an anti-human CD19 CAR expressed from a circular RNA with that expressed from a linear mRNA

FIG. 44 depicts the cytotoxicity of two CARs (anti-human CD19 CAR and anti-human BCMA CAR) expressed from a single circular RNA in T cells.

FIG. 45A shows representative FACS plots with frequencies of tdTomato expression in various spleen immune cell subsets following treatment with LNPs formed with Lipid 10a-27 or 10a-26 or Lipid 10b-15. FIG. 45B shows the quantification of the proportion of myeloid cells, B cells, and T cells expressing tdTomato (mean+std. dev., n=3), equivalent to the proportion of each cell population successfully transfected with Cre circular RNA. FIG. 45C illustrates the proportion of additional splenic immune cell populations, including NK cells, classical monocytes, nonclassical monocytes, neutrophils, and dendritic cells, expressing tdTomato after treatment with Lipids 27 and 26 (mean+std. dev., n=3).

FIG. 46A depicts an exemplary RNA construct design with built-in polyA sequences in the introns. FIG. 46B shows the chromatography trace of unpurified circular RNA. FIG. 46C shows the chromatography trace of affinity-purified circular RNA. FIG. 46D shows the immunogenicity of the circular RNAs prepared with varying IVT conditions and purification methods. (Commercial=commercial IVT mix; Custom=customerized IVT mix; Aff=affinity purification; Enz=enzyme purification; GMP:GTP ratio=8, 12.5, or 13.75).

FIG. 47A depicts an exemplary RNA construct design with a dedicated binding sequence as an alternative to polyA for hybridization purification. FIG. 47B shows the chromatography trace of unpurified circular RNA. FIG. 46C shows the chromatography trace of affinity-purified circular RNA.

FIG. 48A shows the chromatography trace of unpurified circular RNA encoding dystrophin. FIG. 48B shows the chromatography trace of enzyme-purified circular RNA encoding dystrophin.

FIG. 49 compares the expression (FIG. 49A) and stability (FIG. 49B) of purified circRNAs with different 5′ spacers between the 3′ intron fragment/5′ internal duplex region and the IRES in Jurkat cells. (AC=only A and C were used in the spacer sequence; UC=only U and C were used in the spacer sequence.)

FIG. 50 shows luminescence expression levels and stability of expression in primary T cells from circular RNAs containing the original or modified IRES elements indicated.

FIG. 51 shows luminescence expression levels and stability of expression in HepG2 cells from circular RNAs containing the original or modified IRES elements indicated.

FIG. 52 shows luminescence expression levels and stability of expression in 1C1C7 cells from circular RNAs containing the original or modified IRES elements indicated.

FIG. 53 shows luminescence expression levels and stability of expression in HepG2 cells from circular RNAs containing IRES elements with untranslated regions (UTRs) inserted or hybrid IRES elements. “Scr” means Scrambled, which was used as a control.

FIG. 54 shows luminescence expression levels and stability of expression in 1C1C7 cells from circular RNAs containing an IRES and variable stop codon cassettes operably linked to a gaussia luciferase coding sequence.

FIG. 55 shows luminescence expression levels and stability of expression in 1C1C7 cells from circular RNAs containing an IRES and variable untranslated regions (UTRs) inserted before the start codon of a gaussian luciferase coding sequence.

FIG. 56 shows expression levels of human erythropoietin (hEPO) in Huh7 cells from circular RNAs containing two miR-122 target sites downstream from the hEPO coding sequence.

FIG. 57 shows luminescence expression levels in SupT1 cells (from a human T cell tumor line) and MV4-11 cells (from a human macrophage line) from LNPs transfected with circular RNAs encoding for Firefly luciferase in vitro.

FIG. 58 shows a comparison of transfected primary human T cells LNPs containing circular RNAs dependency of ApoE based on the different helper lipid, PEG lipid, and ionizable lipid:phosphate ratio formulations.

FIG. 59 shows uptake of LNP containing circular RNAs encoding eGFP into activated primary human T cells with or without the aid of ApoE3.

FIG. 60 shows immune cell expression from a LNP containing circular RNA encoding for a Cre fluorescent protein in a Cre reporter mouse model.

FIG. 61 shows immune cell expression of mOX40L in wildtype mice following intravenous injection of LNPs that have been transfected with circular RNAs encoding mOX40L.

FIG. 62 shows single dose of mOX40L in LNPs transfected with circular RNAs capable of expressing mOX40L. FIGS. 62A and 62B provide percent of mOX40L expression in splenic T cells, CD4+ T cells, CD8+ T cells, B cells, NK cells, dendritic cells, and other myloid cells. FIG. 62C provides mouse weight change 24 hours after transfection.

FIG. 63 shows B cell depletion of LNPs transfected intravenously with circular RNAs in mice. FIG. 63A quantifies Be cell depetion through B220+ B cells of live, CD45+ immune cells and FIG. 63B compares B cell depletion of B220+ B cells of live, CD45+ immune cells in comparison to luciferase expressing circular RNAs. FIG. 63C provides B cell weight gain of the transfected cells.

FIG. 64 shows CAR expression levels in the peripheral blood (FIG. 64A) and spleen (FIG. 64B) when treated with LNP encapsulating circular RNA that expresses anti-CD19 CAR. Anti-CD20 (aCD20) and circular RNA encoding luciferase (oLuc) were used for comparison.

FIG. 65 shows the overall frequency of anti-CD19 CAR expression, the frequency of anti-CD19 CAR expression on the surface of cells and effect on anti-tumor response of IRES specific circular RNA encoding anti-CD19 CARs on T-cells. FIG. 65A shows anti-CD19 CAR geometric mean florescence intensity, FIG. 65B shows percentage of anti-CD19 CAR expression, and FIG. 65C shows the percentage target cell lysis performed by the anti-CD19 CAR. (CK=Caprine Kobuvirus; AP=Apodemus Picornavirus; CK*=Caprine Kobuvirus with codon optimization; PV=Parabovirus; SV=Salivirus.)

FIG. 66 shows CAR expression levels of A20 FLuc target cells when treated with IRES specific circular RNA constructs.

FIG. 67 shows luminescence expression levels for cytosolic (FIG. 67A) and surface (FIG. 67B) proteins from circular RNA in primary human T-cells.

FIG. 68 shows luminescence expression in human T-cells when treated with IRES specific circular constructs. Expression in circular RNA constructs were compared to linear mRNA. FIG. 68A, FIG. 68B, and FIG. 68G provide Gaussia luciferase expression in multiple donor cells. FIG. 68C, FIG. 68D, FIG. 68E, and FIG. 68F provides firefly luciferase expression in multiple donor cells.

FIG. 69 shows anti-CD19 CAR (FIG. 69A and FIG. 69B) and anti-BCMA CAR (FIG. 68B) expression in human T-cells following treatment of a lipid nanoparticle encompassing a circular RNA that encodes either an anti-CD19 or anti-BCMA CAR to a firefly luciferase expressing K562 cell.

FIG. 70 shows anti-CD19 CAR expression levels resulting from delivery via electroporation in vitro of a circular RNA encoding an anti-CD19 CAR in a specific antigen-dependent manner. FIG. 70A shows Nalm6 cell lysing with an anti-CD19 CAR. FIG. 70B shows K562 cell lysing with an anti-CD19 CAR.

FIG. 71 shows transfection of LNP mediated by use of ApoE3 in solutions containing LNP and circular RNA expressing green fluorescence protein (GFP). FIG. 71A showed the live-dead results. FIG. 71B, FIG. 71C, FIG. 71D, and FIG. 71E provide the frequency of expression for multiple donors.

FIG. 72A, FIG. 72B, FIG. 72C, FIG. 72D, FIG. 72E, FIG. 72F, FIG. 72G, FIG. 72H, FIG. 72I, FIG. 72J, FIG. 72K, and FIG. 72L show total flux and precent expression for varying lipid formulations. See Example 74.

FIG. 73 shows circularization efficiency of an RNA molecule encoding a stabilized (double proline mutant) SARS-CoV2 spike protein. FIG. 73A shows the in vitro transcription product of the ˜4.5 kb SARS-CoV2 spike-encoding circRNA. FIG. 73B shows a histogram of spike protein surface expression via flow cytometry after transfection of spike-encoding circRNA into 293 cells. Transfected 293 cells were stained 24 hours after transfection with CR3022 primary antibody and APC-labeled secondary antibody. FIG. 73C shows a flow cytometry plot of spike protein surface expression on 293 cells after transfection of spike-encoding circRNA. Transfected 293 cells were stained 24 hours after transfection with CR3022 primary antibody and APC-labeled secondary antibody.

FIG. 74 provides multiple controlled adjuvant strategies. CircRNA as indicated on the figure entails an unpurified sense circular RNA splicing reaction using GTP as an indicator molecule in vitro. 3p-circRNA entails a purified sense circular RNA as well as a purified antisense circular RNA mixed containing triphosphorylated 5′ termini. FIG. 74A shows IFN-β Induction in vitro in wild type and MAVS knockout A549 cells and FIG. 74B shows in vivo cytokine response to formulated circRNA generated using the indicated strategy.

FIG. 75 illustrates an intramuscular delivery of LNP containing circular RNA constructs. FIG. 75A provides a live whole body flux post a 6 hour period and 75B provides whole body IVIS 6 hours following a 1 μg dose of the LNP-circular RNA construct. FIG. 75C provides an ex vivo expression distribution over a 24-hour period.

FIG. 76 illustrates expression of multiple circular RNAs from a single lipid formulation. FIG. 76A provides hEPO titers from a single and mixed set of LNP containing circular RNA constructs, while FIG. 76B provides total flux of bioluminescence expression from single or mixed set of LNP containing circular RNA constructs.

FIG. 77 illustrates SARS-CoV2 spike protein expression of circular RNA encoding spike SARS-CoV2 proteins. FIG. 77A shows frequency of spike CoV2 expression; FIG. 77B shows geometric mean fluorescence intensity (gMFI) of the spike CoV2 expression; and FIG. 77C compares gMFI expression of the construct to the frequency of expression.

### DETAILED DESCRIPTION

Described herein are compositions, methods, processes, kits and devices for the selection, design, preparation, manufacture, formulation, and/or use of circular RNA vaccines. The present invention additionally provides compositions, e.g., pharmaceutical compositions, comprising one or more circular RNA vaccines.

The circular RNA vaccines of the invention comprise one or more circular RNA polynucleotides, which encode one or more wild type or engineered proteins, peptides or polypeptides (e.g., adjuvant and antigens). In some embodiments, the infectious agent from which the adjuvant, adjuvant-like protein, and antigen is derived or engineered includes, but is not limited to viruses, bacteria, fungi, protozoa, and/or parasites.

In some embodiments are provided methods of inducing, eliciting, boosting or triggering an immune response in a cell, tissue or organism, comprising contacting said cell, tissue or organism with any of the circular RNA or linear mRNA vaccines described or taught herein.

Aspects of the invention provide circular RNA vaccines comprising one or more RNA polynucleotides having an expression sequence encoding a first antigenic polypeptide. In some embodiments, a circular RNA polynucleotides is formulated within a transfer vehicle (e.g., a lipid nanoparticle).

In some embodiments, the expression sequence is codon-optimized. In some embodiments, the first antigenic polypeptide is derived from an infectious agent. In some embodiments, the infectious agent is selected from a member of the group consisting of strains of viruses and strains of bacteria. In some embodiments, the one or more RNA polynucleotides encode a further antigenic polypeptide. In some embodiments, the further antigenic polypeptide is encoded by an RNA polynucleotide having a codon-optimized expression sequence.

In some embodiments, the one or more antigenic polypeptide is selected from those proteins listed in Table 9, or an antigenic fragment thereof. In some embodiments, the expression sequence of the one or more RNA polynucleotides and/or the expression sequence of the second RNA polynucleotide each, independently, encodes an antigenic polypeptide selected from Table 9, or an antigenic fragment thereof. In some embodiments, each expression sequence of the one or more RNA polynucleotides is selected from any of the RNA sequences listed in Table 9, or antigenic fragments thereof.

In some embodiments provided herein, the infectious agent is a strain of virus selected from the group consisting of adenovirus; Herpes simplex, type 1; Herpes simplex, type 2; encephalitis virus, papillomavirus, Varicella-zoster virus; Epstein-barr virus; Human cytomegalovirus; Human herpes virus, type 8; Human papillomavirus; BK virus; JC virus, Smallpox; polio virus; Hepatitis B virus; Human bocavirus; Parvovirus B19; Human astrovirus; Norwalk virus; coxsackievirus; hepatitis A virus; poliovirus; rhinovirus; Severe acute respiratory syndrome virus; Hepatitis C virus; Yellow Fever virus; Dengue virus; West Nile virus; Rubella virus; Hepatitis E virus; Human Immunodeficiency virus (HIV); Influenza virus; Guanarito virus; Junin virus; Lassa virus; Machupo virus; Sabia virus; Crimean-Congo hemorrhagic fever virus; Ebola virus; Marburg virus; Measles virus; Mumps virus; Parainfluenza virus; Respiratory syncytial virus; Human metapneumo virus; Hendra virus; Nipah virus; Rabies virus; Hepatitis D; Rotavirus; Orbivirus; Coltivirus; Banna virus; Human Enterovirus; Hanta virus; West Nile virus; Middle East Respiratory Syndrome Corona Virus; Japanese encephalitis virus; Vesicular exanthernavirus; and Eastern equine encephalitis.

In some embodiments, the virus is a strain of Influenza A or Influenza B or combinations thereof. In some embodiments, the strain of Influenza A or Influenza B is associated with birds, pigs, horses, dogs, humans or non-human primates. In some embodiments, the antigenic polypeptide encodes a hemagglutinin protein or fragment thereof. In some embodiments, the hemagglutinin protein is HI, H2, H3, H4, H5, H6, H7, H8, H9, H10, H11, H12, H13, H14, H15, H16, H17, H18, or a fragment thereof. In some embodiments, the hemagglutinin protein does not comprise a head domain (HA1). In some embodiments, the hemagglutinin protein comprises a portion of the head domain (HA 1). In some embodiments, the hemagglutinin protein does not comprise a cytoplasmic domain. In some embodiments, the hemagglutinin protein comprises a portion of the cytoplasmic domain. In some embodiments, the hemagglutinin protein is a truncated hemagglutinin protein. In some embodiments, the truncated hemagglutinin protein comprises a portion of the transmembrane domain. In some embodiments, the amino acid sequence of the hemagglutinin protein or fragment thereof comprises at least 90%, 91%, 92%, 93%, 94%, 95%, 96%, 97% 98%, or 99% sequence identity with any one of the hemagglutinin amino acid sequences provided in Table 9. In some embodiments, the virus is selected from the group consisting of H1N1, H3N2, H7N9, and H10N8.

In some embodiments, the infectious agent is a strain of bacteria selected from Mycobacterium tuberculosis, Clostridium difficile, Staphylococcus aureus, Enterococcus faecalis, Enterococcus faecium, Pseudomonas aeruginosa, and Acinetobacter baumannii. In some embodiments, the bacteria is resistant to one or more antibiotics. In some embodiments, the bacteria is Clostridium difficile. In some embodiments, the C. difficile is clindamycin resistant, and/or fluoroquinolone resistant. In some embodiments, the bacteria is S. aureus. In some embodiments, the S. aureus is methicillin resistant and/or vancomycin resistant.

In some embodiments, a circular RNA polynucleotide comprises more than one expression sequence. In some embodiments, an expression sequence may encode more than one antigenic polypeptide. In some embodiments, the expression sequence of the one or more RNA polynucleotides encode at least 2, 3, 4, 5, 6, 7, 8, 9 or 10 antigenic polypeptides. In some embodiments, the expression sequence of the one or more RNA polynucleotides encode at least 10, 15, 20 or 50 antigenic polypeptides. In some embodiments, the expression sequence of the one or more RNA polynucleotides encode 2-10, 10-15, 15-20, 20-50, 50-100 or 100-200 antigenic polypeptides.

In some embodiments, a circular RNA polynucleotide contains only naturally occurring nucleic acids.

Additional aspects provide a method of inducing an antigen specific immune response in a subject comprising administering any of the vaccines described herein to the subject in an effective amount to produce an antigen specific immune response. In some embodiments, the antigen specific immune response comprises a T cell response. In some embodiments, the antigen specific immune response comprises a B cell response. In some embodiments, the method of producing an antigen specific immune response involves a single administration of the vaccine. In some embodiments, the method further comprises administering one or more booster dose of the vaccine. In some embodiments, the vaccine is administered to the subject by intradermal or intramuscular injection.

Aspects also provide any of the vaccines described herein for use in a method of inducing an antigen specific immune response in a subject. In some embodiments, the method comprises administering the vaccine to the subject in an effective amount to produce an antigen specific immune response. In some embodiments, circular RNA vaccines are administered at an effective dose and using an administration schedule such that at least one symptom or feature of an infectious disease is reduced in intensity, severity, or frequency, or is delayed in onset.

Other aspects provide a use of any of the vaccines described herein in the manufacture of a medicament for use in a method of inducing an antigen specific immune response in a subject, the method comprising administering the vaccine to the subject in an effective amount to produce an antigen specific immune response.

In some embodiments, the adjuvant polypeptide comprises a toll-like receptor ligand, cytokine, FLt3-ligand, antibody, chemokines, chimeric protein, endogenous adjuvant released from a dying tumor, and checkpoint inhibition proteins. In certain embodiments, the adjuvant polypeptide is a protein that stimulates T cells, B cells, NK cells, or myeloid cell directly or indirectly. In certain embodiments, the adjuvant polypeptide increases uptake, processing, presentation of antigen peptide expression or MHC complexes on antigen presenting cells. In certain embodiment, the adjuvant polypeptide is capable of blocking MCH through down modulation.

In some embodiments, the one or more adjuvant polypeptide is selected from those proteins listed in Table 10, or an adjuvant fragment thereof. In some embodiments, the expression sequence of the one or more RNA polynucleotides and/or the expression sequence of the second RNA polynucleotide each, independently, encodes an adjuvant polypeptide selected from Table 10, or an adjuvant fragment thereof. In some embodiments, each expression sequence of the one or more RNA polynucleotides is selected from any of the RNA sequences listed in Table 10, or adjuvant fragments thereof.

In certain embodiments, provided herein is a vector for making circular RNA, the vector comprising a 5′ duplex forming region, a 3′ group I intron fragment, optionally a first spacer, an Internal Ribosome Entry Site (IRES), an expression sequence, optionally a second spacer, a 5′ group I intron fragment, and a 3′ duplex forming region. In some embodiments, these elements are positioned in the vector in the above order. In some embodiments, the vector further comprises an internal 5′ duplex forming region between the 3′ group I intron fragment and the IRES and an internal 3′ duplex forming region between the expression sequence and the 5′ group I intron fragment. In some embodiments, the internal duplex forming regions are capable of forming a duplex between each other but not with the external duplex forming regions. In some embodiments, the internal duplex forming regions are part of the first and second spacers. Additional embodiments include circular RNA polynucleotides, including circular RNA polynucleotides made using the vectors provided herein, compositions comprising such circular RNA, cells comprising such circular RNA, methods of using and making such vectors, circular RNA, compositions and cells.

In some embodiments, provided herein are methods comprising administration of circular RNA polynucleotides provided herein into cells for therapy or production of useful proteins. In some embodiments, the method is advantageous in providing the production of a desired polypeptide inside eukaryotic cells with a longer half-life than linear RNA, due to the resistance of the circular RNA to ribonucleases.

Circular RNA polynucleotides lack the free ends necessary for exonuclease-mediated degradation, causing them to be resistant to several mechanisms of RNA degradation and granting extended half-lives when compared to an equivalent linear RNA. Circularization may allow for the stabilization of RNA polynucleotides that generally suffer from short half-lives and may improve the overall efficacy of exogenous mRNA in a variety of applications. In an embodiment, the functional half-life of the circular RNA polynucleotides provided herein in eukaryotic cells (e.g., mammalian cells, such as human cells) as assessed by protein synthesis is at least 20 hours (e.g., at least 80 hours).

### Definitions

As used herein, the terms “circRNA” or “circular polyribonucleotide” or “circular RNA” are used interchangeably and refers to a polyribonucleotide that forms a circular structure through covalent bonds.

As used herein, the term “3′ group I intron fragment” refers to a sequence with 75% or higher similarity to the 3′-proximal end of a natural group I intron including the splice site dinucleotide and optionally a stretch of natural exon sequence.

As used herein, the term “5′ group I intron fragment” refers to a sequence with 75% or higher similarity to the 5′-proximal end of a natural group I intron including the splice site dinucleotide and optionally a stretch of natural exon sequence.

As used herein, the term “permutation site” refers to the site in a group I intron where a cut is made prior to permutation of the intron. This cut generates 3′ and 5′ group I intron fragments that are permuted to be on either side of a stretch of precursor RNA to be circularized.

As used herein, the term “splice site” refers to a dinucleotide that is partially or fully included in a group I intron and between which a phosphodiester bond is cleaved during RNA circularization.

The expression sequences in the polynucleotide construct may be separated by a “cleavage site” sequence which enables polypeptides encoded by the expression sequences, once translated, to be expressed as distinct and discrete separate polypeptides in the cell.

A “self-cleaving peptide” refers to a peptide which is translated without a peptide bond between two adjacent amino acids, or functions such that when the polypeptide comprising the proteins and the self-cleaving peptide is produced, it is immediately cleaved or separated into distinct and discrete first and second polypeptides without the need for any external cleavage activity (e.g., enzymatic cleavage).

As used herein, the term “therapeutic protein” refers to any protein that, when administered to a subject directly or indirectly in the form of a translated nucleic acid, has a therapeutic, diagnostic, and/or prophylactic effect and/or elicits a desired biological and/or pharmacological effect.

The α and β chains of αβ TCR's are generally regarded as each having two domains or regions, namely variable and constant domains/regions. The variable domain consists of a concatenation of variable regions and joining regions. In the present specification and claims, the term “TCR alpha variable domain” therefore refers to the concatenation of TCR alpha variable (TRAV) and TCR alpha joining (TRAJ) regions, and the term “TCR alpha constant domain” refers to the extracellular TCR alpha constant (TRAC) region, or to a C-terminal truncated TRAC sequence. Likewise the term “TCR beta variable domain” refers to the concatenation of TCR beta variable (TRBV), TCR beta diversity (TRBD), and TCR beta joining (TRBJ) regions, and the term “TCR beta constant domain” refers to the extracellular TCR beta constant (TRBC) region, or to a C-terminal truncated TRBC sequence.

As used herein, the term “immunogenic” refers to a potential to induce an immune response to a substance. An immune response may be induced when an immune system of an organism or a certain type of immune cell is exposed to an immunogenic substance. The term “non-immunogenic” refers to a lack of or absence of an immune response above a detectable threshold to a substance. No immune response is detected when an immune system of an organism or a certain type of immune cell is exposed to a non-immunogenic substance. In some embodiments, a non-immunogenic circular polyribonucleotide as provided herein, does not induce an immune response above a pre-determined threshold when measured by an immunogenicity assay. In some embodiments, no innate immune response is detected when an immune system of an organism or a certain type of immune cell is exposed to a non-immunogenic circular polyribonucleotide as provided herein. In some embodiments, no adaptive immune response is detected when an immune system of an organism or a certain type of immune cell is exposed to a non-immunogenic circular polyribonucleotide as provided herein.

As used herein, the term “circularization efficiency” refers to a measurement of resultant circular polyribonucleotide as compared to its linear starting material.

As used herein, the term “translation efficiency” refers to a rate or amount of protein or peptide production from a ribonucleotide transcript. In some embodiments, translation efficiency can be expressed as amount of protein or peptide produced per given amount of transcript that codes for the protein or peptide.

The term “nucleotide” refers to a ribonucleotide, a deoxyribonucleotide, a modified form thereof, or an analog thereof. Nucleotides include species that comprise purines, e.g., adenine, hypoxanthine, guanine, and their derivatives and analogs, as well as pyrimidines, e.g., cytosine, uracil, thymine, and their derivatives and analogs. Nucleotide analogs include nucleotides having modifications in the chemical structure of the base, sugar and/or phosphate, including, but not limited to, 5′-position pyrimidine modifications, 8′-position purine modifications, modifications at cytosine exocyclic amines, and substitution of 5-bromo-uracil; and 2′-position sugar modifications, including but not limited to, sugar-modified ribonucleotides in which the 2′-OH is replaced by a group such as an H, OR, R, halo, SH, SR, NH2, NHR, NR2, or CN, wherein R is an alkyl moiety as defined herein. Nucleotide analogs are also meant to include nucleotides with bases such as inosine, queuosine, xanthine; sugars such as 2′-methyl ribose; non-natural phosphodiester linkages such as methylphosphonate, phosphorothioate and peptide linkages. Nucleotide analogs include 5-methoxyuridine, 1-methylpseudouridine, and 6-methyladenosine.

The term “nucleic acid” and “polynucleotide” are used interchangeably herein to describe a polymer of any length, e.g., greater than about 2 bases, greater than about 10 bases, greater than about 100 bases, greater than about 500 bases, greater than 1000 bases, or up to about 10,000 or more bases, composed of nucleotides, e.g., deoxyribonucleotides or ribonucleotides, and may be produced enzymatically or synthetically (e.g., as described in U.S. Pat. No. 5,948,902 and the references cited therein), which can hybridize with naturally occurring nucleic acids in a sequence specific manner analogous to that of two naturally occurring nucleic acids, e.g., can participate in Watson-Crick base pairing interactions. Naturally occurring nucleic acids are comprised of nucleotides including guanine, cytosine, adenine, thymine, and uracil (G, C, A, T, and U respectively).

The terms “ribonucleic acid” and “RNA” as used herein mean a polymer composed of ribonucleotides.

The terms “deoxyribonucleic acid” and “DNA” as used herein mean a polymer composed of deoxyribonucleotides.

“Isolated” or “purified” generally refers to isolation of a substance (for example, in some embodiments, a compound, a polynucleotide, a protein, a polypeptide, a polynucleotide composition, or a polypeptide composition) such that the substance comprises a significant percent (e.g., greater than 1%, greater than 2%, greater than 5%, greater than 10%, greater than 20%, greater than 50%, or more, usually up to about 90%-100%) of the sample in which it resides. In certain embodiments, a substantially purified component comprises at least 50%, 80%-85%, or 90%-95% of the sample. Techniques for purifying polynucleotides and polypeptides of interest are well-known in the art and include, for example, ion-exchange chromatography, affinity chromatography and sedimentation according to density. Generally, a substance is purified when it exists in a sample in an amount, relative to other components of the sample that is more than as it is found naturally.

The terms “duplexed,” “double-stranded,” or “hybridized” as used herein refer to nucleic acids formed by hybridization of two single strands of nucleic acids containing complementary sequences. In most cases, genomic DNA is double-stranded. Sequences can be fully complementary or partially complementary.

As used herein, “unstructured” with regard to RNA refers to an RNA sequence that is not predicted by the RNAFold software or similar predictive tools to form a structure (e.g., a hairpin loop) with itself or other sequences in the same RNA molecule. In some embodiments, unstructured RNA can be functionally characterized using nuclease protection assays.

As used herein, “structured” with regard to RNA refers to an RNA sequence that is predicted by the RNAFold software or similar predictive tools to form a structure (e.g., a hairpin loop) with itself or other sequences in the same RNA molecule.

As used herein, two “duplex forming regions,” “homology arms,” or “homology regions,” complement, or are complementary, to one another when the two regions share a sufficient level of sequence identity to one another's reverse complement to act as substrates for a hybridization reaction. As used herein, polynucleotide sequences have “homology” when they are either identical or share sequence identity to a reverse complement or “complementary” sequence. The percent sequence identity between a duplex forming region and a counterpart duplex forming region's reverse complement can be any percent of sequence identity that allows for hybridization to occur. In some embodiments, an internal duplex forming region of an inventive polynucleotide is capable of forming a duplex with another internal duplex forming region and does not form a duplex with an external duplex forming region.

Linear nucleic acid molecules are said to have a “5′-terminus” (5′ end) and a “3′-terminus” (3′ end) because nucleic acid phosphodiester linkages occur at the 5′ carbon and 3′ carbon of the sugar moieties of the substituent mononucleotides. The end nucleotide of a polynucleotide at which a new linkage would be to a 5′ carbon is its 5′ terminal nucleotide. The end nucleotide of a polynucleotide at which a new linkage would be to a 3′ carbon is its 3′ terminal nucleotide. A terminal nucleotide, as used herein, is the nucleotide at the end position of the 3′- or 5′-terminus.

“Transcription” means the formation or synthesis of an RNA molecule by an RNA polymerase using a DNA molecule as a template. The invention is not limited with respect to the RNA polymerase that is used for transcription. For example, in some embodiments, a T7-type RNA polymerase can be used.

“Translation” means the formation of a polypeptide molecule by a ribosome based upon an RNA template.

It is to be understood that the terminology used herein is for the purpose of describing particular embodiments only, and is not intended to be limiting. As used in this specification and the appended claims, the singular forms “a,” “an,” and “the” include plural referents unless the content clearly dictates otherwise. Thus, for example, reference to “a cell” includes combinations of two or more cells, or entire cultures of cells; reference to “a polynucleotide” includes, as a practical matter, many copies of that polynucleotide. Unless specifically stated or obvious from context, as used herein, the term “or” is understood to be inclusive. Unless defined herein and below in the reminder of the specification, all technical and scientific terms used herein have the same meaning as commonly understood by one of ordinary skill in the art to which the invention pertains.

Unless specifically stated or obvious from context, as used herein, the term “about,” is understood as within a range of normal tolerance in the art, for example within 2 standard deviations of the mean. “About” can be understood as within 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, or 0.01% of the stated value. Unless otherwise clear from the context, all numerical values provided herein are modified by the term “about.”

As used herein, the term “encode” refers broadly to any process whereby the information in a polymeric macromolecule is used to direct the production of a second molecule that is different from the first. The second molecule may have a chemical structure that is different from the chemical nature of the first molecule.

By “co-administering” is meant administering a therapeutic agent provided herein in conjunction with one or more additional therapeutic agents sufficiently close in time such that the therapeutic agent provided herein can enhance the effect of the one or more additional therapeutic agents, or vice versa.

The terms “treat,” and “prevent” as well as words stemming therefrom, as used herein, do not necessarily imply 100% or complete treatment or prevention. Rather, there are varying degrees of treatment or prevention of which one of ordinary skill in the art recognizes as having a potential benefit or therapeutic effect. The treatment or prevention provided by the method disclosed herein can include treatment or prevention of one or more conditions or symptoms of the disease. Also, for purposes herein, “prevention” can encompass delaying the onset of the disease, or a symptom or condition thereof.

As used herein, “autoimmunity” is defined as persistent and progressive immune reactions to non-infectious self-antigens, as distinct from infectious non self-antigens from bacterial, viral, fungal, or parasitic organisms which invade and persist within mammals and humans. Autoimmune conditions include scleroderma, Grave's disease, Crohn's disease, Sjorgen's disease, multiple sclerosis, Hashimoto's disease, psoriasis, myasthenia gravis, autoimmune polyendocrinopathy syndromes, Type I diabetes mellitus (TIDM), autoimmune gastritis, autoimmune uveoretinitis, polymyositis, colitis, and thyroiditis, as well as in the generalized autoimmune diseases typified by human Lupus. “Autoantigen” or “self-antigen” as used herein refers to an antigen or epitope which is native to the mammal and which is immunogenic in said mammal.

As used herein, the term “expression sequence” can refer to a nucleic acid sequence that encodes a product, e.g., a peptide or polypeptide, regulatory nucleic acid, or non-coding nucleic acid. An exemplary expression sequence that codes for a peptide or polypeptide can comprise a plurality of nucleotide triads, each of which can code for an amino acid and is termed as a “codon”.

As used herein, a “spacer” refers to a region of a polynucleotide sequence ranging from 1 nucleotide to hundreds or thousands of nucleotides separating two other elements along a polynucleotide sequence. The sequences can be defined or can be random. A spacer is typically non-coding. In some embodiments, spacers include duplex forming regions.

As used herein, an “internal ribosome entry site” or “IRES” refers to an RNA sequence or structural element ranging in size from 10 nt to 1000 nt or more, capable of initiating translation of a polypeptide in the absence of a typical RNA cap structure. An IRES is typically about 500 nt to about 700 nt in length.

As used herein, an “miRNA site” refers to a stretch of nucleotides within a polynucleotide that is capable of forming a duplex with at least 8 nucleotides of a natural miRNA sequence.

As used herein, an “endonuclease site” refers to a stretch of nucleotides within a polynucleotide that is capable of being recognized and cleaved by an endonuclease protein.

As used herein, “bicistronic RNA” refers to a polynucleotide that includes two expression sequences coding for two distinct proteins. These expression sequences are often separated by a cleavable peptide such as a 2A site or an IRES sequence.

As used herein, the term “co-formulate” refers to a nanoparticle formulation comprising two or more nucleic acids or a nucleic acid and other active drug substance. Typically, the ratios are equimolar or defined in the ratiometric amount of the two or more nucleic acids or the nucleic acid and other active drug substance.

As used herein, “transfer vehicle” includes any of the standard pharmaceutical carriers, diluents, excipients, and the like, which are generally intended for use in connection with the administration of biologically active agents, including nucleic acids.

As used herein, the phrase “lipid nanoparticle” refers to a transfer vehicle comprising one or more lipids (e.g., in some embodiments, cationic lipids, non-cationic lipids, and PEG-modified lipids).

As used herein, the phrase “cationic lipid” refers to any of a number of lipid species that carry a net positive charge at a selected pH, such as physiological pH.

As used herein, the phrase “non-cationic lipid” refers to any neutral, zwitterionic or anionic lipid.

As used herein, the phrase “anionic lipid” refers to any of a number of lipid species that carry a net negative charge at a selected pH, such as physiological pH.

As used herein, the phrase “ionizable lipid” refers to any of a number of lipid species that carry a net positive charge at a selected pH, such as physiological pH 4 and a neutral charge at other pHs such as physiological pH 7.

The term “antibody” (Ab) includes, without limitation, a glycoprotein immunoglobulin which binds specifically to an antigen. In general, an antibody may comprise at least two heavy (H) chains and two light (L) chains interconnected by disulfide bonds, or an antigen-binding molecule thereof. Each H chain may comprise a heavy chain variable region (abbreviated herein as VH) and a heavy chain constant region. The heavy chain constant region can comprise three constant domains, CH1, CH2 and CH3. Each light chain can comprise a light chain variable region (abbreviated herein as VL) and a light chain constant region. The light chain constant region can comprise one constant domain, CL. The VH and VL regions may be further subdivided into regions of hypervariability, termed complementarity determining regions (CDRs), interspersed with regions that are more conserved, termed framework regions (FR). Each VH and VL may comprise three CDRs and four FRs, arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the Abs may mediate the binding of the immunoglobulin to host tissues or factors, including various cells of the immune system (e.g., effector cells) and the first component of the classical complement system. Antibodies may include, for example, monoclonal antibodies, recombinantly produced antibodies, monospecific antibodies, multispecific antibodies (including bispecific antibodies), human antibodies, engineered antibodies, humanized antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, antibody fusions (sometimes referred to herein as “antibody conjugates”), heteroconjugate antibodies, single domain antibodies, monovalent antibodies, single chain antibodies or single-chain variable fragments (scFv), camelized antibodies, affybodies, Fab fragments, F(ab′)₂ fragments, disulfide-linked variable fragments (sdFv), anti-idiotypic (anti-id) antibodies (including, e.g., anti-anti-Id antibodies), minibodies, domain antibodies, synthetic antibodies (sometimes referred to herein as “antibody mimetics”), and antigen-binding fragments of any of the above. In some embodiments, antibodies described herein refer to polyclonal antibody populations.

An immunoglobulin may be derived from any of the commonly known isotypes, including but not limited to IgA, secretory IgA, IgG and IgM. IgG subclasses are also well known to those in the art and include but are not limited to human IgG1, IgG2, IgG3 and IgG4. “Isotype” refers to the Ab class or subclass (e.g., IgM or IgG1) that is encoded by the heavy chain constant region genes. The term “antibody” includes, by way of example, both naturally occurring and non-naturally occurring Abs; monoclonal and polyclonal Abs; chimeric and humanized Abs; human or nonhuman Abs; wholly synthetic Abs; and single chain Abs. A nonhuman Ab may be humanized by recombinant methods to reduce its immunogenicity in humans. Where not expressly stated, and unless the context indicates otherwise, the term “antibody” also includes an antigen-binding fragment or an antigen-binding portion of any of the aforementioned immunoglobulins, and includes a monovalent and a divalent fragment or portion, and a single chain Ab.

An “antigen binding molecule,” “antigen binding portion,” or “antibody fragment” refers to any molecule that comprises the antigen binding parts (e.g., CDRs) of the antibody from which the molecule is derived. An antigen binding molecule may include the antigenic complementarity determining regions (CDRs). Examples of antibody fragments include, but are not limited to, Fab, Fab′, F(ab′)2, Fv fragments, dAb, linear antibodies, scFv antibodies, and multispecific antibodies formed from antigen binding molecules. Peptibodies (i.e. Fc fusion molecules comprising peptide binding domains) are another example of suitable antigen binding molecules. In some embodiments, the antigen binding molecule binds to an antigen on a tumor cell. In some embodiments, the antigen binding molecule binds to an antigen on a cell involved in a hyperproliferative disease or to a viral or bacterial antigen. In some embodiments, the antigen binding molecule binds to BCMA. In further embodiments, the antigen binding molecule is an antibody fragment, including one or more of the complementarity determining regions (CDRs) thereof, that specifically binds to the antigen. In further embodiments, the antigen binding molecule is a single chain variable fragment (scFv). In some embodiments, the antigen binding molecule comprises or consists of avimers.

As used herein, the term “variable region” or “variable domain” is used interchangeably and are common in the art. The variable region typically refers to a portion of an antibody, generally, a portion of a light or heavy chain, typically about the amino-terminal 110 to 120 amino acids in the mature heavy chain and about 90 to 115 amino acids in the mature light chain, which differ extensively in sequence among antibodies and are used in the binding and specificity of a particular antibody for its particular antigen. The variability in sequence is concentrated in those regions called complementarity determining regions (CDRs) while the more highly conserved regions in the variable domain are called framework regions (FR). Without wishing to be bound by any particular mechanism or theory, it is believed that the CDRs of the light and heavy chains are primarily responsible for the interaction and specificity of the antibody with antigen. In some embodiments, the variable region is a human variable region. In some embodiments, the variable region comprises rodent or murine CDRs and human framework regions (FRs). In particular embodiments, the variable region is a primate (e.g., non-human primate) variable region. In some embodiments, the variable region comprises rodent or murine CDRs and primate (e.g., non-human primate) framework regions (FRs).

The terms “VL” and “VL domain” are used interchangeably to refer to the light chain variable region of an antibody or an antigen-binding molecule thereof.

The terms “VH” and “VH domain” are used interchangeably to refer to the heavy chain variable region of an antibody or an antigen-binding molecule thereof.

A number of definitions of the CDRs are commonly in use: Kabat numbering, Chothia numbering, AbM numbering, or contact numbering. The AbM definition is a compromise between the two used by Oxford Molecular's AbM antibody modelling software. The contact definition is based on an analysis of the available complex crystal structures. The term “Kabat numbering” and like terms are recognized in the art and refer to a system of numbering amino acid residues in the heavy and light chain variable regions of an antibody, or an antigen-binding molecule thereof. In certain aspects, the CDRs of an antibody may be determined according to the Kabat numbering system (see, e.g., Kabat EA & Wu TT (1971) Ann NY Acad Sci 190: 382-391 and Kabat E A et al., (1991) Sequences of Proteins of Immunological Interest, Fifth Edition, U.S. Department of Health and Human Services, NIH Publication No. 91-3242). Using the Kabat numbering system, CDRs within an antibody heavy chain molecule are typically present at amino acid positions 31 to 35, which optionally may include one or two additional amino acids, following 35 (referred to in the Kabat numbering scheme as 35A and 35B) (CDR1), amino acid positions 50 to 65 (CDR2), and amino acid positions 95 to 102 (CDR3). Using the Kabat numbering system, CDRs within an antibody light chain molecule are typically present at amino acid positions 24 to 34 (CDR1), amino acid positions 50 to 56 (CDR2), and amino acid positions 89 to 97 (CDR3). In a specific embodiment, the CDRs of the antibodies described herein have been determined according to the Kabat numbering scheme. In certain aspects, the CDRs of an antibody may be determined according to the Chothia numbering scheme, which refers to the location of immunoglobulin structural loops (see, e.g., Chothia C & Lesk AM, (1987), J Mol Biol 196: 901-917; Al-Lazikani B et al., (1997) J Mol Biol 273: 927-948; Chothia C el al., (1992) J Mol Biol 227: 799-817; Tramontano A et al., (1990) J Mol Biol 215(1): 175-82; and U.S. Pat. No. 7,709,226). Typically, when using the Kabat numbering convention, the Chothia CDR-H1 loop is present at heavy chain amino acids 26 to 32, 33, or 34, the Chothia CDR-H2 loop is present at heavy chain amino acids 52 to 56, and the Chothia CDR-H3 loop is present at heavy chain amino acids 95 to 102, while the Chothia CDR-L1 loop is present at light chain amino acids 24 to 34, the Chothia CDR-L2 loop is present at light chain amino acids 50 to 56, and the Chothia CDR-L3 loop is present at light chain amino acids 89 to 97. The end of the Chothia CDR-HI loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33: if both 35A and 35B are present, the loop ends at 34). In a specific embodiment, the CDRs of the antibodies described herein have been determined according to the Chothia numbering scheme.

As used herein, the terms “constant region” and “constant domain” are interchangeable and have a meaning commonly understood in the art. The constant region is an antibody portion, e.g., a carboxyl terminal portion of a light and/or heavy chain which is not directly involved in binding of an antibody to antigen but which may exhibit various effector functions, such as interaction with the Fc receptor. The constant region of an immunoglobulin molecule generally has a more conserved amino acid sequence relative to an immunoglobulin variable domain.

“Binding affinity” generally refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y may generally be represented by a dissociation constant (KD or Kd). Affinity may be measured and/or expressed in a number of ways known in the art, including, but not limited to, equilibrium dissociation constant (KD), and equilibrium association constant (KA or Ka). The KD is calculated from the quotient of koff/kon, whereas KA is calculated from the quotient of kon/koff. kon refers to the association rate constant of, e.g., an antibody to an antigen, and koff refers to the dissociation of, e.g., an antibody to an antigen. The kon and koff may be determined by techniques known to one of ordinary skill in the art, such as BIACORE® or KinExA.

As used herein, a “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). In some embodiments, one or more amino acid residues within a CDR(s) or within a framework region(s) of an antibody or antigen-binding molecule thereof may be replaced with an amino acid residue with a similar side chain.

As, used herein, the term “heterologous sequence” means an exogenous sequence that is not native or naturally present in a cell, or organism expressing the sequence.

As used herein, an “epitope” is a term in the art and refers to a localized region of an antigen to which an antibody may specifically bind. An epitope may be, for example, contiguous amino acids of a polypeptide (linear or contiguous epitope) or an epitope can, for example, come together from two or more non-contiguous regions of a polypeptide or polypeptides (conformational, non-linear, discontinuous, or non-contiguous epitope). In some embodiments, the epitope to which an antibody binds may be determined by, e.g., NMR spectroscopy, X-ray diffraction crystallography studies, ELISA assays, hydrogen/deuterium exchange coupled with mass spectrometry (e.g., liquid chromatography electrospray mass spectrometry), array-based oligo-peptide scanning assays, and/or mutagenesis mapping (e.g., site-directed mutagenesis mapping). For X-ray crystallography, crystallization may be accomplished using any of the known methods in the art (e.g., Giege R et al., (1994) Acta Crystallogr D Biol Crystallogr 50(Pt 4): 339-350; McPherson A (1990) Eur J Biochem 189:1-23; Chayen N E (1997) Structure 5:1269-1274; McPherson A (1976) J Biol Chem 251: 6300-6303). Antibody: antigen crystals may be studied using well known X-ray diffraction techniques and may be refined using computer software such as X-PLOR (Yale University, 1992, distributed by Molecular Simulations, Inc.; see e.g. Meth Enzymol (1985) volumes 114 & 115, eds Wyckoff H W et al.; U.S. Patent Publication No. 2004/0014194), and BUSTER (Bricogne G (1993) Acta Crystallogr D Biol Crystallogr 49(Pt 1): 37-60; Bricogne G (1997) Meth Enzymol 276A: 361-423, ed Carter C W; Roversi P et al., (2000) Acta Crystallogr D Biol Crystallogr 56(Pt 10): 1316-1323).

As used herein, an antigen binding molecule, an antibody, or an antigen binding fragment thereof “cross-competes” with a reference antibody or a reference antigen binding fragment thereof if the interaction between an antigen and the first binding molecule, an antibody, or an antigen binding fragment thereof blocks, limits, inhibits, or otherwise reduces the ability of the reference binding molecule, reference antibody, or a reference antigen binding fragment thereof to interact with the antigen. Cross competition may be complete, e.g., binding of the binding molecule to the antigen completely blocks the ability of the reference binding molecule to bind the antigen, or it may be partial, e.g., binding of the binding molecule to the antigen reduces the ability of the reference binding molecule to bind the antigen. In some embodiments, an antigen binding molecule that cross-competes with a reference antigen binding molecule binds the same or an overlapping epitope as the reference antigen binding molecule. In other embodiments, the antigen binding molecule that cross-competes with a reference antigen binding molecule binds a different epitope as the reference antigen binding molecule. Numerous types of competitive binding assays may be used to determine if one antigen binding molecule competes with another, for example: solid phase direct or indirect radioimmunoassay (RIA); solid phase direct or indirect enzyme immunoassay (EIA); sandwich competition assay (Stahli et al., 1983, Methods in Enzymology 9:242-253); solid phase direct biotin-avidin EIA (Kirkland el al., 1986, J. Immunol. 137:3614-3619); solid phase direct labeled assay, solid phase direct labeled sandwich assay (Harlow and Lane, 1988, Antibodies, A Laboratory Manual, Cold Spring Harbor Press); solid phase direct label RIA using 1-125 label (Morel et al., 1988, Molec. Immunol. 25:7-15); solid phase direct biotin-avidin EIA (Cheung, et al., 1990, Virology 176:546-552); and direct labeled RIA (Moldenhauer et al., 1990, Scand. J. Immunol. 32:77-82).

As used herein, the terms “immunospecifically binds,” “immunospecifically recognizes,” “specifically binds,” and “specifically recognizes” are analogous terms in the context of antibodies and refer to molecules that bind to an antigen (e.g., epitope or immune complex) as such binding is understood by one skilled in the art. For example, a molecule that specifically binds to an antigen may bind to other peptides or polypeptides, generally with lower affinity as determined by, e.g., immunoassays, BIACORE®, KinExA 3000 instrument (Sapidyne Instruments, Boise, ID), or other assays known in the art. In a specific embodiment, molecules that specifically bind to an antigen bind to the antigen with a KA that is at least 2 logs, 2.5 logs, 3 logs, 4 logs or greater than the KA when the molecules bind to another antigen.

As defined herein, the term “antigen” refers to a molecule that binds to an antigen binding molecule, an antibody, or an antigen binding fragment thereof. For example, an antigen can elicit an innate or adaptive immune response in an organism. Antigens can be any immunogenic substance including, in particular, proteins, polypeptides, polysaccharides, nucleic acids, lipids and the like. In some embodiments, antigens are derived from infectious agents.

The term “autologous” refers to any material derived from the same individual to which the material is then later re-introduced. For example, the engineered autologous cell therapy (eACT™) method described herein involves collection of lymphocytes from a patient, which are then engineered to express, e.g., a CAR construct, and then administered back to the same patient.

The term “allogeneic” refers to any material derived from one individual which is then introduced to another individual of the same species, e.g., allogeneic T cell transplantation.

A “cytokine,” as used herein, refers to a non-antibody protein that is released by one cell and can interact with a second cell to mediate a response in the second cell. “Cytokine” as used herein is meant to refer to proteins released by one cell population that act on another cell as intercellular mediators. A cytokine may be endogenously expressed by a cell or administered to a subject. Cytokines may be released by immune cells, including, but not limited to, macrophages, B cells, T cells, neutrophils, dendritic cells, eosinophils and mast cells to propagate an immune response. Cytokines may induce various cellular responses. Cytokines may include homeostatic cytokines, chemokines, pro-inflammatory cytokines, effector cytokines, and acute-phase proteins. For example, homeostatic cytokines, including interleukin (IL) 7 and IL-15, promote immune cell survival and proliferation, and pro-inflammatory cytokines may promote an inflammatory response. Examples of homeostatic cytokines include, but are not limited to, IL-2, IL-4, IL-5, IL-7, IL-10, IL-12p40, IL-12p70, IL-15, and interferon (IFN) gamma. Examples of pro-inflammatory cytokines include, but are not limited to, IL-la, IL-1b, IL-6, IL-13, IL-17a, IL-23, IL-27, tumor necrosis factor (TNF)-alpha, TNF-beta, fibroblast growth factor (FGF) 2, granulocyte macrophage colony-stimulating factor (GM-CSF), soluble intercellular adhesion molecule 1 (sICAM-1), soluble vascular adhesion molecule 1 (sVCAM-1), vascular endothelial growth factor (VEGF), VEGF-C, VEGF-D, and placental growth factor (PLGF). Examples of effector cytokines include, but are not limited to, granzyme A, granzyme B, soluble Fas ligand (sFasL), TGF-beta, IL-35, and perforin. Examples of acute phase-proteins include, but are not limited to, C-reactive protein (CRP) and serum amyloid A (SAA).

The term “lymphocyte” as used herein includes natural killer (NK) cells, T cells, or B cells. NK cells are a type of cytotoxic (cell toxic) lymphocyte that represent a major component of the innate immune system. NK cells can induce apoptosis of tumors and virally-infected cells. They were termed “natural killers” because they do not require activation in order to kill target cells. T cells play a major role in cell-mediated-immunity (no antibody involvement). T cell receptors (TCR) differentiate T cells from other lymphocyte types. The thymus, a specialized organ of the immune system, is the primary site for T cell maturation. There are numerous types of T cells, including: helper T cells (e.g., CD4+ cells), cytotoxic T cells (also known as TC, cytotoxic T lymphocytes, CTL, T-killer cells, cytolytic T cells, CD8+ T cells or killer T cells), memory T cells ((i) stem memory cells (TSCM), like naive cells, are CD45RO−, CCR7+, CD45RA+, CD62L+ (L-selectin), CD27+, CD28+ and IL-7Ra+, but also express large amounts of CD95, IL-2R, CXCR3, and LFA-1, and show numerous functional attributes distinctive of memory cells); (ii) central memory cells (TCM) express L-selectin and CCR7, they secrete IL-2, but not IFNγ or IL-4, and (iii) effector memory cells (TEM), however, do not express L-selectin or CCR7 but produce effector cytokines like IFNγ and IL-4), regulatory T cells (Tregs, suppressor T cells, or CD4+CD25+ or CD4+FoxP3+ regulatory T cells), natural killer T cells (NKT) and gamma delta T cells. B-cells, on the other hand, play a principal role in humoral immunity (with antibody involvement). B-cells make antibodies, are capable of acting as antigen-presenting cells (APCs) and turn into memory B-cells and plasma cells, both short-lived and long-lived, after activation by antigen interaction. In mammals, immature B-cells are formed in the bone marrow.

The term “genetically engineered” or “engineered” refers to a method of modifying the genome of a cell, including, but not limited to, deleting a coding or non-coding region or a portion thereof or inserting a coding region or a portion thereof. In some embodiments, the cell that is modified is a lymphocyte, e.g., a T cell, which may either be obtained from a patient or a donor. The cell may be modified to express an exogenous construct, such as, e.g., a chimeric antigen receptor (CAR) or a T cell receptor (TCR), which is incorporated into the cell's genome.

An “immune response” refers to the action of a cell of the immune system (for example, T lymphocytes, B lymphocytes, natural killer (NK) cells, macrophages, eosinophils, mast cells, dendritic cells and neutrophils) and soluble molecules produced by any of these cells or the liver (including Abs, cytokines, and complement) that results in selective targeting, binding to, damage to, destruction of, and/or elimination from a vertebrate's body of invading pathogens, cells or tissues infected with pathogens, cancerous or other abnormal cells, or, in cases of autoimmunity or pathological inflammation, normal human cells or tissues.

The term “sequence identity,” as used herein, refers to the extent that sequences are identical on a nucleotide-by-nucleotide basis or an amino acid-by-amino acid basis over a window of comparison. Thus, a “percentage of sequence identity” may be calculated by comparing two optimally aligned sequences over the window of comparison, determining the number of positions at which the identical nucleic acid base (e.g., A, T, C, G, U) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e. the window size), and multiplying the result by 100 to yield the percentage of sequence identity. Included are nucleotides and polypeptides having at least about 50%, 55%, 60%, 65%, 70%, 75%, 80%, 85%, 90%, 95%, 96%, 97%, 98%, 99% or 100% sequence identity to any of the reference sequences described herein, typically where, in the case of polypeptides, the polypeptide variant maintains at least one biological activity of the polypeptide encoded by the reference sequence.

As used herein, an “adjuvant” refers to a drug or substance that modulates the immunogenicity of an antigen.

As used herein, a “vaccine” refers to a composition, for example, a substance or preparation that stimulates, induces, causes or improves immunity in an organism, e.g., an animal organism, for example, a mammalian organism (e.g., a human). In some embodiments, a vaccine provides immunity against one or more diseases or disorders in the organism, including prophylactic and/or therapeutic immunity. In some embodiments, vaccines can be made, for example, from live, attenuated, modified, weakened or killed forms of disease-causing microorganisms, or antigens derived therefrom, including combinations of antigenic components. In some embodiments, a vaccine stimulates, induces, causes or improves immunity in an organism or causes or mimics an immune response in the organism without inducing any disease or disorder. In some embodiments, a vaccine elicits an immune response after being introduced into the tissues, extracellular space or cells of a subject. In some embodiments, polynucleotides of the present invention may encode an antigen and when the polynucleotides are expressed in cells, the expressed antigen elicits a desired immune response.

### Vectors, Precursor RNA, and Circular RNA

In certain aspects, provided herein are circular RNA polynucleotides comprising a post splicing 3′ group I intron fragment, optionally a first spacer, an Internal Ribosome Entry Site (IRES), an expression sequence, optionally a second spacer, and a post splicing 5′ group I intron fragment. In some embodiments, these regions are in that order. In some embodiments, the circular RNA is made by a method provided herein or from a vector provided herein.

In certain embodiments, transcription of a vector provided herein (e.g., comprising a 5′ duplex forming region, a 3′ group I intron fragment, optionally a first spacer, an Internal Ribosome Entry Site (IRES), a first expression sequence, a polynucleotide sequence encoding a cleavage site, a second expression sequence, optionally a second spacer, a 5′ group I intron fragment, and a 3′ duplex forming region) results in the formation of a precursor linear RNA polynucleotide capable of circularizing. In some embodiments, this precursor linear RNA polynucleotide circularizes when incubated in the presence of guanosine nucleotide or nucleoside (e.g., GTP) and divalent cation (e.g., Mg2+).

In some embodiments, the vectors and precursor RNA polynucleotides provided herein comprise a first (5′) duplex forming region and a second (3′) duplex forming region. In certain embodiments, the first and second duplex forming regions may form perfect or imperfect duplexes. Thus, in certain embodiments at least 75%, 80%, 85%, 90/o, 91%, 92%, 93%, 94%, 95%, 96%, 97%, 98%, 99% or 100% of the first and second duplex forming regions may be base paired with one another. In some embodiments, the duplex forming regions are predicted to have less than 50% (e.g., less than 45%, less than 40%, less than 35%, less than 30%, less than 25%) base pairing with unintended sequences in the RNA (e.g., non-duplex forming region sequences). In some embodiments, including such duplex forming regions on the ends of the precursor RNA strand, and adjacent or very close to the group I intron fragment, bring the group I intron fragments in close proximity to each other, increasing splicing efficiency. In some embodiments, the duplex forming regions are 3 to 100 nucleotides in length (e.g., 3-75 nucleotides in length, 3-50 nucleotides in length, 20-50 nucleotides in length, 35-50 nucleotides in length, 5-25 nucleotides in length, 9-19 nucleotides in length). In some embodiments, the duplex forming regions are about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides in length. In some embodiments, the duplex forming regions have a length of about 9 to about 50 nucleotides. In one embodiment, the duplex forming regions have a length of about 9 to about 19 nucleotides. In some embodiments, the duplex forming regions have a length of about 20 to about 40 nucleotides. In certain embodiments, the duplex forming regions have a length of about 30 nucleotides.

In certain embodiments, the vectors, precursor RNA and circular RNA provided herein comprise a first (5′) and/or a second (3′) spacer. In some embodiments, including a spacer between the 3′ group I intron fragment and the IRES may conserve secondary structures in those regions by preventing them from interacting, thus increasing splicing efficiency. In some embodiments, the first (between 3′ group I intron fragment and IRES) and second (between the expression sequences and 5′ group I intron fragment) spacers comprise additional base pairing regions that are predicted to base pair with each other and not to the first and second duplex forming regions. In some embodiments, such spacer base pairing brings the group I intron fragments in close proximity to each other, further increasing splicing efficiency. Additionally, in some embodiments, the combination of base pairing between the first and second duplex forming regions, and separately, base pairing between the first and second spacers, promotes the formation of a splicing bubble containing the group I intron fragments flanked by adjacent regions of base pairing. Typical spacers are contiguous sequences with one or more of the following qualities: 1) predicted to avoid interfering with proximal structures, for example, the IRES, expression sequences, or intron; 2) is at least 7 nt long and no longer than 100 nt; 3) is located after and adjacent to the 3′ intron fragment and/or before and adjacent to the 5′ intron fragment; and 4) contains one or more of the following: a) an unstructured region at least 5 nt long, b) a region of base pairing at least 5 nt long to a distal sequence, including another spacer, and c) a structured region at least 7 nt long limited in scope to the sequence of the spacer. Spacers may have several regions, including an unstructured region, a base pairing region, a hairpin/structured region, and combinations thereof. In an embodiment, the spacer has a structured region with high GC content. In an embodiment, a region within a spacer base pairs with another region within the same spacer. In an embodiment, a region within a spacer base pairs with a region within another spacer. In an embodiment, a spacer comprises one or more hairpin structures. In an embodiment, a spacer comprises one or more hairpin structures with a stem of 4 to 12 nucleotides and a loop of 2 to 10 nucleotides. In an embodiment, there is an additional spacer between the 3′ group I intron fragment and the IRES. In an embodiment, this additional spacer prevents the structured regions of the IRES from interfering with the folding of the 3′ group I intron fragment or reduces the extent to which this occurs. In some embodiments, the 5′ spacer sequence is at least 7, 8, 9, 10, 11, 12, 13, 14, 15, 16, 17, 18, 19, 20, 25 or 30 nucleotides in length. In some embodiments, the 5′ spacer sequence is no more than 100, 90, 80, 70, 60, 50, 45, 40, 35 or 30 nucleotides in length. In some embodiments the 5′ spacer sequence is between 5 and 50, 10 and 50, 20 and 50, 20 and 40, and/or 25 and 35 nucleotides in length. In certain embodiments, the 5′ spacer sequence is 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides in length. In one embodiment, the 5′ spacer sequence is a polyA sequence. In another embodiment, the 5′ spacer sequence is a polyAC sequence. In one embodiment, a spacer comprises about 10%, 20%, 30%, 40%, 50%, 60%, 70%, 80%, 90%, or 100% polyAC content. In one embodiment, a spacer comprises about 10%, 20%, 30%, 40%, 50%, 60%, 70%, 80%, 90%, or 100% polypyrimidine (C/T or C/U) content.

In certain embodiments, a 3′ group I intron fragment is a contiguous sequence at least 75% homologous (e.g., at least 80%, 85%, 90%, 91%, 92%, 93%, 94%, 95%, 96%, 97%, 98%, 99% or 100% homologous) to a 3′ proximal fragment of a natural group I intron including the 3′ splice site dinucleotide and optionally the adjacent exon sequence at least 1 nt in length (e.g., at least 2, 3, 4, 5, 6, 7, 8, 9, 10, 15, 20, 25 or 30 nt in length) and at most the length of the exon. Typically, a 5′ group I intron fragment is a contiguous sequence at least 75% homologous (e.g., at least 80%, 85%, 90%, 91%, 92%, 93%, 94%, 95%, 96%, 97%, 98%, 99% or 100% homologous) to a 5′ proximal fragment of a natural group I intron including the 5′ splice site dinucleotide and optionally the adjacent exon sequence at least 1 nt in length (e.g., at least 2, 3, 4, 5, 6, 7, 8, 9, 10, 15, 20, 25 or 30 nt in length) and at most the length of the exon. As described by Umekage et al. (2012), external portions of the 3′ group I intron fragment and 5′ group I intron fragment are removed in circularization, causing the circular RNA provided herein to comprise only the portion of the 3′ group I intron fragment formed by the optional exon sequence of at least 1 nt in length and 5′ group I intron fragment formed by the optional exon sequence of at least 1 nt in length, if such sequences were present on the non-circularized precursor RNA. The part of the 3′ group I intron fragment that is retained by a circular RNA is referred to herein as the “post splicing 3′ group I intron fragment”. The part of the 5′ group I intron fragment that is retained by a circular RNA is referred to herein as the “post splicing 5′ group I intron fragment”.

In certain embodiments, the vectors, precursor RNA and circular RNA provided herein comprise an internal ribosome entry site (IRES). Inclusion of an IRES permits the translation of one or more open reading frames from a circular RNA (e.g., open reading frames that form the expression sequences). The IRES element attracts a eukaryotic ribosomal translation initiation complex and promotes translation initiation. See, e.g., Kaufman et al., Nuc. Acids Res. (1991) 19:4485-4490; Gurtu et al., Biochem. Biophys. Res. Comm. (1996) 229:295-298: Rees et al., BioTechniques (1996) 20:102-110; Kobayashi et al., BioTechniques (1996) 21:399-402; and Mosser et al., BioTechniques 1997 22 150-161.

A multitude of IRES sequences are available and include sequences derived from a wide variety of viruses, such as from leader sequences of picornaviruses such as the encephalomyocarditis virus (EMCV) UTR (Jang et al., J. Virol. (1989) 63:1651-1660), the polio leader sequence, the hepatitis A virus leader, the hepatitis C virus IRES, human rhinovirus type 2 IRES (Dobrikova et al., Proc. Natl. Acad. Sci. (2003) 100(25): 15125-15130), an IRES element from the foot and mouth disease virus (Ramesh et al., Nucl. Acid Res. (1996) 24:2697-2700), a giardiavirus IRES (Garlapati et al., J. Biol. Chem. (2004) 279(5):3389-3397), and the like.

In some embodiments, an IRES is an IRES sequence of Taura syndrome virus, Triatoma virus, Theiler's encephalomyelitis virus, Simian Virus 40, Solenopsis invicta virus 1, Rhopalosiphum padi virus, Reticuloendotheliosis virus, Human poliovirus 1, Plautia stali intestine virus, Kashmir bee virus, Human rhinovirus 2, Homalodisca coagulata virus-1, Human Immunodeficiency Virus type 1, Himetobi P virus, Hepatitis C virus, Hepatitis A virus, Hepatitis GB virus, Foot and mouth disease virus, Human enterovirus 71, Equine rhinitis virus, Ectropis obliqua picorna-like virus, Encephalomyocarditis virus, Drosophila C Virus, Human coxsackievirus B3, Crucifer tobamovirus, Cricket paralysis virus, Bovine viral diarrhea virus 1, Black Queen Cell Virus, Aphid lethal paralysis virus, Avian encephalomyelitis virus, Acute bee paralysis virus, Hibiscus chlorotic ringspot virus, Classical swine fever virus, Human FGF2, Human SFTPA1, Human AML1/RUNX1, Drosophila antennapedia, Human AQP4, Human AT1R, Human BAG-1, Human BCL2, Human BiP, Human c-IAPl, Human c-myc, Human eIF4G, Mouse NDST4L, Human LEF1, Mouse HIF1 alpha, Human n.myc, Mouse Gtx, Human p27kipl, Human PDGF2/c-sis, Human p53, Human Pim-1, Mouse Rbm3, Drosophila reaper, Canine Scamper, Drosophila Ubx, Human UNR, Mouse UtrA, Human VEGF-A, Human XIAP, Drosophila hairless, S. cerevisiae TFIID, S. cerevisiae YAP1, tobacco etch virus, turnip crinkle virus, EMCV-A, EMCV-B, EMCV-Bf, EMCV-Cf, EMCV pEC9, Picobirnavirus, HCV QC64, Human Cosavirus E/D, Human Cosavirus F, Human Cosavirus JMY, Rhinovirus NAT001, HRV14, HRV89, HRVC-02, HRV-A21, Salivirus A SH1, Salivirus FHB, Salivirus NG-J1, Human Parechovirus 1, Crohivirus B, Yc-3, Rosavirus M-7, Shanbavirus A, Pasivirus A, Pasivirus A 2, Echovirus E14, Human Parechovirus 5, Aichi Virus, Hepatitis A Virus HA16, Phopivirus, CVA10, Enterovirus C, Enterovirus D, Enterovirus J, Human Pegivirus 2, GBV-C GT110, GBV-C K1737, GBV-C Iowa, Pegivirus A 1220, Pasivirus A 3, Sapelovirus, Rosavirus B, Bakunsa Virus, Tremovirus A, Swine Pasivirus 1, PLV-CHN, Pasivirus A, Sicinivirus, Hepacivirus K, Hepacivirus A, BVDV1, Border Disease Virus, BVDV2, CSFV-PK15C, SF573 Dicistrovirus, Hubei Picorna-like Virus, CRPV, Salivirus A BN5, Salivirus A BN2, Salivirus A 02394, Salivirus A GUT, Salivirus A CH, Salivirus A SZ1, Salivirus FHB, CVB3, CVB1, Echovirus 7, CVB5, EVA71, CVA3, CVA12, EV24 or an aptamer to eIF4G.

In some embodiments, the polynucleotides herein comprise more than one expression sequence.

In certain embodiments, the vectors provided herein comprise a 3′ UTR. In some embodiments, the 3′ UTR is from human beta globin, human alpha globin xenopus beta globin, xenopus alpha globin, human prolactin, human GAP-43, human eEFlal, human Tau, human TNFα, dengue virus, hantavirus small mRNA, bunyavirus small mRNA, turnip yellow mosaic virus, hepatitis C virus, rubella virus, tobacco mosaic virus, human IL-8, human actin, human GAPDH, human tubulin, hibiscus chlorotic ringspot virus, woodchuck hepatitis virus post translationally regulated element, sindbis virus, turnip crinkle virus, tobacco etch virus, or Venezuelan equine encephalitis virus.

In some embodiments, the vectors provided herein comprise a 5′ UTR. In some embodiments, the 5′ UTR is from human beta globin, Xenopus laevis beta globin, human alpha globin, Xenopus laevis alpha globin, rubella virus, tobacco mosaic virus, mouse Gtx, dengue virus, heat shock protein 70 kDa protein IA, tobacco alcohol dehydrogenase, tobacco etch virus, turnip crinkle virus, or the adenovirus tripartite leader.

In some embodiments, the vector provided herein comprises a polyA region. In some embodiments the polyA region is at least 12 nucleotides long, at least 30 nucleotides long or at least 60 nucleotides long.

In some embodiments, the DNA (e.g., vector), linear RNA (e.g., precursor RNA), and/or circular RNA polynucleotide provided herein is between 300 and 15000, 300 and 14000, 300 and 13000, 300 and 12000, 300 and 11000, 300 and 10000, 400 and 9000, 500 and 8000, 600 and 7000, 700 and 6000, 800 and 5000, 900 and 5000, 1000 and 5000, 1100 and 5000, 1200 and 5000, 1300 and 5000, 1400 and 5000, and/or 1500 and 5000 nucleotides in length. In some embodiments, the polynucleotide is at least 300 nt, 400 nt, 500 nt, 600 nt, 700 nt, 800 nt, 900 nt, 1000 nt, 1100 nt, 1200 nt, 1300 nt, 1400 nt, 1500 nt, 2000 nt, 2500 nt, 3000 nt, 3500 nt, 4000 nt, 4500 nt, 5000 nt, 6000 nt, 7000 nt, 8000 nt, 9000 nt, 10000 nt, 11000 nt, 12000 nt, 13000 nt, 14000 nt, or 15000 nt in length. In some embodiments, the polynucleotide is no more than 3000 nt, 3500 nt, 4000 nt, 4500 nt, 5000 nt, 6000 nt, 7000 nt, 8000 nt, 9000 nt, 10000 nt, 11000 nt, 12000 nt, 13000 nt, 14000 nt, 15000 nt, or 16000 nt in length. In some embodiments, the length of a DNA, linear RNA, and/or circular RNA polynucleotide provided herein is about 300 nt, 400 nt, 500 nt, 600 nt, 700 nt, 800 nt, 900 nt, 1000 nt, 1100 nt, 1200 nt, 1300 nt, 1400 nt, 1500 nt, 2000 nt, 2500 nt, 3000 nt, 3500 nt, 4000 nt, 4500 nt, 5000 nt, 6000 nt, 7000 nt, 8000 nt, 9000 nt, 10000 nt, 11000 nt, 12000 nt, 13000 nt, 14000 nt, or 15000 nt.

In some embodiments, provided herein is a vector. In certain embodiments, the vector comprises, in the following order, a) a 5′ duplex forming region, b) a 3′ group I intron fragment, c) optionally, a first spacer sequence, d) an IRES, e) a first expression sequence, f) a polynucleotide sequence encoding a cleavage site, g) a second expression sequence, h) optionally, a second spacer sequence, i) a 5′ group I intron fragment, and j) a 3′ duplex forming region. In some embodiments, the vector comprises a transcriptional promoter upstream of the 5′ duplex forming region.

In some embodiments, provided herein is a vector. In certain embodiments, the vector comprises, in the following order, a) a 5′ duplex forming region, b) a 3′ group I intron fragment, c) optionally, a first spacer sequence, d) a first IRES, e) a first expression sequence, f) a second IRES, g) a second expression sequence, h) optionally, a second spacer sequence, i) a 5′ group I intron fragment, and j) a 3′ duplex forming region. In some embodiments, the vector comprises a transcriptional promoter upstream of the 5′ duplex forming region.

In some embodiments, provided herein is a precursor RNA. In certain embodiments, the precursor RNA is a linear RNA produced by in vitro transcription of a vector provided herein. In some embodiments, the precursor RNA comprises, in the following order, a) optionally, a 5′ duplex forming region, b) a 3′ group I intron fragment, c) optionally, a first spacer sequence, d) an IRES, e) a first expression sequence, f) a polynucleotide sequence encoding a cleavage site, g) a second expression sequence, h) optionally, a second spacer sequence, i) a 5′ group I intron fragment, and j) optionally, a 3′ duplex forming region. In some embodiments, the precursor RNA comprises, in the following order, a) a 5′ duplex forming region, b) a 3′ group I intron fragment, c) optionally, a first spacer sequence, d) a first IRES, e) a first expression sequence, f) a second IRES, g) a second expression sequence, h) optionally, a second spacer sequence, i) a 5′ group I intron fragment, and j) a 3′ duplex forming region. The precursor RNA can be unmodified, partially modified or completely modified.

In certain embodiments, provided herein is a circular RNA. In certain embodiments, the circular RNA is a circular RNA produced by a vector provided herein. In some embodiments, the circular RNA is circular RNA produced by circularization of a precursor RNA provided herein. In some embodiments, the circular RNA comprises, in the following sequence, a) a first spacer sequence, b) an IRES, c) a first expression sequence, d) a polynucleotide sequence encoding a cleavage site, e) a second expression sequence, and f) a second spacer sequence. In some embodiments, the circular RNA comprises, in the following sequence, a) a post splicing 3′ group I intron fragment, b) a first spacer sequence, c) an IRES, d) a first expression sequence, e) a polynucleotide sequence encoding a cleavage site, f) a second expression sequence, and g) a second spacer sequence, h) a post splicing 5′ group I intron fragment. In some embodiments, the circular RNA comprises, in the following sequence, a) a first spacer sequence, b) a first IRES, c) a first expression sequence, d) a second IRES, e) a second expression sequence, and f) a second spacer sequence. In some embodiments, the circular RNA further comprises the portion of the 3′ group I intron fragment that is 3′ of the 3′ splice site. In some embodiments, the circular RNA further comprises the portion of the 5′ group I intron fragment that is 5′ of the 5′ splice site. In some embodiments, the circular RNA is at least 500, 600, 700, 800, 900, 1000, 1500, 2000, 2500, 3000, 3500, 4000, 4500, 5000, 6000, 7000, 8000, 9000, 10000, 11000, 12000, 13000, 14000, or 15000 nucleotides in size. The circular RNA can be unmodified, partially modified or completely modified.

In some embodiments, the circular RNA provided herein has higher functional stability than mRNA comprising the same expression sequence. In some embodiments, the circular RNA provided herein has higher functional stability than mRNA comprising the same expression sequence, 5 moU modifications, an optimized UTR, a cap, and/or a polyA tail.

In some embodiments, the circular RNA polynucleotide provided herein has a functional half-life of at least 5 hours, 10 hours, 15 hours, 20 hours. 30 hours, 40 hours, 50 hours, 60 hours, 70 hours or 80 hours. In some embodiments, the circular RNA polynucleotide provided herein has a functional half-life of 5-80, 10-70, 15-60, and/or 20-50 hours. In some embodiments, the circular RNA polynucleotide provided herein has a functional half-life greater than (e.g., at least 1.5-fold greater than, at least 2-fold greater than) that of an equivalent linear RNA polynucleotide encoding the same protein. In some embodiments, functional half-life can be assessed through the detection of functional protein synthesis.

In some embodiments, the circular RNA polynucleotide provided herein has a half-life of at least 5 hours, 10 hours, 15 hours, 20 hours. 30 hours, 40 hours, 50 hours, 60 hours, 70 hours or 80 hours. In some embodiments, the circular RNA polynucleotide provided herein has a half-life of 5-80, 10-70, 15-60, and/or 20-50 hours. In some embodiments, the circular RNA polynucleotide provided herein has a half-life greater than (e.g., at least 1.5-fold greater than, at least 2-fold greater than) that of an equivalent linear RNA polynucleotide encoding the same protein.

In some embodiments, the circular RNA provided herein may have a higher magnitude of expression than equivalent linear mRNA, e.g., a higher magnitude of expression 24 hours after administration of RNA to cells. In some embodiments, the circular RNA provided herein has a higher magnitude of expression than mRNA comprising the same expression sequence, 5moU modifications, an optimized UTR, a cap, and/or a polyA tail.

In some embodiments, the circular RNA provided herein may be less immunogenic than an equivalent mRNA when exposed to an immune system of an organism or a certain type of immune cell. In some embodiments, the circular RNA provided herein is associated with modulated production of cytokines when exposed to an immune system of an organism or a certain type of immune cell. For example, in some embodiments, the circular RNA provided herein is associated with reduced production of TNFα, RIG-I, IL-2, IL-6, IFNγ, and/or a type 1 interferon, e.g., IFN-β1, when exposed to an immune system of an organism or a certain type of immune cell as compared to mRNA comprising the same expression sequence. In some embodiments, the circular RNA provided herein is associated with less TNFα, RIG-I, IL-2, IL-6, IFNγ, and/or type 1 interferon, e.g., IFN-β1, transcript induction when exposed to an immune system of an organism or a certain type of immune cell as compared to mRNA comprising the same expression sequence. In some embodiments, the circular RNA provided herein is less immunogenic than mRNA comprising the same expression sequences. In some embodiments, the circular RNA provided herein is less immunogenic than mRNA comprising the same expression sequences, 5moU modifications, an optimized UTR, a cap, and/or a polyA tail.

In some embodiments, the compositions and methods described herein provide RNA (e.g., circRNA) with higher stability or functional stability than an equivalent linear RNA without the need for nucleoside modifications. In some embodiments, methods for producing RNA lacking nucleoside modifications produce higher percentages of full length transcripts than methods for producing RNA containing nucleoside modifications due to reduced abortive transcription. In some embodiments, the compositions and methods described herein are capable of producing large (e.g., 5 kb to 15 kb, 6 kb to 15 kb, 7 kb to 15 kb, 8 kb to 15 kb, 9 kb to 15 kb, 10 kb to 15 kb, 11 kb to 15 kb, 12 kb to 15 kb, 13 kb to 15 kb, 14 kb to 15 kb, 5 kb to 10 kb, 6 kb to 10 kb, 7 kb to 10 kb, 8 kb to 10 kb, 9 kb to 10 kb, 5 kb, 6 kb, 7 kb, 8 kb, 9 kb, 10 kb, 11, kb, 12 kb, 13 kb, 14 kb, or 15 kb) RNA constructs without the added abortive transcription associated with RNA containing nucleoside modifications.

In certain embodiments, the circular RNA provided herein can be transfected into a cell as is, or can be transfected in DNA vector form and transcribed in the cell. Transcription of circular RNA from a transfected DNA vector can be via added polymerases or polymerases encoded by nucleic acids transfected into the cell, or preferably via endogenous polymerases.

In certain embodiments, a circular RNA polynucleotide provided herein comprises modified RNA nucleotides and/or modified nucleosides. In some embodiments, the modified nucleoside is m⁵C (5-methylcytidine). In another embodiment, the modified nucleoside is m⁵U (5-methyluridine). In another embodiment, the modified nucleoside is m⁶A (N⁶-methyladenosine). In another embodiment, the modified nucleoside is s²U (2-thiouridine). In another embodiment, the modified nucleoside is Ψ (pseudouridine). In another embodiment, the modified nucleoside is Um (2′-O-methyluridine). In other embodiments, the modified nucleoside is m¹A (1-methyladenosine); m²A (2-methyladenosine); Am (2′-O-methyladenosine); ms²m⁶A (2-methylthio-N⁶-methyladenosine); i⁶A (N⁶-isopentenyladenosine); ms²i6A (2-methylthio-N⁶isopentenyladenosine); io⁶A (N⁶-(cis-hydroxyisopentenyl)adenosine); ms²io⁶A (2-methylthio-N⁶-(cis-hydroxyisopentenyl)adenosine); g⁶A (N⁶-glycinylcarbamoyladenosine); t⁶A (N⁶-threonylcarbamoyladenosine); ms²t⁶A (2-methylthio-N⁶-threonyl carbamoyladenosine); m⁶t⁶A (N⁶-methyl-N⁶-threonylcarbamoyladenosine); hn⁶A(N⁶-hydroxynorvalylcarbamoyladenosine); ms²hn⁶A (2-methylthio-N⁶-hydroxynorvalyl carbamoyladenosine); Ar(p) (2′-O-ribosyladenosine (phosphate)); I (inosine); m¹I (1-methylinosine); m¹Im (1,2′-O-dimethylinosine); m³C (3-methylcytidine); Cm (2′-O-methylcytidine); s²C (2-thiocytidine); ac⁴C (N⁴-acetylcytidine); f⁵C (5-formylcytidine); m¹Cm (5,2′-O-dimethylcytidine); ac⁴Cm (N⁴-acetyl-2′-O-methylcytidine); k²C (lysidine); m¹G (1-methylguanosine); m²G (N²-methylguanosine); m¹G (7-methylguanosine); Gm (2′-O-methylguanosine); m²₂G (N²,N²-dimethylguanosine); m²Gm (N²,2′-O-dimethylguanosine); m²₂Gm (N²,N²,2′-O-trimethylguanosine); Gr(p) (2′-O-ribosylguanosine(phosphate)); yW (wybutosine); o₂yW (peroxywybutosine); OHyW (hydroxywybutosine); OHyW* (undermodified hydroxywybutosine); imG (wyosine); mimG (methylwyosine); Q (queuosine); oQ (epoxyqueuosine); galQ (galactosyl-queuosine); manQ (mannosyl-queuosine); preQ₀ (7-cyano-7-deazaguanosine); preQ₁ (7-aminomethyl-7-deazaguanosine); G⁺ (archaeosine); D (dihydrouridine); m⁵Um (5,2′-O-dimethyluridine); s⁴U (4-thiouridine); m⁵s²U (5-methyl-2-thiouridine); s²Um (2-thio-2′-O-methyluridine); acp³U (3-(3-amino-3-carboxypropyl)uridine); ho⁵U (5-hydroxyuridine); mo⁵U (5-methoxyuridine); cmo⁵U (uridine 5-oxyacetic acid); mcmo⁵U (uridine 5-oxyacetic acid methyl ester); chm⁵U (5-(carboxyhydroxymethyl)uridine)); mchm⁵U (5-(carboxyhydroxymethyl)uridine methyl ester); mcm⁵U (5-methoxycarbonylmethyluridine); mcm⁵Um (5-methoxycarbonylmethyl-2′-O-methyluridine); mcm⁵s²U (5-methoxycarbonylmethyl-2-thiouridine); nm⁵S²U (5-aminomethyl-2-thiouridine); mnm⁵U (5-methylaminomethyluridine); mnm⁵s²U (5-methylaminomethyl-2-thiouridine); mnm⁵se²U (5-methylaminomethyl-2-selenouridine); ncm⁵U (5-carbamoylmethyluridine); ncm⁵Um (5-carbamoylmethyl-2′-O-methyluridine); cmnm⁵U (5-carboxymethylaminomethyluridine); cmnm⁵Um (5-carboxymethylaminomethyl-2′-O-methyluridine); cmnm³s²U (5-carboxymethylaminomethyl-2-thiouridine); m⁶₂A (N⁶,N⁶-dimethyladenosine); Im (2′-O-methylinosine); m⁴C (N⁴-methylcytidine); m⁴Cm (N⁴,2′-O-dimethylcytidine); hm⁵C(5-hydroxymethylcytidine); m³U (3-methyluridine); cm⁵U (5-carboxymethyluridine); m⁶Am (N⁶,2′-O-dimethyladenosine); m⁶₂Am (N⁶,N⁶,O-2′-trimethyladenosine); m^{2,7}G (N²,7-dimethylguanosine); m^{2,2,7}G (N²,N²,7-trimethylguanosine); m³Um (3,2′-O-dimethyluridine); m⁵D (5-methyldihydrouridine); f⁵Cm (5-formyl-2′-O-methylcytidine); m¹Gm (1,2′-O-dimethylguanosine); m¹Am (1,2′-O-dimethyladenosine); τm ⁵U (5-taurinomethyluridine); τms²U (5-taurinomethyl-2-thiouridine)); imG-14 (4-demethylwyosine); imG2 (isowyosine); or ac⁶A (N⁶-acetyladenosine).

In some embodiments, the modified nucleoside may include a compound selected from the group of: pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, 4-m ethoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine. In another embodiment, the modifications are independently selected from the group consisting of 5-methylcytosine, pseudouridine and 1-methylpseudouridine.

In some embodiments, the modified ribonucleosides include 5-methylcytidine, 5-methoxyuridine, 1-methyl-pseudouridine, N6-methyladenosine, and/or pseudouridine. In some embodiments, such modified nucleosides provide additional stability and resistance to immune activation.

In particular embodiments, polynucleotides may be codon-optimized. A codon optimized sequence may be one in which codons in a polynucleotide encoding a polypeptide have been substituted in order to increase the expression, stability and/or activity of the polypeptide. Factors that influence codon optimization include, but are not limited to one or more of: (i) variation of codon biases between two or more organisms or genes or synthetically constructed bias tables, (ii) variation in the degree of codon bias within an organism, gene, or set of genes, (iii) systematic variation of codons including context, (iv) variation of codons according to their decoding tRNAs, (v) variation of codons according to GC %, either overall or in one position of the triplet, (vi) variation in degree of similarity to a reference sequence for example a naturally occurring sequence, (vii) variation in the codon frequency cutoff, (viii) structural properties of mRNAs transcribed from the DNA sequence, (ix) prior knowledge about the function of the DNA sequences upon which design of the codon substitution set is to be based, and/or (x) systematic variation of codon sets for each amino acid. In some embodiments, a codon optimized polynucleotide may minimize ribozyme collisions and/or limit structural interference between the expression sequence and the IRES.

In certain embodiments circular RNA provided herein is produced inside a cell. In some embodiments, precursor RNA is transcribed using a DNA template (e.g., in some embodiments, using a vector provided herein) in the cytoplasm by a bacteriophage RNA polymerase, or in the nucleus by host RNA polymerase II and then circularized.

In certain embodiments, the circular RNA provided herein is injected into an animal (e.g., a human), such that a polypeptide encoded by the circular RNA molecule is expressed inside the animal.

### Payload

The circular RNA vaccines of the invention comprise one or more circular RNA polynucleotides, which encode one or more wild type or engineered proteins, peptides or polypeptides (e.g., antigens, adjuvant, or adjuvant-like proteins). In some embodiments, the one or more circular RNA polynucleotide encodes an antigen or adjuvant derived from an infectious agent. In some embodiments the infectious agent from which the antigen or adjuvant is derived or engineered includes, but is not limited to a virus, bacterium, fungus, protozoan, and/or parasite. In some embodiments, the antigen is a viral antigen. In an embodiment, the antigen is a SARS-CoV-2 antigen. In an embodiment, the antigen is SARS-CoV-2 spike protein.

In some embodiments, a circular RNA polynucleotide comprises more than one expression sequence. In some embodiments, an expression sequence may encode more than one antigenic polypeptide. In some embodiments, the expression sequence of the one or more RNA polynucleotides encodes at least 2, 3, 4, 5, 6, 7, 8, 9 or 10 antigenic polypeptides. In some embodiments, the expression sequence of the one or more RNA polynucleotides encodes at least 10, 15, 20 or 50 antigenic polypeptides. In some embodiments, the expression sequence of the one or more RNA polynucleotides encodes 2-10, 10-15, 15-20, 20-50, 50-100 or 100-200 antigenic polypeptides.

In an embodiment, the antigen is selected from or derived from the group consisting of rotavirus, foot and mouth disease virus, influenza A virus, influenza B virus, influenza C virus, H1N1, H2N2, H3N2, H5N1, H7N7, H1N2, H9N2, H7N2, H7N3, H10N7, human parainfluenza type 2, herpes simplex virus, Epstein-Barr virus, varicella virus, porcine herpesvirus 1, cytomegalovirus, lyssavirus, Bacillus anthracis, anthrax PA and derivatives, poliovirus, Hepatitis A, Hepatitis B, Hepatitis C, Hepatitis E, distemper virus, venezuelan equine encephalomyelitis, feline leukemia virus, reovirus, respiratory syncytial virus, Lassa fever virus, polyoma tumor virus, canine parvovirus, papilloma virus, tick borne encephalitis virus, rinderpest virus, human rhinovirus species, Enterovirus species, Mengovirus, paramyxovirus, avian infectious bronchitis virus, human T-cell leukemia-lymphoma virus 1, human immunodeficiency virus-1, human immunodeficiency virus-2, lymphocytic choriomeningitis virus, parvovirus B19, adenovirus, rubella virus, yellow fever virus, dengue virus, bovine respiratory syncitial virus, corona virus, Bordetella pertussis, Bordetella bronchiseptica, Bordetella parapertussis, Brucella abortis, Brucella melitensis, Brucella suis, Brucella ovis, Brucella species, Escherichia coli, Salmonella species, Salmonella typhi, Streptococci, Vibrio cholera, Vibrio parahaemolyticus, Shigella, Pseudomonas, tuberculosis, avium, Bacille Calmette Guerin, Mycobacterium leprae, Pneumococci, Staphlylococci, Enterobacter species, Rochalimaia henselae, Pasteurella haemolytica, Pasteurella multocida, Chlamydia trachomatis, Chlamydia psittaci, Lymphogranuloma venereum, Treponema pallidum, Haemophilus species, Mycoplasma bovigenitalium, Mycoplasma pulmonis, Mycoplasma species, Borrelia burgdorferi, Legionalla pneumophila, Colstridium botulinum, Corynebacterium diphtheriae, Yersinia entercolitica, Rickettsia rickettsii, Rickettsia typhi, Rickettsia prowsaekii, Ehrlichia chaffeensis, Anaplasma phagocytophilum, Plasmodium falciparum, Plasmodium vivax, Plasmodium malariae, Schistosomes, trypanosomes, Leishmania species, Filarial nematodes, trichomoniasis, sarcosporidiasis, Taenia saginata, Taenia solium, Leishmania, Toxoplasma gondii, Trichinella spiralis, coccidiosis, Eimeria tenella, Cryptococcus neoformans, Candida albican, Aspergillus fumigatus, coccidioidomycosis, Neisseria gonorrhoeae, malaria circumsporozoite protein, malaria merozoite protein, trypanosome surface antigen protein, pertussis, alphaviruses, adenovirus, diphtheria toxoid, tetanus toxoid, meningococcal outer membrane protein, streptococcal M protein, Influenza hemagglutinin, cancer antigen, tumor antigens, toxins, Clostridium perfringens epsilon toxin, ricin toxin, pseudomonas exotoxin, exotoxins, neurotoxins, cytokines, cytokine receptors, monokines, monokine receptors, plant pollens, animal dander, and dust mites.

In some embodiments, the adjuvant is selected from or derived from the group consisting of BCSP31, MOMP, FomA, MymA, ESAT6, PorB, PVL, Porin, OmpA, PepO, OmpU, Lumazine synthase, Omp16, Omp19, CobT, RpfE, Rv0652, HBHA, NhhA, DnaJ, Pneumolysin, Falgellin, IFN-alpha, IFN-gamma, IL-2, IL-12, IL-15, IL-18, IL-21, GM-CSF, IL-1b, IL-6, TNF-α, IL-7, IL-17, IL-1Beta, anti-CTLA4, anti-PD1, anti-41BB, PD-L1, Tim-3, Lag-3, TIGIT, GITR, and andti-CD3.

### Immunogenic Vectors & RNA Preparations

In some embodiments, the circular RNA vaccine of the invention comprises one or more circular RNA polynucleotide or linear RNA polynucleotide counterpart capable of triggering an immune response in a cell. Modifications or engineering of non-immunogenic circular RNA polynucleotide can allow for adjuvant-like properties (Wesselhoeft, 2019). Similarly, linear RNA polynucleotides can be engineered to trigger an increased immune response than a non-engineered linear RNA polynucleotide. Examples of the increased immunogenicity for linear RNA polynucleotides include various capping strategies (Pardi, 2018). Capping strategies include, but are not limited to, incorporation of a monophosphorylated or a triphosphorylated at the terminal 5′ end by adding a nucleotide monosphosphate to the in vivo transcription reaction. In some embodiments, varying the ratios of triphosphorylated; monophosphorylated 5′ terminal caps in an RNA preparation may be controlled based on altering the GMP:GTP ratio during an in vivo transcription. In other embodiments, an enzyme (e.g., RppH) may be used to control the ratio of triphosphorylated:monophosphorylated 5′ terminal caps in an RNA preparation. The ratio of monosphorylated:trisphosphorylated in any RNA preparation may be a 100:1 90:10, 80:20, 70:30, 60:40, 50:50, 40:60, 30:70, 20:80, 10:90, 100:1 based on preferred levels of immunogenicity. Greater ratios of trisphosphorylation:monosphorylation ratios allows for greater immune response activation.

In some embodiments, a monophosphate or triphosphate inclusion cap may be produced using the synthesis method based providing initiator molecules during the development of the RNA polynucleotide. In some embodiments, the number of triphosphates at the 5′ end of an RNA molecule produced by in vitro transcription can be controlled by including specific nucleotides and/or nucleosides in the in vitro transcription reaction. These nucleotides which will then be used with varying efficiency as initiator nucleotides/nucleosides for new RNA strands. In the same embodiment, an RNA polymerase enzyme (e.g., T7 RNA polymerase) has the ability to stochastically choose an initiator nucleotide/nucleoside from available substrates. In some embodiments, including multiple different initiator nucleosides/nucleotides (e.g., GTP and GMP) into the synthesis will result in some RNA molecules with 5′ monophosphates and some with 5′ triphosphates. The ratio of initiator nucleotides/nucleosides used and the rate of incorporation for a specific nucleotide/nucleoside will determine the proportion of RNA molecules with a specific 5′ terminal identity. In a preferred embodiment for generating RNA molecules with monophosphate 5′ termini, GMP is added to T7 RNA polymerase in vitro transcription reactions at greater than or equal to 1× the starting concentration of GTP, most preferably 4×. In some embodiments, an alternative initiator molecule may be used such as an adenosine nucleotide/nucleoside, particularly when using an alternative RNA polymerase enzyme.

In another embodiment, a method of monophosphate or triphosphate inclusion cap may include the splicing method. A guanosine nucleotide/nucleoside may be incorporated before the second splice site dinucleotide of the 5′ splice site during group I intron and permuted group I intron splicing. This nucleotide/nucleoside can include zero or more phosphate groups at the 5′ position. Including multiple different nucleosides/nucleotides (e.g. GTP and GMP) will result in some intron products with 5′ monophosphates and some with 5′ triphosphates. The ratio of nucleotides/nucleosides used and the rate of utilization for a specific nucleotide/nucleoside by the group I intron will determine the proportion of RNA molecules with a specific 5′ terminal identity. In a preferred embodiment, the ratio of nucleosides/nucleotides used is identical to that used for in vitro transcription of precursor molecules and splicing occurs co-transcriptionally. The ratio can be independently controlled by purifying precursor RNA molecules from an in vitro transcription reaction and adding necessary cofactors for splicing along with the desired ratio of nucleosides/nucleotides. Group I introns generally only accept guanosine nucleotides/nucleosides as cofactors but may sometimes accept other nucleotides/nucleosides such as adenosine nucleotides/nucleosides.

In another embodiment, a monophosphate or triphosphate inclusion cap may be produced using an enzymatic method. Triphosphate termini can be converted to monophosphate or hydroxyl termini through enzymatic treatment. Treatment of triphosphorylated RNA molecules with RNA 5′ Pyrophosphohydrolase (RppH) or Tobacco acid pyrophosphatase (TAP) converts a triphosphorylated terminus into a monophosphorylated terminus, which can then be used for ligation by ligase enzymes such as T4 RNA Ligase I, and will not trigger RIG-I. Other phosphatase enzymes such as Calf Intestinal Phosphatase (CIP/CIAP), Shrimp Alkaline Phosphatase (SAP), and others remove terminal phosphates, thereby converting a terminal monophosphate, diphosphate, or triphosphate into a terminal hydroxyl group. Terminal hydroxyl groups can then be converted into monophosphate groups using a kinase enzyme such as T4 Polynucleotide Kinase (PNK).

In some embodiments, RNA preparations can be made more immune stimulatory by using different structures or formulations of RNA polynucleotides in varying percentages. In other embodiments, RNA preparations may contain both non-immunostimulatory circular RNA polynucleotides and linear RNA polynucleotides containing 5′ termini caps or immunostimulatory-modified circular RNA polynucleotides. In certain embodiments, the RNA preparations contain circular RNA polynucleotides encoding an adjuvant, antigen or adjuvant-like protein along with linear RNA polynucleotides or immunostimulatory-modified circular RNA to help stimulate an immune response.

### Additional Targets and Combinations

In some embodiments, provided are methods for treating or preventing a microbial infection (e.g., a bacterial or viral infection) and/or a disease, disorder, or condition associated with a microbial or viral infection, or a symptom thereof, in a subject, by administering a circular RNA vaccine comprising one or more polynucleotides encoding one or more peptides. The administration may be in combination with an antimicrobial agent, e.g., an anti-bacterial agent., an anti-microbial polypeptide, or a small molecule anti-microbial compound described herein. Anti-microbial agents can include, but are not limited to, anti-bacterial agents, anti-viral agents, anti-fungal agents, anti-protozoal agents, anti-parasitic agents, and anti-prion agents.

Conditions Associated with Bacterial Infection

Diseases, disorders, or conditions which may be associated with bacterial infections which may be treated using the circular RNA vaccine of the invention include, but are not limited to one or more of the following: abscesses, actinomycosis, acute prostatitis, Aeromonas hydrophila, annual ryegrass toxicity, anthrax, bacillary peliosis, bacteremia, bacterial gastroenteritis, bacterial meningitis, bacterial pneumonia, bacterial vaginosis, bacterium-related cutaneous conditions, bartonellosis, BCG-oma, botryomycosis, botulism, Brazilian purpuric fever, Brodie abscess, brucellosis, Buruli ulcer, campylobacteriosis, caries, Carrion's disease, cat scratch disease, cellulitis, chlamydia infection, cholera, chronic bacterial prostatitis, chronic recurrent multifocal osteomyelitis, clostridial necrotizing enteritis, combined periodontic-endodontic lesions, contagious bovine pleuropneumonia, diphtheria, diphtheritic stomatitis, ehrlichiosis, erysipelas, piglottitis, erysipelas, Fitz-Hugh-Curtis syndrome, flea-borne spotted fever, foot rot (infectious pododermatitis), Garre's sclerosing osteomyelitis, Gonorrhea, Granuloma inguinale, human granulocytic anaplasmosis, human monocytotropic ehrlichiosis, hundred days' cough, impetigo, late congenital syphilitic oculopathy, legionellosis, Lemierre's syndrome, leprosy (Hansen's Disease), leptospirosis, listeriosis, Lyme disease, lymphadenitis, melioidosis, meningococcal disease, meningococcal septicaemia, methicillin-resistant Staphylococcus aureus (MRS A) infection, Mycobacterium avium-intracellulare (MAI), mycoplasma pneumonia, necrotizing fasciitis, nocardiosis, noma (cancrum oris or gangrenous stomatitis), omphalitis, orbital cellulitis, osteomyelitis, overwhelming post-splenectomy infection (OPSI), ovine brucellosis, pasteurellosis, periorbital cellulitis, pertussis (whooping cough), plague, pneumococcal pneumonia, Pott disease, proctitis, pseudomonas infection, psittacosis, pyaemia, pyomyositis, Q fever, relapsing fever (typhinia), rheumatic fever, Rocky Mountain spotted fever (RMSF), rickettsiosis, salmonellosis, scarlet fever, sepsis, serratia infection, shigellosis, southern tick-associated rash illness, staphylococcal scalded skin syndrome, streptococcal pharyngitis, swimming pool granuloma, swine brucellosis, syphilis, syphilitic aortitis, tetanus, toxic shock syndrome (TSS), trachoma, trench fever, tropical ulcer, tuberculosis, tularemia, typhoid fever, typhus, urogenital tuberculosis, urinary tract infections, vancomycin-resistant Staphylococcus aureus infection, Waterhouse-Friderichsen syndrome, pseudotuberculosis (Yersinia) disease, and yersiniosis

### Bacterial Pathogens

The bacterium described herein can be a Gram-positive bacterium or a Gram-negative bacterium. Bacterial pathogens include, but are not limited to, Acinetobacter baumannii, Bacillus anthracis, Bacillus subtilis, Bordetella pertussis, Borrelia burgdorferi, Brucella abortus, Brucella canis, Brucella melitensis, Brucella suis, Campylobacter jejuni, Chlamydia pneumoniae, Chlamydia trachomatis, Chlamydophila psittaci, Clostridium botulinum, Clostridium difficile, Clostridium perfringens, Clostridium tetani, coagulase Negative Staphylococcus, Corynebacterium diphtheria, Enterococcus faecalis, Enterococcus faecium, Escherichia coli, enterotoxigenic Escherichia coli (ETEC), enteropathogenic E. coli, E. coli 0157:H7, Enter obacter sp., Francisella tularensis, Haemophilus influenzae, Helicobacter pylori, Klebsiella pneumoniae, Legionella pneumophila, Leptospira interrogans, Listeria monocytogenes, Moraxella catarralis, Mycobacterium leprae, Mycobacterium tuberculosis, Mycoplasma pneumoniae, Neisseria gonorrhoeae, Neisseria meningitides, Preteus mirabilis, Proteus sps., Pseudomonas aeruginosa, Rickettsia rickettsii, Salmonella typhi, Salmonella typhimurium, Serratia marcescens, Shigella flexneri, Shigella sonnei, Staphylococcus aureus, Staphylococcus epidermidis, Staphylococcus saprophyticus, Streptococcus agalactiae, Streptococcus mutans, Streptococcus pneumoniae, Streptococcus pyogenes, Treponema pallidum, Vibrio cholerae, and Yersinia pestis.

Bacterial pathogens may also include bacteria that cause resistant bacterial infections, for example, clindamycin-resistant Clostridium difficile, fluoroquinolone—resistant Clostridium difficile, methicillin-resistant Staphylococcus aureus (MRS A), multidrug—resistant Enterococcus faecalis, multidrug-resistant Enterococcus faecium, multidrug-resistance Pseudomonas aeruginosa, multidrug-resistant Acinetobacter baumannii, and vancomycin-resistant Staphylococcus aureus (VRSA).

### Antibiotic Combinations

In some embodiments, the circular RNA vaccine of the present invention, e.g., circular RNA vaccine comprising one or more antigen-encoding polynucleotides of the present invention, may be administered in conjunction with one or more antibacterial agent.

### Antibacterial Agents

Antibacterial agents include, but are not limited to, aminoglycosides (e.g., amikacin (AMIKIN®), gentamicin (GARAMYCN®), kanamycin (KANTREX®), neomycin (MYCIFRADIN®), netilmicin (NETROMYCIN®), tobramycin (NEBCIN®), Paromomycin (HUMATIN®)), ansamycins (e.g., geldanamycin, herbimycin), carbacephem (e.g., loracarbef (LORABID®), Carbapenems (e.g., ertapenem (INVANZ®), doripenem (DORIBAX®), imipenem/cilastatin (PRIMAXIN®), meropenem (MERREM®), cephalosporins (first generation) (e.g., cefadroxil (DURICEF®), cefazolin (ANCEF®), cefalotin or cefalothin (KEFLIN®), cefalexin (KEFLEX®), cephalosporins (second generation) (e.g., cefaclor (CECLOR®), cefamandole (MANDOL®), cefoxitin (MEFOXIN®), cefprozil (CEFZIL®), cefuroxime (CEFTIN®, ZINNAT®)), cephalosporins (third generation) (e.g., cefixime (SUPRAX®), cefdinir (OMNICEF®, CEFDIEL®), cefditoren (SPECTRACEF®), cefoperazone (CEFOBID®), cefotaxime (CLAFORAN®), cefpodoxime (VANTIN®), ceftazidime (FORTAZ®), ceftibuten (CEDAX®), ceftizoxime (CEFIZOX®), ceftriaxone (ROCEPHIN®)), cephalosporins (fourth generation) (e.g., cefepime (MAXIPIME®)), cephalosporins (fifth generation) (e.g., ceftobiprole (ZEFTERA®)), glycopeptides (e.g., teicoplanin (TARGOCID®), vancomycin (VANCOCIN®), telavancin (VIBATIV®)), lincosamides (e.g., clindamycin (CLEOCIN®), lincomycin (LINCOCIN®)), lipopeptide (e.g., daptomycin (CUBICIN®)), macrolides (e.g., azithromycin (ZITHROMAX®, SUMAMED®, ZITROCIN®), clarithromycin (BIAXIN®), dirithromycin (DYNABAC®), erythromycin (ERYTHOCIN®, ERYTHROPED®), roxithromycin, troleandomycin (TAO®), telithromycin (KETEK®), spectinomycin (TROBICIN®)), monobactams (e.g., aztreonam (AZACTAM®)), nitrofurans (e.g., furazolidone (FUROXONE®), nitrofurantoin (MACRODANTIN®, MACROBID®)), penicillins (e.g., amoxicillin (NOVAMOX®, AMOXIL®), ampicillin (PRINCIPEN®), azlocillin, carbenicillin (GEOCILLIN®), cloxacillin (TEGOPEN®), dicloxacillin (DYNAPEN®), flucloxacillin (FLOXAPEN®), mezlocillin (MEZLIN®), methicillin (STAPHCILLIN®), nafcillin (UNIPEN®), oxacillin (PROSTAPHLIN®), penicillin G (PENTIDS®), penicillin V (PEN-VEE-K®), piperacillin (PIPRACIL®), temocillin (NEGABAN®), ticarcillin (TICAR®)), penicillin combinations (e.g., amoxicillin/clavulanate (AUGMENTIN®), ampicillin/sulbactam (UNASYN®), piperacillin/tazobactam (ZOSYN®), ticarcillin/clavulanate (TIMENTIN®)), polypeptides (e.g., bacitracin, colistin (COLY-MYCIN-S®), polymyxin B, quinolones (e.g., ciprofloxacin (CIPRO®, CIPROXIN®, CIPROBAY®), enoxacin (PENETREX®), gatifloxacin (TEQUIN®), levofloxacin (LEVAQUIN®), lomefloxacin (MAXAQUIN®), moxifloxacin (AVELOX®), nalidixic acid (NEGGRAM®), norfloxacin (NOROXIN®), ofloxacin (FLOXIN®, OCUFLOX®), trovafloxacin (TROVAN®), grepafloxacin (RAXAR®), sparfloxacin (ZAGAM®), temafloxacin (OMNIFLOX®)), sulfonamides (e.g., mafenide (SULFAMYLON®), sulfonamidochrysoidine (PRONTOSIL®), sulfacetamide (SULAMYD®, BLEPH-10®), sulfadiazine (MICRO-SULFON®), silver sulfadiazine (SILVADENE®), sulfamethizole (THIOSULFIL FORTE®), sulfamethoxazole (GANTANOL®), sulfanilimide, sulfasalazine (AZULFIDINE®), sulfisoxazole (GANTRISIN®), trimethoprim (PROLOPREVI®), TRIMPEX®), trimethoprim-sulfamethoxazole (co-trimoxazole) (TMP-SMX) (BACTRIM®, SEPTRA®)), tetracyclines (e.g., demeclocycline (DECLOMYCIN®), doxycycline (VIBRAMYCIN®), minocycline (MINOCIN®), oxytetracycline (TERRAMYCIN®), tetracycline (SUMYCIN®, ACHROMYCIN® V, STECLIN®)), drugs against mycobacteria (e.g., clofazimine (LAMPRENE®), dapsone (AVLOSULFON®), capreomycin (CAPASTAT®), cycloserine (SEROMYCIN®), ethambutol (MYAMBUTOL®), ethionamide (TRECATOR®), isoniazid (I.N.H.®), pyrazinamide (ALDIN AMIDE®), rifampin (RIFADIN®, RIMACTANE®), rifabutin (MYCOBUTIN®), rifapentine (PRIFTIN®), streptomycin), and others (e.g., arsphenamine (SALVARSAN®), chloramphenicol (CHLOROMYCETIN®), fosfomycin (MONUROL®), fusidic acid (FUCIDIN®), linezolid (ZYVOX®), metronidazole (FLAGYL®), mupirocin (BACTROBAN®), platensimycin, quinupristin/dalfopristin (SYNERCID®), rifaximin (XIFAXAN®), thiamphenicol, tigecycline (TIGACYL®), tinidazole (TINDAMAX®, FASIGYN®)).

Conditions Associated with Viral Infection

In some embodiments, provided are methods for treating or preventing a viral infection and/or a disease, disorder, or condition associated with a viral infection, or a symptom thereof, in a subject, by administering a circular RNA vaccine comprising one or more polynucleotides encoding an anti-viral polypeptide, e.g., an anti-viral polypeptide described herein. In some embodiments, the circular RNA vaccine is administered in combination with an anti-viral agent, e.g., an anti-viral polypeptide or a small molecule anti-viral agent described herein.

Diseases, disorders, or conditions associated with viral infections which may be treated using the circular RNA vaccines of the invention include, but are not limited to, acute febrile pharyngitis, pharyngoconjunctival fever, epidemic keratoconjunctivitis, infantile gastroenteritis, Coxsackie infections, infectious mononucleosis, Burkitt lymphoma, acute hepatitis, chronic hepatitis, hepatic cirrhosis, hepatocellular carcinoma, primary HSV-1 infection (e.g., gingivostomatitis in children, tonsillitis and pharyngitis in adults, keratoconjunctivitis), latent HSV-1 infection (e.g., herpes labialis and cold sores), primary HSV-2 infection, latent HSV-2 infection, aseptic meningitis, infectious mononucleosis, Cytomegalic inclusion disease, Kaposi sarcoma, multicentric Castleman disease, primary effusion lymphoma, AIDS, influenza, Reye syndrome, measles, postinfectious encephalomyelitis, Mumps, hyperplastic epithelial lesions (e.g., common, flat, plantar and anogenital warts, laryngeal papillomas, epidermodysplasia verruciformis), cervical carcinoma, squamous cell carcinomas, croup, pneumonia, bronchiolitis, common cold, Poliomyelitis, Rabies, bronchiolitis, pneumonia, influenza-like syndrome, severe bronchiolitis with pneumonia, German measles, congenital rubella, Varicella, herpes zoster, and SARS-CoV-2.

### Viral Pathogens

Examples of viral infectious agents include, but are not limited to, adenovirus; Herpes simplex, type 1; Herpes simplex, type 2; encephalitis virus; papillomavirus; Varicella-zoster virus; Epstein-barr virus; Human cytomegalovirus; Human herpesvirus, type 8; Human papillomavirus; BK virus; JC virus; Smallpox; polio virus, Hepatitis B virus; Human bocavirus; Parvovirus B19; Human astrovirus; Norwalk virus; coxsackievirus; hepatitis A virus; poliovirus; rhinovirus; Severe acute respiratory syndrome virus; Hepatitis C virus; yellow fever virus; dengue virus; West Nile virus; Rubella virus; Hepatitis E virus, Human immunodeficiency virus (HIV); Influenza virus, type A or B; Guanarito virus; Junin virus, Lassa virus; Machupo virus; Sabia virus; Crimean-Congo hemorrhagic fever virus; Ebola virus; Marburg virus; Measles virus; Mumps virus; Parainfluenza virus; Respiratory syncytial virus; Human metapneumovirus; Hendra virus; Nipah virus; Rabies virus; Hepatitis D; Rotavirus; Orbivirus; Coltivirus; Hantavirus, Middle East Respiratory Coronavirus; Chikungunya virus, Banna virus, or SARS-CoV-2. Viral pathogens may also include viruses that cause anti-viral resistant infections.

### Antiviral Agents

Exemplary anti-viral agents include, but are not limited to, abacavir (ZIAGEN®), abacavir/lamivudine/zidovudine (Trizivir®), aciclovir or acyclovir (CYCLOVIR®, HERPEX®, ACIVIR®, ACIVIRAX®, ZOVIRAX®, ZOVIR®), adefovir (Preveon®, Hepsera®), amantadine (SYMMETREL®), amprenavir (AGENERASE®), ampligen, arbidol, atazanavir (REYATAZ®), boceprevir, cidofovir, darunavir (PREZISTA®), delavirdine (RESCRIPTOR®), didanosine (VIDEX®), docosanol (ABREVA®), edoxudine, efavirenz (SUSTINA®, STOCRIN®), emtricitabine (EMTRIVA®), emtricitabine/tenofovir/efavirenz (ATRIPLA®), enfuvirtide (FUZEON®), entecavir (BARACLUDE®, ENNAVIR®), famciclovir (FAMVIR®), fomivirsen (VITRA VENE®), fosamprenavir (LEXIVA®, TELZIR®), foscarnet (FOSCAVIR®), fosfonet, ganciclovir (CYTOVENE®, CYMEVENE®, VITRASERT®), GS 9137 (ELVITEGRAVIR®), imiquimod (ALDARA®, ZYCLARA®, BESELNA®), indinavir (CRIXIVAN®), inosine, inosine pranobex (IMUNOVIR®), interferon type I, interferon type II, interferon type III, kutapressin (NEXAVIR®), lamivudine (ZEFFIX®, HEPTOVIR®, EPIVIR®), lamivudine/zidovudine (COMBIVIR®), lopinavir, loviride, maraviroc (SELZENTRY®, CELSENTRI®), methisazone, MK-2048, moroxydine, nelfinavir (VIRACEPT®), nevirapine (VIRAMUNE®), oseltamivir (TAMIFLU®), peginterferon alfa-2a (PEGASYS®), penciclovir (DENAVIR®), peramivir, pleconaril, podophyllotoxin (CONDYLOX®), raltegravir (ISENTRESS®), ribavirin (COPEGUs®, REBETOL®, RIBASPHERE®, VILONA® AND VIRAZOLE®), rimantadine (FLUMADINE®), ritonavir (NORVIR®), pyramidine, saquinavir (INVIRASE®, FORTOVASE®), stavudine, tea tree oil (melaleuca oil), tenofovir (VIREAD®), tenofovir/emtricitabine (TRUVADA®), tipranavir (APTIVUS®), trifluridine (VIROPTIC®), tromantadine (VIRU-MERZ®), valaciclovir (VALTREX®), valganciclovir (VALCYTE®), vicriviroc, vidarabine, viramidine, zalcitabine, zanamivir (RELENZA®), and zidovudine (azidothymidine (AZT), RETROVIR®, RETROVIS®).

Conditions Associated with Fungal Infections

Diseases, disorders, or conditions associated with fungal infections which may be treated using the circular RNA vaccines of the invention include, but are not limited to, aspergilloses, blastomycosis, candidasis, coccidioidomycosis, cryptococcosis, histoplasmosis, mycetomas, paracoccidioidomycosis, and tinea pedis. Furthermore, persons with immuno-deficiencies are particularly susceptible to disease by fungal genera such as Aspergillus, Candida, Cryptoccocus, Histoplasma, and Pneumocystis, which can be treated using the circular RNA vaccines of the invention. Other fungi that can be treated using the circular RNA vaccines of the invention include fungi that can attack eyes, nails, hair, and especially skin, the so-called dermatophytic fungi and keratinophilic fungi, which cause a variety of conditions, of which ringworms such as athlete's foot are common. Circular RNA vaccines of the present invention can also be used to treat allergies caused by fungal spores, and fungi from a variety of taxonomic groups.

### Fungal Pathogens

Fungal pathogens include, but are not limited to, Ascomycota (e.g., Fusarium oxysporum, Pneumocystis jirovecii, Aspergillus spp., Coccidioides immitis/posadasii, Candida albicans), Basidiomycota (e.g., Filobasidiella neoformans, Trichosporon), Microsporidia (e.g., Encephalitozoon cuniculi, Enterocytozoon bieneusi), and Mucoromycotina (e.g., Mucor circinelloides, Rhizopus oryzae, Lichtheimia corymbifera).

### Anti-Fungal Agents

Anti-fungal agents that can be used in combination with the circular RNA vaccines of the present invention include, but are not limited to, polyene antifungals (e.g., natamycin, rimocidin, filipin, nystatin, amphotericin B, candicin, hamycin), imidazole antifungals (e.g., miconazole (MICATIN®, DAKTARIN®), ketoconazole (NIZORAL®, FUNGORAL®, SEBIZOLE®), clotrimazole (LOTRIMIN®, LOTRIMIN® AF, CANESTEN®), econazole, omoconazole, bifonazole, butoconazole, fenticonazole, isoconazole, oxiconazole, sertaconazole (ERTACZO®), sulconazole, tioconazole), triazole antifungals (e.g., albaconazole fluconazole, itraconazole, isavuconazole, ravuconazole, posaconazole, voriconazole, terconazole), thiazole antifungals (e.g., abafungin), allylamines (e.g., terbinafine (LAMISIL®), naftifine (NAFTIN®), butenafine (LOTRIMIN® Ultra)), echinocandins (e.g., anidulafungin, caspofungin, micafungin), and others (e.g., polygodial, benzoic acid, ciclopirox, tolnaftate (TINACTIN®, DESENEX®, AFTATE®), undecylenic acid, flucytosine or 5-fluorocytosine, griseofulvin, haloprogin, sodium bicarbonate, allicin).

Conditions Associated with Protozoal Infection

Diseases, disorders, or conditions associated with protozoal infections which may be treated using the circular RNA vaccines of the invention include, but are not limited to, amoebiasis, giardiasis, trichomoniasis, African Sleeping Sickness, American Sleeping Sickness, leishmaniasis (Kala-Azar), balantidiasis, toxoplasmosis, malaria, Acanthamoeba keratitis, and babesiosis.

### Protozoan Pathogens

Protozoal pathogens include, but are not limited to, Entamoeba histolytica, Giardia lambila, Trichomonas vaginalis, Trypanosoma brucei, T. cruzi, Leishmania donovani, Balantidium coli, Toxoplasma gondii, Plasmodium spp., and Babesia microti.

### Anti-Protozoan Agents

Exemplary anti-protozoal agents include, but are not limited to, eflornithine, furazolidone (FUROXONE®, DEPEND AL-M®), melarsoprol, metronidazole (FLAGYL®), ornidazole, paromomycin sulfate (HUMATIN®), pentamidine, pyrimethamine (DARAPRIM®), and tinidazole (TINDAMAX®, FASIGYN®).

Conditions Associated with Parasitic Infection

Diseases, disorders, or conditions associated with parasitic infections which may be treated using the circular RNA vaccines of the invention include, but are not limited to, Acanthamoeba keratitis, amoebiasis, ascariasis, babesiosis, balantidiasis, baylisascariasis, chagas disease, clonorchiasis, cochliomyia, cryptosporidiosis, diphyllobothriasis, dracunculiasis, echinococcosis, elephantiasis, enterobiasis, fascioliasis, fasciolopsiasis, filariasis, giardiasis, gnathostomiasis, hymenolepiasis, isosporiasis, katayama fever, leishmaniasis, lyme disease, malaria, metagonimiasis, myiasis, onchocerciasis, pediculosis, scabies, schistosomiasis, sleeping sickness, strongyloidiasis, taeniasis, toxocariasis, toxoplasmosis, trichinosis, and trichuriasis.

### Parasitic Pathogens

Parasitic pathogens include, but are not limited to, Acanthamoeba, Anisakis, Ascaris lumbricoides, botfly, Balantidium coli, bedbug, Cestoda, chiggers, Cochliomyia hominivorax, Entamoeba histolytica, Fasciola hepatica, Giardia lamblia, hookworm, Leishmania, Linguatula serrata, liver fluke, Loa loa, Paragonimus, pinworm, Plasmodium falciparum, Schistosoma, Strongyloides stercoralis, mite, tapeworm, Toxoplasma gondii, Trypanosoma, whipworm, and Wuchereria bancrofti.

### Anti-Parasitic Agents

Exemplary anti-parasitic agents include, but are not limited to, antinematodes (e.g., mebendazole, pyrantel pamoate, thiabendazole, diethylcarbamazine, ivermectin), anticestodes (e.g., niclosamide, praziquantel, albendazole), antitrematodes (e.g., praziquantel), antiamoebics (e.g., rifampin, amphotericin B), and antiprotozoals (e.g., melarsoprol, eflornithine, metronidazole, tinidazole).

### Cleavage Site

In some embodiments, two or more expression sequences in a polynucleotide construct may be separated by one or more cleavage site sequences. A cleavage site may be any sequence which enables the two or more polypeptides to become separated. A cleavage site may be self-cleaving, such that when the polypeptide is produced, it is immediately cleaved into individual polypeptides without the need for any external cleavage activity.

In some embodiments, a cleavage site may be a furin cleavage site. Furin is an enzyme which belongs to the subtilisin-like proprotein convertase family. The members of this family are proprotein convertases that process latent precursor proteins into their biologically active products. Furin is a calcium-dependent serine endoprotease that can efficiently cleave precursor proteins at their paired basic amino acid processing sites. Examples of furin substrates include proparathyroid hormone, transforming growth factor beta 1 precursor, proalbumin, pro-beta-secretase, membrane type-1 matrix metalloproteinase, beta subunit of pro-nerve growth factor and von Willebrand factor. Furin cleaves proteins just downstream of a basic amino acid target sequence (canonically, Arg-X-(Arg/Lys)-Arg) and is enriched in the Golgi apparatus.

In some embodiments, a cleavage site may encode a self-cleaving peptide.

In some embodiments, a cleavage site may operate by ribosome skipping such as the skipping of a glycyl-propyl bond at the C-terminus of a 2A self-cleaving peptide. In some embodiments, steric hinderance causes ribosome skipping. In some embodiments, a 2A self-cleaving peptide contains the sequence GDVEXNPGP (SEQ ID NO. 324), wherein X is E or S. In some embodiments, the protein encoded upstream of the 2A self-cleaving peptide is attached to the 2A self-cleaving peptide except the C-terminal proline post translation. In some embodiments, the protein encoded downstream of the 2A self-cleaving peptide is attached to a proline at its N-terminus post translation.

In some embodiments, a self-cleaving peptide may be a 2A self-cleaving peptide from an aphtho- or a cardiovirus. The primary 2A/2B cleavage of the aptho- and cardioviruses is mediated by 2A cleaving at its own C-terminus. In apthoviruses, such as foot-and-mouth disease viruses (FMDV) and equine rhinitis A virus, the 2A region is a short section of about 18 amino acids, which, together with the N-terminal residue of protein 2B (a conserved proline residue) represents an autonomous element capable of mediating cleavage at its own C-terminus (Donelly et al. (2001)).

2A-like sequences have been found in picornaviruses other than aptho- or cardioviruses, ‘picornavirus-like’ insect viruses, type C rotaviruses and repeated sequences within Trypanosoma spp and a bacterial sequence (Donnelly et al. (2001)). In some embodiments, the cleavage site may comprise one of these 2A-like sequences, such as those listed in Table 8.

In some embodiments, a self-cleaving peptide is F2A. In some embodiments, a self-cleaving peptide is derived from foot-and-mouth disease virus. In some embodiments, a self-cleaving peptide is E2A. In some embodiments, a self-cleaving peptide is derived from equine rhinitis A virus. In some embodiments, a self-cleaving peptide is P2A. In some embodiments, a self-cleaving peptide is derived from porcine teschovirus-1. In some embodiments, a self-cleaving peptide is T2A. In some embodiments, a self-cleaving peptide is derived from thosea asigna virus. In some embodiments, a self-cleaving peptide has a sequence listed in Table 8.

In an embodiment, expression sequences encoding peptides separated by a cleavage site have the same level of protein expression.

In some embodiments, a self-cleaving peptide is described in Liu, Z., Chen, O., Wall, J. B. J. et al. Systematic comparison of 2A peptides for cloning multi-genes in a polycistronic vector. Sci Rep 7, 2193 (2017).

### Production of Polynucleotides

The vectors provided herein can be made using standard molecular biology techniques known to persons of skill in the art. For example, the various elements of the vectors provided herein can be obtained using recombinant methods, such as by screening cDNA and genomic libraries from cells, or by deriving the polynucleotides from a vector known to include the same.

The various elements of the vectors provided herein can also be produced synthetically, rather than cloned, based on the known sequences. The complete sequence can be assembled from overlapping oligonucleotides prepared by standard methods and assembled into the complete sequence. See, e.g., Edge, Nature (1981) 292:756; Nambair el al., Science (1984) 223:1299; and Jay et al., J. Biol. Chem. (1984) 259:631 1.

Thus, particular nucleotide sequences can be obtained from vectors harboring the desired sequences or synthesized completely, or in part, using various oligonucleotide synthesis techniques known in the art, such as site-directed mutagenesis and polymerase chain reaction (PCR) techniques where appropriate. One method of obtaining nucleotide sequences encoding the desired vector elements is by annealing complementary sets of overlapping synthetic oligonucleotides produced in a conventional, automated polynucleotide synthesizer, followed by ligation with an appropriate DNA ligase and amplification of the ligated nucleotide sequence via PCR. See, e.g., Jayaraman et al., Proc. Natl. Acad. Sci. USA (1991) 88:4084-4088. Additionally, oligonucleotide-directed synthesis (Jones et al., Nature (1986) 54.75-82), oligonucleotide directed mutagenesis of preexisting nucleotide regions (Riechmann et al., Nature (1988) 332:323-327 and Verhoeyen et al., Science (1988) 239:1534-1536), and enzymatic filling-in of gapped oligonucleotides using T4 DNA polymerase (Queen et al., Proc. Natl. Acad. Sci. USA (1989) 86:10029-10033) can be used.

The precursor RNA provided herein can be generated by incubating a vector provided herein under conditions permissive of transcription of the precursor RNA encoded by the vector. For example, in some embodiments a precursor RNA is synthesized by incubating a vector provided herein that comprises an RNA polymerase promoter upstream of its 5′ duplex forming region and/or expression sequences with a compatible RNA polymerase enzyme under conditions permissive of in vitro transcription. In some embodiments, the vector is incubated inside of a cell by a bacteriophage RNA polymerase or in the nucleus of a cell by host RNA polymerase II.

In certain embodiments, provided herein is a method of generating precursor RNA by performing in vitro transcription using a vector provided herein as a template (e.g., a vector provided herein with a RNA polymerase promoter positioned upstream of the 5′ duplex forming region).

In certain embodiments, the resulting precursor RNA can be used to generate circular RNA (e.g., a circular RNA polynucleotide provided herein) by incubating it in the presence of magnesium ions and guanosine nucleotide or nucleoside at a temperature at which RNA circularization occurs (e.g., between 20° C. and 60° C.).

Thus, in certain embodiments provided herein is a method of making circular RNA. In certain embodiments, the method comprises synthesizing precursor RNA by transcription (e.g., run-off transcription) using a vector provided herein (e.g., a vector comprising, in the following order, a 5′ duplex forming region, a 3′ group I intron fragment, a first spacer, an Internal Ribosome Entry Site (IRES), a first expression sequence, a polynucleotide sequence encoding a cleavage site, a second expression sequence, a second spacer, a 5′ group I intron fragment, and a 3′ duplex forming region) as a template, and incubating the resulting precursor RNA in the presence of divalent cations (e.g., magnesium ions) and GTP such that it circularizes to form circular RNA. In some embodiments, an inventive precursor RNA is capable of circularizing in the absence of magnesium ions and GTP and/or without the step of incubation with magnesium ions and GTP. In some embodiments, transcription is carried out in the presence of an excess of GMP.

In some embodiments, a composition comprising circular RNA has been purified. Circular RNA may be purified by any known method commonly used in the art, such as column chromatography, gel filtration chromatography, and size exclusion chromatography. In some embodiments, purification comprises one or more of the following steps: phosphatase treatment, HPLC size exclusion purification, and RNase R digestion. In some embodiments, purification comprises the following steps in order: RNase R digestion, phosphatase treatment, and HPLC size exclusion purification. In some embodiments, purification comprises reverse phase HPLC. In some embodiments, a purified composition contains less double stranded RNA, DNA splints, triphosphorylated RNA, phosphatase proteins, protein ligases, capping enzymes and/or nicked RNA than unpurified RNA. In some embodiments, a purified composition is less immunogenic than an unpurified composition. In some embodiments, immune cells exposed to a purified composition produce less TNFα, RIG-I, IL-2, IL-6, IFNγ, and/or a type 1 interferon, e.g., IFN-β, than immune cells exposed to an unpurified composition.

### Nanoparticles

In certain aspects, provided herein are pharmaceutical compositions comprising the circular RNA provided herein. In certain embodiments, such pharmaceutical compositions are formulated with nanoparticles to facilitate delivery.

In certain embodiments, the circular RNA provided herein may be delivered and/or targeted to a cell in a transfer vehicle, e.g., a nanoparticle, or a composition comprising a nanoparticle. In some embodiments, the circular RNA may also be delivered to a subject in a transfer vehicle or a composition comprising a transfer vehicle. In some embodiments, the transfer vehicle is a nanoparticle. In some embodiments, the nanoparticle is a lipid nanoparticle, a solid lipid nanoparticle, a polymeric core-shell nanoparticle, or a biodegradable nanoparticle. In some embodiments, the transfer vehicle comprises or is coated with one or more cationic lipids, non-cationic lipids, ionizable lipids, PEG-modified lipids, polyglutamic acid polymers, Hyaluronic acid polymers, poly β-amino esters, poly beta amino peptides, or positively charged peptides.

In one embodiment, the transfer vehicle may be selected and/or prepared to optimize delivery of the circular RNA to a target cell. For example, if the target cell is an antigen presenting cell, the properties of the transfer vehicle (e.g., size, charge and/or pH) may be optimized to effectively deliver such transfer vehicle to the target cell, reduce immune clearance and/or promote retention in that target cell.

The use of transfer vehicles to facilitate the delivery of nucleic acids to target cells is contemplated by the present invention. Liposomes (e.g., liposomal lipid nanoparticles) are generally useful in a variety of applications in research, industry, and medicine, particularly for their use as transfer vehicles of diagnostic or therapeutic compounds in vivo (Lasic, Trends Biotechnol., 16: 307-321, 1998; Drummond et al., Pharmacol. Rev., 51: 691-743, 1999) and are usually characterized as microscopic vesicles having an interior aqueous space sequestered from an outer medium by a membrane of one or more bilayers. Bilayer membranes of liposomes are typically formed by amphiphilic molecules, such as lipids of synthetic or natural origin that comprise spatially separated hydrophilic and hydrophobic domains (Lasic, Trends Biotechnol., 16: 307-321, 1998). Bilayer membranes of the liposomes can also be formed by amphiphilic polymers and surfactants (e.g., polymerosomes, niosomes, etc.).

In the context of the present invention, a transfer vehicle typically serves to transport the circular RNA to the target cell. For the purposes of the present invention, the transfer vehicles are prepared to contain or encapsulate the desired nucleic acids. The process of incorporation of a desired entity (e.g., a nucleic acid) into a liposome is often referred to as loading (Lasic, et al., FEBS Lett., 312: 255-258, 1992). The liposome-incorporated nucleic acids may be completely or partially located in the interior space of the liposome, within the bilayer membrane of the liposome, or associated with the exterior surface of the liposome membrane. The purpose of incorporating a circular RNA into a transfer vehicle, such as a liposome, is often to protect the nucleic acid from an environment which may contain enzymes or chemicals that degrade nucleic acids and/or systems or receptors that cause the rapid excretion of the nucleic acids. Accordingly, in an embodiment of the present invention, the selected transfer vehicle is capable of enhancing the stability of the circular RNA contained therein. The liposome can allow the encapsulated circRNA to reach the target cell, or alternatively limit the delivery of such circular RNA to other sites or cells where the presence of the administered circular RNA may be useless or undesirable. Furthermore, incorporating the circular RNA into a transfer vehicle, such as, for example, a cationic liposome, also facilitates the delivery of such circRNA into a target cell. In some embodiments, a transfer vehicle disclosed herein may serve to promote endosomal or lysosomal release of, for example, contents that are encapsulated in the transfer vehicle (e.g., lipid nanoparticle).

Ideally, transfer vehicles are prepared to encapsulate one or more desired circular RNA such that the compositions demonstrate a high transfection efficiency and enhanced stability. While liposomes can facilitate introduction of nucleic acids into target cells, the addition of polycations (e.g., poly L-lysine and protamine), as a copolymer can in some instances markedly enhance the transfection efficiency of several types of cationic liposomes by 2-28 fold in a number of cell lines both in vitro and in vivo. (See N J. Caplen, et al., Gene Ther. 1995; 2: 603; S. Li, el al., Gene Ther. 1997: 4, 891.)

In some embodiments of the present invention, the transfer vehicle is formulated as a lipid nanoparticle. In an embodiment, the lipid nanoparticles are formulated to deliver one or more circRNA to one or more target cells. Examples of suitable lipids include the phosphatidyl compounds (e.g., PBAE, polyglutamic acid, polyaspartic acid, phosphatidylglycerol, phosphatidylcholine, phosphatidylserine, phosphatidylethanolamine, sphingolipids, cerebrosides, and gangliosides). Also contemplated is the use of polymers as transfer vehicles, whether alone or in combination with other transfer vehicles. Suitable polymers may include, for example, polyacrylates, polyalkycyanoacrylates, polylactide, polylactide-polyglycolide copolymers, polycaprolactones, dextran, albumin, gelatin, alginate, collagen, chitosan, cyclodextrins, dendrimers and polyethylenimine. In some embodiments, the transfer vehicle is formulated as a lipid as described in U.S. patent application Ser. No. 16/065,067, incorporated herein in its entirety. In some embodiments, the transfer vehicle is selected based upon its ability to facilitate the transfection of a circRNA to a target cell.

The invention contemplates the use of lipid nanoparticles as transfer vehicles comprising a cationic lipid to load and/or encapsulate and/or enhance the delivery of circRNA into the target cell that will act as a depot for protein production. The contemplated lipid nanoparticles may be prepared by including multi-component lipid mixtures of varying ratios employing one or more cationic lipids, non-cationic lipids and PEG-modified lipids. Several cationic lipids have been described in the literature, many of which are commercially available.

Suitable cationic lipids for use in the compositions and methods of the invention include those described in International Patent Publication No. WO 2010/053572 and/or U.S. patent application Ser. No. 15/809,680, e.g., C12-200. In certain embodiments, the compositions and methods of the invention employ a lipid nanoparticles comprising an ionizable cationic lipid described in U.S. provisional patent application 61/617,468, filed Mar. 29, 2012 (incorporated herein by reference), such as, e.g, (15Z,18Z)-N,N-dimethyl-6-(9Z,12Z)-octadeca-9,12-dien-1-yl)tetracosa-15,18-dien-1-amine (HGT5000), (15Z,18Z)-N,N-dimethyl-6-((9Z,12Z)-octadeca-9,12-dien-1-yl)tetracosa-4,15,18-trien-1-amine (HGT5001), and (15Z,18Z)-N,N-dimethyl-6-((9Z,12Z)-octadeca-9,12-dien-1-yl)tetracosa-5,15,18-trien-1-amine (HGT5002).

In some embodiments, the cationic lipid N-[1-(2,3-dioleyloxy)propyl]-N,N,N-trimethylammonium chloride or “DOTMA” is used. (Felgner et al., Proc. Nat'l Acad. Sci. 84, 7413 (1987); U.S. Pat. No. 4,897,355). DOTMA can be formulated alone or can be combined with the neutral lipid, dioleoylphosphatidyl-ethanolamine or “DOPE” or other cationic or non-cationic lipids into a transfer vehicle or a lipid nanoparticle, and such liposomes can be used to enhance the delivery of nucleic acids into target cells. Other suitable cationic lipids include, for example, 5-carboxyspermylglycinedioctadecylamide or “DOGS,” 2,3-dioleyloxy-N-[2(spermine-carboxamido)ethyl]-N,N-dimethyl-1-propanaminium or “DOSPA” (Behr el al., Proc. Nat.'l Acad. Sci. 86, 6982 (1989); U.S. Pat. Nos. 5,171,678; 5,334,761), 1,2-Dioleoyl-3-Dimethylammonium-Propane or “DODAP,” 1,2-Dioleoyl-3-Trimethylammonium-Propane or “DOTAP.” Contemplated cationic lipids also include 1,2-distearyloxy-N,N-dimethyl-3-aminopropane or “DSDMA”, 1,2-dioleyloxy-N,N-dimethyl-3-aminopropane or “DODMA,” 1,2-dilinoleyloxy-N,N-dimethyl-3-aminopropane or “DLinDMA,” 1,2-dilinolenyloxy-N,N-dimethyl-3-aminopropane or “DLenDMA,” N-dioleyl-N,N-dimethylammonium chloride or “DODAC,” N,N-distearyl-N,N-dimethylammonium bromide or “DDAB,” N-(1,2-dimyristyloxyprop-3-yl)-N,N-dimethyl-N-hydroxyethyl ammonium bromide or “DMRIE,” 3-dimethylamino-2-(cholest-5-en-3-beta-oxybutan-4-oxy)-1-(cis,cis-9,12-octadecadienoxy)propane or “CLinDMA,” 2-[5′-(cholest-5-en-3-beta-oxy)-3′-oxapentoxy)-3-dimethy 1-1-(cis,cis-9′, 1-2′-octadecadienoxy)propane or “CpLinDMA,” N,N-dimethyl-3,4-dioleyloxybenzylamine or “DMOBA,” 1,2-N,N′-dioleylcarbamyl-3-dimethylaminopropane or “DOcarbDAP,” 2,3-Dilinoleoyloxy-N,N-dimethylpropylamine or “DLinDAP,” 1,2-N,N′-Dilinoleylcarbamyl-3-dimethylaminopropane or “DLincarbDAP,” 1,2-Dilinoleoylcarbamyl-3-dimethylaminopropane or “DLinCDAP,” 2,2-dilinoleyl-4-dimethylaminomethyl-[1,3]-dioxolane or “DLin-K-DMA,” 2,2-dilinoleyl-4-dimethylaminoethyl-[1,3]-dioxolane or “DLin-K-XTC2-DMA,” and 2-(2,2-di((9Z,12Z)-octadeca-9,12-dien-1-yl)-1,3-dioxolan-4-yl)-N,N-dimethylethanamine (DLin-KC2-DMA)) (See, WO 2010/042877; Semple et al., Nature Biotech. 28:172-176 (2010)), or mixtures thereof. (Heyes, J., et al., J Controlled Release 107: 276-287 (2005); Morrissey, D. V., et al., Nat. Biotechnol. 23(8): 1003-1007 (2005); PCT Publication WO2005/121348A1).

The use of cholesterol-based cationic lipids is also contemplated by the present invention. Such cholesterol-based cationic lipids can be used, either alone or in combination with other cationic or non-cationic lipids. Suitable cholesterol-based cationic lipids include, for example, GL67, DC-Chol (N,N-dimethyl-N-ethylcarboxamidocholesterol), 1,4-bis(3-N-oleylamino-propyl)piperazine (Gao, et al., Biochem. Biophys. Res. Comm. 179, 280 (1991); Wolf et al., BioTechniques 23, 139 (1997); U.S. Pat. No. 5,744,335), or ICE.

In addition, several reagents are commercially available to enhance transfection efficacy. Suitable examples include LIPOFECTIN (DOTMA:DOPE) (Invitrogen, Carlsbad, CA), LIPOFECTAMINE (DOSPA:DOPE) (Invitrogen), LIPOFECTAMINE2000. (Invitrogen), FUGENE (Promega, Madison, WI), TRANSFECTAM (DOGS) (Promega), and EFFECTENE (Qiagen, Valencia, CA).

Also contemplated are cationic lipids such as the dialkylamino-based, imidazole-based, and guanidinium-based lipids, such as those described in U.S. Pat. No. 10,413,618.

In other embodiments, the compositions and methods described herein are directed to lipid nanoparticles comprising one or more cleavable lipids, such as, for example, one or more cationic lipids or compounds that comprise a cleavable disulfide (S—S) functional group (e.g., HGT4001, HGT4002, HGT4003, HGT4004 and HGT4005), as further described in U.S. Provisional Application No. 61/494,745, the entire teachings of which are incorporated herein by reference in their entirety.

The use of polyethylene glycol (PEG)-modified phospholipids and derivatized lipids such as derivatized ceramides (PEG-CER), including N-Octanoyl-Sphingosine-1-[Succinyl(Methoxy Polyethylene Glycol)-2000] (C8 PEG-2000 ceramide) is also contemplated by the present invention, either alone or in combination with other lipids, together which comprise the transfer vehicle (e.g., a lipid nanoparticle). Contemplated PEG-modified lipids include, but are not limited to, a polyethylene glycol chain of up to 5 kDa in length covalently attached to a lipid with alkyl chain(s) of C6-C20 length. The addition of such components may prevent complex aggregation and may also provide a means for increasing circulation lifetime and increasing the delivery of the lipid-nucleic acid composition to the target cell, (Klibanov et al., (1990) FEBS Letters, 268 (1): 235-237), or they may be selected to rapidly exchange out of the formulation in vivo (see U.S. Pat. No. 5,885,613). Particularly useful exchangeable lipids are PEG-ceramides having shorter acyl chains (e.g., C14 or C18). The PEG-modified phospholipid and derivatized lipids of the present invention may comprise a molar ratio from about 0% to about 20%, about 0.5% to about 20%, about 1% to about 15%, about 4% to about 10%, or about 2% of the total lipid present in the transfer vehicle. PEG end groups are contemplated herein. In some embodiments, a PEG end group is —OH, —OCH₃, an acid, an amine, or a guanidine.

In some embodiments, the RNA (e.g., circRNA) vaccine may be associated with a cationic or polycationic compounds, including protamine, nucleoline, spermine or spermidine, or other cationic peptides or proteins, such as poly-L-lysine (PLL), polyarginine, basic polypeptides, cell penetrating peptides (CPPs), including HIV-binding peptides, HIV-1 Tat (HIV), Tat-derived peptides, Penetratin, VP22 derived or analog peptides, Pestivirus Erns, HSV, VP22 (Herpes simplex), MAP, KALA or protein transduction domains (PTDs), PpT620, prolin-rich peptides, arginine-rich peptides, lysine-rich peptides, MPG-peptide(s), Pep-1, L-oligomers, Calcitonin peptide(s), Antennapedia-derived peptides (particularly from Drosophila antennapedia), pAntp, plsl, FGF, Lactoferrin, Transportan, Buforin-2, Bac715-24, SynB, SynB(1), pVEC, hCT-derived peptides, SAP, histones, cationic polysaccharides, for example chitosan, polybrene, cationic polymers, e.g. polyethyleneimine (PEI), cationic lipids, e.g. DOTMA: [1-(2,3-sioleyloxy)propyl)]-N,N,N-trimethylammonium chloride, DMRIE, di-C14-amidine, DOTIM, SAINT, DC-Chol, BGTC, CTAP, DOPC, DODAP, DOPE: Dioleyl phosphatidylethanol-amine, DOSPA, DODAB, DOIC, DMEPC, DOGS: Dioctadecylamidoglicylspermin, DIMRI: Dimyristooxypropyl dimethyl hydroxyethyl ammonium bromide, DOTAP: dioleoyloxy-3-(trimethylammonio)propane, DC-6-14; O,O-ditetradecanoyl-N-.alpha.-trimethylammonioacetyl)diethanolamine chloride, CLIP 1: rac-[(2,3-dioctadecyloxypropyl)(2-hydroxyethyl)]-dimethylammonium chloride, CLIP6: rac-[2(2,3-dihexadecyloxypropyloxymethyloxy)ethyl]-trimethylammonium, CLIP9: rac-[2(2,3-dihexadecyloxypropyloxysuccinyloxy)ethyl]-trimethylammonium, oligofectamine, or cationic or polycationic polymers, e.g. modified polyaminoacids, such as beta-aminoacid-polymers or reversed polyamides, etc., modified polyethylenes, such as PVP (poly(N-ethyl-4-vinylpyridinium bromide)), etc., modified acrylates, such as pDMAEMA (poly(dimethylaminoethyl methylacrylate)), etc., modified amidoamines such as pAMAM (poly(amidoamine)), etc., modified polybetaminoester (PBAE), such as diamine end modified 1,4 butanediol diacrylate-co-5-amino-1-pentanol polymers, etc., dendrimers, such as polypropylamine dendrimers or pAMAM based dendrimers, etc., polyimine(s), such as PEI; poly(ethyleneimine), poly(propyleneimine), etc., polyallylamine, sugar backbone based polymers, such as cyclodextrin based polymers, dextran based polymers, chitosan, etc., silan backbone based polymers, such as PMOXA-PDMS copolymers, etc., blockpolymers consisting of a combination of one or more cationic blocks (e.g. selected from a cationic polymer as mentioned above) and of one or more hydrophilic or hydrophobic blocks (e.g. polyethyleneglycole), etc.

The present invention also contemplates the use of non-cationic lipids including those described in U.S. patent application Ser. No. 15/809,680. Non-cationic lipids include, but are not limited to, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoylphosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 16-O-monomethyl PE, 16-O-dimethyl PE, 18-1-trans PE, 1-stearoyl-2-oleoyl-phosphatidyethanolamine (SOPE), cholesterol, or a mixture thereof. Such non-cationic lipids may be used alone or in combination with other excipients, for example, cationic lipids. When used in combination with a cationic lipid, the non-cationic lipid may comprise a molar ratio of 5% to about 90%, or about 10% to about 70% of the total lipid present in the transfer vehicle.

The transfer vehicle (e.g., a lipid nanoparticle) may be prepared by combining multiple lipid and/or polymer components. For example, a transfer vehicle may be prepared using C12-200, DOPE, cholesterol, DMG-PEG2K at a molar ratio of 40:30:25:5, or DODAP, DOPE, cholesterol, DMG-PEG2K at a molar ratio of 18:56:20:6, or HGT5000, DOPE, cholesterol, DMG-PEG2K at a molar ratio of 40:20:35:5, or HGT5001, DOPE, cholesterol, DMG-PEG2K at a molar ratio of 40:20:35:5. The selection of cationic lipids, non-cationic lipids and/or PEG-modified lipids which comprise the lipid nanoparticle, as well as the relative molar ratio of such lipids to each other, is based upon the characteristics of the selected lipid(s), the nature of the intended target cells, the characteristics of the circRNA to be delivered. Additional considerations include, for example, the saturation of the alkyl chain, as well as the size, charge, pH, pKa, fusogenicity and toxicity of the selected lipid(s). Thus, the molar ratios may be adjusted accordingly. For example, in some embodiments, the percentage of cationic lipid in the lipid nanoparticle may be greater than 10%, greater than 20%, greater than 30%, greater than 40%, greater than 50%, greater than 60%, or greater than 70%. The percentage of non-cationic lipid in the lipid nanoparticle may be greater than 5%, greater than 10%, greater than 20%, greater than 30%, or greater than 40%. The percentage of cholesterol in the lipid nanoparticle may be greater than 10%, greater than 20%, greater than 30%, or greater than 40%. The percentage of PEG-modified lipid in the lipid nanoparticle may be greater than 1%, greater than 2%, greater than 5%, greater than 10%, or greater than 20%.

The transfer vehicles for use in the compositions of the invention can be prepared by various techniques which are presently known in the art. Multi-lamellar vesicles (MLV) may be prepared using conventional techniques, for example, by depositing a selected lipid on the inside wall of a suitable container or vessel, dissolving the lipid in an appropriate solvent, and then evaporating the solvent to leave a thin film on the inside of the vessel or by spray-drying. An aqueous phase may then be added to the vessel with a vortexing motion, which results in the formation of MLVs. Uni-lamellar vesicles (ULV) can then be formed by homogenization, sonication or extrusion of the multi-lamellar vesicles. In addition, ULV can be formed by detergent removal techniques.

In certain embodiments of this invention, the compositions of the present invention comprise a transfer vehicle wherein the circRNA is associated on both the surface of the transfer vehicle and encapsulated within the same transfer vehicle. For example, during preparation of the compositions of the present invention, cationic transfer vehicles may associate with the circRNA through electrostatic interactions.

In certain embodiments, the compositions of the invention may be loaded with diagnostic radionuclide, fluorescent materials or other materials that are detectable in both in vitro and in vivo applications. For example, suitable diagnostic materials for use in the present invention may include Rhodamine-dioleoylphospha-tidylethanolamine (Rh-PE), Green Fluorescent Protein circRNA (GFP circRNA), Renilla Luciferase circRNA and Firefly Luciferase circRNA.

In some embodiments, selection of the appropriate size of a transfer vehicle takes into consideration the site of the target cell or tissue and, to some extent, the application for which the liposome is being made. In some embodiments, it may be desirable to limit transfection of the circRNA to certain cells or tissues. For example, to target hepatocytes, a transfer vehicle may be sized such that its dimensions are smaller than the fenestrations of the endothelial layer lining hepatic sinusoids in the liver. Accordingly, the appropriately-sized transfer vehicle can readily penetrate such endothelial fenestrations to reach the target hepatocytes. Alternatively, a transfer vehicle may be sized such that the dimensions of the liposome are of a sufficient diameter to limit or expressly avoid distribution into certain cells or tissues. For example, a transfer vehicle may be sized such that its dimensions are larger than the fenestrations of the endothelial layer lining hepatic sinusoids to thereby limit distribution of the transfer vehicle to hepatocytes. Generally, the size of the transfer vehicle is within the range of about 25 to 250 nm. In some embodiments, the size of the transfer vehicle is less than about 250 nm, 175 nm, 150 nm, 125 nm, 100 nm, 75 nm, 50 nm, 25 nm or 10 nm.

A variety of alternative methods known in the art are available for sizing of a population of transfer vehicles. One such sizing method is described in U.S. Pat. No. 4,737,323, incorporated herein by reference. Sonicating a liposome suspension either by bath or probe sonication produces a progressive size reduction down to small ULV less than about 0.05 microns in diameter. Homogenization is another method that relies on shearing energy to fragment large liposomes into smaller ones. In a typical homogenization procedure, MLV are recirculated through a standard emulsion homogenizer until selected liposome sizes, typically between about 0.1 and 0.5 microns, are observed. The size of the liposomal vesicles may be determined by quasi-electric light scattering (QELS) as described in Bloomfield, Ann. Rev. Biophys. Bioeng., 10:421-450 (1981), incorporated herein by reference. Average liposome diameter may be reduced by sonication of formed liposomes. Intermittent sonication cycles may be alternated with QELS assessment to guide efficient liposome synthesis.

Additionally, in certain embodiments, the circular RNA provided herein can be formulated using one or more liposomes, lipoplexes, or lipid nanoparticles. In one embodiment, the circular RNA may be formulated in a lipid nanoparticle such as those described in International Publication No. WO2012170930, herein incorporated by reference in its entirety. In one embodiment, the lipid may be a cleavable lipid such as those described in International Publication No. WO2012170889, herein incorporated by reference in its entirety. In one embodiment, the pharmaceutical compositions of the circular RNA may include at least one of the PEGylated lipids described in International Publication No. WO2012099755, herein incorporated by reference. In one embodiment, a lipid nanoparticle formulation may be formulated by the methods described in International Publication Nos. WO2011127255 or WO2008103276, each of which is herein incorporated by reference in their entirety. A lipid nanoparticle may be coated or associated with a co-polymer such as, but not limited to, a block co-polymer, such as a branched polyether-polyamide block copolymer described in International Publication No. WO2013012476, herein incorporated by reference in its entirety. Liposomes, lipoplexes, or lipid nanoparticles may be used to improve the efficacy of circular RNA directed protein production, as these formulations may be able to increase cell transfection by the circular RNA, increase the in vivo or in vitro half-life of the circular RNA, and/or allow for controlled release.

In other embodiments, the circular RNA polynucleotide provided herein can be formulated using one or more polymers. A polymer may be included in and/or used to encapsulate or partially encapsulate the RNA or a lipid nanoparticle. A polymer may be biodegradable and/or biocompatible. A polymer may be selected from, but is not limited to, polyamines, polyethers, polyamides, polyesters, polycarbamates, polyureas, polycarbonates, polystyrenes, polyimides, polysulfones, polyurethanes, polyacetylenes, polyethylenes, polyethyleneimines, polyisocyanates, polyacrylates, polymethacrylates, polyacrylonitriles, and polyarylates. For example, a polymer may include poly(caprolactone) (PCL), ethylene vinyl acetate polymer (EVA), poly(lactic acid) (PLA), poly(L-lactic acid) (PLLA), poly(glycolic acid) (PGA), poly(lactic acid-co-glycolic acid) (PLGA), poly(L-lactic acid-co-glycolic acid) (PLLGA), poly(D,L-lactide) (PDLA), poly(Llactide) (PLLA), poly(D,L-lactide-co-caprolactone), poly(D,L-lactide-co-caprolactone-coglycolide), poly(D,L-lactide-co-PEO-co-D,L-lactide), poly(D,L-lactide-co-PPO-co-D,L-lactide), polyalkyl cyanoacrylate, polyurethane, poly-L-lysine (PLL), hydroxypropyl methacrylate (HPMA), polyethyleneglycol, poly-L-glutamic acid, poly(hydroxy acids), polyanhydrides, polyorthoesters, poly(ester amides), polyamides, poly(ester ethers), polycarbonates, polyalkylenes such as polyethylene and polypropylene, polyalkylene glycols such as poly(ethylene glycol) (PEG), polyalkylene oxides (PEO), polyalkylene terephthalates such as poly(ethylene terephthalate), polyvinyl alcohols (PVA), polyvinyl ethers, polyvinyl esters such as poly(vinyl acetate), polyvinyl halides such as poly(vinyl chloride) (PVC), polyvinylpyrrolidone (PVP), polysiloxanes, polystyrene, polyurethanes, derivatized celluloses such as alkyl celluloses, hydroxyalkyl celluloses, cellulose ethers, cellulose esters, nitro celluloses, hydroxypropylcellulose, carboxymethylcellulose, polymers of acrylic acids, such as poly(methyl(meth)acrylate) (PMMA), poly(ethyl(meth)acrylate), poly(butyl(meth)acrylate), 363 5 10 15 20 25 30 35 WO 2021/076805 PCT/US2020/055844 poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), poly(octadecyl acrylate) and copolymers and mixtures thereof, polydioxanone and its copolymers, polyhydroxyalkanoates, polypropylene fiimarate, polyoxymethylene, poloxamers, poloxamines, poly(ortho)esters, poly(butyric acid), poly(valeric acid), poly(lactide-co-caprolactone), trimethylene carbonate, poly(N-acryloylmorpholine) (PAcM), poly(2-methyl-2-oxazoline) (PMOX), poly(2-ethyl-2-oxazoline) (PEOZ), and polyglycerol.

In some embodiments, a polynucleotide encodes a protein that is made up of subunits that are encoded by more than one gene. For example, the protein may be a heterodimer, wherein each chain or subunit of the protein is encoded by a separate gene. It is possible that more than one circRNA molecule is delivered in the transfer vehicle and each circRNA encodes a separate subunit of the protein. Alternatively, a single circRNA may be engineered to encode more than one subunit (e.g., in the case of a single-chain Fv antibody). In certain embodiments, separate circRNA molecules encoding the individual subunits may be administered in separate transfer vehicles.

The present invention also contemplates the discriminatory targeting of target cells and tissues by both passive and active targeting means. The phenomenon of passive targeting exploits the natural distributions patterns of a transfer vehicle in vivo without relying upon the use of additional excipients or means to enhance recognition of the transfer vehicle by target cells. For example, transfer vehicles which are subject to phagocytosis by the cells of the reticulo-endothelial system are likely to accumulate in the liver or spleen and, accordingly, may provide a means to passively direct the delivery of the compositions to such target cells.

Alternatively, the present invention contemplates active targeting, which involves the use of targeting moieties that may be bound (either covalently or non-covalently) to the transfer vehicle to encourage localization of such transfer vehicle to certain target cells or target tissues. For example, targeting may be mediated by the inclusion of one or more endogenous targeting moieties in or on the transfer vehicle to encourage distribution to the target cells or tissues. Recognition of the targeting moiety by the target tissues actively facilitates tissue distribution and cellular uptake of the transfer vehicle and/or its contents in the target cells and tissues (e.g., the inclusion of an apolipoprotein-E targeting ligand in or on the transfer vehicle encourages recognition and binding of the transfer vehicle to endogenous low density lipoprotein receptors expressed by hepatocytes). As provided herein, the composition can comprise a moiety capable of enhancing affinity of the composition to the target cell. Targeting moieties may be linked to the outer bilayer of the lipid particle during formulation or post-formulation. These methods are well known in the art. In addition, some lipid particle formulations may employ fusogenic polymers such as PEAA, hemagluttinin, other lipopeptides (see U.S. patent application Ser. No. 08/835,281, and 60/083,294, which are incorporated herein by reference) and other features useful for in vivo and/or intracellular delivery. In some embodiments, the compositions of the present invention demonstrate improved transfection efficacies, and/or demonstrate enhanced selectivity towards target cells or tissues of interest. Contemplated therefore are compositions which comprise one or more moieties (e.g., peptides, aptamers, oligonucleotides, small molecules, a vitamin or other molecules) that are capable of enhancing the affinity of the compositions and their nucleic acid contents for the target cells or tissues. Suitable moieties may optionally be bound or linked to the surface of the transfer vehicle. In some embodiments, the targeting moiety may span the surface of a transfer vehicle or be encapsulated within the transfer vehicle. Suitable moieties are selected based upon their physical, chemical or biological properties (e.g., selective affinity and/or recognition of target cell surface markers or features). Cell-specific target sites and their corresponding targeting ligand can vary widely. Suitable targeting moieties are selected such that the unique characteristics of a target cell are exploited, thus allowing the composition to discriminate between target and non-target cells. For example, compositions of the invention may include surface markers (e.g., apolipoprotein-B or apolipoprotein-E) that selectively enhance recognition of, or affinity to hepatocytes (e.g., by receptor-mediated recognition of and binding to such surface markers). As an example, the use of galactose as a targeting moiety would be expected to direct the compositions of the present invention to parenchymal hepatocytes, or alternatively the use of mannose containing sugar residues as a targeting ligand would be expected to direct the compositions of the present invention to liver endothelial cells (e.g., mannose containing sugar residues that may bind preferentially to the asialoglycoprotein receptor present in hepatocytes). (See Hillery A M, et al., “Drug Delivery and Targeting: For Pharmacists and Pharmaceutical Scientists” (2002) Taylor & Francis, Inc.) The presentation of such targeting moieties that have been conjugated to moieties present in the transfer vehicle (e.g., a lipid nanoparticle) therefore facilitate recognition and uptake of the compositions of the present invention in target cells and tissues. Examples of suitable targeting moieties include one or more peptides, proteins, small molecules, aptamers, vitamins and oligonucleotides.

In some embodiments, the targeting moiety mediates receptor-mediated endocytosis selectively into a specific population of cells. In some embodiments, the targeting moiety is capable of binding to a hepatic cell antigen. In some embodiments, the targeting moiety is a single chain variable fragment (scFv), nanobody, peptide, peptide-based macrocycle, minibody, heavy chain variable region, light chain variable region or fragment thereof.

In some embodiments, circular RNA is formulated according to a process described in U.S. patent application Ser. No. 15/809,680. In some embodiments, the present invention provides a process of encapsulating circular RNA in lipid nanoparticles comprising the steps of forming lipids into pre-formed lipid nanoparticles (i.e. formed in the absence of RNA) and then combining the pre-formed lipid nanoparticles with RNA. In some embodiments, the novel formulation process results in an RNA formulation with higher potency (peptide or protein expression) and higher efficacy (improvement of a biologically relevant endpoint) both in vitro and in vivo with potentially better tolerability as compared to the same RNA formulation prepared without the step of preforming the lipid nanoparticles (e.g., combining the lipids directly with the RNA).

For certain cationic lipid nanoparticle formulations of RNA, in order to achieve high encapsulation of RNA, the RNA in buffer (e.g., citrate buffer) has to be heated. In those processes or methods, the heating is required to occur before the formulation process (i.e. heating the separate components) as heating post-formulation (post-formation of nanoparticles) does not increase the encapsulation efficiency of the RNA in the lipid nanoparticles. In contrast, in some embodiments of the processes of the present invention, the order of heating of RNA does not appear to affect the RNA encapsulation percentage. In some embodiments, no heating (i.e. maintaining at ambient temperature) of one or more of the solution comprising the pre-formed lipid nanoparticles, the solution comprising the RNA and the mixed solution comprising the lipid nanoparticle encapsulated RNA is required to occur before or after the formulation process.

RNA may be provided in a solution to be mixed with a lipid solution such that the RNA may be encapsulated in lipid nanoparticles. A suitable RNA solution may be any aqueous solution containing RNA to be encapsulated at various concentrations. For example, a suitable RNA solution may contain an RNA at a concentration of or greater than about 0.01 mg/ml, 0.05 mg/ml, 0.06 mg/ml, 0.07 mg/ml, 0.08 mg/ml, 0.09 mg/ml, 0.1 mg/ml, 0.15 mg/ml, 0.2 mg/ml, 0.3 mg/ml, 0.4 mg/ml, 0.5 mg/ml, 0.6 mg/ml, 0.7 mg/ml, 0.8 mg/ml, 0.9 mg/ml, or 1.0 mg/ml. In some embodiments, a suitable RNA solution may contain an RNA at a concentration in a range from about 0.01-1.0 mg/ml, 0.01-0.9 mg/ml, 0.01-0.8 mg/ml, 0.01-0.7 mg/ml, 0.01-0.6 mg/ml, 0.01-0.5 mg/ml, 0.01-0.4 mg/ml, 0.01-0.3 mg/ml, 0.01-0.2 mg/ml, 0.01-0.1 mg/ml, 0.05-1.0 mg/ml, 0.05-0.9 mg/ml, 0.05-0.8 mg/ml, 0.05-0.7 mg/ml, 0.05-0.6 mg/ml, 0.05-0.5 mg/ml, 0.05-0.4 mg/ml, 0.05-0.3 mg/ml, 0.05-0.2 mg/ml, 0.05-0.1 mg/ml, 0.1-1.0 mg/ml, 0.2-0.9 mg/ml, 0.3-0.8 mg/ml, 0.4-0.7 mg/ml, or 0.5-0.6 mg/ml.

Typically, a suitable RNA solution may also contain a buffering agent and/or salt. Generally, buffering agents can include Tris, HEPES, ammonium sulfate, sodium bicarbonate, sodium citrate, sodium acetate, potassium phosphate or sodium phosphate. In some embodiments, a suitable concentration of the buffering agent may be in a range from about 0.1 mM to 100 mM, 0.5 mM to 90 mM, 1.0 mM to 80 mM, 2 mM to 70 mM, 3 mM to 60 mM, 4 mM to 50 mM, 5 mM to 40 mM, 6 mM to 30 mM, 7 mM to 20 mM, 8 mM to 15 mM, or 9 to 12 mM.

Exemplary salts can include sodium chloride, magnesium chloride, and potassium chloride. In some embodiments, suitable concentration of salts in an RNA solution may be in a range from about 1 mM to 500 mM, 5 mM to 400 mM, 10 mM to 350 mM, 15 mM to 300 mM, 20 mM to 250 mM, 30 mM to 200 mM, 40 mM to 190 mM, 50 mM to 180 mM, 50 mM to 170 mM, 50 mM to 160 mM, 50 mM to 150 mM, or 50 mM to 100 mM.

In some embodiments, a suitable RNA solution may have a pH in a range from about 3.5-6.5, 3.5-6.0, 3.5-5.5, 3.5-5.0, 3.5-4.5, 4.0-5.5, 4.0-5.0, 4.0-4.9, 4.0-4.8, 4.0-4.7, 4.0-4.6, or 4.0-4.5.

Various methods may be used to prepare an RNA solution suitable for the present invention. In some embodiments, RNA may be directly dissolved in a buffer solution described herein. In some embodiments, an RNA solution may be generated by mixing an RNA stock solution with a buffer solution prior to mixing with a lipid solution for encapsulation. In some embodiments, an RNA solution may be generated by mixing an RNA stock solution with a buffer solution immediately before mixing with a lipid solution for encapsulation.

According to the present invention, a lipid solution contains a mixture of lipids suitable to form lipid nanoparticles for encapsulation of RNA. In some embodiments, a suitable lipid solution is ethanol based. For example, a suitable lipid solution may contain a mixture of desired lipids dissolved in pure ethanol (i.e. 100% ethanol). In another embodiment, a suitable lipid solution is isopropyl alcohol based. In another embodiment, a suitable lipid solution is dimethylsulfoxide-based. In another embodiment, a suitable lipid solution is a mixture of suitable solvents including, but not limited to, ethanol, isopropyl alcohol and dimethylsulfoxide.

A suitable lipid solution may contain a mixture of desired lipids at various concentrations. In some embodiments, a suitable lipid solution may contain a mixture of desired lipids at a total concentration in a range from about 0.1-100 mg/ml, 0.5-90 mg/ml, 1.0-80 mg/ml, 1.0-70 mg/ml, 1.0-60 mg/ml, 1.0-50 mg/ml, 1.0-40 mg/ml, 1.0-30 mg/ml, 1.0-20 mg/ml, 1.0-15 mg/ml, 1.0-10 mg/ml, 1.0-9 mg/ml, 1.0-8 mg/ml, 1.0-7 mg/ml, 1.0-6 mg/ml, or 1.0-5 mg/ml.

Any desired lipids may be mixed at any ratios suitable for encapsulating RNAs. In some embodiments, a suitable lipid solution contains a mixture of desired lipids including cationic lipids, helper lipids (e.g., non cationic lipids and/or cholesterol lipids) and/or PEGylated lipids. In some embodiments, a suitable lipid solution contains a mixture of desired lipids including one or more cationic lipids, one or more helper lipids (e.g., non cationic lipids and/or cholesterol lipids) and one or more PEGylated lipids.

In some embodiments, the compositions of the invention transfect or distribute to target cells on a discriminatory basis (i.e. do not transfect non-target cells). The compositions of the invention may also be prepared to preferentially target a variety of target cells, which include, but are not limited to, hepatocytes, epithelial cells, hematopoietic cells, epithelial cells, endothelial cells, lung cells, bone cells, stem cells, mesenchymal cells, neural cells (e.g., meninges, astrocytes, motor neurons, cells of the dorsal root ganglia and anterior horn motor neurons), photoreceptor cells (e.g., rods and cones), retinal pigmented epithelial cells, secretory cells, cardiac cells, adipocytes, vascular smooth muscle cells, cardiomyocytes, skeletal muscle cells, beta cells, pituitary cells, synovial lining cells, ovarian cells, testicular cells, fibroblasts, B cells, T cells, antigen presenting cells (e.g., dendritic cells), reticulocytes, leukocytes, granulocytes and tumor cells.

### Pharmaceutical Compositions

In certain embodiments, provided herein are compositions (e.g., pharmaceutical compositions) comprising a therapeutic agent provided herein. In certain embodiments, the therapeutic agent is a RNA polynucleotide provided herein. In some embodiments, the therapeutic agent is a circular RNA polynucleotide provided herein. In some embodiments the therapeutic agent is a vector provided herein. In some embodiments, the therapeutic agent is a cell comprising a RNA polynucleotide, circular RNA, or vector provided herein (e.g., a human cell, such as a human antigen presenting cell). In certain embodiments, the composition further comprises a pharmaceutically acceptable carrier. In some embodiments, the compositions provided herein comprise a therapeutic agent provided herein in combination with other pharmaceutically active agents or drugs, such as anti-inflammatory drugs or antibodies capable of targeting B cell antigens, e.g., anti-CD20 antibodies, e.g., rituximab.

With respect to pharmaceutical compositions, the pharmaceutically acceptable carrier can be any of those conventionally used and is limited only by chemico-physical considerations, such as solubility and lack of reactivity with the active agent(s), and by the route of administration. The pharmaceutically acceptable carriers described herein, for example, vehicles, adjuvants, excipients, and diluents, are well-known to those skilled in the art and are readily available to the public. It is preferred that the pharmaceutically acceptable carrier be one which is chemically inert to the therapeutic agent(s) and one which has no detrimental side effects or toxicity under the conditions of use.

The choice of carrier will be determined in part by the particular therapeutic agent, as well as by the particular method used to administer the therapeutic agent. Accordingly, there are a variety of suitable formulations of the pharmaceutical compositions provided herein.

In certain embodiments, the pharmaceutical composition comprises a preservative. In certain embodiments, suitable preservatives may include, for example, methylparaben, propylparaben, sodium benzoate, and benzalkonium chloride. Optionally, a mixture of two or more preservatives may be used. The preservative or mixtures thereof are typically present in an amount of about 0.0001% to about 2% by weight of the total composition.

In some embodiments, the pharmaceutical composition comprises a buffering agent. In some embodiments, suitable buffering agents may include, for example, citric acid, sodium citrate, phosphoric acid, potassium phosphate, and various other acids and salts. A mixture of two or more buffering agents optionally may be used. The buffering agent or mixtures thereof are typically present in an amount of about 0.001% to about 4% by weight of the total composition.

In some embodiments, the concentration of therapeutic agent in the pharmaceutical composition can vary, e.g., less than about 1%, or at least about 1%, 2%, 3%, 4%, 5%, 6%, 7%, 8%, 9%, 10%, 15%, 20%, 25%, 30%, 35%, 40%, 45%, or about 50% or more by weight, and can be selected primarily by fluid volumes, and viscosities, in accordance with the particular mode of administration selected.

The following formulations for oral, aerosol, parenteral (e.g., subcutaneous, intravenous, intraarterial, intramuscular, intradermal, intraperitoneal, and intrathecal), and topical administration are merely exemplary and are in no way limiting. More than one route can be used to administer the therapeutic agents provided herein, and in certain instances, a particular route can provide a more immediate and more effective response than another route.

Formulations suitable for oral administration can comprise or consist of (a) liquid solutions, such as an effective amount of the therapeutic agent dissolved in diluents, such as water, saline, or orange juice; (b) capsules, sachets, tablets, lozenges, and troches, each containing a predetermined amount of the active ingredient, as solids or granules; (c) powders; (d) suspensions in an appropriate liquid; and (e) suitable emulsions. Liquid formulations may include diluents, such as water and alcohols, for example, ethanol, benzyl alcohol and the polyethylene alcohols, either with or without the addition of a pharmaceutically acceptable surfactant. Capsule forms can be of the ordinary hard or soft shelled gelatin type containing, for example, surfactants, lubricants, and inert fillers, such as lactose, sucrose, calcium phosphate, and corn starch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and other pharmacologically compatible excipients. Lozenge forms can comprise the therapeutic agent with a flavorant, usually sucrose, acacia or tragacanth. Pastilles can comprise the therapeutic agent with an inert base, such as gelatin and glycerin, or sucrose and acacia, emulsions, gels, and the like containing, in addition to, such excipients as are known in the art.

Formulations suitable for parenteral administration include aqueous and nonaqueous isotonic sterile injection solutions, which can contain antioxidants, buffers, bacteriostats, and solutes that render the formulation isotonic with the blood of the intended recipient, and aqueous and nonaqueous sterile suspensions that can include suspending agents, solubilizers, thickening agents, stabilizers, and preservatives. In some embodiments, the therapeutic agents provided herein can be administered in a physiologically acceptable diluent in a pharmaceutical carrier, such as a sterile liquid or mixture of liquids including water, saline, aqueous dextrose and related sugar solutions, an alcohol such as ethanol or hexadecyl alcohol, a glycol such as propylene glycol or polyethylene glycol, dimethylsulfoxide, glycerol, ketals such as 2,2-dimethyl-1,3-dioxolane-4-methanol, ethers, poly(ethyleneglycol) 400, oils, fatty acids, fatty acid esters or glycerides, or acetylated fatty acid glycerides with or without the addition of a pharmaceutically acceptable surfactant such as a soap or a detergent, suspending agent such as pectin, carbomers, methylcellulose, hydroxypropylmethylcellulose, or carboxymethylcellulose, or emulsifying agents and other pharmaceutical adjuvants.

Oils, which can be used in parenteral formulations in some embodiments, include petroleum, animal oils, vegetable oils, or synthetic oils. Specific examples of oils include peanut, soybean, sesame, cottonseed, corn, olive, petrolatum, and mineral oil. Suitable fatty acids for use in parenteral formulations include oleic acid, stearic acid, and isostearic acid. Ethyl oleate and isopropyl myristate are examples of suitable fatty acid esters.

Suitable soaps for use in certain embodiments of parenteral formulations include fatty alkali metal, ammonium, and triethanolamine salts, and suitable detergents include (a) cationic detergents such as, for example, dimethyl dialkyl ammonium halides. and alkyl pyridinium halides, (b) anionic detergents such as, for example, alkyl, aryl, and olefin sulfonates, alky, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-β-aminopropionates, and 2-alkyl-imidazoline quaternary ammonium salts, and (e) mixtures thereof.

In some embodiments, the parenteral formulations will contain, for example, from about 0.5% to about 25% by weight of the therapeutic agent in solution. Preservatives and buffers may be used. In order to minimize or eliminate irritation at the site of injection, such compositions may contain one or more nonionic surfactants having, for example, a hydrophile-lipophile balance (HLB) of from about 12 to about 17. The quantity of surfactant in such formulations will typically range, for example, from about 5% to about 15% by weight. Suitable surfactants include polyethylene glycol, sorbitan, fatty acid esters such as sorbitan monooleate, and high molecular weight adducts of ethylene oxide with a hydrophobic base formed by the condensation of propylene oxide with propylene glycol. The parenteral formulations can be presented in unit-dose or multi-dose sealed containers, such as ampoules or vials, and can be stored in a freeze-dried (lyophilized) condition requiring only the addition of a sterile liquid excipient, for example, water for injections, immediately prior to use. Extemporaneous injection solutions and suspensions can be prepared from sterile powders, granules, and tablets of the kind previously described.

In certain embodiments, injectable formulations are provided herein. The requirements for effective pharmaceutical carriers for injectable compositions are well-known to those of ordinary skill in the art (see, e.g., Pharmaceutics and Pharmacy Practice, J. B. Lippincott Company, Philadelphia, PA, Banker and Chalmers, eds., pages 238-250 (1982), and ASHP Handbook on Injectable Drugs, Toissel, 4th ed, pages 622-630 (1986)).

In some embodiments, topical formulations are provided herein. Topical formulations, including those that are useful for transdermal drug release, are suitable in the context of certain embodiments provided herein for application to skin. In some embodiments, the therapeutic agent alone or in combination with other suitable components, can be made into aerosol formulations to be administered via inhalation. These aerosol formulations can be placed into pressurized acceptable propellants, such as dichlorodifluoromethane, propane, nitrogen, and the like. They also may be formulated as pharmaceuticals for non-pressured preparations, such as in a nebulizer or an atomizer. Such spray formulations also may be used to spray mucosa.

In certain embodiments, the therapeutic agents provided herein can be formulated as inclusion complexes, such as cyclodextrin inclusion complexes, or liposomes. Liposomes can serve to target the therapeutic agents to a particular tissue. Liposomes also can be used to increase the half-life of the therapeutic agents. Many methods are available for preparing liposomes, as described in, for example, Szoka el al., Ann. Rev. Biophys. Bioeng., 9, 467 (1980) and U.S. Pat. Nos. 4,235,871, 4,501,728, 4,837,028, and 5,019,369.

In some embodiments, the therapeutic agents provided herein are formulated in time-released, delayed release, or sustained release delivery systems such that the delivery of the composition occurs prior to, and with sufficient time to, cause sensitization of the site to be treated. Such systems can avoid repeated administrations of the therapeutic agent, thereby increasing convenience to the subject and the physician, and may be particularly suitable for certain composition embodiments provided herein. In one embodiment, the compositions of the invention are formulated such that they are suitable for extended-release of the circRNA contained therein. Such extended-release compositions may be conveniently administered to a subject at extended dosing intervals. For example, in one embodiment, the compositions of the present invention are administered to a subject twice a day, daily or every other day. In an embodiment, the compositions of the present invention are administered to a subject twice a week, once a week, every ten days, every two weeks, every three weeks, every four weeks, once a month, every six weeks, every eight weeks, every three months, every four months, every six months, every eight months, every nine months or annually.

In some embodiments, a protein encoded by an inventive polynucleotide is produced by a target cell for sustained amounts of time. For example, the protein may be produced for more than one hour, more than four, more than six, more than 12, more than 24, more than 48 hours, or more than 72 hours after administration. In some embodiments the polypeptide is expressed at a peak level about six hours after administration. In some embodiments the expression of the polypeptide is sustained at least at a therapeutic level. In some embodiments, the polypeptide is expressed at least at a therapeutic level for more than one, more than four, more than six, more than 12, more than 24, more than 48, or more than 72 hours after administration. In some embodiments, the polypeptide is detectable at a therapeutic level in patient tissue (e.g., liver or lung). In some embodiments, the level of detectable polypeptide is from continuous expression from the circRNA composition over periods of time of more than one, more than four, more than six, more than 12, more than 24, more than 48, or more than 72 hours after administration.

In certain embodiments, a protein encoded by an inventive polynucleotide is produced at levels above normal physiological levels. The level of protein may be increased as compared to a control. In some embodiments, the control is the baseline physiological level of the polypeptide in a normal individual or in a population of normal individuals. In other embodiments, the control is the baseline physiological level of the polypeptide in an individual having a deficiency in the relevant protein or polypeptide or in a population of individuals having a deficiency in the relevant protein or polypeptide. In some embodiments, the control can be the normal level of the relevant protein or polypeptide in the individual to whom the composition is administered. In other embodiments, the control is the expression level of the polypeptide upon other therapeutic intervention, e.g., upon direct injection of the corresponding polypeptide, at one or more comparable time points.

In certain embodiments, the levels of a protein encoded by an inventive polynucleotide are detectable at 3 days, 4 days, 5 days, or 1 week or more after administration. Increased levels of protein may be observed in a tissue (e.g., liver or lung).

In some embodiments, the method yields a sustained circulation half-life of a protein encoded by an inventive polynucleotide. For example, the protein may be detected for hours or days longer than the half-life observed via subcutaneous injection of the protein or mRNA encoding the protein. In some embodiments, the half-life of the protein is 1 day, 2 days, 3 days, 4 days, 5 days, or 1 week or more.

Many types of release delivery systems are available and known to those of ordinary skill in the art. They include polymer based systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are lipids including sterols such as cholesterol, cholesterol esters, and fatty acids or neutral fats such as mono-di- and tri-glycerides; hydrogel release systems; sylastic systems; peptide based systems: wax coatings; compressed tablets using conventional binders and excipients; partially fused implants; and the like. Specific examples include, but are not limited to: (a) erosional systems in which the active composition is contained in a form within a matrix such as those described in U.S. Pat. Nos. 4,452,775, 4,667,014, 4,748,034, and 5,239,660 and (b) diffusional systems in which an active component permeates at a controlled rate from a polymer such as described in U.S. Pat. Nos. 3,832,253 and 3,854,480. In addition, pump-based hardware delivery systems can be used, some of which are adapted for implantation.

In some embodiments, the therapeutic agent can be conjugated either directly or indirectly through a linking moiety to a targeting moiety. Methods for conjugating therapeutic agents to targeting moieties is known in the art. See, for instance, Wadwa et al., J, Drug Targeting 3:111 (1995) and U.S. Pat. No. 5,087,616.

In some embodiments, the therapeutic agents provided herein are formulated into a depot form, such that the manner in which the therapeutic agent is released into the body to which it is administered is controlled with respect to time and location within the body (see, for example, U.S. Pat. No. 4,450,150). Depot forms of therapeutic agents can be, for example, an implantable composition comprising the therapeutic agents and a porous or non-porous material, such as a polymer, wherein the therapeutic agents are encapsulated by or diffused throughout the material and/or degradation of the non-porous material. The depot is then implanted into the desired location within the body and the therapeutic agents are released from the implant at a predetermined rate.

### Therapeutic Methods

In certain aspects, provided herein is a method of treating and/or preventing a condition, e.g., a viral infection.

In certain embodiments, the therapeutic agents provided herein are co-administered with one or more additional therapeutic agents (e.g., in the same pharmaceutical composition or in separate pharmaceutical compositions). In some embodiments, the therapeutic agent provided herein can be administered first and the one or more additional therapeutic agents can be administered second, or vice versa. Alternatively, the therapeutic agent provided herein and the one or more additional therapeutic agents can be administered simultaneously.

In some embodiments, the subject is a mammal. In some embodiments, the mammal referred to herein can be any mammal, including, but not limited to, mammals of the order Rodentia, such as mice and hamsters, or mammals of the order Logomorpha, such as rabbits. The mammals may be from the order Carnivora, including Felines (cats) and Canines (dogs). The mammals may be from the order Artiodactyla, including Bovines (cows) and Swines (pigs), or of the order Perssodactyla, including Equines (horses). The mammals may be of the order Primates, Ceboids, or Simoids (monkeys) or of the order Anthropoids (humans and apes). Preferably, the mammal is a human.

### Sequences

TABLE 1
IRES sequences.

| SEQ ID | | |
|---|---|---|
| NO: | IRES | Sequence |
| 1 | EMCV-A | cccccctctccctccccccctaacgttactggccgaagccgcttggaataaggccggt |
| | | gtgcgtttgtctatatgttattttccaccatattgccgtcttttggcaatgtgagggc |
| | | ccggaaacctggccctgtcttcttgacgagcattcctaggggtctttcccctctcgcc |
| | | aaaggaatgcaaggtctgttgaatgtcgtgaaggaagcagttcctctggaagcttctt |
| | | gaagacaaacaacgtctgtagcgaccctttgcaggcagcggaaccccccacctggcga |
| | | caggtgcctctgcggccaaaagccacgtgtataagatacacctgcaaaggcggcacaa |
| | | ccccagtgccacgttgtgagttggatagttgtggaaagagtcaaatggctctcctcaa |
| | | gcgtattcaacaaggggctgaaggatgcccagaaggtaccccattgtatgggatctga |
| | | tctggggcctcggtgcacatgctttacatgtgtttagtcgaggttaaaaaacgtctag |
| | | gccccccgaaccacggggacgtggttttcctttgaaaaacacgatgataatatggcca |
| | | caacc |
| 2 | EMCV-B | ctccccctccccccccttactatactggccgaagccacttggaataaggccggtgtgc |
| | | gtttgtctacatgctattttctaccgcattaccgtcttatggtaatgtgagggtccag |
| | | aacctgaccctgtcttcttgacgaacactcctaggggtctttcccctctcgacaaagg |
| | | agtgtaaggtctgttgaatgtcgtgaaggaagcagttcctctggaagcttcttaaaga |
| | | caaacaacgtctgtagcgaccctttgcaggcagcggaaccccccacctggtgacaggt |
| | | gcctctgcggccaaaagccacgtgtataagatacacctgcaaaggcggcacaacccca |
| | | gtgccacgttgtgagttggatagttgtggaaagagtcaaatggctctcctcaagcgta |
| | | ttcaacaaggggctgaaggatgcccagaaggtaccccattgtatgggatctgatctgg |
| | | ggcctcggtgcacgtgctttacacgtgttgagtcgaggtgaaaaaacgtctaggcccc |
| | | ccgaaccacggggacgtggttttcctttgaaaaccacgattacaat |
| 3 | EMCV-Bf | ttgccagtctgctcgatatcgcaggctgggtccgtgactacccactccccctttcaac |
| | | gtgaaggctacgatagtgccagggcgggtactgccgtaagtgccaccccaaacaacaa |
| | | caacaaaacaaactccccctccccccccttactatactggccgaagccacttggaata |
| | | aggccggtgtgcgtttgtctacatgctattttctaccgcattaccgtcttatggtaat |
| | | gtgagggtccagaacctgaccctgtcttcttgagaacactcctaggggtctttcccct |
| | | ctcgacaaaggagtgtaaggtctgttgaatgtcgtgaaggaagcagttcctctggaag |
| | | cttcttaaagacaaacaacgtctgtagcgaccctttgcaggcagcggaaccccccacc |
| | | tggtgacaggtgcctctgcggccaaaagccacgtgtataagatacacctgcaaaggcg |
| | | gcacaaccccagtgccacgttgtgagttggatagttgtggaaagagtcaaatggctct |
| | | cctcaagcgtattcaacaaggggctgaaggatgcccagaaggtaccccattgtatggg |
| | | atctgatctggggcctcggtgcacgtgctttacacgtgttgagtcgaggtgaaaaaac |
| | | gtctaggccccccgaaccacggggacgtggttttcctttgaaaaccacgattacaat |
| 4 | EMCV-Cf | ttgccagtctgctcgatatcgcaggctgggtccgtgactacccactccccctttcaac |
| | | gtgaaggctacgatagtgccaggggggtactgccgtaagtgccaccccaaaacaacaa |
| | | caaccccccctctccctccTccccccctaacgttactggccgaagccgcttggaataa |
| | | ggccggtgtgcgtttgtctatatgttattttccaccatattgccgtcttttggcaatg |
| | | tgagggcccggaaacctggccctgtcttcttgacgagcattcctaggggtctttcccc |
| | | tctcgccaaaggaatgcaaggtctgttgaatgtcgtgaaggaagcagttcctctggaa |
| | | gcttcttgaagacaaacaacgtctgtagcgaccctttgcaggcagcggaaccccccac |
| | | ctggcgacaggtgcctctgcggccaaaagccacgtgtataagatacacctgcaaaggc |
| | | ggcacaaccccagtgccacgttgtgagttggatagttgtggaaagagtcaaatggctc |
| | | tcctcaagcgtattcaacaaggggctgaaggatgcccagaaggtaccccattgtatgg |
| | | gatctgatctggggcctcggtgcacatgctttacatgtgtttagtcgaggttaaaaaa |
| | | cgtctaggccccccgaaccacggggacgtggttttcctttgaaaaacacgatgataat |
| 5 | EMCV pEC9 | ccccccccctaacgttactggccgaagccgcttggaataaggccggtgtgcgtttgtc |
| | | tatatgttattttccaccatattgccgtcttttggcaatgtgagggcccggaaacctg |
| | | gccctgtcttcttgacgagcattcctaggggtctttcccctctcgccaaaggaatgca |
| | | aggtctgttgaatgtcgtgaaggaagcagttcctctggaagcttcttgaagacaaaca |
| | | acgtctgtagcgaccctttgcaggcagcggaaccccccacctggcgacaggtgcctct |
| | | gcggccaaaagccacgtgtataagatacacctgcaaaggcggcacaaccccagtgcca |
| | | cgttgtgagttggatagttgtggaaagagtcaaatggctctcctcaagcgtattcaac |
| | | aaggggctgaaggatgcccagaaggtaccccattgtatgggatctgatctggggcctc |
| | | ggtgcacatgctttacatgtgtttagtcgaggttaaaaaacgtctaggccccccgaac |
| | | cacggggacgtggttttcctttgaaaaacacgatgataat |
| 6 | Picobirnavirus | gtaaattaaatgctatttacaaaatttaaacagaaaggagagatgttatgaaccggtt |
| | | ttacaaggtttcatacatcgaaaatagcactacctggggcagccgacacactaacatc |
| | | gtctgtttaaccagaagtgttactgaaaggaggttattta |
| 7 | HCV QC64 | acctgcccctaataggggcgacactccgccatgaatcactcccctgtgaggaactact |
| | | gtcttcacgcagaaagcgtctagccatggcgttagtatgagtgtcgtacagcctccag |
| | | gcccccccctcccgggagagccatagtggtctgcggaaccggtgagtacaccggaatt |
| | | gccgggaagactgggtcctttcttggataaacccactctatgcccggacatttgggcg |
| | | tgcccccgcaagactgctagccgagtagcgttgggttgcgaaaggccttgtggtactg |
| | | cctgatagggtgcttgcgagtgccccgggaggtctcgtagaccgtgcatc |
| 8 | Human | ctacaagctttgtgtaaacaaacttttgtttggcttttctcaagcttctctcacatca |
| | Cosavirus | ggccccaaagatgtcctgaaggtaccccgtgtatctgaggatgagcaccatcgactac |
| | E/D | ccggacctgcaaaattttgcaaacgcatgtggtatcccagccccctcctctcggggag |
| | | ggggctttgctcactcagcacaggatctgatcaggagatccacctccggtgctttaca |
| | | ccggggcgtggatttaaaaattgcccaaggcctggcgcacaacctaggggactaggtt |
| | | ttccttatattttaaagctgtcaat |
| 9 | Human | gtcttaggacgacgcatgtggtatcccagcccccgcctacattggcgggggcttttga |
| | Cosavirus F | agcaccagacactggatctgatcaggaggagggtagctgctttacagcccctcttaaa |
| | | aattgcccaaggtccggccacccaacctaggggactaggttttccttttatttttaaa |
| | | ttgtcatt |
| 10 | Human | acatgggggagactgcatgtggcagtcttgaaacgtgtggtttgacgtctaccttata |
| | Cosavirus | tggcagtgggtggagtactgcaaagatgtcaccgtgctttacacggtttttgaacccc |
| | JMY | acaccggctgtttgacgctcgtagggcagcaggtttattttcattaaaattcttactt |
| | | tctagctgcatgagttctattcatgcagacggagtgatactcccgttccttcttggac |
| | | aggttgcctccacgccctttgtggatcttaaggtgaccaagtcactggtgttggaggt |
| | | gaagatagagagtcctcttgggaatgtcatgtggctgtgccaggggttgtagcgatgc |
| | | cattcgtgtgtgcggatttcctctcgtggtgacacgagcctcacaggccaaaagcccc |
| | | gtccgaaaggacccgaatggtggagtgaccctgactcccccctgcatagttttgtgat |
| | | taggaacttgaggaatttctgtcataaatctctatcacatcaggccccaaagatgtcc |
| | | tgaaggtaccctgtgtatctgaggatgagcaccaccgactacccggacttgcattagc |
| | | agacacatgtggttgcccagccccacctcttcagaggtggggctttgctcactcagca |
| | | caggatctgatcaggagccccgctcgtgtgctttacactcgacgcggggttaaaaatt |
| | | gcccaaggcctggcacaacaacctaggggactaggttttcctatttttgtaaattatg |
| | | tcaat |
| 11 | Rhinovirus | gtgacaatcagccagattgttaacggtcaagcacttctgtttccccggtacccttgta |
| | NAT001 | tacgcttcacccgaggcgaaaagtgaggttatcgttatccgcaaagtgcctacgagaa |
| | | gcctagtagcacttttgaagcctatggctggtcgctcaactgtttacccagcagtaga |
| | | cctggcagatgaggctagatgttccccaccagcgatggtgatctagcctgcgtggctg |
| | | cctgcacactctattgagtgtgaagccagaaagtggacaaggtgtgaagagcctattg |
| | | tgctcactttgagtcctccggcccctgaatgtggctaatcctaaccccgtagctgttg |
| | | catgtaatccaacatgtctgcagtcgtaatgggcaactatgggatggaaccaactact |
| | | ttgggtgtccgtgtttcttgtttttctttatgcttgcttatggtgacaactgtagtta |
| | | ttacatttgttacc |
| 12 | HRV14 | ttaaaacagcggatgggtatcccaccattcgacccattgggtgtagtactctggtact |
| | | atgtacctttgtacgcctgtttctccccaaccacccttccttaaaattcccacccatg |
| | | aaacgttagaagcttgacattaaagtacaataggtggcgccatatccaatggtgtcta |
| | | tgtacaagcacttctgtttcccaggagcgaggtataggctgtacccactgccaaaagc |
| | | ctttaaccgttatccgccaaccaactacgtaacagttagtaccatcttgttcttgact |
| | | ggacgttcgatcaggtggattttccctccactagtttggtcgatgaggctaggaattc |
| | | cccacgggtgaccgtgtcctagcctgcgtggcggccaacccagcttatgctgggacgc |
| | | ccttttaaggacatggtgtgaagactcgcatgtgcttggttgtgagtcctccggcccc |
| | | tgaatgcggctaaccttaaccctagagccttatgccacgatccagtggttgtaaggtc |
| | | gtaatgagcaattccgggacgggaccgactactttgggtgtccgtgtttctcattttt |
| | | cttcatattgtcttatggtcacagcatatatatacatatactgtgatc |
| 13 | HRV89 | ttaaaactgggagtgggttgttcccactcactccacccatgcggtgttgtactctgtt |
| | | attacggtaactttgtacgccagtttttcccacccttccccataatgtaacttagaag |
| | | tttgtacaatatgaccaataggtgacaatcatccagactgtcaaaggtcaagcacttc |
| | | tgtttccccggtcaatgaggatatgctttacccaaggcaaaaaccttagagatcgtta |
| | | tccccacactgcctacacagagcccagtaccatttttgatataattgggttggtcgct |
| | | ccctgcaaacccagcagtagacctggcagatgaggctggacattccccactggcgaca |
| | | gtggtccagcctgcgtggctgcctgctcacccttcttgggtgagaagcctaattattg |
| | | acaaggtgtgaagagccgcgtgtgctcagtgtgcttcctccggcccctgaatgtggct |
| | | aaccttaaccctgcagccgttgcccataatccaatgggtttgcggtcgtaatgcgtaa |
| | | gtgcgggatgggaccaactactttgggtgtccgtgtttcctgtttttcttttgattgc |
| | | attttatggtgacaatttatagtgtatagattgtcatc |
| 14 | HRVC-02 | ttaaaactgggtacaggttgttcccacctgtatcacccacgtggtgtggtgctcttgt |
| | | attccggtacacttgcacgccagtttgccacccctcacccgtcgtaacttagaagcta |
| | | acaactcgaccaacaggcggtggtaaaccataccacttacggtcaagcactcctgttt |
| | | ccccggtatgcgaggaatagactcctacagggttgaagcctcaagtatcgttatccgc |
| | | attggtactacgcaaagcttagtagtgccttgaaagtcccttggttggtcgctccgct |
| | | agtttcccctagtagacctggcagatgaggcaggacactccccactggcgacagtggt |
| | | cctgcctgcgtggctgcctgcgcacccttaggggtgcgaagccaagtgacagacaagg |
| | | tgtgaagagccccgtgtgctaccaatgagtcctccggcccctgaatgcggctaatcca |
| | | accccacagctattgcacacaagccagtgtgtatgtagtcgtaatgagcaattgtggg |
| | | acggaaccgactactttgggtgtccgtgtttccttttattcttatcattctgcttatg |
| | | gtgacaatactgtgaaatagtgttgttacc |
| 15 | HRV-A21 | taaaactggatccaggttgttcccacctggatctcctattgggagttgtactctatta |
| | | ttccggtaattttgtacgccagttttatcttccccctccccaattgtaacttagaagg |
| | | ttatcaatacgaccaataggtggtagttagccaaactaccaaaggtcaagcacttctg |
| | | tttccccggtcaaagttgatatgctccaacagggcaaaaacaactgagatcgttatcc |
| | | gcaaagtgcctacgcaaagcctagtaacacctttgaagatttatggttggtcgttccg |
| | | ctatttcccatagtagacctggcagatgaggctagaaatcccccactggcgacagtgc |
| | | tctagcctgcgtggctgcctgcgcaccccttgggtgcgaagccatacattggacaagg |
| | | tgtgaagagccccgtgtgctcactttgagtcctccggcccctgaatgtggctaaccct |
| | | taaccctgcagctagtgcatgtaatccaacatgttgctagtcgtaatgagtaattgcg |
| | | ggacgggaccaactactttgggtgtccgtgtttcactttttccttttaatattgctta |
| | | tggtgacaatatatatagctatatatattgacacc |
| 16 | Salivirus A | ttcccctgcaaccattacgcttactcgcatgtgcattgagtggtgcatgtgttgaaca |
| | SH1 | aacagctacactcacatggggggggttttcccgccctacggcttctcgcgaggcccac |
| | | ccctcccctttctcccataactacagtgctttggtaggtaagcatcctgatcccccgc |
| | | ggaagctgctcacgtggcaactgtggggacccagacaggttatcaaaggcacccggtc |
| | | tttccgccttcaggagtatccctgctagcgaattctagtagggctctgcttggtgcca |
| | | acctcccccaaatgcgcgctgcgggagtgctcttccccaactcaccctagtatcctct |
| | | catgtgtgtgcttggtcagcatatctgagacgatgttccgctgtcccagaccagtcca |
| | | gtaatggacgggccagtgtgcgtagtcgtcttccggcttgtccggcgcatgtttggtg |
| | | aaccggtggggtaaggttggtgtgcccaacgcccgtactcaggggatacctcaaggca |
| | | cccaggaatgccagggaggtaccccgcttcacagcgggatctgaccctggggtaaatg |
| | | tctgcggggggtcttcttggcccacttctcagtacttttcagg |
| 17 | Salivirus FHB | acatggggggtctgcggacggcttcggcccacccgcgacaagaatgccgtcatctgtc |
| | | ctcattacccgtattccttcccttcccccgcaaccaccacgcttactcgcgcacgtgt |
| | | tgagtggcacgtgcgttgtccaaacagctacacccacacccttcggggcgggtttgtc |
| | | ccgccctcgggttcctcgcggaacccccccctccctctctctctttctatccgccctc |
| | | acttcccataactacagtgctttggtaggtgagcaccctgaccccccgcggaagctgc |
| | | taacgtggcaactgtggggatccaggcaggttatcaaaggcacccggtctttccgcct |
| | | tcaggagtatctctgccggtgaattccggtagggctctgcttggtgccaacctccccc |
| | | aaatgcgcgctgcgggagtgctcttccccaactcatcttagtaacctctcatgtgtgt |
| | | gcttggtcagcatatctgaggcgacgttccgctgtcccagaccagtccagcaatggac |
| | | gggccagtgtgcgtagtcgctttccggttttccggcgcatgtttggcgaaacgctgag |
| | | gtaaggttggtgtgcccaacgcccgtaatttggtgatacctcaagaccacccaggaat |
| | | gccagggaggtaccccacttcggtgggatctgaccctgggctaattgtctacggtggt |
| | | tcttcttgcttccacttctcttttttctggcatg |
| 18 | Salivirus | tatggcagggggcttgtggacggcttcggcccacccacagcaagaatgccatcatctg |
| | NG-J1 | tcctcacccccaattttcccttttcttcccctgcaaccattacgcttactcgcatgtg |
| | | cattgagtggtgcatgtgttgaacaaacagctacactcacatggggggggttttcccg |
| | | ccctacggcctctcgcgaggcccaccccttccctccccttataactacagtgctttgg |
| | | taggtaagcatcctgatcccccgcggaagctgctcacgtggcaactgtggggacccag |
| | | acaggttatcaaaggcacccggtctttccgccttcaggagtatccctactagtgaatt |
| | | ctagcggggctctgcttggtgccaacctcccccaaatgcgcgctgcgggagtgctctt |
| | | ccccaactcaccctagtatcctctcatgtgtgtgcttggtcagcatatctgagacgat |
| | | gttccgctgtcccagaccagtccagtaatggacgggccagtgcgtgtagtcgtcttcc |
| | | ggcttgtccggggcatgtttggtgaaccggtggggtaaggttggtgtgcccaacgccc |
| | | gtactttggtgacacctcaagaccacccaggaatgccagggaggtaccccacctcacg |
| | | gtgggatctgaccctgggctaattgtctacggtggttcttcttgcttccacttctttc |
| | | ttctgttcacg |
| 19 | Human | tttgaaaggggtctcctagagagcttggccgtcgggccttataccccgacttgctgag |
| | Parechovirus | tttctctaggagagcccttttcccagccctgaggcggctggtcaataaaagcctcaaa |
| | 1 | cgtaactaacacctaagaagatcatgtaaaccctatgcctggtctccactattcgaag |
| | | gcaacttgcaataagaagagtgggatcaagacgcttaaagcatagagacagttttttt |
| | | tctaacccacatttgtgtggggggcagatggcgtgccataactctaatagtgagatac |
| | | cacgcttgtggaccttatgctcacacagccatcctctagtaagtttgtgagacgtctg |
| | | gtgacgtgtgggaacttattggaaacaacattttgctgcaaagcatcctactgccagc |
| | | ggaaaaacacctggtaacaggtgcctctggggccaaaagccaaggtttaacagaccct |
| | | ttaggattggttctaaacctgagatgttgtggaagatatttagtacctgctgatctgg |
| | | tagttatgcaaacactagttgtaaggcccatgaaggatgcccagaaggtacccgtagg |
| | | taacaagtgacactatggatctgatttggggccagatacctctatcttggtgatctgg |
| | | ttaaaaaacatctaatgggccaaacccgggggggatccccggtttcctcttattctat |
| | | caatgccact |
| 20 | Crohivirus B | gtataagagacaggtgtttgccttgtcttcggactggcatcttgggaccaacccccct |
| | | tttccccagccatgggttaaatggcaataaaggacgtaacaactttgtaaccattaag |
| | | ctttgtaattttgtaaccactaagctttgtgcacataatgtaaccatcaagcttgtta |
| | | gtcccagcaggaggtttgcatgcttgtagccgaaatggggctcgaccccccatagtag |
| | | gatacttgattttgcattccattgtggacctgcaaactctacacatagaggctttgtc |
| | | ttgcatctaaacacctgagtacagtgtgtacctagaccctatagtacgggaggaccgt |
| | | ttgtttcctcaataaccctacataataggctaggtgggcatgcccaatttgcaagatc |
| | | ccagactgggggtcggtctgggcagggttagatccctgttagctactgcctgataggg |
| | | tggtgctcaaccatgtgtagtttaaattgagctgttcatatacc |
| 21 | Yc-3 | actgaagatcctacagtaactactgccccaatgaacgccacagatgggtctgctgatg |
| | | actacctatcttagtgctagttgaggtttgaagtgagccggtttttagaagaaccagt |
| | | ttctgaacattatcatccccagcatctattctatacgcacaagatagatagtcatcag |
| | | cagacacatctgtgctactgcttgatagagttgcggctggtcaacttagattggtata |
| | | accagttgagtggcaa |
| 22 | Rosavirus | tatgcatcactggacggcctaacctcggtcgtggcttcttgccgatttcagcgctacc |
| | M-7 | aggctttctggtctcgccaggcgttgattagtaggtgcactgtctaagtgaagacagc |
| | | agtgctctctgtgaaaagttgatgacactcttcaggtttgtagcgatcactcaaggct |
| | | agcggatttccccgtgtggtaacacacgcctctaggcccagaaggcacggtgttgaca |
| | | gcaccccttgagtggctggtcttccccaccagcacctgatttgtggattcttcctagt |
| | | aacggacaagcatggctgctcttaagcattcagtgcgtccggggctgaaggatgccca |
| | | gaaggtacccgcaggtaacgataagctcactgtggatctgatctggggctgcgggctg |
| | | ggtgtctttccacccagccaaaacccgtaaaacggtagtcgcagttaaaaaacgtcta |
| | | ggccccacccccccagggatggggggttcccttaaaccctcacaagttcaac |
| 23 | Shanbavirus | tgaaaagggggcgcagggtggtggtggttactaaatacccaccatcgccctgcacttc |
| | A | ccttttcccctgtggctcagggtcacttagccccctctttgggttaccagtagttttc |
| | | tacccctgggcacagggttaactatgcaagacggaacaacaatctcttagtccccctc |
| | | gccgatagtgggctcgacccccatgtgtaggagtggataagggacggagtgagccgat |
| | | acggggaagagtgtgcggtcacaccttaattccatgagcgctgcgaagaaggaagctg |
| | | tgaacaatggcgacctgaaccgtacacatggagctccacaggcatggtactcgttaga |
| | | ctacgcagcctggttgggagtgggtataccctgggtgagccgccagtgaatgggagtt |
| | | cactggttaacacacactgcctgatagggtcagggcctcctgtccccgccgtaatgag |
| | | gtagaccatatgcc |
| 24 | Pasivirus A | gcggctggatattctggccgtgcaactgcttttgaccagtggctctgggtaacttagc |
| | | caaagtgtccttctccctttccctattatatgttttatggctttgtctggtcttgttt |
| | | agtttatatataagatcctttccgccgatatagacctcgacagtctagtgtaggagga |
| | | ttggtgatattaatttgccccagaagagtgaccgtgacacatagaaaccatgagtaca |
| | | tgtgtatccgtggaggatcgcccgggactggattccatatcccattgccatcccaaca |
| | | agcggagggtatacccactatgtgcacgtctgcagtgggagtctgcagatttagtcat |
| | | actgcctgatagggtgtgggcctgcactctggggtactcaggctgtttatataat |
| 25 | Pasivirus A 2 | gctggactttctggctgcgcaactgcttttaaccagtggctctgggttacttagccaa |
| | | aaccccctttccccgtaccctagtttgtgtgtgtattattattttgttgttgttttgt |
| | | aaatttttatataagatcctttccgccgatatagacctcgacagtctagtgtaggagg |
| | | attggtgatattaatatgccccagaagagtgaccgtgacacatagaaaccatgagtac |
| | | atgtgtatccgtggaggatcgcccgggactggattccatatcccattgccatcccaac |
| | | aaacggagggtatacccgctatgtgcgcgtctacagtgggaatctgtagatttagtca |
| | | tactgcctgatagggtgtgggcctgcactctggggtactcaggctgtttatataat |
| 26 | Echovirus | ttaaaacagcctgtgggttgttcccatccacagggcccactgggcgccagcactctgg |
| | E14 | tattgcggtaccttagtgcgcctgttttatatacccgtcccccaaacgtaacttagac |
| | | gcatgtcaacgaagaccaatagtaagcgcagcacaccagctgtgttccggtcaagcac |
| | | ttctgttaccccggaccgagtatcaataagctactcacgtggctgaaggagaaaacgt |
| | | tcgttacccgaccaattacttcaagaaacctagtaacaccatgaaggttgcgcagtgt |
| | | ttcgctccgcacaaccccagtgtagatcaggtcgatgagtcaccgcattccccacggg |
| | | tgaccgtggcggtggctgcgctggcggcctgcccatggggaaacccatgggacgcttc |
| | | aatactgacatggtgcgaagagtctattgagctaattggtagtcctccggcccctgaa |
| | | tgcggctaatcctaactgcggagcagatacccacacaccagtgggcagtctgtcgtaa |
| | | cgggcaactctgcagcggaaccgactactttgggtgtccgtgtttctctttatcctta |
| | | tactggctgcttatggtgacaattgagagattgttaccatatagctattggattggcc |
| | | atccggtgacaaatagagcaattgtgtatttgtttgttggtttcgtgccattaaatta |
| | | caaggttctaaacacccttaatcttattatagcattcaacacaacaaa |
| 27 | Human | gtacattagatgcgtcatctgcaactttagtcaataaattacctccaatgtcattacc |
| | Parechovirus | aacattccctaccttttcactaacacctaagacaacaagtacctatgcctggtctcca |
| | 5 | ctattcgaaggcaacttgcaataagaagagtggaattaagacgcttaaagcatagagc |
| | | tagttatcttttctaacccacaaagttttgtggggggcagatggcgtgccataactct |
| | | attagtgagataccatgcttgtggatcttatgctcacacagccatcctctagtaagtt |
| | | gataaggtgtctggtgatatgtgggaactcacatgaaccattaatttaccgtaaggta |
| | | tcctatagccagcggaatcacatctggtgacagatgcctctggggccgaaagccaagg |
| | | tttaacagaccctataggattggtttcaaaacctgaattgatgtggattgtgtatagt |
| | | acctgttgatctggtaacagtgtcaacactagttgtaaggcccacgaaggatgcccag |
| | | aaggtacccgtaggtaacaagtgacactatggatctgatctggggccagctacctcta |
| | | tcatggtgagttggttaaaaaacgtctagtgggccaaacccaggggggatccctggtt |
| | | tccttttacctaatcaaagccact |
| 28 | Aichi Virus | tttgaaaaggggggggggggcctcggccccctcaccctcttttccggtggtctggtcc |
| | | cggaccaccgttactccattcagcttcttcggaacctgttcggaggaattaaacgggc |
| | | acccatactccccccaccccccttttgtaactaagtatgtgtgctcgtgatcttgact |
| | | cccacggaacggaccgatccgttggtgaacaaacagctaggtccacatcctcccttcc |
| | | cctgggagggcccccgccctcccacatcctccccccagcctgacgtatcacaggctgt |
| | | gtgaagcccccgcgaaagctgctcacgtggcaattgtgggtccccccttcatcaagac |
| | | accaggtctttcctccttaaggctagccccggcgtgtgaattcacgttgggcaactag |
| | | tggtgtcactgtgcgctcccaatctcggccgcggagtgctgttccccaagccaaaccc |
| | | ctggcccttcactatgtgcctggcaagcatatctgagaaggtgttccgctgtggctgc |
| | | caacctggtgacaggtgccccagtgtgcgtaaccttcttccgtctccggacggtagtg |
| | | attggttaagatttggtgtaaggttcatgtgccaacgccctgtgcgggatgaaacctc |
| | | tactgccctaggaatgccaggcaggtaccccacctccggggggatctgagcctgggct |
| | | aattgtctacgggtagtttcatttccaatccttttatgtcggagtc |
| 29 | Hepatitis A | ttcaagaggggtctccggagttttccggaacccctcttggaagtccatggtgagggga |
| | Virus HA16 | cttgatacctcaccgccgtttgcctaggctataggctaaatttccctttccctgtcct |
| | | tcccctatttccttttgttttgtttgtaaatattaattcctgcaggttcagggttctt |
| | | taatctgtttctctataagaacactcaatttttcacgctttctgtctcctttcttcca |
| | | gggctctccccttgccctaggctctggccgttgcgcccggcggggtcaactccatgat |
| | | tagcatggagctgtaggagtctaaattggggacgcagatgtttgggacgtcgccttgc |
| | | agtgttaacttggctttcatgaacctctttgatcttccacaaggggtaggctacgggt |
| | | gaaacctcttaggctaatacttcaatgaagagatgccttggatagggtaacagcggcg |
| | | gatattggtgagttgttaagacaaaaaccattcaacgccggaggactggctctcatcc |
| | | agtggatgcattgagggaattgattgtcagggctgtctctaggtttaatctcagacct |
| | | ctctgtgcttagggcaaacactatttggccttaaatgggatcctgtgagagggggtcc |
| | | ctccattgacagctggactgttctttggggccttatgtggtgtttgcctctgaggtac |
| | | tcaggggcatttaggtttttcctcattcttaaataata |
| 30 | Phopivirus | gggagtaaacctcaccaccgtttgccgtggtttacggctacctatttttggatgtaaa |
| | | tattaattcctgcaggttcaggtctcttgaattatgtccacgctagtggcactctctt |
| | | acccataagtgacgccttagcggaacctttctacacttgatgtggttaggggttacat |
| | | tatttccctgggccttctttggccctttttcccctgcactatcattctttcttccggg |
| | | ctctcagcatgccaatgttccgaccggtgcgcccgccggggttaactccatggttagc |
| | | atggagctgtaggccctaaaagtgctgacactggaactggactattgaagcatacact |
| | | gttaactgaaacatgtaactccaatcgatcttctacaaggggtaggctacgggtgaaa |
| | | ccccttaggttaatactcatattgagagatacttctgataggttaaggttgctggata |
| | | atggtgagtttaacgacaaaaaccattcaacagctgtgggccaacctcatcaggtaga |
| | | tgcttttggagccaagtgcgtaggggtgtgtgtggaaatgcttcagtggaaggtgccc |
| | | tcccgaaaggtcgtaggggtaatcaggggcagttaggtttccacaattacaatttgaa |
| 31 | CVA10 | gctcttccgatctgggttgttcccacccacagggcccactgggcgccagcactctgat |
| | | tccacggaatctttgtgcgcctgttttacaacccttcccaatttgtaacgtagaagca |
| | | atacacactactgatcaatagtaggcatggcgcgccagtcatgtcatgatcaagcact |
| | | tctgttcccccggactgagtatcaatagactgctcacgcggttgaaggagaaaacgtt |
| | | cgttacccggctaactacttcgagaaacctagtagcaccatggaagctgcggagtgtt |
| | | tcgctcagcactttccccgtgtagatcaggtcgatgagtcactgcaatccccacgggc |
| | | gaccgtggcagtggctgcgttggcggcctgcctatggggcaacccataggacgctcta |
| | | atgtggacatggtgcgaagagtctattgagctagttagtagtcctccggcccctgaat |
| | | gcggctaatcctaactgcggagcacatgccttcaacccaggaggtggtgtgtcgtaac |
| | | gggtaactctgcagcggaaccgactactttgggtgtccgtgtttccttttatccttat |
| | | attggctgcttatggtgacaatcacggaattgttgccatatagctattggattggcca |
| | | tccggtgtctaacagagctattgtatacctatttgttggatttactcccctatcatac |
| | | aaatctctgaacactttgtgctttatactgaacttaaacacacgaaa |
| 32 | Enterovirus C | ttaaaacagctctggggttgttcccaccccagaggcccacgtggcggccagtacaccg |
| | | gtaccacggtacccttgtacgcctgttttatactcccctccccgtaaactagaagcac |
| | | gaaacacaagttcaatagaagggggtacagaccagtaccaccacgaacaagcacttct |
| | | gttcccccggtgaggtcacatagactgtccccacggtcaaaagtgactgatccgttat |
| | | ccgctcacgtacttcggaaagcctagtaccaccttggaatctacgatgcgttgcgctc |
| | | agcactcgaccccggagtgtagcttaggctgatgagtctggacgttccccactggtga |
| | | cagtggtccaggctgcgttggcggcctacctgtggtccaaaaccacaggacgctagta |
| | | gtgaacaaggtgtgaagagcccactgagctacctgagaatcctccggcccctgaatgc |
| | | ggctaatcccaaccacggagcaggtaatcgcaaaccagcggtcagcctgtcgtaacgc |
| | | gtaagtctgtggcggaaccgactactttgggtgtccgtgtttccttttatttttatgg |
| | | tggctgcttatggtgacaatcatagattgttatcataaagcaaattggattggccatc |
| | | cggagtgagctaaactatctatttctctgagtgttggattcgtttcacccacattctg |
| | | aacaatcagcctcattagtgttaccctgttaataagacgatatcatcacg |
| 33 | Enterovirus D | ttaaaacagctctggggttgttcccaccccagaggcccacgtggcggctagtactccg |
| | | gtaccccggtacccttgtacgcctgttttatactccctttcccaagtaactttagaag |
| | | aaataaactaatgttcaacaggagggggtacaaaccagtaccaccacgaacacacact |
| | | tctgtttccccggtgaagttgcatagactgtacccacggttgaaagcgatgaatccgt |
| | | tacccgcttaggtacttcgagaagcctagtatcatcttggaatcttcgatgcgttgcg |
| | | atcagcactctaccccgagtgtagcttgggtcgatgagtctggacaccccacaccggc |
| | | gacgtggtccaggctgcgttggcggcctacccatggctagcaccatgggacgctagtt |
| | | gtgaacaaggtgcgaagagcctattgagctacctgagagtcctccggcccctgaatgc |
| | | ggctaatcccaaccacggagcaaatgctcacaatccagtgagtggtttgtcgtaatgc |
| | | gcaagtctgtggcggaaccgactactttgggtgtccgtgtttccttttatttttatta |
| | | tggctgcttatggtgacaatctgagattgttatcatatagctattggattagccatcc |
| | | ggtgatatcttgaaattttgccataactttttcacaaatcctacaacattacactaca |
| | | ctttctcttgaataattgagacaactcata |
| 34 | Enterovirus J | ttaaaatagcctcagggttgttcccaccctgagggcccacgtggtgtagtactctggt |
| | | attacggtacctttgtacgcctattttatacccccttccccaagtaatttagaagcaa |
| | | gcacaaaccagttcagtagtaagcagtacaatccagtactgtaatgaacaagtacttc |
| | | tgttaccccggaagggtctatcggtaagctgtacccacggctgaagaatgacctaccg |
| | | ttaaccggctacctacttcgagaagcctagtaatgccgttgaagttttattgacgtta |
| | | cgctcagcacactaccccgtgtgtagttttggctgatgagtcacggcactccccacgg |
| | | gcgaccgtggccgtggctgcgttggcggccaaccaaggagtgcaagctccttggacgt |
| | | catattacagacatggtgtgaagagcctattgagctaggtggtagtcctccggcccct |
| | | gaatgcggctaatcctaactccggagcatatcggtgcgaaccagcacttggtgtgttg |
| | | taatacgtaagtctggagcggaaccgactactttgggtgtccgtgtttcctgttttaa |
| | | cttttatggctgcttatggtgacaatttaacattgttaccatatagctgttgggttgg |
| | | ccatccggattttgttataaaaccatttcctcgtgccttgacctttaacacatttgtg |
| | | aacttctttaaatcccttttattagtccttaaatactaaga |
| 35 | Human | aactgttgttgtagcaatgcgcatattgctacttcggtacgcctaattggtaggcgcc |
| | Pegivirus 2 | cggccgaccggccccgcaagggcctagtaggacgtgtgacaatgccatgagggatcat |
| | | gacactggggtgagcggaggcagcaccgaagtcgggtgaactcgactcccagtgcgac |
| | | cacctggcttggtcgttcatggagggcatgcccacgggaacgctgatcgtgcaaaggg |
| | | atgggtccctgcactggtgccatgcgcggcaccactccgtacagcctgatagggtggc |
| | | ggcgggcccccccagtgtgacgtccgtggagcgcaac |
| 36 | GBV-C | tgacgtgggggggttgatTTTccccccccggcactgggtgcaagccccagaaaccgac |
| | GT110 | gcctatctaagtagacgcaatgactcggcgccgactcggcgaccggccaaaaggtggt |
| | | ggatgggtgatgacagggttggtaggtcgtaaatcccggtcatcctggtagccactat |
| | | aggtgggtcttaagagaaggtcaagattcctcttacgcctgcggcgagaccgcgcacg |
| | | gtccacaggtgttggccctaccggtgtgaataagggcccgacatcaggc |
| 37 | GBV-C | gacgtgggggggttgatccccccccTTTggcactgggtgcaagccccagaaaccgacg |
| | K1737 | cctatttaaacagacgttaagaaccggcgccgacccggcgaccggccaaaaggtggtg |
| | | gatgggtgatgccagggttggtaggtcgtaaatcccggtcatcttggtagccactata |
| | | ggtgggtcttaagggttggttaaggtccctctggcgcttgtggcgagaaagcgcacgg |
| | | tccacaggtgttggccctaccggtgtgaataagggcccgacgtcaggctcgtcgttaa |
| | | accgagcccactacccacctgggcaaacaacgcccacgtacggtccacgtcgcccttc |
| | | aatgtctctcttgaccaataggcttagccggcgagttgacaaggaccagtgggggctg |
| | | ggcggtaggggaaggacccctgccgctgcccttcccggtggagtgggaaatgc |
| 38 | GBV-C Iowa | tgacgtgggggggttgatccGccccccccggcactCggtgcaagccccataaaccgac |
| | | gcctatctaagtagacgcaatgactcggcgccgactcggcgaccggccaaaaggtggt |
| | | ggatgggtggtgacagggttggtaggtcgtaaatcccggtcatcctggtagccactat |
| | | aggtgggtcttaagagaaggtcaagactcctcttgtgcctgcggcgagaccgcgcacg |
| | | gtccacaggtgctggccctaccggtgtgaataagggcccgacgtcaggctcgtcgtta |
| | | aaccgagcccgtcacccacctgggcaaacgacgcccacgtacggtccacgtcgccctt |
| | | ca |
| 39 | Pegivirus A | tgtagcaatgcgcatattgctacttcggtacgcctaattggtaggcgcccggccgacc |
| | 1220 | ggccccgcaagggcctagtaggacgtgtgacaatgccatgcgggatcatgacactggg |
| | | gtgagcggaggcagcaccgaagtcgggtgaactcgactcccagtgcgaccacctggct |
| | | tggtcgttcatggagggcatgcccacgggaacgctgatcgtgcaaagggatgggtccc |
| | | tgcactggtgccatgcgcggcaccactccgtacagcctgatagggtggcggcgggccc |
| | | ccccagtgtgacgtccgtggagcgcaac |
| 40 | Pasivirus A 3 | attttctggccgtgtagctgcttttgaccagtggctctgggttacttagccaaatccc |
| | | ccttccttcacccttttaaatttgatggtctgtgttgtttgttttgtcttgtctaaat |
| | | aatatataagatccttcccgccgatacagacctcgacagtctggtgtaggagggttgg |
| | | tgttattaatttgccccagaagagtgaccgtgacacatagaaaccatgagtacatgtg |
| | | tatccgtggaggatcgcccgggactggattccatatcccattgccatcccaacaagcg |
| | | gagggtatacccactatgtgcgcgtttgcagtgggaatctgcaaatttagtcatactg |
| | | cctgatagggtgtgggcctgcactctggggtactcaggctgttcatataat |
| 41 | Sapelovirus | cccctccacccttaaggtggttgtatcccacataccccaccctcccttccaaagtgga |
| | | cggacaactggattttgactaacggcaagtctgaatggtatgatttggatacgtttaa |
| | | acggcagtagcgtggcgagctatggaaaaatcgcaattgtcgatagccatgttagtga |
| | | cgcgcttcggcgtgctcctttggtgattcggcgactggttacaggagagtaggcagtg |
| | | agctatgggcaaacctctacagtattacttagagggaatgtgcaattgagacttgacg |
| | | agcgtctctttgagatgtggcgcatgctcttggcattaccatagtgagcttccaggtt |
| | | gggaaacctggactgggcctatactacctgatagggtcgcggctggccgcctgtaact |
| | | agtatagtcagttgaaaccccccc |
| 42 | Rosavirus B | gtctctttagtgtctatgcttcagagagcggtgaactgacaccgttgcttcttgcaca |
| | | gcccttcgtgccggtctttccggttctcgacagcgttgggcatcatggctagttaggc |
| | | taagatagtggatgatctagtgaacagttttggattgtttggagttttgtagcgatgc |
| | | tagtagtgtgtgtggacctccccacgtggtaacacgtgccccacaggccaaaagccaa |
| | | ggtgttgaaagcacccctactagtcccagactcacccatctgggaactcctctcatga |
| | | aaaatcttagtaacttttgattcggctattcatcaacctctctagtcaagggctgaag |
| | | gatgcccggaaggtacccgcaggtaacgataagctcactgtggatctgatccggggct |
| | | ttggtgcgaccgtctgtccggcgtagccagagttaaaaaacgtctaggcccttccacc |
| | | ccaagggattggggtttccccaatcatttgaaagttcact |
| 43 | Bakunsa | ttttgaacgccacctcggagcgatatccggggaccccctcccctttttccttcctacc |
| | Virus | ttcttcccaaatttccctcttcccttgttattttggtttggatttcctggacatgact |
| | | cggacggatctatctcatttgctttgtgtctgctccaccagtggcatggtcgaaagat |
| | | catcaacactggacgtgtactgtaatggccaaacgtgcccacaggggaaaccatgccg |
| | | gtcgctgtagcggcgggtggacgtggtggacccctctccctgctcataaactttgggt |
| | | aggtgaagggttcaagcgacgcttgccgtgagggcgcatccggatgggggaaccaaca |
| | | aactaggctgtaatggccgacctcaggtggatgagctagggctgctgcaccaaaaggg |
| | | actcgattcgatatcccggcctggtagcctagtgcagtggactcgtagttgggaatct |
| | | acgactggcctagtacagggtgatagccccgtttcccacgcccacctgttgtagggac |
| | | acccccccc |
| 44 | Tremovirus A | tttgaaagaggcctccggagtgtccggaggctctctttcgacccaacccatactgggg |
| | | ggtgtgtgggaccgtacctggagtgcacggtatatatgcattcccgcatggcaagggc |
| | | gtgctaccttgccccttgacgcatggtatgcgtcatcatttgccttggttaagcccca |
| | | tagaaacgaggcgtcacgtgccgaaaatccctttgcgtttcacagaaccatcctaacc |
| | | atgggtgtagtatgggaatcgtgtatggggatgattaggatctctcgtagagggatag |
| | | gtgtgccattcaaatccagggagtactctggctctgacattgggacatttgatgtaac |
| | | cggacctggttcagtatccgggttgtcctgtattgttacggtgtatccgtcttggcac |
| | | actgaaagggtatttttgggtaatcctttcctactgcctgatagggtggcgtgcccgg |
| | | ccacgagagattaagggtagcaatttaaac |
| 45 | Swine | gcttttgaccagtggctctgggttacttagccaagtccctttctcttattttcactag |
| | Pasivirus 1 | tttatgttgtgtgttgtctgttttgttttgtttaaattgtatacaagatccttcccgc |
| | | cgacacagacctcgacagtctggtgtaggagggttggtgatattaatttgccccaaaa |
| | | gagtgaccgtgatacgtggaaaccatgagtacatgtgtatccgtggaggatcgcccgg |
| | | gactggattccatatcccattgccatcccaacaaacggagggtatacccaccacgtgc |
| | | gcgtttgcagtgggaatctgcaaatttagtcatactgcctgatagggtgtgggcctgc |
| | | actttggggtactcaggctgttcatataat |
| 46 | PLV-CHN | acatggggtatgttgtctgtcctgttttgttgaaacaatatataagatcctttccgcc |
| | | gatatagacctcgacagtctagtgtaggaggattggtgatagtaacttgccccagaag |
| | | agtgaccgtgacacatagaaaccatgagtacatgtgtatccgtggaggatcgcccggg |
| | | actggattccatatcccattgccatcccaacaaacggagggtatacccactatgtgcg |
| | | cgtttgcagtgggagcctgcaaatttagtcatactgcctgatagggtgtgggcctgca |
| | | ctctggggtactcaggctgtttatataat |
| 47 | Pasivirus A | tgaaaaagtggttgtgcagctggattttccggctgtgcaactgcttttgaccagtggc |
| | (longer) | tctgggttacttagccaaattcctttcccttatccctattggtttgtgttgtgtgttg |
| | | tttgttttgttttgtcttaactatatacaagatccttcccgccgatacagacctcgac |
| | | agtctggtgtaggagggttggtgttattaatttgccccaaaagagtgaccgtgacacg |
| | | tggaaaccatgagtacatgtgtatccgtggaggatcgcccgggactggattccatatc |
| | | ccattgccatcccaacaaacggagggtatacccaccacgtgcgcgtttgcagtgggaa |
| | | tctgcaaatttagtcatactgcctgatagggtgtgggcctgcactttggggtactcag |
| | | gctgtttatataat |
| 48 | Sicinivirus | gtgtcattaaggtgtgtttggaagttcgaattagctggtttgtggtgattagtagacc |
| | | ccctggaggtacccaattcggatctgaccagggacccgtgactataccgctccggtaa |
| | | ttcgggtttaaaacaatgaacgtcaccacacaattacttttctcattttattttcatc |
| | | attgtcttcctatttaccgattacactcgatttccttggatgttcctggagatttccc |
| | | tggttacctggaccctcattattgttgttgtttcacccagcgagctgtcccaattgct |
| | | tattatttgcgcttacaacttcgtcctaatatttttctggttgatcgggttgattgag |
| | | ctcccgggctatcctgccattcaac |
| 49 | Hepacivirus K | gggaacaatggtccgtccgcggaacgactctagccatgagtctagtacgagtgcgtgc |
| | | cacccattagcacaaaaaccactgactgagccacacccctcccggaatcctgagtaca |
| | | ggacattcgctcggacgacgcatgagcctccatgccgagaaaattgggtatacccacg |
| | | ggtaaggggtggccacccagcgggaatctgggggctggtcactgactatggtacagcc |
| | | tgatagggtgctgccgcagcgtcagtggtatgcggctgttcatggaac |
| 50 | Hepacivirus A | acctccgtgctaggcacggtgcgttgtcagcgttttgcgcttgcatgcgctacacgcg |
| | | tcgtccaacgcggagggaacttcacatcaccatgtgtcactccccctatggagggttc |
| | | caccccgcttacacggaaatgggttaaccatacccaaagtacgggtatgcgggtcctc |
| | | ctagggcccccccggcaggtcgagggagctggaattcgtgaattcgtgagtacacgaa |
| | | aatcgcggcttgaacgtctttgaccttcggagccgaaatttgggcgtgccccacgaag |
| | | gaaggcgggggcggtgttgggccgccgccccctttatcccacggtctgataggatgct |
| | | tgcgagggcacctgccggtctcgtagaccataggac |
| 51 | BVDVI | gtatacgagaatttgcctaggacctcgtttacaatatgggcaatctaaaattataatt |
| | | aggcctaagggacaaatcctcctcagcgaaggccgaaaagaggctagccatgccctta |
| | | gtaggactagcaaaataaggggggtagcaacagtggtgagttcgttggatggctgaag |
| | | ccctgagtacagggtagtcgtcagtggttcgacgcttcggaggacaagcctcgagata |
| | | ccacgtggacgagggcatgcccacagcacatcttaacctggacgggggtcgttcaggt |
| | | gaaaacggtttaaccaaccgctacgaatacagcctgatagggtgctgcagaggcccac |
| | | tgtattgctactgaaaatctctgctgtacatggcac |
| 52 | Border | gtatacgggagtagctcatgcccgtatacaaaattggatattccaaaactcgattggg |
| | Disease Virus | ttagggagccctcctagcgacggccgaaccgtgttaaccatacacgtagtaggactag |
| | | cagacgggaggactagccatcgtggtgagatccctgagcagtctaaatcctgagtaca |
| | | ggatagtcgtcagtagttcaacgcaggcacggttctgccttgagatgctacgtggacg |
| | | agggcatgcccaagacttgctttaatctcggcgggggtcgccgaggtgaaaacaccta |
| | | acggtgttggggttacagcctgatagggtgctgcagaggcccacgaataggctagtat |
| | | aaaaatctctgctgtacatggcac |
| 53 | BVDV2 | gtatacgagattagctaaagtactcgtatatggattggacgtcaacaaatttttaatt |
| | | ggcaacgtagggaaccttcccctcagcgaaggccgaaaagaggctagccatgcccttt |
| | | agtaggactagcaaaagtagggggactagcggtagcagtgagttcgttggatggccga |
| | | acccctgagtacaggggagtcgtcaatggttcgacactccattagtcgaggagtctcg |
| | | agatgccatgtggacgagggcatgcccacggcacatcttaacccatggggggttgcat |
| | | gggtgaaagcgctaatcgtggcgttatggacacagcctgatagggtgtagcagagacc |
| | | tgctattccgctagtaaaaaactctgctgtacatggcac |
| 54 | CSFV-PK15C | gtatacgaggttagttcattctcgtatgcattattggacaaatcaaaatttcaatttg |
| | | gttcagggcctccctccagcgacggccgaactgggctagccatgcccatagtaggact |
| | | agcaaacggagggactagccgtagtggcgagctccctgggtgttctaagtcctgagta |
| | | caggacagtcgtcagtagttcgacgtgagcagaagcccacctcgagatgctatgtgga |
| | | cgagggcatgcccaagacgcaccttaaccctagcgggggtcgctagggtgaaatcaca |
| | | ccacgtgatgggagtccgacctgatagggtgctgcagaggctcactattaggctagta |
| | | taaaaatctctgctgtacatggcac |
| 55 | SF573 | aaaaccgaccccagagatcagaaagtcgttgacgcgatcttttattagaggacgttgc |
| | Dicistrovirus | gctggcgcgagctttaattagcagacgccaaaaataaacaacaaaatgctgatcgcga |
| | | gacttaattgtcagacgattggccaaatccgatgtgatctttgctgctcccagattgc |
| | | cgaaataggagtagtag |
| 56 | Hubei | ccccaaaaccccccccttaaactcaacactgtagtggattcattttccgttgcaaaac |
| | Picorna-like | aaaacattactacccgcatttatgtaggctctgtgttttctatgcgaccgttacatta |
| | Virus | atctctactctgacccactagtttataaaaccgaagacctgaatgaaacgattttcct |
| | | tcttttcaacctctaacgaacctctgacggcttgagaaacctgaagttagtaattatg |
| | | tttaaaagaaaggaaagtcaaacgcgatgactcttacatccctattccataccgttgc |
| | | tccacaatgtgagcgatgcgaggtcgggactgcagtattaggggaacgagctacatgg |
| | | agagttaattatctctcccctcctacgggagtctcatgtgagctgtagaaagcggttg |
| | | gcacctctcgttacctcgcctgtacatgatcc |
| 57 | CRPV | aaaagcaaaaatgtgatcttgcttgtaaatacaattttgagaggttaataaattacaa |
| | | gtagtgctatttttgtatttaggttagctatttagctttacgttccaggatgcctagt |
| | | ggcagccccacaatatccaggaagccctctctgcggtttttcagattaggtagtcgaa |
| | | aaacctaagaaatttacct |
| 58 | Salivirus A | tttcctcctttcgaccgccttacggcaggcgggtccgcggacggcttcggcctacccg |
| | BN5 | cgacaagaatgccgtcatctgtccttatcacccatattctttcccttcccccgcaacc |
| | | atcacgcttactcgcgcacgtgttgagtggcacgtgcgttgtccaaacagttacactc |
| | | acacccttgggggggtttgtcccgccctcgggttcctcgcggaaccctccctcttctc |
| | | tctccctttctatccgccttcactttccataactacagtgctttggtaggtaagcatc |
| | | ctgaccccccgcggaagctgccaacgtggcaactgtggggatccaggcaggttatcaa |
| | | aggcacccggtctttccgccttcaggagtatccctgccggtgaattccgacagggctc |
| | | tgcttggtgccaacctcccccaaatgcgcgctgcgggagtgctcttccccaactcatc |
| | | ttagtaacctctcatgtgtgtgcttggtcagcatatctgaggcgacgttccgctgtcc |
| | | cagaccagtccagcaatggacgggccagtgtgcgtagtcgctttccggtttcccggcg |
| | | catgtttggcgaaacgctgaggtaaggttggtgtgcccaatgcccgtaatttggtgac |
| | | acctcaagaccacccaggaatgccagggaggtaccccacttcggtgggatctgaccct |
| | | gggctaattgtctacggtggttcttcttgcttccacttctcttttttctggcatg |
| 59 | Salivirus A | tatggcaggcgggcttgtggacggcttcggcccacccacagcaagaatgccatcatct |
| | BN2 | gtcctcacccccatgtttcccctttctttccctgcaaccgttacgcttactcgcaggt |
| | | gcatttgagtggtgcacgtgttgaataaacagctacactcacatggggggggttttcc |
| | | cgccctgcggcctctcgcgaggcccacccctccccttcctcccataactacagtgctt |
| | | tggtaggtaagcatcctgatcccccgcggaagctgctcacgtggcaactgtggggacc |
| | | cagacaggttatcaaaggcacccggtctttccgccttcaggagtatccctgctagtga |
| | | attctagtagggctctgcttggtgccaacctcccccaaatgcgcgctgcgggagtgct |
| | | cttccccaactcaccctagtatcctctcatgtgtgtgcttggtcagcatatctgagac |
| | | gatgttccgctgtcccagaccagtccagtaatggacgggccagtgtgcgtagtcgtct |
| | | tccggcttttccggcgcatgtttggtgaaccggtggggtaaggttggtgtgcccaacg |
| | | cccgtactttggtgatacctcaagaccacccaggaatgccagggaggtaccccgcttc |
| | | acagcgggatctgaccctgggctaattgtctacggtggttcttcttgcttccacttct |
| | | ttctactgttc |
| 60 | Salivirus A | tttcgaccgccttatggcaggcgggcttgtggacggcttcggcccacccacagcaaga |
| | 02394 | atgccatcatctgtcctcacccccatttctcccctccttcccctgcaaccattacgct |
| | | tactcgcatgtgcattgagtggtgcacgtgttgaacaaacagctacactcacgtgggg |
| | | gcgggttttcccgcccttcggcctctcgcgaggcccacccttccccttcctcccataa |
| | | ctacagtgctttggtaggtaagcatcctgatcccccgcggaagctgctcgcgtggcaa |
| | | ctgtggggacccagacaggttatcaaaggcacccggtctttccgcctccaggagtatc |
| | | cctgctagtgaattctagtggggctctgcttggtgccaacctcccccaaatgcgcgct |
| | | ggggagtgctcttccccaactcaccctagtatcctctcatgtgtgtgcttggtcagca |
| | | tatctgagacgatgttccgctgtcccagaccagtccagcaatggacgggccagtgtgc |
| | | gtagtcgtcttccggcttgtccggcgcatgtttggtgaaccggtggggtaaggttggt |
| | | gtgcccaacgcccgtactttggtgacaactcaagaccacccaggaatgccagggaggt |
| | | accccgcctcacggcgggatctgaccctgggctaattgtctacggtggttcttcttgc |
| | | ttccatttctttcttctgttc |
| 61 | Salivirus A | tatggcaggcgggcttgtggacggtttcggcccacccacagcaagaatgccatcatct |
| | GUT | gtcctcacccccaattttccctttcttcccctgcaatcatcacgcttactcgcatgtg |
| | | cattgagtggtgcatgtgttgaacaaacagctacactcacatggggggggttttcccg |
| | | ccctacggcctctcgcgaggcccacccttcccctccccttataactacagtgtttggc |
| | | aggtaagcatcctgatcccccgcggaagctgctcacgtggcaactgtggggacccaga |
| | | caggttatcaaaggcacccggtctttccgccttcaggagcatccccactagtgaattc |
| | | tagtggggctctgcttggtgccaacctcccccaaatgcgcgctgcgggagtgctcttc |
| | | cccaacccatcctagtatcctctcatgtgtgtgcttggtcagcatatctgagacgacg |
| | | ttccgctgtcccagaccagtccagtaatggacgggccagtgtgcgtagtcgtcttccg |
| | | gcttgtccggcgcatgtttggtgaaccggtggggtaaggttggtgtgcccaacgcccg |
| | | tactttggtgacacctcaagaccacccaggaatgccagggaggtaccccgcctcacgg |
| | | cgggatctgaccctgggctaattgtctacggtggttcttcttgcttccacttctttct |
| | | t |
| 62 | Salivirus A | ttctcctgcaaccattacgcttaatcgcatgtgcattgagtggtgcatgtgttgaaca |
| | CH | aacagctacaatcacatggggggggttttcccgccccacggcttctcgcgaggcccat |
| | | ccctcccttttctcccataactacagtgctttggtaggtaagcatcccgatctcccgc |
| | | ggaagctgctcacgtggcaactgtggggacccagacaggttatcaaaggcacccggtc |
| | | tttccgccttcaggagtatccctgctagcgaattctagtagggctctgcttggtgcca |
| | | acctctcccaaatgcgcgctgcgggagtgctcttccccaaatcaccccagtatcctct |
| | | catgtgtgtgcctggtcagcatatctgagacgatgttccgctgtcccagaccagtcca |
| | | gtaatggacgggccagtgtgcgtagtcgtcctccggcttgtccggcgcatgtttggtg |
| | | aaccggtggggtaaggttggtgtgcccaacgcccgtaatcaggggatacctcaaggca |
| | | cccaggaatgccagggaggtatcccgcctcacagcgggatctgaccctggggtaaatg |
| | | tctgcggggggtcctcttggcccaattctcagtaattttcagg |
| 63 | Salivirus A | tctgtcctcaccccatcttcccttctttcctgcaccgttacgcttactcgcatgtgca |
| | SZ1 | ttgagtggtgcacgtgcttgaacaaacagctacactcacatggggggggttttcccgc |
| | | cctgcggcctctcgcgaggcccacccctccccttcctcccataactacagtgctttgg |
| | | taggtaagcatcctgatcccccgcggaagctgctcacgtggcaactgtggggacccag |
| | | acaggttatcaaaggcacccggtctttccgccttcaggagtatccctgctagtgaatt |
| | | ctagtagggctctgcttggtgccaacctcccccaaatgcgcgctgcgggagtgctctt |
| | | ccccaactcaccctagtatcctctcatgtgtgtgcttggtcagcatatctgagacgat |
| | | gttccgctgtcccagaccagtccagtaatggacgggccagtgtgcgtagtcgtcttcc |
| | | ggcttgtccggcgcatgtttggtgaaccggtggggtaaggttggtgtgcccaacgccc |
| | | gtactttggtgatacctcaagaccacccaggaatgccagggaggtaccccgcttcaca |
| | | ggggatctgaccctgggctaattgtctacggtggttcttcttgcttccacttctttct |
| | | actgttcatg |
| 64 | Salivirus FHB | acatggggggtctgcggacggcttcggcccacccgcgacaagaatgccgtcatctgtc |
| | | ctcattacccgtattccttcccttcccccgcaaccaccacgcttactcgcgcacgtgt |
| | | tgagtggcacgtgcgttgtccaaacagctacacccacacccttcggggcgggtttgtc |
| | | ccgccctcgggttcctcgcggaacccccccctccctctctctctttctatccgccctc |
| | | acttcccataactacagtgctttggtaggtgagcaccctgaccccccgcggaagctgc |
| | | taacgtggcaactgtggggatccaggcaggttatcaaaggcacccggtctttccgcct |
| | | tcaggagtatctctgccggtgaattccggtagggctctgcttggtgccaacctccccc |
| | | aaatgcgcgctgcgggagtgctcttccccaactcatcttagtaacctctcatgtgtgt |
| | | gcttggtcagcatatctgaggcgacgttccgctgtcccagaccagtccagcaatggac |
| | | gggccagtgtgcgtagtcgctttccggttttccggcgcatgtttggcgaaacgctgag |
| | | gtaaggttggtgtgcccaacgcccgtaatttggtgatacctcaagaccacccaggaat |
| | | gccagggaggtaccccacttcggtgggatctgaccctgggctaattgtctacggtggt |
| | | tcttcttgcttccacttctcttttttctggcatg |
| 65 | CVB3 | ttaaaacagcctgtgggttgatcccacccacaggcccattgggcgctagcactctggt |
| | | atcacggtacctttgtgcgcctgttttataccccctcccccaactgtaacttagaagt |
| | | aacacacaccgatcaacagtcagcgtggcacaccagccacgttttgatcaagcacttc |
| | | tgttaccccggactgagtatcaatagactgctcacgcggttgaaggagaaagcgttcg |
| | | ttatccggccaactacttcgaaaaacctagtaacaccgtggaagttgcagagtgtttc |
| | | gctcagcactaccccagtgtagatcaggtcgatgagtcaccgcattccccacgggcga |
| | | ccgtggcggtggctgcgttggcggcctgcccatggggaaacccatgggacgctctaat |
| | | acagacatggtgcgaagagtctattgagctagttggtagtcctccggcccctgaatgc |
| | | ggctaatcctaactgcggagcacacaccctcaagccagagggcagtgtgtcgtaacgg |
| | | gcaactctgcagcggaaccgactactttgggtgtccgtgtttcattttattcctatac |
| | | tggctgcttatggtgacaattgagagatcgttaccatatagctattggattggccatc |
| | | cggtgactaatagagctattatatatccctttgttgggtttataccacttagcttgaa |
| | | agaggttaaaacattacaattcattgttaagttgaatacagcaaa |
| 66 | CVB1 | ttaaaacagcctgtgggttgttcccacccacaggcccattgggcgctagcactctggt |
| | | atcacggtacctttgtgcgcctgttttacatcccctccccaaattgtaatttagaagt |
| | | ttcacacaccgatcattagcaagcgtggcacaccagccatgttttgatcaagcacttc |
| | | tgttaccccggactgagtatcaatagaccgctaacgcggttgaaggagaaaacgttcg |
| | | ttacccggccaactacttcgaaaaacctagtaacaccatggaagttgcggagtgtttc |
| | | gctcagcactaccccagtgtagatcaggtcgatgagtcaccgcgttccccacgggcga |
| | | ccgtggcggtggctgcgttggcggcctgcctacggggaaacccgtaggacgctctaat |
| | | acagacatggtgcgaagagtctattgagctagttggtaatcctccggcccctgaatgc |
| | | ggctaatcctaactgcggagcacataccctcaaaccagggggcagtgtgtcgtaacgg |
| | | gcaactctgcagcggaaccgactactttgggtgtccgtgtttcattttattcctatac |
| | | tggctgcttatggtgacaattgacaggttgttaccatatagttattggattggccatc |
| | | cggtgactaacagagcaattatatatctctttgttgggtttataccacttagcttgaa |
| | | agaggttaaaacactacatctcatcattaaactaaatacaacaaa |
| 67 | Echovirus 7 | ttaaaacagcctgtgggttgttcccacccacagggcccattgggcgtcagcaccctgg |
| | | tatcacggtacctttgtgcgcctgttttatatcccttcccccaattgtaacttagaag |
| | | aaacacacaccgatcaacagcaagcgtggcacaccagccatgttttggtcaagcactt |
| | | ctgttaccccggactgagtatcaatagactgctcacgcggttgaaggagaaagcgtcc |
| | | gttatccggccagctacttcgagaaacctagtaacaccatggaagttgcggagtgttt |
| | | cgctcagcactaccccagtgtagatcaggtcgatgagtcaccgctttccccacgggcg |
| | | accgtggcggtggctgcgttggcggcctgcctatgggggaacccataggacgctctaa |
| | | tacagacatggtgcgaagagtctattgagctagctggtattcctccggcccctgaatg |
| | | cggctaatcctaactgtggagcacatgcccctaatccaaggggtagtgtgtcgtaatg |
| | | agcaattccgcagcggaaccgactactttgggtgtccgtgtttcctcttattcttgta |
| | | ctggctgcttatggtgacaattgagagattgttaccatatagctattggattggccat |
| | | ccggtgactaatagagctattgtgtatctctttgttggatttgtaccacttaatttga |
| | | aagaaatcaggacactacgctacattttactattgaacaccgcaaa |
| 68 | CVB5 | ttaaaacagcctgtgggttgtacccacccacagggcccactgggcgctagcactctgg |
| | | tatcacggtacctttgtgcgcctgttttatgcccccttcccccaattgaaacttagaa |
| | | gttacacacaccgatcaacagcgggcgtggcataccagccgcgtcttgatcaagcact |
| | | cctgtttccccggaccgagtatcaatagactgctcacgcggttgaaggagaaaacgtt |
| | | cgttacccggctaactacttcgagaaacctagtagcatcatgaaagttgcgaagcgtt |
| | | tcgctcagcacatccccagtgtagatcaggtcgatgagtcaccgcattccccacgggc |
| | | gaccgtggcggtggctgcgttggcggcctgcctacggggcaacccgtaggacgcttca |
| | | atacagacatggtgcgaagagtcgattgagctagttagtagtcctccggcccctgaat |
| | | ccggctaatcctaactgcggagcacataccctcaacccagggggcattgtgtcgtaac |
| | | gggtaactctgcagcggaaccgactactttgggtgtccgtgtttccttttattcttat |
| | | aatggctgcttatggtgacaattgaaagattgttaccatatagctattggattggcca |
| | | tccggtgtctaacagagctattatatacctctttgttggatttgtaccacttgatcta |
| | | aaggaagtcaagacactacaattcatcatacaattgaacacagcaaa |
| 69 | EVA71 | ttaaaacagcctgtgggttgcacccactcacagggcccactgggcgcaagcactctgg |
| | | cacttcggtacctttgtgcgcctgttttatatcccctcccccaatgaaatttagaagc |
| | | agcaaaccccgatcaatagcaggcataacgctccagttatgtcttgatcaagcacttc |
| | | tgtttccccggactgagtatcaatagactgctcacgcggttgaaggagaaaacgttcg |
| | | ttatccggctaactacttcggaaagcctagtaacaccatggaagttgcggagagtttc |
| | | gttcagcacttccccagtgtagatcaggtcgatgagtcaccgcattccccacgggcga |
| | | ccgtggcggtggctgcgttggcggcctgcccatggggtaacccatgggacgctctaat |
| | | acggacatggtgtgaagagtctactgagctagttagtagtcctccggcccctgaatgc |
| | | ggctaatcccaactgcggagcacacgcccacaagccagtgggtagtgtgtcgtaacgg |
| | | gcaactctgcagcggaaccgactactttgggtgtccgtgtttccttttattcttatgt |
| | | tggctgcttatggtgacaattaaagagttgttaccatatagctattggattggccatc |
| | | cggtgtgcaacagagcgatcgtttacctatttattggttttgtaccattgacactgaa |
| | | gtctgtgatcacccttaattttatcttaaccctcaacacagccaaac |
| 70 | CVA3 | ttaaaacagcctgtgggttgtacccacccacagggcccactgggcgctagcacactgg |
| | | tattacggtacctttgtgcgcctgttttataccccccccaacctcgaaacttagaagt |
| | | aaagcaaacccgatcaatagcaggtgcggcgcaccagtcgcatcttgatcaagcactt |
| | | ctgtaaccccggaccgagtatcaatagactgctcacgcggttgaaggagaaaacgttc |
| | | gttacccggctaactacttcgagaaacccagtagcatcatgaaagttgcagagtgttt |
| | | cgctcagcactacccccgtgtagatcaggccgatgagtcaccgcacttccccacgggc |
| | | gaccgtggcggtggctgcgttggcggcctgcctatggggcaacccataggacgctcta |
| | | atacggacatggtgcgaagagtctattgagctagttagtagtcctccggcccctgaat |
| | | gcggctaatcctaactgcggagcacatacccttaatccaaagggcagtgtgtcgtaac |
| | | gggtaactctgcagcggaaccgactactttgggtgtccgtgtttccttttaattttta |
| | | ctggctgcttatggtgacaattgaggaattgttgccatatagctattggattggccat |
| | | ccggtgactaacagagctattgtgttccaatttgttggatttaccccgctcacactca |
| | | cagtcgtaagaacccttcattacgtgttatttctcaactcaagaaa |
| 71 | CVA12 | ttaaaacagcctgtgggttgtacccacccacagggcccactgggcgctagcactctgg |
| | | tactacggtacctttgtgtgcctgttttaagcccctaccccccactcgtaacttagaa |
| | | ggcttctcacactcgatcaatagtaggtgtggcacgccagtcacaccgtgatcaagca |
| | | cttctgttaccccggtctgagtaccaataagctgctaacgcggctgaaggggaaaacg |
| | | atcgttatccggctaactacttcgagaaacccagtaccaccatgaacgttgcagggtg |
| | | tttcgctcggcacaaccccagtgtagatcaggtcgatgagtcaccgtattccccacgg |
| | | gcgaccgtggcggtggctgcgttggcggcctgcccatggggtgacccatgggacgctc |
| | | taatactgacatggtgcgaagagtctattgagctagttagtagtcctccggcccctga |
| | | atgcggctaatcctaactgcggagcacatacccttaatccaaagggcagtgtgtcgta |
| | | acgggcaactctgcagcggaaccgactactttgggtgtccgtgtttccttttattctt |
| | | acattggctgcttatggtgacaattgaaaagttgttaccatatagctattggattggc |
| | | catccggtgacaaatagagctattgtatatctttttgttggttacgtaccccttaatt |
| | | acaaagtggtttcaactttgaaatacatcctaacactaaattgtagaaa |
| 72 | EV24 | ttaaaacagcctgtgggttgcacccacccacagggcccacagggcgctagcactctgg |
| | | tatcacggtacctttgtgcgcctgttttattaccccttccccaattgaaaattagaag |
| | | caatgcacaccgatcaacagcaggcgtggcgcaccagtcacgtctcgatcaagcactt |
| | | ctgtttccccggaccgagtatcaatagactgctcacgggttgaaggagaaagtgttcg |
| | | ttatccggctaaccacttcgagaaacccagtaacaccatgaaagttgcagggtgtttc |
| | | gctcagcacttccccagtgtagatcaggtcgatgagtcaccgcgttccccacgggcga |
| | | ccgtggcggtggctgcgttggcggcctgcctatgggttaacccataggacgctctaat |
| | | acagacatggtgcgaagagtttattgagctggttagtatccctccggcccctgaatgc |
| | | ggctaatcctaactgcggagcacgtgcctccaatccagggggttgcatgtcgtaacgg |
| | | gtaactctgcagcggaaccgactactttgggtgtccgtgtttccttttattcttatac |
| | | tggctgcttatggtgacaatcgaggaattgttaccatatagctattggattggccatc |
| | | cggtgtctaacagagcgattatatacctctttgttggatttatgcagctcaataccac |
| | | caactttaacacattgaaatatatcttaaagttaaacacagcaaa |

In some embodiments, an IRES of the invention is an IRES having a sequence as listed in Table 1 (SEQ ID NO: 1-72). In some embodiments, an IRES is a Salivirus IRES. In some embodiments, an IRES is a Salivirus SZ1 IRES.

TABLE 2
Anabaena permutation site
5′ intron fragment sequences.

| SEQ | Permu- | |
|---|---|---|
| ID | tation | |
| NO: | site | Sequence |
| 73 | L2-1 | GAAGAAATTCTTTAAGTGGATGCTCTCAAACTCAGG |
| | | GAAACCTAAATCTAGTTATAGACAAGGCAATCCTGA |
| | | GCCAAGCCGAAGTAGTAATTAGTAAGTTAACAATAG |
| | | ATGACTTACAACTAATCGGAAGGTGCAGAGACTCGA |
| | | CGGGAGCTACCCTAACGTCAAGACGAGGGTAAAGA |
| | | GAGAGTCCAATTCTCAAAGCCAATAGGCAGTAGCGA |
| | | AAGCTGCAAGAGAATGAAAATCCGT |
| 74 | L2-2 | AAGAAATTCTTTAAGTGGATGCTCTCAAACTCAGGG |
| | | AAACCTAAATCTAGTTATAGACAAGGCAATCCTGAG |
| | | CCAAGCCGAAGTAGTAATTAGTAAGTTAACAATAGA |
| | | TGACTTACAACTAATCGGAAGGTGCAGAGACTCGAC |
| | | GGGAGCTACCCTAACGTCAAGACGAGGGTAAAGAG |
| | | AGAGTCCAATTCTCAAAGCCAATAGGCAGTAGCGAA |
| | | AGCTGCAAGAGAATGAAAATCCGT |
| 75 | L2-3 | AGAAATTCTTTAAGTGGATGCTCTCAAACTCAGGGA |
| | | AACCTAAATCTAGTTATAGACAAGGCAATCCTGAGC |
| | | CAAGCCGAAGTAGTAATTAGTAAGTTAACAATAGAT |
| | | GACTTACAACTAATCGGAAGGTGCAGAGACTCGACG |
| | | GGAGCTACCCTAACGTCAAGACGAGGGTAAAGAGA |
| | | GAGTCCAATTCTCAAAGCCAATAGGCAGTAGCGAAA |
| | | GCTGCAAGAGAATGAAAATCCGT |
| 76 | L5-1 | GTTATAGACAAGGCAATCCTGAGCCAAGCCGAAGTA |
| | | GTAATTAGTAAGTTAACAATAGATGACTTACAACTA |
| | | ATCGGAAGGTGCAGAGACTCGACGGGAGCTACCCTA |
| | | ACGTCAAGACGAGGGTAAAGAGAGAGTCCAATTCTC |
| | | AAAGCCAATAGGCAGTAGCGAAAGCTGCAAGAGAA |
| | | TGAAAATCCGT |
| 77 | L5-2 | TTATAGACAAGGCAATCCTGAGCCAAGCCGAAGTAG |
| | | TAATTAGTAAGTTAACAATAGATGACTTACAACTAA |
| | | TCGGAAGGTGCAGAGACTCGACGGGAGCTACCCTAA |
| | | CGTCAAGACGAGGGTAAAGAGAGAGTCCAATTCTCA |
| | | AAGCCAATAGGCAGTAGCGAAAGCTGCAAGAGAAT |
| | | GAAAATCCGT |
| 78 | L5-3 | TATAGACAAGGCAATCCTGAGCCAAGCCGAAGTAGT |
| | | AATTAGTAAGTTAACAATAGATGACTTACAACTAAT |
| | | CGGAAGGTGCAGAGACTCGACGGGAGCTACCCTAAC |
| | | GTCAAGACGAGGGTAAAGAGAGAGTCCAATTCTCAA |
| | | AGCCAATAGGCAGTAGCGAAAGCTGCAAGAGAATG |
| | | AAAATCCGT |
| 79 | L5-4 | ATAGACAAGGCAATCCTGAGCCAAGCCGAAGTAGTA |
| | | ATTAGTAAGTTAACAATAGATGACTTACAACTAATC |
| | | GGAAGGTGCAGAGACTCGACGGGAGCTACCCTAACG |
| | | TCAAGACGAGGGTAAAGAGAGAGTCCAATTCTCAAA |
| | | GCCAATAGGCAGTAGCGAAAGCTGCAAGAGAATGA |
| | | AAATCCGT |
| 80 | L5-5 | TAGACAAGGCAATCCTGAGCCAAGCCGAAGTAGTAA |
| | | TTAGTAAGTTAACAATAGATGACTTACAACTAATCG |
| | | GAAGGTGCAGAGACTCGACGGGAGCTACCCTAACGT |
| | | CAAGACGAGGGTAAAGAGAGAGTCCAATTCTCAAA |
| | | GCCAATAGGCAGTAGCGAAAGCTGCAAGAGAATGA |
| | | AAATCCGT |
| 81 | L6-1 | ACAATAGATGACTTACAACTAATCGGAAGGTGCAGA |
| | | GACTCGACGGGAGCTACCCTAACGTCAAGACGAGGG |
| | | TAAAGAGAGAGTCCAATTCTCAAAGCCAATAGGCAG |
| | | TAGCGAAAGCTGCAAGAGAATGAAAATCCGT |
| 82 | L6-2 | CAATAGATGACTTACAACTAATCGGAAGGTGCAGAG |
| | | ACTCGACGGGAGCTACCCTAACGTCAAGACGAGGGT |
| | | AAAGAGAGAGTCCAATTCTCAAAGCCAATAGGCAGT |
| | | AGCGAAAGCTGCAAGAGAATGAAAATCCGT |
| 83 | L6-3 | AATAGATGACTTACAACTAATCGGAAGGTGCAGAGA |
| | | CTCGACGGGAGCTACCCTAACGTCAAGACGAGGGTA |
| | | AAGAGAGAGTCCAATTCTCAAAGCCAATAGGCAGTA |
| | | GCGAAAGCTGCAAGAGAATGAAAATCCGT |
| 84 | L6-4 | ATAGATGACTTACAACTAATCGGAAGGTGCAGAGAC |
| | | TCGACGGGAGCTACCCTAACGTCAAGACGAGGGTAA |
| | | AGAGAGAGTCCAATTCTCAAAGCCAATAGGCAGTAG |
| | | CGAAAGCTGCAAGAGAATGAAAATCCGT |
| 85 | L6-5 | TAGATGACTTACAACTAATCGGAAGGTGCAGAGACT |
| | | CGACGGGAGCTACCCTAACGTCAAGACGAGGGTAAA |
| | | GAGAGAGTCCAATTCTCAAAGCCAATAGGCAGTAGC |
| | | GAAAGCTGCAAGAGAATGAAAATCCGT |
| 86 | L6-6 | AGATGACTTACAACTAATCGGAAGGTGCAGAGACTC |
| | | GACGGGAGCTACCCTAACGTCAAGACGAGGGTAAA |
| | | GAGAGAGTCCAATTCTCAAAGCCAATAGGCAGTAGC |
| | | GAAAGCTGCAAGAGAATGAAAATCCGT |
| 87 | L6-7 | GATGACTTACAACTAATCGGAAGGTGCAGAGACTCG |
| | | ACGGGAGCTACCCTAACGTCAAGACGAGGGTAAAG |
| | | AGAGAGTCCAATTCTCAAAGCCAATAGGCAGTAGCG |
| | | AAAGCTGCAAGAGAATGAAAATCCGT |
| 88 | L6-8 | ATGACTTACAACTAATCGGAAGGTGCAGAGACTCGA |
| | | CGGGAGCTACCCTAACGTCAAGACGAGGGTAAAGA |
| | | GAGAGTCCAATTCTCAAAGCCAATAGGCAGTAGCGA |
| | | AAGCTGCAAGAGAATGAAAATCCGT |
| 89 | L6-9 | TGACTTACAACTAATCGGAAGGTGCAGAGACTCGAC |
| | | GGGAGCTACCCTAACGTCAAGACGAGGGTAAAGAG |
| | | AGAGTCCAATTCTCAAAGCCAATAGGCAGTAGCGAA |
| | | AGCTGCAAGAGAATGAAAATCCGT |
| 90 | L8-1 | CAAGACGAGGGTAAAGAGAGAGTCCAATTCTCAAA |
| | | GCCAATAGGCAGTAGCGAAAGCTGCAAGAGAATGA |
| | | AAATCCGT |
| 91 | L8-2 | AAGACGAGGGTAAAGAGAGAGTCCAATTCTCAAAG |
| | | CCAATAGGCAGTAGCGAAAGCTGCAAGAGAATGAA |
| | | AATCCGT |
| 92 | L8-3 | AGACGAGGGTAAAGAGAGAGTCCAATTCTCAAAGCC |
| | | AATAGGCAGTAGCGAAAGCTGCAAGAGAATGAAAA |
| | | TCCGT |
| 93 | L8-4 | GACGAGGGTAAAGAGAGAGTCCAATTCTCAAAGCCA |
| | | ATAGGCAGTAGCGAAAGCTGCAAGAGAATGAAAAT |
| | | CCGT |
| 94 | L8-5 | ACGAGGGTAAAGAGAGAGTCCAATTCTCAAAGCCAA |
| | | TAGGCAGTAGCGAAAGCTGCAAGAGAATGAAAATC |
| | | CGT |
| 95 | L9a-1 | AATAGGCAGTAGCGAAAGCTGCAAGAGAATGAAAA |
| | | TCCGT |
| 96 | L9a-2 | ATAGGCAGTAGCGAAAGCTGCAAGAGAATGAAAAT |
| | | CCGT |
| 97 | L9a-3 | TAGGCAGTAGCGAAAGCTGCAAGAGAATGAAAATC |
| | | CGT |
| 98 | L9a-4 | AGGCAGTAGCGAAAGCTGCAAGAGAATGAAAATCC |
| | | GT |
| 99 | L9a-5 | GGCAGTAGCGAAAGCTGCAAGAGAATGAAAATCCG |
| | | T |
| 100 | L9-1 | GAAAGCTGCAAGAGAATGAAAATCCGT |
| 101 | L9-2 | AAAGCTGCAAGAGAATGAAAATCCGT |
| 102 | L9-3 | AAGCTGCAAGAGAATGAAAATCCGT |
| 103 | L9-4 | AGCTGCAAGAGAATGAAAATCCGT |
| 104 | L9-5 | GCTGCAAGAGAATGAAAATCCGT |
| 105 | L9-6 | CTGCAAGAGAATGAAAATCCGT |
| 106 | L9-7 | AAGAGAATGAAAATCCGT |
| 107 | L9-8 | AGAGAATGAAAATCCGT |
| 108 | L9-9 | GAGAATGAAAATCCGT |
| 109 | L9a-6 | GCAGTAGCGAAAGCTGCAAGAGAATGAAAATCCGT |
| 110 | L9a-7 | AGTAGCGAAAGCTGCAAGAGAATGAAAATCCGT |
| 111 | L9a-8 | GTAGCGAAAGCTGCAAGAGAATGAAAATCCGT |

In some embodiments, a 5′ intron fragment is a fragment having a sequence listed in Table 2. Typically, a construct containing a 5′ intron fragment listed in Table 2 will contain a corresponding 3′ intron fragment as listed in Table 3 (e.g., both representing fragments with the L9a-8 permutation site).

TABLE 3
Anabaena permutation site
3′ intron fragment sequences.

| SEQ | Permu- | |
|---|---|---|
| ID | tation | |
| NO: | site | Sequence |
| 112 | L2-1 | ACGGACTTAAATAATTGAGCCTTAAA |
| 113 | L2-2 | ACGGACTTAAATAATTGAGCCTTAAAG |
| 114 | L2-3 | ACGGACTTAAATAATTGAGCCTTAAAGA |
| 115 | L5-1 | ACGGACTTAAATAATTGAGCCTTAAAGAAGAAATTC |
| | | TTTAAGTGGATGCTCTCAAACTCAGGGAAACCTAAA |
| | | TCTA |
| 116 | L5-2 | ACGGACTTAAATAATTGAGCCTTAAAGAAGAAATTC |
| | | TTTAAGTGGATGCTCTCAAACTCAGGGAAACCTAAA |
| | | TCTAG |
| 117 | L5-3 | ACGGACTTAAATAATTGAGCCTTAAAGAAGAAATTC |
| | | TTTAAGTGGATGCTCTCAAACTCAGGGAAACCTAAA |
| | | TCTAGT |
| 118 | L5-4 | ACGGACTTAAATAATTGAGCCTTAAAGAAGAAATTC |
| | | TTTAAGTGGATGCTCTCAAACTCAGGGAAACCTAAA |
| | | TCTAGTT |
| 119 | L5-5 | ACGGACTTAAATAATTGAGCCTTAAAGAAGAAATTC |
| | | TTTAAGTGGATGCTCTCAAACTCAGGGAAACCTAAA |
| | | TCTAGTTA |
| 120 | L6-1 | ACGGACTTAAATAATTGAGCCTTAAAGAAGAAATTC |
| | | TTTAAGTGGATGCTCTCAAACTCAGGGAAACCTAAA |
| | | TCTAGTTATAGACAAGGCAATCCTGAGCCAAGCCGA |
| | | AGTAGTAATTAGTAAGTTA |
| 121 | L6-2 | ACGGACTTAAATAATTGAGCCTTAAAGAAGAAATTC |
| | | TTTAAGTGGATGCTCTCAAACTCAGGGAAACCTAAA |
| | | TCTAGTTATAGACAAGGCAATCCTGAGCCAAGCCGA |
| | | AGTAGTAATTAGTAAGTTAA |
| 122 | L6-3 | ACGGACTTAAATAATTGAGCCTTAAAGAAGAAATTC |
| | | TTTAAGTGGATGCTCTCAAACTCAGGGAAACCTAAA |
| | | TCTAGTTATAGACAAGGCAATCCTGAGCCAAGCCGA |
| | | AGTAGTAATTAGTAAGTTAAC |
| 123 | L6-4 | ACGGACTTAAATAATTGAGCCTTAAAGAAGAAATTC |
| | | TTTAAGTGGATGCTCTCAAACTCAGGGAAACCTAAA |
| | | TCTAGTTATAGACAAGGCAATCCTGAGCCAAGCCGA |
| | | AGTAGTAATTAGTAAGTTAACA |
| 124 | L6-5 | ACGGACTTAAATAATTGAGCCTTAAAGAAGAAATTC |
| | | TTTAAGTGGATGCTCTCAAACTCAGGGAAACCTAAA |
| | | TCTAGTTATAGACAAGGCAATCCTGAGCCAAGCCGA |
| | | AGTAGTAATTAGTAAGTTAACAA |
| 125 | L6-6 | ACGGACTTAAATAATTGAGCCTTAAAGAAGAAATTC |
| | | TTTAAGTGGATGCTCTCAAACTCAGGGAAACCTAAA |
| | | TCTAGTTATAGACAAGGCAATCCTGAGCCAAGCCGA |
| | | AGTAGTAATTAGTAAGTTAACAAT |
| 126 | L6-7 | ACGGACTTAAATAATTGAGCCTTAAAGAAGAAATTC |
| | | TTTAAGTGGATGCTCTCAAACTCAGGGAAACCTAAA |
| | | TCTAGTTATAGACAAGGCAATCCTGAGCCAAGCCGA |
| | | AGTAGTAATTAGTAAGTTAACAATA |
| 127 | L6-8 | ACGGACTTAAATAATTGAGCCTTAAAGAAGAAATTC |
| | | TTTAAGTGGATGCTCTCAAACTCAGGGAAACCTAAA |
| | | TCTAGTTATAGACAAGGCAATCCTGAGCCAAGCCGA |
| | | AGTAGTAATTAGTAAGTTAACAATAG |
| 128 | L6-9 | ACGGACTTAAATAATTGAGCCTTAAAGAAGAAATTC |
| | | TTTAAGTGGATGCTCTCAAACTCAGGGAAACCTAAA |
| | | TCTAGTTATAGACAAGGCAATCCTGAGCCAAGCCGA |
| | | AGTAGTAATTAGTAAGTTAACAATAGA |
| 129 | L8-1 | ACGGACTTAAATAATTGAGCCTTAAAGAAGAAATTC |
| | | TTTAAGTGGATGCTCTCAAACTCAGGGAAACCTAAA |
| | | TCTAGTTATAGACAAGGCAATCCTGAGCCAAGCCGA |
| | | AGTAGTAATTAGTAAGTTAACAATAGATGACTTACA |
| | | ACTAATCGGAAGGTGCAGAGACTCGACGGGAGCTA |
| | | CCCTAACGT |
| 130 | L8-2 | ACGGACTTAAATAATTGAGCCTTAAAGAAGAAATTC |
| | | TTTAAGTGGATGCTCTCAAACTCAGGGAAACCTAAA |
| | | TCTAGTTATAGACAAGGCAATCCTGAGCCAAGCCGA |
| | | AGTAGTAATTAGTAAGTTAACAATAGATGACTTACA |
| | | ACTAATCGGAAGGTGCAGAGACTCGACGGGAGCTA |
| | | CCCTAACGTC |
| 131 | L8-3 | ACGGACTTAAATAATTGAGCCTTAAAGAAGAAATTC |
| | | TTTAAGTGGATGCTCTCAAACTCAGGGAAACCTAAA |
| | | TCTAGTTATAGACAAGGCAATCCTGAGCCAAGCCGA |
| | | AGTAGTAATTAGTAAGTTAACAATAGATGACTTACA |
| | | ACTAATCGGAAGGTGCAGAGACTCGACGGGAGCTA |
| | | CCCTAACGTCA |
| 132 | L8-4 | ACGGACTTAAATAATTGAGCCTTAAAGAAGAAATTC |
| | | TTTAAGTGGATGCTCTCAAACTCAGGGAAACCTAAA |
| | | TCTAGTTATAGACAAGGCAATCCTGAGCCAAGCCGA |
| | | AGTAGTAATTAGTAAGTTAACAATAGATGACTTACA |
| | | ACTAATCGGAAGGTGCAGAGACTCGACGGGAGCTA |
| | | CCCTAACGTCAA |
| 133 | L8-5 | ACGGACTTAAATAATTGAGCCTTAAAGAAGAAATTC |
| | | TTTAAGTGGATGCTCTCAAACTCAGGGAAACCTAAA |
| | | TCTAGTTATAGACAAGGCAATCCTGAGCCAAGCCGA |
| | | AGTAGTAATTAGTAAGTTAACAATAGATGACTTACA |
| | | ACTAATCGGAAGGTGCAGAGACTCGACGGGAGCTA |
| | | CCCTAACGTCAAG |
| 134 | L9a-1 | ACGGACTTAAATAATTGAGCCTTAAAGAAGAAATTC |
| | | TTTAAGTGGATGCTCTCAAACTCAGGGAAACCTAAA |
| | | TCTAGTTATAGACAAGGCAATCCTGAGCCAAGCCGA |
| | | AGTAGTAATTAGTAAGTTAACAATAGATGACTTACA |
| | | ACTAATCGGAAGGTGCAGAGACTCGACGGGAGCTA |
| | | CCCTAACGTCAAGACGAGGGTAAAGAGAGAGTCCA |
| | | ATTCTCAAAGCC |
| 135 | L9a-2 | ACGGACTTAAATAATTGAGCCTTAAAGAAGAAATTC |
| | | TTTAAGTGGATGCTCTCAAACTCAGGGAAACCTAAA |
| | | TCTAGTTATAGACAAGGCAATCCTGAGCCAAGCCGA |
| | | AGTAGTAATTAGTAAGTTAACAATAGATGACTTACA |
| | | ACTAATCGGAAGGTGCAGAGACTCGACGGGAGCTA |
| | | CCCTAACGTCAAGACGAGGGTAAAGAGAGAGTCCA |
| | | ATTCTCAAAGCCA |
| 136 | L9a-3 | ACGGACTTAAATAATTGAGCCTTAAAGAAGAAATTC |
| | | TTTAAGTGGATGCTCTCAAACTCAGGGAAACCTAAA |
| | | TCTAGTTATAGACAAGGCAATCCTGAGCCAAGCCGA |
| | | AGTAGTAATTAGTAAGTTAACAATAGATGACTTACA |
| | | ACTAATCGGAAGGTGCAGAGACTCGACGGGAGCTA |
| | | CCCTAACGTCAAGACGAGGGTAAAGAGAGAGTCCA |
| | | ATTCTCAAAGCCAA |
| 137 | L9a-4 | ACGGACTTAAATAATTGAGCCTTAAAGAAGAAATTC |
| | | TTTAAGTGGATGCTCTCAAACTCAGGGAAACCTAAA |
| | | TCTAGTTATAGACAAGGCAATCCTGAGCCAAGCCGA |
| | | AGTAGTAATTAGTAAGTTAACAATAGATGACTTACA |
| | | ACTAATCGGAAGGTGCAGAGACTCGACGGGAGCTA |
| | | CCCTAACGTCAAGACGAGGGTAAAGAGAGAGTCCA |
| | | ATTCTCAAAGCCAAT |
| 138 | L9a-5 | ACGGACTTAAATAATTGAGCCTTAAAGAAGAAATTC |
| | | TTTAAGTGGATGCTCTCAAACTCAGGGAAACCTAAA |
| | | TCTAGTTATAGACAAGGCAATCCTGAGCCAAGCCGA |
| | | AGTAGTAATTAGTAAGTTAACAATAGATGACTTACA |
| | | ACTAATCGGAAGGTGCAGAGACTCGACGGGAGCTA |
| | | CCCTAACGTCAAGACGAGGGTAAAGAGAGAGTCCA |
| | | ATTCTCAAAGCCAATA |
| 139 | L9-1 | ACGGACTTAAATAATTGAGCCTTAAAGAAGAAATTC |
| | | TTTAAGTGGATGCTCTCAAACTCAGGGAAACCTAAA |
| | | TCTAGTTATAGACAAGGCAATCCTGAGCCAAGCCGA |
| | | AGTAGTAATTAGTAAGTTAACAATAGATGACTTACA |
| | | ACTAATCGGAAGGTGCAGAGACTCGACGGGAGCTA |
| | | CCCTAACGTCAAGACGAGGGTAAAGAGAGAGTCCA |
| | | ATTCTCAAAGCCAATAGGCAGTAGC |
| 140 | L9-2 | ACGGACTTAAATAATTGAGCCTTAAAGAAGAAATTC |
| | | TTTAAGTGGATGCTCTCAAACTCAGGGAAACCTAAA |
| | | TCTAGTTATAGACAAGGCAATCCTGAGCCAAGCCGA |
| | | AGTAGTAATTAGTAAGTTAACAATAGATGACTTACA |
| | | ACTAATCGGAAGGTGCAGAGACTCGACGGGAGCTA |
| | | CCCTAACGTCAAGACGAGGGTAAAGAGAGAGTCCA |
| | | ATTCTCAAAGCCAATAGGCAGTAGCG |
| 141 | L9-3 | ACGGACTTAAATAATTGAGCCTTAAAGAAGAAATTC |
| | | TTTAAGTGGATGCTCTCAAACTCAGGGAAACCTAAA |
| | | TCTAGTTATAGACAAGGCAATCCTGAGCCAAGCCGA |
| | | AGTAGTAATTAGTAAGTTAACAATAGATGACTTACA |
| | | ACTAATCGGAAGGTGCAGAGACTCGACGGGAGCTA |
| | | CCCTAACGTCAAGACGAGGGTAAAGAGAGAGTCCA |
| | | ATTCTCAAAGCCAATAGGCAGTAGCGA |
| 142 | L9-4 | ACGGACTTAAATAATTGAGCCTTAAAGAAGAAATTC |
| | | TTTAAGTGGATGCTCTCAAACTCAGGGAAACCTAAA |
| | | TCTAGTTATAGACAAGGCAATCCTGAGCCAAGCCGA |
| | | AGTAGTAATTAGTAAGTTAACAATAGATGACTTACA |
| | | ACTAATCGGAAGGTGCAGAGACTCGACGGGAGCTA |
| | | CCCTAACGTCAAGACGAGGGTAAAGAGAGAGTCCA |
| | | ATTCTCAAAGCCAATAGGCAGTAGCGAA |
| 143 | L9-5 | ACGGACTTAAATAATTGAGCCTTAAAGAAGAAATTC |
| | | TTTAAGTGGATGCTCTCAAACTCAGGGAAACCTAAA |
| | | TCTAGTTATAGACAAGGCAATCCTGAGCCAAGCCGA |
| | | AGTAGTAATTAGTAAGTTAACAATAGATGACTTACA |
| | | ACTAATCGGAAGGTGCAGAGACTCGACGGGAGCTA |
| | | CCCTAACGTCAAGACGAGGGTAAAGAGAGAGTCCA |
| | | ATTCTCAAAGCCAATAGGCAGTAGCGAAA |
| 144 | L9-6 | ACGGACTTAAATAATTGAGCCTTAAAGAAGAAATTC |
| | | TTTAAGTGGATGCTCTCAAACTCAGGGAAACCTAAA |
| | | TCTAGTTATAGACAAGGCAATCCTGAGCCAAGCCGA |
| | | AGTAGTAATTAGTAAGTTAACAATAGATGACTTACA |
| | | ACTAATCGGAAGGTGCAGAGACTCGACGGGAGCTA |
| | | CCCTAACGTCAAGACGAGGGTAAAGAGAGAGTCCA |
| | | ATTCTCAAAGCCAATAGGCAGTAGCGAAAG |
| 145 | L9-7 | ACGGACTTAAATAATTGAGCCTTAAAGAAGAAATTC |
| | | TTTAAGTGGATGCTCTCAAACTCAGGGAAACCTAAA |
| | | TCTAGTTATAGACAAGGCAATCCTGAGCCAAGCCGA |
| | | AGTAGTAATTAGTAAGTTAACAATAGATGACTTACA |
| | | ACTAATCGGAAGGTGCAGAGACTCGACGGGAGCTA |
| | | CCCTAACGTCAAGACGAGGGTAAAGAGAGAGTCCA |
| | | ATTCTCAAAGCCAATAGGCAGTAGCGAAAGCTGC |
| 146 | L9-8 | ACGGACTTAAATAATTGAGCCTTAAAGAAGAAATTC |
| | | TTTAAGTGGATGCTCTCAAACTCAGGGAAACCTAAA |
| | | TCTAGTTATAGACAAGGCAATCCTGAGCCAAGCCGA |
| | | AGTAGTAATTAGTAAGTTAACAATAGATGACTTACA |
| | | ACTAATCGGAAGGTGCAGAGACTCGACGGGAGCTA |
| | | CCCTAACGTCAAGACGAGGGTAAAGAGAGAGTCCA |
| | | ATTCTCAAAGCCAATAGGCAGTAGCGAAAGCTGCA |
| 147 | L9-9 | ACGGACTTAAATAATTGAGCCTTAAAGAAGAAATTC |
| | | TTTAAGTGGATGCTCTCAAACTCAGGGAAACCTAAA |
| | | TCTAGTTATAGACAAGGCAATCCTGAGCCAAGCCGA |
| | | AGTAGTAATTAGTAAGTTAACAATAGATGACTTACA |
| | | ACTAATCGGAAGGTGCAGAGACTCGACGGGAGCTA |
| | | CCCTAACGTCAAGACGAGGGTAAAGAGAGAGTCCA |
| | | ATTCTCAAAGCCAATAGGCAGTAGCGAAAGCTGCAA |
| 148 | L9a-6 | ACGGACTTAAATAATTGAGCCTTAAAGAAGAAATTC |
| | | TTTAAGTGGATGCTCTCAAACTCAGGGAAACCTAAA |
| | | TCTAGTTATAGACAAGGCAATCCTGAGCCAAGCCGA |
| | | AGTAGTAATTAGTAAGTTAACAATAGATGACTTACA |
| | | ACTAATCGGAAGGTGCAGAGACTCGACGGGAGCTA |
| | | CCCTAACGTCAAGACGAGGGTAAAGAGAGAGTCCA |
| | | ATTCTCAAAGCCAATAG |
| 149 | L9a-7 | ACGGACTTAAATAATTGAGCCTTAAAGAAGAAATTC |
| | | TTTAAGTGGATGCTCTCAAACTCAGGGAAACCTAAA |
| | | TCTAGTTATAGACAAGGCAATCCTGAGCCAAGCCGA |
| | | AGTAGTAATTAGTAAGTTAACAATAGATGACTTACA |
| | | ACTAATCGGAAGGTGCAGAGACTCGACGGGAGCTA |
| | | CCCTAACGTCAAGACGAGGGTAAAGAGAGAGTCCA |
| | | ATTCTCAAAGCCAATAGGC |
| 150 | L9a-8 | ACGGACTTAAATAATTGAGCCTTAAAGAAGAAATTC |
| | | TTTAAGTGGATGCTCTCAAACTCAGGGAAACCTAAA |
| | | TCTAGTTATAGACAAGGCAATCCTGAGCCAAGCCGA |
| | | AGTAGTAATTAGTAAGTTAACAATAGATGACTTACA |
| | | ACTAATCGGAAGGTGCAGAGACTCGACGGGAGCTA |
| | | CCCTAACGTCAAGACGAGGGTAAAGAGAGAGTCCA |
| | | ATTCTCAAAGCCAATAGGCA |

In some embodiments, a 3′ intron fragment is a fragment having a sequence listed in Table 3. In some embodiments, a construct containing a 3′ intron fragment listed in Table 3 will contain a corresponding 5′ intron fragment as listed in Table 2 (e.g., both representing fragments with the L9a-8 permutation site).

TABLE 4
Non-anabaena permutation site
5′ intron fragment sequences.

| SEQ | | |
|---|---|---|
| ID NO: | Intron | Sequence |
| 151 | Azop1 | tgcgccgatgaaggtgtagagactagacg |
| | | gcacccacctaaggcaaacgctatggtga |
| | | aggcatagtccagggagtggcgaaagtca |
| | | cacaaaccggaatccgt |
| 152 | Azop2 | ccgggcgtatggcaacgccgagccaagct |
| | | tcggcgcctgcgccgatgaaggtgtagag |
| | | actagacggcacccacctaaggcaaacgc |
| | | tatggtgaaggcatagtccagggagtggc |
| | | gaaagtcacacaaaccggaatccgt |
| 153 | Azop3 | acggcacccacctaaggcaaacgctatgg |
| | | tgaaggcatagtccagggagtggcgaaag |
| | | tcacacaaaccggaatccgt |
| 154 | Azop4 | acgctatggtgaaggcatagtccagggag |
| | | tggcgaaagtcacacaaaccggaatccgt |
| 155 | S795p1 | attaaagttatagaattatcagagaatga |
| | | tatagtccaagccttatggtaacatgagg |
| | | gacttgaccctggtag |
| 156 | Twortp1 | aagatgtaggcaatcctgagctaagctct |
| | | tagtaataagagaaagtgcaacgactatt |
| | | ccgataggaagtagggtcaagtgactcga |
| | | aatggggattacccttctagggtagtgat |
| | | atagtctgaacatatatggaaacatatag |
| | | aaggataggagtaacgaacctattcgtaa |
| | | cataattgaacttttagttat |
| 157 | Twortp2 | taataagagaaagtgcaacgactattccg |
| | | ataggaagtagggtcaagtgactcgaaat |
| | | ggggattacccttctagggtagtgatata |
| | | gtctgaacatatatggaaacatatagaag |
| | | gataggagtaacgaacctattcgtaacat |
| | | aattgaacttttagttat |
| 158 | Twortp3 | taggaagtagggtcaagtgactcgaaatg |
| | | gggattacccttctagggtagtgatatag |
| | | tctgaacatatatggaaacatatagaagg |
| | | ataggagtaacgaacctattcgtaacata |
| | | attgaacttttagttat |
| 159 | Twortp4 | ctagggtagtgatatagtctgaacatata |
| | | tggaaacatatagaaggataggagtaacg |
| | | aacctattcgtaacataattgaactttta |
| | | gttat |
| 160 | LSUp1 | agttaataaagatgatgaaatagtctgaa |
| | | ccattttgagaaaagtggaaataaaagaa |
| | | aatcttttatgataacataaattgaacag |
| | | gctaa |
| 161 | Phip1 | caaagactgatgatatagtccgacactcc |
| | | tagtaataggagaatacagaaaggatgaa |
| | | atcc |
| 162 | Nostoc | agtcgagggtaaagggagagtccaattct |
| | | caaagcctattggcagtagcgaaagctgc |
| | | gggagaatgaaaatccgt |
| 163 | Nostoc | agccgagggtaaagggagagtccaattct |
| | | caaagccaataggcagtagcgaaagctgc |
| | | gggagaatgaaaatccgt |
| 164 | Nodularia | agccgagggtaaagggagagtccaattct |
| | | caaagccgaaggttattaaaacctggcag |
| | | cagtgaaagctgcgggagaatgaaaatcc |
| | | gt |
| 165 | Pleurocapsa | agctgagggtaaagagagagtccaattct |
| | | caaagccagcagatggcagtagcgaaagc |
| | | tgcgggagaatgaaaatccgt |
| 166 | Planktothrix | agccgagggtaaagagagagtccaattct |
| | | caaagccaattggtagtagcgaaagctac |
| | | gggagaatgaaaatccgt |

In some embodiments, a 5′ intron fragment is a fragment having a sequence listed in Table 4. A construct containing a 5′ intron fragment listed in Table 4 will contain a corresponding 3′ intron fragment as listed in Table 5 (e.g., both representing fragments with the Azop1 intron).

TABLE 5
Non-anabaena permutation site
3′ intron fragment sequences.

| SEQ | | |
|---|---|---|
| ID NO: | Intron | Sequence |
| 167 | Azop1 | gcggactcatatttcgatgtgccttgcgc |
| | | cgggaaaccacgcaagggatggtgtcaaa |
| | | ttcggcgaaacctaagcgcccgcccgggc |
| | | gtatggcaacgccgagccaagcttcggcg |
| | | cc |
| 168 | Azop2 | gcggactcatatttcgatgtgccttgcgc |
| | | cgggaaaccacgcaagggatggtgtcaaa |
| | | ttcggcgaaacctaagcgcccgc |
| 169 | Azop3 | gcggactcatatttcgatgtgccttgcgc |
| | | cgggaaaccacgcaagggatggtgtcaaa |
| | | ttcggcgaaacctaagcgcccgcccgggc |
| | | gtatggcaacgccgagccaagcttcggcg |
| | | cctgcgccgatgaaggtgtagagactag |
| 170 | Azop4 | gcggactcatatttcgatgtgccttgcgc |
| | | cgggaaaccacgcaagggatggtgtcaaa |
| | | ttcggcgaaacctaagcgcccgcccgggc |
| | | gtatggcaacgccgagccaagcttcggcg |
| | | cctgcgccgatgaaggtgtagagactaga |
| | | cggcacccacctaaggcaa |
| 171 | S795p1 | aggattagatactacactaagtgtccccc |
| | | agactggtgacagtctggtgtgcatccag |
| | | ctatatcggtgaaaccccattggggtaat |
| | | accgagggaagctatattatatatatatt |
| | | aataaatagccccgtagagactatgtagg |
| | | taaggagatagaagatgataaaatcaaaa |
| | | tcatc |
| 172 | Twortp1 | actactgaaagcataaataattgtgcctt |
| | | tatacagtaatgtatatcgaaaaatcctc |
| | | taattcagggaacacctaaacaaact |
| 173 | Twortp2 | actactgaaagcataaataattgtgcctt |
| | | tatacagtaatgtatatcgaaaaatcctc |
| | | taattcagggaacacctaaacaaactaag |
| | | atgtaggcaatcctgagctaagctcttag |
| 174 | Twortp3 | actactgaaagcataaataattgtgcctt |
| | | tatacagtaatgtatatcgaaaaatcctc |
| | | taattcagggaacacctaaacaaactaag |
| | | atgtaggcaatcctgagctaagctcttag |
| | | taataagagaaagtgcaacgactattccg |
| | | a |
| 175 | Twortp4 | actactgaaagcataaataattgtgcctt |
| | | tatacagtaatgtatatcgaaaaatcctc |
| | | taattcagggaacacctaaacaaactaag |
| | | atgtaggcaatcctgagctaagctcttag |
| | | taataagagaaagtgcaacgactattccg |
| | | ataggaagtagggtcaagtgactcgaaat |
| | | ggggattaccctt |
| 176 | LSUp1 | cgctagggatttataactgtgagtcctcc |
| | | aatattataaaatgttggtaatatattgg |
| | | gtaaatttcaaagacaacttttctccacg |
| | | tcaggatatagtgtatttgaagcgaaact |
| | | tattttagcagtgaaaaagcaaataagga |
| | | cgttcaacgactaaaaggtgagtattgct |
| | | aacaataatccttttttttaatgcccaac |
| | | atctttattaact |
| 177 | Phip1 | gtgggtgcataaactatttcattgtgcac |
| | | attaaatctggtgaactcggtgaaaccct |
| | | aatggggcaataccgagccaagccatagg |
| | | gaggatatatgagaggcaagaagttaatt |
| | | cttgaggccactgagactggctgtatcat |
| | | ccctacgtcacacaaacttaatgccgatg |
| | | gttatttcagaaagaaaaccaatggcgtc |
| | | ttagagatgtatcacagaacggtgtggaa |
| | | ggagcataacggagacatacctgatggct |
| | | tcgagatagaccataagtgtcgcaatagg |
| | | gcttgctgtaatatagagcatttacagat |
| | | gcttgagggtacagcccacactgttaaga |
| | | ccaatcgtgaacgctacgcagacagaaag |
| | | gaaacagctagggaatactggctggagac |
| | | tggatgtaccggcctagcactcggtgaga |
| | | agtttggtgtgtcgttctcttctgcttgt |
| | | aagtggattagagaatggaaggcgtagag |
| | | actatccgaaaggagtagggccgagggtg |
| | | agactccctcgtaacccgaagcgccagac |
| | | agtcaact |
| 178 | Nostoc | acggacttaagtaattgagccttaaagaa |
| | | gaaattctttaagtggcagctctcaaact |
| | | cagggaaacctaaatctgttcacagacaa |
| | | ggcaatcctgagccaagccgaaagagtca |
| | | tgagtgctgagtagtgagtaaaataaaag |
| | | ctcacaactcagaggttgtaactctaagc |
| | | tagtcggaaggtgcagagactcgacggga |
| | | gctaccctaacgtaa |
| 179 | Nostoc | acggacttaaactgaattgagccttagag |
| | | aagaaattctttaagtgtcagctctcaaa |
| | | ctcagggaaacctaaatctgttgacagac |
| | | aaggcaatcctgagccaagccgagaactc |
| | | taagttattcggaaggtgcagagactcga |
| | | cgggagctaccctaacgtca |
| 180 | Nodularia | acggacttagaaaactgagccttgatcga |
| | | gaaatctttcaagtggaagctctcaaatt |
| | | cagggaaacctaaatctgtttacagatat |
| | | ggcaatcctgagccaagccgaaacaagtc |
| | | ctgagtgttaaagctcataactcatcgga |
| | | aggtgcagagactcgacgggagctaccct |
| | | aacgtta |
| 181 | Pleurocapsa | acggacttaaaaaaattgagccttggcag |
| | | agaaatctgtcatgcgaacgctctcaaat |
| | | tcagggaaacctaagtctggcaacagata |
| | | tggcaatcctgagccaagccttaatcaag |
| | | gaaaaaaacatttttaccttttaccttga |
| | | aaggaaggtgcagagactcaacgggagct |
| | | accctaacaggtca |
| 182 | Planktothrix | acggacttaaagataaattgagccttgag |
| | | gcgagaaatctctcaagtgtaagctgtca |
| | | aattcagggaaacctaaatctgtaaattc |
| | | agacaaggcaatcctgagccaagcctagg |
| | | ggtattagaaatgagggagtttccccaat |
| | | ctaagatcaatacctaggaaggtgcagag |
| | | actcgacgggagctaccctaacgtta |

In some embodiments, a 3′ intron fragment is a fragment having a sequence listed in Table 5. A construct containing a 3′ intron fragment listed in Table 5 will contain the corresponding 5′ intron fragment as listed in Table 4 (e.g., both representing fragments with the Azop1 intron).

TABLE 6
Spacer and Anabaena 5′
intron fragment sequences.

| SEQ | | |
|---|---|---|
| ID NO: | Spacer | Sequence |
| 183 | T25 L10 | agtatataagaaacaaaccacTAGATGAC |
| | | TTACAACTAATCGGAAGGTGCAGAGACTC |
| | | GACGGGAGCTACCCTAACGTCAAGACGAG |
| | | GGTAAAGAGAGAGTCCAATTCTCAAAGCC |
| | | AATAGGCAGTAGCGAAAGCTGCAAGAGAA |
| | | TGAAAATCCGTggctcgcagc |
| 184 | T25 L20 | ctgaaattatacttatactcaaacaaacc |
| | | acTAGATGACTTACAACTAATCGGAAGGT |
| | | GCAGAGACTCGACGGGAGCTACCCTAACG |
| | | TCAAGACGAGGGTAAAGAGAGAGTCCAAT |
| | | TCTCAAAGCCAATAGGCAGTAGCGAAAGC |
| | | TGCAAGAGAATGAAAATCCGTggctcgca |
| | | gc |
| 185 | T25 L30 (I80- | ctgaaattatacttatactcagtatatga |
| | 10) [Control] | caaacaaaccacTAGATGACTTACAACTA |
| | | ATCGGAAGGTGCAGAGACTCGACGGGAGC |
| | | TACCCTAACGTCAAGACGAGGGTAAAGAG |
| | | AGAGTCCAATTCTCAAAGCCAATAGGCAG |
| | | TAGCGAAAGCTGCAAGAGAATGAAAATCC |
| | | GTggctcgcagc |
| 186 | T25 L40 | catcaacaatatgaaattatacttatact |
| | | cagtatatgacaaacaaaccacTAGATGA |
| | | CTTACAACTAATCGGAAGGTGCAGAGACT |
| | | CGACGGGAGCTACCCTAACGTCAAGACGA |
| | | GGGTAAAGAGAGAGTCCAATTCTCAAAGC |
| | | CAATAGGCAGTAGCGAAAGCTGCAAGAGA |
| | | ATGAAAATCCGTggctcgcagc |
| 187 | T25 L50 | catcaacaatatgaaactatacttatact |
| | | cagtatatgaagcattatcgcaaacaaac |
| | | cacTAGATGACTTACAACTAATCGGAAGG |
| | | TGCAGAGACTCGACGGGAGCTACCCTAAC |
| | | GTCAAGACGAGGGTAAAGAGAGAGTCCAA |
| | | TTCTCAAAGCCAATAGGCAGTAGCGAAAG |
| | | CTGCAAGAGAATGAAAATCCGTggctcgc |
| | | agc |
| 188 | T50 L10 | tagcgtcagcaaacaaacaaaTAGATGAC |
| | | TTACAACTAATCGGAAGGTGCAGAGACTC |
| | | GACGGGAGCTACCCTAACGTCAAGACGAG |
| | | GGTAAAGAGAGAGTCCAATTCTCAAAGCC |
| | | AATAGGCAGTAGCGAAAGCTGCAAGAGAA |
| | | TGAAAATCCGTggctcgcagc |
| 189 | T50 L20 | atactcatactagcgtcagcaaacaaaca |
| | | aaTAGATGACTTACAACTAATCGGAAGGT |
| | | GCAGAGACTCGACGGGAGCTACCCTAACG |
| | | TCAAGACGAGGGTAAAGAGAGAGTCCAAT |
| | | TCTCAAAGCCAATAGGCAGTAGCGAAAGC |
| | | TGCAAGAGAATGAAAATCCGTggctcgca |
| | | gc |
| 190 | T50 L30 | gtgtgaagctatactcatactagcgtcag |
| | | caaacaaacaaaTAGATGACTTACAACTA |
| | | ATCGGAAGGTGCAGAGACTCGACGGGAGC |
| | | TACCCTAACGTCAAGACGAGGGTAAAGAG |
| | | AGAGTCCAATTCTCAAAGCCAATAGGCAG |
| | | TAGCGAAAGCTGCAAGAGAATGAAAATCC |
| | | GTggctcgcagc |
| 191 | T50 L40 | cctcacctgagtgtgaagctatactcata |
| | | ctagcgtcagcaaacaaacaaaTAGATGA |
| | | CTTACAACTAATCGGAAGGTGCAGAGACT |
| | | CGACGGGAGCTACCCTAACGTCAAGACGA |
| | | GGGTAAAGAGAGAGTCCAATTCTCAAAGC |
| | | CAATAGGCAGTAGCGAAAGCTGCAAGAGA |
| | | ATGAAAATCCGTggctcgcagc |
| 192 | T50 L50 | ccgaatgatgcctcacctgagtgtgaagc |
| | | tatactcatactagcgtcagcaaacaaac |
| | | aaaTAGATGACTTACAACTAATCGGAAGG |
| | | TGCAGAGACTCGACGGGAGCTACCCTAAC |
| | | GTCAAGACGAGGGTAAAGAGAGAGTCCAA |
| | | TTCTCAAAGCCAATAGGCAGTAGCGAAAG |
| | | CTGCAAGAGAATGAAAATCCGTggctcgc |
| | | agc |
| 193 | T75 L10 | cggtgcgagcaaacaaacaaaTAGATGAC |
| | | TTACAACTAATCGGAAGGTGCAGAGACTC |
| | | GACGGGAGCTACCCTAACGTCAAGACGAG |
| | | GGTAAAGAGAGAGTCCAATTCTCAAAGCC |
| | | AATAGGCAGTAGCGAAAGCTGCAAGAGAA |
| | | TGAAAATCCGTggctcgcagc |
| 194 | T75 L20 | cgctccgacccagtgcgagcaaacaaaca |
| | | aaTAGATGACTTACAACTAATCGGAAGGT |
| | | GCAGAGACTCGACGGGAGCTACCCTAACG |
| | | TCAAGACGAGGGTAAAGAGAGAGTCCAAT |
| | | TCTCAAAGCCAATAGGCAGTAGCGAAAGC |
| | | TGCAAGAGAATGAAAATCCGTggctcgca |
| | | gc |
| 195 | T25 L30 | ctgaaattatactAatactcagtatatga |
| | 1MM | caaacaaaccacTAGATGACTTACAACTA |
| | | ATCGGAAGGTGCAGAGACTCGACGGGAGC |
| | | TACCCTAACGTCAAGACGAGGGTAAAGAG |
| | | AGAGTCCAATTCTCAAAGCCAATAGGCAG |
| | | TAGCGAAAGCTGCAAGAGAATGAAAATCC |
| | | GTggctcgcagc |
| 196 | T25 L30 | ctgaaaAtatactAatactcaCtatatga |
| | 3MM | caaacaaaccacTAGATGACTTACAACTA |
| | | ATCGGAAGGTGCAGAGACTCGACGGGAGC |
| | | TACCCTAACGTCAAGACGAGGGTAAAGAG |
| | | AGAGTCCAATTCTCAAAGCCAATAGGCAG |
| | | TAGCGAAAGCTGCAAGAGAATGAAAATCC |
| | | GTggctcgcagc |
| 197 | T25 L30 | ctgaTaAtataGtAatactcaCtatatga |
| | 5MM | caaacaaaccacTAGATGACTTACAACTA |
| | | ATCGGAAGGTGCAGAGACTCGACGGGAGC |
| | | TACCCTAACGTCAAGACGAGGGTAAAGAG |
| | | AGAGTCCAATTCTCAAAGCCAATAGGCAG |
| | | TAGCGAAAGCTGCAAGAGAATGAAAATCC |
| | | GTggctcgcagc |
| 198 | T25 L30 | ctgaTaAtaAaGtAatacAcaCtataAga |
| | 8MM | caaacaaaccacTAGATGACTTACAACTA |
| | | ATCGGAAGGTGCAGAGACTCGACGGGAGC |
| | | TACCCTAACGTCAAGACGAGGGTAAAGAG |
| | | AGAGTCCAATTCTCAAAGCCAATAGGCAG |
| | | TAGCGAAAGCTGCAAGAGAATGAAAATCC |
| | | GTggctcgcagc |
| 199 | T25 L30 | ctgaaattatacttatactctctaagtta |
| | OffTarget 10 | caaacaaaccacTAGATGACTTACAACTA |
| | | ATCGGAAGGTGCAGAGACTCGACGGGAGC |
| | | TACCCTAACGTCAAGACGAGGGTAAAGAG |
| | | AGAGTCCAATTCTCAAAGCCAATAGGCAG |
| | | TAGCGAAAGCTGCAAGAGAATGAAAATCC |
| | | GTggctcgcagc |
| 200 | T25 L30 | ctgaaattatgtgtgttacAtctaagtta |
| | OffTarget 20 | caaacaaaccacTAGATGACTTACAACTA |
| | | ATCGGAAGGTGCAGAGACTCGACGGGAGC |
| | | TACCCTAACGTCAAGACGAGGGTAAAGAG |
| | | AGAGTCCAATTCTCAAAGCCAATAGGCAG |
| | | TAGCGAAAGCTGCAAGAGAATGAAAATCC |
| | | GTggctcgcagc |
| 201 | T25 L30 | gttgatcggtgtgtgttacAtctaagtta |
| | OffTarget 30 | caaacaaaccacTAGATGACTTACAACTA |
| | | ATCGGAAGGTGCAGAGACTCGACGGGAGC |
| | | TACCCTAACGTCAAGACGAGGGTAAAGAG |
| | | AGAGTCCAATTCTCAAAGCCAATAGGCAG |
| | | TAGCGAAAGCTGCAAGAGAATGAAAATCC |
| | | GTggctcgcagc |
| 202 | T25 L30 I25- | ctgaaattatacttatactcagtatatga |
| | 10 | caaacaaaccacTAGATGACTTACAACTA |
| | | ATCGGAAGGTGCAGAGACTCGACGGGAGC |
| | | TACCCTAACGTCAAGACGAGGGTAAAGAG |
| | | AGAGTCCAATTCTCAAAGCCAATAGGCAG |
| | | TAGCGAAAGCTGCAAGAGAATGAAAATCC |
| | | GTgattaaacag |
| 203 | T25 L30 I25- | ctgaaattatacttatactcagtatatga |
| | 20 | caaacaaaccacTAGATGACTTACAACTA |
| | | ATCGGAAGGTGCAGAGACTCGACGGGAGC |
| | | TACCCTAACGTCAAGACGAGGGTAAAGAG |
| | | AGAGTCCAATTCTCAAAGCCAATAGGCAG |
| | | TAGCGAAAGCTGCAAGAGAATGAAAATCC |
| | | GTgattcacaatataaattacg |
| 204 | T25 L30 I50- | ctgaaattatacttatactcagtatatga |
| | 10 | caaacaaaccacTAGATGACTTACAACTA |
| | | ATCGGAAGGTGCAGAGACTCGACGGGAGC |
| | | TACCCTAACGTCAAGACGAGGGTAAAGAG |
| | | AGAGTCCAATTCTCAAAGCCAATAGGCAG |
| | | TAGCGAAAGCTGCAAGAGAATGAAAATCC |
| | | GTggatcatagc |
| 205 | T25 L30 I50- | ctgaaattatacttatactcagtatatga |
| | 20 | caaacaaaccacTAGATGACTTACAACTA |
| | | ATCGGAAGGTGCAGAGACTCGACGGGAGC |
| | | TACCCTAACGTCAAGACGAGGGTAAAGAG |
| | | AGAGTCCAATTCTCAAAGCCAATAGGCAG |
| | | TAGCGAAAGCTGCAAGAGAATGAAAATCC |
| | | GTggatcgcagcataatatccg |
| 206 | T25 L30 I80- | ctgaaattatacttatactcagtatatga |
| | 20 | caaacaaaccacTAGATGACTTACAACTA |
| | | ATCGGAAGGTGCAGAGACTCGACGGGAGC |
| | | TACCCTAACGTCAAGACGAGGGTAAAGAG |
| | | AGAGTCCAATTCTCAAAGCCAATAGGCAG |
| | | TAGCGAAAGCTGCAAGAGAATGAAAATCC |
| | | GTggctcgcagcgcgcctaccg |
| 207 | T25 L30 I80- | ctgaaattatacttatactcagtatatga |
| | 20x2 | caaacaaaccacTAGATGACTTACAACTA |
| | | ATCGGAAGGTGCAGAGACTCGACGGGAGC |
| | | TACCCTAACGTCAAGACGAGGGTAAAGAG |
| | | AGAGTCCAATTCTCAAAGCCAATAGGCAG |
| | | TAGCGAAAGCTGCAAGAGAATGAAAATCC |
| | | GTggctcgcagcgcgcctaccgaaagccg |
| | | gcgtcgacgttagcgc |
| 208 | T25 L30 I50- | ctgaaattatacttatactcagtatatga |
| | 20x2 | caaacaaaccacTAGATGACTTACAACTA |
| | | ATCGGAAGGTGCAGAGACTCGACGGGAGC |
| | | TACCCTAACGTCAAGACGAGGGTAAAGAG |
| | | AGAGTCCAATTCTCAAAGCCAATAGGCAG |
| | | TAGCGAAAGCTGCAAGAGAATGAAAATCC |
| | | GTggatcgcagcataatatccgaaacgag |
| | | gatacaagtgacatgc |
| 209 | T25 L30 I25- | ctgaaattatacttatactcagtatatga |
| | 20x2 | caaacaaaccacTAGATGACTTACAACTA |
| | | ATCGGAAGGTGCAGAGACTCGACGGGAGC |
| | | TACCCTAACGTCAAGACGAGGGTAAAGAG |
| | | AGAGTCCAATTCTCAAAGCCAATAGGCAG |
| | | TAGCGAAAGCTGCAAGAGAATGAAAATCC |
| | | GTgattcacaatctaaattacgaaacgat |
| | | aaatgataactctaac |
| 210 | T0 L0 | aaacaaaccacTAGATGACTTACAACTAA |
| | | TCGGAAGGTGCAGAGACTCGACGGGAGCT |
| | | ACCCTAACGTCAAGACGAGGGTAAAGAGA |
| | | GAGTCCAATTCTCAAAGCCAATAGGCAGT |
| | | AGCGAAAGCTGCAAGAGAATGAAAATCCG |
| | | Tggctcgcagc |
| 211 | T100 L5 | cgggcaaacaaacaaaTAGATGACTTACA |
| | | ACTAATCGGAAGGTGCAGAGACTCGACGG |
| | | GAGCTACCCTAACGTCAAGACGAGGGTAA |
| | | AGAGAGAGTCCAATTCTCAAAGCCAATAG |
| | | GCAGTAGCGAAAGCTGCAAGAGAATGAAA |
| | | ATCCGTggctcgcagc |
| 212 | T75 L30 | cgctccgacgagcttccggccagtgcgag |
| | | caaacaaacaaaTAGATGACTTACAACTA |
| | | ATCGGAAGGTGCAGAGACTCGACGGGAGC |
| | | TACCCTAACGTCAAGACGAGGGTAAAGAG |
| | | AGAGTCCAATTCTCAAAGCCAATAGGCAG |
| | | TAGCGAAAGCTGCAAGAGAATGAAAATCC |
| | | GTggctcgcagc |
| 213 | T0 L0a | aaacaaaccacGGCAGTAGCGAAAGCTGC |
| | | AAGAGAATGAAAATCCGTggctcgcagc |
| 214 | T25 L10a | agtatataagaaacaaaccacGGCAGTAG |
| | | CGAAAGCTGCAAGAGAATGAAAATCCGTg |
| | | gctcgcagc |
| 215 | T25 L20a | ctgaaattatacttatactcaaacaaacc |
| | | acGGCAGTAGCGAAAGCTGCAAGAGAATG |
| | | AAAATCCGTggctgcagc |
| 216 | T25 L30a | ctgaaattatacttatactcagtatatga |
| | (I80-10) | caaacaaaccacGGCAGTAGCGAAAGCTG |
| | [Control] | CAAGAGAATGAAAATCCGTggctcgcagc |
| 217 | T50 L10a | tagcgtcagcaaacaaacaaaGGCAGTAG |
| | | CGAAAGCTGCAAGAGAATGAAAATCCGTg |
| | | gctcgcagc |
| 218 | T50 L20a | atactcatactagcgtcagcaaacaaaca |
| | | aaGGCAGTAGCGAAAGCTGCAAGAGAATG |
| | | AAAATCCGTggctcgcagc |
| 219 | T50 L30a | gtgtgaagctatactcatactagcgtcag |
| | | caaacaaacaaaGGCAGTAGCGAAAGCTG |
| | | CAAGAGAATGAAAATCCGTggctcgcagc |
| 220 | T75 L10a | cggtgcgagcaaacaaacaaaGGCAGTAG |
| | | CGAAAGCTGCAAGAGAATGAAAATCCGTg |
| | | gctcgcagc |
| 221 | T75 L20a | cgctccgacccagtgcgagcaaacaaaca |
| | | aaGGCAGTAGCGAAAGCTGCAAGAGAATG |
| | | AAAATCCGTggctcgcagc |
| 222 | T75 L30a | cgctccgacgagcttccggccagtgcgag |
| | | caaacaaacaaaGGCAGTAGCGAAAGCTG |
| | | CAAGAGAATGAAAATCCGTggctcgcagc |
| 223 | T0 L0b | aaacaaaccacAAGACGAGGGTAAAGAGA |
| | | GAGTCCAATTCTCAAAGCCAATAGGCAGT |
| | | AGCGAAAGCTGCAAGAGAATGAAAATCCG |
| | | Tggctcgcagc |
| 224 | T25 L10b | agtatataagaaacaaaccacAAGACGAG |
| | | GGTAAAGAGAGAGTCCAATTCTCAAAGCC |
| | | AATAGGCAGTAGCGAAAGCTGCAAGAGAA |
| | | TGAAAATCCGTggctcgcagc |
| 225 | T25 L20b | ctgaaattatacttatactcaaacaaacc |
| | | acAAGACGAGGGTAAAGAGAGAGTCCAAT |
| | | TCTCAAAGCCAATAGGCAGTAGCGAAAGC |
| | | TGCAAGAGAATGAAAATCCGTggctcgca |
| | | gc |
| 226 | T25 L30b | ctgaaattatacttatactcagtatatga |
| | (I80-10) | caaacaaaccacAAGACGAGGGTAAAGAG |
| | [Control] | AGAGTCCAATTCTCAAAGCCAATAGGCAG |
| | | TAGCGAAAGCTGCAAGAGAATGAAAATCC |
| | | GTggctcgcagc |
| 227 | T50 L10b | tagcgtcagcaaacaaacaaaAAGACGAG |
| | | GGTAAAGAGAGAGTCCAATTCTCAAAGCC |
| | | AATAGGCAGTAGCGAAAGCTGCAAGAGAA |
| | | TGAAAATCCGTggctcgcagc |
| 228 | T50 L20b | atactcatactagcgtcagcaaacaaaca |
| | | aaAAGACGAGGGTAAAGAGAGAGTCCAAT |
| | | TCTCAAAGCCAATAGGCAGTAGCGAAAGC |
| | | TGCAAGAGAATGAAAATCCGTggctcgca |
| | | gc |
| 229 | T50 L30b | gtgtgaagctatactcatactagcgtcag |
| | | caaacaaacaaaAAGACGAGGGTAAAGAG |
| | | AGAGTCCAATTCTCAAAGCCAATAGGCAG |
| | | TAGCGAAAGCTGCAAGAGAATGAAAATCC |
| | | GTggctcgcagc |
| 230 | T75 L10b | cggtgcgagcaaacaaacaaaAAGACGAG |
| | | GGTAAAGAGAGAGTCCAATTCTCAAAGCC |
| | | AATAGGCAGTAGCGAAAGCTGCAAGAGAA |
| | | TGAAAATCCGTggctcgcagc |
| 231 | T75 L20b | cgctccgacccagtgcgagcaaacaaaca |
| | | aaAAGACGAGGGTAAAGAGAGAGTCCAAT |
| | | TCTCAAAGCCAATAGGCAGTAGCGAAAGC |
| | | TGCAAGAGAATGAAAATCCGTggctcgca |
| | | gc |
| 232 | T75 L30b | cgctccgacgagcttccggccagtgcgag |
| | | caaacaaacaaaAAGACGAGGGTAAAGAG |
| | | AGAGTCCAATTCTCAAAGCCAATAGGCAG |
| | | TAGCGAAAGCTGCAAGAGAATGAAAATCC |
| | | GTggctcgcagc |
| 233 | T25 L30 I0-0 | ctgaaattatacttatactcagtatatga |
| | | caaacaaaccacTAGATGACTTACAACTA |
| | | ATCGGAAGGTGCAGAGACTCGACGGGAGC |
| | | TACCCTAACGTCAAGACGAGGGTAAAGAG |
| | | AGAGTCCAATTCTCAAAGCCAATAGGCAG |
| | | TAGCGAAAGCTGCAAGAGAATGAAAATCC |
| | | GT |
| 234 | T25 L30a I0-0 | ctgaaattatacttatactcagtatatga |
| | | caaacaaaccacGGCAGTAGCGAAAGCTG |
| | | CAAGAGAATGAAAATCCGT |
| 235 | T25 L30a I25- | ctgaaattatacttatactcagtatatga |
| | 10 | caaacaaaccacGGCAGTAGCGAAAGCTG |
| | | CAAGAGAATGAAAATCCGTgattaaacag |
| 236 | T25 L30a I25- | ctgaaattatacttatactcagtatatga |
| | 20 | caaacaaaccacGGCAGTAGCGAAAGCTG |
| | | CAAGAGAATGAAAATCCGTgattcacaat |
| | | ataaattacg |
| 237 | T25 L30a I50- | ctgaaattatacttatactcagtatatga |
| | 10 | caaacaaaccacGGCAGTAGCGAAAGCTG |
| | | CAAGAGAATGAAAATCCGTggatcatagc |
| 238 | T25 L30a I50- | ctgaaattatacttatactcagtatatga |
| | 20 | caaacaaaccacGGCAGTAGCGAAAGCTG |
| | | CAAGAGAATGAAAATCCGTggatcgcagc |
| | | ataatatccg |
| 239 | T25 L30a I80- | ctgaaattatacttatactcagtatatga |
| | 20 | caaacaaaccacGGCAGTAGCGAAAGCTG |
| | | CAAGAGAATGAAAATCCGTggctcgcagc |
| | | gcgcctaccg |
| 240 | T25 L30b I0- | ctgaaattatacttatactcagtatatga |
| | 0 | caaacaaaccacAAGACGAGGGTAAAGAG |
| | | AGAGTCCAATTCTCAAAGCCAATAGGCAG |
| | | TAGCGAAAGCTGCAAGAGAATGAAAATCC |
| | | GT |
| 241 | T25 L30b | ctgaaattatacttatactcagtatatga |
| | I25-10 | caaacaaaccacAAGACGAGGGTAAAGAG |
| | | AGAGTCCAATTCTCAAAGCCAATAGGCAG |
| | | TAGCGAAAGCTGCAAGAGAATGAAAATCC |
| | | GTgattaaaca |
| 242 | T25 L30b | ctgaaattatacttatactcagtatatga |
| | I25-20 | caaacaaaccacAAGACGAGGGTAAAGAG |
| | | AGAGTCCAATTCTCAAAGCCAATAGGCAG |
| | | TAGCGAAAGCTGCAAGAGAATGAAAATCC |
| | | GTgattcacaatataaattacg |
| 243 | T25 L30b | ctgaaattatacttatactcagtatatga |
| | I50-10 | caaacaaaccacAAGACGAGGGTAAAGAG |
| | | AGAGTCCAATTCTCAAAGCCAATAGGCAG |
| | | TAGCGAAAGCTGCAAGAGAATGAAAATCC |
| | | GTggatcatagc |
| 244 | T25 L30b | ctgaaattatacttatactcagtatatga |
| | I50-20 | caaacaaaccacAAGACGAGGGTAAAGAG |
| | | AGAGTCCAATTCTCAAAGCCAATAGGCAG |
| | | TAGCGAAAGCTGCAAGAGAATGAAAATCC |
| | | GTggatcgcagcataatatccg |
| 245 | T25 L30b | ctgaaattatacttatactcagtatatga |
| | I80-20 | caaacaaaccacAAGACGAGGGTAAAGAG |
| | | AGAGTCCAATTCTCAAAGCCAATAGGCAG |
| | | TAGCGAAAGCTGCAAGAGAATGAAAATCC |
| | | GTggctcgcagcgcgcctaccg |

In some embodiments, a spacer and 5′ intron fragment are spacers and fragments having sequences as listed in Table 6.

TABLE 7
Spacer and Anabaena 3′ intron fragment sequences.

| SEQ | | |
|---|---|---|
| ID NO: | Spacer | Sequence |
| 246 | T25 L10 | gctgcgagccACGGACTTAAATAATTGAGCCTTAAAGAA |
| | | GAAATTCTTTAAGTGGATGCTCTCAAACTCAGGGAA |
| | | ACCTAAATCTAGTTATAGACAAGGCAATCCTGAGCC |
| | | AAGCCGAAGTAGTAATTAGTAAGTTAACAAcacaaacac |
| | | aacttatatact |
| 247 | T25 L20 | gctgcgagccACGGACTTAAATAATTGAGCCTTAAAGAA |
| | | GAAATTCTTTAAGTGGATGCTCTCAAACTCAGGGAA |
| | | ACCTAAATCTAGTTATAGACAAGGCAATCCTGAGCC |
| | | AAGCCGAAGTAGTAATTAGTAAGTTAACAAcacaaacac |
| | | aagagtataagtataatttcag |
| 248 | T25 L30 (I80- | gctgcgagccACGGACTTAAATAATTGAGCCTTAAAGAA |
| | 10) [Control] | GAAATTCTTTAAGTGGATGCTCTCAAACTCAGGGAA |
| | | ACCTAAATCTAGTTATAGACAAGGCAATCCTGAGCC |
| | | AAGCCGAAGTAGTAATTAGTAAGTTAACAAcacaaacac |
| | | aagtcatatactgagtataagtataatttcag |
| 249 | T25 L40 | gctgcgagccACGGACTTAAATAATTGAGCCTTAAAGAA |
| | | GAAATTCTTTAAGTGGATGCTCTCAAACTCAGGGAA |
| | | ACCTAAATCTAGTTATAGACAAGGCAATCCTGAGCC |
| | | AAGCCGAAGTAGTAATTAGTAAGTTAACAAcacaaacac |
| | | aagtcatatactgagtataagtataatttcatattgttgatg |
| 250 | T25 L50 | gctgcgagccACGGACTTAAATAATTGAGCCTTAAAGAA |
| | | GAAATTCTTTAAGTGGATGCTCTCAAACTCAGGGAA |
| | | ACCTAAATCTAGTTATAGACAAGGCAATCCTGAGCC |
| | | AAGCCGAAGTAGTAATTAGTAAGTTAACAAcacaaacac |
| | | aagcgataatgcttcatatactgagtataagtatagtttcatattgttgatg |
| 251 | T50 L10 | gctgcgagccACGGACTTAAATAATTGAGCCTTAAAGAA |
| | | GAAATTCTTTAAGTGGATGCTCTCAAACTCAGGGAA |
| | | ACCTAAATCTAGTTATAGACAAGGCAATCCTGAGCC |
| | | AAGCCGAAGTAGTAATTAGTAAGTTAACAAaacaaaaac |
| | | aagctgacgcta |
| 252 | T50 L20 | gctgcgagccACGGACTTAAATAATTGAGCCTTAAAGAA |
| | | GAAATTCTTTAAGTGGATGCTCTCAAACTCAGGGAA |
| | | ACCTAAATCTAGTTATAGACAAGGCAATCCTGAGCC |
| | | AAGCCGAAGTAGTAATTAGTAAGTTAACAAaacaaaaac |
| | | aagctgacgctagtatgagtat |
| 253 | T50 L30 | gctgcgagccACGGACTTAAATAATTGAGCCTTAAAGAA |
| | | GAAATTCTTTAAGTGGATGCTCTCAAACTCAGGGAA |
| | | ACCTAAATCTAGTTATAGACAAGGCAATCCTGAGCC |
| | | AAGCCGAAGTAGTAATTAGTAAGTTAACAAaacaaaaac |
| | | aagctgacgctagtatgagtatagcttcacac |
| 254 | T50 L40 | gctgcgagccACGGACTTAAATAATTGAGCCTTAAAGAA |
| | | GAAATTCTTTAAGTGGATGCTCTCAAACTCAGGGAA |
| | | ACCTAAATCTAGTTATAGACAAGGCAATCCTGAGCC |
| | | AAGCCGAAGTAGTAATTAGTAAGTTAACAAaacaaaaac |
| | | aagctgacgctagtatgagtatagcttcacactcaggtgagg |
| 255 | T50 L50 | gctgcgagccACGGACTTAAATAATTGAGCCTTAAAGAA |
| | | GAAATTCTTTAAGTGGATGCTCTCAAACTCAGGGAA |
| | | ACCTAAATCTAGTTATAGACAAGGCAATCCTGAGCC |
| | | AAGCCGAAGTAGTAATTAGTAAGTTAACAAaacaaaaac |
| | | aagctgacgctagtatgagtatagcttcacactcaggtgaggcatcattcgg |
| 256 | T75 L10 | gctgcgagccACGGACTTAAATAATTGAGCCTTAAAGAA |
| | | GAAATTCTTTAAGTGGATGCTCTCAAACTCAGGGAA |
| | | ACCTAAATCTAGTTATAGACAAGGCAATCCTGAGCC |
| | | AAGCCGAAGTAGTAATTAGTAAGTTAACAAaacaaaaac |
| | | aagctcgcaccg |
| 257 | T75 L20 | gctgcgagccACGGACTTAAATAATTGAGCCTTAAAGAA |
| | | GAAATTCTTTAAGTGGATGCTCTCAAACTCAGGGAA |
| | | ACCTAAATCTAGTTATAGACAAGGCAATCCTGAGCC |
| | | AAGCCGAAGTAGTAATTAGTAAGTTAACAAaacaaaaac |
| | | aagctcgcactgggtcggagcg |
| 258 | T25 L30 | gctgcgagccACGGACTTAAATAATTGAGCCTTAAAGAA |
| | 1MM | GAAATTCTTTAAGTGGATGCTCTCAAACTCAGGGAA |
| | | ACCTAAATCTAGTTATAGACAAGGCAATCCTGAGCC |
| | | AAGCCGAAGTAGTAATTAGTAAGTTAACAAcacaaacac |
| | | aagtcatatactgagtataagtataatttcag |
| 259 | T25 L30 | gctgcgagccACGGACTTAAATAATTGAGCCTTAAAGAA |
| | 3MM | GAAATTCTTTAAGTGGATGCTCTCAAACTCAGGGAA |
| | | ACCTAAATCTAGTTATAGACAAGGCAATCCTGAGCC |
| | | AAGCCGAAGTAGTAATTAGTAAGTTAACAAcacaaacac |
| | | aagtcatatactgagtataagtataatttcag |
| 260 | T25 L30 | gctgcgagccACGGACTTAAATAATTGAGCCTTAAAGAA |
| | 5MM | GAAATTCTTTAAGTGGATGCTCTCAAACTCAGGGAA |
| | | ACCTAAATCTAGTTATAGACAAGGCAATCCTGAGCC |
| | | AAGCCGAAGTAGTAATTAGTAAGTTAACAAcacaaacac |
| | | aagtcatatactgagtataagtataatttcag |
| 261 | T25 L30 | gctgcgagccACGGACTTAAATAATTGAGCCTTAAAGAA |
| | 8MM | GAAATTCTTTAAGTGGATGCTCTCAAACTCAGGGAA |
| | | ACCTAAATCTAGTTATAGACAAGGCAATCCTGAGCC |
| | | AAGCCGAAGTAGTAATTAGTAAGTTAACAAcacaaacac |
| | | aagtcatatactgagtataagtataatttcag |
| 262 | T25 L30 | gctgcgagccACGGACTTAAATAATTGAGCCTTAAAGAA |
| | OffTarget 10 | GAAATTCTTTAAGTGGATGCTCTCAAACTCAGGGAA |
| | | ACCTAAATCTAGTTATAGACAAGGCAATCCTGAGCC |
| | | AAGCCGAAGTAGTAATTAGTAAGTTAACAAcacaaacac |
| | | aagtaacttagagagtataagtataatttcag |
| 263 | T25 L30 | gctgcgagccACGGACTTAAATAATTGAGCCTTAAAGAA |
| | OffTarget 20 | GAAATTCTTTAAGTGGATGCTCTCAAACTCAGGGAA |
| | | ACCTAAATCTAGTTATAGACAAGGCAATCCTGAGCC |
| | | AAGCCGAAGTAGTAATTAGTAAGTTAACAAcacaaacac |
| | | aagtaacttagaTgtaacacacataatttcag |
| 264 | T25 L30 | gctgcgagccACGGACTTAAATAATTGAGCCTTAAAGAA |
| | OffTarget 30 | GAAATTCTTTAAGTGGATGCTCTCAAACTCAGGGAA |
| | | ACCTAAATCTAGTTATAGACAAGGCAATCCTGAGCC |
| | | AAGCCGAAGTAGTAATTAGTAAGTTAACAAcacaaacac |
| | | aagtaacttagaTgtaacacacaccgatcaac |
| 265 | T25 L30 I25- | ctgtttaatcACGGACTTAAATAATTGAGCCTTAAAGAAG |
| | 10 | AAATTCTTTAAGTGGATGCTCTCAAACTCAGGGAAA |
| | | CCTAAATCTAGTTATAGACAAGGCAATCCTGAGCCA |
| | | AGCCGAAGTAGTAATTAGTAAGTTAACAAcacaaacacaa |
| | | gtcatatactgagtataagtataatttcag |
| 266 | T25 L30 I25- | cgtaatttatattgtgaatcACGGACTTAAATAATTGAGCCTTAA |
| | 20 | AGAAGAAATTCTTTAAGTGGATGCTCTCAAACTCAG |
| | | GGAAACCTAAATCTAGTTATAGACAAGGCAATCCTG |
| | | AGCCAAGCCGAAGTAGTAATTAGTAAGTTAACAAcac |
| | | aaacacaagtcatatactgagtataagtataatttcag |
| 267 | T25 L30 I50- | gctatgatccACGGACTTAAATAATTGAGCCTTAAAGAAG |
| | 10 | AAATTCTTTAAGTGGATGCTCTCAAACTCAGGGAAA |
| | | CCTAAATCTAGTTATAGACAAGGCAATCCTGAGCCA |
| | | AGCCGAAGTAGTAATTAGTAAGTTAACAAcacaaacacaa |
| | | gtcatatactgagtataagtataatttcag |
| 268 | T25 L30 I50- | cggatattatgctgcgatccACGGACTTAAATAATTGAGCCTTA |
| | 20 | AAGAAGAAATTCTTTAAGTGGATGCTCTCAAACTCA |
| | | GGGAAACCTAAATCTAGTTATAGACAAGGCAATCCT |
| | | GAGCCAAGCCGAAGTAGTAATTAGTAAGTTAACAAc |
| | | acaaacacaagtcatatactgagtataagtataatttcag |
| 269 | T25 L30 I80- | cggtaggcgcgctgcgagccACGGACTTAAATAATTGAGCCTT |
| | 20 | AAAGAAGAAATTCTTTAAGTGGATGCTCTCAAACTC |
| | | AGGGAAACCTAAATCTAGTTATAGACAAGGCAATCC |
| | | TGAGCCAAGCCGAAGTAGTAATTAGTAAGTTAACAA |
| | | cacaaacacaagtcatatactgagtataagtataatttcag |
| 270 | T25 L30 I80- | gcgctaacgtcgacgccggcaaacggtaggcgcgctgcgagccACGGACTT |
| | 20x2 | AAATAATTGAGCCTTAAAGAAGAAATTCTTTAAGTG |
| | | GATGCTCTCAAACTCAGGGAAACCTAAATCTAGTTA |
| | | TAGACAAGGCAATCCTGAGCCAAGCCGAAGTAGTA |
| | | ATTAGTAAGTTAACAAcacaaacacaagtcatatactgagtataagtata |
| | | atttcag |
| 271 | T25 L30 I50- | gcatgtcacttgtatcctcgaaacggatattatgctgcgatccACGGACTTAA |
| | 20x2 | ATAATTGAGCCTTAAAGAAGAAATTCTTTAAGTGGA |
| | | TGCTCTCAAACTCAGGGAAACCTAAATCTAGTTATA |
| | | GACAAGGCAATCCTGAGCCAAGCCGAAGTAGTAATT |
| | | AGTAAGTTAACAAcacaaacacaagtcatatactgagtataagtataatttc |
| | | ag |
| 272 | T25 L30 I25- | gttagagttatcatttatcgaaacgtaatttagattgtgaatcACGGACTTAAAT |
| | 20x2 | AATTGAGCCTTAAAGAAGAAATTCTTTAAGTGGATG |
| | | CTCTCAAACTCAGGGAAACCTAAATCTAGTTATAGA |
| | | CAAGGCAATCCTGAGCCAAGCCGAAGTAGTAATTAG |
| | | TAAGTTAACAAcacaaacacaagtcatatactgagtataagtataatttcag |
| 273 | T0 L0 | gctgcgagccACGGACTTAAATAATTGAGCCTTAAAGAA |
| | | GAAATTCTTTAAGTGGATGCTCTCAAACTCAGGGAA |
| | | ACCTAAATCTAGTTATAGACAAGGCAATCCTGAGCC |
| | | AAGCCGAAGTAGTAATTAGTAAGTTAACAAcacaaacac |
| | | aa |
| 274 | T100 L5 | gctgcgagccACGGACTTAAATAATTGAGCCTTAAAGAA |
| | | GAAATTCTTTAAGTGGATGCTCTCAAACTCAGGGAA |
| | | ACCTAAATCTAGTTATAGACAAGGCAATCCTGAGCC |
| | | AAGCCGAAGTAGTAATTAGTAAGTTAACAAaacaaaaac |
| | | aagcccg |
| 275 | T75 L30 | gctgcgagccACGGACTTAAATAATTGAGCCTTAAAGAA |
| | | GAAATTCTTTAAGTGGATGCTCTCAAACTCAGGGAA |
| | | ACCTAAATCTAGTTATAGACAAGGCAATCCTGAGCC |
| | | AAGCCGAAGTAGTAATTAGTAAGTTAACAAaacaaaaac |
| | | aagctcgcactggccggaagctcgtcggagcg |
| 276 | T0 L0a | gctgcgagccACGGACTTAAATAATTGAGCCTTAAAGAA |
| | | GAAATTCTTTAAGTGGATGCTCTCAAACTCAGGGAA |
| | | ACCTAAATCTAGTTATAGACAAGGCAATCCTGAGCC |
| | | AAGCCGAAGTAGTAATTAGTAAGTTAACAATAGATG |
| | | ACTTACAACTAATCGGAAGGTGCAGAGACTCGACGG |
| | | GAGCTACCCTAACGTCAAGACGAGGGTAAAGAGAG |
| | | AGTCCAATTCTCAAAGCCAATAcacaaacacaa |
| 277 | T25 L10a | gctgcgagccACGGACTTAAATAATTGAGCCTTAAAGAA |
| | | GAAATTCTTTAAGTGGATGCTCTCAAACTCAGGGAA |
| | | ACCTAAATCTAGTTATAGACAAGGCAATCCTGAGCC |
| | | AAGCCGAAGTAGTAATTAGTAAGTTAACAATAGATG |
| | | ACTTACAACTAATCGGAAGGTGCAGAGACTCGACGG |
| | | GAGCTACCCTAACGTCAAGACGAGGGTAAAGAGAG |
| | | AGTCCAATTCTCAAAGCCAATAcacaaacacaacttatatact |
| 278 | T25 L20a | gctgcgagccACGGACTTAAATAATTGAGCCTTAAAGAA |
| | | GAAATTCTTTAAGTGGATGCTCTCAAACTCAGGGAA |
| | | ACCTAAATCTAGTTATAGACAAGGCAATCCTGAGCC |
| | | AAGCCGAAGTAGTAATTAGTAAGTTAACAATAGATG |
| | | ACTTACAACTAATCGGAAGGTGCAGAGACTCGACGG |
| | | GAGCTACCCTAACGTCAAGACGAGGGTAAAGAGAG |
| | | AGTCCAATTCTCAAAGCCAATAcacaaacacaagagtataagtat |
| | | aatttcag |
| 279 | T25 L30a | gctgcgagccACGGACTTAAATAATTGAGCCTTAAAGAA |
| | (I80-10) | GAAATTCTTTAAGTGGATGCTCTCAAACTCAGGGAA |
| | [Control] | ACCTAAATCTAGTTATAGACAAGGCAATCCTGAGCC |
| | | AAGCCGAAGTAGTAATTAGTAAGTTAACAATAGATG |
| | | ACTTACAACTAATCGGAAGGTGCAGAGACTCGACGG |
| | | GAGCTACCCTAACGTCAAGACGAGGGTAAAGAGAG |
| | | AGTCCAATTCTCAAAGCCAATAcacaaacacaagtcatatactga |
| | | gtataagtataatttcag |
| 280 | T50 L10a | gctgcgagccACGGACTTAAATAATTGAGCCTTAAAGAA |
| | | GAAATTCTTTAAGTGGATGCTCTCAAACTCAGGGAA |
| | | ACCTAAATCTAGTTATAGACAAGGCAATCCTGAGCC |
| | | AAGCCGAAGTAGTAATTAGTAAGTTAACAATAGATG |
| | | ACTTACAACTAATCGGAAGGTGCAGAGACTCGACGG |
| | | GAGCTACCCTAACGTCAAGACGAGGGTAAAGAGAG |
| | | AGTCCAATTCTCAAAGCCAATAaacaaaaacaagctgacgcta |
| 281 | T50 L20a | gctgcgagccACGGACTTAAATAATTGAGCCTTAAAGAA |
| | | GAAATTCTTTAAGTGGATGCTCTCAAACTCAGGGAA |
| | | ACCTAAATCTAGTTATAGACAAGGCAATCCTGAGCC |
| | | AAGCCGAAGTAGTAATTAGTAAGTTAACAATAGATG |
| | | ACTTACAACTAATCGGAAGGTGCAGAGACTCGACGG |
| | | GAGCTACCCTAACGTCAAGACGAGGGTAAAGAGAG |
| | | AGTCCAATTCTCAAAGCCAATAaacaaaaacaagctgacgctagt |
| | | atgagtat |
| 282 | T50 L30a | gctgcgagccACGGACTTAAATAATTGAGCCTTAAAGAA |
| | | GAAATTCTTTAAGTGGATGCTCTCAAACTCAGGGAA |
| | | ACCTAAATCTAGTTATAGACAAGGCAATCCTGAGCC |
| | | AAGCCGAAGTAGTAATTAGTAAGTTAACAATAGATG |
| | | ACTTACAACTAATCGGAAGGTGCAGAGACTCGACGG |
| | | GAGCTACCCTAACGTCAAGACGAGGGTAAAGAGAG |
| | | AGTCCAATTCTCAAAGCCAATAaacaaaaacaagctgacgctagt |
| | | atgagtatagcttcacac |
| 283 | T75 L10a | gctgcgagccACGGACTTAAATAATTGAGCCTTAAAGAA |
| | | GAAATTCTTTAAGTGGATGCTCTCAAACTCAGGGAA |
| | | ACCTAAATCTAGTTATAGACAAGGCAATCCTGAGCC |
| | | AAGCCGAAGTAGTAATTAGTAAGTTAACAATAGATG |
| | | ACTTACAACTAATCGGAAGGTGCAGAGACTCGACGG |
| | | GAGCTACCCTAACGTCAAGACGAGGGTAAAGAGAG |
| | | AGTCCAATTCTCAAAGCCAATAaacaaaaacaagctcgcaccg |
| 284 | T75 L20a | gctgcgagccACGGACTTAAATAATTGAGCCTTAAAGAA |
| | | GAAATTCTTTAAGTGGATGCTCTCAAACTCAGGGAA |
| | | ACCTAAATCTAGTTATAGACAAGGCAATCCTGAGCC |
| | | AAGCCGAAGTAGTAATTAGTAAGTTAACAATAGATG |
| | | ACTTACAACTAATCGGAAGGTGCAGAGACTCGACGG |
| | | GAGCTACCCTAACGTCAAGACGAGGGTAAAGAGAG |
| | | AGTCCAATTCTCAAAGCCAATAaacaaaaacaagctcgcactgg |
| | | gtcggagcg |
| 285 | T75 L30a | gctgcgagccACGGACTTAAATAATTGAGCCTTAAAGAA |
| | | GAAATTCTTTAAGTGGATGCTCTCAAACTCAGGGAA |
| | | ACCTAAATCTAGTTATAGACAAGGCAATCCTGAGCC |
| | | AAGCCGAAGTAGTAATTAGTAAGTTAACAATAGATG |
| | | ACTTACAACTAATCGGAAGGTGCAGAGACTCGACGG |
| | | GAGCTACCCTAACGTCAAGACGAGGGTAAAGAGAG |
| | | AGTCCAATTCTCAAAGCCAATAaacaaaaacaagctcgcactgg |
| | | ccggaagctcgtcggagcg |
| 286 | T0 L0b | gctgcgagccACGGACTTAAATAATTGAGCCTTAAAGAA |
| | | GAAATTCTTTAAGTGGATGCTCTCAAACTCAGGGAA |
| | | ACCTAAATCTAGTTATAGACAAGGCAATCCTGAGCC |
| | | AAGCCGAAGTAGTAATTAGTAAGTTAACAATAGATG |
| | | ACTTACAACTAATCGGAAGGTGCAGAGACTCGACGG |
| | | GAGCTACCCTAACGTCcacaaacacaa |
| 287 | T25 L10b | gctgcgagccACGGACTTAAATAATTGAGCCTTAAAGAA |
| | | GAAATTCTTTAAGTGGATGCTCTCAAACTCAGGGAA |
| | | ACCTAAATCTAGTTATAGACAAGGCAATCCTGAGCC |
| | | AAGCCGAAGTAGTAATTAGTAAGTTAACAATAGATG |
| | | ACTTACAACTAATCGGAAGGTGCAGAGACTCGACGG |
| | | GAGCTACCCTAACGTCcacaaacacaacttatatact |
| 288 | T25 L20b | gctgcgagccACGGACTTAAATAATTGAGCCTTAAAGAA |
| | | GAAATTCTTTAAGTGGATGCTCTCAAACTCAGGGAA |
| | | ACCTAAATCTAGTTATAGACAAGGCAATCCTGAGCC |
| | | AAGCCGAAGTAGTAATTAGTAAGTTAACAATAGATG |
| | | ACTTACAACTAATCGGAAGGTGCAGAGACTCGACGG |
| | | GAGCTACCCTAACGTCcacaaacacaagagtataagtataatttcag |
| 289 | T25 L30b | gctgcgagccACGGACTTAAATAATTGAGCCTTAAAGAA |
| | (I80-10) | GAAATTCTTTAAGTGGATGCTCTCAAACTCAGGGAA |
| | [Control] | ACCTAAATCTAGTTATAGACAAGGCAATCCTGAGCC |
| | | AAGCCGAAGTAGTAATTAGTAAGTTAACAATAGATG |
| | | ACTTACAACTAATCGGAAGGTGCAGAGACTCGACGG |
| | | GAGCTACCCTAACGTCcacaaacacaagtcatatactgagtataagtata |
| | | atttcag |
| 290 | T50 L10b | gctgcgagccACGGACTTAAATAATTGAGCCTTAAAGAA |
| | | GAAATTCTTTAAGTGGATGCTCTCAAACTCAGGGAA |
| | | ACCTAAATCTAGTTATAGACAAGGCAATCCTGAGCC |
| | | AAGCCGAAGTAGTAATTAGTAAGTTAACAATAGATG |
| | | ACTTACAACTAATCGGAAGGTGCAGAGACTCGACGG |
| | | GAGCTACCCTAACGTCaacaaaaacaagctgacgcta |
| 291 | T50 L20b | gctgcgagccACGGACTTAAATAATTGAGCCTTAAAGAA |
| | | GAAATTCTTTAAGTGGATGCTCTCAAACTCAGGGAA |
| | | ACCTAAATCTAGTTATAGACAAGGCAATCCTGAGCC |
| | | AAGCCGAAGTAGTAATTAGTAAGTTAACAATAGATG |
| | | ACTTACAACTAATCGGAAGGTGCAGAGACTCGACGG |
| | | GAGCTACCCTAACGTCaacaaaaacaagctgacgctagtatgagtat |
| 292 | T50 L30b | gctgcgagccACGGACTTAAATAATTGAGCCTTAAAGAA |
| | | GAAATTCTTTAAGTGGATGCTCTCAAACTCAGGGAA |
| | | ACCTAAATCTAGTTATAGACAAGGCAATCCTGAGCC |
| | | AAGCCGAAGTAGTAATTAGTAAGTTAACAATAGATG |
| | | ACTTACAACTAATCGGAAGGTGCAGAGACTCGACGG |
| | | GAGCTACCCTAACGTCaacaaaaacaagctgacgctagtatgagtatag |
| | | cttcacac |
| 293 | T75 L10b | gctgcgagccACGGACTTAAATAATTGAGCCTTAAAGAA |
| | | GAAATTCTTTAAGTGGATGCTCTCAAACTCAGGGAA |
| | | ACCTAAATCTAGTTATAGACAAGGCAATCCTGAGCC |
| | | AAGCCGAAGTAGTAATTAGTAAGTTAACAATAGATG |
| | | ACTTACAACTAATCGGAAGGTGCAGAGACTCGACGG |
| | | GAGCTACCCTAACGTCaacaaaaacaagctcgcaccg |
| 294 | T75 L20b | gctgcgagccACGGACTTAAATAATTGAGCCTTAAAGAA |
| | | GAAATTCTTTAAGTGGATGCTCTCAAACTCAGGGAA |
| | | ACCTAAATCTAGTTATAGACAAGGCAATCCTGAGCC |
| | | AAGCCGAAGTAGTAATTAGTAAGTTAACAATAGATG |
| | | ACTTACAACTAATCGGAAGGTGCAGAGACTCGACGG |
| | | GAGCTACCCTAACGTCaacaaaaacaagctcgcactgggtcggagcg |
| 295 | T75 L30b | gctgcgagccACGGACTTAAATAATTGAGCCTTAAAGAA |
| | | GAAATTCTTTAAGTGGATGCTCTCAAACTCAGGGAA |
| | | ACCTAAATCTAGTTATAGACAAGGCAATCCTGAGCC |
| | | AAGCCGAAGTAGTAATTAGTAAGTTAACAATAGATG |
| | | ACTTACAACTAATCGGAAGGTGCAGAGACTCGACGG |
| | | GAGCTACCCTAACGTCaacaaaaacaagctcgcactggccggaagct |
| | | cgtcggagcg |
| 296 | T25 L30 | ACGGACTTAAATAATTGAGCCTTAAAGAAGAAATTC |
| | I0-0 | TTTAAGTGGATGCTCTCAAACTCAGGGAAACCTAAA |
| | | TCTAGTTATAGACAAGGCAATCCTGAGCCAAGCCGA |
| | | AGTAGTAATTAGTAAGTTAACAAcacaaacacaagtcatatact |
| | | gagtataagtataatttcag |
| 297 | T25 L30a I0-0 | ACGGACTTAAATAATTGAGCCTTAAAGAAGAAATTC |
| | | TTTAAGTGGATGCTCTCAAACTCAGGGAAACCTAAA |
| | | TCTAGTTATAGACAAGGCAATCCTGAGCCAAGCCGA |
| | | AGTAGTAATTAGTAAGTTAACAATAGATGACTTACA |
| | | ACTAATCGGAAGGTGCAGAGACTCGACGGGAGCTA |
| | | CCCTAACGTCAAGACGAGGGTAAAGAGAGAGTCCA |
| | | ATTCTCAAAGCCAATAcacaaacacaagtcatatactgagtataagtata |
| | | atttcag |
| 298 | T25 L30a I25- | ctgtttaatcACGGACTTAAATAATTGAGCCTTAAAGAAG |
| | 10 | AAATTCTTTAAGTGGATGCTCTCAAACTCAGGGAAA |
| | | CCTAAATCTAGTTATAGACAAGGCAATCCTGAGCCA |
| | | AGCCGAAGTAGTAATTAGTAAGTTAACAATAGATGA |
| | | CTTACAACTAATCGGAAGGTGCAGAGACTCGACGGG |
| | | AGCTACCCTAACGTCAAGACGAGGGTAAAGAGAGA |
| | | GTCCAATTCTCAAAGCCAATAcacaaacacaagtcatatactgagt |
| | | ataagtataatttcag |
| 299 | T25 L30a I25- | cgtaatttatattgtgaatcACGGACTTAAATAATTGAGCCTTAA |
| | 20 | AGAAGAAATTCTTTAAGTGGATGCTCTCAAACTCAG |
| | | GGAAACCTAAATCTAGTTATAGACAAGGCAATCCTG |
| | | AGCCAAGCCGAAGTAGTAATTAGTAAGTTAACAATA |
| | | GATGACTTACAACTAATCGGAAGGTGCAGAGACTCG |
| | | ACGGGAGCTACCCTAACGTCAAGACGAGGGTAAAG |
| | | AGAGAGTCCAATTCTCAAAGCCAATAcacaaacacaagtcat |
| | | atactgagtataagtataatttcag |
| 300 | T25 L30a I50- | gctatgatccACGGACTTAAATAATTGAGCCTTAAAGAAG |
| | 10 | AAATTCTTTAAGTGGATGCTCTCAAACTCAGGGAAA |
| | | CCTAAATCTAGTTATAGACAAGGCAATCCTGAGCCA |
| | | AGCCGAAGTAGTAATTAGTAAGTTAACAATAGATGA |
| | | CTTACAACTAATCGGAAGGTGCAGAGACTCGACGGG |
| | | AGCTACCCTAACGTCAAGACGAGGGTAAAGAGAGA |
| | | GTCCAATTCTCAAAGCCAATAcacaaacacaagtcatatactgagt |
| | | ataagtataatttcag |
| 301 | T25 L30a I50- | cggatattatgctgcgatccACGGACTTAAATAATTGAGCCTTA |
| | 20 | AAGAAGAAATTCTTTAAGTGGATGCTCTCAAACTCA |
| | | GGGAAACCTAAATCTAGTTATAGACAAGGCAATCCT |
| | | GAGCCAAGCCGAAGTAGTAATTAGTAAGTTAACAAT |
| | | AGATGACTTACAACTAATCGGAAGGTGCAGAGACTC |
| | | GACGGGAGCTACCCTAACGTCAAGACGAGGGTAAA |
| | | GAGAGAGTCCAATTCTCAAAGCCAATAcacaaacacaagtc |
| | | atatactgagtataagtataatttcag |
| 302 | T25 L30a I80- | cggtaggcgcgctgcgagccACGGACTTAAATAATTGAGCCTT |
| | 20 | AAAGAAGAAATTCTTTAAGTGGATGCTCTCAAACTC |
| | | AGGGAAACCTAAATCTAGTTATAGACAAGGCAATCC |
| | | TGAGCCAAGCCGAAGTAGTAATTAGTAAGTTAACAA |
| | | TAGATGACTTACAACTAATCGGAAGGTGCAGAGACT |
| | | CGACGGGAGCTACCCTAACGTCAAGACGAGGGTAA |
| | | AGAGAGAGTCCAATTCTCAAAGCCAATAcacaaacacaag |
| | | tcatatactgagtataagtataatttcag |
| 303 | T25 L30b I0- | ACGGACTTAAATAATTGAGCCTTAAAGAAGAAATTC |
| | 0 | TTTAAGTGGATGCTCTCAAACTCAGGGAAACCTAAA |
| | | TCTAGTTATAGACAAGGCAATCCTGAGCCAAGCCGA |
| | | AGTAGTAATTAGTAAGTTAACAATAGATGACTTACA |
| | | ACTAATCGGAAGGTGCAGAGACTCGACGGGAGCTA |
| | | CCCTAACGTCcacaaacacaagtcatatactgagtataagtataatttcag |
| 304 | T25 L30b | ctgtttaatcACGGACTTAAATAATTGAGCCTTAAAGAAG |
| | I25-10 | AAATTCTTTAAGTGGATGCTCTCAAACTCAGGGAAA |
| | | CCTAAATCTAGTTATAGACAAGGCAATCCTGAGCCA |
| | | AGCCGAAGTAGTAATTAGTAAGTTAACAATAGATGA |
| | | CTTACAACTAATCGGAAGGTGCAGAGACTCGACGGG |
| | | AGCTACCCTAACGTCcacaaacacaagtcatatactgagtataagtataat |
| | | ttcag |
| 305 | T25 L30b | cgtaatttatattgtgaatcACGGACTTAAATAATTGAGCCTTAA |
| | I25-20 | AGAAGAAATTCTTTAAGTGGATGCTCTCAAACTCAG |
| | | GGAAACCTAAATCTAGTTATAGACAAGGCAATCCTG |
| | | AGCCAAGCCGAAGTAGTAATTAGTAAGTTAACAATA |
| | | GATGACTTACAACTAATCGGAAGGTGCAGAGACTCG |
| | | ACGGGAGCTACCCTAACGTCcacaaacacaagtcatatactgagtat |
| | | aagtataatttcag |
| 306 | T25 L30b | gctatgatccACGGACTTAAATAATTGAGCCTTAAAGAAG |
| | I50-10 | AAATTCTTTAAGTGGATGCTCTCAAACTCAGGGAAA |
| | | CCTAAATCTAGTTATAGACAAGGCAATCCTGAGCCA |
| | | AGCCGAAGTAGTAATTAGTAAGTTAACAATAGATGA |
| | | CTTACAACTAATCGGAAGGTGCAGAGACTCGACGGG |
| | | AGCTACCCTAACGTCcacaaacacaagtcatatactgagtataagtataat |
| | | ttcag |
| 307 | T25 L30b | cggatattatgctgcgatccACGGACTTAAATAATTGAGCCTTA |
| | I50-20 | AAGAAGAAATTCTTTAAGTGGATGCTCTCAAACTCA |
| | | GGGAAACCTAAATCTAGTTATAGACAAGGCAATCCT |
| | | GAGCCAAGCCGAAGTAGTAATTAGTAAGTTAACAAT |
| | | AGATGACTTACAACTAATCGGAAGGTGCAGAGACTC |
| | | GACGGGAGCTACCCTAACGTCcacaaacacaagtcatatactgagt |
| | | ataagtataatttcag |
| 308 | T25 L30b | cggtaggcgcgctgcgagccACGGACTTAAATAATTGAGCCTT |
| | I80-20 | AAAGAAGAAATTCTTTAAGTGGATGCTCTCAAACTC |
| | | AGGGAAACCTAAATCTAGTTATAGACAAGGCAATCC |
| | | TGAGCCAAGCCGAAGTAGTAATTAGTAAGTTAACAA |
| | | TAGATGACTTACAACTAATCGGAAGGTGCAGAGACT |
| | | CGACGGGAGCTACCCTAACGTCcacaaacacaagtcatatactga |
| | | gtataagtataatttcag |

In some embodiments, a spacer and 3′ intron fragment is a spacer and intron fragments having sequences as listed in Table 7.

TABLE 8
Cleavage site sequences.

| SEQ ID | | |
|---|---|---|
| NO: | Cleavage site | Sequence |
| 309 | 2A-like sequence | YHADYYKQRLIHDVEMNPGP |
| 310 | 2A-like sequence | HYAGYFADLLIHDIETNPGP |
| 311 | 2A-like sequence | QCTNYALLKLAGDVESNPGP |
| 312 | 2A-like sequence | ATNFSLLKQAGDVEENPGP |
| 313 | 2A-like sequence | AARQMLLLLSGDVETNPGP |
| 314 | 2A-like sequence | RAEGRGSLLTCGDVEENPGP |
| 315 | 2A-like sequence | TRAEIEDELIRAGIESNPGP |
| 316 | 2A-like sequence | AKFQIDKILISGDVELNPGP |
| 317 | 2A-like sequence | SSIIRTKMLVSGDVEENPGP |
| 318 | 2A-like sequence | CDAQRQKLLLSGDIEQNPGP |
| 319 | 2A-like sequence | YPIDFGGFLVKADSEFNPGP |
| 320 | P2A | GSGATNFSLLKQAGDVEENPGP |
| 321 | F2A | GSGEGRGSLLTCGDVEENPGP |
| 322 | E2A | GSGQCTNYALLKLAGDVESNPGP |
| 323 | T2A | GSGVKQTLNFDLLKLAGDVESNP |
| | | GP |
| 324 | 2A conserved sequence | GDVEXNPGP |

TABLE 9
SARS-CoV-2 protein sequences.

| SEQ ID | SARS-CoV-2 | |
|---|---|---|
| NO: | proteins | Sequence |
| 325 | spike | MFVFLVLLPLVSSQCVNLTTRTQLPPAYTNSFTRGVY |
| | glycoprotein | YPDKVFRSSVLHSTQDLFLPFFSNVTWFHAIHVSGING |
| | | TKRFDNPVLPFNDGVYFASTEKSNIIRGWIFGTTLDSK |
| | | TQSLLIVNNATNVVIKVCEFQFCNDPFLGVYYHKNNK |
| | | SWMESEFRVYSSANNCTFEYVSQPFLMDLEGKQGNF |
| | | KNLREFVFKNIDGYFKIYSKHTPINLVRDLPQGFSALEP |
| | | LVDLPIGINITRFQTLLALHRSYLTPGDSSSGWTAGAA |
| | | AYYVGYLQPRTFLLKYNENGTITDAVDCALDPLSETK |
| | | CTLKSFTVEKGIYQTSNFRVQPTESIVRFPNITNLCPFG |
| | | EVFNATRFASVYAWNRKRISNCVADYSVLYNSASFST |
| | | FKCYGVSPTKLNDLCFTNVYADSFVIRGDEVRQIAPG |
| | | QTGKIADYNYKLPDDFTGCVIAWNSNNLDSKVGGNY |
| | | NYLYRLFRKSNLKPFERDISTEIYQAGSTPCNGVEGEN |
| | | CYFPLQSYGFQPTNGVGYQPYRVVVLSFELLHAPATV |
| | | CGPKKSTNLVKNKCVNFNFNGLTGTGVLTESNKKFLP |
| | | FQQFGRDIADTTDAVRDPQTLEILDITPCSFGGVSVITP |
| | | GTNTSNQVAVLYQDVNCTEVPVAIHADQLTPTWRVY |
| | | STGSNVFQTRAGCLIGAEHVNNSYECDIPIGAGICASY |
| | | QTQTNSPRRARSVASQSIIAYTMSLGAENSVAYSNNSI |
| | | AIPTNFTISVTTEILPVSMTKTSVDCTMYICGDSTECSN |
| | | LLLQYGSFCTQLNRALTGIAVEQDKNTQEVFAQVKQI |
| | | YKTPPIKDFGGFNFSQILPDPSKPSKRSFIEDLLFNKVTL |
| | | ADAGFIKQYGDCLGDIAARDLICAQKENGLTVLPPLLT |
| | | DEMIAQYTSALLAGTITSGWTFGAGAALQIPFAMQMA |
| | | YRENGIGVTQNVLYENQKLIANQFNSAIGKIQDSLSST |
| | | ASALGKLQDVVNQNAQALNTLVKQLSSNFGAISSVLN |
| | | DILSRLDKVEAEVQIDRLITGRLQSLQTYVTQQLIRAA |
| | | EIRASANLAATKMSECVLGQSKRVDFCGKGYHLMSFP |
| | | QSAPHGVVFLHVTYVPAQEKNFTTAPAICHDGKAHFP |
| | | REGVFVSNGTHWFVTQRNFYEPQIITTDNTFVSGNCD |
| | | VVIGIVNNTVYDPLQPELDSFKEELDKYFKNHTSPDVD |
| | | LGDISGINASVVNIQKEIDRLNEVAKNLNESLIDLQELG |
| | | KYEQYIKWPWYIWLGFIAGLIAIVMVTIMLCCMTSCC |
| | | SCLKGCCSCGSCCKFDEDDSEPVLKGVKLHYT |
| 326 | ORF1ab | MESLVPGFNEKTHVQLSLPVLQVRDVLVRGFGDSVEE |
| | polyprotein | VLSEARQHLKDGTCGLVEVEKGVLPQLEQPYVFIKRS |
| | | DARTAPHGHVMVELVAELEGIQYGRSGETLGVLVPH |
| | | VGEIPVAYRKVLLRKNGNKGAGGHSYGADLKSFDLG |
| | | DELGTDPYEDFQENWNTKHSSGVTRELMRELNGGAY |
| | | TRYVDNNFCGPDGYPLECIKDLLARAGKASCTLSEQL |
| | | DFIDTKRGVYCCREHEHEIAWYTERSEKSYELQTPFEI |
| | | KLAKKFDTFNGECPNFVFPLNSIIKTIQPRVEKKKLDGF |
| | | MGRIRSVYPVASPNECNQMCLSTLMKCDHCGETSWQ |
| | | TGDFVKATCEFCGTENLTKEGATTCGYLPQNAVVKIY |
| | | CPACHNSEVGPEHSLAEYHNESGLKTILRKGGRTIAFG |
| | | GCVFSYVGCHNKCAYWVPRASANIGCNHTGVVGEGS |
| | | EGLNDNLLEILQKEKVNINIVGDFKLNEEIAIILASFSAS |
| | | TSAFVETVKGLDYKAFKQIVESCGNFKVTKGKAKKG |
| | | AWNIGEQKSILSPLYAFASEAARVVRSIFSRTLETAQNS |
| | | VRVLQKAAITILDGISQYSLRLIDAMMFTSDLATNNLV |
| | | VMAYITGGVVQLTSQWLTNIFGTVYEKLKPVLDWLE |
| | | EKFKEGVEFLRDGWEIVKFISTCACEIVGGQIVTCAKEI |
| | | KESVQTFFKLVNKFLALCADSIIIGGAKLKALNLGETF |
| | | VTHSKGLYRKCVKSREETGLLMPLKAPKEIIFLEGETL |
| | | PTEVLTEEVVLKTGDLQPLEQPTSEAVEAPLVGTPVCI |
| | | NGLMLLEIKDTEKYCALAPNMMVTNNTFTLKGGAPT |
| | | KVTFGDDTVIEVQGYKSVNITFELDERIDKVLNEKCSA |
| | | YTVELGTEVNEFACVVADAVIKTLQPVSELLTPLGIDL |
| | | DEWSMATYYLFDESGEFKLASHMYCSFYPPDEDEEEG |
| | | DCEEEEFEPSTQYEYGTEDDYQGKPLEFGATSAALQPE |
| | | EEQEEDWLDDDSQQTVGQQDGSEDNQTTTIQTIVEVQ |
| | | PQLEMELTPVVQTIEVNSFSGYLKLTDNVYIKNADIVE |
| | | EAKKVKPTVVVNAANVYLKHGGGVAGALNKATNNA |
| | | MQVESDDYIATNGPLKVGGSCVLSGHNLAKHCLHVV |
| | | GPNVNKGEDIQLLKSAYENFNQHEVLLAPLLSAGIFGA |
| | | DPIHSLRVCVDTVRTNVYLAVFDKNLYDKLVSSFLEM |
| | | KSEKQVEQKIAEIPKEEVKPFITESKPSVEQRKQDDKKI |
| | | KACVEEVTTTLEETKFLTENLLLYIDINGNLHPDSATL |
| | | VSDIDITFLKKDAPYIVGDVVQEGVLTAVVIPTKKAGG |
| | | TTEMLAKALRKVPTDNYITTYPGQGLNGYTVEEAKTV |
| | | LKKCKSAFYILPSIISNEKQEILGTVSWNLREMLAHAEE |
| | | TRKLMPVCVETKAIVSTIQRKYKGIKIQEGVVDYGARF |
| | | YFYTSKTTVASLINTLNDLNETLVTMPLGYVTHGLNL |
| | | EEAARYMRSLKVPATVSVSSPDAVTAYNGYLTSSSKT |
| | | PEEHFIETISLAGSYKDWSYSGQSTQLGIEFLKRGDKSV |
| | | YYTSNPTTFHLDGEVITFDNLKTLLSLREVRTIKVFTTV |
| | | DNINLHTQVVDMSMTYGQQFGPTYLDGADVTKIKPH |
| | | NSHEGKTFYVLPNDDTLRVEAFEYYHTTDPSFLGRYM |
| | | SALNHTKKWKYPQVNGLTSIKWADNNCYLATALLTL |
| | | QQIELKFNPPALQDAYYRARAGEAANFCALILAYCNK |
| | | TVGELGDVRETMSYLFQHANLDSCKRVLNVVCKTCG |
| | | QQQTTLKGVEAVMYMGTLSYEQFKKGVQIPCTCGKQ |
| | | ATKYLVQQESPFVMMSAPPAQYELKHGTFTCASEYTG |
| | | NYQCGHYKHITSKETLYCIDGALLTKSSEYKGPITDVF |
| | | YKENSYTTTIKPVTYKLDGVVCTEIDPKLDNYYKKDN |
| | | SYFTEQPIDLVPNQPYPNASFDNFKFVCDNIKFADDLN |
| | | QLTGYKKPASRELKVTFFPDLNGDVVAIDYKHYTPSF |
| | | KKGAKLLHKPIVWHVNNATNKATYKPNTWCIRCLWS |
| | | TKPVETSNSFDVLKSEDAQGMDNLACEDLKPVSEEVV |
| | | ENPTIQKDVLECNVKTTEVVGDIILKPANNSLKITEEV |
| | | GHTDLMAAYVDNSSLTIKKPNELSRVLGLKTLATHGL |
| | | AAVNSVPWDTIANYAKPFLNKVVSTTTNIVTRCLNRV |
| | | CTNYMPYFFTLLLQLCTFTRSTNSRIKASMPTTIAKNT |
| | | VKSVGKFCLEASFNYLKSPNFSKLINIIIWFLLLSVCLG |
| | | SLIYSTAALGVLMSNLGMPSYCTGYREGYLNSTNVTI |
| | | ATYCTGSIPCSVCLSGLDSLDTYPSLETIQITISSFKWDL |
| | | TAFGLVAEWFLAYILFTRFFYVLGLAAIMQLFFSYFAV |
| | | HFISNSWLMWLIINLVQMAPISAMVRMYIFFASFYYV |
| | | WKSYVHVVDGCNSSTCMMCYKRNRATRVECTTIVN |
| | | GVRRSFYVYANGGKGFCKLHNWNCVNCDTFCAGSTF |
| | | ISDEVARDLSLQFKRPINPTDQSSYIVDSVTVKNGSIHL |
| | | YFDKAGQKTYERHSLSHFVNLDNLRANNTKGSLPINV |
| | | IVFDGKSKCEESSAKSASVYYSQLMCQPILLLDQALVS |
| | | DVGDSAEVAVKMFDAYVNTFSSTFNVPMEKLKTLVA |
| | | TAEAELAKNVSLDNVLSTFISAARQGFVDSDVETKDV |
| | | VECLKLSHQSDIEVTGDSCNNYMLTYNKVENMTPRD |
| | | LGACIDCSARHINAQVAKSHNIALIWNVKDFMSLSEQ |
| | | LRKQIRSAAKKNNLPFKLTCATTRQVVNVVTTKIALK |
| | | GGKIVNNWLKQLIKVTLVFLFVAAIFYLITPVHVMSKH |
| | | TDFSSEIIGYKAIDGGVTRDIASTDTCFANKHADFDTW |
| | | FSQRGGSYTNDKACPLIAAVITREVGFVVPGLPGTILR |
| | | TTNGDFLHFLPRVFSAVGNICYTPSKLIEYTDFATSAC |
| | | VLAAECTIFKDASGKPVPYCYDTNVLEGSVAYESLRP |
| | | DTRYVLMDGSIIQFPNTYLEGSVRVVTTFDSEYCRHGT |
| | | CERSEAGVCVSTSGRWVLNNDYYRSLPGVFCGVDAV |
| | | NLLTNMFTPLIQPIGALDISASIVAGGIVAIVVTCLAYY |
| | | FMRFRRAFGEYSHVVAFNTLLFLMSFTVLCLTPVYSFL |
| | | PGVYSVIYLYLTFYLTNDVSFLAHIQWMVMFTPLVPF |
| | | WITIAYIICISTKHFYWFFSNYLKRRVVFNGVSFSTFEE |
| | | AALCTFLINKEMYLKLRSDVLLPLTQYNRYLALYNK |
| | | YKYFSGAMDTTSYREAACCHLAKALNDFSNSGSDVL |
| | | YQPPQTSITSAVLQSGFRKMAFPSGKVEGCMVQVTCG |
| | | TTTLNGLWLDDVVYCPRHVICTSEDMLNPNYEDLLIR |
| | | KSNHNFLVQAGNVQLRVIGHSMQNCVLKLKVDTANP |
| | | KTPKYKFVRIQPGQTFSVLACYNGSPSGVYQCAMRPN |
| | | FTIKGSFLNGSCGSVGFNIDYDCVSFCYMHHMELPTG |
| | | VHAGTDLEGNFYGPFVDRQTAQAAGTDTTITVNVLA |
| | | WLYAAVINGDRWFLNRFTTTLNDFNLVAMKYNYEPL |
| | | TQDHVDILGPLSAQTGIAVLDMCASLKELLQNGMNGR |
| | | TILGSALLEDEFTPFDVVRQCSGVTFQSAVKRTIKGTH |
| | | HWLLLTILTSLLVLVQSTQWSLFFFLYENAFLPFAMGII |
| | | AMSAFAMMFVKHKHAFLCLFLLPSLATVAYFNMVY |
| | | MPASWVMRIMTWLDMVDTSLSGFKLKDCVMYASAV |
| | | VLLILMTARTVYDDGARRVWTLMNVLTLVYKVYYG |
| | | NALDQAISMWALIISVTSNYSGVVTTVMFLARGIVFM |
| | | CVEYCPIFFITGNTLQCIMLVYCFLGYFCTCYFGLFCLL |
| | | NRYFRLTLGVYDYLVSTQEFRYMNSQGLLPPKNSIDA |
| | | FKLNIKLLGVGGKPCIKVATVQSKMSDVKCTSVVLLS |
| | | VLQQLRVESSSKLWAQCVQLHNDILLAKDTTEAFEK |
| | | MVSLLSVLLSMQGAVDINKLCEEMLDNRATLQAIASE |
| | | FSSLPSYAAFATAQEAYEQAVANGDSEVVLKKLKKSL |
| | | NVAKSEFDRDAAMQRKLEKMADQAMTQMYKQARS |
| | | EDKRAKVTSAMQTMLFTMLRKLDNDALNNIINNARD |
| | | GCVPLNIIPLTTAAKLMVVIPDYNTYKNTCDGTTFTYA |
| | | SALWEIQQVVDADSKIVQLSEISMDNSPNLAWPLIVTA |
| | | LRANSAVKLQNNELSPVALRQMSCAAGTTQTACTDD |
| | | NALAYYNTTKGGRFVLALLSDLQDLKWARFPKSDGT |
| | | GTIYTELEPPCRFVTDTPKGPKVKYLYFIKGLNNLNRG |
| | | MVLGSLAATVRLQAGNATEVPANSTVLSFCAFAVDA |
| | | AKAYKDYLASGGQPITNCVKMLCTHTGTGQAITVTPE |
| | | ANMDQESFGGASCCLYCRCHIDHPNPKGFCDLKGKY |
| | | VQIPTTCANDPVGFTLKNTVCTVCGMWKGYGCSCDQ |
| | | LREPMLQSADAQSFLNRVCGVSAARLTPCGTGTSTDV |
| | | VYRAFDIYNDKVAGFAKFLKTNCCRFQEKDEDDNLID |
| | | SYFVVKRHTFSNYQHEETIYNLLKDCPAVAKHDFFKF |
| | | RIDGDMVPHISRQRLTKYTMADLVYALRHFDEGNCD |
| | | TLKEILVTYNCCDDDYFNKKDWYDFVENPDILRVYA |
| | | NLGERVRQALLKTVQFCDAMRNAGIVGVLTLDNQDL |
| | | NGNWYDFGDFIQTTPGSGVPVVDSYYSLLMPILTLTR |
| | | ALTAESHVDTDLTKPYIKWDLLKYDFTEERLKLFDRY |
| | | FKYWDQTYHPNCVNCLDDRCILHCANFNVLFSTVFPP |
| | | TSFGPLVRKIFVDGVPFVVSTGYHFRELGVVHNQDVN |
| | | LHSSRLSFKELLVYAADPAMHAASGNLLLDKRTTCFS |
| | | VAALINNVAFQTVKPGNFNKDFYDFAVSKGFFKEGSS |
| | | VELKHFFFAQDGNAAISDYDYYRYNLPTMCDIRQLLF |
| | | VVEVVDKYFDCYDGGCINANQVIVNNLDKSAGFPFN |
| | | KWGKARLYYDSMSYEDQDALFAYTKRNVIPTITQMN |
| | | LKYAISAKNRARTVAGVSICSTMTNRQFHQKLLKSIA |
| | | ATRGATVVIGTSKFYGGWHNMLKTVYSDVENPHLMG |
| | | WDYPKCDRAMPNMLRIMASLVLARKHTTCCSLSHRF |
| | | YRLANECAQVLSEMVMCGGSLYVKPGGTSSGDATTA |
| | | YANSVFNICQAVTANVNALLSTDGNKIADKYVRNLQ |
| | | HRLYECLYRNRDVDTDFVNEFYAYLRKHFSMMILSD |
| | | DAVVCFNSTYASQGLVASIKNFKSVLYYQNNVFMSEA |
| | | KCWTETDLTKGPHEFCSQHTMLVKQGDDYVYLPYPD |
| | | PSRILGAGCFVDDIVKTDGTLMIERFVSLAIDAYPLTK |
| | | HPNQEYADVFHLYLQYIRKLHDELTGHMLDMYSVML |
| | | TNDNTSRYWEPEFYEAMYTPHTVLQAVGACVLCNSQ |
| | | TSLRCGACIRRPFLCCKCCYDHVISTSHKLVLSVNPYV |
| | | CNAPGCDVTDVTQLYLGGMSYYCKSHKPPISFPLCAN |
| | | GQVFGLYKNTCVGSDNVTDFNAIATCDWTNAGDYIL |
| | | ANTCTERLKLFAAETLKATEETFKLSYGIATVREVLSD |
| | | RELHLSWEVGKPRPPLNRNYVFTGYRVTKNSKVQIGE |
| | | YTFEKGDYGDAVVYRGTTTYKLNVGDYFVLTSHTVM |
| | | PLSAPTLVPQEHYVRITGLYPTLNISDEFSSNVANYQK |
| | | VGMQKYSTLQGPPGTGKSHFAIGLALYYPSARIVYTA |
| | | CSHAAVDALCEKALKYLPIDKCSRIIPARARVECFDKF |
| | | KVNSTLEQYVFCTVNALPETTADIVVFDEISMATNYD |
| | | LSVVNARLRAKHYVYIGDPAQLPAPRTLLTKGTLEPE |
| | | YFNSVCRLMKTIGPDMFLGTCRRCPAEIVDTVSALVY |
| | | DNKLKAHKDKSAQCFKMFYKGVITHDVSSAINRPQIG |
| | | VVREFLTRNPAWRKAVFISPYNSQNAVASKILGLPTQT |
| | | VDSSQGSEYDYVIFTQTTETAHSCNVNRFNVAITRAK |
| | | VGILCIMSDRDLYDKLQFTSLEIPRRNVATLQAENVTG |
| | | LFKDCSKVITGLHPTQAPTHLSVDTKFKTEGLCVDIPGI |
| | | PKDMTYRRLISMMGFKMNYQVNGYPNMFITREEAIR |
| | | HVRAWIGFDVEGCHATREAVGTNLPLQLGFSTGVNL |
| | | VAVPTGYVDTPNNTDFSRVSAKPPPGDQFKHLIPLMY |
| | | KGLPWNVVRIKIVQMLSDTLKNLSDRVVFVLWAHGF |
| | | ELTSMKYFVKIGPERTCCLCDRRATCESTASDTYACW |
| | | HHSIGFDYVYNPFMIDVQQWGFTGNLQSNHDLYCQV |
| | | HGNAHVASCDAIMTRCLAVHECFVKRVDWTIEYPIIG |
| | | DELKINAACRKVQHMVVKAALLADKFPVLHDIGNPK |
| | | AIKCVPQADVEWKFYDAQPCSDKAYKIEELFYSYATH |
| | | SDKFTDGVCLFWNCNVDRYPANSIVCRFDTRVLSNLN |
| | | LPGCDGGSLYVNKHAFHTPAFDKSAFVNLKQLPFFYY |
| | | SDSPCESHGKQVVSDIDYVPLKSATCITRQNLGGAVCR |
| | | HHANEYRLYLDAYNMMISAGFSLWVYKQFDTYNLW |
| | | NTFTRLQSLENVAFNVVNKGHFDGQQGEVPVSIINNT |
| | | VYTKVDGVDVELFENKTTLPVNVAFELWAKRNIKPVP |
| | | EVKILNNLGVDIAANTVIWDYKRDAPAHISTIGVCSMT |
| | | DIAKKPTETICAPLTVFFDGRVDGQVDLFRNARNGVLI |
| | | TEGSVKGLQPSVGPKQASLNGVTLIGEAVKTQFNYYK |
| | | KVDGVVQQLPETYFTQSRNLQEFKPRSQMEIDFLELA |
| | | MDEFIERYKLEGYAFEHIVYGDFSHSQLGGLHLLIGLA |
| | | KRFKESPFELEDFIPMDSTVKNYFITDAQTGSSKCVCS |
| | | VIDLLLDDFVEIIKSQDLSVVSKVVKVTIDYTEISFMLW |
| | | CKDGHVETFYPKLQSSQAWQPGVAMPNLYKMQRML |
| | | LEKCDLQNYGDSATLPKGIMMNVAKYTQLCQYLNTL |
| | | TLAVPYNMRVIHFGAGSDKGVAPGTAVLRQWLPTGT |
| | | LLVDSDLNDFVSDADSTLIGDCATVHTANKWDLIISD |
| | | MYDPKTKNVTKENDSKEGFFTYICGFIQQKLALGGSV |
| | | AIKITEHSWNADLYKLMGHFAWWTAFVTNVNASSSE |
| | | AFLIGCNYLGKPREQIDGYVMHANYIFWRNTNPIQLSS |
| | | YSLFDMSKFPLKLRGTAVMSLKEGQINDMILSLLSKG |
| | | RLIIRENNRVVISSDVLVNN |
| 327 | ORF1a | MESLVPGFNEKTHVQLSLPVLQVRDVLVRGFGDSVEE |
| | polyprotein | VLSEARQHLKDGTCGLVEVEKGVLPQLEQPYVFIKRS |
| | | DARTAPHGHVMVELVAELEGIQYGRSGETLGVLVPH |
| | | VGEIPVAYRKVLLRKNGNKGAGGHSYGADLKSFDLG |
| | | DELGTDPYEDFQENWNTKHSSGVTRELMRELNGGAY |
| | | TRYVDNNFCGPDGYPLECIKDLLARAGKASCTLSEQL |
| | | DFIDTKRGVYCCREHEHEIAWYTERSEKSYELQTPFEI |
| | | KLAKKFDTENGECPNFVFPLNSIIKTIQPRVEKKKLDGF |
| | | MGRIRSVYPVASPNECNQMCLSTLMKCDHCGETSWQ |
| | | TGDFVKATCEFCGTENLTKEGATTCGYLPQNAVVKY |
| | | CPACHNSEVGPEHSLAEYHNESGLKTILRKGGRTIAFG |
| | | GCVFSYVGCHNKCAYWVPRASANIGCNHTGVVGEGS |
| | | EGLNDNLLEILQKEKVNINIVGDFKLNEEIAIILASFSAS |
| | | TSAFVETVKGLDYKAFKQIVESCGNFKVTKGKAKKG |
| | | AWNIGEQKSILSPLYAFASEAARVVRSIFSRTLETAQNS |
| | | VRVLQKAAITILDGISQYSLRLIDAMMFTSDLATNNLV |
| | | VMAYITGGVVQLTSQWLTNIFGTVYEKLKPVLDWLE |
| | | EKFKEGVEFLRDGWEIVKFISTCACEIVGGQIVTCAKEI |
| | | KESVQTFFKLVNKFLALCADSIIIGGAKLKALNLGETF |
| | | VTHSKGLYRKCVKSREETGLLMPLKAPKEIIFLEGETL |
| | | PTEVLTEEVVLKTGDLQPLEQPTSEAVEAPLVGTPVCI |
| | | NGLMLLEIKDTEKYCALAPNMMVTNNTFTLKGGAPT |
| | | KVTFGDDTVIEVQGYKSVNITFELDERIDKVLNEKCSA |
| | | YTVELGTEVNEFACVVADAVIKTLQPVSELLTPLGIDL |
| | | DEWSMATYYLFDESGEFKLASHMYCSFYPPDEDEEEG |
| | | DCEEEEFEPSTQYEYGTEDDYQGKPLEFGATSAALQPE |
| | | EEQEEDWLDDDSQQTVGQQDGSEDNQTTTIQTIVEVQ |
| | | PQLEMELTPVVQTIEVNSFSGYLKLTDNVYIKNADIVE |
| | | EAKKVKPTVVVNAANVYLKHGGGVAGALNKATNNA |
| | | MQVESDDYIATNGPLKVGGSCVLSGHNLAKHCLHVV |
| | | GPNVNKGEDIQLLKSAYENFNQHEVLLAPLLSAGIFGA |
| | | DPIHSLRVCVDTVRTNVYLAVEDKNLYDKLVSSFLEM |
| | | KSEKQVEQKIAEIPKEEVKPFITESKPSVEQRKQDDKKI |
| | | KACVEEVTTTLEETKFLTENLLLYIDINGNLHPDSATL |
| | | VSDIDITFLKKDAPYIVGDVVQEGVLTAVVIPTKKAGG |
| | | TTEMLAKALRKVPTDNYITTYPGQGLNGYTVEEAKTV |
| | | LKKCKSAFYILPSIISNEKQEILGTVSWNLREMLAHABE |
| | | TRKLMPVCVETKAIVSTIQRKYKGIKIQEGVVDYGARF |
| | | YFYTSKTTVASLINTLNDLNETLVTMPLGYVTHGLNL |
| | | EEAARYMRSLKVPATVSVSSPDAVTAYNGYLTSSSKT |
| | | PEEHFIETISLAGSYKDWSYSGQSTQLGIEFLKRGDKSV |
| | | YYTSNPTTFHLDGEVITFDNLKTLLSLREVRTIKVFTTV |
| | | DNINLHTQVVDMSMTYGQQFGPTYLDGADVTKIKPH |
| | | NSHEGKTFYVLPNDDTLRVEAFEYYHTTDPSFLGRYM |
| | | SALNHTKKWKYPQVNGLTSIKWADNNCYLATALLTL |
| | | QQIELKFNPPALQDAYYRARAGEAANFCALILAYCNK |
| | | TVGELGDVRETMSYLFQHANLDSCKRVLNVVCKTCG |
| | | QQQTTLKGVEAVMYMGTLSYEQFKKGVQIPCTCGKQ |
| | | ATKYLVQQESPFVMMSAPPAQYELKHGTFTCASEYTG |
| | | NYQCGHYKHITSKETLYCIDGALLTKSSEYKGPITDVF |
| | | YKENSYTTTIKPVTYKLDGVVCTEIDPKLDNYYKKDN |
| | | SYFTEQPIDLVPNQPYPNASFDNFKFVCDNIKFADDLN |
| | | QLTGYKKPASRELKVTFFPDLNGDVVAIDYKHYTPSF |
| | | KKGAKLLHKPIVWHVNNATNKATYKPNTWCIRCLWS |
| | | TKPVETSNSFDVLKSEDAQGMDNLACEDLKPVSEEVV |
| | | ENPTIQKDVLECNVKTTEVVGDIILKPANNSLKITEEV |
| | | GHTDLMAAYVDNSSLTIKKPNELSRVLGLKTLATHGL |
| | | AAVNSVPWDTIANYAKPFLNKVVSTTTNIVTRCLNRV |
| | | CTNYMPYFFTLLLQLCTFTRSTNSRIKASMPTTIAKNT |
| | | VKSVGKFCLEASFNYLKSPNFSKLINIIIWFLLLSVCLG |
| | | SLIYSTAALGVLMSNLGMPSYCTGYREGYLNSTNVTI |
| | | ATYCTGSIPCSVCLSGLDSLDTYPSLETIQITISSFKWDL |
| | | TAFGLVAEWFLAYILFTRFFYVLGLAAIMQLFFSYFAV |
| | | HFISNSWLMWLIINLVQMAPISAMVRMYIFFASFYYV |
| | | WKSYVHVVDGCNSSTCMMCYKRNRATRVECTTIVN |
| | | GVRRSFYVYANGGKGFCKLHNWNCVNCDTFCAGSTF |
| | | ISDEVARDLSLQFKRPINPTDQSSYIVDSVTVKNGSIHL |
| | | YFDKAGQKTYERHSLSHFVNLDNLRANNTKGSLPINV |
| | | IVFDGKSKCEESSAKSASVYYSQLMCQPILLLDQALVS |
| | | DVGDSAEVAVKMFDAYVNTFSSTFNVPMEKLKTLVA |
| | | TAEAELAKNVSLDNVLSTFISAARQGFVDSDVETKDV |
| | | VECLKLSHQSDIEVTGDSCNNYMLTYNKVENMTPRD |
| | | LGACIDCSARHINAQVAKSHNIALIWNVKDFMSLSEQ |
| | | LRKQIRSAAKKNNLPFKLTCATTRQVVNVVTTKIALK |
| | | GGKIVNNWLKQLIKVTLVFLFVAAIFYLITPVHVMSKH |
| | | TDFSSEIIGYKAIDGGVTRDIASTDTCFANKHADFDTW |
| | | FSQRGGSYTNDKACPLIAAVITREVGFVVPGLPGTILR |
| | | TTNGDFLHFLPRVFSAVGNICYTPSKLIEYTDFATSAC |
| | | VLAAECTIFKDASGKPVPYCYDTNVLEGSVAYESLRP |
| | | DTRYVLMDGSIIQFPNTYLEGSVRVVTTFDSEYCRHGT |
| | | CERSEAGVCVSTSGRWVLNNDYYRSLPGVFCGVDAV |
| | | NLLTNMFTPLIQPIGALDISASIVAGGIVAIVVTCLAYY |
| | | FMRFRRAFGEYSHVVAFNTLLFLMSFTVLCLTPVYSFL |
| | | PGVYSVIYLYLTFYLTNDVSFLAHIQWMVMFTPLVPF |
| | | WITIAYIICISTKHFYWFFSNYLKRRVVFNGVSFSTFEE |
| | | AALCTFLINKEMYLKLRSDVLLPLTQYNRYLALYNK |
| | | YKYFSGAMDTTSYREAACCHLAKALNDFSNSGSDVL |
| | | YQPPQTSITSAVLQSGFRKMAFPSGKVEGCMVQVTCG |
| | | TTTLNGLWLDDVVYCPRHVICTSEDMLNPNYEDLLIR |
| | | KSNHNFLVQAGNVQLRVIGHSMQNCVLKLKVDTANP |
| | | KTPKYKFVRIQPGQTFSVLACYNGSPSGVYQCAMRPN |
| | | FTIKGSFLNGSCGSVGFNIDYDCVSFCYMHHMELPTG |
| | | VHAGTDLEGNFYGPFVDRQTAQAAGTDTTITVNVLA |
| | | WLYAAVINGDRWFLNRFTTTLNDFNLVAMKYNYEPL |
| | | TQDHVDILGPLSAQTGIAVLDMCASLKELLQNGMNGR |
| | | TILGSALLEDEFTPFDVVRQCSGVTFQSAVKRTIKGTH |
| | | HWLLLTILTSLLVLVQSTQWSLFFFLYENAFLPFAMGII |
| | | AMSAFAMMFVKHKHAFLCLFLLPSLATVAYFNMVY |
| | | MPASWVMRIMTWLDMVDTSLSGFKLKDCVMYASAV |
| | | VLLILMTARTVYDDGARRVWTLMNVLTLVYKVYYG |
| | | NALDQAISMWALIISVTSNYSGVVTTVMFLARGIVEM |
| | | CVEYCPIFFITGNTLQCIMLVYCFLGYFCTCYFGLFCLL |
| | | NRYFRLTLGVYDYLVSTQEFRYMNSQGLLPPKNSIDA |
| | | FKLNIKLLGVGGKPCIKVATVQSKMSDVKCTSVVLLS |
| | | VLQQLRVESSSKLWAQCVQLHNDILLAKDTTEAFEK |
| | | MVSLLSVLLSMQGAVDINKLCEEMLDNRATLQAIASE |
| | | FSSLPSYAAFATAQEAYEQAVANGDSEVVLKKLKKSL |
| | | NVAKSEFDRDAAMQRKLEKMADQAMTQMYKQARS |
| | | EDKRAKVTSAMQTMLFTMLRKLDNDALNNIINNARD |
| | | GCVPLNIIPLTTAAKLMVVIPDYNTYKNTCDGTTFTYA |
| | | SALWEIQQVVDADSKIVQLSEISMDNSPNLAWPLIVTA |
| | | LRANSAVKLQNNELSPVALRQMSCAAGTTQTACTDD |
| | | NALAYYNTTKGGRFVLALLSDLQDLKWARFPKSDGT |
| | | GTIYTELEPPCRFVTDTPKGPKVKYLYFIKGLNNLNRG |
| | | MVLGSLAATVRLQAGNATEVPANSTVLSFCAFAVDA |
| | | AKAYKDYLASGGQPITNCVKMLCTHTGTGQAITVTPE |
| | | ANMDQESFGGASCCLYCRCHIDHPNPKGFCDLKGKY |
| | | VQIPTTCANDPVGFTLKNTVCTVCGMWKGYGCSCDQ |
| | | LREPMLQSADAQSFLNGFAV |
| 328 | ORF3a protein | MDLFMRIFTIGTVTLKQGEIKDATPSDFVRATATIPIQA |
| | | SLPFGWLIVGVALLAVFQSASKIITLKKRWQLALSKGV |
| | | HFVCNLLLLFVTVYSHLLLVAAGLEAPFLYLYALVYF |
| | | LQSINFVRIIMRLWLCWKCRSKNPLLYDANYFLCWHT |
| | | NCYDYCIPYNSVTSSIVITSGDGTTSPISEHDYQIGGYT |
| | | EKWESGVKDCVVLHSYFTSDYYQLYSTQLSTDTGVE |
| | | HVTFFIYNKIVDEPEEHVQIHTIDGSSGVVNPVMEPIYD |
| | | EPTTTTSVPL |
| 329 | envelope | MYSFVSEETGTLIVNSVLLFLAFVVFLLVTLAILTALRL |
| | protein | CAYCCNIVNVSLVKPSFYVYSRVKNLNSSRVPDLLV |
| 330 | membrane | MADSNGTITVEELKKLLEQWNLVIGFLFLTWICLLQFA |
| | glycoprotein | YANRNRFLYIIKLIFLWLLWPVTLACFVLAAVYRINWI |
| | | TGGIAIAMACLVGLMWLSYFIASFRLFARTRSMWSFN |
| | | PETNILLNVPLHGTILTRPLLESELVIGAVILRGHLRIAG |
| | | HHLGRCDIKDLPKEITVATSRTLSYYKLGASQRVAGD |
| | | SGFAAYSRYRIGNYKLNTDHSSSSDNIALLVQ |
| 331 | ORF6 protein | MFHLVDFQVTIAEILLIIMRTFKVSIWNLDYIINLIIKNL |
| | | SKSLTENKYSQLDEEQPMEID |
| 332 | ORF7a protein | MKIILFLALITLATCELYHYQECVRGTTVLLKEPCSSGT |
| | | YEGNSPFHPLADNKFALTCFSTQFAFACPDGVKHVYQ |
| | | LRARSVSPKLFIRQEEVQELYSPIFLIVAAIVFITLCFTL |
| | | KRKTE |
| 333 | ORF7b protein | MIELSLIDFYLCFLAFLLFLVLIMLIIFWFSLELQDHNET |
| | | CHA |
| 334 | ORF8 protein | MKFLVFLGIITTVAAFHQECSLQSCTQHQPYVVDDPCP |
| | | IHFYSKWYIRVGARKSAPLIELCVDEAGSKSPIQYIDIG |
| | | NYTVSCLPFTINCQEPKLGSLVVRCSFYEDFLEYHDVR |
| | | VVLDFI |
| 335 | nucleocapsid | MSDNGPQNQRNAPRITFGGPSDSTGSNQNGERSGARS |
| | phosphoprotein | KQRRPQGLPNNTASWFTALTQHGKEDLKFPRGQGVPI |
| | | NTNSSPDDQIGYYRRATRRIRGGDGKMKDLSPRWYFY |
| | | YLGTGPEAGLPYGANKDGIIWVATEGALNTPKDHIGT |
| | | RNPANNAAIVLQLPQGTTLPKGFYAEGSRGGSQASSR |
| | | SSSRSRNSSRNSTPGSSRGTSPARMAGNGGDAALALLL |
| | | LDRLNQLESKMSGKGQQQQGQTVTKKSAAEASKKPR |
| | | QKRTATKAYNVTQAFGRRGPEQTQGNFGDQELIRQG |
| | | TDYKHWPQIAQFAPSASAFFGMSRIGMEVTPSGTWLT |
| | | YTGAIKLDDKDPNFKDQVILLNKHIDAYKTFPPTEPKK |
| | | DKKKKADETQALPQRQKKQQTVTLLPAADLDDFSKQ |
| | | LQQSMSSADSTQA |
| 336 | ORF10 protein | MGYINVFAFPFTIYSLLLCRMNSRNYIAQVDVVNFNLT |

In some embodiments, an antigenic polypeptide is a SARS-CoV-2 protein, a fragment of a SARS-CoV-2 protein, or is derived from a SARS-CoV-2 protein or a fragment thereof. In some embodiments, the antigenic polypeptide may consist of, but is not limited to, SARS-CoV2 spike protein, Nsp1-Nsp16, ORF3a, ORF6, ORF7a, ORFb, ORF8, ORF 10, SARS-CoV2 envelope protein, SARS-CoV2 Membrane protein, SARS-CoV2 nucleocapsid protein or an immunogenic fragment of SARS-CoV2 spike protein.

In some embodiments, an antigen contains all or part of a sequence on Table 9. In some embodiments, a peptide contains a sequence with at least about 70%, 75%, 80%, 85%, 90%, 91%, 92%, 93%, 94%, 95%, 96%, 97%, 98%, 99%, or 99.5% similarity to a sequence on Table 9. In some embodiments, a circular RNA vaccine contains RNA encoding more than one antigen. In some embodiments, a circular RNA vaccine contains RNA encoding at least about 2, 3, 4, 5, 6, 7, 8, 9, 10, 11, 12, 13, 14, or 15 antigens. In some embodiments, a circular RNA polynucleotide encodes more than one antigen. In some embodiments, a circular RNA RNA polynucleotide encodes at least about 2, 3, 4, 5, 6, 7, 8, 9, 10, 11, 12, 13, 14, or 15 antigens.

TABLE 10
Adjuvant polypeptides

| SEQ ID | | |
|---|---|---|
| NO: | Adjuvant Protein | Sequence |
| 337 | BCSP31 | MKFGSKIRRLAVAAVAGAIALGASFAVAQAPTFFRI |
| | (BCSP_BRUME) | GTGGTAGTYYPIGGLIANAISGAGEKGVPGLVATA |
| | | VSSNGSVANINAIKSGALESGFTQSDVAYWAYNGT |
| | | GLYDGKGKVEDLRLLATLYPETIHIVARKDANIKSV |
| | | ADLKGKRVSLDEPGSGTIVDARIVLEAYGLTEDDIK |
| | | AEHLKPGPAGERLKDGALDAYFFVGGYPTGAISEL |
| | | AISNGISLVPISGPEADKILEKYSFFSKDVVPAGAYK |
| | | DVAETPTLAVAAQWVTSAKQPDDLIYNITKVLWNE |
| | | DTRKALDAGHAKGKLIKLDSATSSLGIPLHPGAERF |
| | | YKEAGVLK |
| 338 | MOMP | MKKLLKSALLFAATGSALSLQALPVGNPAEPSLLID |
| | (MOMP6_CHLP6) | GTMWEGASGDPCDPCATWCDAISIRAGYYGDYVF |
| | | DRVLKVDVNKTFSGMAATPTQATGNASNTNQPEA |
| | | NGRPNIAYGRHMQDAEWFSNAAFLALNIWDREDIF |
| | | CTLGASNGYFKASSAAFNLVGLIGFSAASSISTDLP |
| | | MQLPNVGITQGVVEFYTDTSFSWSVGARGALWEC |
| | | GCATLGAEFQYAQSNPKIEMLNVTSSPAQFVIHKPR |
| | | GYKGASSNFPLPITAGTTEATDTKSATIKYHEWQVG |
| | | LALSYRLNMLVPYIGVNWSRATFDADTIRIAQPKLK |
| | | SEILNITTWNPSLIGSTTALPNNSGKDVLSDVLQIASI |
| | | QINKMKSRKACGVAVGATLIDADKWSITGEARLIN |
| | | ERAAHMNAQFRF |
| 339 | Flagellin | MAQVINTNSLSLITQNNINKNQSALSSSIERLSSGLRI |
| | (FLIC_ECOLI | NSAKDDAAGQAIANRFTSNIKGLTQAARNANDGIS |
| | (strain K12)) | VAQTTEGALSEINNNLQRVRELTVQATTGTNSESDL |
| | | SSIQDEIKSRLDEIDRVSGQTQFNGVNVLAKNGSMK |
| | | IQVGANDNQTITIDLKQIDAKTLGLDGFSVKNNDTV |
| | | TTSAPVTAFGATTTNNIKLTGITLSTEAATDTGGTNP |
| | | ASIEGVYTDNGNDYYAKITGGDNDGKYYAVTVAN |
| | | DGTVTMATGATANATVTDANTTKATTITSGGTPVQ |
| | | IDNTAGSATANLGAVSLVKLQDSKGNDTDTYALK |
| | | DTNGNLYAADVNETTGAVSVKTITYTDSSGAASSP |
| | | TAVKLGGDDGKTEVVDIDGKTYDSADLNGGNLQT |
| | | GLTAGGEALTAVANGKTTDPLKALDDAIASVDKFR |
| | | SSLGAVQNRLDSAVINLNNTTTNLSEAQSRIQDAD |
| | | YATEVSNMSKAQIIQQAGNSVLAKANQVPQQVLSL |
| | | LQG |
| 340 | IFN-alpha | MASPFALLMVLVVLSCKSSCSLGCDLPETHSLDNR |
| | (IFNA1_HUMAN | RTLMLLAQMSRISPSSCLMDRHDFGFPQEEFDGNQF |
| | Interferon | QKAPAISVLHELIQQIFNLFTTKDSSAAWDEDLLDK |
| | alpha-1/13) | FCTELYQQLNDLEACVMQEERVGETPLMNADSILA |
| | | VKKYFRRITLYLTEKKYSPCAWEVVRAEIMRSLSLS |
| | | TNLQERLRRKE |
| 341 | IFN-gamma | MKYTSYILAFQLCIVLGSLGCYCQDPYVKEAENLK |
| | (IFNG_HUMAN | KYFNAGHSDVADNGTLFLGILKNWKEESDRKIMQS |
| | Interferon | QIVSFYFKLFKNFKDDQSIQKSVETIKEDMNVKFFN |
| | gamma) | SNKKKRDDFEKLTNYSVTDLNVQRKAIHELIQVMA |
| | | ELSPAAKTGKRKRSQMLFRGRRASQ |
| 342 | IL-2 | MYRMQLLSCIALSLALVTNSAPTSSSTKKTQLQLEH |
| | (IL2_HUMAN | LLLDLQMILNGINNYKNPKLTRMLTFKFYMPKKAT |
| | Interleukin-2) | ELKHLQCLEEELKPLEEVLNLAQSKNFHLRPRDLIS |
| | | NINVIVLELKGSETTFMCEYADETATIVEFLNRWITF |
| | | CQSIISTLT |
| 343 | IL-15 | MRISKPHLRSISIQCYLCLLLNSHFLTEAGIHVFILGC |
| | (IL15_HUMAN | FSAGLPKTEANWVNVISDLKKIEDLIQSMHIDATLY |
| | Interleukin-15) | TESDVHPSCKVTAMKCFLLELQVISLESGDASIHDT |
| | | VENLIILANNSLSSNGNVTESGCKECEELEEKNIKEF |
| | | LQSFVHIVQMFINTS |
| 344 | IL-18 | MAAEPVEDNCINFVAMKFIDNTLYFIAEDDENLESD |
| | (IL18_HUMAN | YFGKLESKLSVIRNLNDQVLFIDQGNRPLFEDMTDS |
| | Interleukin-18) | DCRDNAPRTIFIISMYKDSQPRGMAVTISVKCEKIST |
| | | LSCENKIISFKEMNPPDNIKDTKSDIIFFQRSVPGHDN |
| | | KMQFESSSYEGYFLACEKERDLFKLILKKEDELGDR |
| | | SIMFTVQNED |
| 345 | FLt3-ligand | MTVLAPAWSPTTYLLLLLLLSSGLSGTQDCSFQHSP |
| | | ISSDFAVKIRELSDYLLQDYPVTVASNLQDEELCGG |
| | | LWRLVLAQRWMERLKTVAGSKMQGLLERVNTEIH |
| | | FVTKCAFQPPPSCLRFVQTNISRLLQETSEQLVALKP |
| | | WITRQNFSRCLELQCQPDSSTLPPPWSPRPLEATAPT |
| | | APQPPLLLLLLLPVGLLLLAAAWCLHWQRTRRRTP |
| | | RPGEQVPPVPSPQDLLLVEH |
| 346 | anti-CTLA4 | QVQLVESGGGVVQPGRSLRLSCAASGFTFSSYTMH |
| | (ipilumimab) | WVRQAPGKGLEWVTFISYDGNNKYYADSVKGRFT |
| | | ISRDNSKNTLYLQMNSLRAEDTAIYYCARTGWLGP |
| | | FDYWGQGTLVTVSSASTKGPSVFPLAPSSKSTSGGT |
| | | AALGCLVKDYFPEPVTVSWNSGALTSGVHTFPAVL |
| | | QSSGLYSLSSVVTVPSSSLGTQTYICNVNHKPSNTK |
| | | VDKRVEPKSCDKTHTCPPCPAPELLGGPSVFLFPPK |
| | | PKDTLMISRTPEVTCVVVDVSHEDPEVKFNWYVDG |
| | | VEVHNAKTKPREEQYNSTYRVVSVLTVLHQDWLN |
| | | GKEYKCKVSNKALPAPIEKTISKAKGQPREPQVYTL |
| | | PPSRDELTKNQVSLTCLVKGFYPSDIAVEWESNGQP |
| | | ENNYKTTPPVLDSDGSFFLYSKLTVDKSRWQQGNV |
| | | FSCSVMHEALHNHYTQKSLSLSPGK |
| 347 | anti-PD1 | QVQLVESGGGVVQPGRSLRLDCKASGITFSNSGMH |
| | (nivolumab) | WVRQAPGKGLEWVAVIWYDGSKRYYADSVKGRF |
| | | TISRDNSKNTLFLQMNSLRAEDTAVYYCATNDDY |
| | | WGQGTLVTVSSASTKGPSVFPLAPCSRSTSESTAAL |
| | | GCLVKDYFPEPVTVSWNSGALTSGVHTFPAVLQSS |
| | | GLYSLSSVVTVPSSSLGTKTYTCNVDHKPSNTKVD |
| | | KRVESKYGPPCPPCPAPEFLGGPSVFLFPPKPKDTL |
| | | MISRTPEVTCVVVDVSQEDPEVQFNWYVDGVEVH |
| | | NAKTKPREEQFNSTYRVVSVLTVLHQDWLNGKEY |
| | | KCKVSNKGLPSSIEKTISKAKGQPREPQVYTLPPSQE |
| | | EMTKNQVSLTCLVKGFYPSDIAVEWESNGQPENNY |
| | | KTTPPVLDSDGSFFLYSRLTVDKSRWQEGNVFSCSV |
| | | MHEALHNHYTQKSLSLSLGK |
| 348 | anti-41BB | EVQLVQSGAEVKKPGESLRISCKGSGYSFSTYWISW |
| | (utomilumab) | VRQMPGKGLEWMGKIYPGDSYTNYSPSFQGQVTIS |
| | | ADKSISTAYLQWSSLKASDTAMYYCARGYGIFDY |
| | | WGQGTLVTVSSASTKGPSVFPLAPCSRSTSESTAAL |
| | | GCLVKDYFPEPVTVSWNSGALTSGVHTFPAVLQSS |
| | | GLYSLSSVVTVPSSNFGTQTYTCNVDHKPSNTKVD |
| | | KTVERKCCVECPPCPAPPVAGPSVFLFPPKPKDTLM |
| | | ISRTPEVTCVVVDVSHEDPEVQFNWYVDGVEVHN |
| | | AKTKPREEQFNSTFRVVSVLTVVHQDWLNGKEYK |
| | | CKVSNKGLPAPIEKTISKTKGQPREPQVYTLPPSREE |
| | | MTKNQVSLTCLVKGFYPSDIAVEWESNGQPENNYK |
| | | TTPPMLDSDGSFFLYSKLTVDKSRWQQGNVFSCSV |
| | | MHEALHNHYTQKSLSLSPGK |

In some embodiments, a polynucleotide or a protein encoded by a polynucleotide contains a sequence with at least about 70%, 75%, 80%, 85%, 90%, 91%, 92%, 93%, 94%, 95%, 96%, 97%, 98%, 99%, or 99.5% similarity to one or more sequences disclosed herein. In some embodiments, a polynucleotide or a protein encoded by a polynucleotide contains a sequence that is identical to one or more sequences disclosed herein. In some embodiments, an expression sequence encodes a protein that comprises or consists of a sequence with 70%, 75%, 80%, 85%, 90%, 91%, 92%, 93%, 94%, 95%, 96%, 97%, 98%, 99%, or 99.5% similarity to or is identical to a sequence in Table 8. In some embodiments, an expression sequence encodes a protein that comprises or consists of a sequence with 70%, 75%, 80%, 85%, 90%, 91%, 92%, 93%, 94%, 95%, 96%, 97%, 98%, 99%, or 99.5% similarity to or is identical to a sequence in Table 8, and an IRES that comprises or consists of a sequence with 70%, 75%, 80%, 85%, 90%, 91%, 92%, 93%, 94%, 95%, 96%, 97%, 98%, 99%, or 99.5% similarity to or is identical to a sequence in Table 1. In some embodiments, an expression sequence encodes a protein that comprises or consists of a sequence with 70%, 75%, 80%, 85%, 90%, 91%, 92%, 93%, 94%, 95%, 96%, 97%, 98%, 99%, or 99.5% similarity to or is identical to a sequence in Table 8, and 3′ and 5′ group I intron fragments that comprise or consist of corresponding sequences with 70%, 75%, 80%, 85%, 90%, 91%, 92%, 93%, 94%, 95%, 96%, 97%, 98%, 99%, or 99.5% similarity to or are identical to sequences in Tables 2 and 3, 4 and 5, or 6 and 7.

Preferred embodiments are described herein. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### EXAMPLES

Wesselhoeft et al., (2019) RNA Circularization Diminishes Immunogenicity and Can Extend Translation Duration In vivo. Molecular Cell. 74(3), 508-520 and Wesselhoeft et al., (2018) Engineering circular RNA for Potent and Stable Translation in Eukaryotic Cells. Nature Communications. 9, 2629 are incorporated by reference in their entirety.

The invention is further described in detail by reference to the following examples but are not intended to be limited to the following examples. These examples encompass any and all variations of the illustrations with the intention of providing those of ordinary skill in the art with complete disclosure and description of how to make and use the subject invention and are not intended to limit the scope of what is regarded as the invention.

### Example 1

### Example 1A: External Duplex Forming Regions Allow for Circularization of Long Precursor RNA Using the Permuted Intron Exon (PIE) Circularization Strategy

A 1.1 kb sequence containing a full-length encephalomyocarditis virus (EMCV) IRES, a Gaussia luciferase (GLuc) expression sequence, and two short exon fragments of the permuted intron-exon (PIE) construct were inserted between the 3′ and 5′ introns of the permuted group I catalytic intron in the thymidylate synthase (Td) gene of the T4 phage. Precursor RNA was synthesized by run-off transcription. Circularization was attempted by heating the precursor RNA in the presence of magnesium ions and GTP, but splicing products were not obtained.

Perfectly complementary 9 nucleotide and 19 nucleotide long duplex forming regions were designed and added at the 5′ and 3′ ends of the precursor RNA. Addition of these homology arms increased splicing efficiency from 0 to 16% for 9 nucleotide duplex forming regions and to 48% for 19 nucleotide duplex forming regions as assessed by disappearance of the precursor RNA band.

The splicing product was treated with RNase R. Sequencing across the putative splice junction of RNase R-treated splicing reactions revealed ligated exons, and digestion of the RNase R-treated splicing reaction with oligonucleotide-targeted RNase H produced a single band in contrast to two bands yielded by RNase H-digested linear precursor. This shows that circular RNA is a major product of the splicing reactions of precursor RNA containing the 9 or 19 nucleotide long external duplex forming regions.

### Example 1B: Spacers that Conserve Secondary Structures of IRES and PIE Splice Sites Increase Circularization Efficiency

A series of spacers was designed and inserted between the 3′ PIE splice site and the IRES. These spacers were designed to either conserve or disrupt secondary structures within intron sequences in the IRES, 3′ PIE splice site, and/or 5′ splice site. The addition of spacer sequences designed to conserve secondary structures resulted in 87% splicing efficiency, while the addition of a disruptive spacer sequences resulted in no detectable splicing.

### Example 2

### Example 2A: Internal Duplex Forming Regions in Addition to External Duplex Forming Regions Creates a Splicing Bubble and Allows for Translation of Several Expression Sequences

Spacers were designed to be unstructured, non-homologous to the intron and IRES sequences, and to contain spacer-spacer duplex forming regions. These were inserted between the 5′ exon and IRES and between the 3′ exon and expression sequence in constructs containing external duplex forming regions, EMCV IRES, and expression sequences for Gaussia luciferase (total length: 1289 nt), Firefly luciferase (2384 nt), eGFP (1451 nt), human erythropoietin (1313 nt), and Cas9 endonuclease (4934 nt). Circularization of all 5 constructs was achieved. Circularization of constructs utilizing T4 phage and Anabaena introns were roughly equal. Circularization efficiency was higher for shorter sequences. To measure translation, each construct was transfected into HEK293 cells. Gaussia and Firefly luciferase transfected cells produced a robust response as measured by luminescence, human erythropoietin was detectable in the media of cells transfected with erythropoietin circRNA, and EGFP fluorescence was observed from cells transfected with EGFP circRNA. Co-transfection of Cas9 circRNA with sgRNA directed against GFP into cells constitutively expressing GFP resulted in ablated fluorescence in up to 97% of cells in comparison to an sgRNA-only control.

### Example 2B: Use of CVB3 IRES Increases Protein Production

Constructs with internal and external duplex forming regions and differing IRES containing either Gaussia luciferase or Firefly luciferase expression sequences were made. Protein production was measured by luminescence in the supernatant of HEK293 cells 24 hours after transfection. The Coxsackievirus B3 (CVB3) IRES construct produced the most protein in both cases.

### Example 2C: Use of polyA or polyAC Spacers Increases Protein Production

Thirty nucleotide long polyA or polyAC spacers were added between the IRES and splice junction in a construct with each IRES that produced protein in example 2B. Gaussia luciferase activity was measured by luminescence in the supernatant of HEK293 cells 24 hours after transfection. Both spacers improved expression in every construct over control constructs without spacers.

### Example 3

HEK293 or HeLa Cells Transfected with Circular RNA Produce More Protein than Those Transfected with Comparable Unmodified or Modified Linear RNA.

HPLC-purified Gaussia luciferase-coding circRNA (CVB3-GLuc-pAC) was compared with a canonical unmodified 5′ methylguanosine-capped and 3′ polyA-tailed linear GLuc mRNA, and a commercially available nucleoside-modified (pseudouridine, 5-methylcytosine) linear GLuc mRNA (from Trilink). Luminescence was measured 24 h post-transfection, revealing that circRNA produced 811.2% more protein than the unmodified linear mRNA in HEK293 cells and 54.5% more protein than the modified mRNA. Similar results were obtained in HeLa cells and a comparison of optimized circRNA coding for human erythropoietin with linear mRNA modified with 5-methoxyuridine.

Luminescence data was collected over 6 days. In HEK293 cells, circRNA transfection resulted in a protein production half-life of 80 hours, in comparison with the 43 hours of unmodified linear mRNA and 45 hours of modified linear mRNA. In HeLa cells, circRNA transfection resulted in a protein production half-life of 116 hours, in comparison with the 44 hours of unmodified linear mRNA and 49 hours of modified linear mRNA. CircRNA produced substantially more protein than both the unmodified and modified linear mRNAs over its lifetime in both cell types.

### Example 4

### Example 4A: Purification of circRNA by RNase Digestion, HPLC Purification, and Phosphatase Treatment Decreases Immunogenicity. Completely Purified Circular RNA is Significantly Less Immunogenic than Unpurified or Partially Purified Circular RNA. Protein Expression Stability and Cell Viability are Dependent on Cell Type and Circular RNA Purity

Human embryonic kidney 293 (HEK293) and human lung carcinoma A549 cells were transfected with:
a. products of an unpurified GLuc circular RNA splicing reaction,
b. products of RNase R digestion of the splicing reaction,
c. products of RNase R digestion and HPLC purification of the splicing reaction, or
d. products of RNase digestion, HPLC purification, and phosphatase treatment of the splicing reaction.

RNase R digestion of splicing reactions was insufficient to prevent cytokine release in A549 cells in comparison to untransfected controls.

The addition of HPLC purification was also insufficient to prevent cytokine release, although there was a significant reduction in interleukin-6 (IL-6) and a significant increase in interferon-α1 (IFNα1) compared to the unpurified splicing reaction.

The addition of a phosphatase treatment after HPLC purification and before RNase R digestion dramatically reduced the expression of all upregulated cytokines assessed in A549 cells. Secreted monocyte chemoattractant protein 1 (MCP1), IL-6, IFNα1, tumor necrosis factor α (TNFα), and IFNγ inducible protein-10 (IP-10) fell to undetectable or un-transfected baseline levels.

There was no substantial cytokine release in HEK293 cells. A549 cells had increased GLuc expression stability and cell viability when transfected with higher purity circular RNA. Completely purified circular RNA had a stability phenotype similar to that of transfected 293 cells.

### Example 4B: Circular RNA does not Cause Significant Immunogenicity and is not a RIG-1 Ligand

A549 cells were transfected with:
a. unpurified circular RNA,
b. high molecular weight (linear and circular concatenations) RNA,
c. circular (nicked) RNA,
d. an early fraction of purified circular RNA (more overlap with nicked RNA peak),
e. a late fraction of purified circular RNA (less overlap with nicked RNA peak),
f. introns excised during circularization, or
g. vehicle (i.e. untransfected control).

Precursor RNA was separately synthesized and purified in the form of the splice site deletion mutant (DS) due to difficulties in obtaining suitably pure linear precursor RNA from the splicing reaction. Cytokine release and cell viability was measured in each case.

Robust IL-6, RANTES, and IP-10 release was observed in response to most of the species present within the splicing reaction, as well as precursor RNA. Early circRNA fractions elicited cytokine responses comparable to other non-circRNA fractions, indicating that even relatively small quantities of linear RNA contaminants are able to induce a substantial cellular immune response in A549 cells. Late circRNA fractions elicited no cytokine response in excess of that from untransfected controls. A549 cell viability 36 hours post-transfection was significantly greater for late circRNA fractions compared with all of the other fractions.

RIG-I and IFN-β1 transcript induction upon transfection of A549 cells with late circRNA HPLC fractions, precursor RNA or unpurified splicing reactions were analyzed. Induction of both RIG-I and IFN-β1 transcripts were weaker for late circRNA fractions than precursor RNA and unpurified splicing reactions. RNase R treatment of splicing reactions alone was not sufficient to ablate this effect. Addition of very small quantities of the RIG-I ligand 3p-hpRNA to circular RNA induced substantial RIG-I transcription. In HeLa cells, transfection of RNase R-digested splicing reactions induced RIG-I and IFN-β1, but purified circRNA did not. Overall, HeLa cells were less sensitive to contaminating RNA species than A549 cells.

A time course experiment monitoring RIG-I, IFN-β1, IL-6, and RANTES transcript induction within the first 8 hours after transfection of A549 cells with splicing reactions or fully purified circRNA did not reveal a transient response to circRNA. Purified circRNA similarly failed to induce pro-inflammatory transcripts in RAW264.7 murine macrophages.

A549 cells were transfected with purified circRNA containing an EMCV IRES and EGFP expression sequence. This failed to produce substantial induction of pro-inflammatory transcripts. These data demonstrate that non-circular components of the splicing reaction are responsible for the immunogenicity observed in previous studies and that circRNA is not a natural ligand for RIG-I.

### Example 5

### Circular RNA Avoids Detection by LRs.

TLR 3, 7, and 8 reporter cell lines were transfected with multiple linear or circular RNA constructs and secreted embryonic alkaline phosphatase (SEAP) was measured.

Linearized RNA was constructed by deleting the intron and homology arm sequences. The linear RNA constructs were then treated with phosphatase (in the case of capped RNAs, after capping) and purified by HPLC.

None of the attempted transfections produced a response in TLR7 reporter cells. TLR3 and TLR8 reporter cells were activated by capped linearized RNA, polyadenylated linearized RNA, the nicked circRNA HPLC fraction, and the early circRNA fraction. The late circRNA fraction and m1ψ-mRNA did not provoke TLR-mediated response in any cell line.

In a second experiment, circRNA was linearized using two methods: treatment of circRNA with heat in the presence of magnesium ions and DNA oligonucleotide-guided RNase H digestion. Both methods yielded a majority of full-length linear RNA with small amounts of intact circRNA. TLR3, 7, and 8 reporter cells were transfected with circular RNA, circular RNA degraded by heat, or circular RNA degraded by RNase H, and SEAP secretion was measured 36 hours after transfection. TLR8 reporter cells secreted SEAP in response to both forms of degraded circular RNA, but did not produce a greater response to circular RNA transfection than mock transfection. No activation was observed in TLR3 and TLR7 reporter cells for degraded or intact conditions, despite the activation of TLR3 by in vitro transcribed linearized RNA.

### Example 6

Unmodified Circular RNA Produces Increased Sustained In Vivo Protein Expression than Linear RNA.

Mice were injected and HEK293 cells were transfected with unmodified and m1ψ-modified human erythropoietin (hEpo) linear mRNAs and circRNAs. Equimolar transfection of m1ψ-mRNA and unmodified circRNA resulted in robust protein expression in HEK293 cells. hEpo linear mRNA and circRNA displayed similar relative protein expression patterns and cell viabilities in comparison to GLuc linear mRNA and circRNA upon equal weight transfection of HEK293 and A549 cells.

In mice, hEpo was detected in serum after the injection of hEpo circRNA or linear mRNA into visceral adipose. hEpo detected after the injection of unmodified circRNA decayed more slowly than that from unmodified or m1ψ-mRNA and was still present 42 hours post-injection. Serum hEpo rapidly declined upon the injection of unpurified circRNA splicing reactions or unmodified linear mRNA. Injection of unpurified splicing reactions produced a cytokine response detectable in serum that was not observed for the other RNAs, including purified circRNA.

### Example 7

### Circular RNA can be Effectively Delivered In Vivo or In Vitro Via Lipid Nanoparticles.

Purified circular RNA was formulated into lipid nanoparticles (LNPs) with the ionizable lipidoid cKK-E12 (Dong et al., 2014; Kauffman et al., 2015). The particles formed uniform multilamellar structures with an average size, polydispersity index, and encapsulation efficiency similar to that of particles containing commercially available control linear mRNA modified with 5moU.

Purified hEpo circRNA displayed greater expression than 5moU-mRNA when encapsulated in LNPs and added to HEK293 cells. Expression stability from LNP-RNA in HEK293 cells was similar to that of RNA delivered by transfection reagent, with the exception of a slight delay in decay for both 5moU-mRNA and circRNA. Both unmodified circRNA and 5moU-mRNA failed to activate RIG-I/IFN-β1 in vitro.

In mice, LNP-RNA was delivered by local injection into visceral adipose tissue or intravenous delivery to the liver. Serum hEpo expression from circRNA was lower but comparable with that from 5moU-mRNA 6 hours after delivery in both cases. Serum hEpo detected after adipose injection of unmodified LNP-circRNA decayed more slowly than that from LNP-5moU-mRNA, with a delay in expression decay present in serum that was similar to that noted in vitro, but serum hEpo after intravenous injection of LNP-circRNA or LNP-5moU-mRNA decayed at approximately the same rate. There was no increase in serum cytokines or local RIG-I, TNFα, or IL-6 transcript induction in any of these cases.

### Example 8

### Expression and Functional Stability by IRES in HEK293, HepG2, and 1C1C7 Cells

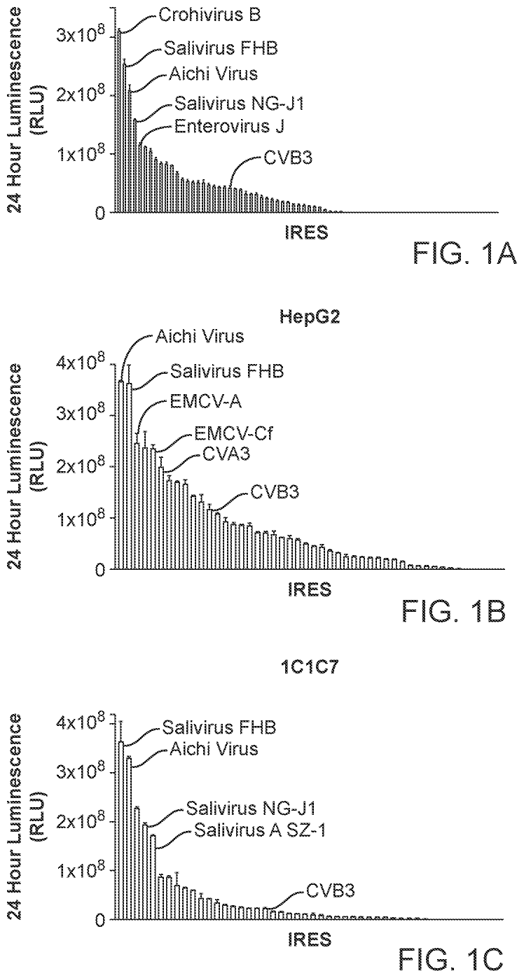

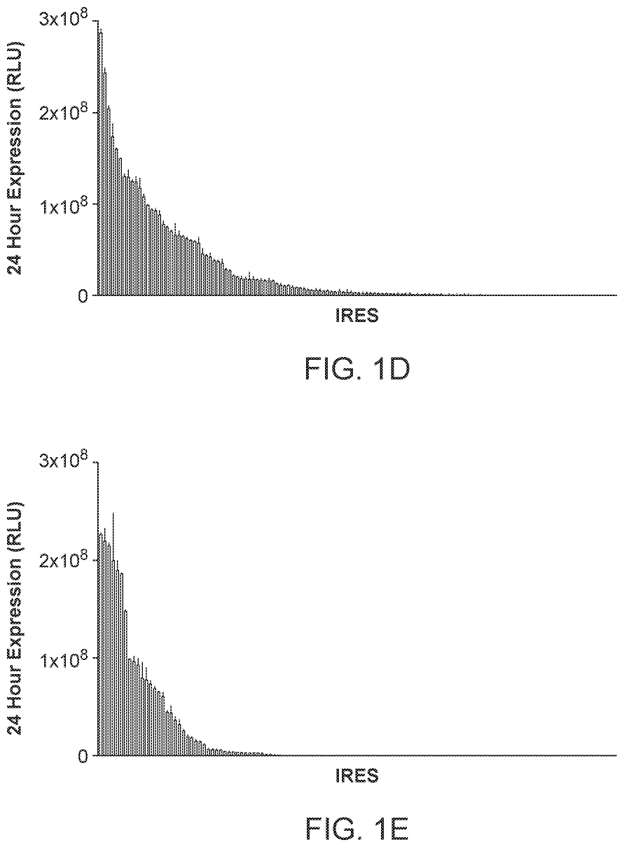

Constructs including anabaena intron/exon regions, a Gaussia luciferase expression sequence, and varying IRES were circularized. 100 ng of each circularization reaction was separately transfected into 20,000 HEK293 cells, HepG2 cells, and 1C1C7 cells using Lipofectamine MessengerMax. Luminescence in each supernatant was assessed after 24 hours as a measure of protein expression. In HEK293 cells, constructs including Crohivirus B, Salivirus FHB, Aichi Virus, Salivirus HG-J1, and Enterovirus J IRES produced the most luminescence at 24 hours (FIG. 1A). In HepG2 cells, constructs including Aichi Virus, Salivirus FHB, EMCV-Cf, and CVA3 IRES produced high luminescence at 24 hours (FIG. 1B). In 1C1C7 cells, constructs including Salivirus FHB, Aichi Virus, Salivirus NG-J1, and Salivirus A SZ-1 IRES produced high luminescence at 24 hours (FIG. 1C).

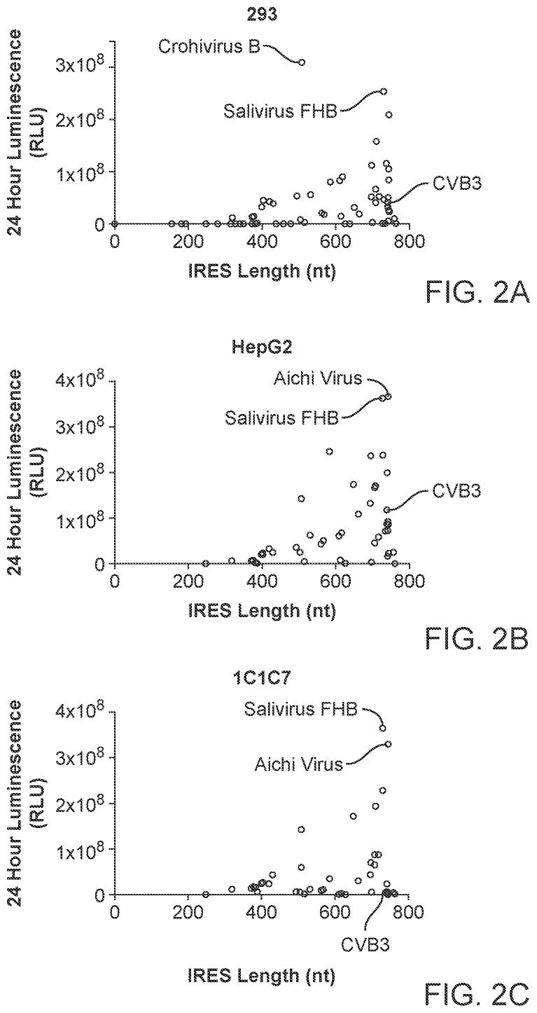

A trend of larger IRES producing greater luminescence at 24 hours was observed. Shorter total sequence length tends to increase circularization efficiency, so selecting a high expression and relatively short IRES may result in an improved construct. In HEK293 cells, a construct using the Crohivirus B IRES produced the highest luminescence, especially in comparison to other IRES of similar length (FIG. 2A). Expression from IRES constructs in HepG2 and 1C1C7 cells plotted against IRES size are in FIGS. 2B and 2C.

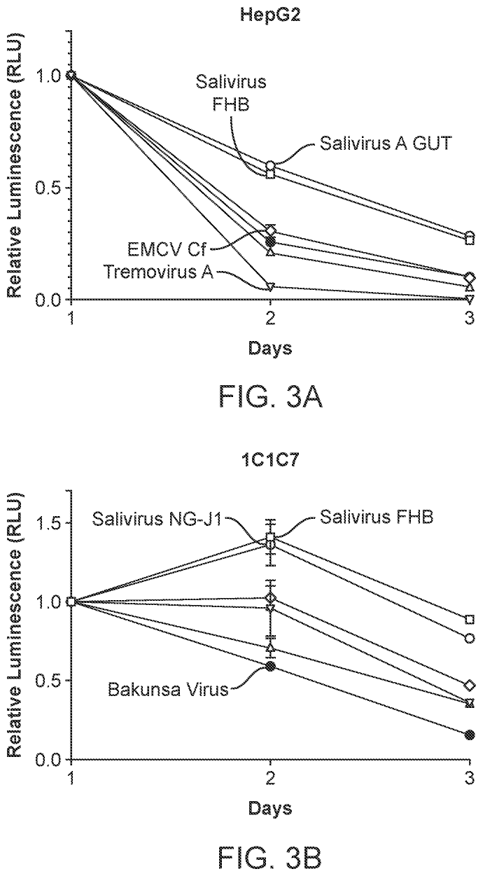

Functional stability of select IRES constructs in HepG2 and 1C1C7 cells were measured over 3 days. Luminescence from secreted Gaussia luciferase in supernatant was measured every 24 hours after transfection of 20,000 cells with 100 ng of each circularization reaction, followed by complete media replacement. Salivirus A GUT and Salivirus FHB exhibited the highest functional stability in HepG2 cells, and Salivirus N-J1 and Salivirus FHB produced the most stable expression in 1C1C7 cells (FIGS. 3A and 3B).

### Example 9

### Expression and Functional Stability by IRES in Jurkat Cells.

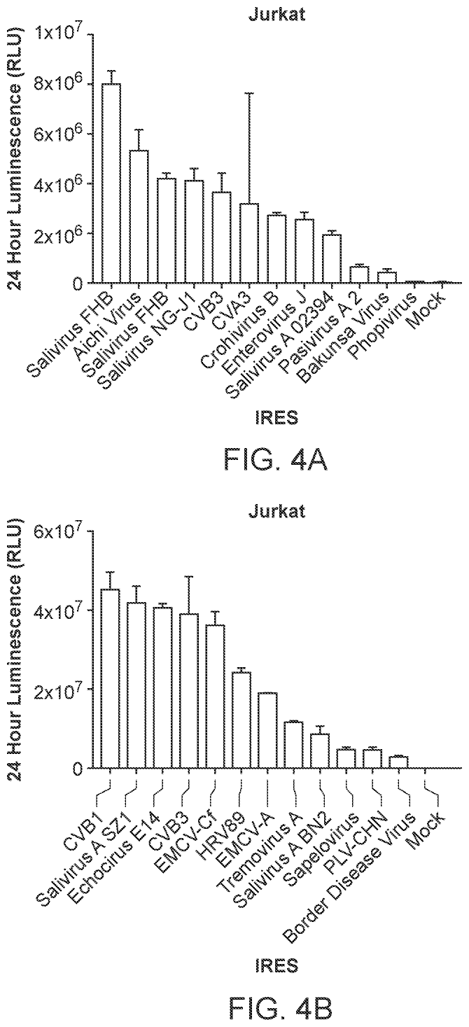

2 sets of constructs including anabaena intron/exon regions, a Gaussia luciferase expression sequence, and a subset of previously tested IRES were circularized. 60,000 Jurkat cells were electroporated with 1 μg of each circularization reaction. Luminescence from secreted Gaussia luciferase in supernatant was measured 24 hours after electroporation. A CVB3 IRES construct was included in both sets for comparison between sets and to previously defined IRES efficacy. CVB1 and Salivirus A SZ1 IRES constructs produced the most expression at 24 h. Data can be found in FIGS. 4A and 4B.

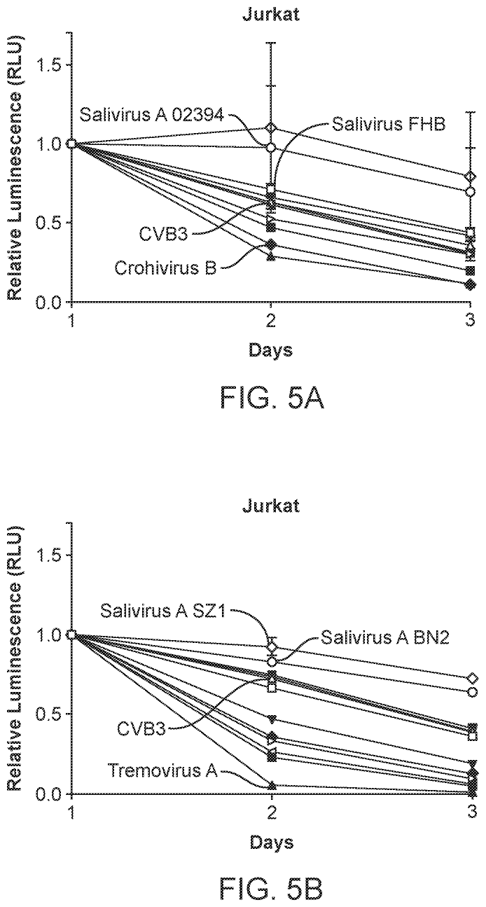

Functional stability of the IRES constructs in each round of electroporated Jurkat cells was measured over 3 days. Luminescence from secreted Gaussia luciferase in supernatant was measured every 24 hours after electroporation of 60,000 cells with 1 μg of each circularization reaction, followed by complete media replacement (FIGS. 5A and 5B).

Salivirus A SZ1 and Salivirus A BN2 IRES constructs had high functional stability compared to other constructs.

### Example 10

### Expression, Functional Stability, and Cytokine Release of Circular and Linear RNA in Jurkat Cells.

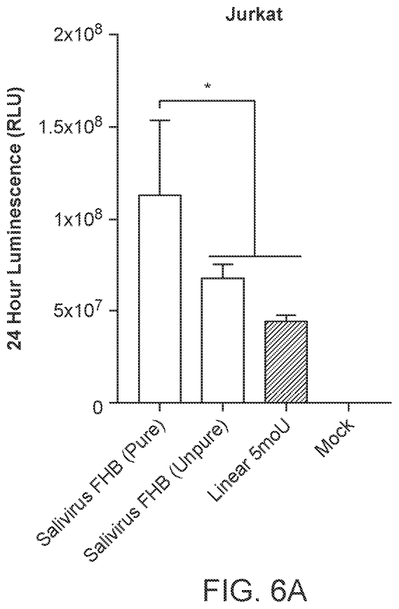

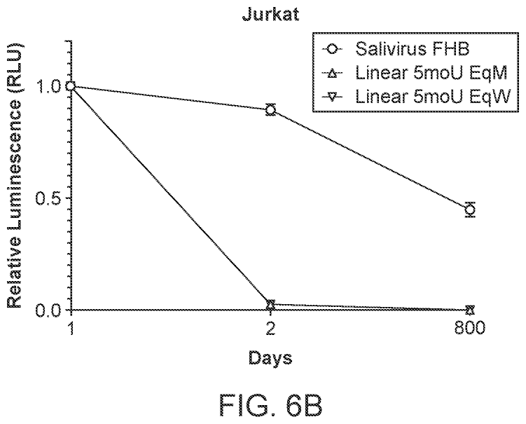

A construct including anabaena intron/exon regions, a Gaussia luciferase expression sequence, and a Salivirus FHB IRES was circularized. mRNA including a Gaussia luciferase expression sequence and a ˜150 nt polyA tail, and modified to replace 100% of uridine with 5-methoxy uridine (5moU) is commercially available and was purchased from Trilink. 5moU nucleotide modifications have been shown to improve mRNA stability and expression (Bioconjug Chem. 2016 Mar. 16; 27(3):849-53). Expression of modified mRNA, circularization reactions (unpure), and circRNA purified by size exclusion HPLC (pure) in Jurkat cells were measured and compared (FIG. 6A). Luminescence from secreted Gaussia luciferase in supernatant was measured 24 hours after electroporation of 60,000 cells with 1 μg of each RNA species.

Luminescence from secreted Gaussia luciferase in supernatant was measured every 24 hours after electroporation of 60,000 cells with 1 ug of each RNA species, followed by complete media replacement. A comparison of functional stability data of modified mRNA and circRNA in Jurkat cells over 3 days is in FIG. 6B.

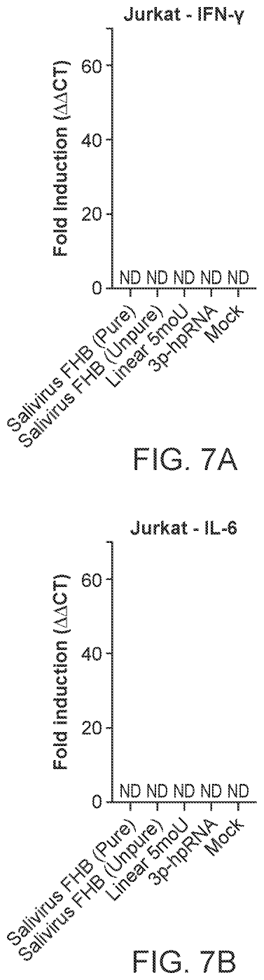

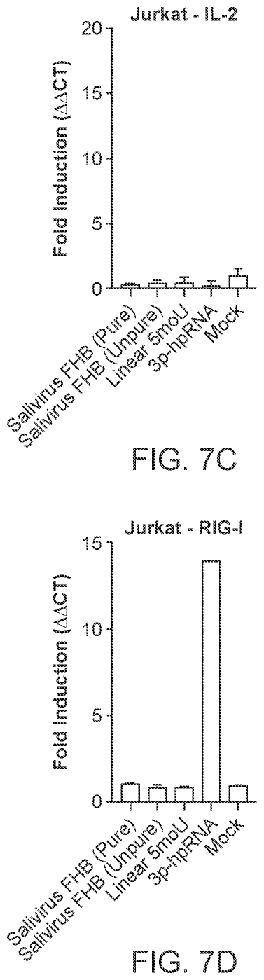

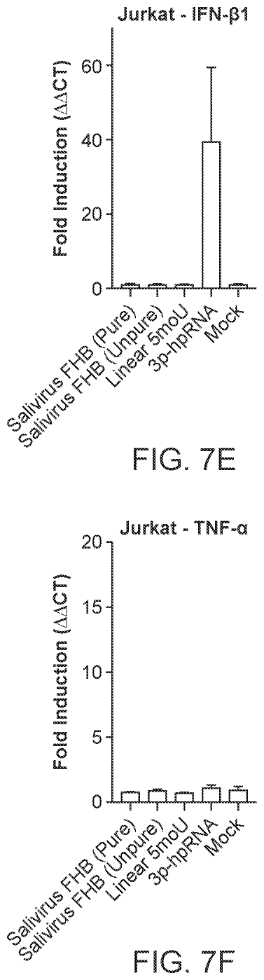

IFNγ (FIG. 7A), IL-6 (FIG. 7B), IL-2 (FIG. 7C), RIG-I (FIG. 7D), IFN-β1 (FIG. 7E), and TNFα (FIG. 7F) transcript induction was measured 18 hours after electroporation of 60,000 Jurkat cells with 1 μg of each RNA species described above and 3p-hpRNA (5′ triphosphate hairpin RNA, which is a known RIG-1 agonist).

### Example 11

### Expression of Circular and Linear RNA in Monocytes and Macrophages.

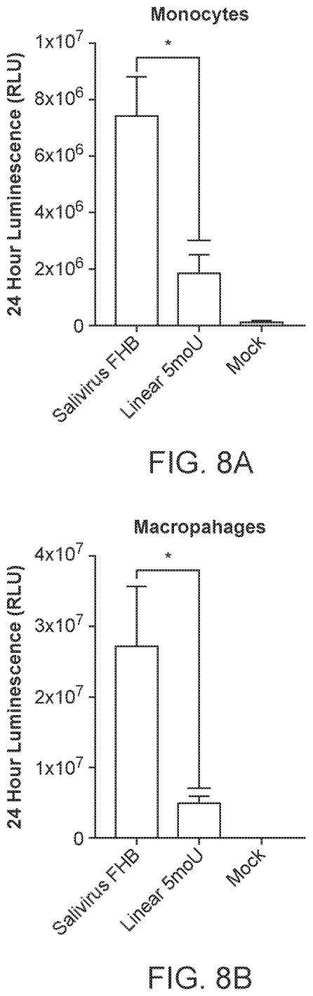

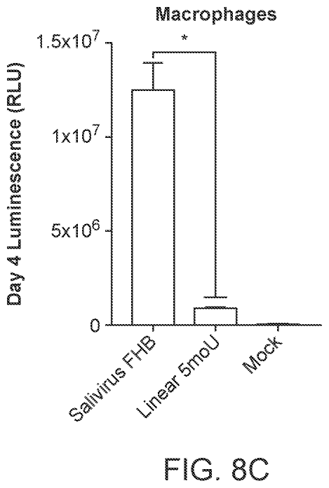

A construct including anabaena intron/exon regions, a Gaussia luciferase expression sequence, and a Salivirus FHB IRES was circularized. mRNA including a Gaussia luciferase expression sequence and a ˜150 nt polyA tail, and modified to replace 100% of uridine with 5-methoxy uridine (5moU) was purchased from Trilink. Expression of circular and modified mRNA was measured in human primary monocytes (FIG. 8A) and human primary macrophages (FIG. 8B). Luminescence from secreted Gaussia luciferase in supernatant was measured 24 hours after electroporation of 60,000 cells with 1 μg of each RNA species. Luminescence was also measured 4 days after electroporation of human primary macrophages with media changes every 24 hours (FIG. 8C). The difference in luminescence was statistically significant in each case (p<0.05).

### Example 12

### Expression and Functional Stability by IRES in Primary T Cells.

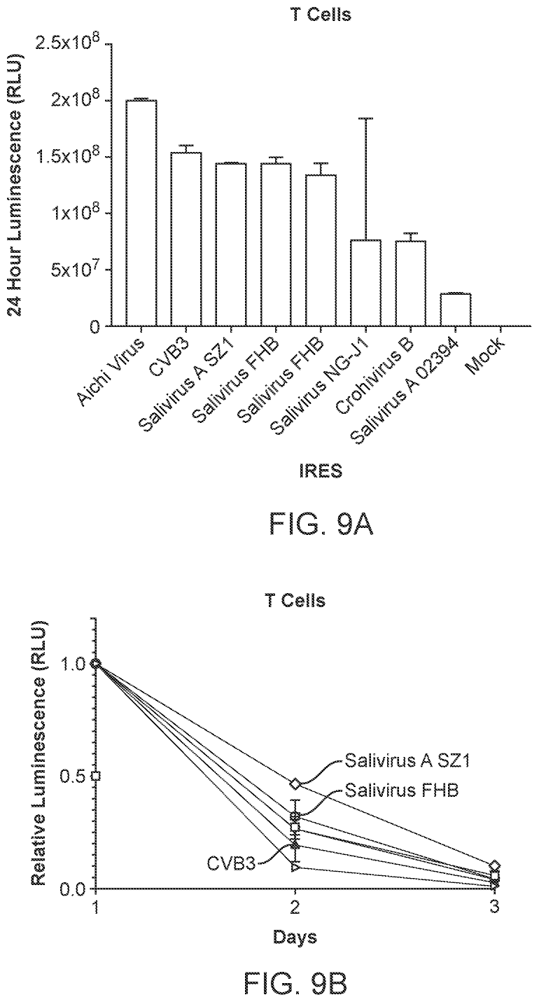

Constructs including anabaena intron/exon regions, a Gaussia luciferase expression sequence, and a subset of previously tested IRES were circularized and reaction products were purified by size exclusion HPLC. 150,000 primary human CD3+ T cells were electroporated with 1 μg of each circRNA. Luminescence from secreted Gaussia luciferase in supernatant was measured 24 hours after electroporation (FIG. 9A). Aichi Virus and CVB3 IRES constructs had the most expression at 24 hours.

Luminescence was also measured every 24 hours after electroporation for 3 days in order to compare functional stability of each construct (FIG. 9B). The construct with a Salivirus A SZ1 IRES was the most stable.

### Example 13

### Expression and Functional Stability of Circular and Linear RNA in Primary T Cells and PBMCs.

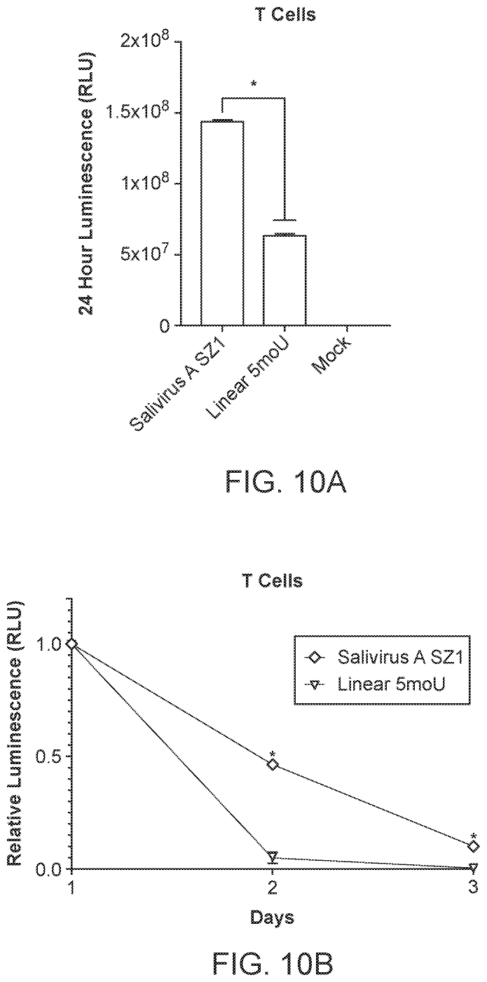

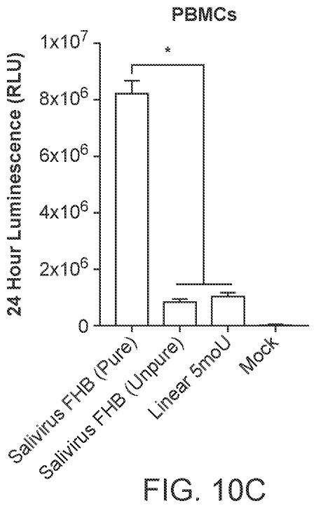

Constructs including anabaena intron/exon regions, a Gaussia luciferase expression sequence, and a Salivirus A SZ 1 IRES or Salivirus FHB IRES were circularized. mRNA including a Gaussia luciferase expression sequence and a ˜150 nt polyA tail, and modified to replace 100% of uridine with 5-methoxy uridine (5moU) and was purchased from Trilink. Expression of Salivirus A SZ1 IRES HPLC purified circular and modified mRNA was measured in human primary CD3+ T cells. Expression of Salivirus FHB HPLC purified circular, unpurified circular and modified mRNA was measured in human PBMCs. Luminescence from secreted Gaussia luciferase in supernatant was measured 24 hours after electroporation of 150,000 cells with 1 μg of each RNA species. Data for primary human T cells is in FIGS. 10A and 10B, and data for PBMCs is in FIG. 10C. The difference in expression between the purified circular RNA and unpurified circular RNA or linear RNA was significant in each case (p<0.05).

Luminescence from secreted Gaussia luciferase in primary T cell supernatant was measured every 24 hours after electroporation over 3 days in order to compare construct functional stability. Data is shown in FIG. JOB. The difference in relative luminescence from the day 1 measurement between purified circular RNA and linear RNA was significant at both day 2 and day 3 for primary T cells.

### Example 14

### Circularization Efficiency by Permutation Site in Anabaena Intron.

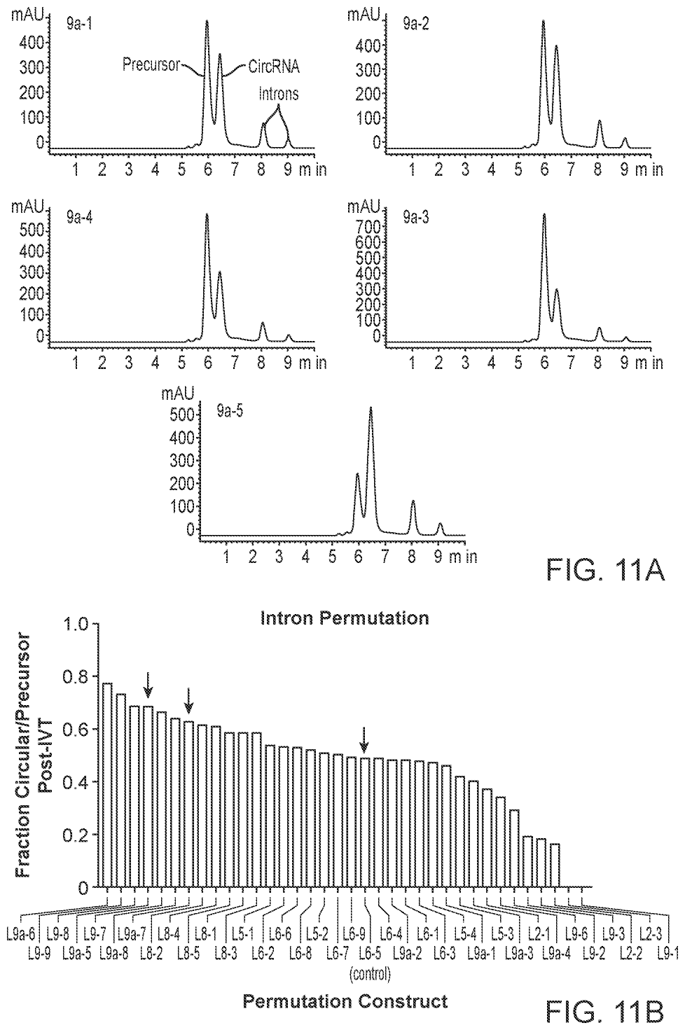

RNA constructs including a CVB3 IRES, a Gaussia luciferase expression sequence, anabaena intron/exon regions, spacers, internal duplex forming regions, and homology arms were produced. Circularization efficiency of constructs using the traditional anabaena intron permutation site and 5 consecutive permutations sites in P9 was measured by HPLC. HPLC chromatograms for the 5 consecutive permutation sites in P9 are shown in FIG. 11A.

Circularization efficiency was measured at a variety of permutation sites. Circularization efficiency is defined as the area under the HPLC chromatogram curve for each of: circRNA/(circRNA+precursor RNA). Ranked quantification of circularization efficiency at each permutation site is in FIG. 11B. 3 permutation sites (indicated in FIG. 11B) were selected for further investigation.

Circular RNA in this example was circularized by in vitro transcription (IVT) then purified via spin column. Circularization efficiency for all constructs would likely be higher if the additional step of incubation with Mg²⁺ and guanosine nucleotide were included; however, removing this step allowed for comparison between, and optimization of, circular RNA constructs. This level of optimization is especially useful for maintaining high circularization efficiency with large RNA constructs, such as those encoding chimeric antigen receptors.

### Example 15

### Circularization Efficiency of Alternative Introns.

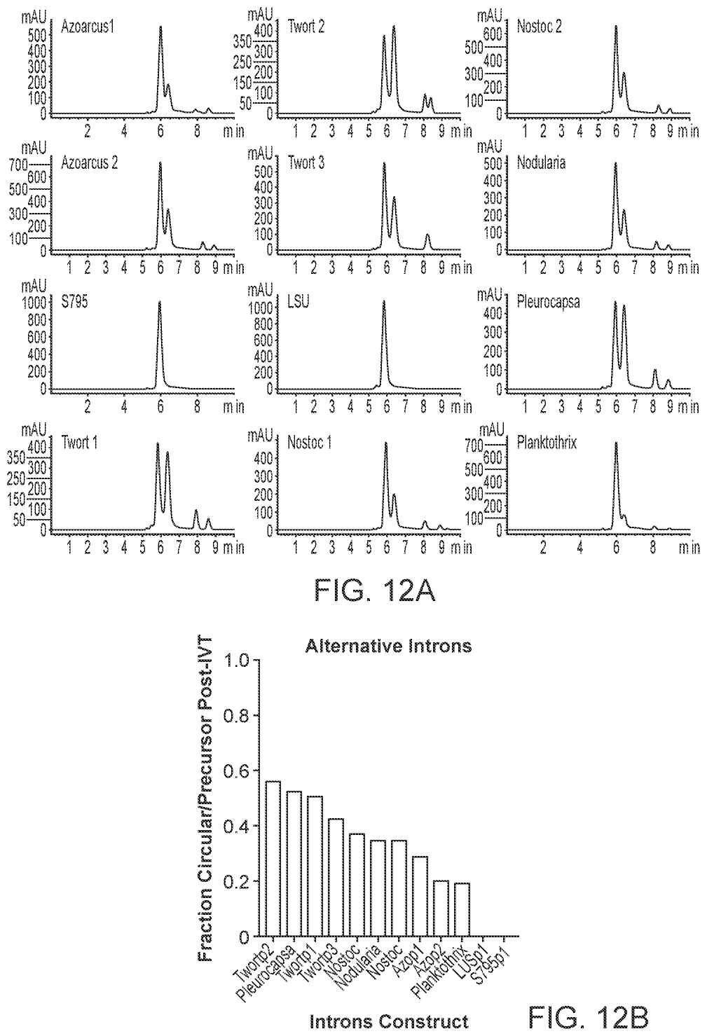

Precursor RNA containing a permuted group 1 intron of variable species origin or permutation site and several constant elements including: a CVB3 IRES, a Gaussia luciferase expression sequence, spacers, internal duplex forming regions, and homology arms were created. Circularization data can be found in FIG. 12. FIG. 12A shows chromatograms resolving precursor, CircRNA and introns. FIG. 12B provides ranked quantification of circularization efficiency, based on the chromatograms shown in FIG. 12A, as a function of intron construct.

Circular RNA in this example was circularized by in vitro transcription (IVT) then spin column purification. Circularization efficiency for all constructs would likely be higher if the additional step of incubation with Mg²⁺ and guanosine nucleotide were included; however, removing this step allows for comparison between, and optimization of, circular RNA constructs. This level of optimization is especially useful for maintaining high circularization efficiency with large RNA constructs, such as those encoding chimeric antigen receptors.

### Example 16

### Circularization Efficiency by Homology Arm Presence or Length.

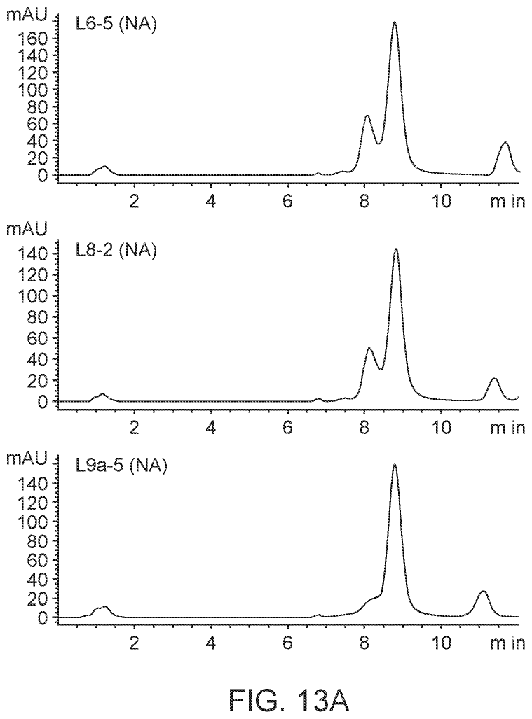

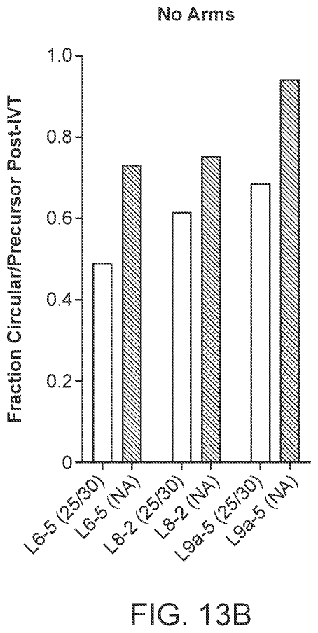

RNA constructs including a CVB3 IRES, a Gaussia luciferase expression sequence, anabaena intron/exon regions, spacers, and internal duplex forming regions were produced. Constructs representing 3 anabaena intron permutation sites were tested with 30 nt, 25% GC homology arms or without homology arms (“NA”). These constructs were allowed to circularize without the step of incubation with Mg²⁺. Circularization efficiency was measured and compared. Data can be found in FIG. 13. Circularization efficiency was higher for each construct lacking homology arms. FIG. 13A provides ranked quantification of circularization efficiency; FIG. 13B provides chromatograms resolving precursor, circRNA and introns.

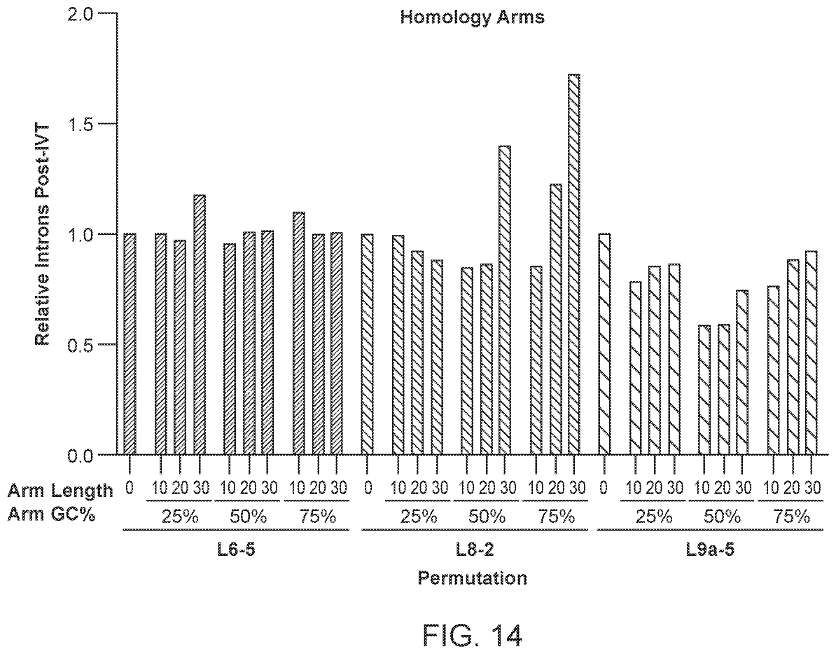

For each of the 3 permutation sites, constructs were created with 10 nt, 20 nt, and 30 nt arm length and 25%, 50%, and 75% GC. Splicing efficiency of these constructs was measured and compared to constructs without homology arms (FIG. 14). Splicing efficiency is defined as the proportion of free introns relative to the total RNA in the splicing reaction.

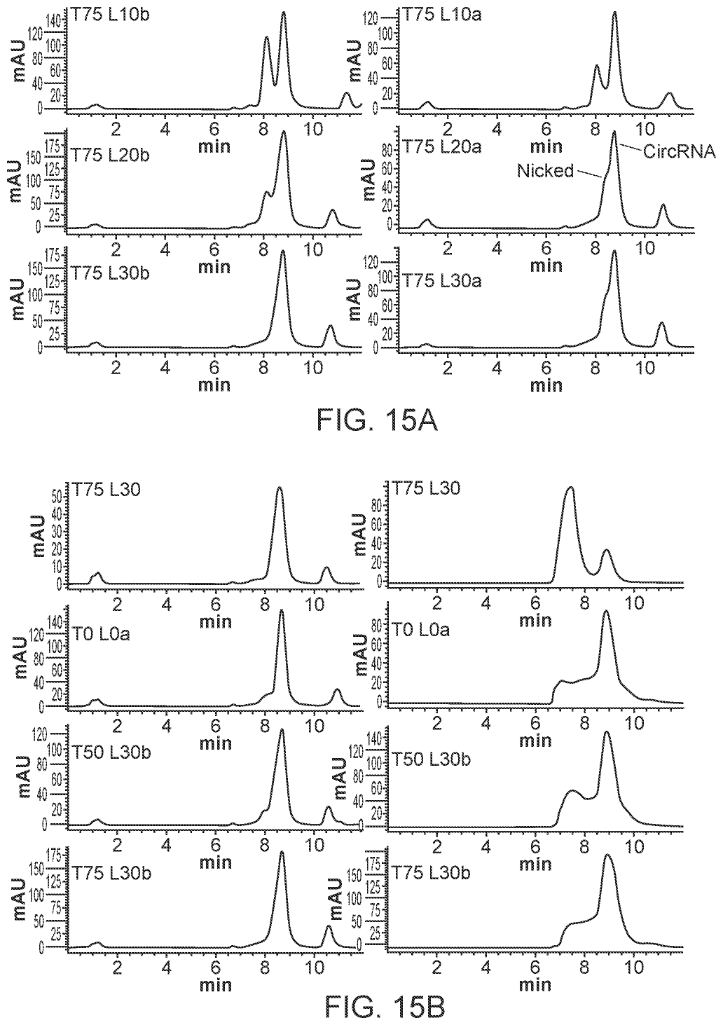

FIG. 15 A (left) contains HPLC chromatograms showing the contribution of strong homology arms to improved splicing efficiency. Top left: 75% GC content, 10 nt homology arms. Center left: 75% GC content, 20 nt homology arms. Bottom left: 75% GC content, 30 nt homology arms.

FIG. 15 A (right) shows HPLC chromatograms indicating increased splicing efficiency paired with increased nicking, appearing as a shoulder on the circRNA peak. Top right: 75% GC content, 10 nt homology arms. Center right: 75% GC content, 20 nt homology arms. Bottom right: 75% GC content, 30 nt homology arms.

FIG. 15 B (left) shows select combinations of permutation sites and homology arms hypothesized to demonstrate improved circularization efficiency.

FIG. 15 B (right) shows select combinations of permutation sites and homology arms hypothesized to demonstrate improved circularization efficiency, treated with E. coli polyA polymerase.

Circular RNA in this example was circularized by in vitro transcription (IVT) then spin-column purified. Circularization efficiency for all constructs would likely be higher if an additional Mg²⁺ incubation step with guanosine nucleotide were included; however, removing this step allowed for comparison between, and optimization of, circular RNA constructs. This level of optimization is especially useful for maintaining high circularization efficiency with large RNA constructs, such as those encoding chimeric antigen receptors.

### Example 17

### Circular RNA Encoding Chimeric Antigen Receptors.

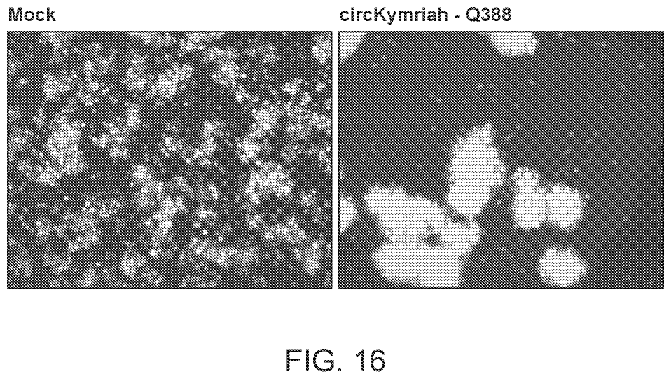

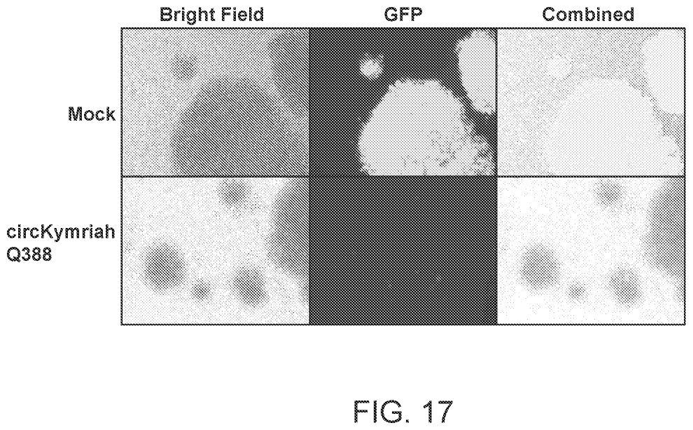

Constructs including anabaena intron/exon regions, a Kymriah chimeric antigen receptor (CAR) expression sequence, and a CVB3 IRES were circularized. 100,000 human primary CD3+ T cells were electroporated with 500ng of circRNA and co-cultured for 24 hours with Raji cells stably expressing GFP and firefly luciferase. Effector to target ratio (E:T ratio) 0.75:1. 100,000 human primary CD3+ T cells were mock electroporated and co-cultured as a control (FIG. 16).

Sets of 100,000 human primary CD3+ T cells were mock electroporated or electroporated with 1 μg of circRNA then co-cultured for 48 hours with Raji cells stably expressing GFP and firefly luciferase. E:T ratio 10:1 (FIG. 17).

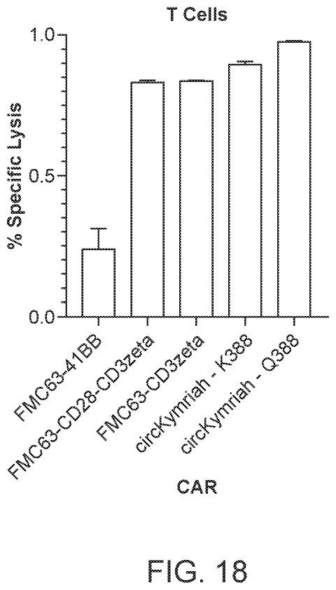

Quantification of specific lysis of Raji target cells was determined by detection of firefly luminescence (FIG. 18). 100,000 human primary CD3+ T cells either mock electroporated or electroporated with circRNA encoding different CAR sequences were co-cultured for 48 hours with Raji cells stably expressing GFP and firefly luciferase. % Specific lysis defined as 1-[CAR condition luminescence]/[mock condition luminescence]. E:T ratio 10:1.

### Example 18

### Expression and Functional Stability of Circular and Linear RNA in Jurkat Cells and Resting Human T Cells.

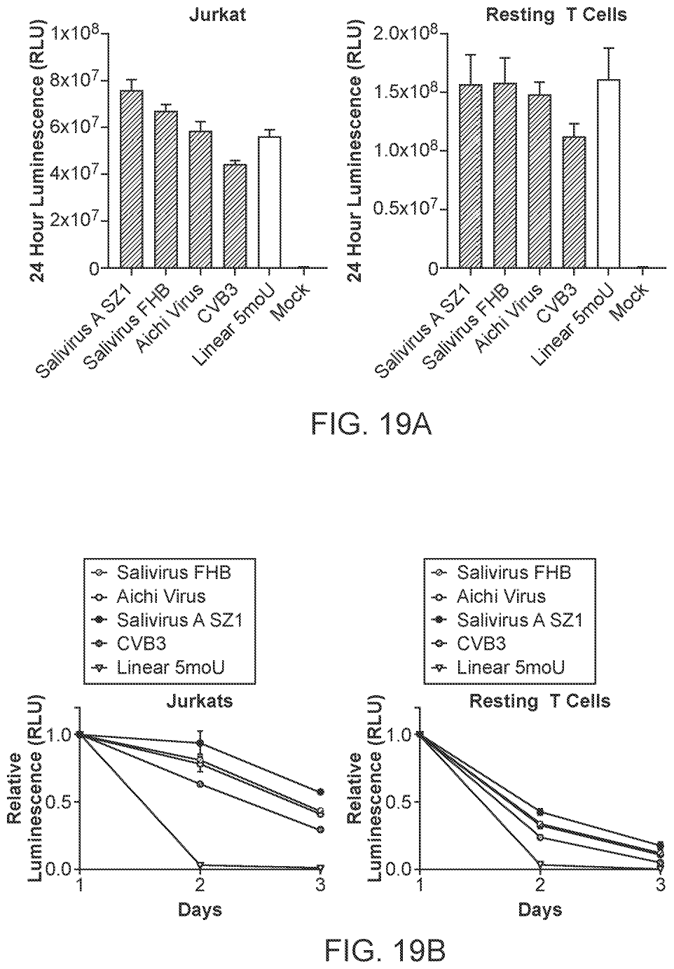

Constructs including anabaena intron/exon regions, a Gaussia luciferase expression sequence, and a subset of previously tested IRES were circularized and reaction products were purified by size exclusion HPLC. 150,000 Jurkat cells were electroporated with 1 μg of circular RNA or 5moU-mRNA. Luminescence from secreted Gaussia luciferase in supernatant was measured 24 hours after electroporation (FIG. 19A left). 150,000 resting primary human CD3+ T cells (10 days post-stimulation) were electroporated with 1 μg of circular RNA or 5moU-mRNA. Luminescence from secreted Gaussia luciferase in supernatant was measured 24 hours after electroporation (FIG. 19A right).

Luminescence from secreted Gaussia luciferase in supernatant was measured every 24 hours after electroporation, followed by complete media replacement. Functional stability data is shown in FIG. 19B. Circular RNA had more functional stability than linear RNA in each case, with a more pronounced difference in Jurkat cells.

### Example 19

IFN-β1, RIG-I, IL-2, IL-6, IFNγ, and TNFα Transcript Induction of Cells Electroporated with Linear RNA or Varying Circular RNA Constructs.

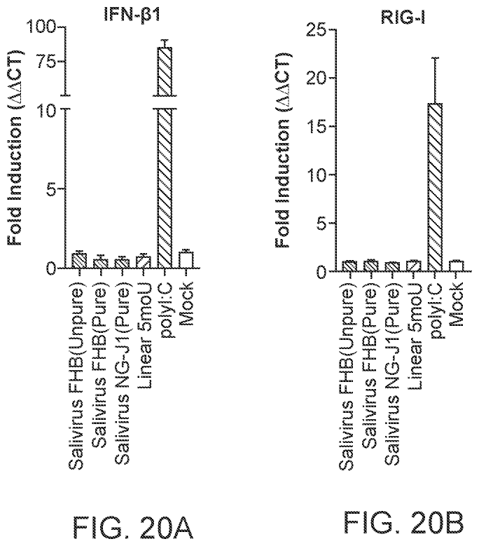

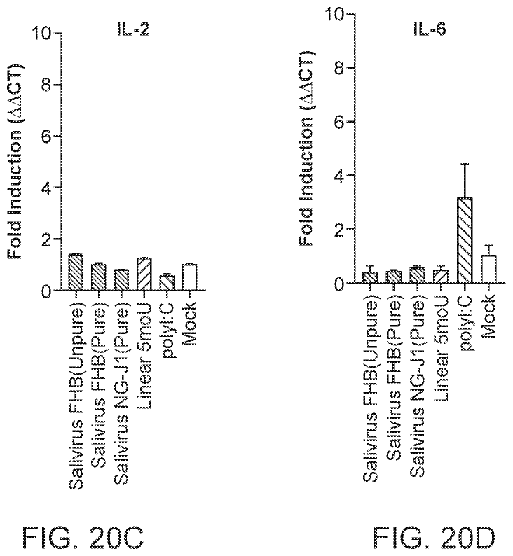

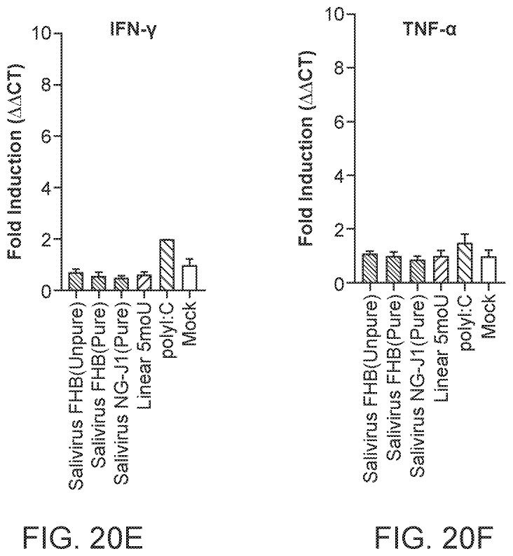

Constructs including anabaena intron/exon regions, a Gaussia luciferase expression sequence, and a subset of previously tested IRES were circularized and reaction products were purified by size exclusion HPLC. 150,000 CD3+ human T cells were electroporated with 1 μg of circular RNA, 5moU-mRNA, or immunostimulatory positive control poly inosine:cytosine. IFN-β1 (FIG. 20A), RIG-I (FIG. 20B), IL-2 (FIG. 20C), IL-6 (FIG. 20D), IFN-γ (FIG. 20E), and TNF-α (FIG. 20F) transcript induction was measured 18 hours after electroporation.

### Example 20

Specific Lysis of Target Cells and IFNγ Transcript Induction by CAR Expressing Cells Electroporated with Different Amounts of Circular or Linear RNA; Specific Lysis of Target and Non-Target Cells by CAR Expressing Cells at Different E:T Ratios.

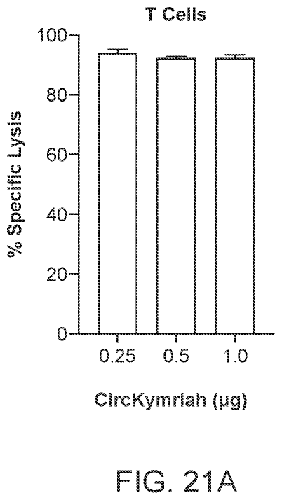

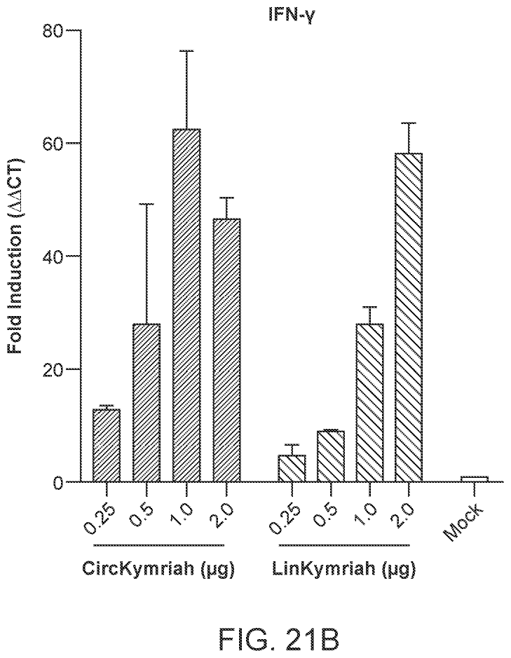

Constructs including anabaena intron/exon regions, an anti-CD19 CAR expression sequence, and a CVB3 IRES were circularized and reaction products were purified by size exclusion HPLC. 150,000 human primary CD3+ T cells either mock electroporated or electroporated with different quantities of circRNA encoding an anti-CD19 CAR sequence were co-cultured for 12 hours with Raji cells stably expressing GFP and firefly luciferase at an E:T ratio of 2:1. Specific lysis of Raji target cells was determined by detection of firefly luminescence (FIG. 21A). % Specific lysis was defined as 1−[CAR condition luminescence]/[mock condition luminescence]. IFNγ transcript induction was measured 24 hours after electroporation (FIG. 21B).

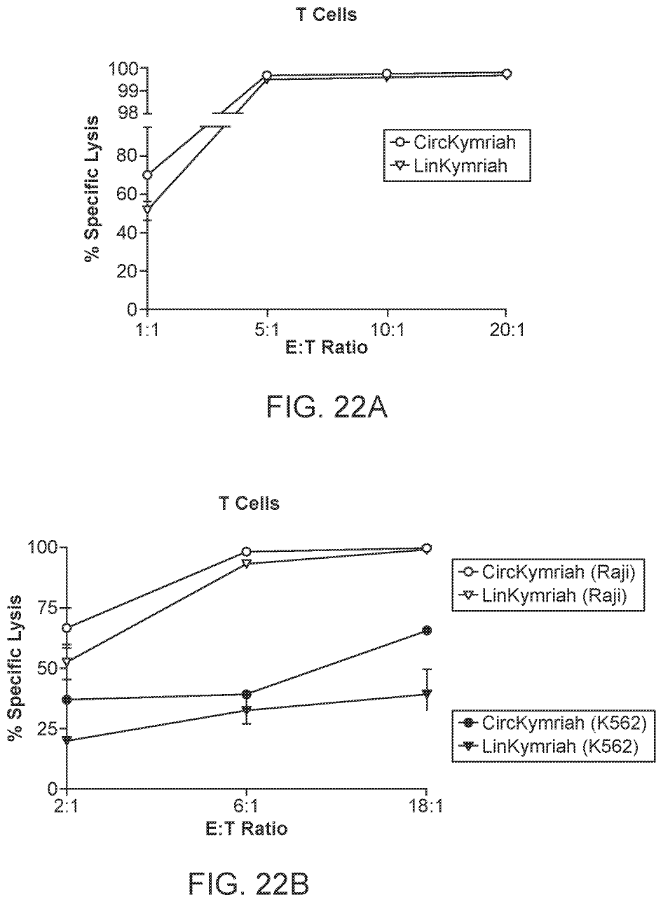

150,000 human primary CD3+ T cells were either mock electroporated or electroporated with 500ng circRNA or m1W-mRNA encoding an anti-CD19 CAR sequence, then co-cultured for 24 hours with Raji cells stably expressing firefly luciferase at different E:T ratios. Specific lysis of Raji target cells was determined by detection of firefly luminescence (FIG. 22A). Specific lysis was defined as 1−[CAR condition luminescence]/[mock condition luminescence].

CAR expressing T cells were also co-cultured for 24 hours with Raji or K562 cells stably expressing firefly luciferase at different E:T ratios. Specific lysis of Raji target cells or K562 non-target cells was determined by detection of firefly luminescence (FIG. 22B). % Specific lysis is defined as 1−[CAR condition luminescence]/[mock condition luminescence].

### Example 21

Specific Lysis of Target Cells by T Cells Electroporated with Circular RNA or Linear RNA Encoding a CAR.

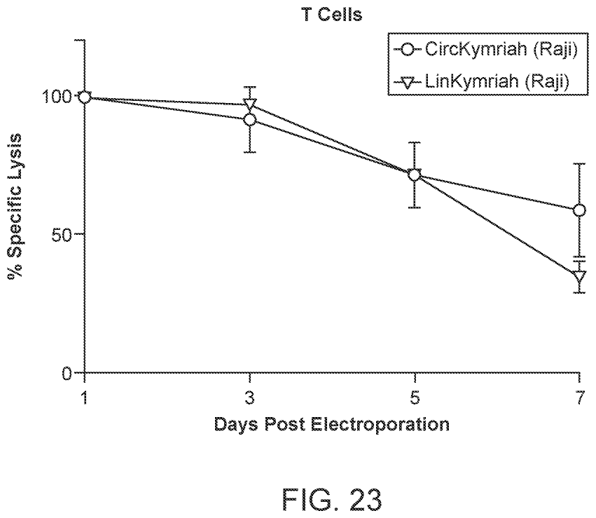

Constructs including anabaena intron/exon regions, an anti-CD19 CAR expression sequence, and a CVB3 IRES were circularized and reaction products were purified by size exclusion HPLC. Human primary CD3+ T cells were electroporated with 500 ng of circular RNA or an equimolar quantity of m1W-mRNA, each encoding a CD19-targeted CAR. Raji cells were added to CAR-T cell cultures over 7 days at an E:T ratio of 10:1. % Specific lysis was measured for both constructs at 1, 3, 5, and 7 days (FIG. 23).

### Example 22

### Specific Lysis of Raji Cells by T Cells Expressing an Anti-CD19 CAR or an Anti-BCMA CAR.

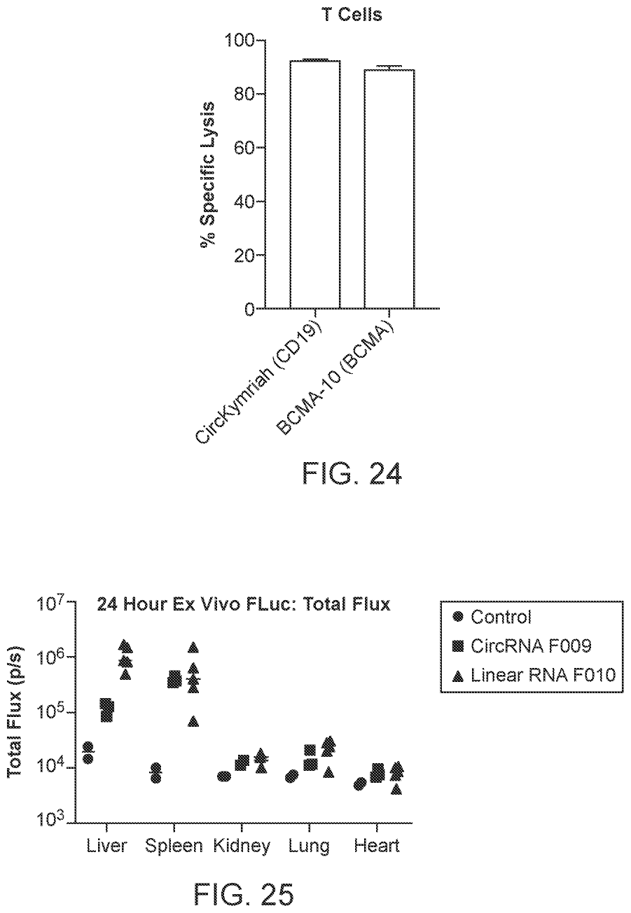

Constructs including anabaena intron/exon regions, anti-CD19 or anti-BCMA CAR expression sequence, and a CVB3 IRES were circularized and reaction products were purified by size exclusion HPLC. 150,000 primary human CD3+ T cells were electroporated with 500ng of circRNA, then were co-cultured with Raji cells at an E:T ratio of 2:1. % Specific lysis was measured 12 hours after electroporation (FIG. 24).

### Example 23

### Expression, Functional Stability, and Cytokine Transcript Induction of Circular and Linear RNA Expressing Antigens.

Constructs including one or more antigen expression sequences are circularized and reaction products are purified by size exclusion HPLC. Antigen presenting cells are electroporated with circular RNA or mRNA.

In vitro antigen production is measured via ELISA. Optionally, antigen production is measured every 24 hours after electroporation. Cytokine transcript induction or release is measured 18 hours after electroporation of antigen presenting cells with circular or linear RNA encoding antigens. The tested cytokines may include IFN-β1, RIG-I, IL-2, IL-6, IFNγ, RANTES, and TNFα.

In vitro antigen production and cytokine induction are measured using purified circRNA, purified circRNA plus antisense circRNA, and unpurified circRNA in order to find the ratio that best preserves expression and immune stimulation.

### Example 24

### In Vivo Antigen and Antibody Expression in Animal Models.

To assess the ability of antigen encoding circRNAs to facilitate antigen expression and antibody production in vivo, escalating doses of RNA encoding one or more antigens is introduced into mice via intramuscular injection.

Mice are injected once, blood collected after 28 days, then injected again, with blood collected 14 days thereafter. Neutralizing antibodies against antigen of interest is measured via ELISA.

### Example 25

### Protection Against Infection.

To assess the ability of antigen encoding circRNAs to protect against or cure an infection, RNA encoding one or more antigens of a virus (such as influenza) is introduced into mice via intramuscular injection.

Mice receive an initial injection and boost injections of circRNA encoding one or more antigens. Protection from a virus such as influenza is determined by weight loss and mortality over 2 weeks.

### Example 26

### Example 26A: Synthesis of Compounds

Synthesis of representative ionizable lipids of the invention are described in PCT applications PCT/US2016/052352, PCT/US2016/068300, PCT/US2010/061058, PCT/US2018/058555, PCT/US2018/053569, PCT/US2017/028981, PCT/US2019/025246, PCT/US2018/035419, PCT/US2019/015913, and US applications with publication numbers 20190314524, 20190321489, and 20190314284, the contents of each of which are incorporated herein by reference in their entireties.

### Example 26B: Synthesis of Compounds

Synthesis of representative ionizable lipids of the invention are described in US patent publication number US20170210697A 1, the contents of which is incorporated herein by reference in its entirety.

### Example 27

### Protein Expression by Organ

Circular or linear RNA encoding FLuc was generated and loaded into transfer vehicles with the following formulation: 50% ionizable Lipid 10b-15 represented by

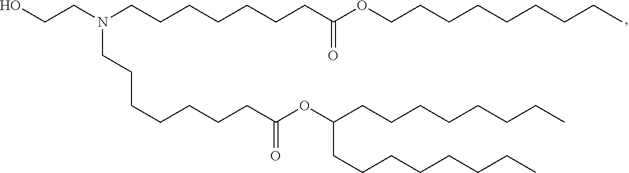 [SMILES: CCCCCCCCCOC(=O)CCCCCCCN(CCO)CCCCCCCC(=O)OC(CCCCCCCC)CCCCCCCC]

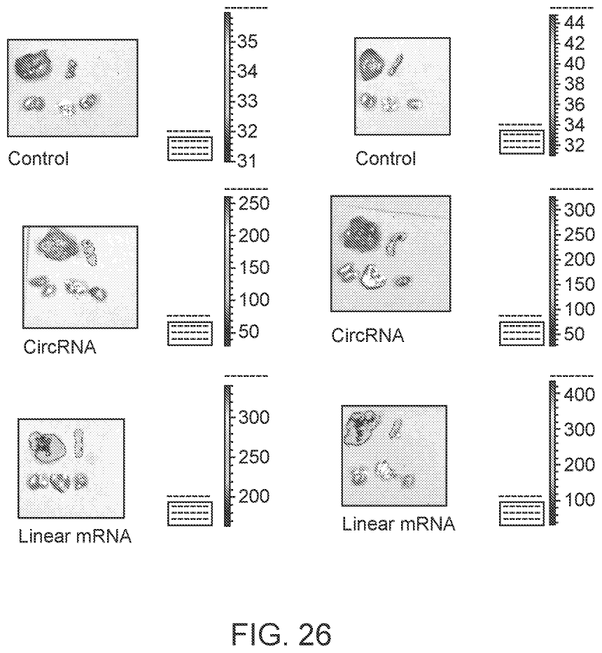

10% DSPC, 1.5% PEG-DMG, 38.5% cholesterol. CD-1 mice were dosed at 0.2 mg/kg and luminescence was measured at 6 hours (live IVIS) and 24 hours (live IVIS and ex vivo IVIS). Total Flux (photons/second over a region of interest) of the liver, spleen, kidney, lung, and heart was measured (FIGS. 25 and 26).

### Example 28

### Distribution of Expression in the Spleen

Circular or linear RNA encoding GFP is generated and loaded into transfer vehicles with the following formulation: 50% ionizable Lipid 10b-15 represented by

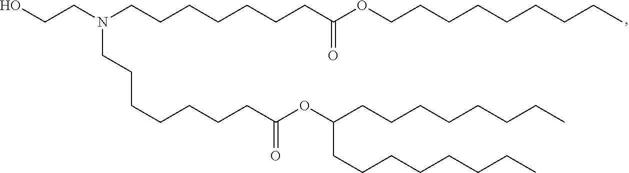 [SMILES: CCCCCCCCCOC(=O)CCCCCCCN(CCO)CCCCCCCC(=O)OC(CCCCCCCC)CCCCCCCC]

10% DSPC, 1.5% PEG-DMG, 38.5% cholesterol. The formulation is administered to CD-1 mice. Flow cytometry is run on spleen cells to determine the distribution of expression across cell types.

### Example 29

### Example 29A: Production of Nanoparticle Compositions

In order to investigate safe and efficacious nanoparticle compositions for use in the delivery of circular RNA to cells, a range of formulations are prepared and tested. Specifically, the particular elements and ratios thereof in the lipid component of nanoparticle compositions are optimized.

Nanoparticles can be made in a 1 fluid stream or with mixing processes such as microfluidics and T-junction mixing of two fluid streams, one of which contains the circular RNA and the other has the lipid components.

Lipid compositions are prepared by combining an ionizable lipid, optionally a helper lipid (such as DOPE, DSPC, or oleic acid obtainable from Avanti Polar Lipids, Alabaster, AL), a PEG lipid (such as 1,2-dimyristoyl-sn-glycerol methoxypolyethylene glycol, also known as PEG-DMG, obtainable from Avanti Polar Lipids, Alabaster, AL), and a structural lipid such as cholesterol at concentrations of about, e.g., 40 or 50 mM in a solvent, e.g., ethanol. Solutions should be refrigerated for storage at, for example, −20° C. Lipids are combined to yield desired molar ratios (see, for example, Tables 11a and 11b below) and diluted with water and ethanol to a final lipid concentration of e.g., between about 5.5 mM and about 25 mM.

TABLE 11a

| Formulation | |
|---|---|
| number | Description |
| 1 | Aliquots of 50 mg/mL ethanolic solutions of C12-200, DOPE, Chol and DMG- |
| | PEG2K (40:30:25:5) are mixed and diluted with ethanol to 3 mL final volume. |
| | Separately, an aqueous buffered solution (10 mM citrate/150 mM NaCl, pH 4.5) of |
| | circRNA is prepared from a 1 mg/mL stock. The lipid solution is injected rapidly |
| | into the aqueous circRNA solution and shaken to yield a final suspension in 20% |
| | ethanol. The resulting nanoparticle suspension is filtered, diafiltrated with 1 × PBS |
| | (pH 7.4), concentrated and stored at 2-8° C. |
| 2 | Aliquots of 50 mg/mL ethanolic solutions of DODAP, DOPE, cholesterol and DMG- |
| | PEG2K (18:56:20:6) are mixed and diluted with ethanol to 3 mL final volume. |
| | Separately, an aqueous buffered solution (10 mM citrate/150 mM NaCl, pH 4.5) of |
| | EPO circRNA is prepared from a 1 mg/mL stock. The lipid solution is injected |
| | rapidly into the aqueous circRNA solution and shaken to yield a final suspension in |
| | 20% ethanol. The resulting nanoparticle suspension is filtered, diafiltrated with |
| | 1 × PBS (pH 7.4), concentrated and stored at 2-8° C. Final concentration = 1.35 |
| | mg/mL EPO circRNA (encapsulated). Zave = 75.9 nm (Dv(50) = 57.3 nm; |
| | Dv(90) = 92.1 nm). |
| 3 | Aliquots of 50 mg/mL ethanolic solutions of HGT4003, DOPE, cholesterol and |
| | DMG-PEG2K (50:25:20:5) are mixed and diluted with ethanol to 3 mL final volume. |
| | Separately, an aqueous buffered solution (10 mM citrate/150 mM NaCl, pH 4.5) of |
| | circRNA is prepared from a 1 mg/mL stock. The lipid solution is injected rapidly |
| | into the aqueous circRNA solution and shaken to yield a final suspension in 20% |
| | ethanol. The resulting nanoparticle suspension is filtered, diafiltrated with 1 × PBS |
| | (pH 7.4), concentrated and stored at 2-8° C. |
| 4 | Aliquots of 50 mg/mL ethanolic solutions of ICE, DOPE and DMG-PEG2K |
| | (70:25:5) are mixed and diluted with ethanol to 3 mL final volume. Separately, an |
| | aqueous buffered solution (10 mM citrate/150 mM NaCl, pH 4.5) of circRNA is |
| | prepared from a 1 mg/mL stock. The lipid solution is injected rapidly into the |
| | aqueous circRNA solution and shaken to yield a final suspension in 20% ethanol. |
| | The resulting nanoparticle suspension is filtered, diafiltrated with 1 × PBS (pH 7.4), |
| | concentrated and stored at 2-8° C. |
| 5 | Aliquots of 50 mg/mL ethanolic solutions of HGT5000, DOPE, cholesterol and |
| | DMG-PEG2K (40:20:35:5) are mixed and diluted with ethanol to 3 mL final volume. |
| | Separately, an aqueous buffered solution (10 mM citrate/150 mM NaCl, pH 4.5) of |
| | EPO circRNA is prepared from a 1 mg/mL stock. The lipid solution is injected |
| | rapidly into the aqueous circRNA solution and shaken to yield a final suspension in |
| | 20% ethanol. The resulting nanoparticle suspension is filtered, diafiltrated with |
| | 1 × PBS (pH 7.4), concentrated and stored at 2-8° C. Final concentration = 1.82 |
| | mg/mL EPO mRNA (encapsulated). Zave = 105.6 nm (Dv(50) = 53.7 nm; |
| | Dv(90) = 157 nm). |
| 6 | Aliquots of 50 mg/mL ethanolic solutions of HGT5001, DOPE, cholesterol and |
| | DMG-PEG2K (40:20:35:5) are mixed and diluted with ethanol to 3 mL final volume. |
| | Separately, an aqueous buffered solution (10 mM citrate/150 mM NaCl, pH 4.5) of |
| | EPO circRNA is prepared from a 1 mg/mL stock. The lipid solution is injected |
| | rapidly into the aqueous circRNA solution and shaken to yield a final suspension in |
| | 20% ethanol. The resulting nanoparticle suspension is filtered, diafiltrated with |
| | 1 × PBS (pH 7.4), concentrated and stored at 2-8° C. |
| 7 | Aliquots of 50 mg/mL ethanolic solutions of HGT5001, DOPE, cholesterol and |
| | DMG-PEG2K (35:16:46.5:2.5) are mixed and diluted with ethanol to 3 mL final |
| | volume. Separately, an aqueous buffered solution (10 mM citrate/150 mM NaCl, pH |
| | 4.5) of EPO circRNA is prepared from a 1 mg/mL stock. The lipid solution is injected |
| | rapidly into the aqueous circRNA solution and shaken to yield a final suspension in |
| | 20% ethanol. The resulting nanoparticle suspension is filtered, diafiltrated with |
| | 1 × PBS (pH 7.4), concentrated and stored at 2-8° C. |
| 8 | Aliquots of 50 mg/mL ethanolic solutions of HGT5001, DOPE, cholesterol and |
| | DMG-PEG2K (40:10:40:10) are mixed and diluted with ethanol to 3 mL final |
| | volume. Separately, an aqueous buffered solution (10 mM citrate/150 mM NaCl, pH |
| | 4.5) of EPO circRNA is prepared from a 1 mg/mL stock. The lipid solution is injected |
| | rapidly into the aqueous circRNA solution and shaken to yield a final suspension in |
| | 20% ethanol. The resulting nanoparticle suspension is filtered, diafiltrated with |
| | 1 × PBS (pH 7.4), concentrated and stored at 2-8° C. |

In some embodiments, transfer vehicle has a formulation as described in Table 11a.

TABLE 11b

| Composition | |
|---|---|
| (mol %) | Components |
| 40:20:38.5:1.5 | Compound:Phospholipid:Phytosterol*:PEG- |
| | DMG |
| 45:15:38.5:1.5 | Compound:Phospholipid:Phytosterol*:PEG- |
| | DMG |
| 50:10:38.5:1.5 | Compound:Phospholipid:Phytosterol*:PEG- |
| | DMG |
| 55:5:38.5:1.5 | Compound:Phospholipid:Phytosterol*:PEG- |
| | DMG |
| 60:5:33.5:1.5 | Compound:Phospholipid:Phytosterol*:PEG- |
| | DMG |
| 45:20:33.5:1.5 | Compound:Phospholipid:Phytosterol*:PEG- |
| | DMG |
| 50:20:28.5:1.5 | Compound:Phospholipid:Phytosterol*:PEG- |
| | DMG |
| 55:20:23.5:1.5 | Compound:Phospholipid:Phytosterol*:PEG- |
| | DMG |
| 60:20:18.5:1.5 | Compound:Phospholipid:Phytosterol*:PEG- |
| | DMG |
| 40:15:43.5:1.5 | Compound:Phospholipid:Phytosterol*:PEG- |
| | DMG |
| 50:15:33.5:1.5 | Compound:Phospholipid:Phytosterol*:PEG- |
| | DMG |
| 55:15:28.5:1.5 | Compound:Phospholipid:Phytosterol*:PEG- |
| | DMG |
| 60:15:23.5:1.5 | Compound:Phospholipid:Phytosterol*:PEG- |
| | DMG |
| 40:10:48.5:1.5 | Compound:Phospholipid:Phytosterol*:PEG- |
| | DMG |
| 45:10:43.5:1.5 | Compound:Phospholipid:Phytosterol*:PEG- |
| | DMG |
| 55:10:33.5:1.5 | Compound:Phospholipid:Phytosterol*:PEG- |
| | DMG |
| 60:10:28.5:1.5 | Compound:Phospholipid:Phytosterol*:PEG- |
| | DMG |
| 40:5:53.5:1.5 | Compound:Phospholipid:Phytosterol*:PEG- |
| | DMG |
| 45:5:48.5:1.5 | Compound:Phospholipid:Phytosterol*:PEG- |
| | DMG |
| 50:5:43.5:1.5 | Compound:Phospholipid:Phytosterol*:PEG- |
| | DMG |
| 40:20:40:0 | Compound:Phospholipid:Phytosterol*:PEG- |
| | DMG |
| 45:20:35:0 | Compound:Phospholipid:Phytosterol*:PEG- |
| | DMG |
| 50:20:30:0 | Compound:Phospholipid:Phytosterol*:PEG- |
| | DMG |
| 55:20:25:0 | Compound:Phospholipid:Phytosterol*:PEG- |
| | DMG |
| 60:20:20:0 | Compound:Phospholipid:Phytosterol*:PEG- |
| | DMG |
| 40:15:45:0 | Compound:Phospholipid:Phytosterol*:PEG- |
| | DMG |

In some embodiments, transfer vehicle has a formulation as described in Table 11b.

For nanoparticle compositions including circRNA, solutions of the circRNA at concentrations of 0.1 mg/ml in deionized water are diluted in a buffer, e.g., 50 mM sodium citrate buffer at a pH between 3 and 4 to form a stock solution. Alternatively, solutions of the circRNA at concentrations of 0.15 mg/ml in deionized water are diluted in a buffer, e.g., 6.25 mM sodium acetate buffer at a pH between 3 and 4.5 to form a stock solution.

Nanoparticle compositions including a circular RNA and a lipid component are prepared by combining the lipid solution with a solution including the circular RNA at lipid component to circRNA wt:wt ratios between about 5:1 and about 50:1. The lipid solution is rapidly injected using, e.g., a NanoAssemblr microfluidic based system at flow rates between about 10 ml/min and about 18 ml/min or between about 5 ml/min and about 18 ml/min into the circRNA solution, to produce a suspension with a water to ethanol ratio between about 1:1 and about 4:1.

Nanoparticle compositions can be processed by dialysis to remove ethanol and achieve buffer exchange. Formulations are dialyzed twice against phosphate buffered saline (PBS), pH 7.4, at volumes 200 times that of the primary product using Slide-A-Lyzer cassettes (Thermo Fisher Scientific Inc., Rockford, IL) with a molecular weight cutoff of 10 kDa or 20 kDa. The formulations are then dialyzed overnight at 4° C. The resulting nanoparticle suspension is filtered through 0.2 pm sterile filters (Sarstedt, Numbrecht, Germany) into glass vials and sealed with crimp closures. Nanoparticle composition solutions of 0.01 mg/ml to 0.15 mg/ml are generally obtained.

The method described above induces nano-precipitation and particle formation.

Alternative processes including, but not limited to, T-junction and direct injection, may be used to achieve the same nano-precipitation. B. Characterization of nanoparticle compositions

A Zetasizer Nano ZS (Malvern Instruments Ltd, Malvern, Worcestershire, UK) can be used to determine the particle size, the polydispersity index (PDI) and the zeta potential of the nanoparticle compositions in 1×PBS in determining particle size and 15 mM PBS in determining zeta potential.

Ultraviolet-visible spectroscopy can be used to determine the concentration of circRNA in nanoparticle compositions. 100 μL of the diluted formulation in 1×PBS is added to 900 μL of a 4:1 (v/v) mixture of methanol and chloroform. After mixing, the absorbance spectrum of the solution is recorded, for example, between 230 nm and 330 nm on a DU 800 spectrophotometer (Beckman Coulter, Beckman Coulter, Inc., Brea, CA). The concentration of circRNA in the nanoparticle composition can be calculated based on the extinction coefficient of the circRNA used in the composition and on the difference between the absorbance at a wavelength of, for example, 260 nm and the baseline value at a wavelength of, for example, 330 nm.

A QUANT-IT™ RIBOGREEN® RNA assay (Invitrogen Corporation Carlsbad, CA) can be used to evaluate the encapsulation of circRNA by the nanoparticle composition. The samples are diluted to a concentration of approximately 5 μg/mL or 1 μg/mL in a TE buffer solution (10 mM Tris-HCl, 1 mM EDTA, pH 7.5). 50 μL of the diluted samples are transferred to a polystyrene 96 well plate and either 50 μL of TE buffer or 50 μL of a 2-4% Triton X-100 solution is added to the wells. The plate is incubated at a temperature of 37° C. for 15 minutes. The RIBOGREEN® reagent is diluted 1:100 or 1:200 in TE buffer, and 100 μL of this solution is added to each well. The fluorescence intensity can be measured using a fluorescence plate reader (Wallac Victor 1420 Multilabel Counter; Perkin Elmer, Waltham, MA) at an excitation wavelength of, for example, about 480 nm and an emission wavelength of, for example, about 520 nm. The fluorescence values of the reagent blank are subtracted from that of each of the samples and the percentage of free circRNA is determined by dividing the fluorescence intensity of the intact sample (without addition of Triton X-100) by the fluorescence value of the disrupted sample (caused by the addition of Triton X-100). C.

### Example 29B: In Vivo Formulation Studies

In order to monitor how effectively various nanoparticle compositions deliver circRNA to targeted cells, different nanoparticle compositions including circRNA are prepared and administered to rodent populations. Mice are intravenously, intramuscularly, intraarterially, or intratumorally administered a single dose including a nanoparticle composition with a lipid nanoparticle formulation. In some instances, mice may be made to inhale doses. Dose sizes may range from 0.001 mg/kg to 10 mg/kg, where 10 mg/kg describes a dose including 10 mg of a circRNA in a nanoparticle composition for each 1 kg of body mass of the mouse. A control composition including PBS may also be employed.

Upon administration of nanoparticle compositions to mice, dose delivery profiles, dose responses, and toxicity of particular formulations and doses thereof can be measured by enzyme-linked immunosorbent assays (ELISA), bioluminescent imaging, or other methods. Time courses of protein expression can also be evaluated. Samples collected from the rodents for evaluation may include blood and tissue (for example, muscle tissue from the site of an intramuscular injection and internal tissue); sample collection may involve sacrifice of the animals.

Higher levels of protein expression induced by administration of a composition including a circRNA will be indicative of higher circRNA translation and/or nanoparticle composition circRNA delivery efficiencies. As the non-RNA components are not thought to affect translational machineries themselves, a higher level of protein expression is likely indicative of a higher efficiency of delivery of the circRNA by a given nanoparticle composition relative to other nanoparticle compositions or the absence thereof.

### Example 30

### Characterization of Nanoparticle Compositions

A Zetasizer Nano ZS (Malvern Instruments Ltd, Malvern, Worcestershire, UK) can be used to determine the particle size, the polydispersity index (PDI) and the zeta potential of the transfer vehicle compositions in 1×PBS in determining particle size and 15 mM PBS in determining zeta potential.

Ultraviolet-visible spectroscopy can be used to determine the concentration of a therapeutic and/or prophylactic (e.g., RNA) in transfer vehicle compositions. 100 μL of the diluted formulation in 1×PBS is added to 900 μL of a 4:1 (v/v) mixture of methanol and chloroform. After mixing, the absorbance spectrum of the solution is recorded, for example, between 230 nm and 330 nm on a DU 800 spectrophotometer (Beckman Coulter, Beckman Coulter, Inc., Brea, CA). The concentration of therapeutic and/or prophylactic in the transfer vehicle composition can be calculated based on the extinction coefficient of the therapeutic and/or prophylactic used in the composition and on the difference between the absorbance at a wavelength of, for example, 260 nm and the baseline value at a wavelength of, for example, 330 nm.

For transfer vehicle compositions including RNA, a QUANT-IT™ RIBOGREEN® RNA assay (Invitrogen Corporation Carlsbad, CA) can be used to evaluate the encapsulation of RNA by the transfer vehicle composition. The samples are diluted to a concentration of approximately 5 μg/mL or 1 μg/mL in a TE buffer solution (10 mM Tris-HCl, 1 mM EDTA, pH 7.5). 50 μL of the diluted samples are transferred to a polystyrene 96 well plate and either 50 μL of TE buffer or 50 μL of a 2-4% Triton X-100 solution is added to the wells. The plate is incubated at a temperature of 37° C. for 15 minutes. The RIBOGREEN® reagent is diluted 1:100 or 1:200 in TE buffer, and 100 μL of this solution is added to each well. The fluorescence intensity can be measured using a fluorescence plate reader (Wallac Victor 1420 Multilablel Counter; Perkin Elmer, Waltham, MA) at an excitation wavelength of, for example, about 480 nm and an emission wavelength of, for example, about 520 nm. The fluorescence values of the reagent blank are subtracted from that of each of the samples and the percentage of free RNA is determined by dividing the fluorescence intensity of the intact sample (without addition of Triton X-100) by the fluorescence value of the disrupted sample (caused by the addition of Triton X-100).

### Example 31

### T Cell Targeting

To target transfer vehicles to T-cells, T cell antigen binders, e.g., anti-CD8 antibodies, are coupled to the surface of the transfer vehicle. Anti-T cell antigen antibodies are mildly reduced with an excess of DTT in the presence of EDTA in PBS to expose free hinge region thiols. To remove DTT, antibodies are passed through a desalting column. The heterobifunctional cross-linker SM(PEG)24 is used to anchor antibodies to the surface of circRNA-loaded transfer vehicles (Amine groups are present in the head groups of PEG lipids, free thiol groups on antibodies were created by DTT, SM(PEG)24 cross-links between amines and thiol groups). Transfer vehicles are first incubated with an excess of SM(PEG)24 and centrifuged to remove unreacted cross-linker. Activated transfer vehicles are then incubated with an excess of reduced anti-T cell antigen antibody. Unbound antibody is removed using a centrifugal filtration device.

### Example 32

### RNA Containing Transfer Vehicle Using RV88.

In this example RNA containing transfer vehicles are synthesized using the 2-D vortex microfluidic chip with the cationic lipid RV88 for delivery of circRNA.

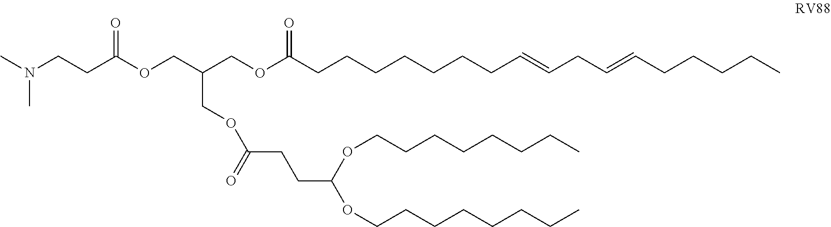 [SMILES: CCCCC/C=C/C/C=C/CCCCCCCC(=O)OCC(COC(=O)CCC(OCCCCCCCC)OCCCCCCCC)COC(=O)CCN(C)C]

TABLE 12a

| Materials and Instrument | Vendor | Cat # |
|---|---|---|
| 1M Tris-HCl, pH 8.0, Sterile | Teknova | T1080 |
| 5M Sodium Chloride solution | Teknova | S0250 |
| QB Citrate buffer, pH 6.0 | Teknova | Q2446 |
| (100 mM) | | |
| Nuclease-free water | Ambion | AM9937 |
| Triton X-100 | Sigma-Aldrich | T8787-100ML |
| RV88 | GVK bio | |
| DSPC | Lipoid | 556500 |
| Cholesterol | Sigma | C3045-5G |
| PEG2K | Avanti Polar Lipids | 880150 |
| Ethanol | Acros Organic | 615090010 |
| 5 mL Borosilicate glass vials | Thermo Scientific | ST5-20 |
| PD MiniTrap G-25 Desalting | GE Healthcare | VWR Cat |
| Columns | | #95055-984 |
| Quant-iT RiboGreen RNA | Molecular Probes/ | R11490 |
| Assay kit | Life Technologies | |
| Black 96-well microplates | Greiner | 655900 |

RV88, DSPC, and cholesterol all being prepared in ethanol at a concentration of 10 mg/ml in borosilica vials. The lipid 14:0-PEG2K PE is prepared at a concentration of 4 mg/ml also in a borosilica glass vial. Dissolution of lipids at stock concentrations is attained by sonication of the lipids in ethanol for 2 min. The solutions are then heated on an orbital tilting shaker set at 170 rpm at 37° C. for 10 min. Vials are then equilibrated at 26° C. for a minimum of 45 min. The lipids are then mixed by adding volumes of stock lipid as shown in Table 12b. The solution is then adjusted with ethanol such that the final lipid concentration was 7.92 mg/ml.

TABLE 12b

| | | | | | Stock | | Eth- |
|---|---|---|---|---|---|---|---|
| | | | | | (mg/ | | anol |
| Composition | MW | % | nmoles | mq | ml) | ul | (ul) |
| RV88 | 794.2 | 40% | 7200 | 5.72 | 10 | 571.8 | 155.3 |
| DSPC | 790.15 | 10% | 1800 | 1.42 | 10 | 142.2 | |
| Cholesterol | 386.67 | 48% | 8640 | 3.34 | 10 | 334.1 | |
| PEG2K | 2693.3 | 2% | 360 | 0.97 | 4 | 242.4 | |

RNA is prepared as a stock solution with 75 mM Citrate buffer at pH 6.0 and a concentration of RNA at 1.250 mg/ml. The concentration of the RNA is then adjusted to 0.1037 mg/ml with 75 mM citrate buffer at pH 6.0, equilibrated to 26° C. The solution is then incubated at 26° C. for a minimum of 25 min.

The microfluidic chamber is cleaned with ethanol and neMYSIS syringe pumps are prepared by loading a syringe with the RNA solution and another syringe with the ethanolic lipid. Both syringes are loaded and under the control of neMESYS software. The solutions are then applied to the mixing chip at an aqueous to organic phase ratio of 2 and a total flow rate of 22 ml/min (14.67 ml/min for RNA and 7.33 ml/min for the lipid solution. Both pumps are started synchronously. The mixer solution that flowed from the microfluidic chip is collected in 4×1 ml fractions with the first fraction being discarded as waste. The remaining solution containing the RNA-liposomes is exchanged by using G-25 mini desalting columns to 10 mM Tris-HCI, 1 mM EDTA, at pH 7.5. Following buffer exchange, the materials are characterized for size, and RNA entrapment through DLS analysis and Ribogreen assays, respectively.

### Example 33

### RVA Containing Transfer Vehicle Using RV94.

In this example, RNA containing liposome are synthesized using the 2-D vortex microfluidic chi with the cationic lipid RV94 for delivery of circRNA.

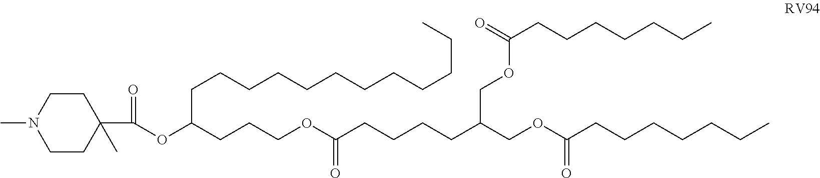 [SMILES: CCCCCCCCCCCCC(CCCOC(=O)CCCCC(COC(=O)CCCCCCC)COC(=O)CCCCCCC)OC(=O)C1(C)CCN(C)CC1]

TABLE 13

| Materials and Instrument | Vendor | Cat # |
|---|---|---|
| 1M Tris-HCl, pH 8.0, Sterile | Teknova | T1080 |
| 5M Sodium Chloride solution | Teknova | S0250 |
| QB Citrate buffer, pH 6.0 | Teknova | Q2446 |
| (100 mM) | | |
| Nuclease-free water | Ambion | AM9937 |
| Triton X-100 | Sigma-Aldrich | T8787- |
| | | 100ML |
| RV94 | GVKbio | |
| DSPC | Lipoid | 556500 |
| Cholesterol | Sigma | C3045-5G |
| PEG2K | Avanti Polar Lipids | 880150 |
| Ethanol | Acros Organic | 615090010 |
| 5 ml Borosilicate glass vials | Thermo Scientific | ST5-20 |
| PD MiniTrap G-25 Desalting | GE Healthcare | VWR Cat. |
| Columns | | #95055-984 |
| Quant-iT RiboGreen | Molecular Probes/Life | R11490 |
| RNA Assay kit | Technologies | |
| Black 96-well microplates | Greiner | 655900 |

The lipids were prepared as in Example 29 using the material amounts named in Table 14 to a final lipid concentration of 7.92 mg/ml.

TABLE 14

| | | | | | Stock | | Eth- |
|---|---|---|---|---|---|---|---|
| | | | | | (mg/ | | anol |
| Composition | MW | % | nmoles | mq | ml) | ul | (ul) |
| RV94 | 808.22 | 40% | 2880 | 2.33 | 10 | 232.8 | 155.3 |
| DSPC | 790.15 | 10% | 720 | 0.57 | 10 | 56.9 | |
| Cholesterol | 386.67 | 48% | 3456 | 1.34 | 10 | 133.6 | |
| PEG2K | 2693.3 | 2% | 144 | 0.39 | 4 | 97.0 | |

The aqueous solution of circRNA is prepared as a stock solution with 75 mM Citrate buffer at pH 6.0 the circRNA at 1.250 mg/ml. The concentration of the RNA is then adjusted to 0.1037 mg/ml with 75 mM citrate buffer at pH 6.0, equilibrated to 26° C. The solution is then incubated at 26° C. for a minimum of 25 min.

The microfluidic chamber is cleaned with ethanol and neMYSIS syringe pumps are prepared by loading a syringe with the RNA solution and another syringe with the ethanolic lipid. Both syringes are loaded and under the control of neMESYS software. The solutions are then applied to the mixing chip at an aqueous to organic phase ratio of 2 and a total flow rate of 22 ml/min (14.67 ml/min for RNA and 7.33 ml/min for the lipid solution. Both pumps are started synchronously. The mixer solution that flowed from the microfluidic chip is collected in 4×1 ml fractions with the first fraction being discarded as waste. The remaining solution containing the circRNA-transfer vehicles is exchanged by using G-25 mini desalting columns to 10 mM Tris-HCl, 1 mM EDTA, at pH 7.5, as described above. Following buffer exchange, the materials are characterized for size, and RNA entrapment through DLS analysis and Ribogreen assays, respectively. The biophysical analysis of the liposomes is shown in Table 15.

TABLE 15

| | | | | RNA | RNA | | |
|---|---|---|---|---|---|---|---|
| | | | | encap- | encap- | | |
| | | TFR | Ratio | sulation | sulation | | |
| Sample | N:P | ml/ | (aqueous/ | amount | yield | size | |
| Name | Ratio | min | org phase) | (μg/ml) | % | d · nm | PDI |
| SAM- | 8 | 22 | 2 | 31.46 | 86.9 | 113.1 | 0.12 |
| RV94 | | | | | | | |

### Example 34

### General Protocol for in Line Mixing.

Individual and separate stock solutions are prepared—one containing lipid and the other circRNA. Lipid stock containing a desired lipid or lipid mixture, DSPC, cholesterol and PEG lipid is prepared by solubilized in 90% ethanol. The remaining 10% is low pH citrate buffer. The concentration of the lipid stock is 4 mg/mL. The pH of this citrate buffer can range between pH 3 and pH 5, depending on the type of lipid employed. The circRNA is also solubilized in citrate buffer at a concentration of 4 mg/mL. 5 mL of each stock solution is prepared.

Stock solutions are completely clear and lipids are ensured to be completely solubilized before combining with circRNA. Stock solutions may be heated to completely solubilize the lipids. The circRNAs used in the process may be unmodified or modified oligonucleotides and may be conjugated with lipophilic moieties such as cholesterol.

The individual stocks are combined by pumping each solution to a T-junction. A dual-head Watson-Marlow pump was used to simultaneously control the start and stop of the two streams. A 1.6 mm polypropylene tubing is further downsized to 0.8 mm tubing in order to increase the linear flow rate. The polypropylene line (ID=0.8 mm) are attached to either side of a T-junction. The polypropylene T has a linear edge of 1.6 mm for a resultant volume of 4.1 mm³. Each of the large ends (1.6 mm) of polypropylene line is placed into test tubes containing either solubilized lipid stock or solubilized circRNA. After the T-junction, a single tubing is placed where the combined stream exited. The tubing is then extended into a container with 2× volume of PBS, which is rapidly stirred. The flow rate for the pump is at a setting of 300 rpm or 110 mL/min. Ethanol is removed and exchanged for PBS by dialysis. The lipid formulations are then concentrated using centrifugation or diafiltration to an appropriate working concentration.

C57BL/6 mice (Charles River Labs, MA) receive either saline or formulated circRNA via tail vein injection. At various time points after administration, serum samples are collected by retroorbital bleed. Serum levels of Factor VII protein are determined in samples using a chromogenic assay (Biophen FVTI, Aniara Corporation, OH). To determine liver RNA levels of Factor VII, animals are sacrificed and livers are harvested and snap frozen in liquid nitrogen. Tissue lysates are prepared from the frozen tissues and liver RNA levels of Factor VII are quantified using a branched DNA assay (QuantiGene Assay, Panomics, CA).

FVII activity is evaluated in FVTI siRNA-treated animals at 48 hours after intravenous (bolus) injection in C57BL/6 mice. FVII is measured using a commercially available kit for determining protein levels in serum or tissue, following the manufacturer's instructions at a microplate scale. FVII reduction is determined against untreated control mice, and the results are expressed as % Residual FVII. Two dose levels (0.05 and 0.005 mg/kg FVII siRNA) are used in the screen of each novel liposome composition.

### Example 36

circRNA Formulation Using Preformed Vesicles.

Cationic lipid containing transfer vehicles are made using the preformed vesicle method. Cationic lipid, DSPC, cholesterol and PEG-lipid are solubilized in ethanol at a molar ratio of 40/10/40/10, respectively. The lipid mixture is added to an aqueous buffer (50 mM citrate, pH 4) with mixing to a final ethanol and lipid concentration of 30% (vol/vol) and 6.1 mg/mL respectively and allowed to equilibrate at room temperature for 2 min before extrusion. The hydrated lipids are extruded through two stacked 80 nm pore-sized filters (Nuclepore) at 22° C. using a Lipex Extruder (Northern Lipids, Vancouver, BC) until a vesicle diameter of 70-90 nm, as determined by Nicomp analysis, is obtained. For cationic lipid mixtures which do not form small vesicles, hydrating the lipid mixture with a lower pH buffer (50 mM citrate, pH 3) to protonate the phosphate group on the DSPC headgroup helps form stable 70-90 nm vesicles.

The FVII circRNA (solubilised in a 50 mM citrate, pH 4 aqueous solution containing 30% ethanol) is added to the vesicles, pre-equilibrated to 35° C., at a rate of −5 mL/min with mixing. After a final target circRNA/lipid ratio of 0.06 (wt wt) is achieved, the mixture is incubated for a further 30 min at 35° C. to allow vesicle re-organization and encapsulation of the FVII RNA. The ethanol is then removed and the external buffer replaced with PBS (155 mM NaCl, 3 mM Na2HPO4, 1 mM KH2PO4, pH 7.5) by either dialysis or tangential flow diafiltration. The final encapsulated circRNA-to-lipid ratio is determined after removal of unencapsulated RNA using size-exclusion spin columns or ion exchange spin columns.

### Example 37

### Example 37A: Expression of Trispecific Antigen Binding Proteins from Engineered Circular RNA

Circular RNAs are designed to include. (1) a 3′ post splicing group I intron fragment; (2) an Internal Ribosome Entry Site (IRES); (3) a trispecific antigen-binding protein coding region; and (4) a 3′ homology region. The trispecific antigen-binding protein regions are constructed to produce an exemplary trispecific antigen-binding protein that will bind to a target antigen, e.g., GPC3.

### Example 37B: Generation of a scFv CD3 Binding Domain

The human CD3epsilon chain canonical sequence is Uniprot Accession No. P07766. The human CD3gamma chain canonical sequence is Uniprot Accession No. P09693. The human CD3delta chain canonical sequence is Uniprot Accession No. P043234. Antibodies against CD3epsilon, CD3gamma or CD3delta are generated via known technologies such as affinity maturation. Where murine anti-CD3 antibodies are used as a starting material, humanization of murine anti-CD3 antibodies is desired for the clinical setting, where the mouse-specific residues may induce a human-anti-mouse antigen (HAMA) response in subjects who receive treatment of a trispecific antigen-binding protein described herein. Humanization is accomplished by grafting CDR regions from murine anti-CD3 antibody onto appropriate human germline acceptor frameworks, optionally including other modifications to CDR and/or framework regions.

Human or humanized anti-CD3 antibodies are therefore used to generate scFv sequences for CD3 binding domains of a trispecific antigen-binding protein. DNA sequences coding for human or humanized VL and VH domains are obtained, and the codons for the constructs are, optionally, optimized for expression in cells from Homo sapiens. The order in which the VL and VH domains appear in the scFv is varied (i.e. VL-VH, or VH-VL orientation), and three copies of the “G4S” or “G₄S” subunit (G₄S)₃ connect the variable domains to create the scFv domain. Anti-CD3 scFv plasmid constructs can have optional Flag, His or other affinity tags, and are electroporated into HEK293 or other suitable human or mammalian cell lines and purified. Validation assays include binding analysis by FACS, kinetic analysis using Proteon, and staining of CD3-expressing cells.

### Example 37C: Generation of a scFv Glypican-3 (GPC3) Binding Domain

Glypican-3 (GPC3) is one of the cell surface proteins present on Hepatocellular Carcinoma but not on healthy normal liver tissue. It is frequently observed to be elevated in hepatocellular carcinoma and is associated with poor prognosis for HCC patients. It is known to activate Wnt signalling. GPC3 antibodies have been generated including MDX-1414, HN3, GC33, and YP7.

A scFv binding to GPC-3 or another target antigen is generated similarly to the above method for generation of a scFv binding domain to CD3.

### Example 37D: Expression of Trispecific Antigen-Binding Proteins In Vitro

A CHO cell expression system (Flp-In®, Life Technologies), a derivative of CHO-K1 Chinese Hamster ovary cells (ATCC, CCL-61) (Kao and Puck, Proc. Natl. Acad Sci USA 1968; 60(4):1275-81), is used. Adherent cells are subcultured according to standard cell culture protocols provided by Life Technologies.

For adaption to growth in suspension, cells are detached from tissue culture flasks and placed in serum-free medium. Suspension-adapted cells are cryopreserved in medium with 10% DMSO.

Recombinant CHO cell lines stably expressing secreted trispecific antigen-binding proteins are generated by transfection of suspension-adapted cells. During selection with the antibiotic Hygromycin B viable cell densities are measured twice a week, and cells are centrifuged and resuspended in fresh selection medium at a maximal density of 0.1×10⁶ viable cells/mL. Cell pools stably expressing trispecific antigen-binding proteins are recovered after 2-3 weeks of selection at which point cells are transferred to standard culture medium in shake flasks. Expression of recombinant secreted proteins is confirmed by performing protein gel electrophoresis or flow cytometry. Stable cell pools are cryopreserved in DMSO containing medium.

Trispecific antigen-binding proteins are produced in 10-day fed-batch cultures of stably transfected CHO cell lines by secretion into the cell culture supernatant. Cell culture supernatants are harvested after 10 days at culture viabilities of typically >75%. Samples are collected from the production cultures every other day and cell density and viability are assessed. On day of harvest, cell culture supernatants are cleared by centrifugation and vacuum filtration before further use.

Protein expression titers and product integrity in cell culture supernatants are analyzed by SDS-PAGE.

### Example 37E: Purification of Trispecific Antigen-Binding Proteins

Trispecific antigen-binding proteins are purified from CHO cell culture supernatants in a two-step procedure. The constructs are subjected to affinity chromatography in a first step followed by preparative size exclusion chromatography (SEC) on Superdex 200 in a second step. Samples are buffer-exchanged and concentrated by ultrafiltration to a typical concentration of >1 mg/mL Purity and homogeneity (typically >90%) of final samples are assessed by SDS PAGE under reducing and non-reducing conditions, followed by immunoblotting using an anti-(half-life extension domain) or anti idiotype antibody as well as by analytical SEC, respectively. Purified proteins are stored at aliquots at −80° C. until use.

### Example 38

Expression of Engineered Circular RNA with a Half-Life Extension Domain has Improved Pharmacokinetic Parameters than without a Half-Life Extension Domain

The trispecific antigen-binding protein encoded on a circRNA molecule of example 23 is administered to cynomolgus monkeys as a 0.5 mg/kg bolus injection intramuscularly. Another cynomolgus monkey group receives a comparable protein encoded on a circRNA molecule in size with binding domains to CD3 and GPC-3, but lacking a half-life extension domain. A third and fourth group receive a protein encoded on a circRNA molecule with CD3 and half-life extension domain binding domains and a protein with GPC-3 and half-life extension domains, respectively. Both proteins encoded by circRNA are comparable in size to the trispecific antigen-binding protein. Each test group consists of 5 monkeys. Serum samples are taken at indicated time points, serially diluted, and the concentration of the proteins is determined using a binding ELISA to CD3 and/or GPC-3.

Pharmacokinetic analysis is performed using the test article plasma concentrations. Group mean plasma data for each test article conforms to a multi-exponential profile when plotted against the time post-dosing. The data are fit by a standard two-compartment model with bolus input and first-order rate constants for distribution and elimination phases. The general equation for the best fit of the data for i.v. administration is: c(t)=Ae^{-at}+Be^{-pt}, where c(t) is the plasma concentration at time t, A and B are intercepts on the Y-axis, and a and β are the apparent first-order rate constants for the distribution and elimination phases, respectively. The a-phase is the initial phase of the clearance and reflects distribution of the protein into all extracellular fluid of the animal, whereas the second or β-phase portion of the decay curve represents true plasma clearance. Methods for fitting such equations are well known in the art. For example, A=D/V(a−k21)/(a−p), B=D/V(p−k21)/(a−p), and a and β (for α>β) are roots of the quadratic equation: r²+(k12+k21+k10)r+k21k10=0 using estimated parameters of V=volume of distribution, k10=elimination rate, k12=transfer rate from compartment 1 to compartment 2 and k21=transfer rate from compartment 2 to compartment 1, and D=the administered dose.

Data analysis: Graphs of concentration versus time profiles are made using KaleidaGraph (KaleidaGraph™ V. 3.09 Copyright 1986-1997. Synergy Software. Reading, Pa.). Values reported as less than reportable (LTR) are not included in the PK analysis and are not represented graphically. Pharmacokinetic parameters are determined by compartmental analysis using WinNonlin software (WinNonlin) Professional V. 3.1 WinNonlin™ Copyright 1998-1999. Pharsight Corporation. Mountain View, Calif). Pharmacokinetic parameters are computed as described in Ritschel W A and Kearns G L, 1999, EST: Handbook Of Basic Pharmacokinetics Including Clinical Applications, 5th edition, American Pharmaceutical Assoc., Washington, D C.

It is expected that the trispecific antigen-binding protein encoded on a circRNA molecule of Example 23 has improved pharmacokinetic parameters such as an increase in elimination half-time as compared to proteins lacking a half-life extension domain.

### Example 39

### Cytotoxicity of the Trispecific Antigen-Binding Protein

The trispecific antigen-binding protein encoded on a circRNA molecule of Example 23 is evaluated in vitro on its mediation of T cell dependent cytotoxicity to GPC-3+ target cells.

Fluorescence labeled GPC3 target cells are incubated with isolated PBMC of random donors or T-cells as effector cells in the presence of the trispecific antigen-binding protein of Example 23. After incubation for 4 h at 37° C. in a humidified incubator, the release of the fluorescent dye from the target cells into the supernatant is determined in a spectrofluorimeter. Target cells incubated without the trispecific antigen-binding protein of Example 23 and target cells totally lysed by the addition of saponin at the end of the incubation serve as negative and positive controls, respectively.

Based on the measured remaining living target cells, the percentage of specific cell lysis is calculated according to the following formula: [1−(number of living targets(sample)/number of living targets(spontaneous))]×100%. Sigmoidal dose response curves and EC50 values are calculated by non-linear regression/4-parameter logistic fit using the GraphPad Software. The lysis values obtained for a given antibody concentration are used to calculate sigmoidal dose-response curves by 4 parameter logistic fit analysis using the Prism software.

### Example 40

### Synthesis of Ionizable Lipids

### 40.1 Synthesis of ((3-(2-methyl-1H-imidazol-1-yl)propyl)azanediyl)bis(hexane-6,1-diyl) bis(2-hexyldecanoate)(Lipid 10a-27) and ((3-(1H-imidazol-1-yl)propyl)azanediyl)bis(hexane-6,1-diyl) bis(2-hexyldecanoate))(Lipid 10a-26)

In a 100 mL round bottom flask connected with condenser, 3-(1H-imidazol-1-yl)propan-1-amine (100 mg, 0.799 mmol) or 3-(2-methyl-1H-imidazol-1-yl)propan-1-amine (0.799 mmol), 6-bromohexyl 2-hexyldecanoate (737.2 mg, 1.757 mmol), potassium carbonate (485 mg, 3.515 mmol) and potassium iodide (13 mg, 0.08 mmol) were mixed in acetonitrile (30 mL), and the reaction mixture was heated to 80° C. for 48 h. The mixture was cooled to room temperature and was filtered through a pad of Celite. The filtrate was diluted with ethyl acetate. After washing with water, brine and dried over anhydrous sodium sulfate. The solvent was evaporated and the crude residue was purified by flash chromatography (SiO₂:CH₂Cl₂=100% to 10% of methanol in CH2C12) and colorless oil product was obtained (92 mg, 15%). Molecular formula of ((3-(JH-imidazol-1-yl)propyl)azanediyl)bis(hexane-6,1-diyl) bis(2-hexyldecanoate)) is Ca₅₀H₉₅N₃O₄ and molecular weight (Mw) is 801.7.

### Reaction scheme for synthesis of ((3-(1H-imidazo/1-yl)propyl)azanediyl)bis(hexane-6,1-diyl) bis(2-hexyldecanoate)) (Lipid 10a-26)

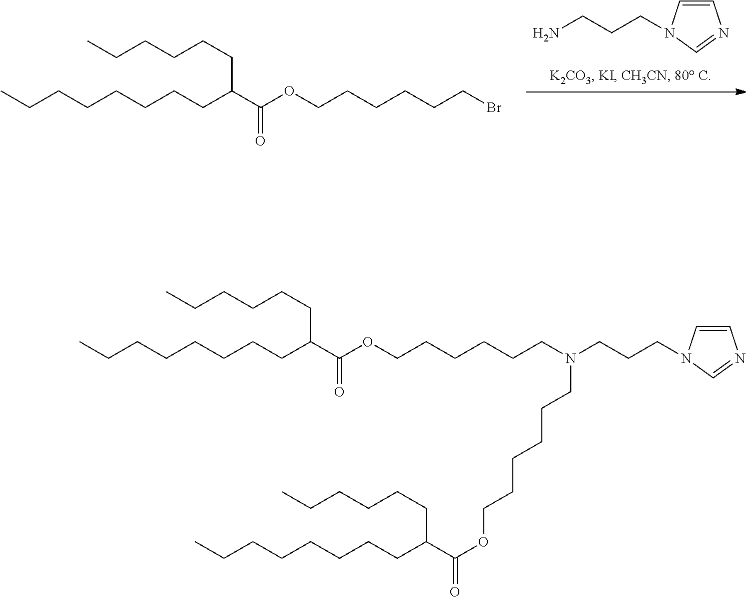 [SMILES: CCCCCCCCC(CCCCCC)C(=O)OCCCCCCBr ; NCCCn1ccnc1 ; CCCCCCCCC(CCCCCC)C(=O)OCCCCCCN(CCCCCCOC(=O)C(CCCCCC)CCCCCCCC)CCCn1ccnc1]

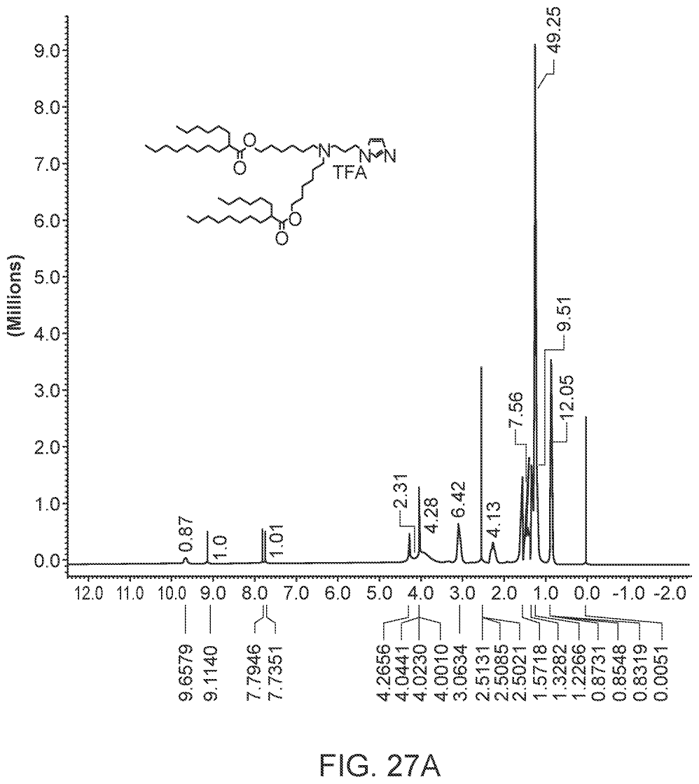

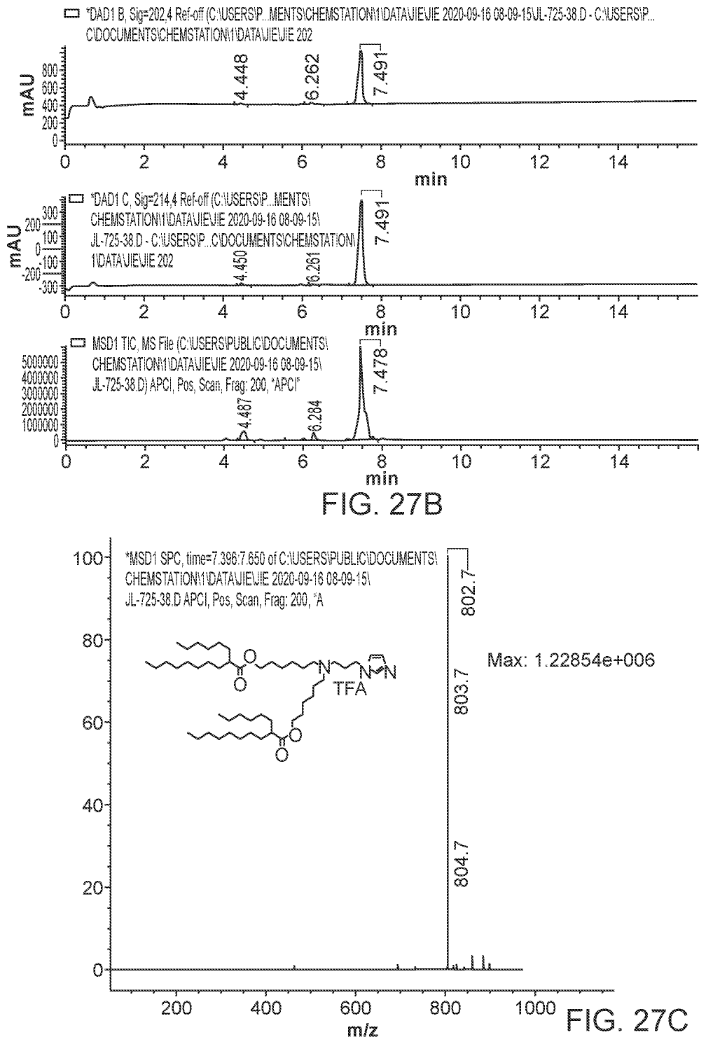

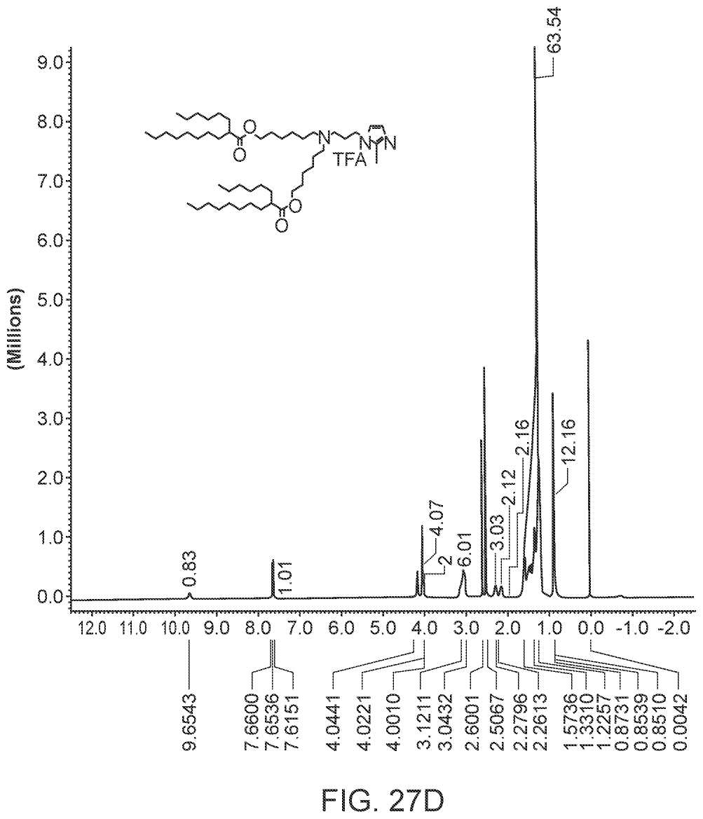

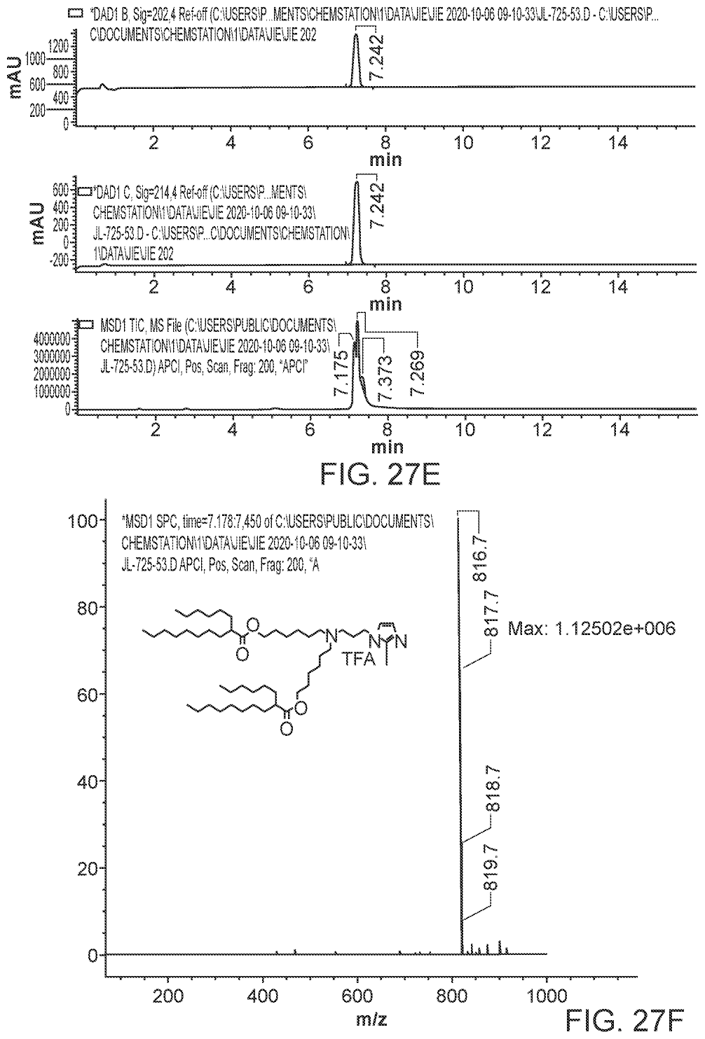

Characterization of Lipid 10a-26 was performed by LC-MS. FIG. 27A-C shows characterization of Lipid 10a-26. FIG. 27A shows the proton NMR observed for Lipid 10a-26. FIG. 27B is a representative LC/MS trace for Lipid 10a-26 with total ion and UV chromatograms shown.

### 40.2 Synthesis of Lipid 22-S14

### 40.2.1 Synthesis of 2-(tetradecylthio)ethan-1-ol

To a mixture of 2-sulfanylethanol (5.40 g, 69.11 mmol, 4.82 mL, 0.871 eq) in acetonitrile (200 mL) was added I-Bromotetradecane (22 g, 79.34 mmol, 23.66 mL, 1 eq) and potassium carbonate (17.55 g, 126.95 mmol, 1.6 eq) at 25° C. The reaction mixture was warmed to 40° C. and stirred for 12 hr. TLC (ethyl acetate/petroleum ether=25/1, R_{f}=0.3, stained by 12) showed the starting material was consumed completely and a new main spot was generated. The reaction mixture was filtered and the filter cake was washed with acetonitrile (50 mL) and then the filtrate was concentrated under vacuum to get a residue which was purified by column on silica gel (ethyl acetate/petroleum ether=1/100 to 1/25) to afford 2-(tetradecylthio)ethan-1-ol (14 g, yield 64.28%) as a white solid.

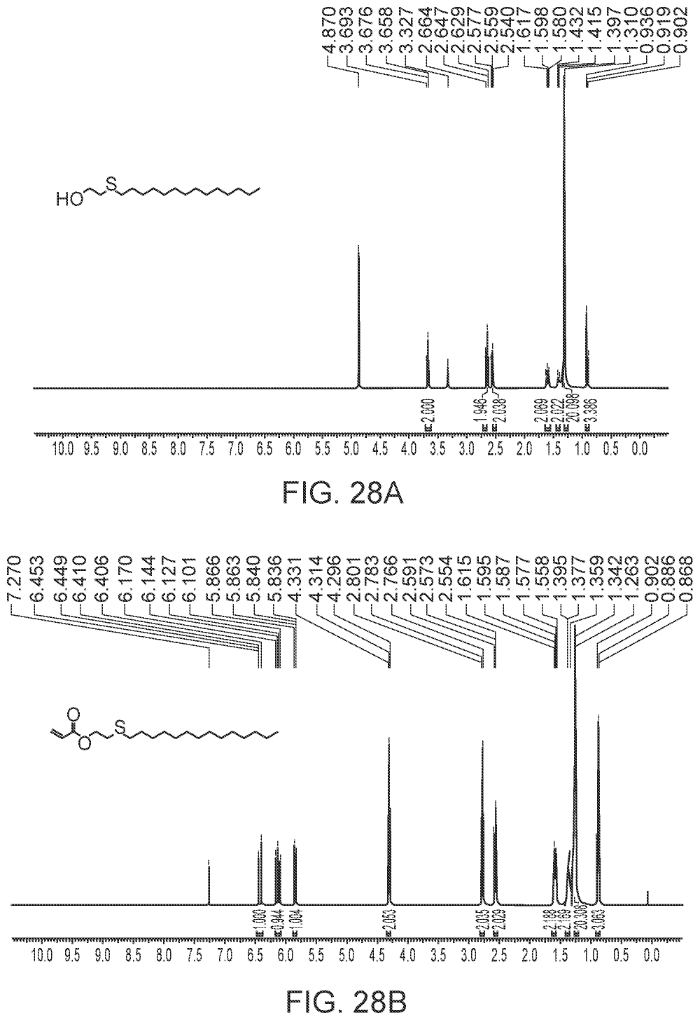

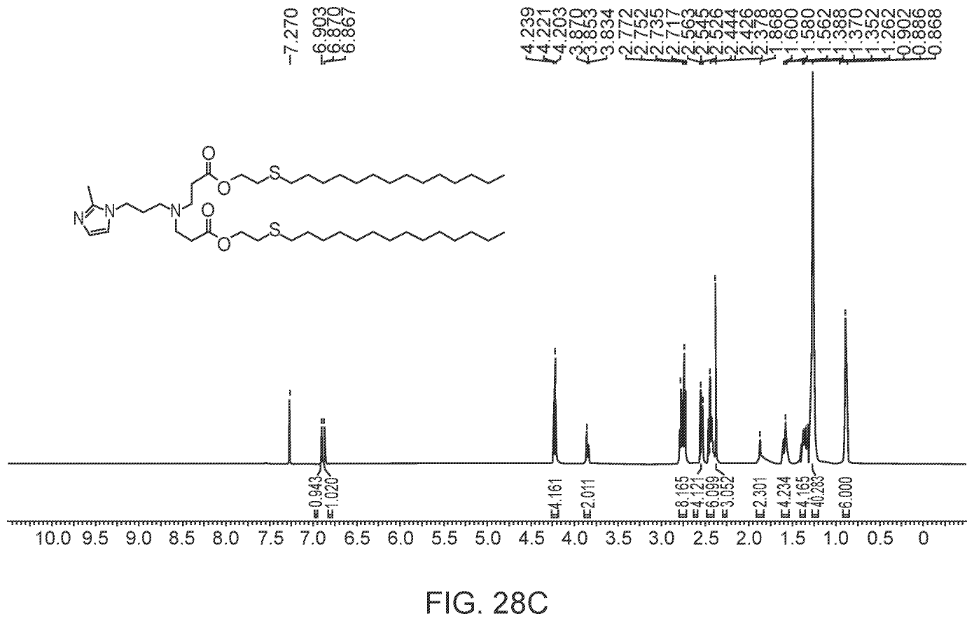

¹H NMR (ET36387-45-P1A, 400 MHz, CHLOROFORM-d) δ 0.87-0.91 (m, 3H) 1.27 (s, 20H) 1.35-1.43 (m, 2H) 1.53-1.64 (m, 2H) 2.16 (br s, 1H) 2.49-2.56 (m, 2H) 2.74 (t, J=5.93 Hz, 2H) 3.72 (br d, J=4.89 Hz, 2H). FIG. 28 shows corresponding Nuclear Magnetic Resonance (NMR) spectrum.

### 40.2.2 Synthesis of 2-(tetradecylthio)ethyl acrylate

To a solution of 2-(tetradecylthio)ethan-1-ol (14 g, 51.00 mmol, 1 eq) in dichloromethane (240 mL) was added triethylamine (7.74 g, 76.50 mmol, 10.65 mL, 1.5 eq) and prop-2-enoyl chloride (5.54 g, 61.20 mmol, 4.99 mL, 1.2 eq) dropwise at 0° C. under nitrogen. The reaction mixture was warmed to 25° C. and stirred for 12 hr. TLC (ethyl acetate/petroleum ether=25/1, Rf=0.5, stained by 12) showed the starting material was consumed completely and a new main spot was generated. The reaction solution was concentrated under vacuum to get crude which was purified by column on silica gel (ethyl acetate/petroleum ether=1/100 to 1/25) to afford 2-(tetradecylthio)ethyl acrylate (12 g, yield 71.61%) as a colorless oil.

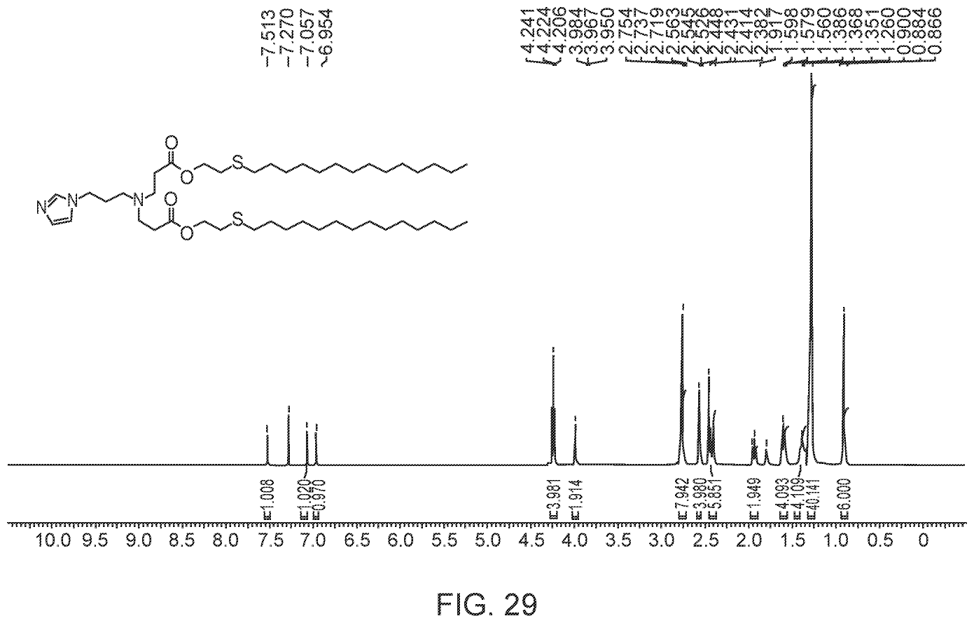

¹H NMR (ET36387-49-PIA, 400 MHz, CHLOROFORM-d) δ 0.85-0.93 (m, 3H) 1.26 (s, 19H) 1.35-1.43 (m, 2H) 1.53-1.65 (m, 2H) 2.53-2.62 (m, 2H) 2.79 (t, J=7.03 Hz, 2H) 4.32 (t, 0.1=7.03 Hz, 2H) 5.86 (dd, J=10.39, 1.47 Hz, 1H) 6.09-6.19 (m, 1H) 6.43 (dd, J=17.30, 1.41 Hz, 1H). FIG. 29 shows corresponding Nuclear Magnetic Resonance (NMR) spectrum.

### 40.2.3 Synthesis of bis(2-(tetradecylthio)ethyl) 3,3′-((3-(2-methyl-1H-imidazol-1-yl)propyl)azanediyl)dipropionate (Lipid 22-S4)

A flask was charged with 3-(2-methyl-1H-imidazol-1-yl)propan-1-amine (300 mg, 2.16 mmol) and 2-(tetradecylthio)ethyl acrylate (1.70 g, 5.17 mmol). The neat reaction mixture was heated to 80° C. and stirred for 48 hr. TLC (ethyl acetate, R_{f}=0.3, stained by 12, one drop ammonium hydroxide added) showed the starting material was consumed completely and a new main spot was formed. The reaction mixture was diluted with dichloromethane (4 mL) and purified by column on silica gel (petroleum ether/ethyl acetate=3/1 to 0/1, 0.1% ammonium hydroxide added) to get bis(2-(tetradecylthio)ethyl) 3,3′-((3-(2-methyl-1H-imidazol-1-yl)propyl)azanediyl)dipropionate (501 mg, yield 29.10%) as colorless oil.

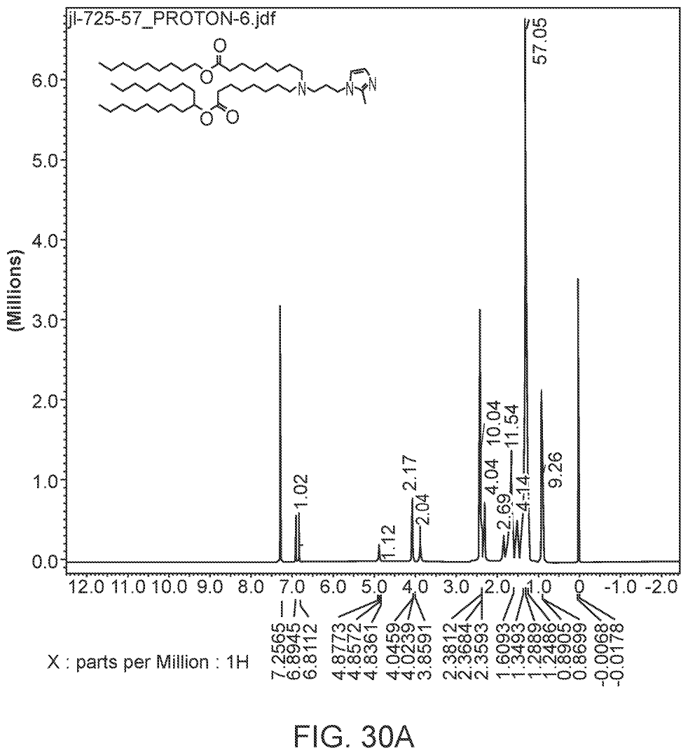

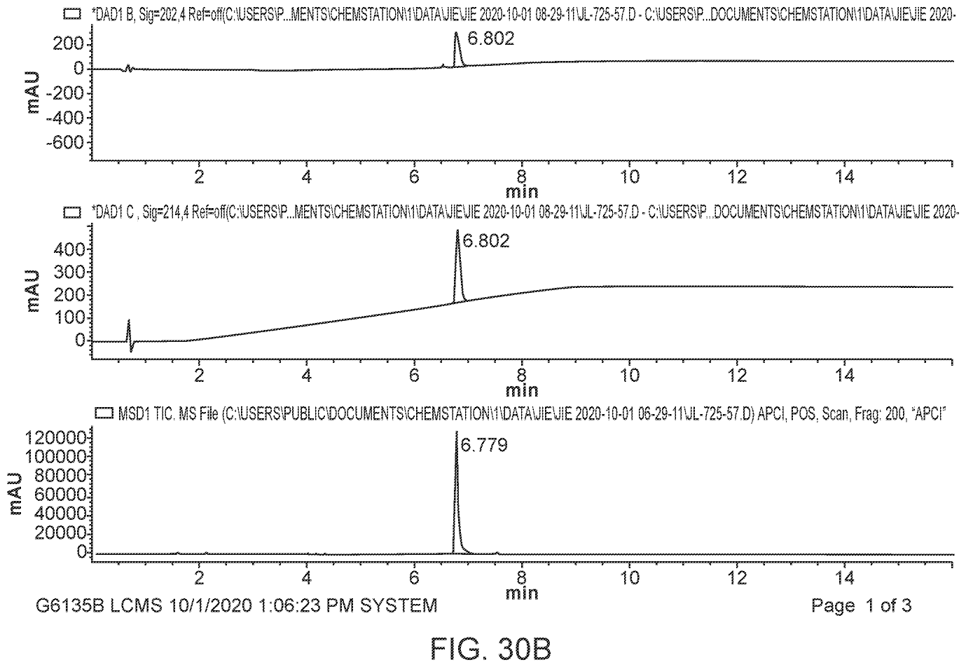

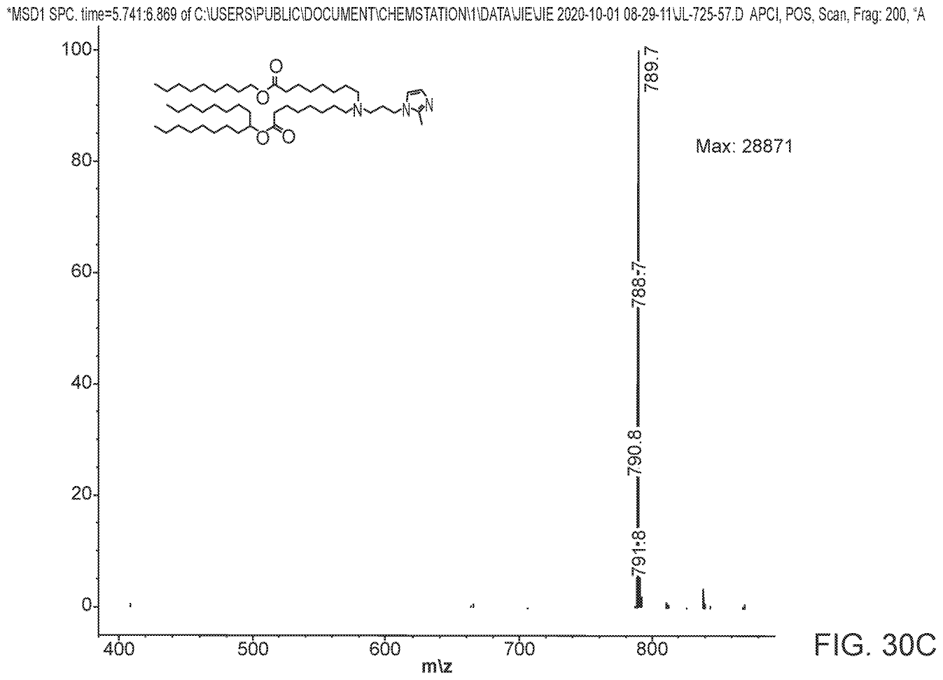

¹H NMR (ET36387-51-P1A, 400 MHz, CHLOROFORM-d) δ 0.87 (t, J=6.73 Hz, 6H) 1.25 (s, 40H) 1.33-1.40 (m, 4H) 1.52-1.61 (m, 4H) 1.81-1.90 (m, 2H) 2.36 (s, 3H) 2.39-2.46 (m, 6H) 2.53 (t, J=7.39 Hz, 4H) 2.70-2.78 (m, 8H) 3.84 (t, 0.1=7.17 Hz, 2H) 4.21 (t, J=6.95 Hz, 4H) 6.85 (s, 1H) 6.89 (s, 1H). FIG. 30 shows corresponding Nuclear Magnetic Resonance (NMR) spectrum.

### 40.3 Synthesis of bis(2-(tetradecylthio)ethyl) 3,3′-((3-(I1H-imidazol-1-yl)propyl)azanediyl)dipropionate (Lipid 93-S14)

A flask was charged with 3-(1H-imidazol-1-yl)propan-1-amine (300 mg, 2.40 mmol, 1 eq) and 2-(tetradecylthio)ethyl acrylate (1.89 g, 5.75 mmol, 2.4 eq). The neat reaction mixture was heated to 80° C. and stirred for 48 hr. TLC (ethyl acetate, R_{f}=0.3, stained by 12, one drop ammonium hydroxide added) showed the starting material was consumed completely and a new main spot was formed. The reaction mixture was diluted with dichloromethane (4 mL) and purified by column on silica gel (petroleum ether/ethyl acetate=1/20-0/100, 0.1% ammonium hydroxide added) to get bis(2-(tetradecylthio)ethyl) 3,3′-((3-(1H-imidazol-1-yl)propyl)azanediyl)dipropionate (512 mg, yield 27.22%) as colorless oil.

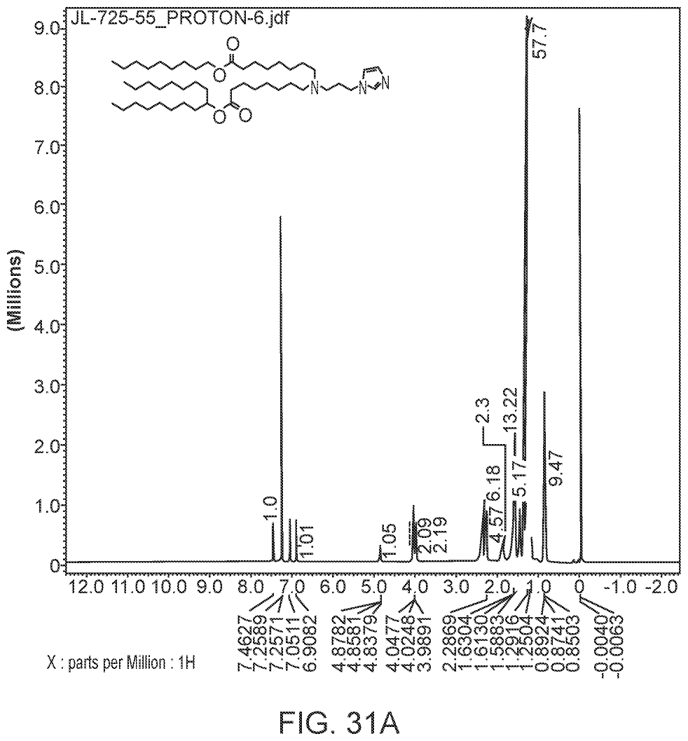

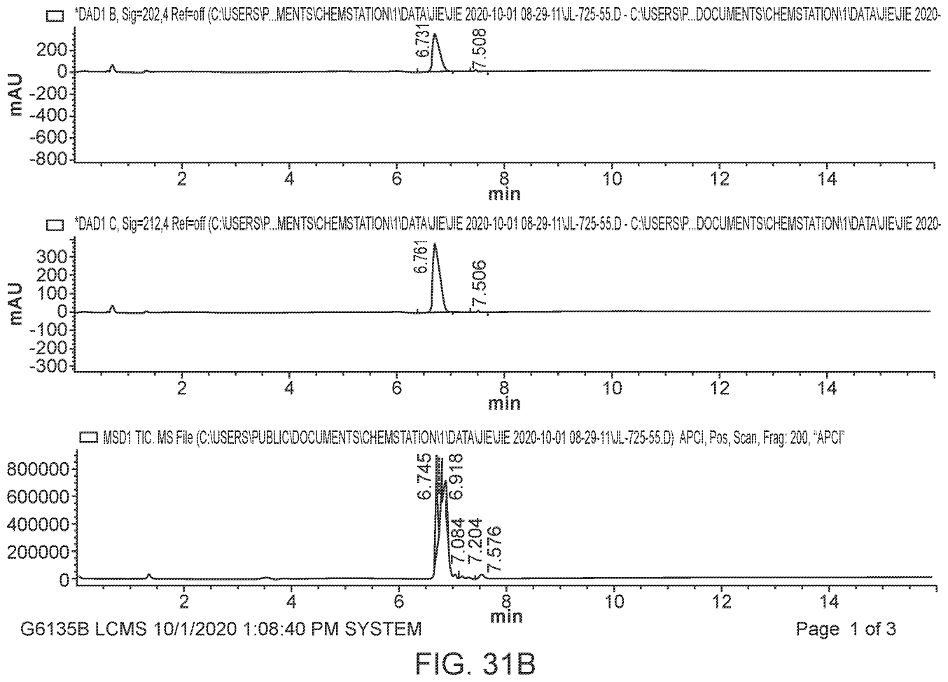

¹H NMR (ET36387-54-P1A, 400 MHz, CHLOROFORM-d) δ 0.89 (t, J=6.84 Hz, 6H) 1.26 (s, 40H) 1.34-1.41 (m, 4H) 1.58 (br t, J=7.50 Hz, 4H) 1.92 (t, J=6.62 Hz, 2H) 2.36-2.46 (m, 6H) 2.55 (t, J=7.50 Hz, 4H) 2.75 (q, J=6.84 Hz, 8H) 3.97 (t, J=6.95 Hz, 2H) 4.23 (t, J=6.95 Hz, 4H) 6.95 (s, 1H) 7.06 (s, 1H) 7.51 (s, 1H). FIG. 31 shows corresponding Nuclear Magnetic Resonance (NMR) spectrum.

### 40.4 Synthesis of heptadecan-9-yl 8-((3-(2-methyl-1H-imidazol-1-yl)propyl)(8-(nonyloxy)-8-oxooctyl)amino)octanoate (Lipid 10a-54)

### 40.4.1 Synthesis of nonyl 8-bromooctanoate (3)

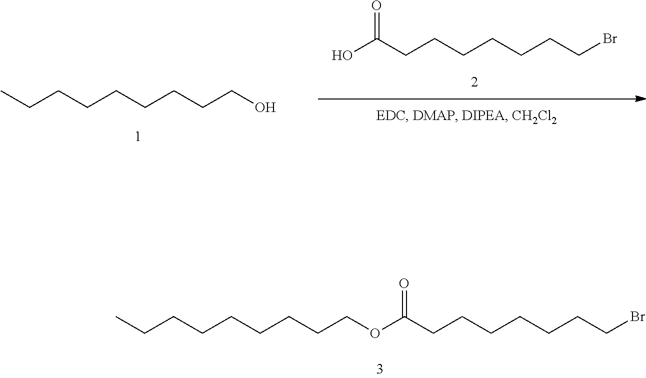

To a mixture of 8-bromooctanoic acid (2) (18.6 g, 83.18 mmol) and nonan-1-ol (1) (10 g, 69.32 mmol) in CH₂Cl₂ (500 mL) was added DMAP (1.7 g, 13.86 mmol), DIPEA (48 mL, 277.3 mmol) and EDC (16 g, 83.18 mmol). The reaction was stirred at room temperature overnight. After concentration of the reaction mixture, the crude residue was dissolved in ethyl acetate (500 mL), washed with 1N HCl, sat. NaHCO₃, water and Brine. The organic layer was dried over anhydrous Na₂SO₄. The solvent was evaporated and the crude residue was purified by flash chromatography (SiO₂:Hexane=100% to 30% of EtOAc in Hexane) and colorless oil product 3 was obtained (9 g, 37%).

### 40.4.2 Synthesis of heptadecan-9-yl 8-bromooctanoate (5)

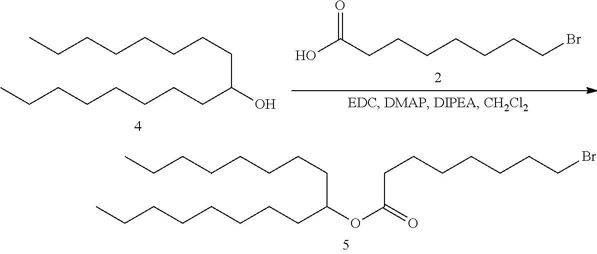 [SMILES: CCCCCCCCC(O)CCCCCCCC ; O=C(O)CCCCCCCBr ; CCCCCCCCC(CCCCCCCC)OC(=O)CCCCCCCBr]

To a mixture of 8-bromooctanoic acid (2) (10 g, 44.82 mmol) and heptadecan-9-ol (4) (9.6 g, 37.35 mmol) in CH₂Cl₂(300 mL) was added DMAP (900 mg, 7.48 mmol), DIPEA (26 mL, 149.7 mmol) and EDC (10.7 g, 56.03 mmol). The reaction was stirred at room temperature overnight. After concentration of the reaction mixture, the crude residue was dissolved in ethyl acetate (300 mL), washed with 1N HCl, sat. NaHCO₃, water and Brine. The organic layer was dried over anhydrous Na₂SO₄. The solvent was evaporated and the crude residue was purified by flash chromatography (SiO₂:Hexane=100% to 30% of EtOAc in Hexane) and colorless oil product 5 was obtained (5 g, 29%).

¹H NMR (300 MHz, CDCl₃): δ ppm 4.86 (m, 1H), 3.39 (t, J=7.0 Hz, 2H), 2.27 (t, J=7.6 Hz, 2H), 1.84 (m, 2H), 1.62 (m, 2H), 1.5-1.4 (m, 8H), 1.35-1.2 (m, 26H) 0.87 (t, J=6.7 Hz, 6H).

### 40.4.3 Synthesis of heptadecan-9-yl 8-((3-(2-methyl-1H-imidazol-1-yl)propyl)amino)octanoate (7)

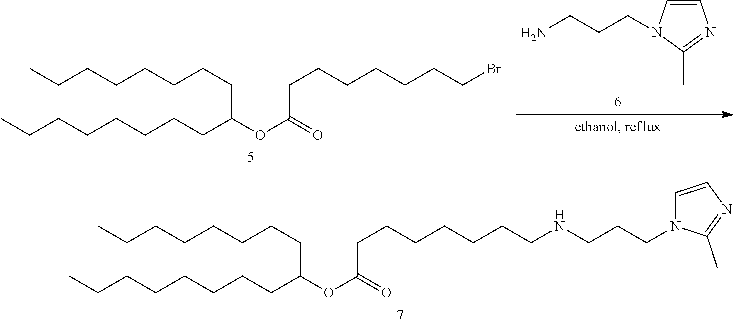

In a 100 mL round bottom flask connected with condenser, heptadecan-9-yl 8-bromooctanoate (5) (860 mg, 1.868 mmol) and 3-(2-methyl-1H-imidazol-1-yl)propan-1-amine (6) (1.3 g, 9.339 mmol) were mixed in ethanol (10 mL). The reaction mixture was heated to reflux overnight. MS (APCI) showed the expected product. The mixture was cooled to room temperature and concentrated. The crude residue was purified by flash chromatography (SiO₂:CH₂Cl₂=100% to 10% of methanol+1% NH₄OH in CH₂Cl₂) and colorless oil product 7 was obtained (665 mg, 69%).

### 40.4.4 Synthesis of heptadecan-9-yl 8-((3-(2-methyl-1H-imidazol-1-yl)propyl)(8-(nonyloxy)-8-oxooctyl)amino)octanoate (Lipid 10a-54)

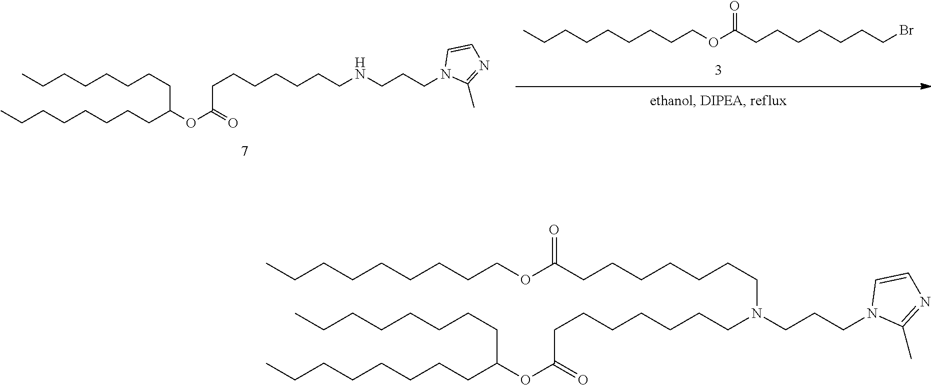 [SMILES: CCCCCCCCCOC(=O)CCCCCCCBr ; CCCCCCCCC(CCCCCCCC)OC(=O)CCCCCCCNCCCn1ccnc1C ; CCCCCCCCCOC(=O)CCCCCCCN(CCCCCCCC(=O)OC(CCCCCCCC)CCCCCCCC)CCCn1ccnc1C]

In a 100 mL round bottom flask connected with condenser, heptadecan-9-yl 8-((3-(2-methyl-1H-imidazol-1-yl)propyl)amino)octanoate (7) (665 mg, 1.279 mmol) and nonyl 8-bromooctanoate (3) (536 mg, 1.535 mmol) were mixed in ethanol (10 mL), then DIPEA (0.55 mL, 3.198 mmol) was added. The reaction mixture was heated to reflux overnight. Both MS (APCI) and TLC (10% MeOH+1% NH₄OH in CH₂Cl₂) showed the product and some unreacted starting material. The mixture was cooled to room temperature and concentrated. The crude residue was purified by flash chromatography (SiO₂:CH₂Cl₂=100% to 10% of methanol+1% NH₄OH in CH₂Cl₂) and colorless oil was obtained (170 mg, 17%).

### 40.5 Synthesis of heptadecan-9-yl 8-((3-(1H-imidazol-1-yl)propyl)(8-(nonyloxy)-8-oxooctyl)amino)octanoate (Lipid 10a-53)

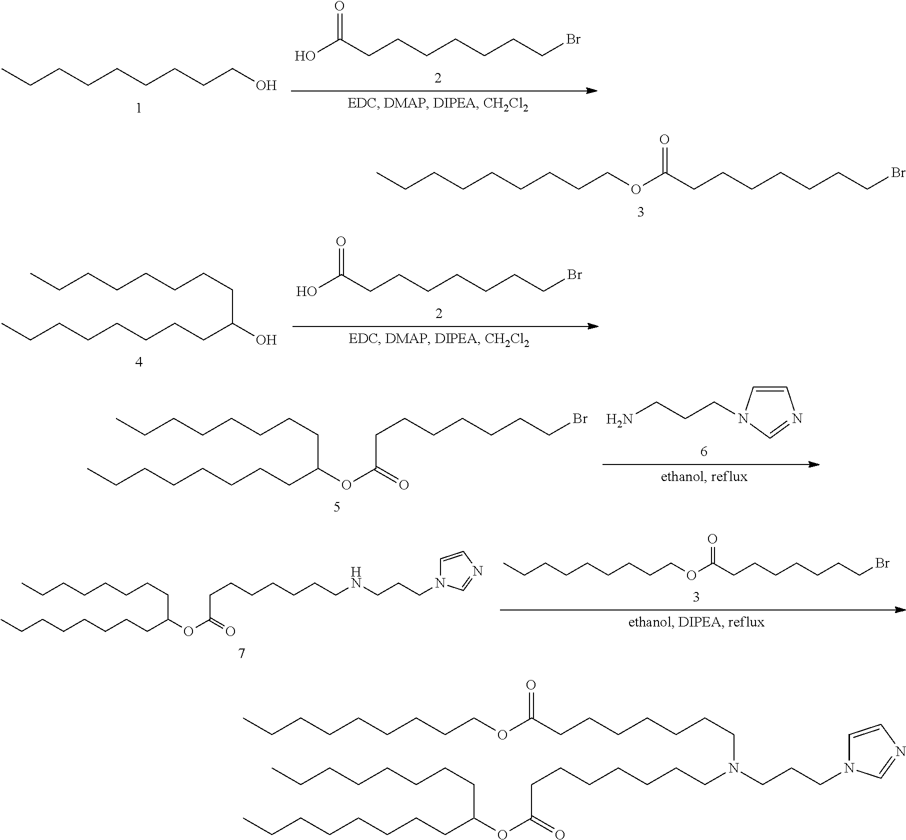

Lipid 10a-53 is synthesized according to the scheme above. Reaction conditions are identical to Lipid 10a-54 with the exception of 3-(1H-imidazol-1-yl)propan-1-amine as the imidazole amine.

### 40.6 Synthesis of Heptadecan-9-yl 8-((3-(1H-imidazol-1-yl)propyl)(8-(nonyloxy)-8-oxooctyl)amino)octanoate (Lipid 10a-45) 40.6.1 Synthesis of heptadecan-9-yl 8-bromooctanoate (3)

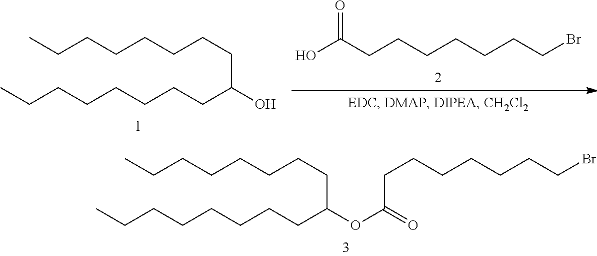 [SMILES: CCCCCCCCC(O)CCCCCCCC ; O=C(O)CCCCCCCBr ; CCCCCCCCC(CCCCCCCC)OC(=O)CCCCCCCBr]

To a mixture of 8-bromooctanoic acid (2) (10 g, 44.82 mmol) and heptadecan-9-ol (1) (9.6 g, 37.35 mmol) in CH₂Cl₂ (300 mL) was added DMAP (900 mg, 7.48 mmol), DIPEA (26 mL, 149.7 mmol) and EDC (10.7 g, 56.03 mmol). The reaction was stirred at room temperature overnight. After concentration of the reaction mixture, the crude residue was dissolved in ethyl acetate (300 mL), washed with 1N HCl, sat. NaHCO₃, water and Brine. The organic layer was dried over anhydrous Na₂SO₄. The solvent was evaporated and the crude residue was purified by flash chromatography (SiO₂:Hexane=100% to 30% of EtOAc in Hexane) and colorless oil product 3 was obtained (5 g, 29%).

¹H NMR (300 MHz, CDCl₃): δ ppm 4.86 (m, 1H), 3.39 (t, J=7.0 Hz, 2H), 2.27 (t, J=7.6 Hz, 2H), 1.84 (m, 2H), 1.62 (m, 2H), 1.5-1.4 (m, 8H), 1.35-1.2 (m, 26H) 0.87 (t, J=6.7 Hz, 6H).

### 40.6.2 Synthesis of heptadecan-9-yl 8-((3-(H-imidazol-1-yl)propyl)amino)octanoate (6)

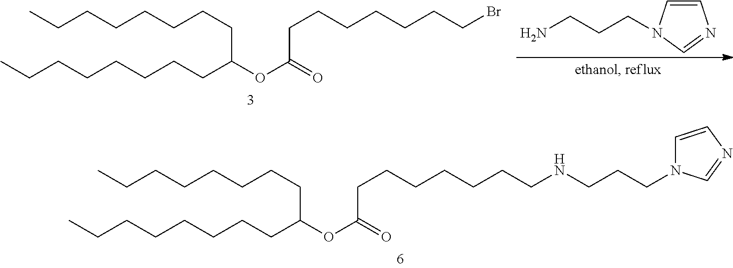 [SMILES: CCCCCCCCC(CCCCCCCC)OC(=O)CCCCCCCBr ; NCCCn1ccnc1 ; CCCCCCCCC(CCCCCCCC)OC(=O)CCCCCCCNCCCn1ccnc1]

In a 100 mL round bottom flask connected with condenser, heptadecan-9-yl 8-bromooctanoate (3) (1 g, 2.167 mmol) and 3-(1H-imidazol-1-yl)propan-1-amine (4) (1.3 mL, 10.83 mmol) were mixed in ethanol (10 mL). The reaction mixture was heated to reflux overnight. MS (APCI) showed the expected product. The mixture was cooled to room temperature and concentrated. The crude residue was purified by flash chromatography (SiO₂:CH₂Cl₂=100% to 10% of methanol+1% NH40H in CH₂Cl₂) and colorless oil product 6 was obtained (498 mg, 45%).

¹H NMR (300 MHz, CDCl₃): δ ppm 7.47 (s, 1H), 7.04 (s, 1H), 6.91 (s, 1H), 4.85 (m, 1H), 4.03 (t, J=7.0 Hz, 2H), 2.56 (dd, J=14.5, 7.4 Hz, 4H), 2.27 (t, J=7.4 Hz, 2H), 1.92 (m, 2H), 1.60 (m, 2H), 1.48 (m, 6H), 1.30-1.20 (m, 31H), 0.86 (t, J=6.6 Hz, 6H). MS (APCI⁺): 506.4 (M+1).

### 40.6.3 Synthesis of nonyl 8-bromooctanoate (9)

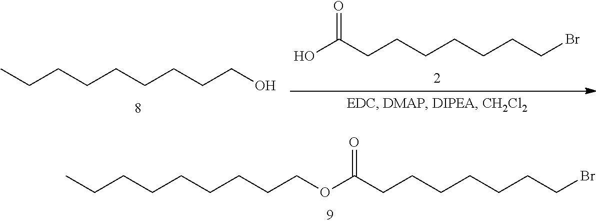

To a mixture of 8-bromooctanoic acid (2) (18.6 g, 83.18 mmol) and nonan-1-ol (8) (10 g, 69.32 mmol) in CH2Cl₂ (500 mL) was added DMAP (1.7 g, 13.86 mmol), DIPEA (48 mL, 277.3 mmol) and EDC (16 g, 83.18 mmol). The reaction was stirred at room temperature overnight. After concentration of the reaction mixture, the crude residue was dissolved in ethyl acetate (500 mL), washed with 1N HCl, sat. NaHCO₃, water and Brine. The organic layer was dried over anhydrous Na2SO₄. The solvent was evaporated and the crude residue was purified by flash chromatography (SiO₂:Hexane=100% to 30% of EtOAc in Hexane) and colorless oil product 9 was obtained (9 g, 37%).

¹H NMR (300 MHz, CDCl₃): δ ppm 4.05 (t, J=7.0 Hz, 2H), 3.39 (t, 0.1=7.0 Hz, 2H), 2.29 (t, J=7.6 Hz, 2H), 1.84 (m, 2H), 1.62-1.56 (m, 6H), 1.40-1.20 (m, 16H), 0.87 (t, J=6.7 Hz, 3H). 40.6.4 Synthesis of heptadecan-9-yl 8-((3-(III-imidazol-J-yl)propyl)(8-(nonyloxy)-8-oxooctyl)amino)octanoate

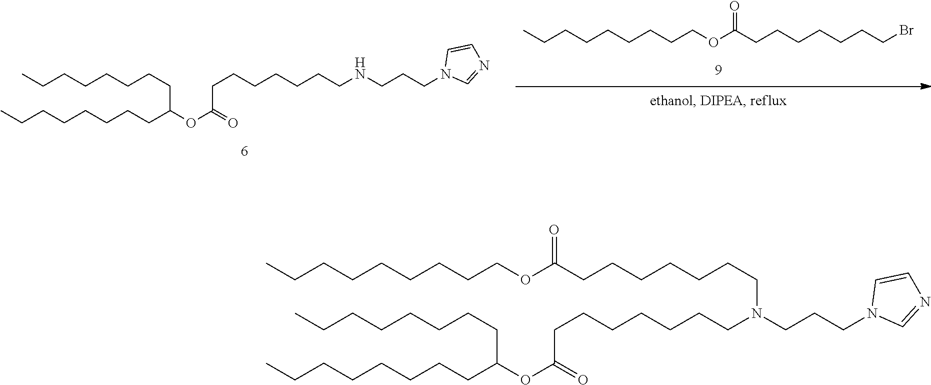 [SMILES: CCCCCCCCCOC(=O)CCCCCCCBr ; CCCCCCCCC(CCCCCCCC)OC(=O)CCCCCCCNCCCn1ccnc1 ; CCCCCCCCCOC(=O)CCCCCCCN(CCCCCCCC(=O)OC(CCCCCCCC)CCCCCCCC)CCCn1ccnc1]

In a 100 mL round bottom flask connected with condenser, heptadecan-9-yl 8-((3-(1H-imidazol-1-yl)propyl)amino)octanoate (6) (242 mg, 0.478 mmol) and nonyl 8-bromooctanoate 9 (200 mg, 0.574 mmol) were mixed in ethanol (10 mL), then DIPEA (0.2 mL, 1.196 mmol) was added. The reaction mixture was heated to reflux overnight. Both MS (APCI) and TLC (10% MeOH+1% NH40H in CH₂Cl₂) showed the product and some unreacted starting material. The mixture was cooled to room temperature and concentrated. The crude residue was purified by flash chromatography (SiO₂:CH₂Cl₂=100% to 10% of methanol+1% NH₄OH in CH₂Cl₂) and colorless oil was obtained (35 mg, 10%).

¹H NMR (300 MHz, CDCl3): δ ppm 7.46 (s, 1H), 7.05 (s, 1H), 6.90 (s, 1H), 4.85 (m, 1H), 4.04 (t, J=6.6 Hz, 2H), 4.01 (t, J=6.6 Hz, 2H), 2.38 (m, 6H), 2.27 (t, J=3.8 Hz, 4H), 1.89 (m, 2H), 1.60-1.58 (m, 12H), 1.48 (m, 6H), 1.30-1.20 (m, 47H), 0.87 (t, J=7.1 Hz, 9H). MS (APCI+): 774.6 (M+1).

### 40.7 Synthesis of Heptadecan-9-yl 8-((3-(2-methyl-H-imidazol-1 yl)propyl)(8-(nonyloxy)-8-oxooctyl)amino)octanoate (Lipid 10a-46) 0

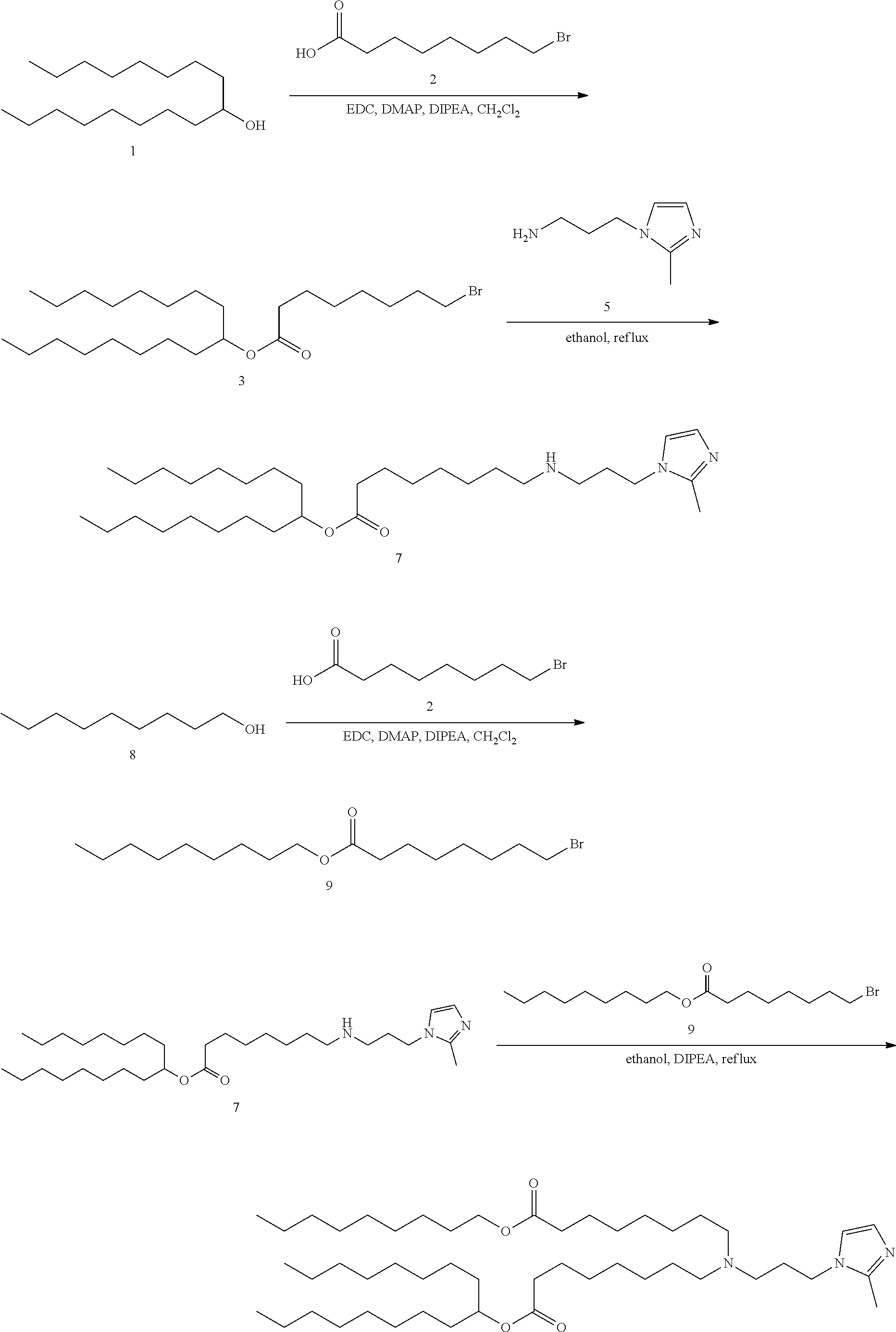

Lipid 10a-46 is synthesized according to the scheme above. Reaction conditions are identical to Lipid 10a-45 with the exception of 3-(2-Methyl-1H-imidazol-1-yl)propan-1-amine as the imidazole amine.

¹H NMR (300 MHz, CDCl₃): δ ppm 6.89 (s, 1H), 6.81 (s, 1H), 4.86 (m, 1H), 4.04 (t, J=6.8 Hz, 2H), 3.85 (t, J=7.4 Hz, 2H), 2.38-2.36 (m, 9H), 2.28 (m, 4H), 1.82 (m, 2H), 1.72-1.56 (m, 12H), 1.48 (m, 4H), 1.30-1.20 (m, 46H), 0.86 (t, J=6.6 Hz, 9H). MS (APCI-): 789.7 (M+1).

### 40.8 Synthesis of Heptadecan-9-yl 8-((3-(1H-imidazol-1-yl)propyl)(8-oxo-8-(unidocan-3-yloxy)octyl)amino)octanoate (Lipid 10a-137)

### 40.9.1, Synthesis of heptadecan-9-yl 8-bromooctanoate (3)

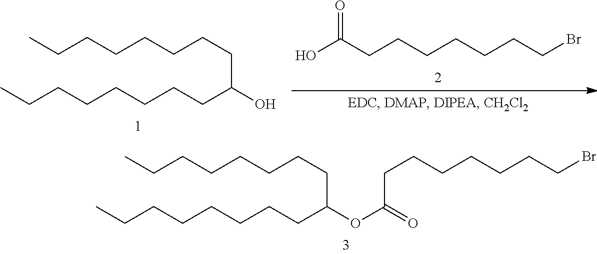 [SMILES: CCCCCCCCC(O)CCCCCCCC ; O=C(O)CCCCCCCBr ; CCCCCCCCC(CCCCCCCC)OC(=O)CCCCCCCBr]

To a mixture of 8-bromooctanoic acid (2) (10 g, 44.82 mmol) and heptadecan-9-ol (1) (9.6 g, 37.35 mmol) in CH₂Cl₂ (300 mL) was added DMAP (900 mg, 7.48 mmol), DIPEA (26 mL, 149.7 mmol) and EDC (10.7 g, 56.03 mmol). The reaction was stirred at room temperature overnight. After concentration of the reaction mixture, the crude residue was dissolved in ethyl acetate (300 mL), washed with 1N HCl, sat. NaHCO₃, water and Brine. The organic layer was dried over anhydrous Na₂SO₄. The solvent was evaporated and the crude residue was purified by flash chromatography (SiO₂:Hexane=100% to 30% of EtOAc in Hexane) and colorless oil product 3 was obtained (5 g, 29%).

1H NMR (300 MHz, CDCl3): δ ppm 4.86 (m, 1H), 3.39 (t, J=7.0 Hz, 2H), 2.27 (t, J=7.6 Hz, 2H), 1.84 (m, 2H), 1.62 (m, 2H), 1.5-1.4 (m, 8H), 1.35-1.2 (m, 26H) 0.87 (t, J=6.7 Hz, 6H).

### 40.2 Synthesis of heptadecan-9-yl 8-((3-(1H-imidazol-1-yl)propyl)amino)octanoate (6)

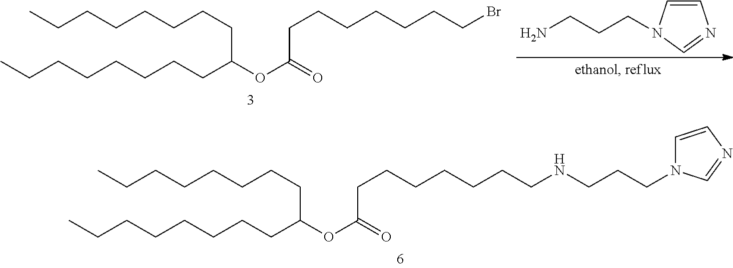 [SMILES: CCCCCCCCC(CCCCCCCC)OC(=O)CCCCCCCBr ; NCCCn1ccnc1 ; CCCCCCCCC(CCCCCCCC)OC(=O)CCCCCCCNCCCn1ccnc1]

In a 100 mL round bottom flask connected with condenser, heptadecan-9-yl 8-bromooctanoate (3) (1 g, 2.167 mmol) and 3-(1H-imidazol-1-yl)propan-1-amine (4) (1.3 mL, 10.83 mmol) were mixed in ethanol (10 mL). The reaction mixture was heated to reflux overnight. MS (APCI) showed the expected product. The mixture was cooled to room temperature and concentrated. The crude residue was purified by flash chromatography (SiO₂:CH₂Cl₂'₂ 100% to 10¹,% of methanol+1% NH₄OH in CH₂Cl₂) and colorless oil product 6 was obtained (498 mg, 45%).

¹H NMR (300 MHz, CDCl₃): δ ppm 7.47 (s, 1H), 7.04 (s, 1H), 6.91 (s, 1H), 4.85 (m, 1H), 4.03 (t, J=7.0 Hz, 2H), 2.56 (dd, 1=14.5, 7.4 Hz, 4H), 2.27 (t, 1=7.4 Hz, 2H), 1.92 (m, 2H), 1.60 (m, 2H), 1.48 (m, 6H), 1.30-1.20 (m, 31H), 0.86 (t, J=6.6 Hz, 6H). MS (APCI⁺): 506.4 (M+1).

### 40.83 Synthesis of undecan-3-ol (11)

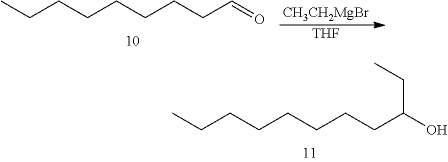

To a mixture of nonanal (10) (5 g, 35.2 mmol), in anhydrous THF (100 mL) at 0° C. ice-water bath was dropwise added ethylmagnesium bromide (47 mL, 42.2 mmol, 0.9M in THF). The reaction was stirred at room temperature overnight. The reaction was quenched with ice and diluted with ethyl acetate (500 mL), washed with 1N HCl, sat. NaHCO₃, water and Brine. The organic layer was dried over anhydrous Na₂SO₄. The solvent was evaporated and the crude residue was purified by flash chromatography (SiO₂:Hexane=100% to 50% of EtOAc in Hexane) and colorless oil product 11 was obtained (4 g, 66%).

¹H NMR (300 MHz, CDCl₃): δ ppm 3.52 (m, 1H), 1.56-1.3 (m, 4H), 1.3-1.20 (m, 12H), 0.93 (t, J=7.4 Hz, 3H), 0.87 (t, J=7.4 Hz, 3H).

### 40.8.4 Synthesis of undecan-3-yl 8-bromooctanoate (12)

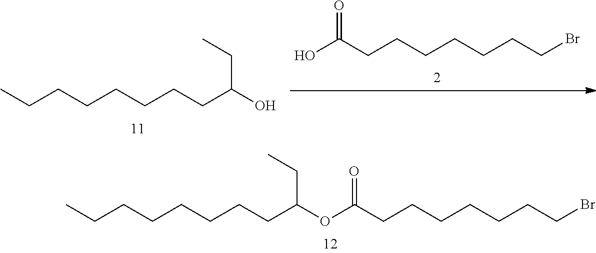 [SMILES: CCCCCCCCC(O)CC ; O=C(O)CCCCCCCBr ; CCCCCCCCC(CC)OC(=O)CCCCCCCBr]

To a mixture of 8-bromooctanoic acid (2) (6.2 g, 27.9 mmol) and undecan-3-ol (11) (4 g, 23.2 mmol) in CH₂Cl₂ (100 mL) was added DMAP (567.2 mg, 4.64 mmol), DIPEA (16.2 mL, 92.9 mmol) and EDC (6.7 g, 34.8 mmol). The reaction was stirred at room temperature overnight. After concentration of the reaction mixture, the crude residue was dissolved in ethyl acetate (500 mL), washed with 1N HCl, sat. NaHCO₃, water and Brine. The organic layer was dried over anhydrous Na₂SO₄. The solvent was evaporated and the crude residue was purified by flash chromatography (SiO₂:Hexane=100% to 30% of EtOAc in Hexane) and colorless oil product 12 was obtained (7.3 g, 83%).

¹H NMR (300 MHz, CDCl₃): δ ppm 4.80 (m, 1H), 3.39 (t, J=6.8 Hz, 2H), 2.28 (t, J=7.7 Hz, 2H), 1.84 (m, 2H), 1.6-1.35 (m, 8H), 1.35-1.2 (m, 16H), 0.87 (t, J=7.4 Hz, 6H).

### 40.8.4 Synthesis of heptadecan-9-yl 8-((3-(1H-imidazol-1-yl)propyl)(8-(nonyloxy)-8-oxooctyl)amino)octanoate

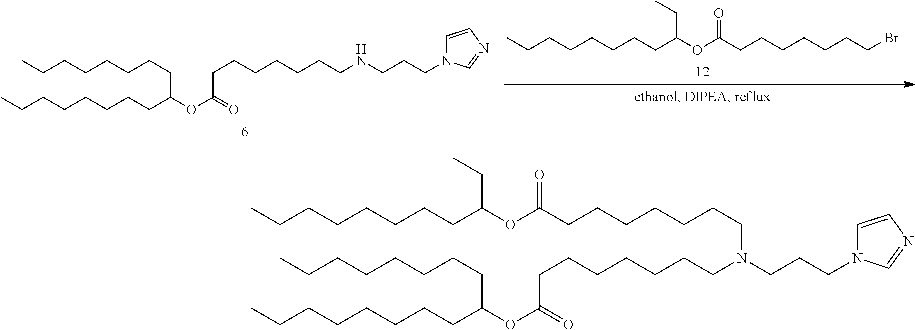

In a 100 mL round bottom flask connected with condenser, heptadecan-9-yl 8-((3-(1H-imidazol-1-yl)propyl)amino)octanoate (6) (242 mg, 0.478 mmol) and undecan-3-yl 8-bromooctanoate (12) (200 mg, 0.574 mmol) were mixed in ethanol (10 mL), then DIPEA (0.2 mL, 1.196 mmol) was added. The reaction mixture was heated to reflux overnight. Both MS (APCI) and TLC (10% MeOH+1% NH₄OH in CH₂Cl₂) showed the product and some unreacted starting material. The mixture was cooled to room temperature and concentrated. The crude residue was purified by flash chromatography (SiO₂:CH₂Cl₂=100% to 10% of methanol+1% NH₄OH in CH₂Cl₂) and colorless oil was obtained (35 mg, 10%).

¹H NMR (300 MHz, CDCl3): δ ppm 7.45 (s, 1H), 7.04 (s, 1H), 6.90 (s, 1H), 4.82 (m, 2H), 3.97 (t, J=6.8 Hz, 2H), 2.35 (m, 6H), 2.27 (t, J=3.8 Hz, 4H), 1.89 (m, 2H), 1.60-1.48 (m, 14H), 1.30-1.20 (m, 50H), 0.87 (m, 12H). MS (APCI+): 802.8

### 40.9 Synthesis of Heptadecan-9-yl 8-((3-(2-methyl-H-imidazol-1-yl)propyl)(8-(nonyloxy)-8-oxooctyl)amino)octanoate (Lipid 10a-138)

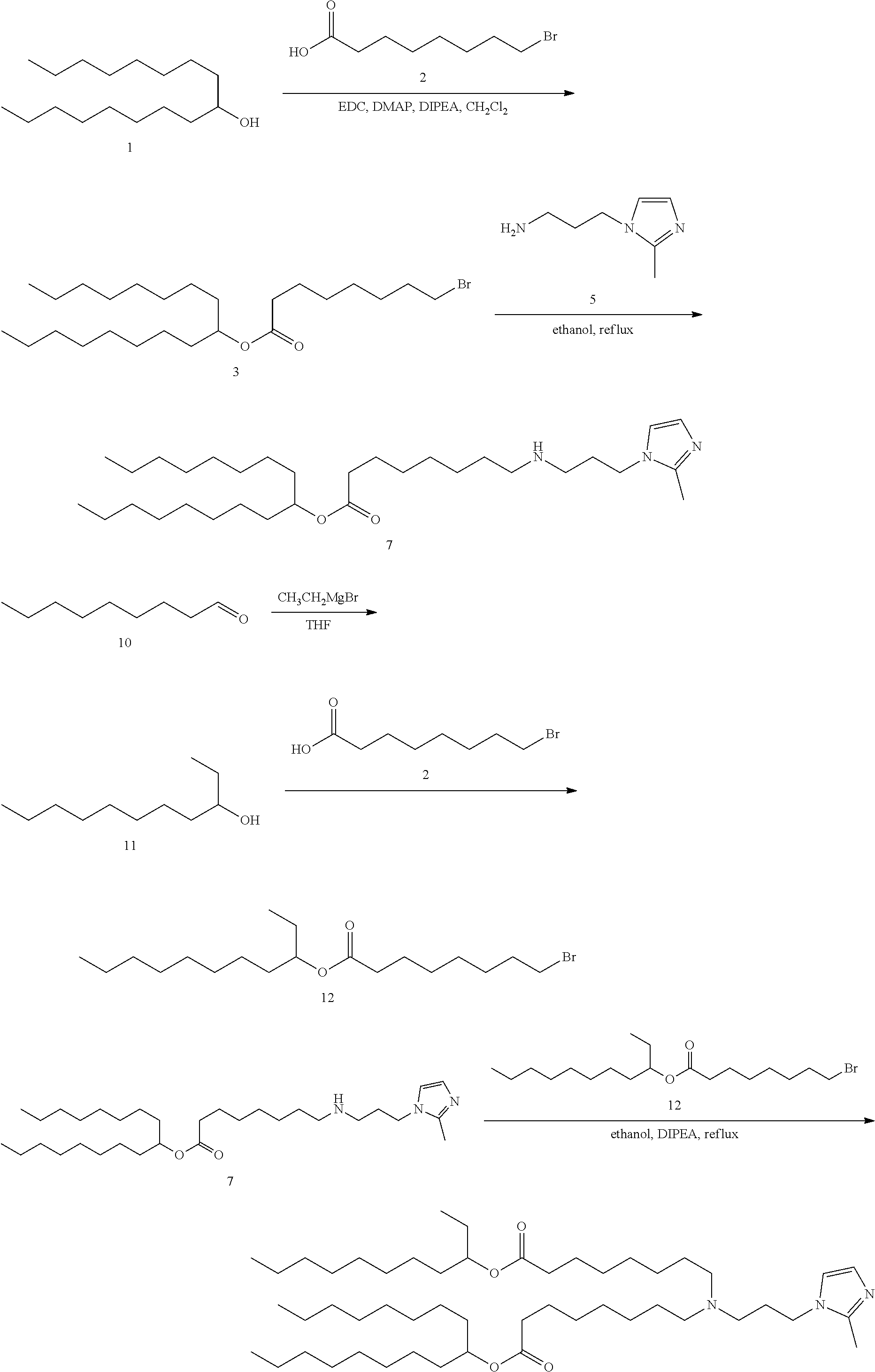

Lipid 10a-138 is synthesized according to the scheme above. Reaction conditions are identical to Lipid 10a-137 with the exception of 3-(2-Methyl-1H-imidazol-1-yl)propan-1-amine as the imidazole amine.

¹H NMR (300 MHz, CDCl3): δ ppm 6.89 (s, 1H), 6.81 (s, 1H), 4.82 (m, 2H), 3.86 (t, J=7.1 Hz, 2H), 2.38-2.3 (m, 9H), 2.27 (t, J=3.8 Hz, 4H), 1.84 (m, 2H), 1.60-1.37 (m, 14H), 1.30-1.20 (m, 50H), 0.87 (m, 12H). MS (APCI+): 816.8 (M+1).

### 40.10 Synthesis of (((2-(2-Methyl-1H-imidazol-1-yl)ethyl)azanediyl)bis(hexane-6,1-diyl) bis(2-hexyldecanoate (Lipid 10a-139)

### 40.10.1 Synthesis of 6-bromohexyl 2-hexyldecanoate (3)

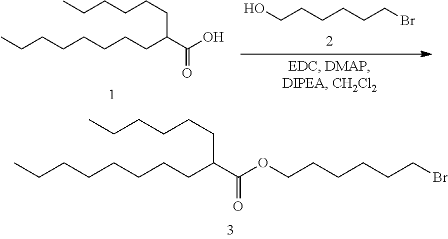 [SMILES: CCCCCCCCC(CCCCCC)C(=O)O ; OCCCCCCBr ; CCCCCCCCC(CCCCCC)C(=O)OCCCCCCBr]

To a mixture of 2-hexyldecanoic acid (1) (102 g, 0.398 mol) and 6-bromo-1-hexanol (2) (60 g, 0.331 mol) in CH₂Cl₂ (1 L) was added DMAP (8.1 g, 66 mmol), DIPEA (230 mL, 1.325 mol) and EDC (76 g, 0.398 mol). The reaction was stirred at room temperature overnight. After concentration of the reaction mixture, the crude residue was dissolved in ethyl acetate (1 L), washed with 1N HCl, sat. NaHCO₃, water and Brine. The organic layer was dried over anhydrous Na₂SO₄. The solvent was evaporated and the crude residue was purified by flash chromatography (SiO₂:Hexane=100% to 30% of EtOAc in Hexane) and colorless oil product 3 was obtained (67 g, 48%).

¹H NMR (300 MHz, CDCl3): δ ppm 4.06 (t, J=6.6 Hz, 2H), 3.4 (t, J=6.8 Hz, 2H), 2.3 (m, 1H), 1.86 (m, 2H), 1.64 (m, 2H), 1.5-1.4 (m, 2H), 1.35-1.2 (m, 26H) 0.87 (t, J=6.7 Hz, 6H).

### 40.10.2 Synthesis of 6-((3-(1H-imidazol-1-yl)butyl)amino)hexyl 2-hexyldecanoate (7a)

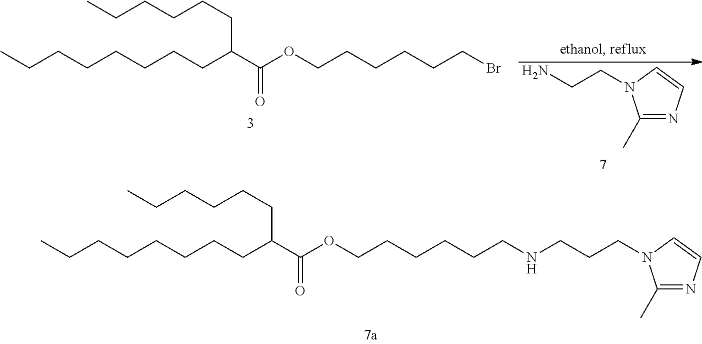

In a 100 mL round bottom flask connected with condenser, 6-bromohexyl 2-hexyldecanoate (3) (1.2 g, 2.87 mmol) and 3-(1H-imidazol-1-yl)butan-1-amine (7) (2 g, 14.37 mmol) were mixed in ethanol (20 mL). The reaction mixture was heated to reflux overnight. MS (APCI) showed the expected product. The mixture was cooled to room temperature and concentrated. The crude residue was purified by flash chromatography (SiO₂:CH₂Cl₂=100% to 10% of methanol+1% NH40H in CH₂Cl₂) and colorless oil product 7a was obtained (626 mg, 46%).

¹H NMR (300 MHz, CDCl₃): b ppm 7.51 (s, 1H), 7.05 (s, 1H), 6.93 (s, 1H), 4.35 (m, 1H), 4.04 (t, J=6.6 Hz, 2H), 2.6-2.4 (m, 4H), 2.29 (m, 1H), 1.94 (td, J=14, 6.8 Hz, 2H), 1.64-1.56 (m, 4H), 1.47 (s, 3H), 1.42-1.20 (m, 29H), 0.86 (m, 6H). MS (APCI⁺): 478.8 (M+1)

### 40.10.2 Synthesis of ((2-(2-Methyl-1H-imidazol-1-yl)ethyl)azanediyl)bis(hexane-6,1-diyl) bis(2-hexyldecanoate

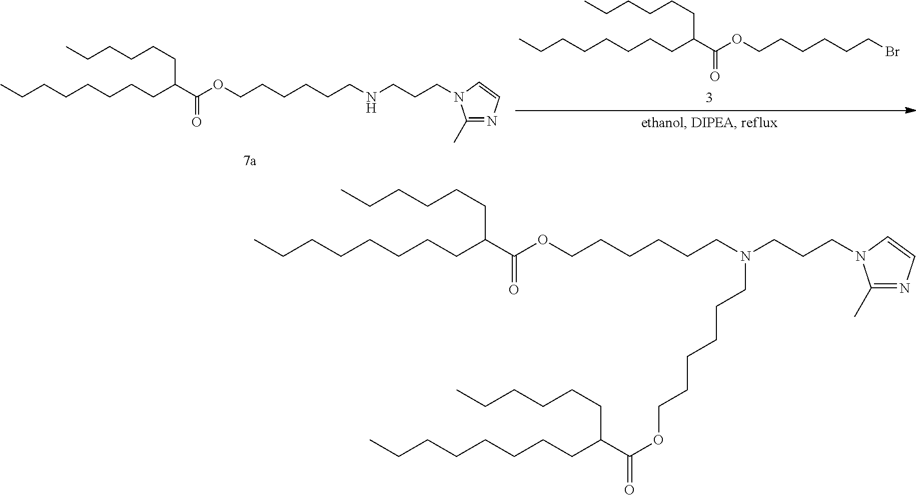 [SMILES: CCCCCCCCC(CCCCCC)C(=O)OCCCCCCNCCCn1ccnc1C ; CCCCCCCCC(CCCCCC)C(=O)OCCCCCCBr ; CCCCCCCCC(CCCCCC)C(=O)OCCCCCCN(CCCCCCOC(=O)C(CCCCCC)CCCCCCCC)CCCn1ccnc1C]

In a 100 mL round bottom flask connected with condenser, 6-((3-(1H-imidazol-1-yl)butyl)amino)hexyl 2-hexyldecanoate (7a) (626 mg, 1.31 mmol) and 6-bromohexyl 2-hexyldecanoate (3) (550 mg, 1.31 mmol) were mixed in ethanol (20 mL), then DIPEA (0.6 mL, 3.276 mmol) was added. The reaction mixture was heated to reflux overnight. Both MS (APCI) and TLC (10% MeOH+1% NH₄OH in CH₂Cl₂) showed the product and unreacted starting material 7a. The mixture was cooled to room temperature and concentrated. The crude residue was purified by flash chromatography (SiO₂:CH₂Cl₂=100% to 10% of methanol+1% NH40H in CH₂Cl₂) and the obtained product was further purified by C18 reverse phase chromatography (H₂O=95% to 0.1% TFA in CH₃CN=100%) colorless oil (TFA salt) was obtained (140 mg, 13%).

¹H-NMR (300 MHz, CDCl₃): δ 6.87 (s, 1H), 6.83 (s, 1H), 4.05 (t, J=6.7 Hz, 4H), 3.84 (t, J=6.9 Hz, 2H), 2.66 (t, J=6.9 Hz, 2H), 2.45-2.20 (m, 6H), 2.37 (s, 3H), 1.65-1.50 (m, 8H), 1.5-1.1 (m, 56H), 0.86 (t, J=6.5 Hz, 12H). MS (APCI⁺): 802.6 (M+1).

### 40.11 Synthesis of (((1-Methyl-1H-imidazo-2-yl)methyl)azanediyl)bis(hexane-6,1-diyl) bis(2-hexyldecanoate) (Lipid 10a-130)

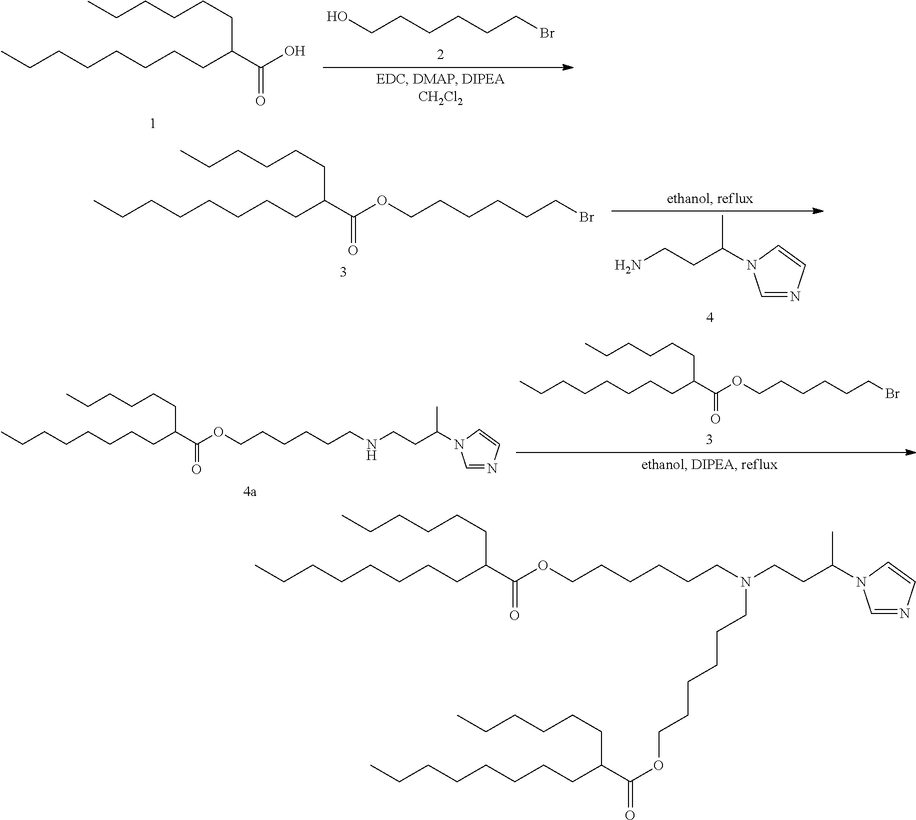

Lipid 10a-130 is synthesized according to the scheme above. Reaction conditions are identical to Lipid 10a-139 with the exception of 3-(1H-imidazol-1-yl)butyl amine as the imidazole amine.

### 40.12 Synthesis of (((1-Methyl-1H-imidazol-2-yl)methyl)azanediyl)bis(hexane-6,1-diyl) bis(2-hexyldecanoate) (Lipid 10a-128)

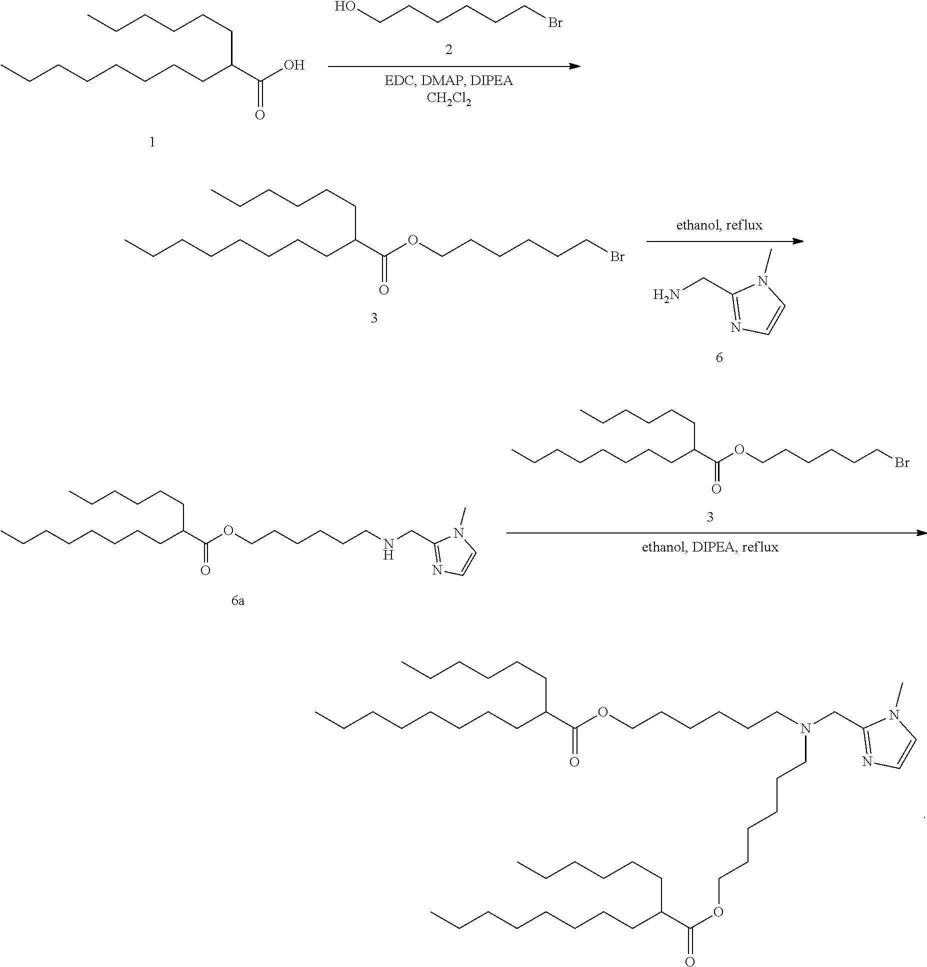

Lipid 10a-128 is synthesized according to the scheme above. Reaction conditions are identical to Lipid 10a-139 with the exception of 1-Methyl-1H-imidazol-2-yl)methyl amine as the imidazole amine.

¹H-NMR (300 MHz, CDCl₃): δ 6.89 (d, J=1.4 Hz, 1H), 6.81 (d, J=1.4 Hz, 1H), 4.03 (t, J=6.7 Hz, 4H), 3.68 (s, 3H), 3.62 (s, 2H), 2.45-2.20 (m, 6H), 1.65-1.50 (m, 8H), 1.5-1.35 (m, 8H), 1.35-1.10 (m, 48H), 0.86 (t, J=6.5 Hz, 12H). MS (APCI⁺): 787.6 (M+1).

### Example 41

Lipid Nanoparticle Formulation with Circular RNA

Lipid Nanoparticles (LNPs) were formed using a Precision Nanosystems Ignite instrument with a ‘NextGen’ mixing chamber. Ethanol phase contained ionizable Lipid 10a-26, DSPC, Cholesterol, and DSPE-PEG 2000 (Avanti Polar Lipids Inc.) at a weight ratio of 16:1:4:1 or 62:4:33:1 molar ratio was combined with an aqueous phase containing circular RNA and 25 mM sodium acetate buffer at pH 5.2. A 3:1 aqueous to ethanol mixing ratio was used. The formulated LNP then were dialyzed in 1 L of water and exchanged 2 times over 18 hours. Dialyzed LNPs were filtered using 0.2 pm filter. Prior to in vivo dosing, LNPs were diluted in PBS. LNP sizes were determined by dynamic light scattering. A cuvette with 1 mL of 20 μg/mL LNPs in PBS (pH 7.4) was measured for Z-average using the Malvern Panalytical Zetasizer Pro. The Z-average and polydispersity index were recorded.

41.1 Formulation of Lipids 10a-26 and 10a-27

Lipid Nanoparticles (LNPs) were formed using a Precision Nanosystems Ignite instrument with a ‘NextGen’ mixing chamber. Ethanol phase contained ionizable Lipid 10a-26 or Lipid 10a-27, DOPE, Cholesterol, and DSPE-PEG 2000 (Avanti Polar Lipids Inc.) at a weight ratio of 16:1:4:1 or 62:4:33:1 molar ratio was combined with an aqueous phase containing circular RNA and 25 mM sodium acetate buffer at pH 5.2. A 3:1 aqueous to ethanol mixing ratio was used. The formulated LNPs were then dialyzed in 1 L of water and exchanged 2 times over 18 hours. Dialyzed LNPs were filtered using 0.2 pm filter. Prior to in vivo dosing, LNPs were diluted in PBS. LNP sizes were determined by dynamic light scattering. A cuvette with 1 mL of 20 μg/mL LNPs in PBS (pH 7.4) was measured for Z-average using the Malvern Panalytical Zetasizer Pro. The Z-average and polydispersity index were recorded.

39.2 Formulation of Lipids 10a-53 and 10a-54

Lipid Nanoparticles (LNPs) were formed using a Precision Nanosystems Ignite instrument with a ‘NextGen’ mixing chamber. Ethanol phase contained ionizable Lipid 10a-53 or 10a-54, DOPE, Cholesterol, and DSPE-PEG 2000 (Avanti Polar Lipids Inc.) at a molar ratio of 50:10:38.5:1.5 was combined with an aqueous phase containing circular RNA and 25 mM sodium acetate buffer at pH 5.2. A 3:1 aqueous to ethanol mixing ratio was used. The formulated LNPs were then dialyzed in 1 L of 1×PBS and exchanged 2 times over 18 hours. Dialyzed LNPs were filtered using 0.2 pm filter. Prior to in vivo dosing, LNPs were diluted in PBS. LNP sizes were determined by dynamic light scattering. A cuvette with 1 mL of 20 μg/mL LNPs in PBS (pH 7.4) was measured for Z-average using the Malvern Panalytical Zetasizer Pro. The Z-average and polydispersity index were recorded.

LNP zeta potential was measured using the Malvern Panalytical Zetasizer Pro. A mixture containing 200 μL of the particle solution in water and 800 μL of distilled RNAse-free water with a final particle concentration of 400 μg/mL was loaded into a zetasizer capillary cell for analysis.

RNA encapsulation was determined using a Ribogreen assay. Nanoparticle solutions were diluted in tris-ethylenediaminetetraacetic acid (TE) buffer at a theoretical circRNA concentration of 2 μg/mL. Standard circRNA solutions diluted in TE buffer were made ranging from 2 μg/mL to 0.125 μg/mL. The particles and standards were added to all wells and a second incubation was performed (37° C. at 350 rpm for 3 minutes). Fluorescence was measured using a SPECTRAmax® GEMINI XS microplate spectrofluorometer. The concentration of circular RNA in each particle solution was calculated using the standard curve. The encapsulation efficiency was calculated from the ratio of circRNA detected between lysed and unlysed particles.

TABLE 16a
Characterization of LNPs

| | | | Encapsulation | Zeta Data |
|---|---|---|---|---|
| Ionizable Lipid | Size (nm) | PDI | Efficiency (%) | Potential (mV) |
| 22-S14 | 88 | 0.09 | 96 | 3.968 |
| 93-S14 | 119 | 0.02 | 96 | −6.071 |
| Lipid 10a-26 | 86 | 0.08 | 92 | −15.24 |

TABLE 16b
Characterization of LNPs

| Ionizable Lipid | Z-Average(nm) | PDI | RNA Entrapment(%) |
|---|---|---|---|
| 22-S14 | 64 | 0.05 | 97 |
| 93-S14 | 74 | 0.04 | 95 |
| Lipid 10a-26 | 84 | 0.04 | 96 |

### In Vivo Analysis

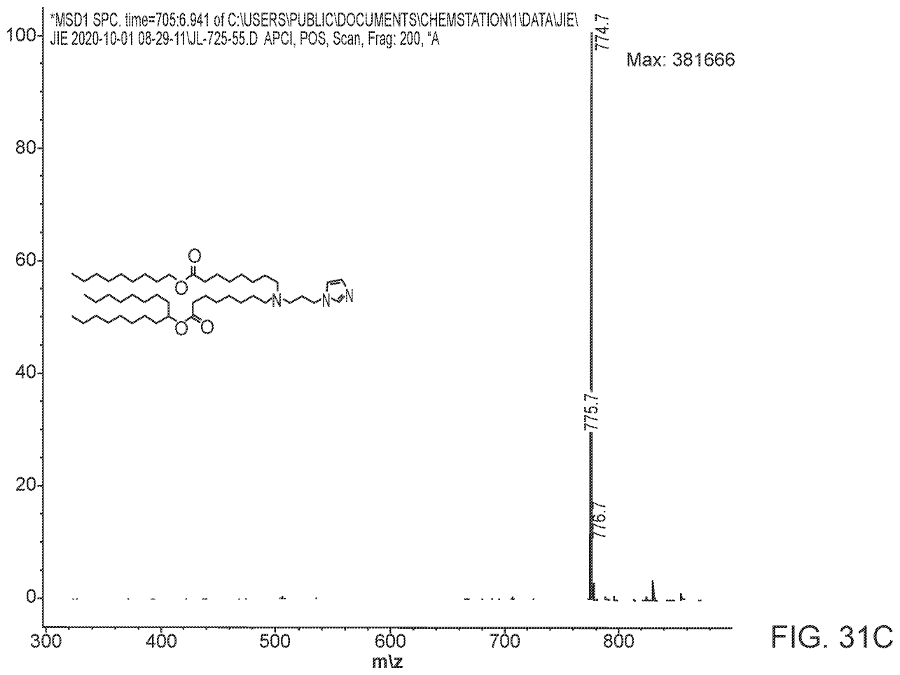

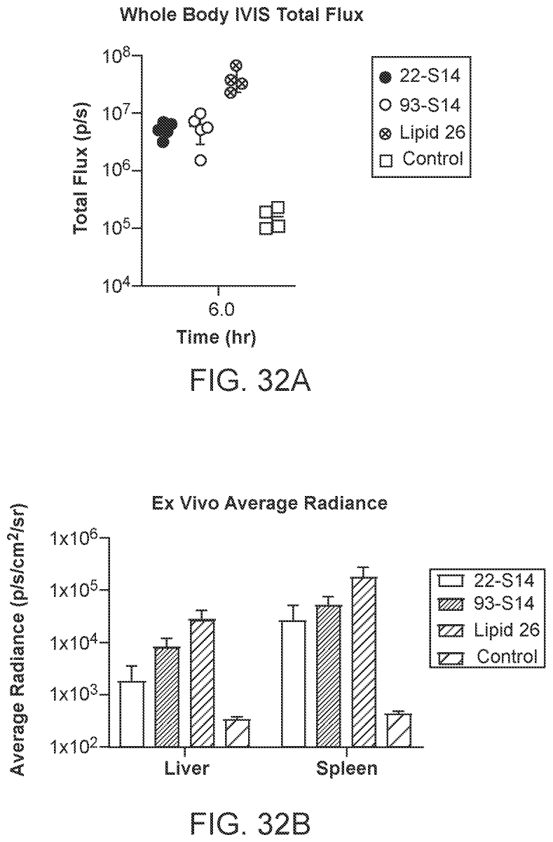

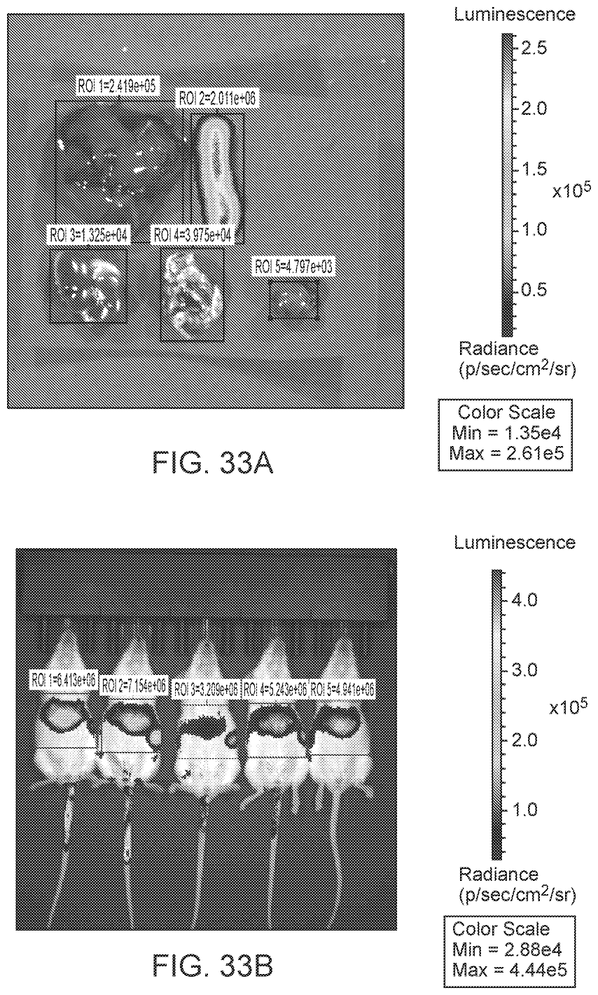

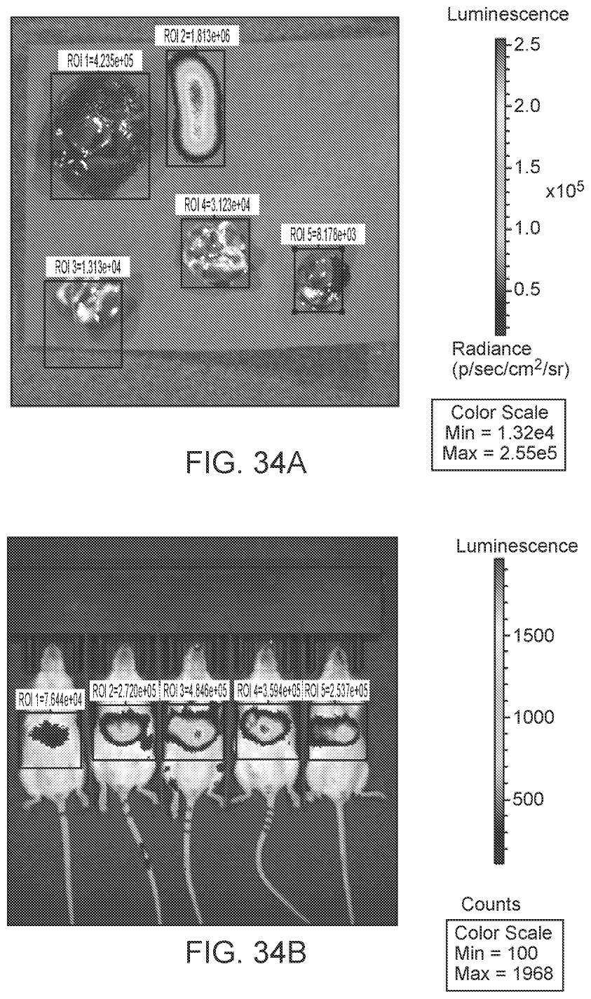

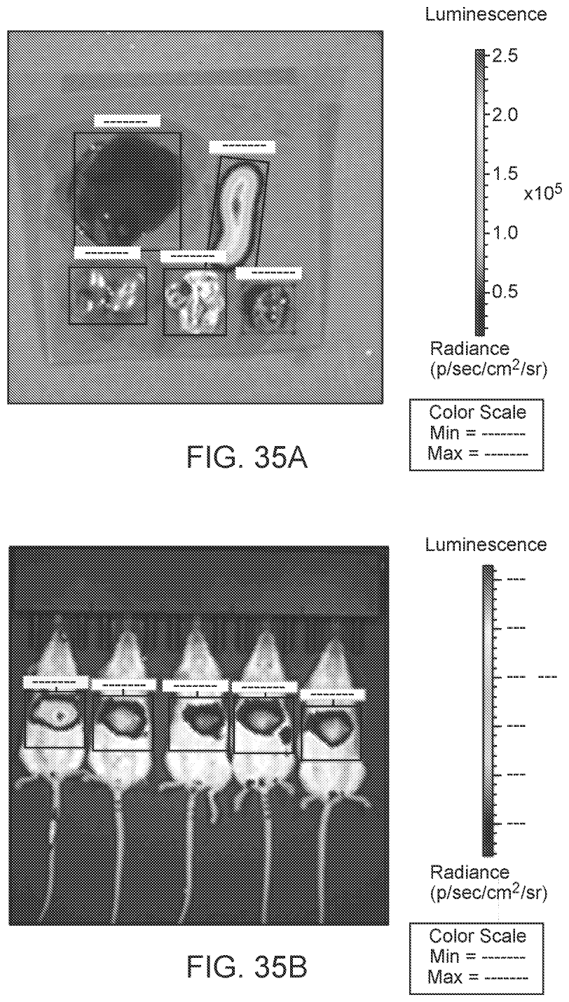

Female CD-1 or female c57BL/6J mice ranging from 22-25 g were dosed at 0.5 mg/kg RNA intravenously. Six hours after injection, mice were injected intraperitoneally with 200 μL of D-luciferin at 15 mg/mL concentration. 5 minutes after injection, mice were anesthetized using isoflurane, and placed inside the IVIS Spectrum In Vivo Imaging System (Perkin Elmer) with dorsal side up. Whole body total IVIS flux of Lipids 22-S14, 93-S14, Lipid 10a-26 is presented in FIG. 32A. Post 10 minutes injection, mice were scanned for luminescence. Mice were euthanized and organs were extracted within 25 minutes of luciferin injection to scan for luminescence in liver, spleen, kidneys, lungs, and heart. Images (FIGS. 33A-B, 34A-B, 35A-B) were analyzed using Living Images (Perkin Elmer) software. Regions of interest were drawn to obtain flux and average radiance and analyzed for biodistribution of protein expression (FIG. 32A-B).

FIG. 32A illustrates the increased whole-body total flux observed from luciferase circRNA with Lipid 10a-26 LNPs compared to LNPs made with lipids 22-S14 and 93-S14. FIG. 32B shows the ex vivo IVIS analysis of tissues further highlighting the increased overall expression with Lipid 10a-26 while maintaining the desired spleen to liver ratios observed with lipids 22-S14 and 93-S14 despite the significant structural changes designed to improve expression. These data highlight the improvements afforded by Lipid 10a-26 compared to previously reported lipids.

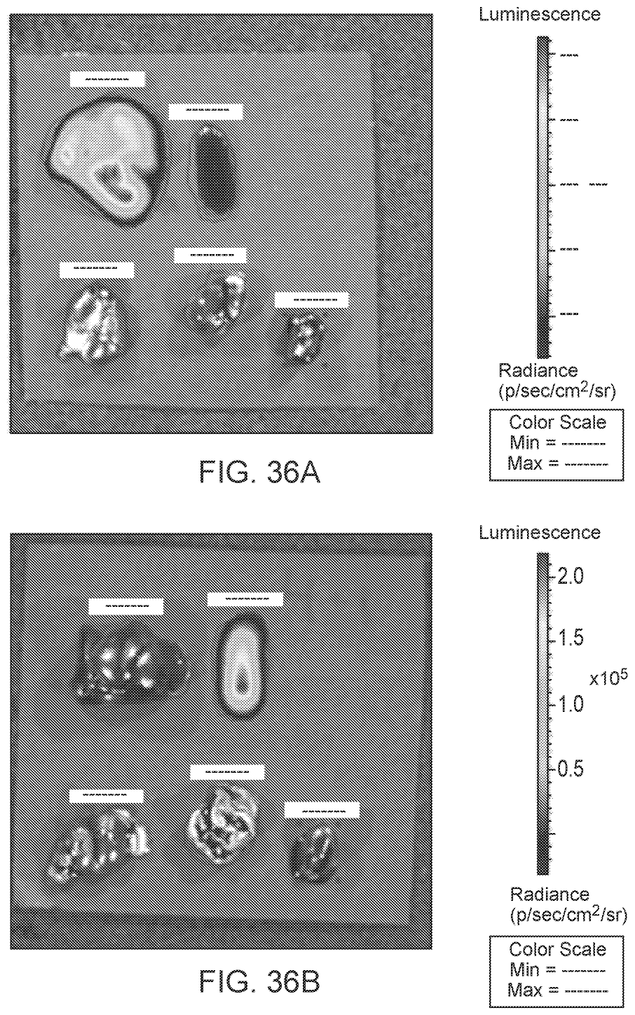

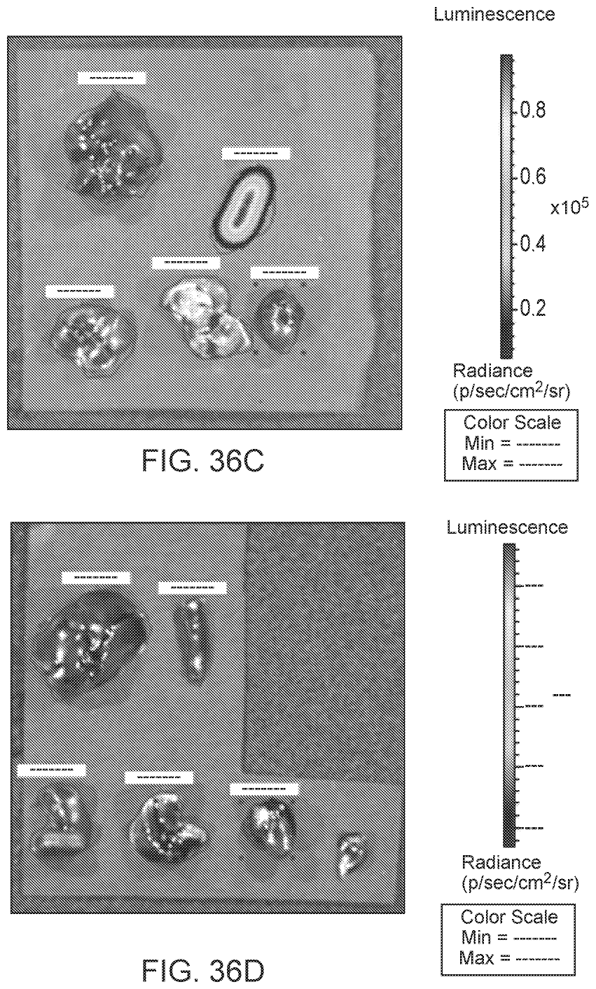

Similar analysis as described above was also performed with circRNA encapsulated in LNPs formed with Lipid 10b-15 or Lipid 10a-53 or 10a-54. FIGS. 36A-C show the ex vivo IVIS analysis of tissues, respectively highlighting the overall expression with Lipid 10b-15, 10a-53, and 10a-54 while maintaining the desired spleen to liver ratios despite the significant structural changes designed to improve expression. FIG. 36D shows the results for PBS control. These data demonstrates the improvements afforded by Lipids 10b-15, 10a-53, and 10a-54 compared to previously reported lipids such as 93-S14 and 22-S14.

### Example 43

### Delivery of Luciferase

Human peripheral blood mononuclear cells (PBMCs) (Stemcell Technologies) were transfected with lipid nanoparticles (LNP) encapsulating firefly luciferase (f.luc) circular RNA and examined for luciferase expression. PBMCs from two different donors were incubated in vitro with five different LNP compositions, containing circular RNA encoding for firefly luciferase (200 ng), at 37° C. in RPMI, 2% human serum, IL-2 (10 ng/mL), and 50 uM BME. PBMCs incubated without LNP were used as a negative control. After 24 hours, the cells were lysed and analyzed for firefly luciferase expression based on bioluminescence (Promega BrightGlo).

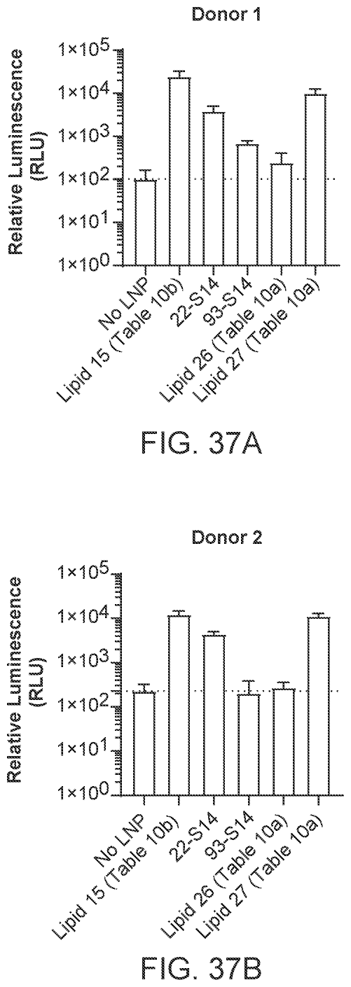

Representative data are presented in FIGS. 37A and 37B, showing that that the tested LNPs are capable of delivering circular RNA into primary human immune cells resulting in protein expression.

### Example 44

### In Vitro Delivery of Green Fluorescent Protein (GFP) or Chimeric Antigen Receptor (CAR)

Human PBMCs (Stemcell Technologies) were transfected with LNP encapsulating GFP and examined by flow cytometry. PBMCs from five different donors (PBMC A-E) were incubated in vitro with one LNP composition, containing circular RNA encoding either GFP or CD19-CAR (200 ng), at 37° C. in RPMI, 2% human serum, IL-2 (10 ng/mL), and 50 uM BME. PBMCs incubated without LNP were used as a negative control. After 24, 48, or 72 hours post-LNP incubation, cells were analyzed for CD3, CD19, CD56, CD14, CD11 b, CD45, fixable live dead, and payload (GFP or CD19-CAR).

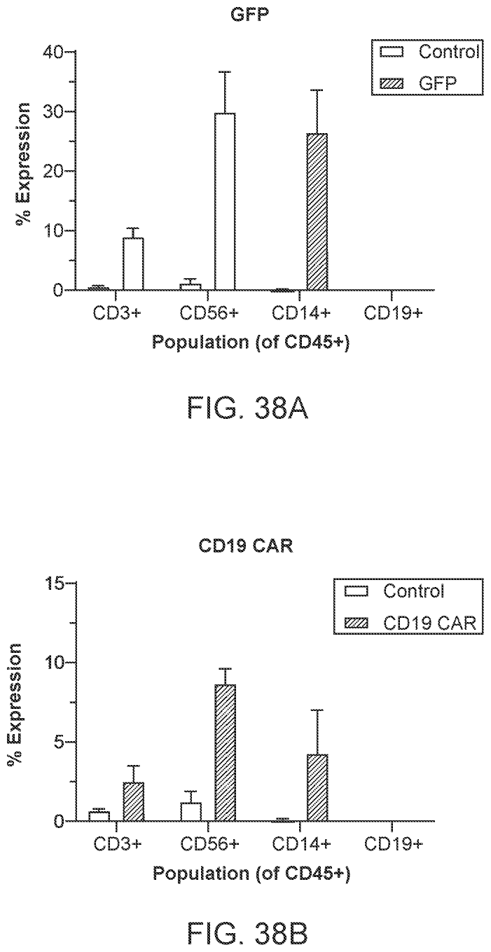

Representative data are presented in FIGS. 38A and 38B, showing that the tested LNP is capable of delivering circular RNA into primary human immune cells resulting in protein expression.

### Example 45

### Multiple IRES Variants can Mediate Expression of Murine CD19 CAR In Vitro

Multiple circular RNA constructs, encoding anti-murine CD19 CAR, contains unique IRES sequences and were lipotransfected into 1C1C7 cell lines. Prior to lipotransfection, 1C1C7 cells are expanded for several days in complete RPMI Once the cells expanded to appropriate numbers, 1C1C7 cells were lipotransfected (Invitrogen RNAiMAX) with four different circular RNA constructs. After 24 hours, 1C1C7 cells were incubated with His-tagged recombinant murine CD19 (Sino Biological) protein, then stained with a secondary anti-His antibody. Afterwards, the cells were analyzed via flow cytometry.

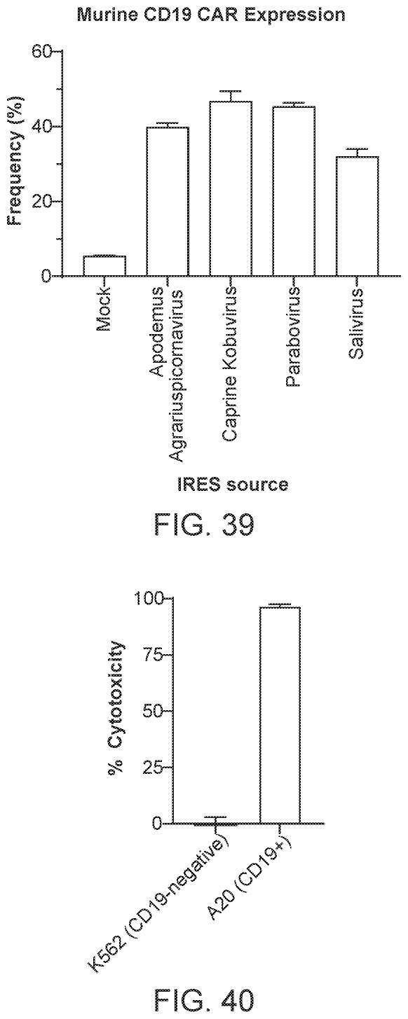

Representative data are presented in FIGS. 39, showing that IRES sourced from the indicated virus (apodemus agrarius picornavirus, caprine kobuvirus, parabovirus, and salivirus) are capable of driving expression of an anti-mouse CD19 CAR in murine T cells.

### Example 46

### Murine CD 19 CAR Mediates Tumor Cell Killing In Vitro

Circular RNA encoding anti-mouse CD19 CAR were electroporated into murine T cells to evaluate CAR-mediated cytotoxicity. For electroporation, T cells were electroporated with circular RNA encoding anti-mouse CD19 CAR using ThermoFisher's Neon Transfection System then rested overnight. For the cytotoxicity assay, electroporated T cells were co-cultured with Fluc+ target and non-target cells at 1:1 ratio in complete RPMI containing 10% FBS, IL-2 (10 ng/mL), and 50 uM BME and incubated overnight at 37° C. Cytotoxicity was measured using a luciferase assay system 24 hours post-co-culture (Promega Brightglo Luciferase System) to detect lysis of Fluc+ target and non-target cells. Values shown are calculated relative to the untransfected mock signal.

Representative data are presented in FIG. 40, showing that an anti-mouse CD19 CAR expressed from circular RNA is functional in murine T cells in vitro.

### Example 47

Functional Depletion of B Cells with a Lipid Encapsulated Circular RNA Encoding Murine CD19 CAR

C57BL/6J mice were injected with LNP formed with Lipid 10b-15, encapsulating circular RNA encoding anti-murine CD19 CAR. As a control, Lipid 10b-15 encapsulating circular RNA encoding firefly luciferase (fLuc) were injected in different group of mice. Female C57BL.6J, ranging from 20-25 g, were injected intravenously with 5 doses of 0.5 mg/kg of LNP, every other day. Between injections, blood draws were analyzed via flow cytometry for fixable live/dead, CD45, TCRvb, B220, CD11b, and anti-murine CAR. Two days after the last injection, spleens were harvested and processed for flow cytometry analysis. Splenocytes were stained with fixable live/dead, CD45, TCRvb, B220, CD11b, NK1.1, F4/80, CD11c, and anti-murine CAR. Data from mice injected with anti-murine CD19 CAR LNP were normalized to mice that received f.Luc LNP.

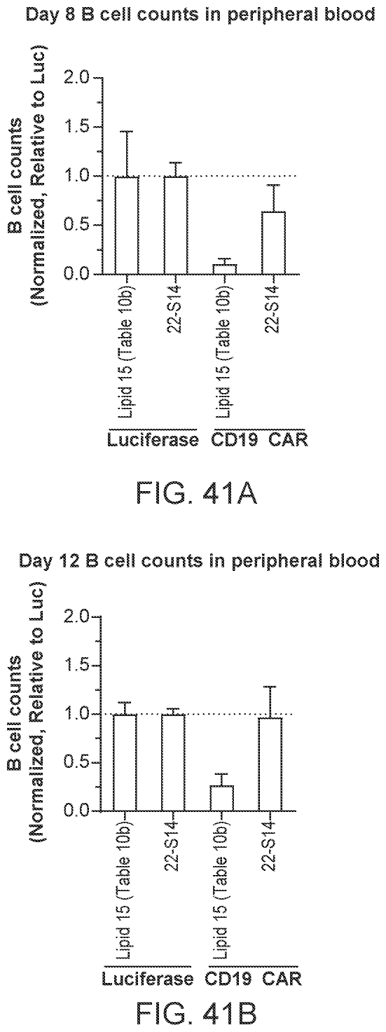

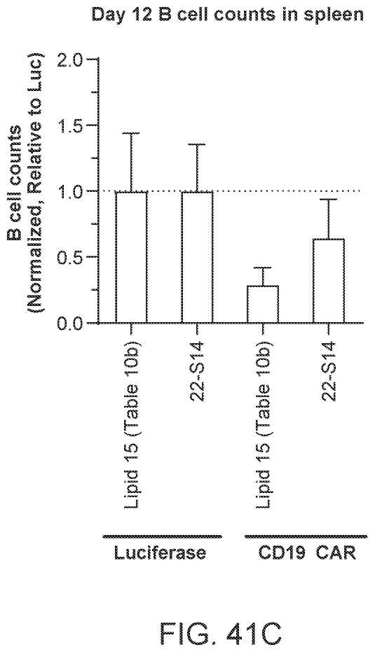

Representative data are presented in FIGS. 41A, 41B, and 41C, showing that an anti-mouse CD19 CAR expressed from circular circRNA delivered in vivo with LNPs is functional in murine T cells in vivo.

### Example 48

CD19 CAR Expressed from Circular RNA has Higher Yield and Greate Cytotoxic Effect Compared to that Expressed from mRNA

Circular RNA encoding encoding anti-CD19 chimeric antigen antigen receptor, which includes, from N-terminus to C-terminus, a FMC63-derived scFv, a CD8 transmembrane domain, a 4-1BB costimulatory domain, and a CD3(intracellular domain, were electroporated into human peripheral T cells to evaluate surface expression and CAR-mediated cytotoxicity. For comparison, circular RNA-electroporated T cells were compared to mRNA-electroporated T cells in this experiment. For electroporation, CD3+ T cells were isolated from human PBMCs using commercially available T cell isolation kits (Miltenyi Biotec) from donor human PBMCs. After isolation, T cells were stimulated with anti-CD3/anti-CD28 (Stemcell Technologies) and expanded over 5 days at 37° C. in complete RPMI containing 10¹,% FBS, IL-2 (10 ng/mL), and 50 uM BME. Five days post stimulation, T cells were electroporated with circular RNA encoding anti-human CD19 CAR using ThermoFisher's Neon Transfection System and then rested overnight. For the cytotoxicity assay, electroporated T cells were co-cultured with Fluc+ target and non-target cells at 1:1 ratio in complete RPMI containing 10% FBS, IL-2 (10 ng/mL), and 50 uM BME and incubated overnight at 37° C. Cytotoxicity was measured using a luciferase assay system 24 hours post-co-culture (Promega Brightglo Luciferase System) to detect lysis of Fluc+ target and non-target cells. Furthermore, an aliquot of electroporated T cells were taken and stained for live dead fixable staining, CD3, CD45, and chimeric antigen receptors (FMC63) at the day of analysis.

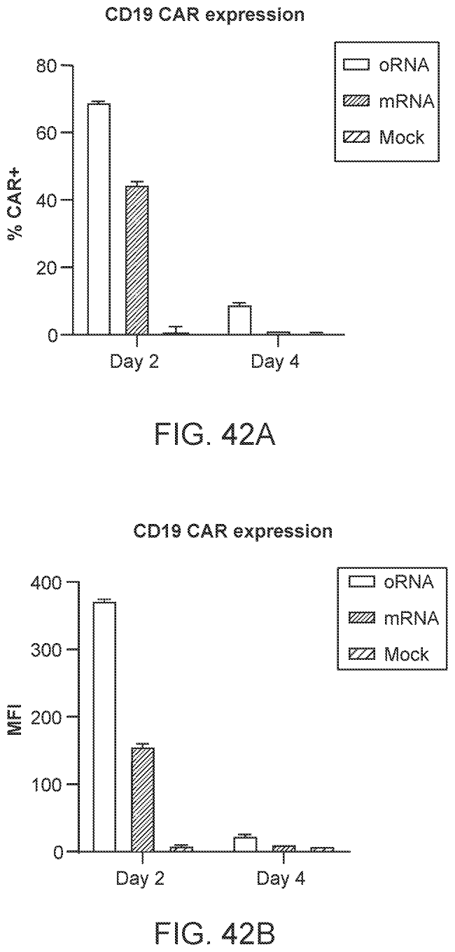

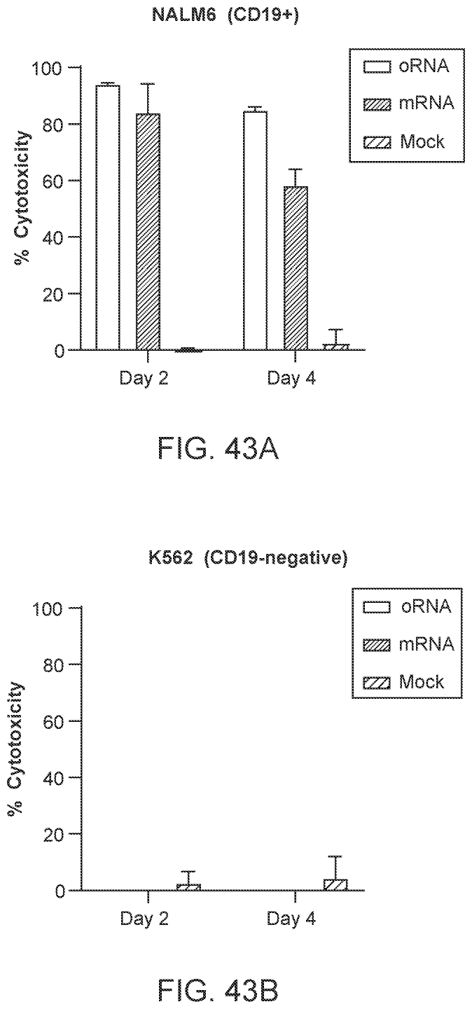

Representative data are presented in FIGS. 42 and 43. FIGS. 42A and 42B show that an anti-human CD19 CAR expressed from circular RNA is expressed at higher levels and longer than an anti-human CD19 CAR expressed from linear mRNA. FIGS. 43A and 43B show that an anti-human CD19 CAR expressed from circular RNA is exerts a greater cytotoxic effect relative a to anti-human CD19 CAR expressed from linear mRNA.

### Example 49

Functional Expression of Two CARs from a Single Circular RNA

Circular RNA encoding chimeric antigen receptors were electroporated into human peripheral T cells to evaluate surface expression and CAR-mediated cytotoxicity. The purpose of this study is to evaluate if circular RNA encoding for two CARs can be stochastically expressed with a 2A (P2A) or an LRES sequence. For electroporation, CD3+ T cells were commercially purchased (Cellero) and stimulated with anti-CD3/anti-CD28 (Stemcell Technologies) and expanded over 5 days at 37° C. in complete RPMI containing 10% FBS, IL-2 (10 ng/mL), and 50 uM BME. Four days post stimulation, T cells were electroporated with circular RNA encoding anti-human CD19 CAR, anti-human CD19 CAR-2A-anti-human BCMA CAR, and anti-human CD19 CAR-IRES-anti-human BCMA CAR using ThermoFisher's Neon Transfection System then rested overnight. For the cytotoxicity assay, electroporated T cells were co-cultured with Fluc+K562 cells expressing human CD19 or BCMA antigens at 1:1 ratio in complete RPMI containing 10% FBS, IL-2 (10 ng/mL), and 50 uM BME and incubated overnight at 37° C. Cytotoxicity was measured using a luciferase assay system 24 hours post-co-culture (Promega BrightGlo Luciferase System) to detect lysis of Fluc+ target cells.

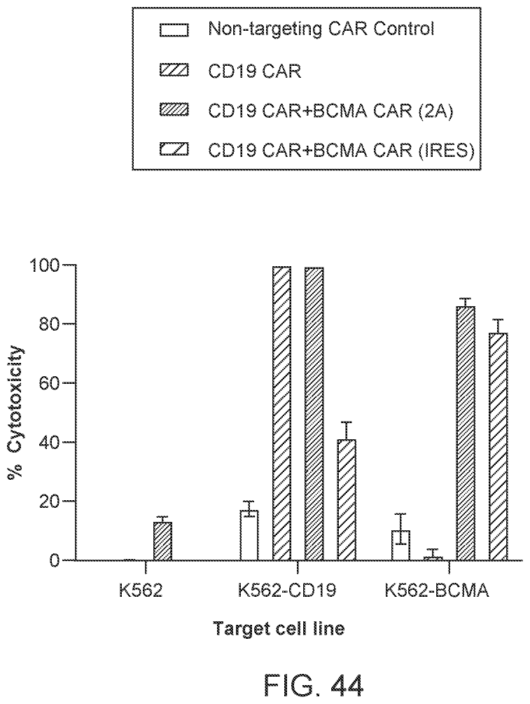

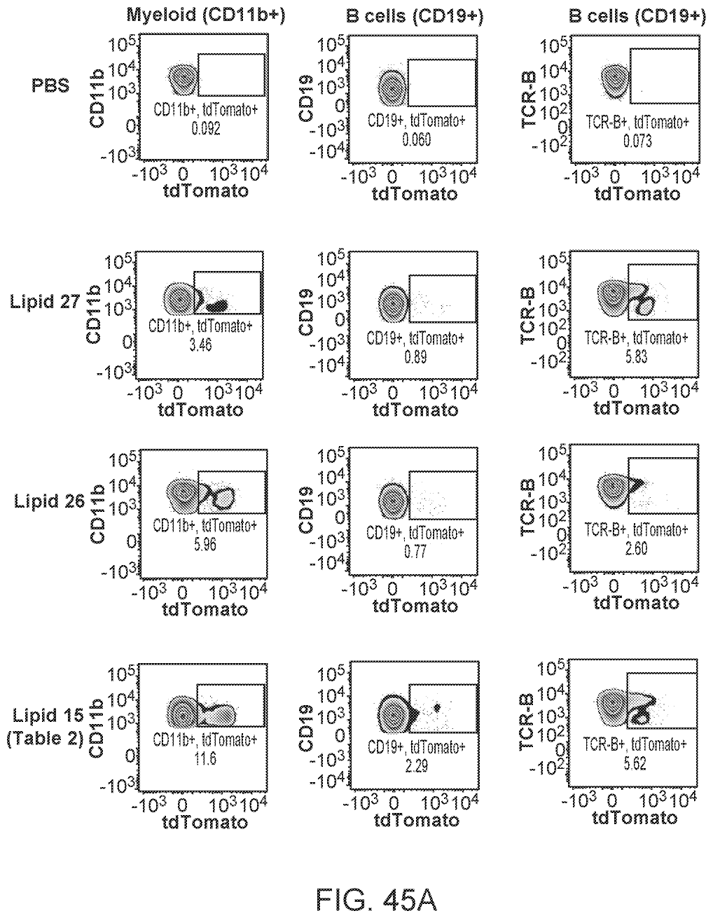

Representative data are presented in FIG. 44, showing that two CARs can be functionally expressed from the same circular RNA construct and exert cytotoxic effector function.

### Example 50

### In Vivo Circular RNA Transfection Using Cre Reporter Mice

Circular RNAs encoding Cre recombinase (Cre) are encapsulated into lipid nanoparticles as previously described. Female, 6-8 week old B6.Cg-Gt(ROSA)26Sortm9(CAG-tdTomato)Hze/J (Ai9) mice were dosed with lipid nanoparticles at 0.5 mg/kg RNA intravenously. Fluorescent tdTomato protein was transcribed and translated in Ai9 mice upon Cre recombination, meaning circular RNAs have been delivered to and translated in tdTomato+ cells. After 48 hr, mice were euthanized and the spleens were harvested, processed into a single cell suspension, and stained with various fluorophore-conjugated antibodies for immunophenotyping via flow cytometry.

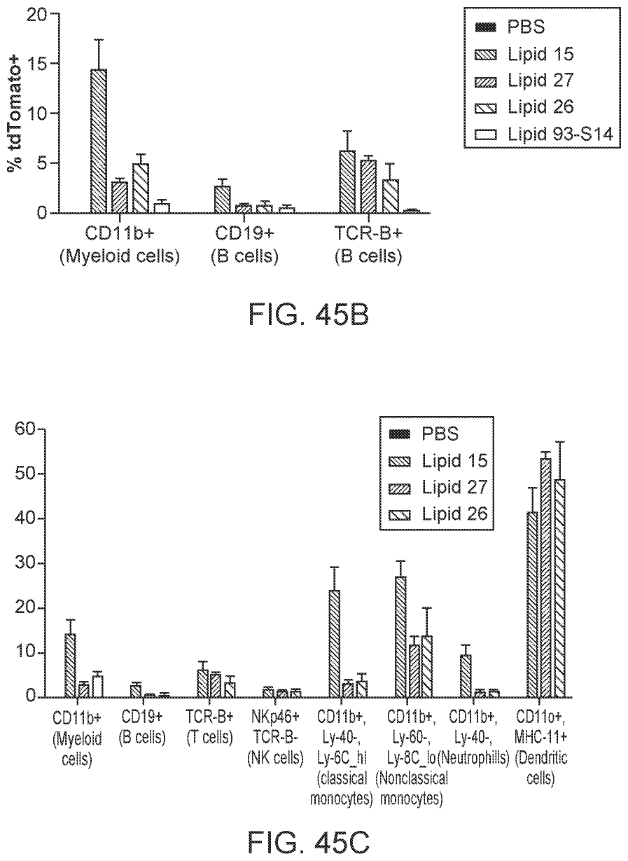

FIG. 45A shows representative FACS plots with frequencies of tdTomato expression in various spleen immune cell (CD45+, live) subsets, including total myeloid (CD11b+), B cells (CD19+), and T cells (TCR-B+) following treatment with LNPs formed with Lipid 10a-27 or 10a-26 or Lipid 10b-15. Ai9 mice injected with PBS represented background tdTomato fluorescence. FIG. 45B quantifies the proportion of myeloid cells, B cells, and T cells expressing tdTomato (mean+std. dev., n=3), which is equivalent to the proportion of each cell population which has been successfully transfected with Cre circular RNA. LNPs made with Lipids 10a-27 and 10a-26 exhibit significantly higher myeloid and T cell transfection compared with Lipid 93-S14, highlighting the improvements conferred by lipid structural modifications.

FIG. 45C illustrates the proportion of additional splenic immune cell populations expressing tdTomato with Lipids 10a-27 and 10a-26 (mean+std. dev., n=3), which also include NK cells (NKp46+, TCR-B−), classical monocytes (CD11b+, Ly-6G−, Ly-6C_hi), nonclassical monocytes (CD11b+, Ly-6G−, Ly-6C_lo), neutrophils (CD11b+, Ly-6G+), and dendritic cells (CD11c+, MHC-II+). These experiments demonstrate that LNPs made with Lipids 10a-27 and 10a-26 and Lipid 10b-15 are effective at delivering circular RNAs to many splenic immune cell subsets in mice and lead to successful protein expression from the circular RNA in those cells.

### Example 51

### Example 51A: Built-In polyA Sequences and Affinity-Purification to Produce Immune-Silent Circular RNA

PolyA sequences (20-30 nt) were inserted into the 5′ and 3′ ends of the RNA construct (precursor RNA with built-in polyA sequences in the introns). Precursor RNA and introns can alternatively be polyadenylated post-transcriptionally using, e.g., E. coli. polyA polymerase or yeast polyA polymerase, which requires the use of an additional enzyme.

Circular RNA in this example was circularized by in vitro transcription (IVT) and affinity-purified by washing over a commercially available oligo-dT resin to selectively remove polyA-tagged sequences (including free introns and precursor RNA) from the splicing reaction. The IVT was performed with a commercial IVT kit (New England Biolabs) or a customerized IVT mix (Orna Therapeutics), containing guanosine monophosphate (GMP) and guanosine triphosphate (GTP) at different ratios (GMP:GTP=8, 12.5, or 13.75). In some embodiments, GMP at a high GMP:GTP ratio may be preferentially included as the first nucleotide, yielding a majority of monophosphate-capped precursor RNAs. As a comparison, the circular RNA product was alternatively purified by the treatment with Xrn1, Rnase R, and Dnase I (enzyme purification).

Immunogenicity of the circular RNAs prepared using the affinity purification or enzyme purification process were then assessed. Briefly, the prepared circular RNAs were transfected into A549 cells. After 24 hours, the cells were lysed and interferon beta-1 induction relative to mock samples was measured by qPCR. 3p-hpRNA, a triphosphorylated RNA, was used as a positive control.

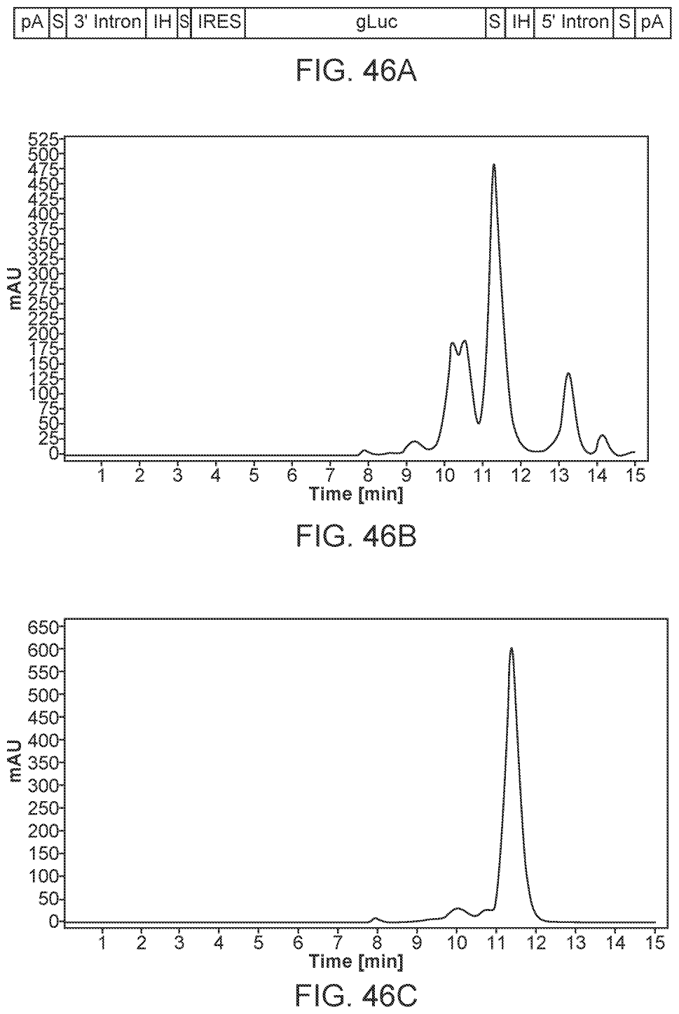

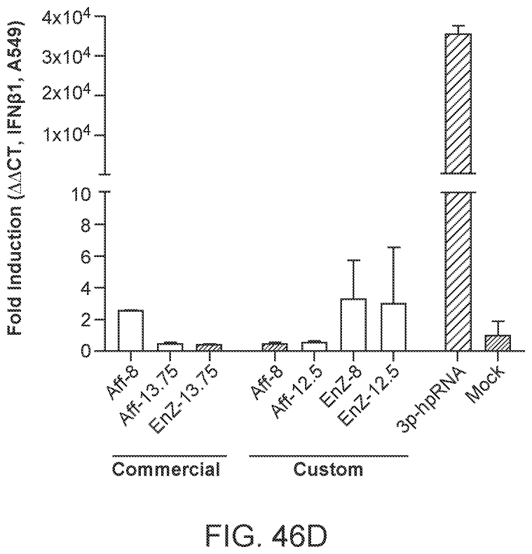

FIGS. 46B and 46C show that the negative selection affinity purification removes non-circular products from splicing reactions when polyA sequences are included on elements that are removed during splicing and present in unspliced precursor molecules. FIG. 46D shows circular RNAs prepared with tested IVT conditions and purification methods are all immunoquiescent. These results suggest the negative selection affinity purification is equivalent or superior to enzyme purification for circular RNA purification and that customized circular RNA synthesis conditions (IVT conditions) may reduce the reliance on GMP excess to achieve maximal immunoquiescence.

### Example 51B: Dedicated Binding Site and Affinity-Purification for Circular RNA Production

Instead of polyA tags, one can include specifically design sequences (DBS, dedicated binding site).

Instead of a polyA tag, a dedicated binding site (DBS), such as a specifically designed complementary oligonucleotide that can bind to a resin, may be used to selectively deplete precursor RNA and free introns. In this example, DBS sequences (30 nt) were inserted into the 5′ and 3′ ends of the precursor RNA. RNA was transcribed and the transcribed product was washed over a custom complementary oligonucleotide linked to a resin.

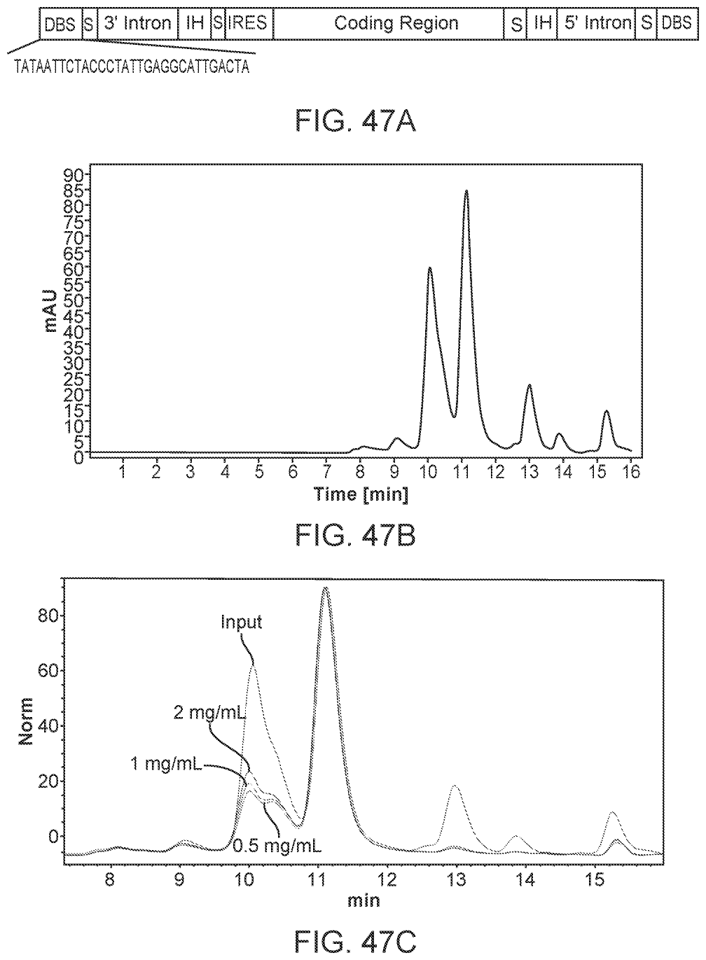

FIGS. 47B and 47C demonstrates that including the designed DBS sequence in elements that are removed during splicing enables the removal of unspliced precursor RNA and free intron components in a splicing reaction, via negative affinity purification.

### Example 51C: Production of a Circular RNA Encoding Dystrophin

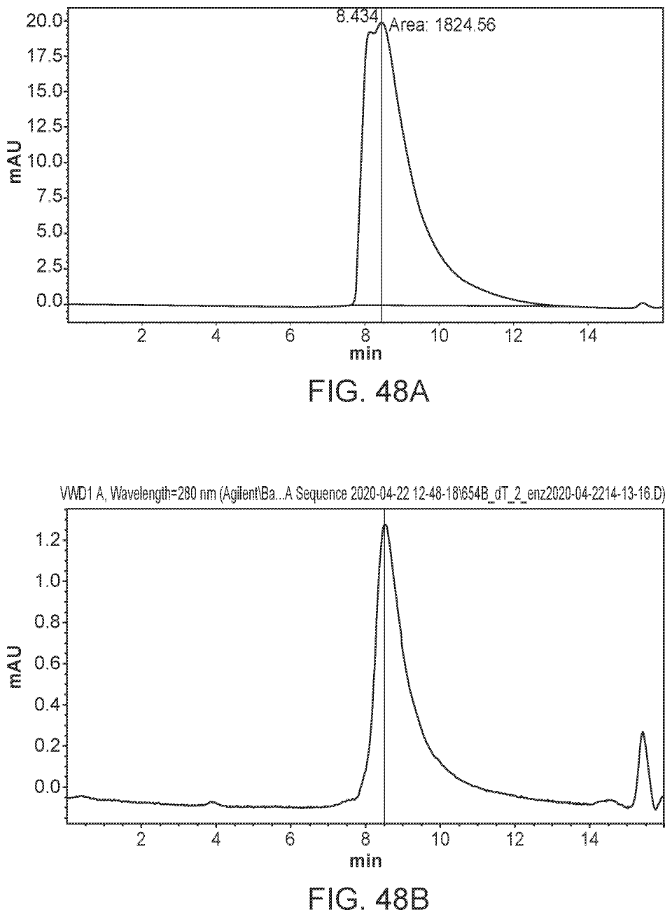

A 12 kb 12,000 nt circular RNA encoding dystrophin was produced by in vitro transcription of RNA precursors followed by enzyme purification using a mixture of Xrn1, DNase 1, and RNase R to degrade remaining linear components. FIG. 48 shows that the circular RNA encoding dystrophin was successfully produced.

### Example 52

### 5′ Spacer Between 3′ Intron Fragment and the IRES Improves Circular RNA Expression

Expression level of purified circRNAs with different 5′ spacers between the 3′ intron fragment and the IRES in Jurkat cells were compared. Briefly, luminescence from secreted Gaussia luciferase in supernatant was measured 24 hours after electroporation of 60,000 cells with 250ng of each RNA.

Additionally, stability of purified circRNAs with different 5′ spacers between the 3′ intron fragment and the IRES in Jurkat cells were compared. Briefly, luminescence from secreted Gaussia luciferase in supernatant was measured over 2 days after electroporation of 60,000 cells with 250ng of each RNA and normalized to day 1 expression.

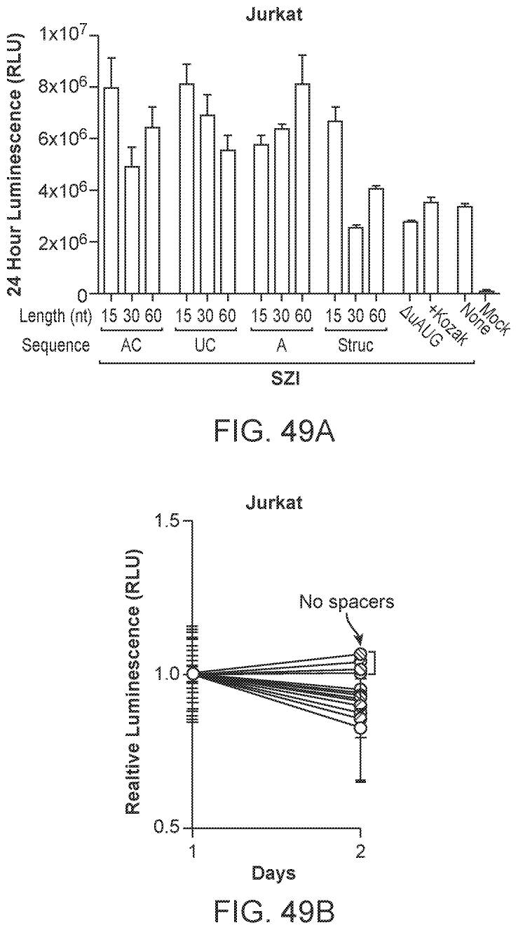

The results are shown in FIGS. 49A and 49B, indicating that adding a spacer can enhance LRES function and the importance of sequence identity and length of the added spacer. A potential explanation is that the spacer is added right before the IRES and likely functions by allowing the LRES to fold in isolation from other structured elements such as the intron fragments.

### Example 53

This example describes deletion scanning from 5′ or 3′ end of the caprine kobuvirus IRES. IRES borders are generally poorly characterized and require empirical analysis, and this example can be used for locating the core functional sequences required for driving translation. Briefly, circular RNA constructs were generated with truncated IRES elements operably linked to a gaussia luciferase coding sequence. The truncated IRES elements had nucleotide sequences of the indicated lengths removed from the 5′ or 3′ end. Luminescence from secreted gaussia luciferase in supernatant was measured 24 and 48 hours after electroporation of primary human T cells with RNA. Stability of expression was calculated as the ratio of the expression level at the 48-hour time point relative to that at the 24-hour time point.

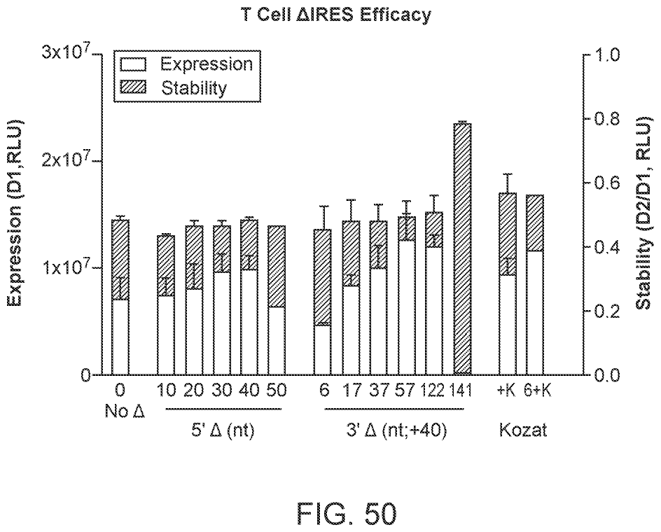

As shown in FIG. 50, deletion of more than 40 nucleotides from the 5′ end of the IRES reduced expression and disrupted IRES function. Stability of expression was relatively unaffected by the truncation of the IRES element but expression level was substantially reduced by deletion of 141 nucleotides from the 3′ end of the IRES, whereas deletion of 57 or 122 nucleotides from the 3′ end had a positive impact on the expression level.

It was also observed that deletion of the 6-nucleotide pre-start sequence reduced the expression level of the luciferase reporter. Replacement of the 6-nucleotide sequence with a classical kozak sequence (GCCACC) did not have a significant impact but at least maintained expression.

### Example 54

This example describes modifications (e.g., truncations) of selected selected IRES sequences, including Caprine Kobuvirus (CKV) IRES, Parabovirus IRES, Apodemus Picornavirus (AP) IRES, Kobuvirus SZAL6 IRES, Crohivirus B (CrVB) IRES, CVB3 IRES, and SAFV IRES. The sequences of the IRES elements are provided in SEQ ID NOs: 348-389. Briefly, circular RNA constructs were generated with truncated IRES elements operably linked to a gaussia luciferase coding sequence. HepG2 cells were transfected with the circular RNAs. Luminescence in the supernatant was assessed 24 and 48 hours after transfection. Stability of expression was calculated as the ratio of the expression level at the 48-hour time point relative to that at the 24-hour time point.

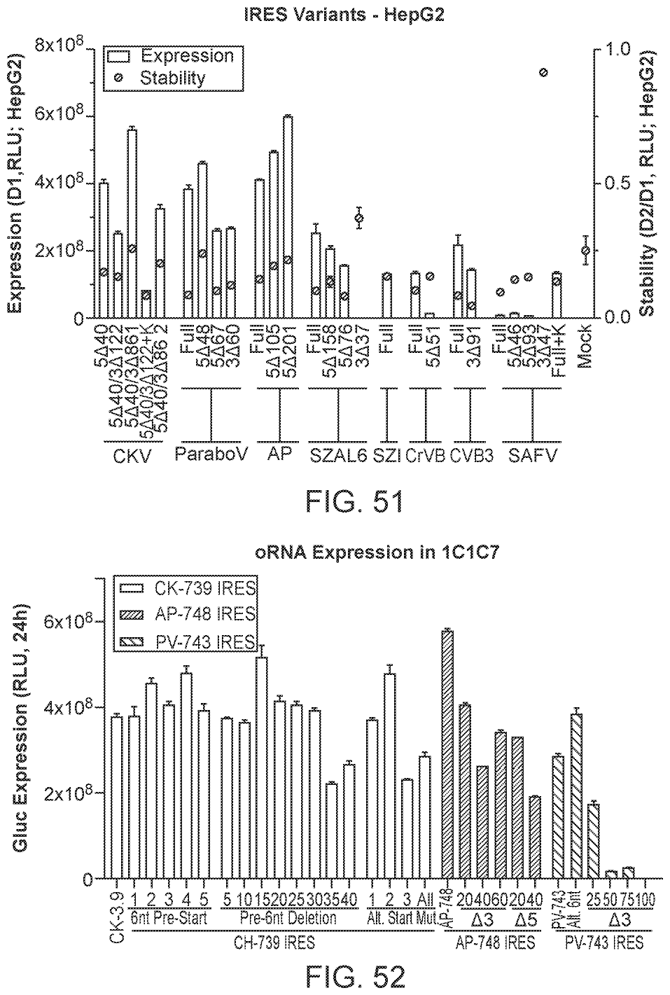

As shown in FIG. 51, truncations had variable effects depending on the identity of the IRES, which may depend on the initiation mechanism and protein factors used for translation, which often differs between IRESs. 5′ and 3′ deletions can be effectively combined, for example, in the context of CKV IRES. Addition of a canonical Kozak sequence in some cases significantly improved expression (as in SAFV, Full vs Full+K) or diminished expression (as in CKV, 5d40/3d122 vs 5d40/3d122+K).

### Example 55

This example describes modifications of CK-739, AP-748, and PV-743 IRES sequences, including mutations alterative translation initiation sites. Briefly, circular RNA constructs were generated with modified IRES elements operably linked to a gaussia luciferase coding sequence. Luminescence from secreted gaussia luciferase in supernatant was measured 24 and 48 hours after transfection of 1C1C7 cells with RNA.

CUG was the most commonly found alternative start site but many others were also characterized. These triplets can be present in the IRES scanning tract prior to the start codon and can affect translation of correct polypeptides. Four alternative start site mutations were created, with the IRES sequences provided in SEQ ID NOs: 378-380. As shown in FIG. 52, mutations of alternative translation initiation sites in the CK-739 IRES affected translation of correct polypeptides, positively in some instances and negatively in other instances. Mutation of all the alternative translation initiation sites reduced the level of translation.

Alternative Kozak sequences, 6 nucleotides before start codon, can also affect expression levels. The 6-nucleotide sequence upstream of the start codon were gTcacG, aaagtc, gTcacG, gtcatg, gcaaac, and acaacc, respectively, in CK-739 IRES and Sample Nos. 1-5 in the “6 nt Pre-Start” group. As shown in FIG. 52, substitution of certain 6-nucleotide sequences prior to the start codon affected translation.

It was also observed that 5′ and 3′ terminal deletions in AP-748 and PV-743 IRES sequences reduced expression. However, in the CK-739 IRES, which had a long scanning tract, translation was relatively unaffected by deletions in the scanning tract.

### Example 56

This example describes modifications of selected IRES sequences by inserting 5′ and/or 3′ untranslated regions (UTRs) and creating IRES hybrids. Briefly, circular RNA constructs were generated with modified IRES elements operably linked to a gaussia luciferase coding sequence. Luminescence from secreted gaussia luciferase in supernatant was measured 24 and 48 hours after transfection of HepG2 cells with RNA.

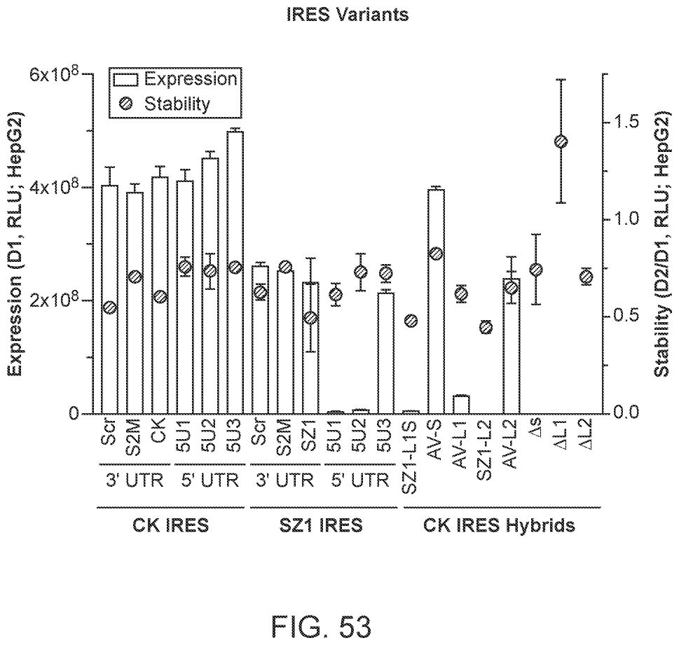

IRES sequences with UTRs inserted are provided in SEQ ID NOs: 390-401. As shown in FIG. 53, insertion of 5′ UTR right after the 3′ end of the IRES and before the start codon slightly increased the translation from Caprine Kobuvirus (CK) IRES but in some instances abrogated translation from Salivirus SZ1 IRES. Insertion of 3′ UTR right after the stop cassette had no impact on both IRES sequences.

Hybrid CK IRES sequences are provided in SEQ ID NOs: 390-401. CK IRES was used as a base, and specific regions of the CK IRES were replaced with similar-looking structures from other IRES sequences, for example, SZ1 and AV (Aichivirus). As shown in FIG. 53, certain hybrid synthetic IRES sequences were functional, indicating that hybrid IRES can be constructed using parts from distinct IRES sequences that show similar predicted structures while deleting these structures completely abrogates IRES function.

### Example 57

This example describes modifications of circular RNAs by introducing stop codon or cassette variants. Briefly, circular RNA constructs were generated with IRES elements operably linked to a gaussia luciferase coding sequence followed by variable stop codon cassettes, which included a stop codon in each frame and two stop codons in the reading frame of the gaussia luciferase coding sequence. 1C1C7 cells were transfected with the circular RNAs. Luminescence in supernatant was assessed 24 and 48 hours after transfection.

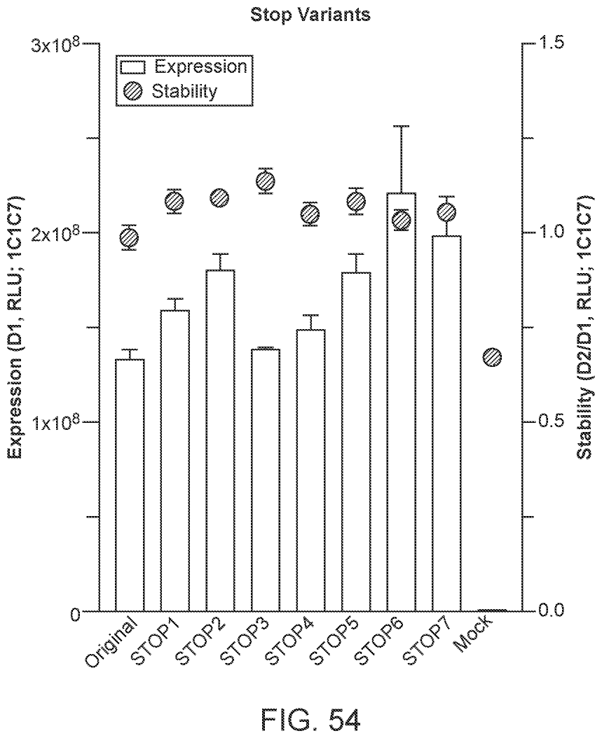

The sequences of the stop codon cassettes are set forth in SEQ ID NOs: 406-412. As shown in FIG. 54, certain stop codon cassettes improved expression levels, although they had little impact on expression stability. In particular, a stop cassette with two frame 1 (the reading frame of the gaussia luciferase coding sequence) stop codons, the first being TAA, followed by a frame 2 stop codon and a frame 3 stop codon, is effective for promoting functional translation.

### Example 58

This example describes modifications of circular RNAs by inserting 5′ UTR variants. Briefly, circular RNA constructs were generated with IRES elements with 5′ UTR variants inserted between the 3′ end of the IRES and the start codon, the IRES being operably linked to a gaussia luciferase coding sequence. 1C1C7 cells were transfected with the circular RNAs. Luminescence in supernatant was assessed 24 and 48 hours after transfection.

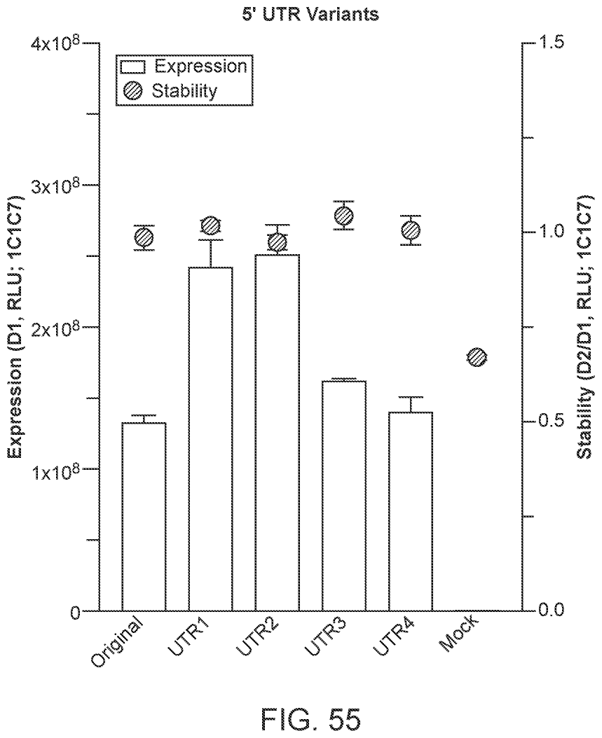

The sequences of the 5′ UTR variants are set forth in SEQ ID NOs: 402-405. As shown in FIG. 55, a CK IRES with a canonical Kozak sequence (UTR4) was more effective when a 36-nucleotide unstructured/low GC spacer sequence was added (UTR2), suggesting that the GC-rich Kozak sequences may interfere with core IRES folding. Using a higher-GC/structured spacer with a kozak sequence did not show the same benefit (UTR3), possibly due to interference with IRES folding by the spacer itself. Mutating the kozak sequence to gTcacG (UTR1) enhanced translation to the same level as the Kozak+spacer alternative without the need for a spacer.

### Example 59

This example describes the impact of miRNA target sites in circular RNAs on expression levels. Briefly, circular RNA constructs were generated with IRES elements operably linked to a human erythropoietin (hEPO) coding sequence, where 2 tandem miR-122 target sites were inserted into the construct. miR-122-expressing Huh7 cells were transfected with the circular RNAs. hEPO expression in supernatant was assessed 24 and 48 hours after transfection by sandwich ELISA.

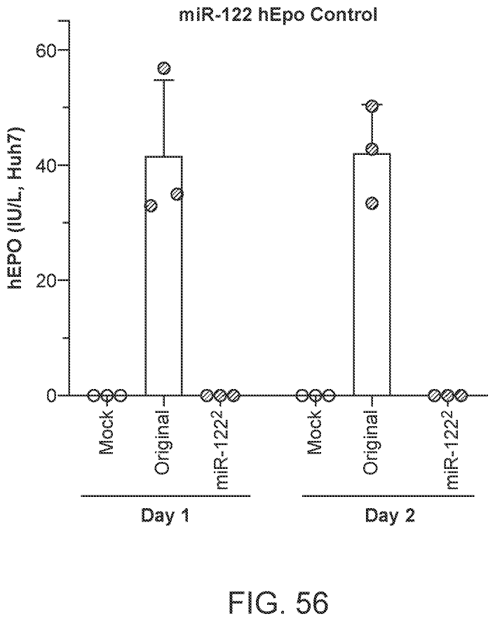

As shown in FIG. 56, the hEPO expression level was obrogated where the miR-122 target sites were inserted into the circular RNA. This result demonstrates that expression from circular RNA can be regulated by miRNA. As such, cell type- or tissue-specific expression can be achieved by incorporating target sites of the miRNAs expressed in the cell types in which expression of the recombinant protein is undesirable.

### Example 60

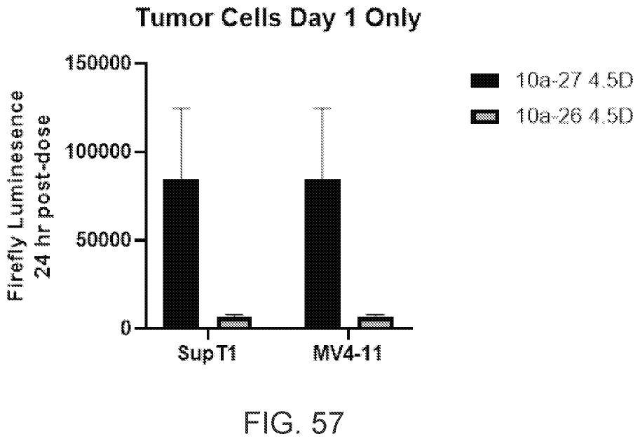

This example shows transfection of human tumor cells by LNPs in vitro. SupT1 cells (a human T cell tumor line) and MV4-11 cells (a human macrophage tumor line) were plated at 100,000 cells/well and 100,000 cells/well, respectively, in a 96-well plate overnight. Then, LNPs containing circRNA coding for Firefly Luciferase (FLuc) were added to the cells at 200 ng RNA/well. After 24-hour incubation, luminescence was quantified using the Bright-Glo Luciferase Assay System (Promega) according to manufacturer's instructions and background luminescence from cells not treated with LNP was subtracted. FIG. 57 quantifies the measured Firefly luminescence, indicating that LNPs comprising Lipid 10a-27 (10a-27 (4.5D) LNP, see Example 70) or Lipid 10a-26 (10a-26 (4.5D) LNP, see Example 70) can transfect and express circRNA in both human T cell and macrophage tumor lines in vitro. 10a-27 (4.5D) LNP resulted in higher luminescence than 10a-26 (4.5D) LNP showing that levels of transfection of LNPs to human tumor cells can be affected by formulation.

### Example 61

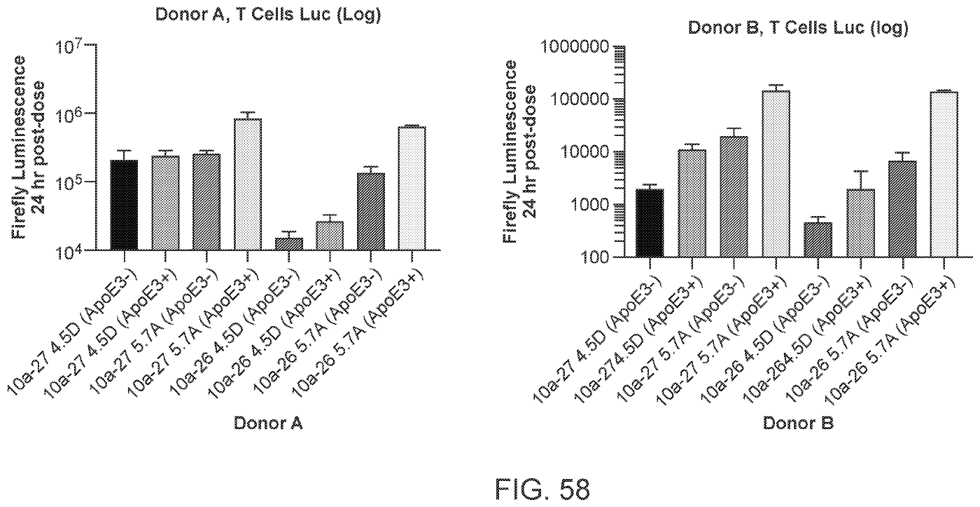

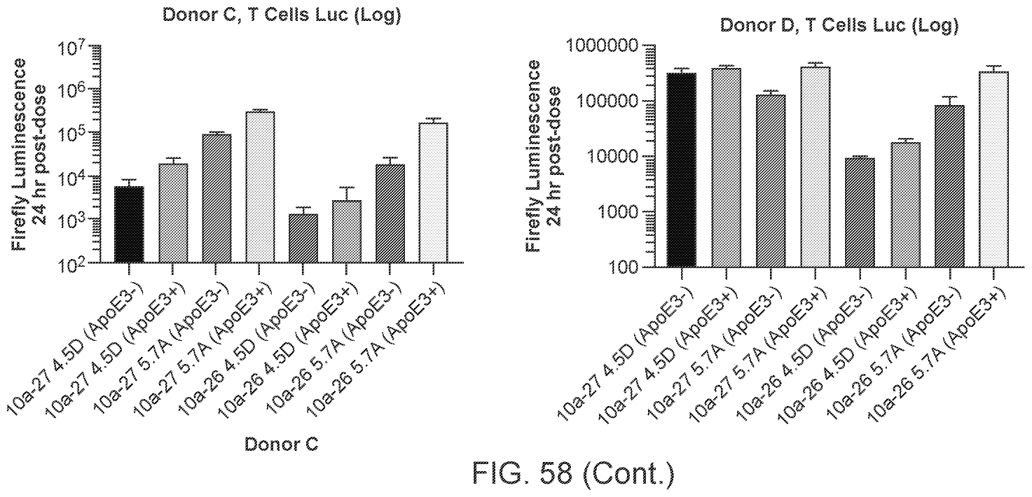

This example shows transfection of primary human activated T cells in vitro. Primary human T cells from independent donors were stimulated with aCD3/aCD28 and allowed to proliferate for 6 days in the presence of human serum and IL-2. Then, 100,000 cells were plated in a 96 well plate and LNPs containing circRNA coding for Firefly Luciferase (FLuc) were added to the cells at 200 ng RNA/well with or without Apolipoprotein E3 (ApoE3). After 24-hour incubation, luminescence was quantified using the Bright-Glo Luciferase Assay System (Promega) according to manufacturer's instructions and background luminescence from cells not treated with LNPs was subtracted. FIG. 58 shows the measured Firefly luminescence across 4 independent donors, demonstrating that all LNPs tested transfect primary human T cells in vitro. LNPs containing Lipid 10a-27 generally produced higher luminescence than those containing Lipid 10a-26. Furthermore, the addition of ApoE3 generally increased the expression of luciferase more for 10a-27 (5.7A) and 10a-26 (5.7A) (average of 4.4-fold and 9.3-fold across 4 donors, respectively) compared to 10a-27 (4.5D) and 10a-26 (4.5D) (3.1-fold and 2.6-fold, respectively). This suggests that the helper lipid, PEG lipid, and ionizable lipid:phosphate ratio all contribute to the ApoE-dependence of different formulations made with the same ionizable lipids. (See Example 70 for LNP formulation procesure, e.g., for 10a-27 (5.7A), 10a-26 (5.7A), 10a-27 (4.5D), and 10a-26 (4.5D) LNPs.)

### Example 62

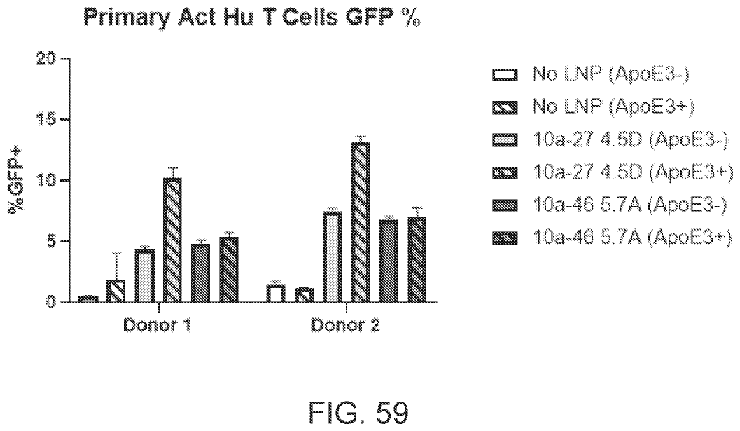

This example shows that different tail chemistries of LNPs result in different uptake mechanisms into T cells. To quantify the percent of human T cells expressing circRNA, LNPs containing eGFP circRNA were added to activated primary human T cells (prepared as described above in Example 61) at 200 ng RNA/well with or without ApoE3. After 24-hour incubation, cells were analyzed by flow cytometry and the percentage of live, GFP+ T cells was quantified. FIG. 59 graphs the % GFP+ T cells for 2 independent donors, with 5-10% of cells being GFP+ for LNP containing Lipid 10a-27 (10a-27 (4.5D) LNP, see Example 70) and for LNP contacting Lipid 10a-46 (10a-46 (5.7A) LNP, see Example 70). Although ApoE3 addition resulted in increased transfection for 10a-27 (4.5D) LNP, it did not appear to increase transfection for 10a-46 (5.7A) LNP, suggesting the different tail chemistries between Lipids 10a-27 and 10a-46 may mediate different uptake mechanisms into T cells.

### Example 63

This example describes immune cell expression of Cre in a Cre reporter mouse model.

Ai9 mice (B6.Cg-Gt(ROSA)26Sortm9(CAG-tdTomato)Hze/J, female, 6-8 weeks, n=3 per group) were injected i.v. with 0.5 mg/kg Cre circRNA LNPs or PBS. Ai9 mice transcribe and translate the fluorescent reporter tdTomato upon Cre recombination; meaning cells which are tdTomato+ have successfully been transfected with Cre circRNA. After 48 hours, mice were euthanized and their spleens were collected and manually processed into single cell suspensions. The splenocytes were stained for dead cells (LiveDead Fixable Aqua, Thermo) and with anti-mouse antibodies (TCR-B chain, BV421, H57-597; CD45, BV711, 30-F11; CD11b, BV785, ICRF44; NKp46, AF647, 29A1.4; CD19, APC/750, 6D5; TruStain FcX, 93; all antibodies from Biolegend) at 1:200 ratio. Flow cytometry was performed using an Attune Nxt Flow Cytometer (Thermo).

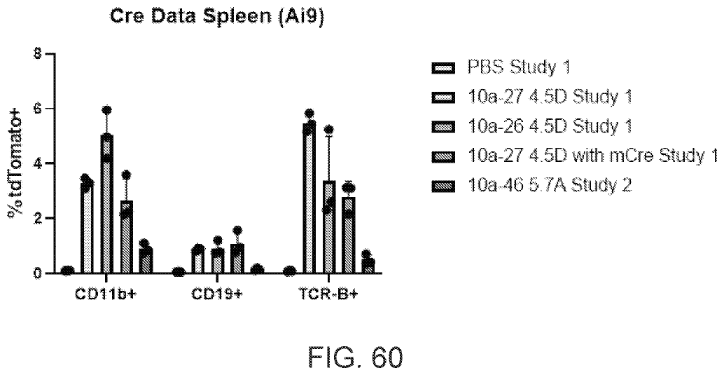

The percent of tdTomato+ cells in splenic myeloid cells (CD11b+), B cells (CD19+), and T cells (TCR-B+) is presented in FIG. 60. Lipid 10a-27 and Lipid 10a-46 differ only by their tail chemistries, and formulations made with Lipid 10a-27 transfect significantly more splenic immune cells than those made with Lipid 10a-46. Additionally, 10a-27 (4.5D) LNP (see Example 70) formulated with Cre circRNA transfected approximately twice as many T cells than those formulated with Cre linear mRNA, suggesting that circRNA may result in improved protein expression in splenic T cells compared to linear mRNA.

TABLE 17
Characterization of LNPs

| | | | RNA Encapsulation |
|---|---|---|---|
| Formulation | Z-Average (nm) | PDI | Efficiency (%) |
| 10a-27 (4.5D), Study 1 | 65 | 0.07 | 96 |
| 10a-26 (4.SD), Study 1 | 74 | 0.06 | 94 |
| 10a-27 (4.5D), with mCre, Study 1 | 75 | 0.05 | 93 |
| 10a-46 (5.7A), Study 2 | 86 | 0.01 | 94 |

### Example 64

This example shows immune cell expression of mOX40L circRNA in wildtype mice.

C57BL/6 mice (female, 6-8 weeks, n=3 or 4 per group) were injected intravenously with 0.5 mg/kg mOX40L circRNA LNPs or PBS. After 24 hours, mice were euthanized and their spleens were collected and manually processed into single cell suspensions. Splenocytes were stained for dead cells (LiveDead Fixable Aqua, Thermo) and with anti-mouse antibodies (TCR-B chain, PacBlue, H57-597; CD11b, FITC, ICRF44; B220, PE, RA3-6B2; CD45, PerCP, 30-F11; mOX40L, AF647, RM134L; NK1.1, APC/750, PK136; TruStain FcX, 93; all antibodies from Biolegend) at 1:200. Flow cytometry was performed using an Attune NxT Flow Cytometer (Thermo).

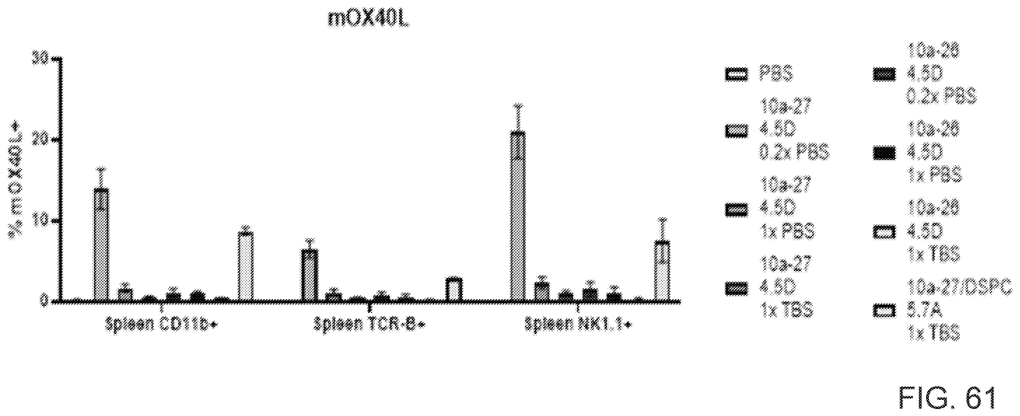

The percent of mOX40L+ cells in splenic myeloid (CD11b+), T cells (TCR-B+), and NK cells (NK1.1+) is presented in FIG. 61. Notably, significantly different transfection efficiencies are observed between the same formulations injected intravenously in different buffers (hypotonic PBS, isotonic PBS, and isotonic TBS). 10a-27 4.5D LNP in hypotonic PBS results in approximately 14% myeloid cell transfection, 6% T cell transfection, and 21% NK cell transfection in the spleen. Of the formulations injected in isotonic buffer, 10a-27 DSPC 5.7A LNP demonstrates myeloid, T cell, and NK cell transfection in the spleen (9%, 3%, and 8%, respectively). (See Example 70 for LNP formulation procesure, e.g., for 10a-27 (4.5D) LNP and 10a-27 DSPC (5.7A) LNP.)

TABLE 18
Characterization of LNPs

| | Z-Average | | RNA Encapsulation |
|---|---|---|---|
| Formulation | (nm) | PDI | Efficiency (%) |
| 10a-27 (4.5D) | 63 | 0.02 | 93 |
| 10a-26 (4.5D) | 67 | 0.07 | 94 |
| 10a-27 DSPC (5.7A) | 82 | 0.05 | 96 |

### Example 65

This example shows single dose escalation of mOX40L circRNA-LNPs in wildtype mice.

57BL/6 mice (female, 6-8 weeks, n=3 per group) were injected intravenously with 1 mg/kg or 3 mg/kg mOX40L circRNA LNPs or buffer control. After 24 hours, mice were euthanized and their spleens were collected and manually processed into single cell suspensions. Splenocytes were stained for dead cells (LiveDead Fixable Blue, Thermo) and stained with anti-mouse antibodies (TCR-B chain, BV421, H57-597; CD19, BV605, 6D5; CD45, BV711, 30-F11, CD11b, BV785, ICRF44; CD11c, FITC, N418; CD8a, PerCP, 53-6.7; mOX40L, PE, RM134L; NKp46, AF647, 29A1.4; CD4, APC/750, GK1.5; TruStain FcX, 93; all antibodies from Biolegend) at 1:200. Flow cytometry was performed using a BD FACSSymphony flow cytometer.

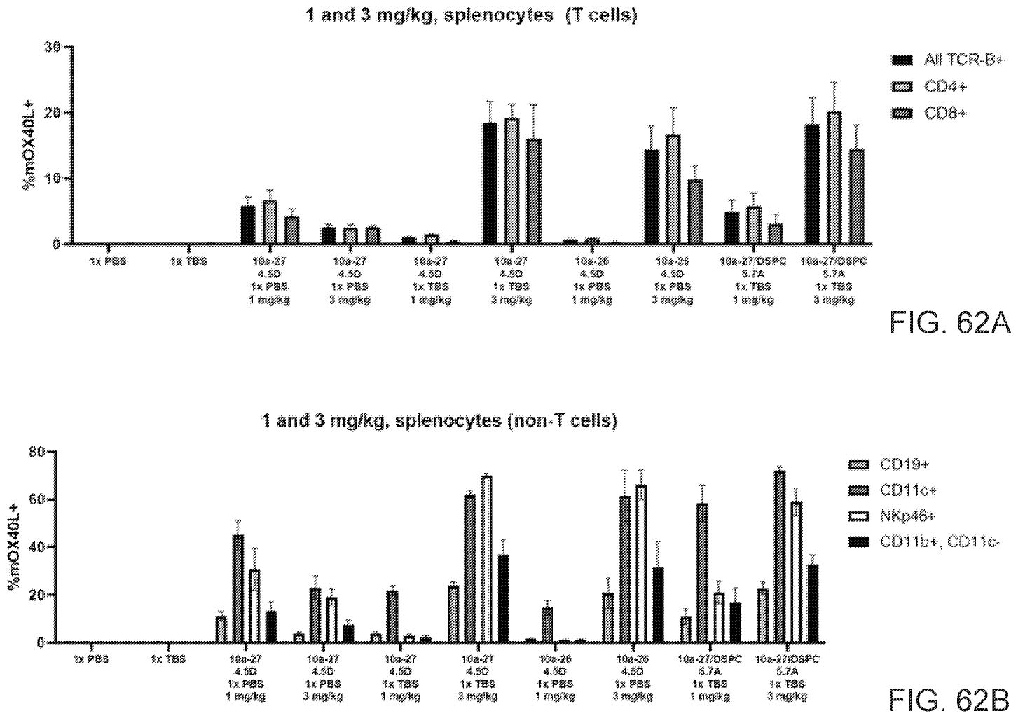

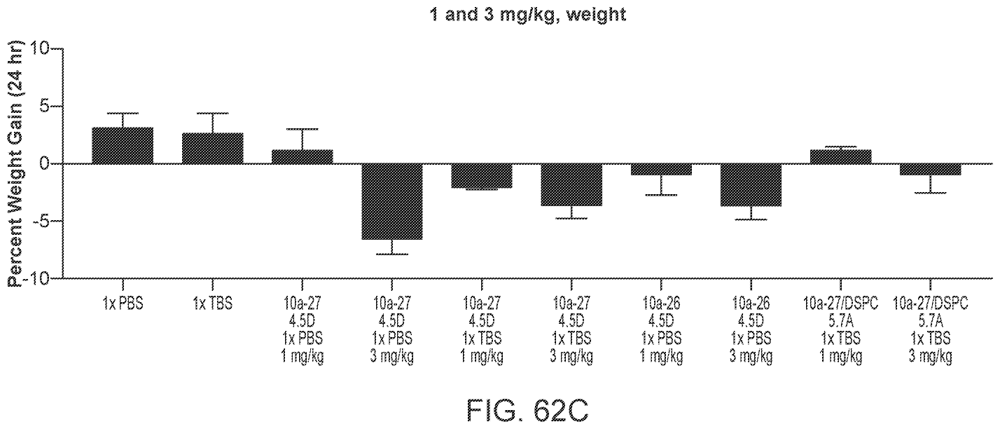

The percent of mOX40L+ cells in splenic T cells (all TCR-B+), CD4+ T cells (TCR-B+, CD4+), CD8+ T cells (TCR-B+, CD8a+), B cells (CD19+), NK cells (NKp46+), dendritic cells (CD11c+), and other myeloid cells (CD11b+, CD11c−) are shown in FIG. 62A and FIG. 62B, with corresponding mouse weight change after 24 hours shown in FIG. 62C. A dose-dependent increase in immune cell subset transfection is observed across 1 mg/kg and 3 mg/kg for all groups, with the exception of 10a-27 (4.5D) LNP 1×PBS group. At the 3 mg/kg dose, three different LNPs (10a-27 (4.5D) in TBS, 10a-26 (4.5D) in PBS, and 10a-27 DSPC (5.7A) in TBS; see Example 70 for formulation procedures) achieve 10-20% mOX40L transfection in splenic T cells, with similar transfection rates observed among CD4+ and CD8+ subsets. These three formulations also result in approximately 20% B cell, 60-70% dendritic cell, 60-70% NK cell, and 30-40% other myeloid cell mOX40L transfection in the spleen at 3 mg/kg. These three formulations lead to only minor (0-3%) mouse weight loss at 24 hours at the 3 mg/kg single dose with no reported clinical observations.

TABLE 19
Characterization of LNPs

| | Z-Average | | RNA Encapsulation |
|---|---|---|---|
| Formulation | (nm) | PDI | Efficiency (%) |
| 10a-27 (4.5D) | 76 | 0.06 | 91 |
| 10a-26 (4.5D) | 67 | 0.01 | 88 |
| 10a-27 DSPC (5.7A) | 77 | 0.01 | 93 |

### Example 66

This example shows circRNA-LNP CAR-mediated B cell depletion in mice.

C57BL/6 mice (female, 6-8 weeks, n=5 per group) were injected intravenously with 0.5 mg/kg aCD19-CAR circRNA LNPs or control FLuc circRNA LNPs on Days 0, 2, 5, 7, and 9. On Days −1, 1, 8 and 12, submandibular bleeds were performed to collect blood. 30 uL of blood was lysed with ACK lysis buffer and washed with MACS buffer to isolate immune cells. On Day 12, mice were euthanized and their spleens were collected and manually processed into single cell suspensions. To assess the frequency of B cells in the blood and spleen, these single cell suspensions were stained for dead cells (LiveDead Fixable Aqua, Thermo) and stained with anti-mouse antibodies (TCR-B chain, PacBlue, H57-597; CD11b, FITC, ICRF44; B220, PE, RA3-6B2; CD45, PerCP, 30-F11; TruStain FcX, 93; all antibodies from Biolegend) at 1.200. Flow cytometry was performed using an Attune NxT Flow Cytometer (Thermo).

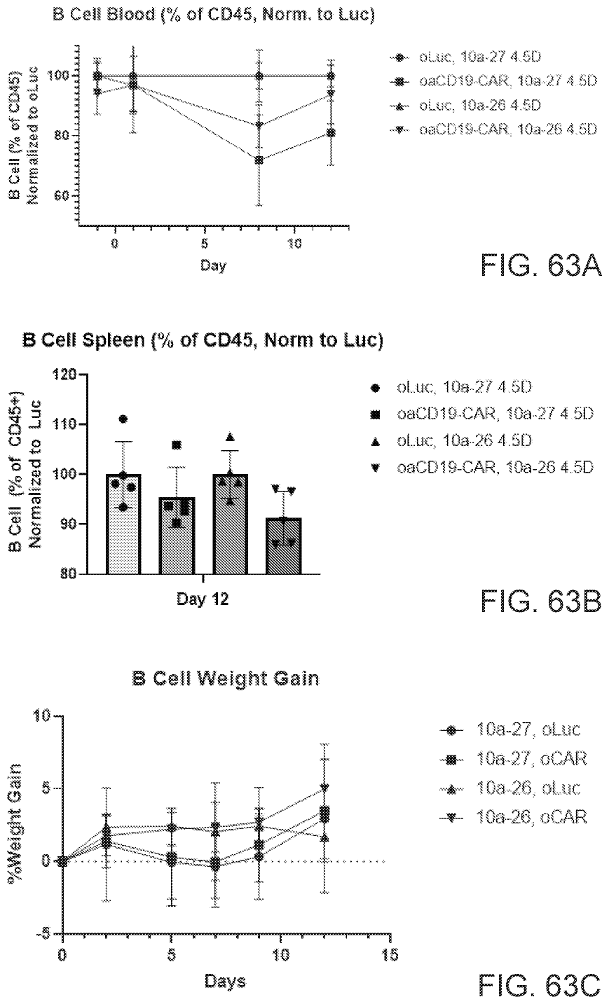

FIG. 63A quantifies the B cell depletion observed in this study, as defined by percentage of B220+ B cells of live, CD45+ immune cells. The B cell depletion in the aCD19-CAR circRNA LNP group was compared to its respective FLuc circRNA LNP control on Days 8 and 12 (for blood) and Day 12 (for spleen). In the blood, aCD19-CAR 10a-27 (4.5D) and 10a-26 (4.5D) LNPs resulted in approximately 28% and 17% reductions, respectively, in % B220+ of live CD45+ at Day 8 compared to FLuc control. In the spleen, aCD19-CAR 10a-27 (4.5D) and 10a-26 (4.5D) LNPs resulted in approximately 5% and 9% reductions in % B220+ of live CD45+ at Day 12 compared to FLuc control as shown in FIG. 63B. In all, these results suggest that CAR-mediated B cell depletion is occurring in mice treated with aCD19-CAR circRNA LNPs for Lipid 10a-27 (4.5D) and Lipid 10a-26 (4.5D).

In addition, FIG. 63C shows the percent weight gain of mice in this study. There was not significant weight loss on average from the 10a-27 4.5D or 10a-26 4.5D LNP treated mice (5×0.5 mg/kg over 9 days), suggesting that these LNPs may be well-tolerated in mice at this dose and schedule.

TABLE 20
Characterization of LNPs

| | Z-Average | | RNA Encapsulation |
|---|---|---|---|
| Formulation | (nm) | PDI | Efficiency (%) |
| 10a-27 (4.5D), oLuc | 65 | 0.03 | 93 |
| 10a-27 (4.5D), oaCD19-CAR | 74 | 0.02 | 96 |
| 10a-26 (4.5D), oLuc | 71 | 0.04 | 91 |
| 10a-26, (4.5D), oaCD19-CAR | 71 | 0.04 | 93 |

### Example 67

### LNP and Circular RNA Construct Containing Anti-CD19 CAR Reduces B Cells in the Blood and Spleen In Vivo.

Circular RNA constructs encoding an anti-CD19 CAR expression were encapsulated within lipid nanoparticles as described above. For comparison, circular RNAs encoding luciferase expression were encapsulated within separate lipid nanoparticle.

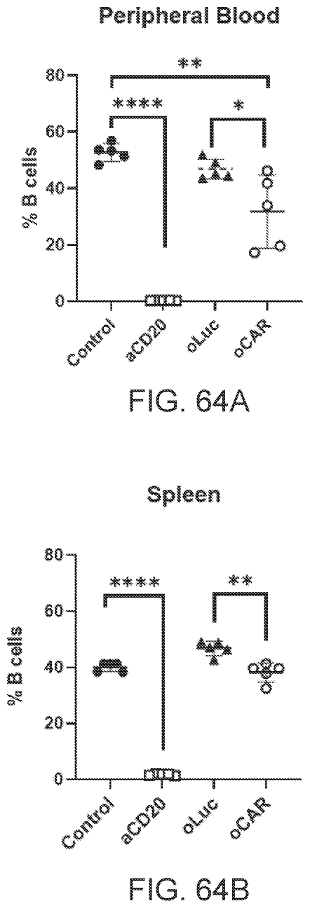

C57BL/6 mice at 6 to 8 weeks old were injected with either LNP solution every other day for a total of 4 LNP injections within each mouse. 24 hours after the last LNP injection, the mice's spleen and blood were harvested, stained, and analyzed via flow cytometry. As shown in FIG. 64A and FIG. 64B, mice containing LNP-circular RNA constructs encoding an anti-CD19 CAR led to a statistically significant reduction in CD19+ B cells in the peripheral blood and spleen compared to mice treated with LNP-circular RNA encoding a luciferase.

### Example 68

IRES Sequences Contained within Circular RNA Encoding CARs Improves CAR Expressions and Cytotoxicity of T-Cells.

Activated murine T-cells were electroporated with 200ng of circular RNA constructs containing a unique IRES and a murine anti-CD19 1D3ζ CAR expression sequence. The LRES contained in these constructs were derived either in whole or in part from a Caprine Kobuvirus, Apodemus Picornavirus, Parabovirus, or Salivirus. A Caprine Kobuvirus derived IRES was additionally codon optimized. As a control, a circular RNA containing a wild-type zeta mouse CAR with no IRES was used for comparison. The T-cells were stained for the CD-19 CAR 24 hours post electroporation to evaluate for surface expression and then co-cultured with A20 Fluc target cells. The assay was then evaluated for cytotoxic killing of the Fluc+A20 cells 24 hours after co-culture of the T-cells with the target cells.

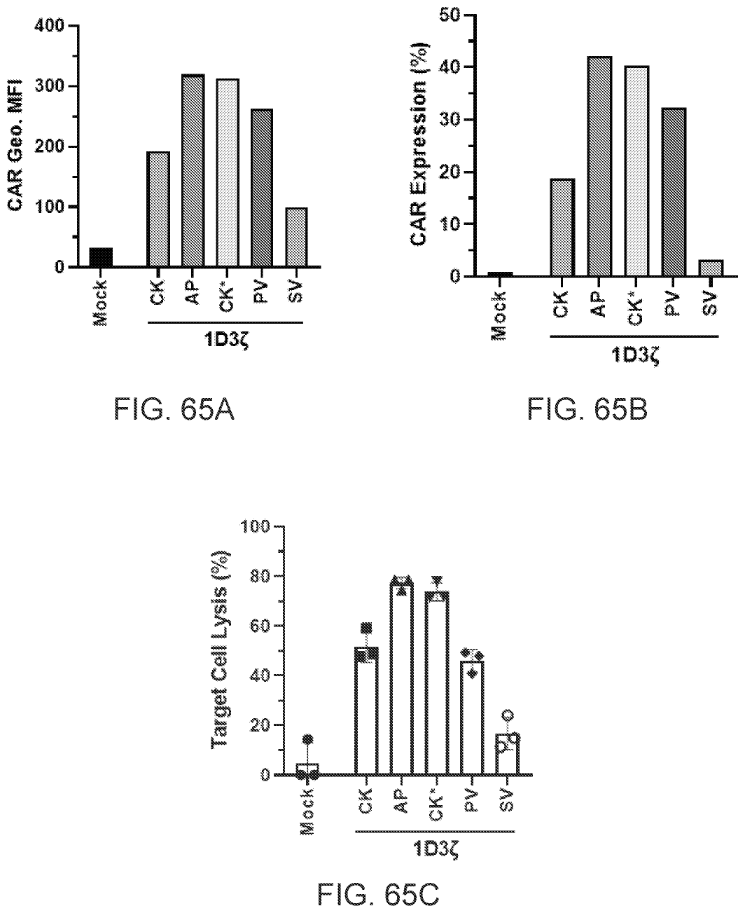

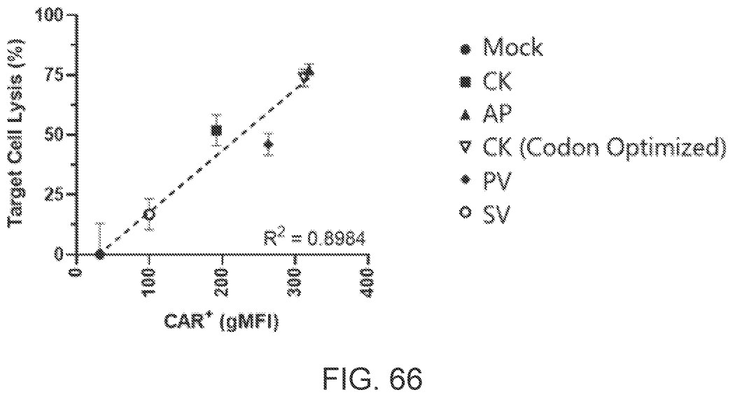

As seen in FIGS. 65A, 65B, 65C, and 66, the unique IRES were able to increase the frequency that the T-cells expressed the CAR protein and level of CAR expression on the surface of the cells. The increase frequency of expression of the CAR protein and level of CAR expression on the surface of cells lead to an improved anti-tumor response.

### Example 69

Cytosolic and Surface Proteins Expressed from Circular RNA Construct in Primary Human T-Cells.

Circular RNA construct contained either a sequence encoding for a fluorescent cytosolic reporter or a surface antigen reporter. Fluorescent reporters included green fluorescent protein, mCitrine, mWasabi, Tsapphire. Surface reporters included CD52 and Thy1.1^{bio}. Primary human T-cells were activated with an anti-CD3/anti-CD28 antibody and electroporated 6 days post activation of the circular RNA containing a reporter sequence. T-cells were harvested and analyzed via flow cytometry 24 hours post electroporation. Surface antigens were stained with commercially available antibodies (e.g., Biolegend, Miltenyi, and BD).

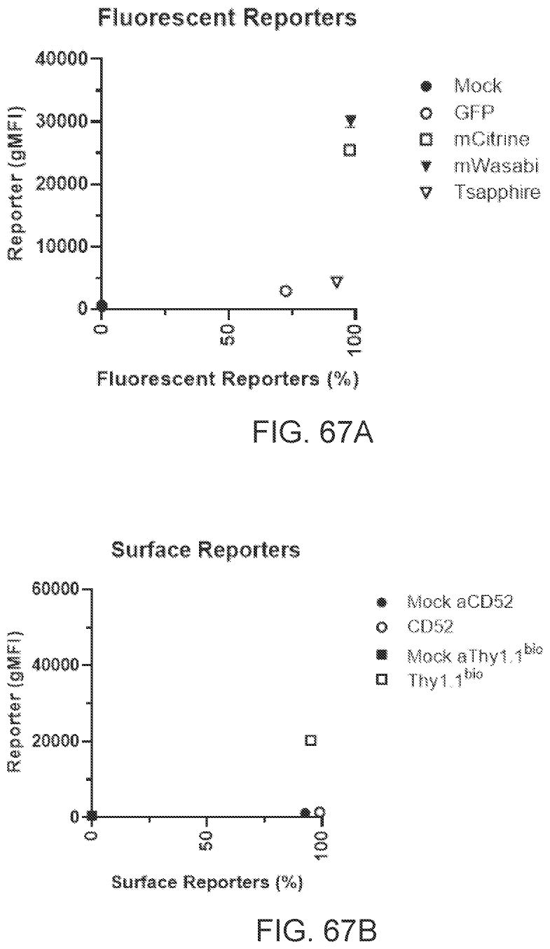

As seen in FIG. 67A and FIG. 67B, cytosolic and surface proteins can be expressed from circular RNA encoding the proteins in primary human T-cells.

### Example 70

Circular RNAs Containing Unique IRES Sequences have Improved Translation Expression Over Linear mRNA.

Circular RNA constructs contained a unique IRES along with an expression sequence for Firefly luciferase (FLuc).

Human T-cells from 2 donors were enriched and stimulated with anti-CD3/anti-CD28 antibodies. After several days of proliferation, activated T cells were harvested and electroporated with equal molar of either mRNA or circular RNA expressing FLuc payloads. Various IRES sequences, including those derived from Caprine Kobuvirus, Apodemus Picornavirus, and Parabovirus, were studied to evaluate expression level and durability of the payload expression across 7 days. Across the 7 days, the T-cells were lysed with Promega Brightglo to evaluate for bioluminsences.

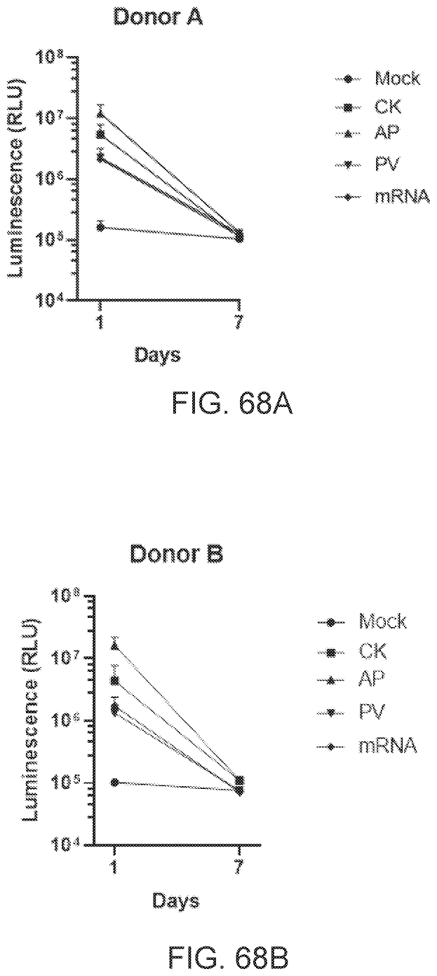

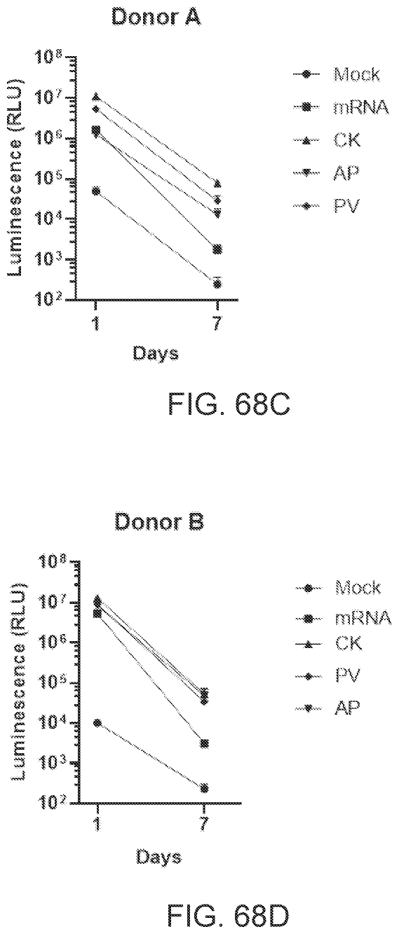

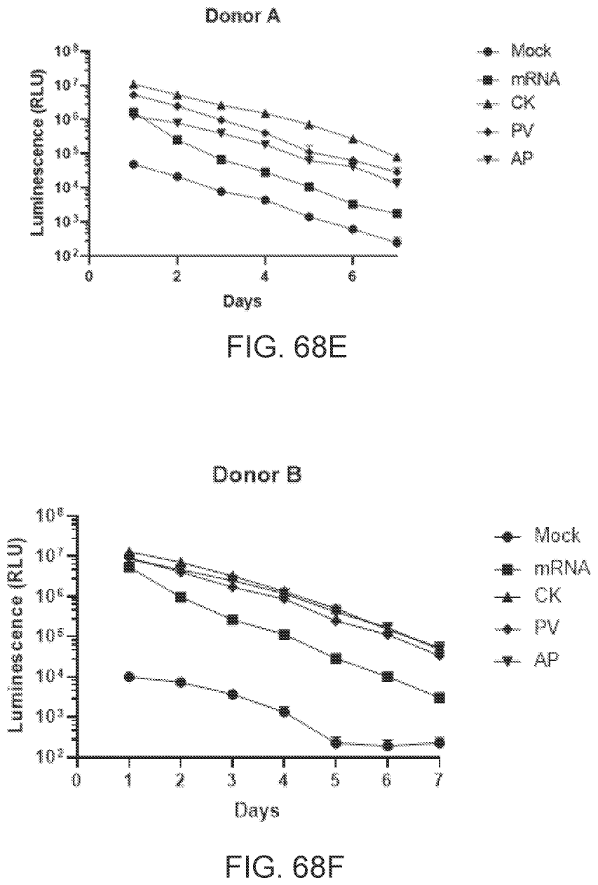

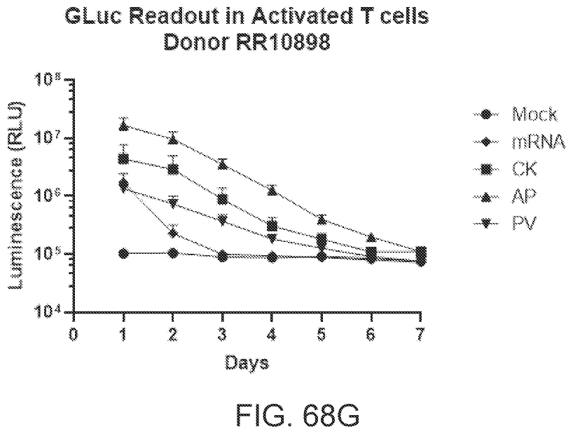

As shown in FIGS. 68C, 68D, 68E, 68F, and 68G, the presence of an IRES within a circular RNA can increase translation and expression of a cytosolic protein by orders of magnitude and can improve expression compared to linear mRNA. This was found consistent across multiple human T-cell donors.

### Example 71

### Example 71A: LNP-Circular RNA Encoding Anti-CD I19 Mediates Human T-Cell Killing of K562 Cells

Circular RNA constructs contained a sequence encoding for anti-CD19 antibodies. Circular RNA constructs were then encapsulated within a lipid nanoparticle (LNP).

Human T-cells were stimulated with anti-CD3/anti-CD28 and left to proliferate up to 6 says. At day 6, LNP-circular RNA and ApoE3 (1 μg/mL) were co-cultured with the T-cells to mediate transfection. 24 hours later, Fluc+K562 cells were electroporated with 200ng of circular RNA encoding anti-CD19 antibodies and were later co-cultured at day 7. 48 hours post co-culture, the assay was assessed for CAR expression and cytotoxic killing of K562 cells through Fluc expression.

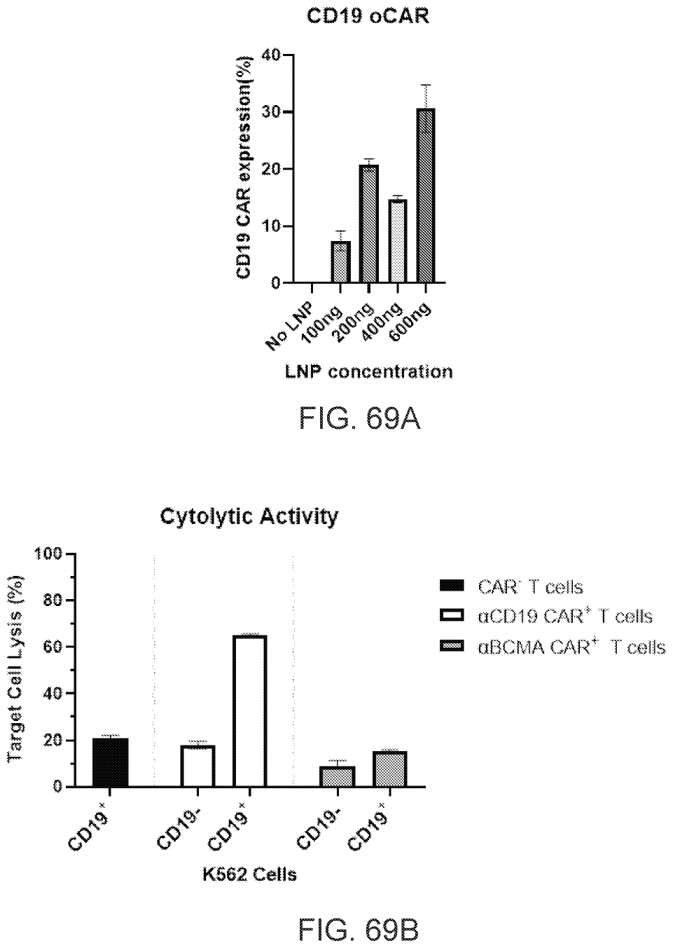

As shown in FIG. 69A and FIG. 69B, there is T-cell expression of anti-CD19 CAR from the LNP-mediated delivery of a CAR in vitro to T-cells and its capability to lyse tumor cells in a specific, antigen dependent manner in engineered K562 cells.

### Example 71B: LNP-Circular RNA Encoding Anti-BCMA Antibody Mediates Human T-Cell Killing of K562 Cells

Circular RNA constructs contained a sequence encoding for anti-BCMA antibodies. Circular RNA constructs were then encapsulated within a lipid nanoparticle (LNP).

Human T-cells were stimulated with anti-CD3/anti-CD28 and left to proliferate up to 6 says. At day 6, LNP-circular RNA and ApoE3 (1 μg/mL) were co-cultured with the T-cells to mediate transfection. 24 hours later, Fluc+K562 cells were electroporated with 200ng of circular RNA encoding anti-BCMA antibodies and were later co-cultured at day 7. 48 hours post co-culture, the assay was assessed for CAR expression and cytotoxic killing of K562 cells through Fluc expression.

As shown in FIG. 69B, there is T-cell expression of BCMA CAR from the LNP-mediated delivery of a CAR in vitro to T-cells and its capability to lyse tumor cells in a specific, antigen dependent manner in engineered K562 cells.

### Example 72

### Anti-CD19 CAR T-Cells Exhibit Anti-Tumor Activity In Vitro.

Human T-cells were activated with anti-CD3/anti-CD28 and electroporated once with 200ng of anti-CD19 CAR-expressing circular RNA. Electroporated T-cells were co-cultured with FLuc+ Nalm6 target cells and non-target Fluc+K562 cells to evaluate CAR-mediated killing. After 24 hours post co-culture, the T-cells were lysed and examined for remanent FLuc expression by target and non-target cells to evaluate expression and stability of expression across 8 days total.

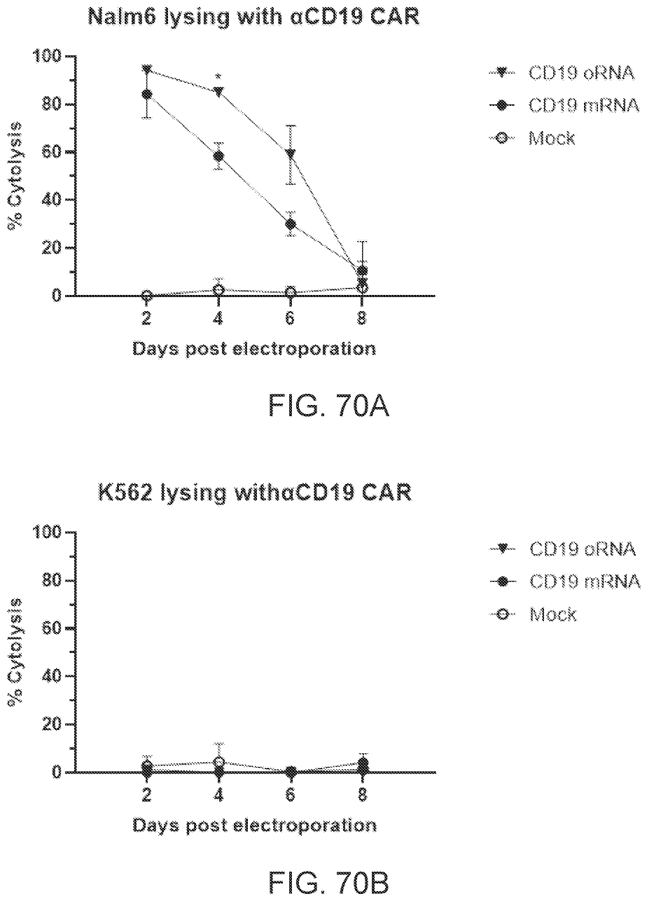

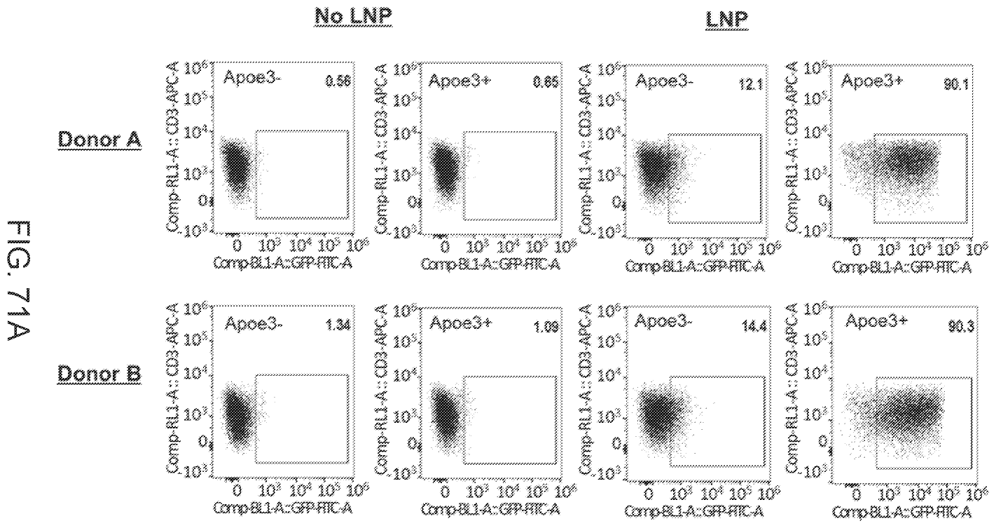

As shown in FIGS. 70A and 70B, T-cells express circular RNA CAR constructs in specific, antigen-dependent manner. Results also shows improved cytotoxicity of circular RNAs encoding CARs compared to linear mRNA encoding CARs and delivery of a functional surface receptor.

### Example 73

Effective LNP Transfection of Circular RNA Mediated with ApoE3

Human T-cells were stimulated with anti-CD3/anti-CD28 and left to proliferate up to 6 days. At day 6, lipid nanoparticle (LNP) was and circular RNA expressing green fluorescence protein solution with or without ApoE3 (1 μg/mL) were co-cultured with the T-cells. 24 hours later, the T-cells were stained for live/dead T-cells and the live T-cells were analyzed for GFP expression on a flow cytometer.

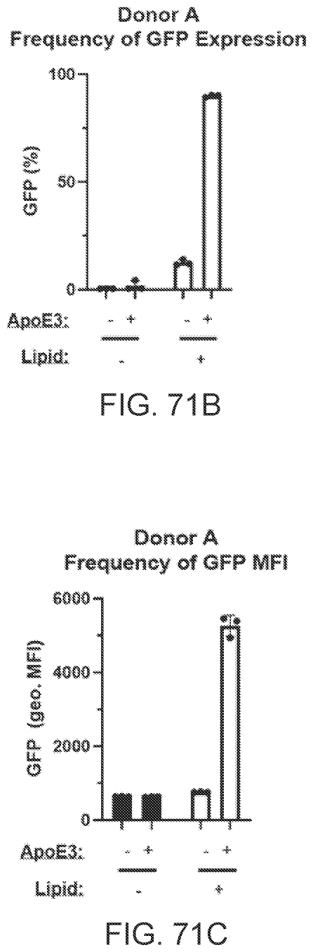

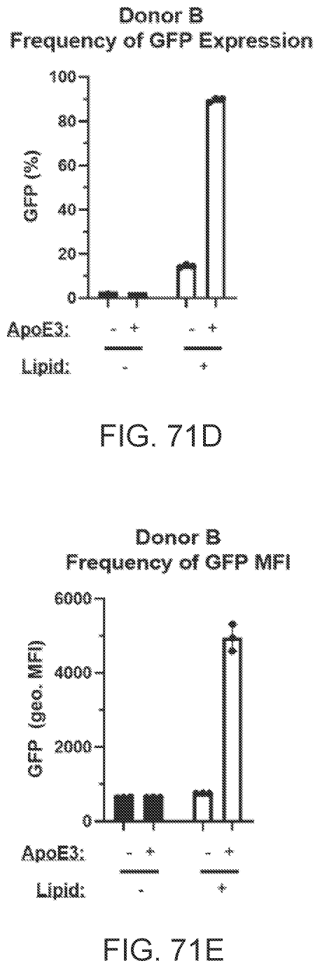

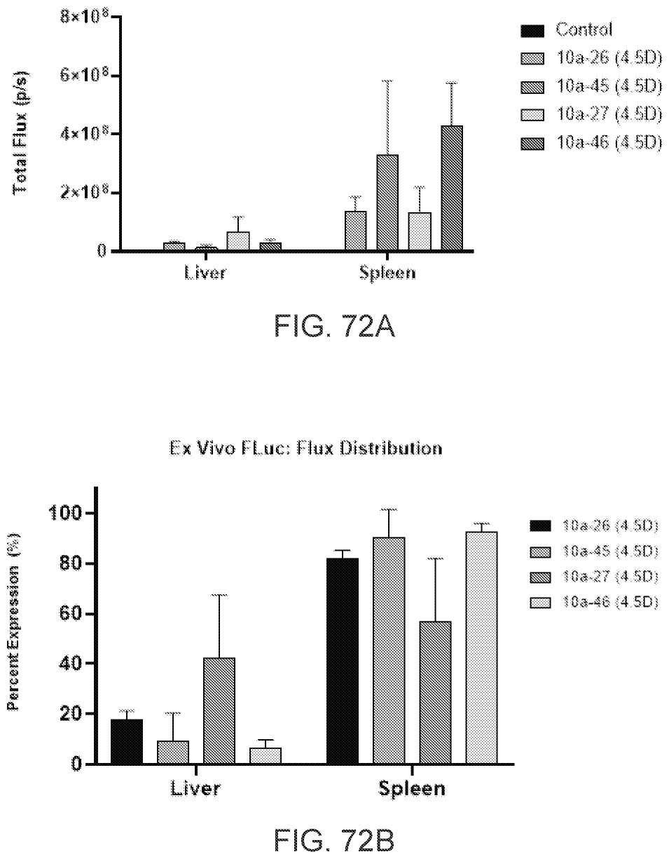

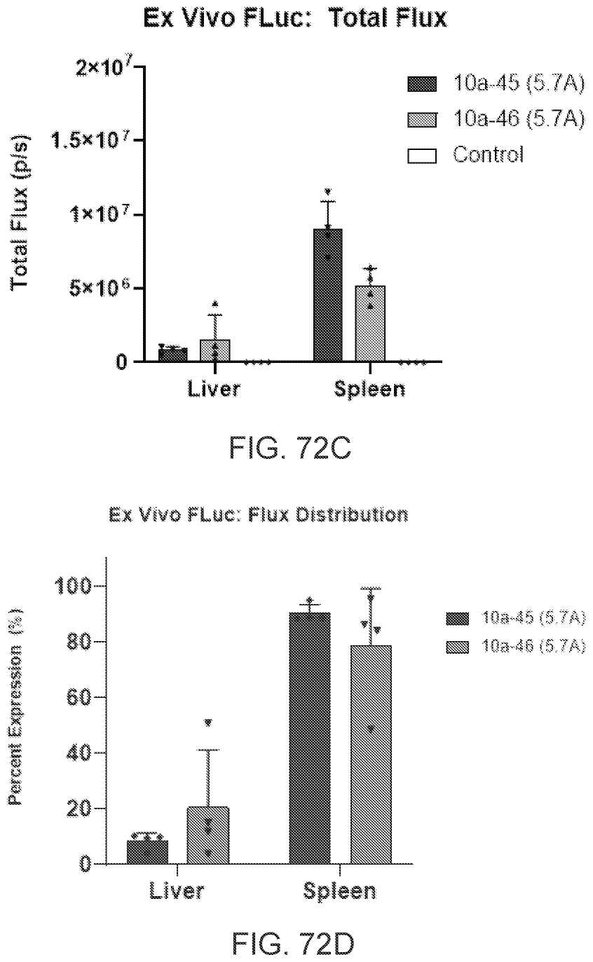

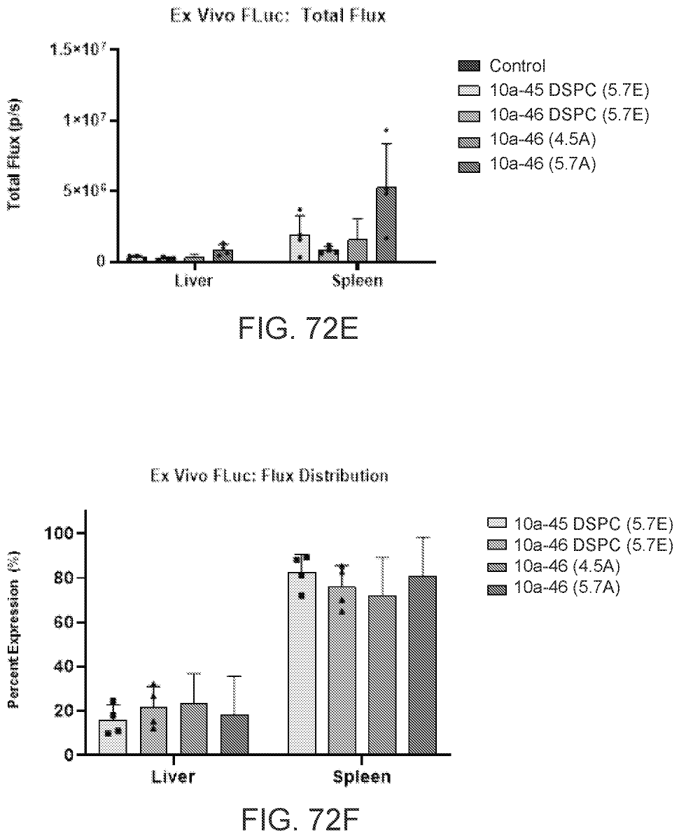

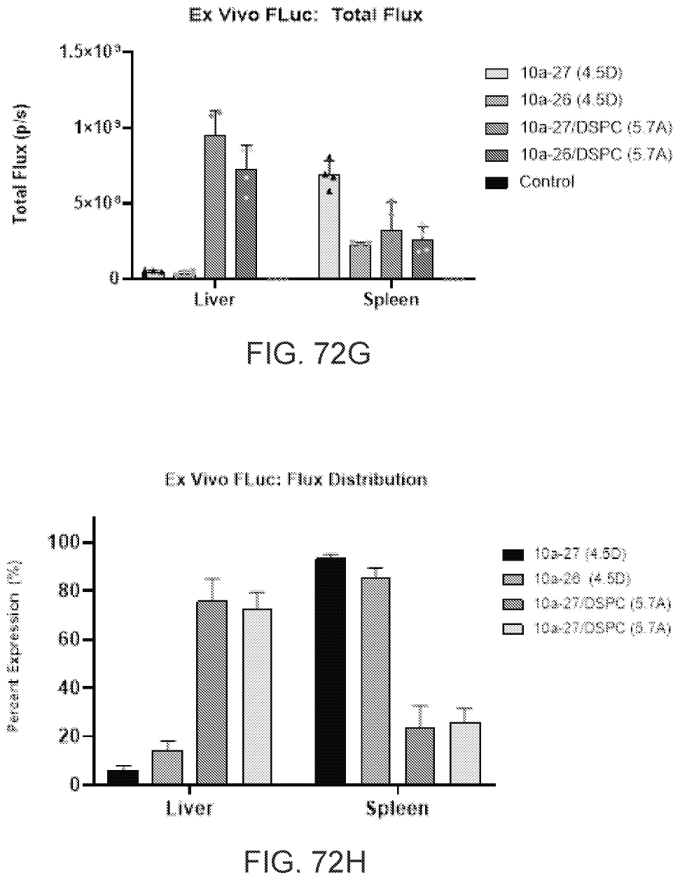

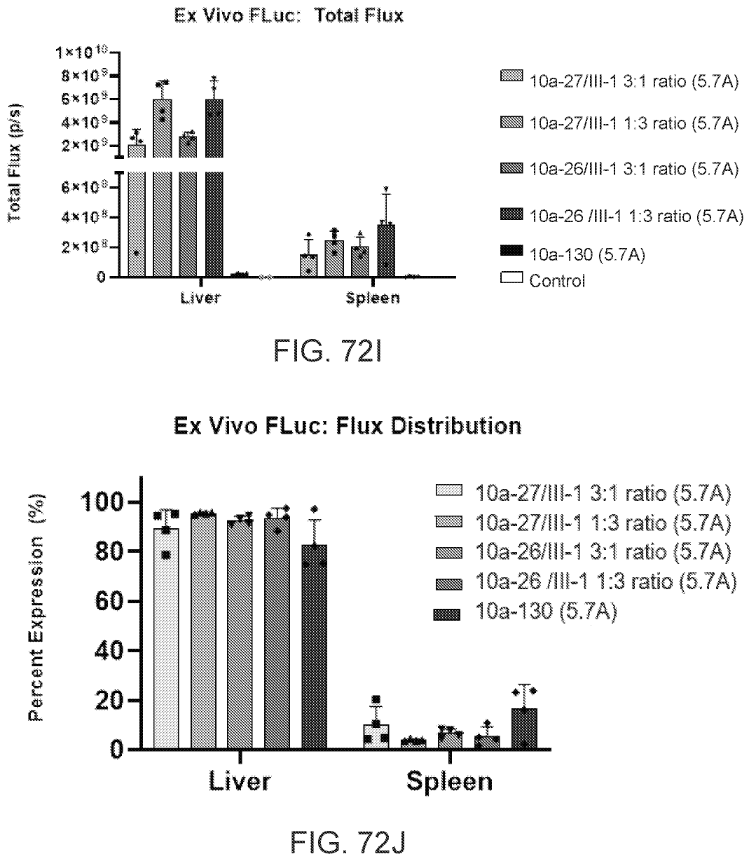

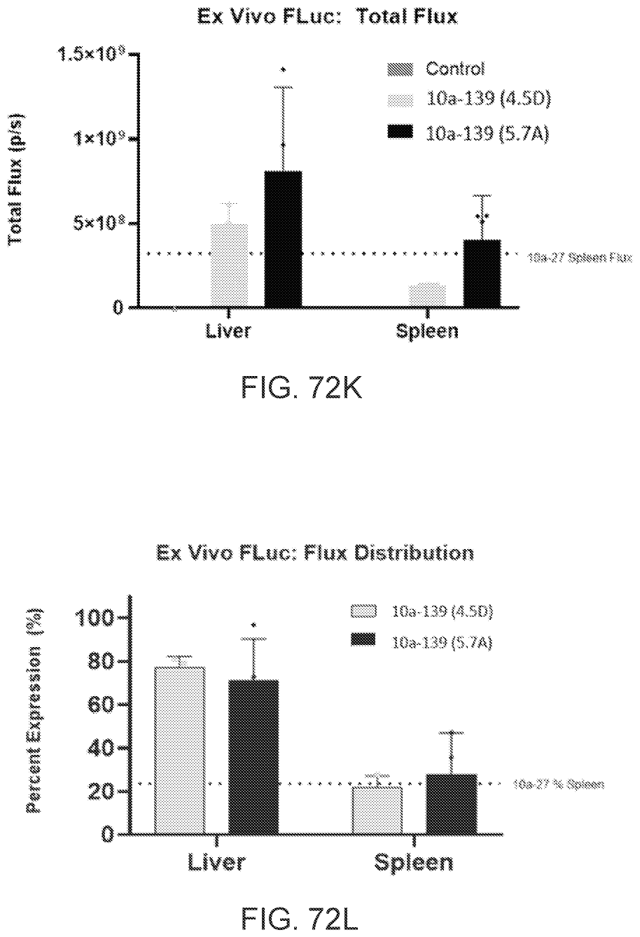

As shown by FIGS. 71A, 71B, 71C, 72D, and 71E, efficient LNP transfection can be mediated by ApoE3 on activated T-cells, followed by significant payload expression. These results were exhibited across multiple donors.

### Example 74

### Example 74A: Lipid Nanoparticle Formulation Procedure

A Zetasizer Nano ZS (Malvern Instruments Ltd, Malvern, Worcestershire, UK) can be used to determine the particle size, the polydispersity index (PDI) and the zeta potential of the transfer vehicle compositions in 1×PBS in determining particle size and 15 mM PBS in determining zeta potential.

Ultraviolet-visible spectroscopy can be used to determine the concentration of a therapeutic and/or prophylactic (e.g., RNA) in transfer vehicle compositions. 100 μL of the diluted formulation in 1×PBS is added to 900 μL of a 4:1 (v/v) mixture of methanol and chloroform. After mixing, the absorbance spectrum of the solution is recorded, for example, between 230 nm and 330 nm on a DU 800 spectrophotometer (Beckman Coulter, Beckman Coulter, Inc., Brea, CA). The concentration of therapeutic and/or prophylactic in the transfer vehicle composition can be calculated based on the extinction coefficient of the therapeutic and/or prophylactic used in the composition and on the difference between the absorbance at a wavelength of, for example, 260 nm and the baseline value at a wavelength of, for example, 330 nm.

For transfer vehicle compositions including RNA, a QUANT-IT™ RIBOGREEN® RNA assay (Invitrogen Corporation Carlsbad, CA) can be used to evaluate the encapsulation of RNA by the transfer vehicle composition. The samples are diluted to a concentration of approximately 5 μg/mL or 1 μg/mL in a TE buffer solution (10 mM Tris-HCl, 1 mM EDTA, pH 7.5). 50 μL of the diluted samples are transferred to a polystyrene 96 well plate and either 50 μL of TE buffer or 50 μL of a 2-4% Triton X-100 solution is added to the wells. The plate is incubated at a temperature of 37° C. for 15 minutes. The RIBOGREEN® reagent is diluted 1:100 or 1:200 in TE buffer, and 100 μL of this solution is added to each well. The fluorescence intensity can be measured using a fluorescence plate reader (Wallac Victor 1420 Multilablel Counter; Perkin Elmer, Waltham, MA) at an excitation wavelength of, for example, about 480 nm and an emission wavelength of, for example, about 520 nm. The fluorescence values of the reagent blank are subtracted from that of each of the samples and the percentage of free RNA is determined by dividing the fluorescence intensity of the intact sample (without addition of Triton X-100) by the fluorescence value of the disrupted sample (caused by the addition of Triton X-100).

### Example 74B: RNA Encapsulation, Total Flux, and Percent Expression In Vitro for Ionizable Lipid:DOPE:Cholesterol:DSPE-PEG(2000)Formulation Ratio of 62:4:33:1

Lipid nanoparticles were formulated using Lipid 10a-27, 10a-26, 10a-46, or 10a-45 in a ionizable lipid:DOPE:cholesterol:DSPE-PEG(2000) formulation ratio of 62:4:33:1 mol %, and encapsulate the RNA molecule at a lipid-nitrogen-to-phosphate ratio (N:P) of 4.5. Expression of the RNA was present in all formulations. There was a greater total flux and percent expression within the spleen as shown in FIGS. 72A and 72B respectively.

| | | | | Ionizable lipid: | | | |
|---|---|---|---|---|---|---|---|
| | | | | Helper lipid: | | | |
| | | | | Cholesterol: | Z- | | RNA |
| | Ionizable | Helper | | PEG-lipid | Average | | Encapsulation |
| Formulation | lipid | lipid | PEG-lipid | (mol %) | (nm) | PDI | Efficiency (%) |
| 10a-27 (4.5D) | Lipid 10a- | DOPE | DSPE- | 62:4:33:1 | 71 | 0.02 | 94 |
| | 27 | | PEG(2000) | | | | |
| 10a-26 (4.5D) | Lipid 10a- | DOPE | DSPE- | 62:4:33:1 | 71 | 0.04 | 92 |
| | 26 | | PEG(2000) | | | | |
| 10a-46 (4.5D) | Lipid 10a- | DOPE | DSPE- | 62:4:33:1 | 110 | 0.1 | 93 |
| | 26 | | PEG(2000) | | | | |
| 10a-45 (4.5D) | Lipid 10a- | DOPE | DSPE- | 62:4:33:1 | 157 | 0.13 | 83 |
| | 25 | | PEG(2000) | | | | |

### Example 74C: RNA Encapsulation, Total Flux, and Percent Expression In Vitro for Ionizable Lipid:DOPE:Cholesterol:DMG-PEG(2000)Formulation Ratio of 50:10:38.5:1.5

Lipid nanoparticles were formulated using Lipid 10a-46 or 10a-45 in a ionizable lipid:DOPE:cholesterol:DMG-PEG(2000) formulation ratio of 50:10:38.5:1.5 mol %, and encapsulate the RNA molecule at a lipid-nitrogen-to-phosphate ratio (N:P) of 5.7. Expression of the RNA was present in all formulations. There was a greater total flux and percent expression within the spleen as shown in FIGS. 72C and 72D respectively.

| | | | | Ionizable lipid: | | | |
|---|---|---|---|---|---|---|---|
| | | | | DOPE: | | | |
| | | | | Cholesterol: | Z- | | RNA |
| | Ionizable | Helper | | PEG-lipid | Average | | Encapsulation |
| Formulation | lipid | lipid | PEG-lipid | (mol %) | (nm) | PDI | Efficiency (%) |
| 10a-45 (5.7A) | Lipid | DOPE | DMG- | 50:10:38.5:1.5 | 74 | 0.04 | 95 |
| | 10a-45 | | PEG(2000) | | | | |
| 10a-46 (5.7A) | Lipid | DOPE | DMG- | 50:10:38.5:1.5 | 84 | 0.04 | 96 |
| | 10a-46 | | PEG(2000) | | | | |

### Example 74D: RNA Encapsulation, Total Flux, and Percent Expression In Vitro for Ionizable Lipid:DOPE:Cholesterol:DMG-PEG(2000) Formulation Ratio of 50:10:38.5:1.5 or for Ionizable Lipid DSPC:Cholesterol:C₁₄-PEG(2000) Formulation Ratio 35:16:46.2.5

Lipid nanoparticles were formulated using Lipid 10a-45 or 10a-46 in an ionizable lipid:DOPE:cholesterol:DMG-PEG(2000) formulation ratio of 50:10:38.5:1.5 mol % or in an ionizable lipid: DSPC:cholesterol:C₁₄-PEG(2000) formulation ratio of 35:16:46.2.5 mol %, and encapsulate the RNA molecule at a lipid-nitrogen-to-phosphate ratio (N:P) of 5.7 or 4.5. Expression of the RNA was present in all formulations. There was a greater total flux and percent expression within the spleen as shown in FIGS. 72E and 72F respectively.

| | | | | Ionizable lipid: | | | |
|---|---|---|---|---|---|---|---|
| | | | | Helper lipid: | | | |
| | | | | Cholesterol: | Z- | | RNA |
| | Ionizable | Helper | | PEG-lipid | Average | | Encapsulation |
| Formulation | lipid | lipid | PEG-lipid | (mol %) | (nm) | PDI | Efficiency (%) |
| 10a-45 DSPC | Lipid | DSPC | C₁₄- | 35:16:46.2.5 | 56 | 0.22 | 94 |
| (5.7E) | 10a-45 | | PEG(2000) | | | | |
| 10a-46 DSPC | Lipid | DSPC | C₁₄- | 35:16:46.2.5 | 68 | 0.02 | 95 |
| (5.7E) | 10a-46 | | PEG(2000) | | | | |
| 10a-46 (4.5A) | Lipid | DOPE | DMG- | 50:10:38.5:1.5 | 91 | 0.13 | 93 |
| | 10a-46 | | PEG(2000) | | | | |
| 10a-46 (5.7A) | Lipid | DOPE | DMG- | 50:10:38.5:1.5 | 76 | 0.06 | 93 |
| | 10a-46 | | PEG(2000) | | | | |

### Example 74E: RNA Encapsulation, Total Flux, and Percent Expression In Vitro for Ionizable Lipid:DOPE:Cholesterol:DSPE-PEG(2000) Formulation Ratio of 62:4:33:1 or for Ionizable Lipid:DSPC:Cholesterol:DMG-PEG(2000) Formulation Ratio of 50:10:38.5:1.5

Lipid nanoparticles were formulated using Lipid 10a-26 or 10a-27 in a ionizable lipid:DOPE:cholesterol:DSPE-PEG(2000) formulation ratio of 62:4:33:1 mol % (encapsulating the RNA molecule at a N:P ratio of 4.5) or ionizable lipid:DSPC:cholesterol:DMG-PEG(2000) formulation ratio of 50:10:38.5-1.5 mol % (encapsulating the RNA molecule at a N:P ratio of 5.7). Expression of the RNA was present in all formulations. There was a greater total flux and percent expression within the spleen as shown in FIGS. 72G and 72H respectively.

| | | | | Ionizable lipid: | | | |
|---|---|---|---|---|---|---|---|
| | | | | Helper lipid: | | | |
| | | | | Cholesterol: | Z- | | Encapsulation |
| | Ionizable | Helper | | PEG-lipid | Average | | Efficiency (EE) |
| Formulation | lipid | lipid | PEG-lipid | (mol %) | (nm) | PDI | (%) |
| 10a-27 (4.5D) | Lipid | DOPE | DSPE- | 62:4:33:1 | 82 | 0.06 | 94 |
| | 10a-27 | | PEG(2000) | | | | |
| 10a-26 (4.5D) | Lipid | DOPE | DSPE- | 62:4:33:1 | 68 | 0.08 | 91 |
| | 10a-26 | | PEG(2000) | | | | |
| 10a-27 DSPC | Lipid | DSPC | DMG- | 50:10:38.5:1.5 | 79 | 0.06 | 96 |
| (5.7A) | 10a-27 | | PEG(2000) | | | | |
| 10a-26 DSPC | Lipid | DSPC | DMG- | 50:10:38.5:1.5 | 79 | 0.05 | 93 |
| (5.7A) | 10a-26 | | PEG(2000) | | | | |

### Example 74F RNA Encapsulation, Total Flux, and Percent Expression In Vitro for Ionizable Lipid:DOPE:Cholesterol:DMG-PEG(2000) Formulation Ratio of 50:10:38.5:1.5

Lipid nanoparticles were formulated using Lipid 10a-26, 10a-27, or 10a-130 and/or Lipid 3-III-1 (represented by

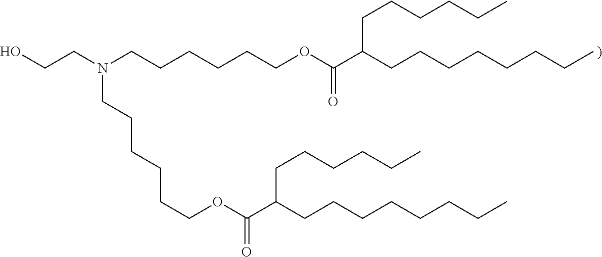

in a ionizable lipid: DSPC:cholesterol:DMG-PEG(2000) formulation ratio of 50:10:38.5:1.5 mol %) and encapsulate the RNA molecule at a N:P ratio of 5.7. Expression of the RNA was present in all formulations. There was a greater total flux and percent expression within the liver as shown in FIGS. 72I and 72J respectively.

TNS and the particle's pKa was also calculated. 5 μL of 60 μg/mL 2-(p-toluidino) naphthalene-6-sulfonic acid (TNS) and 5 μL of 30 μg of RNA/mL lipid nanoparticles were added in to wells with HEPES buffer ranging from pH 2-12. The mixture was then shaken at room temperature for 5 minutes, and read for fluorescence (excitation 322 nm, emission 431 nm) using a plate reader. The inflection point of the fluorescence signal was calculated to determine the particle's pKₐ.

| | | | | Ionizable | | | | | |
|---|---|---|---|---|---|---|---|---|---|
| | | | | lipid:Helper | | | | | |
| | | | | lipid: | | | | | |
| | | | | Cholesterol: | | | | | |
| | Ionizable | Helper | | PEG-lipid | Z- | | EE | Conc. | |
| Formulation | lipid | lipid | PEG-lipid | (mol %) | Average | PDI | (%) | (ug/mL) | pKa |
| 10a-27/III-1 | Lipid 10a-27/ | DOPE | DMG- | 50:10:38.5:1.5 | 74 | 0 | 96 | 55.8 | 7.4 |
| (5.7A) | Lipid 3-III- | | PEG(2000) | | | | | | |
| | 13:1 ratio | | | | | | | | |
| 10a-27III-1 | Lipid 10a-27/ | DOPE | DMG- | 50:10:38.5:1.5 | 85 | 0.1 | 93 | 51.9 | 6.3 |
| (5.7A) | Lipid 3-III- | | PEG(2000) | | | | | | |
| | 11:3 ratio | | | | | | | | |
| 10a-26/III-1 | Lipid 10a-26/ | DOPE | DMG- | 50:10:38.5:1.5 | 87 | 0.1 | 94 | 51.7 | 6.8 |
| (5.7A) | Lipid 3-III- | | PEG(2000) | | | | | | |
| | 13:1 ratio | | | | | | | | |
| 10a-26/III-1 | Lipid 10a-26/ | DOPE | DMG- | 50:10:38.5:1.5 | 97 | 0.1 | 90 | 53.1 | 6.2 |
| (5.7A) | Lipid 3-III- | | PEG(2000) | | | | | | |
| | 11:3 ratio | | | | | | | | |
| 10a-130 | Lipid 10a- | DOPE | DMG- | 50:10:38.5:1.5 | 60 | 0.1 | 89 | 53.8 | 6.7 |
| (5.7A) | 130 | | PEG(2000) | | | | | | |

### Example 74G: RNA Encapsulation, Total Flux, and Percent Expression In Vitro for Ionizable Lipid:DOPE:Cholesterol:DSPE-PEG(2000) Formulation Ratio of 62:4:33:1 or for Ionizable Lipid:DSPC:Cholesterol:DMG-PEG(2000) Formulation Ratio of 50:10:38.5:1.5

Lipids nanoparticles were formulated using Lipid 10a-139 in a ionizable lipid:DOPE-cholesterol: DSPE-PEG(2000) formulation ratio of 62-4-33-1 mol % (encapsulating the RNA molecule at a N:P ratio of 4.5) or ionizable lipid:DOPE:cholesterol:DMG-PEG(2000) formulation ratio of 50:10:38.5:1.5 mol % (encapsulating the RNA molecule at a N:P ratio of 5.7). Expression of the RNA was present in all formulations. There was a greater total flux and percent expression within the liver as shown in FIGS. 72K and 72L respectively.

| | | | | Ionizable lipid:Helper | Z- | | |
|---|---|---|---|---|---|---|---|
| | Ionizable | Helper | | lipid:Cholesterol:PEG- | Average | | EE |
| Formulation | lipid | lipid | PEG-lipid | lipid (mol %) | (nm) | PDI | (%) |
| 10a-139 | Lipid 10a- | DOPE | DSPE- | 62:4:33:1 | 93 | 0.01 | 79 |
| (4.5D) | 139 | | PEG(2000) | | | | |
| 10a-139 | Lipid 10a- | DSPC | DMG- | 50:10:38.5:1.5 | 122 | 0.02 | 95 |
| (5.7A) | 139 | | PEG(2000) | | | | |

### Example 75

This example illustrates expression of SARS-CoV2 spike protein expression in vitro. Circular RNA encoding SARS-CoV2 stabilized spike protein was transfected into 293 cells using MessengerMax Transfection Reagent. 24 hours after transfection, the 293 cells were stained with a CR3022 anti-spike primary antibody and APC-labeled secondary antibody.

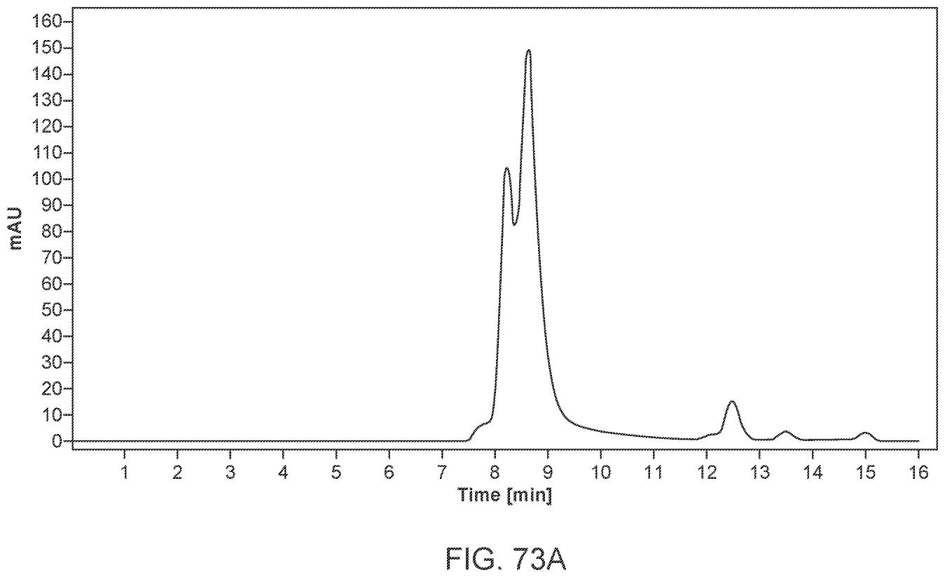

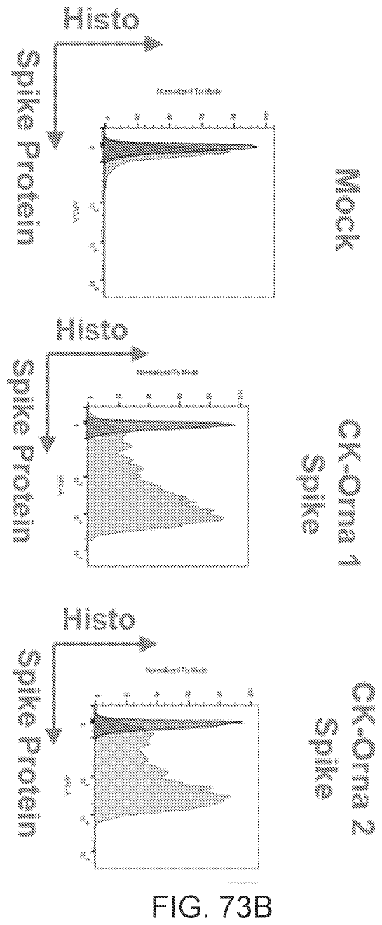

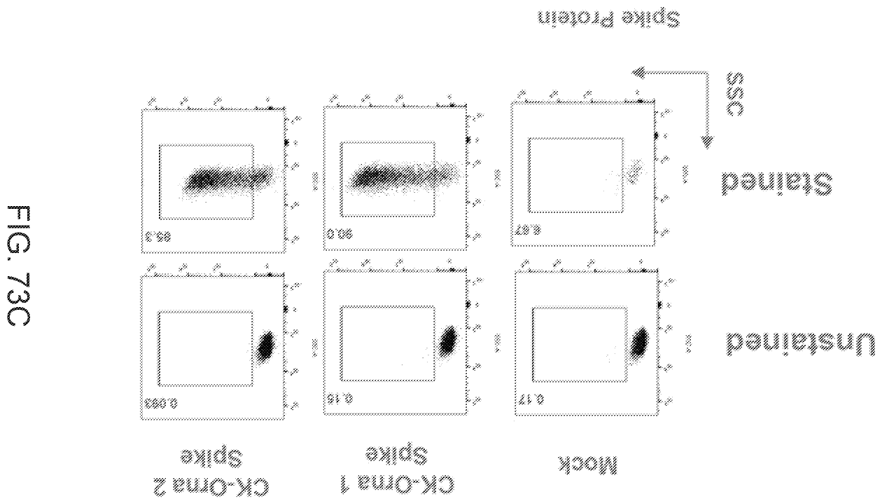

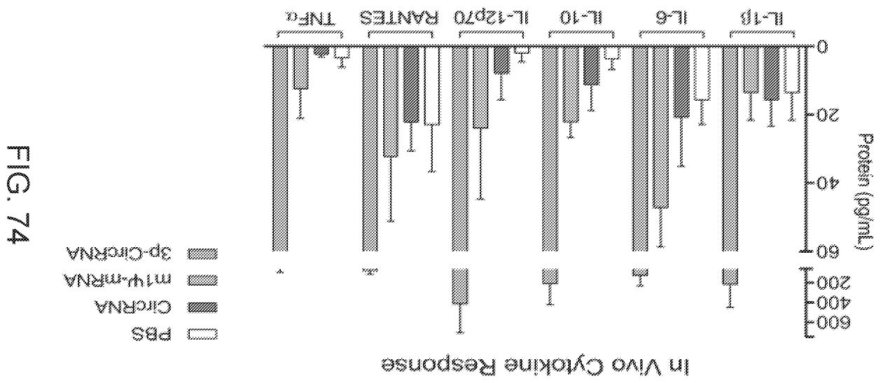

FIG. 73A shows circularization efficiency of roughly 4.5 kb SARS-Cov2 stabilized spike protein-encoding RNA resulting from an in vitro transcription reaction. FIG. 73B and FIG. 73C show SARS-CoV2 stabilized spike protein expression on 293 cells after the circular RNA transfection with MessengerMax Transfection Reagent relative to mock transfected cells.

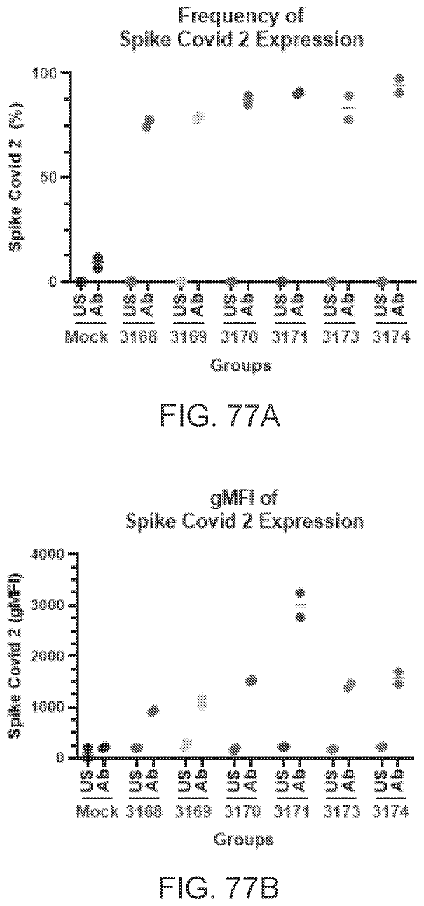

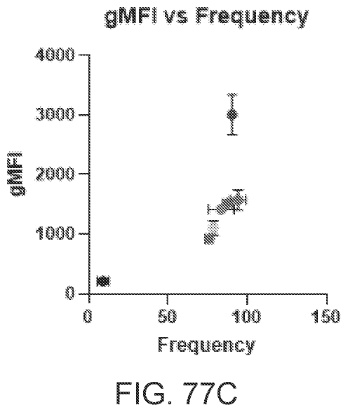

FIG. 77A and FIG. 77B show SARS-CoV2 stabilized spike protein expression by percentage of cells and gMFI on 293 cells after transfection of a panel of circular RNAs, containing variable IRES sequences, codon optimized coding regions, and stabilized SARS-CoV2 spike proteins, using MessengerMax Transfection Reagent. FIG. 77C shows the relationship between MFI and percentage.

### Example 76

This example shows in vivo cytokine response after IV injection of 0.2 mg/kg circRNA preparations encapsulated in a lipid nanoparticle formulation. circRNA splicing reactions synthesized with GTP as a precursor RNA initiator and splicing nucleotide incited greater cytokine responses than purified circRNA and linear m1ψ-mRNA due to the presence of triphosphorylated 5′ termini in the splicing reaction. Levels of IL-1β, IL-6, IL-10, IL-12p70, RANTES, TNFα were measured from blood drawn 6 hours following intravenous injection of the LNP-formulated circRNA preparation. Mice injected with PBS were used as a control.

As seen in FIG. 74, circRNA splicing reactions synthesized with GTP as a precursor RNA initiator and splicing nucleotide incite greater cytokine responses than purified circRNA and linear m1ψ-mRNA due to the presence of triphosphorylated 5′ termini in the splicing reaction.

### Example 77

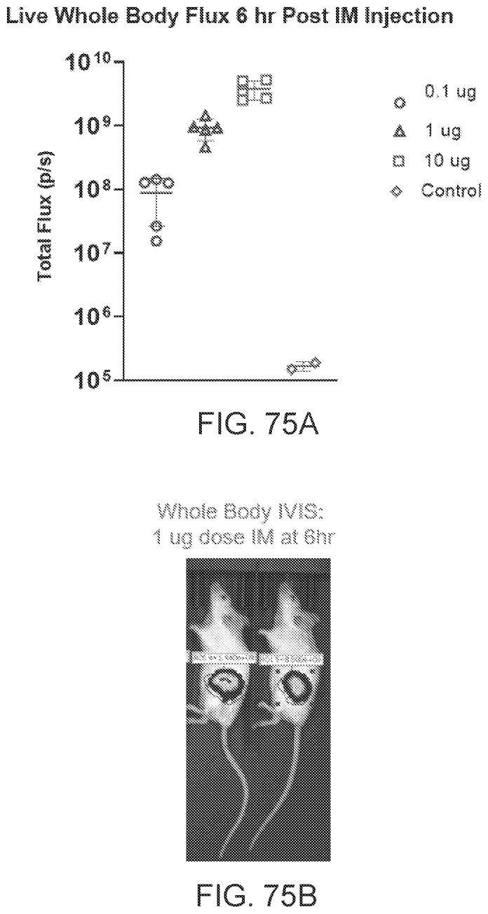

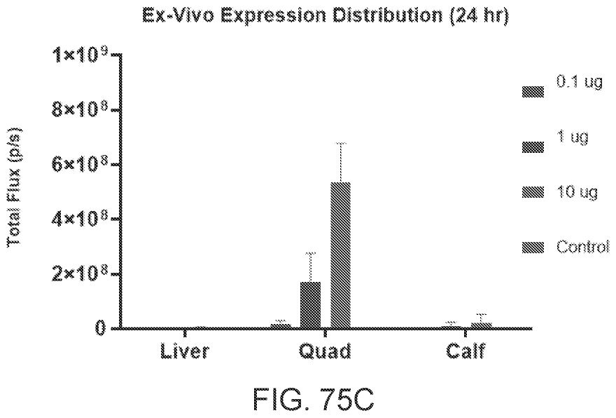

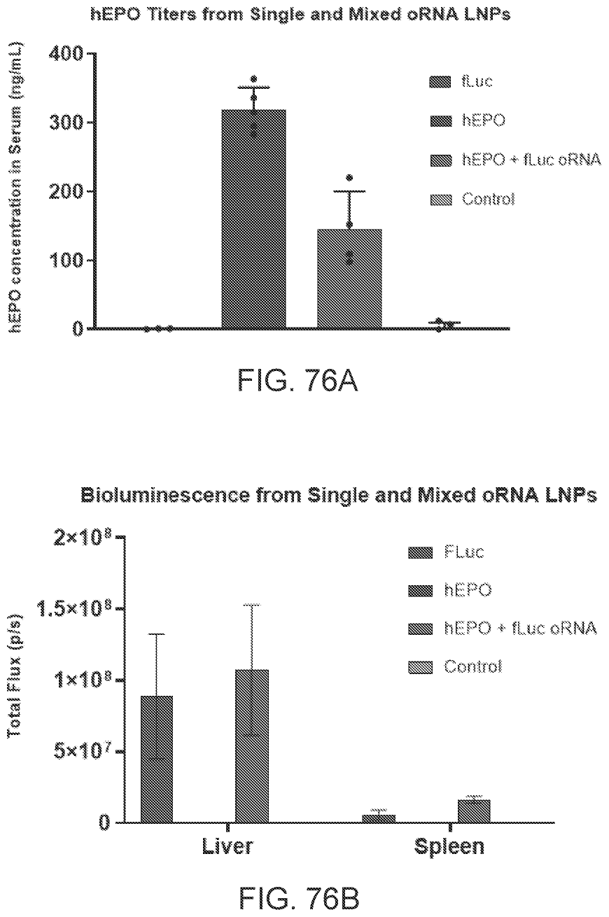

This example illustrates intramuscular delivery of varying doses of lipid nanoparticle containing circular RNAs. Mice were dosed with either 0.1 μg, 1 μg, or 10 μg of circRNA formulated in lipid nanoparticles. Whole body IVIS imagine was conducted at 6 hours following an injection of luciferin (FIG. 75A and FIG. 75B). F& vivo IVIS imaging was conducted at 24-hour. Flux values for liver, quad, and calf are shown in FIG. 75C. FIG. 76B and FIG. 76C show that the expression of the circular RNA is present in the muscle tissue of the mice.

### Example 78

This example illustrates expression of multiple circular RNAs in LNP formulations. Circular RNA constructs encoding either hEPO or fLuc were dosed in a single and mixed set of LNPs. hEPO concentration in the serum (FIG. 76A) and total flux by IVIS imaging (FIG. 76B) was determined. The results show that the circular RNA hEPO or fLuc constructs individually formulated or co-formulated expressed protein efficiently.

### INCORPORATION BY REFERENCE

All publications, patents, and patent applications mentioned in this specification are herein incorporated by reference to the same extent as if each individual publication, patent, or patent application was specifically and individually indicated as being incorporated by reference herein.

What is claimed is:

1. A circular RNA polynucleotide comprising, in the following order,
a. a 3′ group I intron fragment,
b. an Internal Ribosome Entry Site (IRES),
c. an expression sequence encoding one or more antigens, adjuvants, antigen-like or adjuvant-like polypeptides, or fragments thereof, and
d. a 5′ group I intron fragment.

2. A circular RNA polynucleotide comprising, in the following order,
a. a 3′ group I intron fragment,
b. an Internal Ribosome Entry Site (IRES),
c. a non-coding expression sequence, and
d. a 5′ group I intron fragment.

3. A circular RNA polynucleotide produced from transcription of a vector comprising, in the following order,
a. a 5′ duplex forming region,
b. a 3′ group I intron fragment,
c. an Internal Ribosome Entry Site (IRES),
d. an expression sequence encoding one or more antigens, adjuvants, antigen-like or adjuvant-like polypeptides, or fragments thereof,
e. a 5′ group I intron fragment, and
f. a 3′ duplex forming region.

4. A circular RNA polynucleotide produced from the transcription of a vector comprising, in the following order,
a. a 5′ duplex forming region,
b. a 3′ group I intron fragment,
c. an Internal Ribosome Entry Site (IRES),
d. a non-coding expression sequence,
e. a 5′ group I intron fragment, and
f. a 3′ duplex forming region.

5. The circular RNA polynucleotide of claim 3 or 4, comprising a first spacer between the 5′ duplex forming region and the 3′ group I intron fragment, and a second spacer between the 5′ group 1 intron fragment and the 3′ duplex forming region.

6. The circular RNA polynucleotide of claim 5, wherein the first and second spacers each have a length of about 10 to about 60 nucleotides.

7. The circular RNA polynucleotide of any one of claims 3-6, wherein the first and second duplex forming regions each have a length of about 9 to about 19 nucleotides.

8. The circular RNA polynucleotide of any one of claims 3-6, wherein the first and second duplex forming regions each have a length of about 30 nucleotides.

9. The circular RNA polynucleotide of any one of claims 1-8, wherein the IRES has a sequence of an IRES from Taura syndrome virus, Triatoma virus, Theiler's encephalomyelitis virus, Simian Virus 40, Solenopsis invicta virus 1, Rhopalosiphum padi virus, Reticuloendotheliosis virus, Human poliovirus 1, Plautia stali intestine virus, Kashmir bee virus, Human rhinovirus 2, Homalodisca coagulata virus-1, Human Immunodeficiency Virus type 1, Homalodisca coagulata virus-1, Himetobi P virus, Hepatitis C virus, Hepatitis A virus, Hepatitis GB virus, Foot and mouth disease virus, Human enterovirus 71, Equine rhinitis virus, Ectropis obliqua picorna-like virus, Encephalomyocarditis virus, Drosophila C Virus, Human coxsackievirus B3, Crucifer tobamovirus, Cricket paralysis virus, Bovine viral diarrhea virus 1, Black Queen Cell Virus, Aphid lethal paralysis virus, Avian encephalomyelitis virus, Acute bee paralysis virus, Hibiscus chlorotic ringspot virus, Classical swine fever virus, Human FGF2, Human SFTPA1, Human AML1/RUNX1, Drosophila antennapedia, Human AQP4, Human AT1R, Human BAG-1, Human BCL2, Human BiP, Human c-IAP1, Human c-myc, Human eIF4G, Mouse NDST4L, Human LEF1, Mouse HIF1 alpha, Human n.myc, Mouse Gtx, Human p27kipl, Human PDGF2/c-sis, Human p53, Human Pim-1, Mouse Rbm3, Drosophila reaper, Canine Scamper, Drosophila Ubx, Human UNR, Mouse UtrA, Human VEGF-A, Human XIAP, Drosophila hairless, S. cerevisiae TFIID, S. cerevisiae YAP1, tobacco etch virus, turnip crinkle virus, EMCV-A, EMCV-B, EMCV-Bf, EMCV-Cf, EMCV pEC9, Picobirnavirus, HCV QC64, Human Cosavirus E/D, Human Cosavirus F, Human Cosavirus JMY, Rhinovirus NAT001, HRV14, HRV89, HRVC-02, HRV-A21, Salivirus A SH1, Salivirus FHB, Salivirus NG-J1, Human Parechovirus 1, Crohivirus B, Yc-3, Rosavirus M-7, Shanbavirus A, Pasivirus A, Pasivirus A 2, Echovirus E14, Human Parechovirus 5, Aichi Virus, Hepatitis A Virus HA16, Phopivirus, CVA10, Enterovirus C, Enterovirus D, Enterovirus J, Human Pegivirus 2, GBV-C GT 110, GBV-C K1737, GBV-C Iowa, Pegivirus A 1220, Pasivirus A 3, Sapelovirus, Rosavirus B, Bakunsa Virus, Tremovirus A, Swine Pasivirus 1, PLV-CHN, Pasivirus A, Sicinivirus, Hepacivirus K, Hepacivirus A, BVDV1, Border Disease Virus, BVDV2, CSFV-PK15C, SF573 Dicistrovirus, Hubei Picorna-like Virus, CRPV, Salivirus A BN5, Salivirus A BN2, Salivirus A 02394, Salivirus A GUT, Salivirus A CH, Salivirus A SZ1, Salivirus FHB, CVB3, CVB1, Echovirus 7, CVB5, EVA71, CVA3, CVA12, EV24 or an aptamer to eIF4G.

10. The circular RNA polynucleotide of any one of claims 1-9, consisting of natural nucleotides.

11. The circular RNA polynucleotide of any one of claims 1-9, wherein the expression sequence is codon optimized.

12. The circular RNA polynucleotide of any one of claims 1-11, wherein the circular RNA polynucleotide is from about 100 nucleotides to about 10 kilobases in length.

13. The circular RNA polynucleotide of any one of the claims 1-12, having an in vivo duration of therapeutic effect in humans of at least about 20 hours.

14. The circular RNA polynucleotide of any one of claims 1-13, having a functional half-life of at least about 20 hours.

15. The circular RNA polynucleotide of any one of claims 1-14, having a duration of therapeutic effect in a human cell greater than or equal to that of an equivalent linear RNA polynucleotide comprising the same expression sequence.

16. The circular RNA polynucleotide of any one of claims 1-15, having a functional half-life in a human cell greater than or equal to that of an equivalent linear RNA polynucleotide comprising the same expression sequence.

17. The circular RNA polynucleotide of any one of claims 1-16, having an i vivo duration of therapeutic effect in humans greater than that of an equivalent linear RNA polynucleotide having the same expression sequence.

18. The circular RNA polynucleotide of any one of claims 1-17, having an in vivo functional half-life in humans greater than that of an equivalent linear RNA polynucleotide having the same expression sequence.

19. The circular RNA polynucleotide of any one of claims 1, 3, and 5-18, wherein the adjuvant or adjuvant-like polypeptide is selected from the group comprising toll-like receptor ligand, cytokine, FLt3-ligand, antibody, chemokines, chimeric protein, endogenous adjuvant released from a dying tumor, and checkpoint inhibition proteins.

20. The circular RNA polynucleotide of any one of claims 1, 3, and 5-19, wherein the adjuvant or adjuvant-like polypeptide is selected from the group comprising BCSP31, MOMP, FomA, MymA, ESAT6, PorB, PVL, Porin, OmpA, PepO, OmpU, Lumazine synthase, Omp16, Omp19, CobT, RpfE, Rv0652, HBHA, NhhA, DnaJ, Pneumolysin, Falgellin, IFN-alpha, IFN-gamma, IL-2, IL-12, IL-15, IL-18, IL-21, GM-CSF, IL-1b, IL-6, TNF-a, IL-7, IL-17, IL-1Beta, anti-CTLA4, anti-PD1, anti-41BB, PD-L1, Tim-3, Lag-3, TIGIT, GITR, and andti-CD3.

21. The circular RNA polynucleotide of any one of claims 1, 3, and 5-20, wherein the adjuvant or adjuvant-like polypeptide is selected from Table 10.

22. An RNA polynucleotide comprising, in the following order, a 3′ intron fragment and a triphosphorylated 5′ terminus.

23. The RNA polynucleotide of claim 22, comprising a 5′ spacer located upstream to the 3′ intron fragment and downstream from the triphosphorylated 5′ terminus.

24. An RNA polynucleotide comprising, in the following order, a 3′ intron fragment and a monophosphorylated 5′ terminus.

25. The RNA polynucleotide of claim 24, comprising a 5′ spacer located upstream to the 3′ intron fragment and downstream from the monophosphorylated 5′ terminus.

26. An RNA polynucleotide, comprising a 5′ intron fragment and a triphosphorylated 5′ terminus.

27. The RNA polynucleotide of claim 26, comprising a 5′ spacer located downstream to the 5′ intron fragment.

28. An RNA polynucleotide, comprising a 5′ intron fragment and a monophosphorylated 5′ terminus.

29. The RNA polynucleotide of claim 28, comprising a 5′ spacer located downstream to the 5′ intron fragment.

30. The RNA polynucleotide of any one of claims 22-29, further comprising a polyA purification tag.

31. The RNA polynucleotide of any one of claims 22-30, further comprising an initiation sequence.

32. The circular RNA polynucleotide of claim 3 or 4, wherein the vector further comprises a triphosphorylated 5′ terminus.

33. The circular RNA polynucleotide of claim 3 or 4, wherein the vector further comprises a monophosorylated 5′ terminus.

34. The RNA polynucleotide of any one of claims 24-25 and 28-29, further comprising a triphosphorylated 5′ terminus.

35. The RNA polynucleotide of any one of claims 22-23 and 26-27, further comprising a monophosporylated 5′ terminus.

36. An RNA preparation comprising:
a. the circular RNA polynucleotide of claim 1, claim 2, or both; and
b. a linear RNA polynucleotide comprising, at least one of the following:
i. a 3′ intron polynucleotide comprising a monophosphorylated 5′ terminus and a 3′ intron fragment;
ii. a 5′ intron polynucleotide comprising a monophosphorylated 5′ terminus and a 5′ intron fragment;
iii. a 3′ intron polynucleotide comprising a triphosphorylated 5′ terminus and a 3′ intron fragment; and
iv. a 5′ intron polynucleotide comprising a triphosphorylated 5′ terminus and a 3′ intron fragment,
wherein the circular RNA polynucleotide comprises at least 90% of the RNA preparation.

37. The RNA preparation of claim 36, wherein the 3′ intron polynucleotide or 5′ intron polynucleotide comprises a spacer.

38. The RNA preparation of claim 36, wherein the 3′ intron polynucleotide or 5′ intron polynucleotide comprises a polyA sequence.

39. The RNA preparation of any one of claims 36-38, wherein the 3′ intron polynucleotide or 5′ intron polynucleotide comprises a UTR.

40. The RNA preparation of any one of claim 39, wherein the 3′ intron polynucleotide or 5′ intron polynucleotide comprises an IRES.

41. A pharmaceutical composition comprising a circular RNA polynucleotide of any one of claims 1-21, a diluent, and optionally a salt buffer.

42. A pharmaceutical composition comprising an RNA preparation of any one of claims 36-40, a diluent, and optionally a salt buffer.

43. A pharmaceutical composition comprising a circular RNA polynucleotide of any one of claims 1-21, and a polycationic, cationic, or polymeric compound.

44. A pharmaceutical composition comprising an RNA preparation of any one of claims 36-40, and a polycationic, cationic, or polymeric compound.

45. The pharmaceutical composition of claim 43 or 44, wherein the polycationic or cationic compound is selected from the group consisting of: cationic peptides or proteins, basic polypeptides, cell penetrating peptides (CPPs), Tat-derived peptides, Penetratin, VP22 derived or analog peptides, Pestivirus Erns, HSV, VP22 (Herpes simplex), MAP, KALA or protein transduction domains (PTDs), PpT620, proline-rich peptides, arginine-rich peptides, lysine-rich peptides, MPG-peptide(s), Pep-1, L-oligomers, Calcitonin peptide(s), Antennapedia-derived peptides, pAntp, pisl, FGF, Lactoferrin, Transportan, Buforin-2, Bac715-24, SynB, SynB(1), pVEC, hCT-derived peptides, SAP, histones, cationic polysaccharides, cationic polymers, cationic lipids, dendrimers, polyimine, polyallylamine, oligofectamine, or cationic or polycationic polymers, sugar backbone based polymers, silan backbone based polymers, modified polyaminoacids, modified acrylates, modified polybetaminoester (PBAE), modified amidoamines, dendrimers, blockpolymers consisting of a combination of one or more cationic blocks and of one or more hydrophilic or hydrophobic blocks.

46. The pharmaceutical composition of claim 43 or 44, wherein the polymeric compound is selected from the group consisting of: polyamines, polyethers, polyamides, polyesters, polycarbamates, polyureas, polycarbonates, polystyrenes, polyimides, polysulfones, polyurethanes, polyacetylenes, polyethylenes, polyethyleneimines, polyisocyanates, polyacrylates, polymethacrylates, polyacrylonitriles, and polyarylates. For example, a polymer may include poly(caprolactone) (PCL), ethylene vinyl acetate polymer (EVA), poly(lactic acid) (PLA), poly(L-lactic acid) (PLLA), poly(glycolic acid) (PGA), poly(lactic acid-co-glycolic acid) (PLGA), poly(L-lactic acid-co-glycolic acid) (PLLGA), poly(D,L-lactide) (PDLA), poly(Llactide) (PLLA), poly(D,L-lactide-co-caprolactone), poly(D,L-lactide-co-caprolactone-coglycolide), poly(D,L-lactide-co-PEO-co-D,L-lactide), poly(D,L-lactide-co-PPO-co-D,L-lactide), polyalkyl cyanoacrylate, polyurethane, poly-L-lysine (PLL), hydroxypropyl methacrylate (HPMA), polyethyleneglycol, poly-L-glutamic acid, poly(hydroxy acids), polyanhydrides, polyorthoesters, poly(ester amides), polyamides, poly(ester ethers), polycarbonates, polyalkylenes such as polyethylene and polypropylene, polyalkylene glycols such as poly(ethylene glycol) (PEG), polyalkylene oxides (PEO), polyalkylene terephthalates such as poly(ethylene terephthalate), polyvinyl alcohols (PVA), polyvinyl ethers, polyvinyl esters such as poly(vinyl acetate), polyvinyl halides such as poly(vinyl chloride) (PVC), polyvinylpyrrolidone (PVP), polysiloxanes, polystyrene, polyurethanes, derivatized celluloses such as alkyl celluloses, hydroxyalkyl celluloses, cellulose ethers, cellulose esters, nitro celluloses, hydroxypropylcellulose, carboxymethylcellulose, polymers of acrylic acids, such as poly(methyl(meth)acrylate) (PMMA), poly(ethyl(meth)acrylate), poly(butyl(meth)acrylate), 363 5 10 15 20 25 30 35 WO 2021/076805 PCT/US2020/055844 poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), poly(octadecyl acrylate) and copolymers and mixtures thereof, polydioxanone and its copolymers, polyhydroxyalkanoates, polypropylene fiimarate, polyoxymethylene, poloxamers, poloxamines, poly(ortho)esters, poly(butyric acid), poly(valeric acid), poly(lactide-co-caprolactone), trimethylene carbonate, poly(N-acryloylmorpholine) (PAcM), poly(2-methyl-2-oxazoline) (PMOX), poly(2-ethyl-2-oxazoline) (PEOZ), and polyglycerol.

47. The pharmaceutical composition of claim 43 or 44, wherein the polycationic or cationic compound is selected from the group comprising: protamine, nucleoline, spermine or spermidine, poly-L-lysine (PLL), polyarginine, HIV-binding peptides, HIV-1 Tat (HIV), polyethyleneimine (PEI), DOTMA: [1-(2,3-sioleyloxy)propyl)]-N,N,N-trimethylammonium chloride, DMRIE, di-C14-amidine, DOTIM, SAINT, DC-Chol, BGTC, CTAP, DOPC, DODAP, DOPE: Dioleyl phosphatidylethanol-amine, DOSPA, DODAB, DOIC, DMEPC, DOGS: Dioctadecylamidoglicylspermin, DIMRI: Dimyristooxypropyl dimethyl hydroxyethyl ammonium bromide, DOTAP: dioleoyloxy-3-(trimethylammonio)propane, DC-6-14: O,O-ditetradecanoyl-N-.alpha.-trimethylammonioacetyl)diethanolamine chloride, CLIP 1: rac-[(2,3-dioctadecyloxypropyl)(2-hydroxyethyl)]-dimethylammonium chloride, CLIP6: rac-[2(2,3-dihexadecyloxypropyloxymethyloxy)ethyl]-trimethylammonium, CLIP9: rac-[2(2,3-dihexadecyloxypropyloxysuccinyloxy)ethyl]-trimethylammonium, beta-aminoacid-polymers or reversed polyamides, PVP (poly(N-ethyl-4-vinylpyridinium bromide)), pDMAEMA (poly(dimethylaminoethyl methylacrylate)), pAMAM (poly(amidoamine)), diamine end modified 1,4 butanediol diacrylate-co-5-amino-1-pentanol polymers, polypropylamine dendrimers or pAMAM based dendrimers, polyimine(s), PEI: poly(ethyleneimine), poly(propyleneimine), polyallylamine, cyclodextrin based polymers, dextran based polymers, chitosan, and PMOXA-PDMS copolymers.

48. A pharmaceutical composition comprising a circular RNA polynucleotide of any one of the claims 1-21, a nanoparticle, and optionally, a targeting moiety operably connected to the nanoparticle.

49. A pharmaceutical composition comprising an RNA preparation of any one of claims 36-40, a nanoparticle, and optionally, a target moiety operably connected to the nanoparticle.

50. The pharmaceutical composition of claim 48 or 49, wherein the nanoparticle is a lipid nanoparticle, a core-shell nanoparticle, a biodegradable nanoparticle, a biodegradable lipid nanoparticle, a polymer nanoparticle, or a biodegradable polymer nanoparticle.

51. The pharmaceutical composition of any one of claims 41-50, comprising a targeting moiety, wherein the targeting moiety mediates receptor-mediated endocytosis or direct fusion into selected cells of a selected cell population or tissue in the absence of cell isolation or purification.

52. The pharmaceutical composition of any one of claims 48-51, wherein the targeting moiety is a scFv, nanobody, peptide, minibody, polynucleotide aptamer, heavy chain variable region, light chain variable region or fragment thereof.

53. The pharmaceutical composition of any one of claims 41-5257-57, wherein the circular RNA polynucleotide or RNA preparation is in an amount effective to treat or prevent an infection in a human subject in need thereof.

54. The pharmaceutical composition of any one of claims 41-53, wherein the pharmaceutical composition has an enhanced safety profile when compared to a pharmaceutical composition comprising vectors comprising exogenous DNA encoding the same antigen.

55. The pharmaceutical composition of any one of claims 41-54, wherein less than 1%, by weight, of the polynucleotides in the composition are double stranded RNA, DNA splints, or triphosphorylated RNA.

56. The pharmaceutical composition of any one of claims 41-55, wherein less than 1%, by weight, of the polynucleotides and proteins in the pharmaceutical composition are double stranded RNA, DNA splints, triphosphorylated RNA, phosphatase proteins, protein ligases, and capping enzymes.

57. The pharmaceutical composition of any one of claims 48-56, wherein the nanoparticle comprises one or more cationic lipids selected from the group C12-200, MC3, DLinDMA, DLinkC2DMA, cKK-E12, ICE (Imidazol-based), HGT5000, HGT5001, DODAC, DDAB, DMRIE, DOSPA, DOGS, DODAP, DODMA and DMDMA, DODAC, DLenDMA, DMRIE, CLinDMA, CpLinDMA, DMOBA, DOcarbDAP, DLinDAP, DLincarbDAP, DLinCDAP, KLin-K-DMA, DLin-K-XTC2-DMA, HGT4003, and combinations thereof.

58. A method of treating a subject in need thereof comprising administering a therapeutically effective amount of a composition comprising the circular RNA polynucleotide of any one of claims 1-21, a nanoparticle, and optionally a targeting moiety operably connected to the nanoparticle.

59. A method of treating a subject in need thereof comprising administering a therapeutically effective amount of a composition comprising the RNA preparation of any one of claims 36-40, a nanoparticle, and optionally, a targeting moiety operably connected to a nanoparticle.

60. The method of claim 58 or 59, wherein the targeting moiety is an scFv, nanobody, peptide, minibody, heavy chain variable region, light chain variable region or fragment thereof.

61. The method of any of one of claims 58-60, wherein the nanoparticle is a lipid nanoparticle, a core-shell nanoparticle, or a biodegradable nanoparticle.

62. The method of any one of claims 58-61, wherein the nanoparticle comprises one or more cationic lipids, ionizable lipids, or poly O-amino esters.

63. The method of any one of claims 58-62, wherein the nanoparticle comprises one or more non-cationic lipids.

64. The method of any one of claims 58-63, wherein the nanoparticle comprises one or more PEG-modified lipids, polyglutamic acid lipids, or hyaluronic acid lipids.

65. The method of any one of claims 58-64, wherein the nanoparticle comprises cholesterol.

66. The method of any one of claims 58-65, wherein the nanoparticle comprises arachidonic acid or oleic acid.

67. The method of any one of claims 58-66, wherein the composition comprises a targeting moiety, wherein the targeting moiety mediates receptor-mediated endocytosis into selected cells of a selected cell population in the absence of cell isolation or purification.

68. The method of any one of claims 58-67, wherein the nanoparticle encapsulates more than one circular RNA polynucleotide.

69. A vector for making a circular RNA polynucleotide comprising, in the following order, a 5′ duplex forming region, a 3′ Group I intron fragment, an Internal Ribosome Entry Site (IRES), an expression sequence encoding one or more adjuvants, antigens, adjuvant-like or antigen-like polypeptides, or fragments thereof, a 5′ Group I intron fragment, and a 3′ duplex forming region.

70. A vector for making a circular RNA polynucleotide comprising, in the following order, a 5′ duplex forming region, a 3′ Group I intron fragment, an Internal Ribosome Entry Site (IRES), a noncoding sequence, a 5′ Group I intron fragment, and a 3′ duplex forming region.

71. The vector of claim 69 or 70, comprising a first spacer between the 5′ duplex forming region and the 3′ group I intron fragment, and a second spacer between the 5′ group I intron fragment and the 3′ duplex forming region.

72. The vector of any one of claims 69-71, wherein the first and second spacers each have a length of about 20 to about 60 nucleotides.

73. The vector of any one of claims 69-72, wherein the first and second spacers each comprise an unstructured region at least 5 nucleotides long.

74. The vector of any one of claims 69-73, wherein the first and second spacers each comprise a structured region at least 7 nucleotides long.

75. The vector of any one of claims 69-74, wherein the first and second duplex forming regions each have a length of about 9 to 50 nucleotides.

76. The vector of any one of claims 69-75, wherein the vector is codon optimized.

77. The vector of any one of claims 69-76, lacking at least one microRNA binding site present in an equivalent pre-optimization polynucleotide.

78. A prokaryotic cell comprising a vector of any one of claims 69-77.

79. A eukaryotic cell comprising a circular RNA polynucleotide of any one of claims 1-21.

80. The eukaryotic cell of claim 79, wherein the eukaryotic cell is a human cell.

81. The eukaryotic cell of claim 79 or 80, wherein the eukaryotic cell is an antigen presenting cell.

82. A vaccine, comprising: at least one circular RNA polynucleotide having an expression sequence encoding at least one viral antigenic polypeptide, adjuvant or adjuvant-like polypeptide, or an immunogenic fragment thereof, formulated in a lipid nanoparticle.

83. The vaccine of claim 82, wherein the adjuvant or adjuvant-like polypeptide is selected from Table 10.

84. The vaccine of claim 82 or 83, wherein the antigenic polypeptide is a viral polypeptide from an adenovirus; Herpes simplex, type 1; Herpes simplex, type 2; encephalitis virus, papillomavirus, Varicella-zoster virus; Epstein-barr virus; Human cytomegalovirus; Human herpes virus, type 8; Human papillomavirus; BK virus; JC virus; Smallpox; polio virus; Hepatitis B virus; Human bocavirus; Parvovirus B19; Human astrovirus; Norwalk virus; coxsackievirus; hepatitis A virus; poliovirus; rhinovirus; Severe acute respiratory syndrome virus; Hepatitis C virus; Yellow Fever virus; Dengue virus; West Nile virus; Rubella virus; Hepatitis E virus; Human Immunodeficiency virus (HIV); Influenza virus; Guanarito virus; Junin virus; Lassa virus, Machupo virus; Sabia virus; Crimean-Congo hemorrhagic fever virus, Ebola virus; Marburg virus; Measles virus; Mumps virus; Parainfluenza virus; Respiratory syncytial virus; Human metapneumo virus; Hendra virus; Nipah virus; Rabies virus; Hepatitis D; Rotavirus; Orbivirus; Coltivirus; Banna virus; Human Enterovirus; Hanta virus; West Nile virus; Middle East Respiratory Syndrome Corona Virus; Japanese encephalitis virus; Vesicular exanthernavirus; SARS-CoV-2; Eastern equine encephalitis, or a combination of any two or more of the foregoing.

85. The vaccine of any of claims 82-84, wherein the viral antigenic polypeptide or an immunogenic fragment thereof is selected or derived from any one of SEQ ID NOs: 325-336.

86. The vaccine of any one of claims 82-85, wherein the viral antigenic polypeptide or an immunogenic fragment thereof has an amino acid sequence that has at least 90°/% identity to an amino acid sequence of any one of SEQ ID NOs: 325-336, and wherein the viral antigenic polypeptide or immunogenic fragment thereof has membrane fusion activity, attaches to cell receptors, causes fusion of viral and mammalian cellular membranes, and/or is responsible for binding of the virus to a cell being infected.

87. The vaccine of any one of claims 82-86, wherein the expression sequence is codon-optimized.

88. The vaccine of any one of claims 82-87, wherein the vaccine is multivalent.

89. The vaccine of any one of claims 82-88, formulated in an effective amount to produce an antigen-specific immune response.

90. The vaccine of any one of claims 82-89, wherein the circular RNA polynucleotide comprises a first expression sequence encoding a first viral antigenic polypeptide and a second expression sequence encoding a second viral antigenic polypeptide.

91. A method of inducing an immune response in a subject, the method comprising administering to the subject the vaccine of any one of claims 82-90, in an amount effective to produce an antigen-specific immune response in the subject.

92. The method of claim 91, wherein the antigen-specific immune response comprises a T cell response or a B cell response.

93. The method of claim 91 or 92, wherein the subject is administered a single dose of the vaccine.

94. The method of any one of claims 91-93, wherein the subject is administered a booster dose of the vaccine.

95. The method of any one of claims 91-94, wherein the vaccine is administered to the subject by intranasal administration, intradermal injection or intramuscular injection.

96. The method of any one of claims 91-95, wherein an anti-antigenic polypeptide antibody titer produced in the subject is increased by at least 1 log relative to a pre-determined threshold level.

97. The method of any one of claims 91-96, wherein an anti-antigenic polypeptide antibody titer produced in the subject is increased by 1-3 log relative to a pre-determined threshold level.

98. The method of any one of claims 91-97, wherein the anti-antigenic polypeptide antibody titer produced in the subject is increased at least 2 times relative to a pre-determined threshold level.

99. The method of any one of claims 91-98, wherein the anti-antigenic polypeptide antibody titer produced in the subject is increased 2-10 times relative to a pre-determined threshold level.

100. The method of any one of claims 91-99, wherein the pre-determined threshold level is an anti-antigenic polypeptide antibody titer produced in a subject who has not been administered a vaccine comprising the antigenic polypeptide.

101. The method of any one of claims 91-100, wherein the pre-determined threshold level is an anti-antigenic polypeptide antibody titer produced in a subject who has been administered a live attenuated vaccine or an inactivated vaccine comprising the antigenic polypeptide.

102. The method of any one of claims 91-101, wherein the pre-determined threshold level is an anti-antigenic polypeptide antibody titer produced in a subject who has been administered a recombinant protein vaccine or purified protein vaccine comprising the antigenic polypeptide.

103. A SARS-CoV2 vaccine, comprising: at least one circular RNA polynucleotide having an expression sequence encoding at least one SARS-CoV2 viral antigenic polypeptide or an immunogenic fragment thereof, formulated in a lipid nanoparticle.

104. The SARS-CoV2 vaccine of claim 102, wherein the SARS-CoV2 viral antigenic polypeptide is selected from: SARS-CoV2 spike protein, Nsp1-Nsp16, ORF3a, ORF6, ORF7a, ORFb, ORF8, ORF10, SARS-CoV2 envelope protein, SARS-CoV2 Membrane protein, SARS-CoV2 nucleocapsid protein or any antigenic peptide of SARS-CoV2 or fragment of SARS-CoV2 peptide.

105. The SARS-CoV2 vaccine of claim 102103 or 104, wherein the SARS-CoV2 viral antigenic polypeptide is derived from SARS-CoV2 virus strain G, strain GR, strain GH, strain L, strain V, or a combination thereof.

106. The SARS-CoV2 vaccine of any one of claims 103-105, wherein the expression sequence is codon-optimized.

107. The SARS-CoV2 vaccine of any one of claims 103-106, wherein the vaccine is multivalent.

108. The SARS-CoV2 vaccine of any one of claims 103-107, formulated in an effective amount to produce an antigen-specific immune response.

109. A method of inducing an immune response in a subject, the method comprising administering to the subject the SARS-CoV2 vaccine of any one of claims 103-108, in an amount effective to produce an antigen-specific immune response in the subject.

110. The method of claim 109, wherein the antigen-specific immune response comprises a T cell response or a B cell response.

111. The method of claim 109 or 110, wherein the subject is administered a single dose of the vaccine.

112. The method of claim any one of claims 109-111, wherein the subject is administered a booster dose of the vaccine.

113. The method of any one of claims 109-112, wherein the vaccine is administered to the subject by intranasal administration, intradermal injection or intramuscular injection.

114. The method of anyone of claims 109-113, wherein an anti-antigenic polypeptide antibody titer produced in the subject is increased by at least 1 log relative to a pre-determined threshold level.

115. The method of any one of claims 109-114, wherein an anti-antigenic polypeptide antibody titer produced in the subject is increased by 1-3 log relative to a pre-determined threshold level.

116. The method of any one of claims 109-115, wherein the anti-antigenic polypeptide antibody titer produced in the subject is increased at least 2 times relative to a pre-determined threshold level.

117. The method of anyone of claims 109-116, wherein the anti-antigenic polypeptide antibody titer produced in the subject is increased 2-10 times relative to a pre-determined threshold level.

118. The method of any one of claims 109-117, wherein the pre-determined threshold level is an anti-antigenic polypeptide antibody titer produced in a subject who has not been administered a vaccine comprising the antigenic polypeptide.

119. The method of any one of claims 109-118, wherein the pre-determined threshold level is an anti-antigenic polypeptide antibody titer produced in a subject who has been administered a live attenuated vaccine or an inactivated vaccine comprising the antigenic polypeptide.

120. The method of any one of claims 109-119, wherein the pre-determined threshold level is an anti-antigenic polypeptide antibody titer produced in a subject who has been administered a recombinant protein vaccine or purified protein vaccine comprising the antigenic polypeptide.

121. A circular RNA polynucleotide having an expression sequence encoding at least one viral antigenic polypeptide, adjuvant or adjuvant-like polypeptide, or an immunogenic fragment thereof.

122. An expression vector comprising an engineered nucleic acid encoding at least one circular RNA polynucleotide of any one of claims 1-21.

123. A circular RNA polynucleotide vaccine comprising the circular RNA polynucleotide of claim 121, formulated in a lipid nanoparticle.

124. The circular RNA polynucleotide vaccine of claim 123, wherein the nanoparticle has a mean diameter of 50-200 nm.

125. The circular RNA polynucleotidevaccine of claim 123 or 124, wherein the lipid nanoparticle comprises a cationic lipid, a PEG-modified lipid, a sterol and a non-cationic lipid.

126. The circular RNA polynucleotidevaccine of any one of claims 123-125, wherein the lipid nanoparticle carrier comprises a molar ratio of about 20-60% cationic lipid, 0.5-15% PEG-modified lipid, 25-55% sterol, and 25% non-cationic lipid.

127. The circular RNA polynucleotide vaccine of any one of claims 123-126, wherein the cationic lipid is an ionizable cationic lipid and the non-cationic lipid is a neutral lipid, and the sterol is a cholesterol.

128. The circular RNA polynucleotidevaccine of any one of 123-127, wherein the cationic lipid is selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319).

129. The circular RNA polynucleotide vaccine of any one of claims 123-128, wherein the nanoparticle has a polydispersity value of less than 0.4.

130. The circular RNA polynucleotide vaccine of any one of claims 123-129, wherein the nanoparticle has a net neutral charge at a neutral pH value.

131. A pharmaceutical composition for use in vaccination of a subject, comprising an effective dose of circular RNA polynucleotide encoding at least one viral antigen or adjuvant or adjuvant-like polypeptide, or an immunogenic fragment thereof, wherein the effective dose is sufficient to produce a 1,000-10,000 neutralization titer produced by neutralizing antibody against said antigen or adjuvant or adjuvant-like polypeptide, or an immunogenic fragment thereof, as measured in serum of the subject at 1-72 hours post administration.

132. A pharmaceutical composition for use in vaccination of a subject, comprising an effective dose of circular mRNA encoding at least one viral antigen or adjuvant or adjuvant-like polypeptide, or an immunogenic fragment thereof, wherein the effective dose is sufficient to produce detectable levels of antigen or adjuvant or adjuvant-like polypeptide, or an immunogenic fragment thereof, as measured in serum of the subject at 1-72 hours post administration.

133. A method of inducing, producing, or enhancing an immune response in a subject, the method comprising administering to the subject the pharmaceutical composition of claim 131 or 132, in an amount effective to induce, produce or enhance an antigen-specific immune response in the subject.

134. The method of claim 133, wherein the pharmaceutical composition immunizes the subject against the virus for up to 2 years.

135. The method of claim 133 or 134, wherein the pharmaceutical composition immunizes the subject against the virus for more than 2 years.

136. The method of any one of claims 133-135, wherein the subject has been exposed to the virus, wherein the subject is infected with the virus, or wherein the subject is at risk of infection by the virus.

137. The method of any one of claims 133-136, wherein the subject is immunocompromised.

138. The vaccine of any one of claims 82-90, 103-108, and 123-130, or the pharmaceutical composition of claim 131 or 132, for use in a method of inducing an antigen specific immune response in a subject, the method comprising administering to the subject the vaccine or the pharmaceutical composition in an amount effective to produce an antigen specific immune response in the subject.

139. Use of the vaccine of any one of claims 82-90, 103-108, and 123-130, or the pharmaceutical composition of claim 131 or 132, in the manufacture of a medicament for use in a method of inducing an antigen specific immune response in a subject, the method comprising administering to the subject the vaccine in an amount effective to produce an antigen specific immune response in the subject.

140. A method of inducing cross-reactivity against a variety of viruses or strains of a virus in a mammal, the method comprising administering to the mammal in need thereof the vaccine of any preceding claim or the pharmaceutical composition of any preceding claim.

141. The method of claim 140, wherein at least two circular RNA polynucleotides having an expression sequence each encoding a consensus viral antigen are administered to the mammal separately.

142. The method of claim 140 or 141, wherein at least two circular RNA polynucleotides having an expression sequence each encoding a consensus viral antigen are administered to the mammal simultaneously.

143. The vaccine of any one of claims 82-90, 103-108, and 123-130, wherein the circular RNA polynucleotide is co-formulated with an adjuvant in the same nanoparticle.

144. The vaccine of any one of claims 82-90, 103-108, 123-130, and 143, wherein the adjuvant is CpG, imiquimod, Aluminium, or Freund's adjuvant.


SEQUENCE LISTING (351 sequences)

SEQ ID NO: 1 - DNA, 585 bp, Unknown
  source: /note=“Description of Unknown Encephalomyocarditis virus sequence”
cccccctctccctccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaacc

SEQ ID NO: 2 - DNA, 568 bp, Unknown
  source: /note=“Description of Unknown Encephalomyocarditis virus sequence”
ctccccctccccccccttactatactggccgaagccacttggaataaggccggtgtgcgtttgtctacatgctattttctaccgcattaccgtcttatggtaatgtgagggtccagaacctgaccctgtcttcttgacgaacactcctaggggtctttcccctctcgacaaaggagtgtaaggtctgttgaatgtcgtgaaggaagcagttcctctggaagcttcttaaagacaaacaacgtctgtagcgaccctttgcaggcagcggaaccccccacctggtgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacgtgctttacacgtgttgagtcgaggtgaaaaaacgtctaggccccccgaaccacggggacgtggttttcctttgaaaaccacgattacaat

SEQ ID NO: 3 - DNA, 696 bp, Unknown
  source: /note=“Description of Unknown Encephalomyocarditis virus sequence”
ttgccagtctgctcgatatcgcaggctgggtccgtgactacccactccccctttcaacgtgaaggctacgatagtgccagggcgggtactgccgtaagtgccaccccaaacaacaacaacaaaacaaactccccctccccccccttactatactggccgaagccacttggaataaggccggtgtgcgtttgtctacatgctattttctaccgcattaccgtcttatggtaatgtgagggtccagaacctgaccctgtcttcttgacgaacactcctaggggtctttcccctctcgacaaaggagtgtaaggtctgttgaatgtcgtgaaggaagcagttcctctggaagcttcttaaagacaaacaacgtctgtagcgaccctttgcaggcagcggaaccccccacctggtgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacgtgctttacacgtgttgagtcgaggtgaaaaaacgtctaggccccccgaaccacggggacgtggttttcctttgaaaaccacgattacaat

SEQ ID NO: 4 - DNA, 697 bp, Unknown
  source: /note=“Description of Unknown Encephalomyocarditis virus sequence”
ttgccagtctgctcgatatcgcaggctgggtccgtgactacccactccccctttcaacgtgaaggctacgatagtgccagggcgggtactgccgtaagtgccaccccaaaacaacaacaaccccccctctccctcctccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataat

SEQ ID NO: 5 - DNA, 562 bp, Unknown
  source: /note=“Description of Unknown Encephalomyocarditis virus sequence”
ccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataat

SEQ ID NO: 6 - DNA, 156 bp, Unknown
  source: /note=“Description of Unknown Picobirnavirus sp. sequence”
gtaaattaaatgctatttacaaaatttaaacagaaaggagagatgttatgaaccggttttacaaggtttcatacatcgaaaatagcactacctggggcagccgacacactaacatcgtctgtttaaccagaagtgttactgaaaggaggttattta

SEQ ID NO: 7 - DNA, 340 bp, Unknown
  source: /note=“Description of Unknown Hepatitis C virus sequence”
acctgcccctaataggggcgacactccgccatgaatcactcccctgtgaggaactactgtcttcacgcagaaagcgtctagccatggcgttagtatgagtgtcgtacagcctccaggcccccccctcccgggagagccatagtggtctgcggaaccggtgagtacaccggaattgccgggaagactgggtcctttcttggataaacccactctatgcccggacatttgggcgtgcccccgcaagactgctagccgagtagcgttgggttgcgaaaggccttgtggtactgcctgatagggtgcttgcgagtgccccgggaggtctcgtagaccgtgcatc

SEQ ID NO: 8 - DNA, 315 bp, Unknown
  source: /note=“Description of Unknown Human Cosavirus sequence”
ctacaagctttgtgtaaacaaacttttgtttggcttttctcaagcttctctcacatcaggccccaaagatgtcctgaaggtaccccgtgtatctgaggatgagcaccatcgactacccggacctgcaaaattttgcaaacgcatgtggtatcccagccccctcctctcggggagggggctttgctcactcagcacaggatctgatcaggagatccacctccggtgctttacaccggggcgtggatttaaaaattgcccaaggcctggcgcacaacctaggggactaggttttccttatattttaaagctgtcaat

SEQ ID NO: 9 - DNA, 182 bp, Unknown
  source: /note=“Description of Unknown Human Cosavirus sequence”
gtcttaggacgacgcatgtggtatcccagcccccgcctacattggcgggggcttttgaagcaccagacactggatctgatcaggaggagggtagctgctttacagcccctcttaaaaattgcccaaggtccggccacccaacctaggggactaggttttccttttatttttaaattgtcatt

SEQ ID NO: 10 - DNA, 759 bp, Unknown
  source: /note=“Description of Unknown Human Cosavirus sequence”
acatgggggagactgcatgtggcagtcttgaaacgtgtggtttgacgtctaccttatatggcagtgggtggagtactgcaaagatgtcaccgtgctttacacggtttttgaaccccacaccggctgtttgacgctcgtagggcagcaggtttattttcattaaaattcttactttctagctgcatgagttctattcatgcagacggagtgatactcccgttccttcttggacaggttgcctccacgccctttgtggatcttaaggtgaccaagtcactggtgttggaggtgaagatagagagtcctcttgggaatgtcatgtggctgtgccaggggttgtagcgatgccattcgtgtgtgcggatttcctctcgtggtgacacgagcctcacaggccaaaagccccgtccgaaaggacccgaatggtggagtgaccctgactcccccctgcatagttttgtgattaggaacttgaggaatttctgtcataaatctctatcacatcaggccccaaagatgtcctgaaggtaccctgtgtatctgaggatgagcaccaccgactacccggacttgcattagcagacacatgtggttgcccagccccacctcttcagaggtggggctttgctcactcagcacaggatctgatcaggagccccgctcgtgtgctttacactcgacgcggggttaaaaattgcccaaggcctggcacaacaacctaggggactaggttttcctatttttgtaaattatgtcaat

SEQ ID NO: 11 - DNA, 478 bp, Unknown
  source: /note=“Description of Unknown Human rhinovirus sequence”
gtgacaatcagccagattgttaacggtcaagcacttctgtttccccggtacccttgtatacgcttcacccgaggcgaaaagtgaggttatcgttatccgcaaagtgcctacgagaagcctagtagcacttttgaagcctatggctggtcgctcaactgtttacccagcagtagacctggcagatgaggctagatgttccccaccagcgatggtgatctagcctgcgtggctgcctgcacactctattgagtgtgaagccagaaagtggacaaggtgtgaagagcctattgtgctcactttgagtcctccggcccctgaatgtggctaatcctaaccccgtagctgttgcatgtaatccaacatgtctgcagtcgtaatgggcaactatgggatggaaccaactactttgggtgtccgtgtttcttgtttttctttatgcttgcttatggtgacaactgtagttattacatttgttacc

SEQ ID NO: 12 - DNA, 628 bp, Unknown
  source: /note=“Description of Unknown Human rhinovirus sequence”
ttaaaacagcggatgggtatcccaccattcgacccattgggtgtagtactctggtactatgtacctttgtacgcctgtttctccccaaccacccttccttaaaattcccacccatgaaacgttagaagcttgacattaaagtacaataggtggcgccatatccaatggtgtctatgtacaagcacttctgtttcccaggagcgaggtataggctgtacccactgccaaaagcctttaaccgttatccgccaaccaactacgtaacagttagtaccatcttgttcttgactggacgttcgatcaggtggattttccctccactagtttggtcgatgaggctaggaattccccacgggtgaccgtgtcctagcctgcgtggcggccaacccagcttatgctgggacgcccttttaaggacatggtgtgaagactcgcatgtgcttggttgtgagtcctccggcccctgaatgcggctaaccttaaccctagagccttatgccacgatccagtggttgtaaggtcgtaatgagcaattccgggacgggaccgactactttgggtgtccgtgtttctcatttttcttcatattgtcttatggtcacagcatatatatacatatactgtgatc

SEQ ID NO: 13 - DNA, 618 bp, Unknown
  source: /note=“Description of Unknown Human rhinovirus sequence”
ttaaaactgggagtgggttgttcccactcactccacccatgcggtgttgtactctgttattacggtaactttgtacgccagtttttcccacccttccccataatgtaacttagaagtttgtacaatatgaccaataggtgacaatcatccagactgtcaaaggtcaagcacttctgtttccccggtcaatgaggatatgctttacccaaggcaaaaaccttagagatcgttatccccacactgcctacacagagcccagtaccatttttgatataattgggttggtcgctccctgcaaacccagcagtagacctggcagatgaggctggacattccccactggcgacagtggtccagcctgcgtggctgcctgctcacccttcttgggtgagaagcctaattattgacaaggtgtgaagagccgcgtgtgctcagtgtgcttcctccggcccctgaatgtggctaaccttaaccctgcagccgttgcccataatccaatgggtttgcggtcgtaatgcgtaagtgcgggatgggaccaactactttgggtgtccgtgtttcctgtttttcttttgattgcattttatggtgacaatttatagtgtatagattgtcatc

SEQ ID NO: 14 - DNA, 610 bp, Unknown
  source: /note=“Description of Unknown Human rhinovirus sequence”
ttaaaactgggtacaggttgttcccacctgtatcacccacgtggtgtggtgctcttgtattccggtacacttgcacgccagtttgccacccctcacccgtcgtaacttagaagctaacaactcgaccaacaggcggtggtaaaccataccacttacggtcaagcactcctgtttccccggtatgcgaggaatagactcctacagggttgaagcctcaagtatcgttatccgcattggtactacgcaaagcttagtagtgccttgaaagtcccttggttggtcgctccgctagtttcccctagtagacctggcagatgaggcaggacactccccactggcgacagtggtcctgcctgcgtggctgcctgcgcacccttaggggtgcgaagccaagtgacagacaaggtgtgaagagccccgtgtgctaccaatgagtcctccggcccctgaatgcggctaatccaaccccacagctattgcacacaagccagtgtgtatgtagtcgtaatgagcaattgtgggacggaaccgactactttgggtgtccgtgtttccttttattcttatcattctgcttatggtgacaatactgtgaaatagtgttgttacc

SEQ ID NO: 15 - DNA, 614 bp, Unknown
  source: /note=“Description of Unknown Human rhinovirus sequence”
taaaactggatccaggttgttcccacctggatctcctattgggagttgtactctattattccggtaattttgtacgccagttttatcttccccctccccaattgtaacttagaaggttatcaatacgaccaataggtggtagttagccaaactaccaaaggtcaagcacttctgtttccccggtcaaagttgatatgctccaacagggcaaaaacaactgagatcgttatccgcaaagtgcctacgcaaagcctagtaacacctttgaagatttatggttggtcgttccgctatttcccatagtagacctggcagatgaggctagaaatcccccactggcgacagtgctctagcctgcgtggctgcctgcgcaccccttgggtgcgaagccatacattggacaaggtgtgaagagccccgtgtgctcactttgagtcctccggcccctgaatgtggctaaccttaaccctgcagctagtgcatgtaatccaacatgttgctagtcgtaatgagtaattgcgggacgggaccaactactttgggtgtccgtgtttcactttttccttttaatattgcttatggtgacaatatatatagctatatatattgacacc

SEQ ID NO: 16 - DNA, 624 bp, Unknown
  source: /note=“Description of Unknown Salivirus A sequence”
ttcccctgcaaccattacgcttactcgcatgtgcattgagtggtgcatgtgttgaacaaacagctacactcacatgggggcgggttttcccgccctacggcttctcgcgaggcccacccctcccctttctcccataactacagtgctttggtaggtaagcatcctgatcccccgcggaagctgctcacgtggcaactgtggggacccagacaggttatcaaaggcacccggtctttccgccttcaggagtatccctgctagcgaattctagtagggctctgcttggtgccaacctcccccaaatgcgcgctgcgggagtgctcttccccaactcaccctagtatcctctcatgtgtgtgcttggtcagcatatctgagacgatgttccgctgtcccagaccagtccagtaatggacgggccagtgtgcgtagtcgtcttccggcttgtccggcgcatgtttggtgaaccggtggggtaaggttggtgtgcccaacgcccgtactcaggggatacctcaaggcacccaggaatgccagggaggtaccccgcttcacagcgggatctgaccctggggtaaatgtctgcggggggtcttcttggcccacttctcagtacttttcagg

SEQ ID NO: 17 - DNA, 730 bp, Unknown
  source: /note=“Description of Unknown Salivirus FHB sequence”
acatggggggtctgcggacggcttcggcccacccgcgacaagaatgccgtcatctgtcctcattacccgtattccttcccttcccccgcaaccaccacgcttactcgcgcacgtgttgagtggcacgtgcgttgtccaaacagctacacccacacccttcggggcgggtttgtcccgccctcgggttcctcgcggaacccccccctccctctctctctttctatccgccctcacttcccataactacagtgctttggtaggtgagcaccctgaccccccgcggaagctgctaacgtggcaactgtggggatccaggcaggttatcaaaggcacccggtctttccgccttcaggagtatctctgccggtgaattccggtagggctctgcttggtgccaacctcccccaaatgcgcgctgcgggagtgctcttccccaactcatcttagtaacctctcatgtgtgtgcttggtcagcatatctgaggcgacgttccgctgtcccagaccagtccagcaatggacgggccagtgtgcgtagtcgctttccggttttccggcgcatgtttggcgaaacgctgaggtaaggttggtgtgcccaacgcccgtaatttggtgatacctcaagaccacccaggaatgccagggaggtaccccacttcggtgggatctgaccctgggctaattgtctacggtggttcttcttgcttccacttctcttttttctggcatg

SEQ ID NO: 18 - DNA, 709 bp, Unknown
  source: /note=“Description of Unknown Salivirus NG-J1 sequence”
tatggcaggcgggcttgtggacggcttcggcccacccacagcaagaatgccatcatctgtcctcacccccaattttcccttttcttcccctgcaaccattacgcttactcgcatgtgcattgagtggtgcatgtgttgaacaaacagctacactcacatgggggcgggttttcccgccctacggcctctcgcgaggcccaccccttccctccccttataactacagtgctttggtaggtaagcatcctgatcccccgcggaagctgctcacgtggcaactgtggggacccagacaggttatcaaaggcacccggtctttccgccttcaggagtatccctactagtgaattctagcggggctctgcttggtgccaacctcccccaaatgcgcgctgcgggagtgctcttccccaactcaccctagtatcctctcatgtgtgtgcttggtcagcatatctgagacgatgttccgctgtcccagaccagtccagtaatggacgggccagtgcgtgtagtcgtcttccggcttgtccggggcatgtttggtgaaccggtggggtaaggttggtgtgcccaacgcccgtactttggtgacacctcaagaccacccaggaatgccagggaggtaccccacctcacggtgggatctgaccctgggctaattgtctacggtggttcttcttgcttccacttctttcttctgttcacg

SEQ ID NO: 19 - DNA, 708 bp, Unknown
  source: /note=“Description of Unknown Human Parechovirus sequence”
tttgaaaggggtctcctagagagcttggccgtcgggccttataccccgacttgctgagtttctctaggagagcccttttcccagccctgaggcggctggtcaataaaagcctcaaacgtaactaacacctaagaagatcatgtaaaccctatgcctggtctccactattcgaaggcaacttgcaataagaagagtgggatcaagacgcttaaagcatagagacagttttcttttctaacccacatttgtgtggggtggcagatggcgtgccataactctaatagtgagataccacgcttgtggaccttatgctcacacagccatcctctagtaagtttgtgagacgtctggtgacgtgtgggaacttattggaaacaacattttgctgcaaagcatcctactgccagcggaaaaacacctggtaacaggtgcctctggggccaaaagccaaggtttaacagaccctttaggattggttctaaacctgagatgttgtggaagatatttagtacctgctgatctggtagttatgcaaacactagttgtaaggcccatgaaggatgcccagaaggtacccgtaggtaacaagtgacactatggatctgatttggggccagatacctctatcttggtgatctggttaaaaaacatctaatgggccaaacccgggggggatccccggtttcctcttattctatcaatgccact

SEQ ID NO: 20 - DNA, 508 bp, Unknown
  source: /note=“Description of Unknown Crohivirus B sequence”
gtataagagacaggtgtttgccttgtcttcggactggcatcttgggaccaaccccccttttccccagccatgggttaaatggcaataaaggacgtaacaactttgtaaccattaagctttgtaattttgtaaccactaagctttgtgcacataatgtaaccatcaagcttgttagtcccagcaggaggtttgcatgcttgtagccgaaatggggctcgaccccccatagtaggatacttgattttgcattccattgtggacctgcaaactctacacatagaggctttgtcttgcatctaaacacctgagtacagtgtgtacctagaccctatagtacgggaggaccgtttgtttcctcaataaccctacataataggctaggtgggcatgcccaatttgcaagatcccagactgggggtcggtctgggcagggttagatccctgttagctactgcctgatagggtggtgctcaaccatgtgtagtttaaattgagctgttcatatacc

SEQ ID NO: 21 - DNA, 248 bp, Unknown
  source: /note=“Description of Unknown Picornaviridae sp. sequence”
actgaagatcctacagtaactactgccccaatgaacgccacagatgggtctgctgatgactacctatcttagtgctagttgaggtttgaagtgagccggtttttagaagaaccagtttctgaacattatcatccccagcatctattctatacgcacaagatagatagtcatcagcagacacatctgtgctactgcttgatagagttgcggctggtcaacttagattggtataaccagttgagtggcaa

SEQ ID NO: 22 - DNA, 516 bp, Unknown
  source: /note=“Description of Unknown Rosavirus M-7 sequence”
tatgcatcactggacggcctaacctcggtcgtggcttcttgccgatttcagcgctaccaggctttctggtctcgccaggcgttgattagtaggtgcactgtctaagtgaagacagcagtgctctctgtgaaaagttgatgacactcttcaggtttgtagcgatcactcaaggctagcggatttccccgtgtggtaacacacgcctctaggcccagaaggcacggtgttgacagcaccccttgagtggctggtcttccccaccagcacctgatttgtggattcttcctagtaacggacaagcatggctgctcttaagcattcagtgcgtccggggctgaaggatgcccagaaggtacccgcaggtaacgataagctcactgtggatctgatctggggctgcgggctgggtgtctttccacccagccaaaacccgtaaaacggtagtcgcagttaaaaaacgtctaggccccacccccccagggatggggggttcccttaaaccctcacaagttcaac

SEQ ID NO: 23 - DNA, 478 bp, Unknown
  source: /note=“Description of Unknown Shanbavirus A sequence”
tgaaaagggggcgcagggtggtggtggttactaaatacccaccatcgccctgcacttcccttttcccctgtggctcagggtcacttagccccctctttgggttaccagtagttttctacccctgggcacagggttaactatgcaagacggaacaacaatctcttagtccccctcgccgatagtgggctcgacccccatgtgtaggagtggataagggacggagtgagccgatacggggaagagtgtgcggtcacaccttaattccatgagcgctgcgaagaaggaagctgtgaacaatggcgacctgaaccgtacacatggagctccacaggcatggtactcgttagactacgcagcctggttgggagtgggtataccctgggtgagccgccagtgaatgggagttcactggttaacacacactgcctgatagggtcagggcctcctgtccccgccgtaatgaggtagaccatatgcc

SEQ ID NO: 24 - DNA, 403 bp, Unknown
  source: /note=“Description of Unknown Pasivirus A sequence”
gcggctggatattctggccgtgcaactgcttttgaccagtggctctgggtaacttagccaaagtgtccttctccctttccctattatatgttttatggctttgtctggtcttgtttagtttatatataagatcctttccgccgatatagacctcgacagtctagtgtaggaggattggtgatattaatttgccccagaagagtgaccgtgacacatagaaaccatgagtacatgtgtatccgtggaggatcgcccgggactggattccatatcccattgccatcccaacaagcggagggtatacccactatgtgcacgtctgcagtgggagtctgcagatttagtcatactgcctgatagggtgtgggcctgcactctggggtactcaggctgtttatataat

SEQ ID NO: 25 - DNA, 404 bp, Unknown
  source: /note=“Description of Unknown Pasivirus A sequence”
gctggactttctggctgcgcaactgcttttaaccagtggctctgggttacttagccaaaaccccctttccccgtaccctagtttgtgtgtgtattattattttgttgttgttttgtaaatttttatataagatcctttccgccgatatagacctcgacagtctagtgtaggaggattggtgatattaatatgccccagaagagtgaccgtgacacatagaaaccatgagtacatgtgtatccgtggaggatcgcccgggactggattccatatcccattgccatcccaacaaacggagggtatacccgctatgtgcgcgtctacagtgggaatctgtagatttagtcatactgcctgatagggtgtgggcctgcactctggggtactcaggctgtttatataat

SEQ ID NO: 26 - DNA, 744 bp, Unknown
  source: /note=“Description of Unknown Echovirus E14 sequence”
ttaaaacagcctgtgggttgttcccatccacagggcccactgggcgccagcactctggtattgcggtaccttagtgcgcctgttttatatacccgtcccccaaacgtaacttagacgcatgtcaacgaagaccaatagtaagcgcagcacaccagctgtgttccggtcaagcacttctgttaccccggaccgagtatcaataagctactcacgtggctgaaggagaaaacgttcgttacccgaccaattacttcaagaaacctagtaacaccatgaaggttgcgcagtgtttcgctccgcacaaccccagtgtagatcaggtcgatgagtcaccgcattccccacgggtgaccgtggcggtggctgcgctggcggcctgcccatggggaaacccatgggacgcttcaatactgacatggtgcgaagagtctattgagctaattggtagtcctccggcccctgaatgcggctaatcctaactgcggagcagatacccacacaccagtgggcagtctgtcgtaacgggcaactctgcagcggaaccgactactttgggtgtccgtgtttctctttatccttatactggctgcttatggtgacaattgagagattgttaccatatagctattggattggccatccggtgacaaatagagcaattgtgtatttgtttgttggtttcgtgccattaaattacaaggttctaaacacccttaatcttattatagcattcaacacaacaaa

SEQ ID NO: 27 - DNA, 663 bp, Unknown
  source: /note=“Description of Unknown Human Parechovirus sequence”
gtacattagatgcgtcatctgcaactttagtcaataaattacctccaatgtcattaccaacattccctaccttttcactaacacctaagacaacaagtacctatgcctggtctccactattcgaaggcaacttgcaataagaagagtggaattaagacgcttaaagcatagagctagttatcttttctaacccacaaagttttgtggggtggcagatggcgtgccataactctattagtgagataccatgcttgtggatcttatgctcacacagccatcctctagtaagttgataaggtgtctggtgatatgtgggaactcacatgaaccattaatttaccgtaaggtatcctatagccagcggaatcacatctggtgacagatgcctctggggccgaaagccaaggtttaacagaccctataggattggtttcaaaacctgaattgatgtggattgtgtatagtacctgttgatctggtaacagtgtcaacactagttgtaaggcccacgaaggatgcccagaaggtacccgtaggtaacaagtgacactatggatctgatctggggccagctacctctatcatggtgagttggttaaaaaacgtctagtgggccaaacccaggggggatccctggtttccttttacctaatcaaagccact

SEQ ID NO: 28 - DNA, 744 bp, Unknown
  source: /note=“Description of Unknown Aichi virus sequence”
tttgaaaagggggtgggggggcctcggccccctcaccctcttttccggtggtctggtcccggaccaccgttactccattcagcttcttcggaacctgttcggaggaattaaacgggcacccatactccccccaccccccttttgtaactaagtatgtgtgctcgtgatcttgactcccacggaacggaccgatccgttggtgaacaaacagctaggtccacatcctcccttcccctgggagggcccccgccctcccacatcctccccccagcctgacgtatcacaggctgtgtgaagcccccgcgaaagctgctcacgtggcaattgtgggtccccccttcatcaagacaccaggtctttcctccttaaggctagccccggcgtgtgaattcacgttgggcaactagtggtgtcactgtgcgctcccaatctcggccgcggagtgctgttccccaagccaaacccctggcccttcactatgtgcctggcaagcatatctgagaaggtgttccgctgtggctgccaacctggtgacaggtgccccagtgtgcgtaaccttcttccgtctccggacggtagtgattggttaagatttggtgtaaggttcatgtgccaacgccctgtgcgggatgaaacctctactgccctaggaatgccaggcaggtaccccacctccgggtgggatctgagcctgggctaattgtctacgggtagtttcatttccaatccttttatgtcggagtc

SEQ ID NO: 29 - DNA, 734 bp, Unknown
  source: /note=“Description of Unknown Hepatitis A virus sequence”
ttcaagaggggtctccggagttttccggaacccctcttggaagtccatggtgaggggacttgatacctcaccgccgtttgcctaggctataggctaaatttccctttccctgtccttcccctatttccttttgttttgtttgtaaatattaattcctgcaggttcagggttctttaatctgtttctctataagaacactcaatttttcacgctttctgtctcctttcttccagggctctccccttgccctaggctctggccgttgcgcccggcggggtcaactccatgattagcatggagctgtaggagtctaaattggggacgcagatgtttgggacgtcgccttgcagtgttaacttggctttcatgaacctctttgatcttccacaaggggtaggctacgggtgaaacctcttaggctaatacttcaatgaagagatgccttggatagggtaacagcggcggatattggtgagttgttaagacaaaaaccattcaacgccggaggactggctctcatccagtggatgcattgagggaattgattgtcagggctgtctctaggtttaatctcagacctctctgtgcttagggcaaacactatttggccttaaatgggatcctgtgagagggggtccctccattgacagctggactgttctttggggccttatgtggtgtttgcctctgaggtactcaggggcatttaggtttttcctcattcttaaataata

SEQ ID NO: 30 - DNA, 638 bp, Unknown
  source: /note=“Description of Unknown Phopivirus sequence”
gggagtaaacctcaccaccgtttgccgtggtttacggctacctatttttggatgtaaatattaattcctgcaggttcaggtctcttgaattatgtccacgctagtggcactctcttacccataagtgacgccttagcggaacctttctacacttgatgtggttaggggttacattatttccctgggccttctttggccctttttcccctgcactatcattctttcttccgggctctcagcatgccaatgttccgaccggtgcgcccgccggggttaactccatggttagcatggagctgtaggccctaaaagtgctgacactggaactggactattgaagcatacactgttaactgaaacatgtaactccaatcgatcttctacaaggggtaggctacgggtgaaaccccttaggttaatactcatattgagagatacttctgataggttaaggttgctggataatggtgagtttaacgacaaaaaccattcaacagctgtgggccaacctcatcaggtagatgcttttggagccaagtgcgtaggggtgtgtgtggaaatgcttcagtggaaggtgccctcccgaaaggtcgtaggggtaatcaggggcagttaggtttccacaattacaatttgaa

SEQ ID NO: 31 - DNA, 743 bp, Unknown
  source: /note=“Description of Unknown CVA10 sequence”
gctcttccgatctgggttgttcccacccacagggcccactgggcgccagcactctgattccacggaatctttgtgcgcctgttttacaacccttcccaatttgtaacgtagaagcaatacacactactgatcaatagtaggcatggcgcgccagtcatgtcatgatcaagcacttctgttcccccggactgagtatcaatagactgctcacgcggttgaaggagaaaacgttcgttacccggctaactacttcgagaaacctagtagcaccatggaagctgcggagtgtttcgctcagcactttccccgtgtagatcaggtcgatgagtcactgcaatccccacgggcgaccgtggcagtggctgcgttggcggcctgcctatggggcaacccataggacgctctaatgtggacatggtgcgaagagtctattgagctagttagtagtcctccggcccctgaatgcggctaatcctaactgcggagcacatgccttcaacccaggaggtggtgtgtcgtaacgggtaactctgcagcggaaccgactactttgggtgtccgtgtttccttttatccttatattggctgcttatggtgacaatcacggaattgttgccatatagctattggattggccatccggtgtctaacagagctattgtatacctatttgttggatttactcccctatcatacaaatctctgaacactttgtgctttatactgaacttaaacacacgaaa

SEQ ID NO: 32 - DNA, 746 bp, Unknown
  source: /note=“Description of Unknown Enterovirus C sequence”
ttaaaacagctctggggttgttcccaccccagaggcccacgtggcggccagtacaccggtaccacggtacccttgtacgcctgttttatactcccctccccgtaaactagaagcacgaaacacaagttcaatagaagggggtacagaccagtaccaccacgaacaagcacttctgttcccccggtgaggtcacatagactgtccccacggtcaaaagtgactgatccgttatccgctcacgtacttcggaaagcctagtaccaccttggaatctacgatgcgttgcgctcagcactcgaccccggagtgtagcttaggctgatgagtctggacgttccccactggtgacagtggtccaggctgcgttggcggcctacctgtggtccaaaaccacaggacgctagtagtgaacaaggtgtgaagagcccactgagctacctgagaatcctccggcccctgaatgcggctaatcccaaccacggagcaggtaatcgcaaaccagcggtcagcctgtcgtaacgcgtaagtctgtggcggaaccgactactttgggtgtccgtgtttccttttatttttatggtggctgcttatggtgacaatcatagattgttatcataaagcaaattggattggccatccggagtgagctaaactatctatttctctgagtgttggattcgtttcacccacattctgaacaatcagcctcattagtgttaccctgttaataagacgatatcatcacg

SEQ ID NO: 33 - DNA, 726 bp, Unknown
  source: /note=“Description of Unknown Enterovirus D sequence”
ttaaaacagctctggggttgttcccaccccagaggcccacgtggcggctagtactccggtaccccggtacccttgtacgcctgttttatactccctttcccaagtaactttagaagaaataaactaatgttcaacaggagggggtacaaaccagtaccaccacgaacacacacttctgtttccccggtgaagttgcatagactgtacccacggttgaaagcgatgaatccgttacccgcttaggtacttcgagaagcctagtatcatcttggaatcttcgatgcgttgcgatcagcactctaccccgagtgtagcttgggtcgatgagtctggacaccccacaccggcgacgtggtccaggctgcgttggcggcctacccatggctagcaccatgggacgctagttgtgaacaaggtgcgaagagcctattgagctacctgagagtcctccggcccctgaatgcggctaatcccaaccacggagcaaatgctcacaatccagtgagtggtttgtcgtaatgcgcaagtctgtggcggaaccgactactttgggtgtccgtgtttccttttatttttattatggctgcttatggtgacaatctgagattgttatcatatagctattggattagccatccggtgatatcttgaaattttgccataactttttcacaaatcctacaacattacactacactttctcttgaataattgagacaactcata

SEQ ID NO: 34 - DNA, 737 bp, Unknown
  source: /note=“Description of Unknown Enterovirus J sequence”
ttaaaatagcctcagggttgttcccaccctgagggcccacgtggtgtagtactctggtattacggtacctttgtacgcctattttatacccccttccccaagtaatttagaagcaagcacaaaccagttcagtagtaagcagtacaatccagtactgtaatgaacaagtacttctgttaccccggaagggtctatcggtaagctgtacccacggctgaagaatgacctaccgttaaccggctacctacttcgagaagcctagtaatgccgttgaagttttattgacgttacgctcagcacactaccccgtgtgtagttttggctgatgagtcacggcactccccacgggcgaccgtggccgtggctgcgttggcggccaaccaaggagtgcaagctccttggacgtcatattacagacatggtgtgaagagcctattgagctaggtggtagtcctccggcccctgaatgcggctaatcctaactccggagcatatcggtgcgaaccagcacttggtgtgttgtaatacgtaagtctggagcggaaccgactactttgggtgtccgtgtttcctgttttaacttttatggctgcttatggtgacaatttaacattgttaccatatagctgttgggttggccatccggattttgttataaaaccatttcctcgtgccttgacctttaacacatttgtgaacttctttaaatcccttttattagtccttaaatactaaga

SEQ ID NO: 35 - DNA, 327 bp, Pegivirus sp.
aactgttgttgtagcaatgcgcatattgctacttcggtacgcctaattggtaggcgcccggccgaccggccccgcaagggcctagtaggacgtgtgacaatgccatgagggatcatgacactggggtgagcggaggcagcaccgaagtcgggtgaactcgactcccagtgcgaccacctggcttggtcgttcatggagggcatgcccacgggaacgctgatcgtgcaaagggatgggtccctgcactggtgccatgcgcggcaccactccgtacagcctgatagggtggcggcgggcccccccagtgtgacgtccgtggagcgcaac

SEQ ID NO: 36 - DNA, 281 bp, Unknown
  source: /note=“Description of Unknown GB virus C sequence”
tgacgtgggggggttgattttccccccccggcactgggtgcaagccccagaaaccgacgcctatctaagtagacgcaatgactcggcgccgactcggcgaccggccaaaaggtggtggatgggtgatgacagggttggtaggtcgtaaatcccggtcatcctggtagccactataggtgggtcttaagagaaggtcaagattcctcttacgcctgcggcgagaccgcgcacggtccacaggtgttggccctaccggtgtgaataagggcccgacatcaggc

SEQ ID NO: 37 - DNA, 459 bp, Unknown
  source: /note=“Description of Unknown GB virus C sequence”
gacgtgggggggttgatcccccccctttggcactgggtgcaagccccagaaaccgacgcctatttaaacagacgttaagaaccggcgccgacccggcgaccggccaaaaggtggtggatgggtgatgccagggttggtaggtcgtaaatcccggtcatcttggtagccactataggtgggtcttaagggttggttaaggtccctctggcgcttgtggcgagaaagcgcacggtccacaggtgttggccctaccggtgtgaataagggcccgacgtcaggctcgtcgttaaaccgagcccactacccacctgggcaaacaacgcccacgtacggtccacgtcgcccttcaatgtctctcttgaccaataggcttagccggcgagttgacaaggaccagtgggggctgggcggtaggggaaggacccctgccgctgcccttcccggtggagtgggaaatgc

SEQ ID NO: 38 - DNA, 350 bp, Unknown
  source: /note=“Description of Unknown GB virus C sequence”
tgacgtgggggggttgatccgccccccccggcactcggtgcaagccccataaaccgacgcctatctaagtagacgcaatgactcggcgccgactcggcgaccggccaaaaggtggtggatgggtggtgacagggttggtaggtcgtaaatcccggtcatcctggtagccactataggtgggtcttaagagaaggtcaagactcctcttgtgcctgcggcgagaccgcgcacggtccacaggtgctggccctaccggtgtgaataagggcccgacgtcaggctcgtcgttaaaccgagcccgtcacccacctgggcaaacgacgcccacgtacggtccacgtcgcccttca

SEQ ID NO: 39 - DNA, 318 bp, Pegivirus A
tgtagcaatgcgcatattgctacttcggtacgcctaattggtaggcgcccggccgaccggccccgcaagggcctagtaggacgtgtgacaatgccatgcgggatcatgacactggggtgagcggaggcagcaccgaagtcgggtgaactcgactcccagtgcgaccacctggcttggtcgttcatggagggcatgcccacgggaacgctgatcgtgcaaagggatgggtccctgcactggtgccatgcgcggcaccactccgtacagcctgatagggtggcggcgggcccccccagtgtgacgtccgtggagcgcaac

SEQ ID NO: 40 - DNA, 399 bp, Unknown
  source: /note=“Description of Unknown Pasivirus A sequence”
attttctggccgtgtagctgcttttgaccagtggctctgggttacttagccaaatcccccttccttcacccttttaaatttgatggtctgtgttgtttgttttgtcttgtctaaataatatataagatccttcccgccgatacagacctcgacagtctggtgtaggagggttggtgttattaatttgccccagaagagtgaccgtgacacatagaaaccatgagtacatgtgtatccgtggaggatcgcccgggactggattccatatcccattgccatcccaacaagcggagggtatacccactatgtgcgcgtttgcagtgggaatctgcaaatttagtcatactgcctgatagggtgtgggcctgcactctggggtactcaggctgttcatataat

SEQ ID NO: 41 - DNA, 430 bp, Sapelovirus sp.
cccctccacccttaaggtggttgtatcccacataccccaccctcccttccaaagtggacggacaactggattttgactaacggcaagtctgaatggtatgatttggatacgtttaaacggcagtagcgtggcgagctatggaaaaatcgcaattgtcgatagccatgttagtgacgcgcttcggcgtgctcctttggtgattcggcgactggttacaggagagtaggcagtgagctatgggcaaacctctacagtattacttagagggaatgtgcaattgagacttgacgagcgtctctttgagatgtggcgcatgctcttggcattaccatagtgagcttccaggttgggaaacctggactgggcctatactacctgatagggtcgcggctggccgcctgtaactagtatagtcagttgaaaccccccc

SEQ ID NO: 42 - DNA, 504 bp, Rosavirus B
gtctctttagtgtctatgcttcagagagcggtgaactgacaccgttgcttcttgcacagcccttcgtgccggtctttccggttctcgacagcgttgggcatcatggctagttaggctaagatagtggatgatctagtgaacagttttggattgtttggagttttgtagcgatgctagtagtgtgtgtggacctccccacgtggtaacacgtgccccacaggccaaaagccaaggtgttgaaagcacccctactagtcccagactcacccatctgggaactcctctcatgaaaaatcttagtaacttttgattcggctattcatcaacctctctagtcaagggctgaaggatgcccggaaggtacccgcaggtaacgataagctcactgtggatctgatccggggctttggtgcgaccgtctgtccggcgtagccagagttaaaaaacgtctaggcccttccaccccaagggattggggtttccccaatcatttgaaagttcact

SEQ ID NO: 43 - DNA, 532 bp, Unknown
  source: /note=“Description of Unknown Bakunsa virus sequence”
ttttgaacgccacctcggagcgatatccggggaccccctcccctttttccttcctaccttcttcccaaatttccctcttcccttgttattttggtttggatttcctggacatgactcggacggatctatctcatttgctttgtgtctgctccaccagtggcatggtcgaaagatcatcaacactggacgtgtactgtaatggccaaacgtgcccacaggggaaaccatgccggtcgctgtagcggcgggtggacgtggtggacccctctccctgctcataaactttgggtaggtgaagggttcaagcgacgcttgccgtgagggcgcatccggatggtgggaaccaacaaactaggctgtaatggccgacctcaggtggatgagctagggctgctgcaccaaaagggactcgattcgatatcccggcctggtagcctagtgcagtggactcgtagttgggaatctacgactggcctagtacagggtgatagccccgtttcccacgcccacctgttgtagggacacccccccc

SEQ ID NO: 44 - DNA, 494 bp, Tremovirus A
tttgaaagaggcctccggagtgtccggaggctctctttcgacccaacccatactggggggtgtgtgggaccgtacctggagtgcacggtatatatgcattcccgcatggcaagggcgtgctaccttgccccttgacgcatggtatgcgtcatcatttgccttggttaagccccatagaaacgaggcgtcacgtgccgaaaatccctttgcgtttcacagaaccatcctaaccatgggtgtagtatgggaatcgtgtatggggatgattaggatctctcgtagagggataggtgtgccattcaaatccagggagtactctggctctgacattgggacatttgatgtaaccggacctggttcagtatccgggttgtcctgtattgttacggtgtatccgtcttggcacactgaaagggtatttttgggtaatcctttcctactgcctgatagggtggcgtgcccggccacgagagattaagggtagcaatttaaac

SEQ ID NO: 45 - DNA, 378 bp, Unknown
  source: /note=“Description of Unknown Swine Pasivirus 1 sequence”
gcttttgaccagtggctctgggttacttagccaagtccctttctcttattttcactagtttatgttgtgtgttgtctgttttgttttgtttaaattgtatacaagatccttcccgccgacacagacctcgacagtctggtgtaggagggttggtgatattaatttgccccaaaagagtgaccgtgatacgtggaaaccatgagtacatgtgtatccgtggaggatcgcccgggactggattccatatcccattgccatcccaacaaacggagggtatacccaccacgtgcgcgtttgcagtgggaatctgcaaatttagtcatactgcctgatagggtgtgggcctgcactttggggtactcaggctgttcatataat

SEQ ID NO: 46 - DNA, 319 bp, Unknown
  source: /note=“Description of Unknown Parechovirus-like virus PLV-CHN sequence”
acatggggtatgttgtctgtcctgttttgttgaaacaatatataagatcctttccgccgatatagacctcgacagtctagtgtaggaggattggtgatagtaacttgccccagaagagtgaccgtgacacatagaaaccatgagtacatgtgtatccgtggaggatcgcccgggactggattccatatcccattgccatcccaacaaacggagggtatacccactatgtgcgcgtttgcagtgggagcctgcaaatttagtcatactgcctgatagggtgtgggcctgcactctggggtactcaggctgtttatataat

SEQ ID NO: 47 - DNA, 420 bp, Unknown
  source: /note=“Description of Unknown Pasivirus A sequence”
tgaaaaagtggttgtgcagctggattttccggctgtgcaactgcttttgaccagtggctctgggttacttagccaaattcctttcccttatccctattggtttgtgttgtgtgttgtttgttttgttttgtcttaactatatacaagatccttcccgccgatacagacctcgacagtctggtgtaggagggttggtgttattaatttgccccaaaagagtgaccgtgacacgtggaaaccatgagtacatgtgtatccgtggaggatcgcccgggactggattccatatcccattgccatcccaacaaacggagggtatacccaccacgtgcgcgtttgcagtgggaatctgcaaatttagtcatactgcctgatagggtgtgggcctgcactttggggtactcaggctgtttatataat

SEQ ID NO: 48 - DNA, 373 bp, Sicinivirus sp.
gtgtcattaaggtgtgtttggaagttcgaattagctggtttgtggtgattagtagaccccctggaggtacccaattcggatctgaccagggacccgtgactataccgctccggtaattcgggtttaaaacaatgaacgtcaccacacaattacttttctcattttattttcatcattgtcttcctatttaccgattacactcgatttccttggatgttcctggagatttccctggttacctggaccctcattattgttgttgtttcacccagcgagctgtcccaattgcttattatttgcgcttacaacttcgtcctaatatttttctggttgatcgggttgattgagctcccgggctatcctgccattcaac

SEQ ID NO: 49 - DNA, 280 bp, Hepacivirus K
gggaacaatggtccgtccgcggaacgactctagccatgagtctagtacgagtgcgtgccacccattagcacaaaaaccactgactgagccacacccctcccggaatcctgagtacaggacattcgctcggacgacgcatgagcctccatgccgagaaaattgggtatacccacgggtaaggggtggccacccagcgggaatctgggggctggtcactgactatggtacagcctgatagggtgctgccgcagcgtcagtggtatgcggctgttcatggaac

SEQ ID NO: 50 - DNA, 384 bp, Hepacivirus A
acctccgtgctaggcacggtgcgttgtcagcgttttgcgcttgcatgcgctacacgcgtcgtccaacgcggagggaacttcacatcaccatgtgtcactccccctatggagggttccaccccgcttacacggaaatgggttaaccatacccaaagtacgggtatgcgggtcctcctagggcccccccggcaggtcgagggagctggaattcgtgaattcgtgagtacacgaaaatcgcggcttgaacgtctttgaccttcggagccgaaatttgggcgtgccccacgaaggaaggcgggggcggtgttgggccgccgccccctttatcccacggtctgataggatgcttgcgagggcacctgccggtctcgtagaccataggac

SEQ ID NO: 51 - DNA, 384 bp, Unknown
  source: /note=“Description of Unknown Bovine viral diarrhea virus 1 sequence”
gtatacgagaatttgcctaggacctcgtttacaatatgggcaatctaaaattataattaggcctaagggacaaatcctcctcagcgaaggccgaaaagaggctagccatgcccttagtaggactagcaaaataaggggggtagcaacagtggtgagttcgttggatggctgaagccctgagtacagggtagtcgtcagtggttcgacgcttcggaggacaagcctcgagataccacgtggacgagggcatgcccacagcacatcttaacctggacgggggtcgttcaggtgaaaacggtttaaccaaccgctacgaatacagcctgatagggtgctgcagaggcccactgtattgctactgaaaatctctgctgtacatggcac

SEQ ID NO: 52 - DNA, 372 bp, Unknown
  source: /note=“Description of Unknown Border Disease virus sequence”
gtatacgggagtagctcatgcccgtatacaaaattggatattccaaaactcgattgggttagggagccctcctagcgacggccgaaccgtgttaaccatacacgtagtaggactagcagacgggaggactagccatcgtggtgagatccctgagcagtctaaatcctgagtacaggatagtcgtcagtagttcaacgcaggcacggttctgccttgagatgctacgtggacgagggcatgcccaagacttgctttaatctcggcgggggtcgccgaggtgaaaacacctaacggtgttggggttacagcctgatagggtgctgcagaggcccacgaataggctagtataaaaatctctgctgtacatggcac

SEQ ID NO: 53 - DNA, 388 bp, Unknown
  source: /note=“Description of Unknown Bovine viral diarrhea virus 2 sequence”
gtatacgagattagctaaagtactcgtatatggattggacgtcaacaaatttttaattggcaacgtagggaaccttcccctcagcgaaggccgaaaagaggctagccatgccctttagtaggactagcaaaagtagggggactagcggtagcagtgagttcgttggatggccgaacccctgagtacaggggagtcgtcaatggttcgacactccattagtcgaggagtctcgagatgccatgtggacgagggcatgcccacggcacatcttaacccatgcgggggttgcatgggtgaaagcgctaatcgtggcgttatggacacagcctgatagggtgtagcagagacctgctattccgctagtaaaaaactctgctgtacatggcac

SEQ ID NO: 54 - DNA, 373 bp, Unknown
  source: /note=“Description of Unknown Classical swine fever virus sequence”
gtatacgaggttagttcattctcgtatgcattattggacaaatcaaaatttcaatttggttcagggcctccctccagcgacggccgaactgggctagccatgcccatagtaggactagcaaacggagggactagccgtagtggcgagctccctgggtgttctaagtcctgagtacaggacagtcgtcagtagttcgacgtgagcagaagcccacctcgagatgctatgtggacgagggcatgcccaagacgcaccttaaccctagcgggggtcgctagggtgaaatcacaccacgtgatgggagtccgacctgatagggtgctgcagaggctcactattaggctagtataaaaatctctgctgtacatggcac

SEQ ID NO: 55 - DNA, 191 bp, Unknown
  source: /note=“Description of Unknown Human blood-associated dicistrovirus sequence”
aaaaccgaccccagagatcagaaagtcgttgacgcgatcttttattagaggacgttgcgctggcgcgagctttaattagcagacgccaaaaataaacaacaaaatgctgatcgcgagacttaattgtcagacgattggccaaatccgatgtgatctttgctgctcccagattgccgaaataggagtagtag

SEQ ID NO: 56 - DNA, 438 bp, Unknown
  source: /note=“Description of Unknown Hubei Picorna-like virus sequence”
ccccaaaaccccccccttaaactcaacactgtagtggattcattttccgttgcaaaacaaaacattactacccgcatttatgtaggctctgtgttttctatgcgaccgttacattaatctctactctgacccactagtttataaaaccgaagacctgaatgaaacgattttccttcttttcaacctctaacgaacctctgacggcttgagaaacctgaagttagtaattatgtttaaaagaaaggaaagtcaaacgcgatgactcttacatccctattccataccgttgctccacaatgtgagcgatgcgaggtcgggactgcagtattaggggaacgagctacatggagagttaattatctctcccctcctacgggagtctcatgtgagctgtagaaagcggttggcacctctcgttacctcgcctgtacatgatcc

SEQ ID NO: 57 - DNA, 193 bp, Cricket paralysis virus
aaaagcaaaaatgtgatcttgcttgtaaatacaattttgagaggttaataaattacaagtagtgctatttttgtatttaggttagctatttagctttacgttccaggatgcctagtggcagccccacaatatccaggaagccctctctgcggtttttcagattaggtagtcgaaaaacctaagaaatttacct

SEQ ID NO: 58 - DNA, 752 bp, Unknown
  source: /note=“Description of Unknown Salivirus A sequence”
tttcctcctttcgaccgccttacggcaggcgggtccgcggacggcttcggcctacccgcgacaagaatgccgtcatctgtccttatcacccatattctttcccttcccccgcaaccatcacgcttactcgcgcacgtgttgagtggcacgtgcgttgtccaaacagttacactcacacccttggggcgggtttgtcccgccctcgggttcctcgcggaaccctccctcttctctctccctttctatccgccttcactttccataactacagtgctttggtaggtaagcatcctgaccccccgcggaagctgccaacgtggcaactgtggggatccaggcaggttatcaaaggcacccggtctttccgccttcaggagtatccctgccggtgaattccgacagggctctgcttggtgccaacctcccccaaatgcgcgctgcgggagtgctcttccccaactcatcttagtaacctctcatgtgtgtgcttggtcagcatatctgaggcgacgttccgctgtcccagaccagtccagcaatggacgggccagtgtgcgtagtcgctttccggtttcccggcgcatgtttggcgaaacgctgaggtaaggttggtgtgcccaatgcccgtaatttggtgacacctcaagaccacccaggaatgccagggaggtaccccacttcggtgggatctgaccctgggctaattgtctacggtggttcttcttgcttccacttctcttttttctggcatg

SEQ ID NO: 59 - DNA, 708 bp, Unknown
  source: /note=“Description of Unknown Salivirus A sequence”
tatggcaggcgggcttgtggacggcttcggcccacccacagcaagaatgccatcatctgtcctcacccccatgtttcccctttctttccctgcaaccgttacgcttactcgcaggtgcatttgagtggtgcacgtgttgaataaacagctacactcacatgggggcgggttttcccgccctgcggcctctcgcgaggcccacccctccccttcctcccataactacagtgctttggtaggtaagcatcctgatcccccgcggaagctgctcacgtggcaactgtggggacccagacaggttatcaaaggcacccggtctttccgccttcaggagtatccctgctagtgaattctagtagggctctgcttggtgccaacctcccccaaatgcgcgctgcgggagtgctcttccccaactcaccctagtatcctctcatgtgtgtgcttggtcagcatatctgagacgatgttccgctgtcccagaccagtccagtaatggacgggccagtgtgcgtagtcgtcttccggcttttccggcgcatgtttggtgaaccggtggggtaaggttggtgtgcccaacgcccgtactttggtgatacctcaagaccacccaggaatgccagggaggtaccccgcttcacagcgggatctgaccctgggctaattgtctacggtggttcttcttgcttccacttctttctactgttc

SEQ ID NO: 60 - DNA, 718 bp, Unknown
  source: /note=“Description of Unknown Salivirus A sequence”
tttcgaccgccttatggcaggcgggcttgtggacggcttcggcccacccacagcaagaatgccatcatctgtcctcacccccatttctcccctccttcccctgcaaccattacgcttactcgcatgtgcattgagtggtgcacgtgttgaacaaacagctacactcacgtgggggcgggttttcccgcccttcggcctctcgcgaggcccacccttccccttcctcccataactacagtgctttggtaggtaagcatcctgatcccccgcggaagctgctcgcgtggcaactgtggggacccagacaggttatcaaaggcacccggtctttccgcctccaggagtatccctgctagtgaattctagtggggctctgcttggtgccaacctcccccaaatgcgcgctgcgggagtgctcttccccaactcaccctagtatcctctcatgtgtgtgcttggtcagcatatctgagacgatgttccgctgtcccagaccagtccagcaatggacgggccagtgtgcgtagtcgtcttccggcttgtccggcgcatgtttggtgaaccggtggggtaaggttggtgtgcccaacgcccgtactttggtgacaactcaagaccacccaggaatgccagggaggtaccccgcctcacggcgggatctgaccctgggctaattgtctacggtggttcttcttgcttccatttctttcttctgttc

SEQ ID NO: 61 - DNA, 699 bp, Unknown
  source: /note=“Description of Unknown Salivirus A sequence”
tatggcaggcgggcttgtggacggtttcggcccacccacagcaagaatgccatcatctgtcctcacccccaattttccctttcttcccctgcaatcatcacgcttactcgcatgtgcattgagtggtgcatgtgttgaacaaacagctacactcacatgggggcgggttttcccgccctacggcctctcgcgaggcccacccttcccctccccttataactacagtgctttggcaggtaagcatcctgatcccccgcggaagctgctcacgtggcaactgtggggacccagacaggttatcaaaggcacccggtctttccgccttcaggagcatccccactagtgaattctagtggggctctgcttggtgccaacctcccccaaatgcgcgctgcgggagtgctcttccccaacccatcctagtatcctctcatgtgtgtgcttggtcagcatatctgagacgacgttccgctgtcccagaccagtccagtaatggacgggccagtgtgcgtagtcgtcttccggcttgtccggcgcatgtttggtgaaccggtggggtaaggttggtgtgcccaacgcccgtactttggtgacacctcaagaccacccaggaatgccagggaggtaccccgcctcacggcgggatctgaccctgggctaattgtctacggtggttcttcttgcttccacttctttctt

SEQ ID NO: 62 - DNA, 624 bp, Unknown
  source: /note=“Description of Unknown Salivirus A sequence”
ttctcctgcaaccattacgcttaatcgcatgtgcattgagtggtgcatgtgttgaacaaacagctacaatcacatgggggcgggttttcccgccccacggcttctcgcgaggcccatccctcccttttctcccataactacagtgctttggtaggtaagcatcccgatctcccgcggaagctgctcacgtggcaactgtggggacccagacaggttatcaaaggcacccggtctttccgccttcaggagtatccctgctagcgaattctagtagggctctgcttggtgccaacctctcccaaatgcgcgctgcgggagtgctcttccccaaatcaccccagtatcctctcatgtgtgtgcctggtcagcatatctgagacgatgttccgctgtcccagaccagtccagtaatggacgggccagtgtgcgtagtcgtcctccggcttgtccggcgcatgtttggtgaaccggtggggtaaggttggtgtgcccaacgcccgtaatcaggggatacctcaaggcacccaggaatgccagggaggtatcccgcctcacagcgggatctgaccctggggtaaatgtctgcggggggtcctcttggcccaattctcagtaattttcagg

SEQ ID NO: 63 - DNA, 650 bp, Unknown
  source: /note=“Description of Unknown Salivirus A sequence”
tctgtcctcaccccatcttcccttctttcctgcaccgttacgcttactcgcatgtgcattgagtggtgcacgtgcttgaacaaacagctacactcacatgggggcgggttttcccgccctgcggcctctcgcgaggcccacccctccccttcctcccataactacagtgctttggtaggtaagcatcctgatcccccgcggaagctgctcacgtggcaactgtggggacccagacaggttatcaaaggcacccggtctttccgccttcaggagtatccctgctagtgaattctagtagggctctgcttggtgccaacctcccccaaatgcgcgctgcgggagtgctcttccccaactcaccctagtatcctctcatgtgtgtgcttggtcagcatatctgagacgatgttccgctgtcccagaccagtccagtaatggacgggccagtgtgcgtagtcgtcttccggcttgtccggcgcatgtttggtgaaccggtggggtaaggttggtgtgcccaacgcccgtactttggtgatacctcaagaccacccaggaatgccagggaggtaccccgcttcacagcgggatctgaccctgggctaattgtctacggtggttcttcttgcttccacttctttctactgttcatg

SEQ ID NO: 64 - DNA, 730 bp, Unknown; identical to SEQ ID NO: 17
  source: /note=“Description of Unknown Salivirus FHB sequence”

SEQ ID NO: 65 - DNA, 741 bp, Unknown
  source: /note=“Description of Unknown CVB3 sequence”
ttaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaa

SEQ ID NO: 66 - DNA, 741 bp, Unknown
  source: /note=“Description of Unknown CVB1 sequence”
ttaaaacagcctgtgggttgttcccacccacaggcccattgggcgctagcactctggtatcacggtacctttgtgcgcctgttttacatcccctccccaaattgtaatttagaagtttcacacaccgatcattagcaagcgtggcacaccagccatgttttgatcaagcacttctgttaccccggactgagtatcaatagaccgctaacgcggttgaaggagaaaacgttcgttacccggccaactacttcgaaaaacctagtaacaccatggaagttgcggagtgtttcgctcagcactaccccagtgtagatcaggtcgatgagtcaccgcgttccccacgggcgaccgtggcggtggctgcgttggcggcctgcctacggggaaacccgtaggacgctctaatacagacatggtgcgaagagtctattgagctagttggtaatcctccggcccctgaatgcggctaatcctaactgcggagcacataccctcaaaccagggggcagtgtgtcgtaacgggcaactctgcagcggaaccgactactttgggtgtccgtgtttcattttattcctatactggctgcttatggtgacaattgacaggttgttaccatatagttattggattggccatccggtgactaacagagcaattatatatctctttgttgggtttataccacttagcttgaaagaggttaaaacactacatctcatcattaaactaaatacaacaaa

SEQ ID NO: 67 - DNA, 742 bp, Unknown
  source: /note=“Description of Unknown Echovirus 7 sequence”
ttaaaacagcctgtgggttgttcccacccacagggcccattgggcgtcagcaccctggtatcacggtacctttgtgcgcctgttttatatcccttcccccaattgtaacttagaagaaacacacaccgatcaacagcaagcgtggcacaccagccatgttttggtcaagcacttctgttaccccggactgagtatcaatagactgctcacgcggttgaaggagaaagcgtccgttatccggccagctacttcgagaaacctagtaacaccatggaagttgcggagtgtttcgctcagcactaccccagtgtagatcaggtcgatgagtcaccgctttccccacgggcgaccgtggcggtggctgcgttggcggcctgcctatgggggaacccataggacgctctaatacagacatggtgcgaagagtctattgagctagctggtattcctccggcccctgaatgcggctaatcctaactgtggagcacatgcccctaatccaaggggtagtgtgtcgtaatgagcaattccgcagcggaaccgactactttgggtgtccgtgtttcctcttattcttgtactggctgcttatggtgacaattgagagattgttaccatatagctattggattggccatccggtgactaatagagctattgtgtatctctttgttggatttgtaccacttaatttgaaagaaatcaggacactacgctacattttactattgaacaccgcaaa

SEQ ID NO: 68 - DNA, 743 bp, Unknown
  source: /note=“Description of Unknown CVB5 sequence”
ttaaaacagcctgtgggttgtacccacccacagggcccactgggcgctagcactctggtatcacggtacctttgtgcgcctgttttatgcccccttcccccaattgaaacttagaagttacacacaccgatcaacagcgggcgtggcataccagccgcgtcttgatcaagcactcctgtttccccggaccgagtatcaatagactgctcacgcggttgaaggagaaaacgttcgttacccggctaactacttcgagaaacctagtagcatcatgaaagttgcgaagcgtttcgctcagcacatccccagtgtagatcaggtcgatgagtcaccgcattccccacgggcgaccgtggcggtggctgcgttggcggcctgcctacggggcaacccgtaggacgcttcaatacagacatggtgcgaagagtcgattgagctagttagtagtcctccggcccctgaatccggctaatcctaactgcggagcacataccctcaacccagggggcattgtgtcgtaacgggtaactctgcagcggaaccgactactttgggtgtccgtgtttccttttattcttataatggctgcttatggtgacaattgaaagattgttaccatatagctattggattggccatccggtgtctaacagagctattatatacctctttgttggatttgtaccacttgatctaaaggaagtcaagacactacaattcatcatacaattgaacacagcaaa

SEQ ID NO: 69 - DNA, 743 bp, Unknown
  source: /note=“Description of Unknown Enterovirus A71 sequence”
ttaaaacagcctgtgggttgcacccactcacagggcccactgggcgcaagcactctggcacttcggtacctttgtgcgcctgttttatatcccctcccccaatgaaatttagaagcagcaaaccccgatcaatagcaggcataacgctccagttatgtcttgatcaagcacttctgtttccccggactgagtatcaatagactgctcacgcggttgaaggagaaaacgttcgttatccggctaactacttcggaaagcctagtaacaccatggaagttgcggagagtttcgttcagcacttccccagtgtagatcaggtcgatgagtcaccgcattccccacgggcgaccgtggcggtggctgcgttggcggcctgcccatggggtaacccatgggacgctctaatacggacatggtgtgaagagtctactgagctagttagtagtcctccggcccctgaatgcggctaatcccaactgcggagcacacgcccacaagccagtgggtagtgtgtcgtaacgggcaactctgcagcggaaccgactactttgggtgtccgtgtttccttttattcttatgttggctgcttatggtgacaattaaagagttgttaccatatagctattggattggccatccggtgtgcaacagagcgatcgtttacctatttattggttttgtaccattgacactgaagtctgtgatcacccttaattttatcttaaccctcaacacagccaaac

SEQ ID NO: 70 - DNA, 742 bp, Unknown
  source: /note=“Description of Unknown CVA3 sequence”
ttaaaacagcctgtgggttgtacccacccacagggcccactgggcgctagcacactggtattacggtacctttgtgcgcctgttttataccccccccaacctcgaaacttagaagtaaagcaaacccgatcaatagcaggtgcggcgcaccagtcgcatcttgatcaagcacttctgtaaccccggaccgagtatcaatagactgctcacgcggttgaaggagaaaacgttcgttacccggctaactacttcgagaaacccagtagcatcatgaaagttgcagagtgtttcgctcagcactacccccgtgtagatcaggccgatgagtcaccgcacttccccacgggcgaccgtggcggtggctgcgttggcggcctgcctatggggcaacccataggacgctctaatacggacatggtgcgaagagtctattgagctagttagtagtcctccggcccctgaatgcggctaatcctaactgcggagcacatacccttaatccaaagggcagtgtgtcgtaacgggtaactctgcagcggaaccgactactttgggtgtccgtgtttccttttaatttttactggctgcttatggtgacaattgaggaattgttgccatatagctattggattggccatccggtgactaacagagctattgtgttccaatttgttggatttaccccgctcacactcacagtcgtaagaacccttcattacgtgttatttctcaactcaagaaa

SEQ ID NO: 71 - DNA, 745 bp, Unknown
  source: /note=“Description of Unknown CVA12 sequence”
ttaaaacagcctgtgggttgtacccacccacagggcccactgggcgctagcactctggtactacggtacctttgtgtgcctgttttaagcccctaccccccactcgtaacttagaaggcttctcacactcgatcaatagtaggtgtggcacgccagtcacaccgtgatcaagcacttctgttaccccggtctgagtaccaataagctgctaacgcggctgaaggggaaaacgatcgttatccggctaactacttcgagaaacccagtaccaccatgaacgttgcagggtgtttcgctcggcacaaccccagtgtagatcaggtcgatgagtcaccgtattccccacgggcgaccgtggcggtggctgcgttggcggcctgcccatggggtgacccatgggacgctctaatactgacatggtgcgaagagtctattgagctagttagtagtcctccggcccctgaatgcggctaatcctaactgcggagcacatacccttaatccaaagggcagtgtgtcgtaacgggcaactctgcagcggaaccgactactttgggtgtccgtgtttccttttattcttacattggctgcttatggtgacaattgaaaagttgttaccatatagctattggattggccatccggtgacaaatagagctattgtatatctttttgttggttacgtaccccttaattacaaagtggtttcaactttgaaatacatcctaacactaaattgtagaaa

SEQ ID NO: 72 - DNA, 742 bp, Unknown
  source: /note=“Description of Unknown Echovirus E24 sequence”
ttaaaacagcctgtgggttgcacccacccacagggcccacagggcgctagcactctggtatcacggtacctttgtgcgcctgttttattaccccttccccaattgaaaattagaagcaatgcacaccgatcaacagcaggcgtggcgcaccagtcacgtctcgatcaagcacttctgtttccccggaccgagtatcaatagactgctcacgcggttgaaggagaaagtgttcgttatccggctaaccacttcgagaaacccagtaacaccatgaaagttgcagggtgtttcgctcagcacttccccagtgtagatcaggtcgatgagtcaccgcgttccccacgggcgaccgtggcggtggctgcgttggcggcctgcctatgggttaacccataggacgctctaatacagacatggtgcgaagagtttattgagctggttagtatccctccggcccctgaatgcggctaatcctaactgcggagcacgtgcctccaatccagggggttgcatgtcgtaacgggtaactctgcagcggaaccgactactttgggtgtccgtgtttccttttattcttatactggctgcttatggtgacaatcgaggaattgttaccatatagctattggattggccatccggtgtctaacagagcgattatatacctctttgttggatttatgcagctcaataccaccaactttaacacattgaaatatatcttaaagttaaacacagcaaa

SEQ ID NO: 73 - DNA, 240 bp, Anabaena sp.
gaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgt

SEQ ID NO: 74 - DNA, 239 bp, Anabaena sp.
aagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgt

SEQ ID NO: 75 - DNA, 238 bp, Anabaena sp.
agaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgt

SEQ ID NO: 76 - DNA, 190 bp, Anabaena sp.
gttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgt

SEQ ID NO: 77 - DNA, 189 bp, Anabaena sp.
ttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgt

SEQ ID NO: 78 - DNA, 188 bp, Anabaena sp.
tatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgt

SEQ ID NO: 79 - DNA, 187 bp, Anabaena sp.
atagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgt

SEQ ID NO: 80 - DNA, 186 bp, Anabaena sp.
tagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgt

SEQ ID NO: 81 - DNA, 139 bp, Anabaena sp.
acaatagatgacttacaactaatcggaaggtgcagagactcgacgggagctaccctaacgtcaagacgagggtaaagagagagtccaattctcaaagccaataggcagtagcgaaagctgcaagagaatgaaaatccgt

SEQ ID NO: 82 - DNA, 138 bp, Anabaena sp.
caatagatgacttacaactaatcggaaggtgcagagactcgacgggagctaccctaacgtcaagacgagggtaaagagagagtccaattctcaaagccaataggcagtagcgaaagctgcaagagaatgaaaatccgt

SEQ ID NO: 83 - DNA, 137 bp, Anabaena sp.
aatagatgacttacaactaatcggaaggtgcagagactcgacgggagctaccctaacgtcaagacgagggtaaagagagagtccaattctcaaagccaataggcagtagcgaaagctgcaagagaatgaaaatccgt

SEQ ID NO: 84 - DNA, 136 bp, Anabaena sp.
atagatgacttacaactaatcggaaggtgcagagactcgacgggagctaccctaacgtcaagacgagggtaaagagagagtccaattctcaaagccaataggcagtagcgaaagctgcaagagaatgaaaatccgt

SEQ ID NO: 85 - DNA, 135 bp, Anabaena sp.
tagatgacttacaactaatcggaaggtgcagagactcgacgggagctaccctaacgtcaagacgagggtaaagagagagtccaattctcaaagccaataggcagtagcgaaagctgcaagagaatgaaaatccgt

SEQ ID NO: 86 - DNA, 134 bp, Anabaena sp.
agatgacttacaactaatcggaaggtgcagagactcgacgggagctaccctaacgtcaagacgagggtaaagagagagtccaattctcaaagccaataggcagtagcgaaagctgcaagagaatgaaaatccgt

SEQ ID NO: 87 - DNA, 133 bp, Anabaena sp.
gatgacttacaactaatcggaaggtgcagagactcgacgggagctaccctaacgtcaagacgagggtaaagagagagtccaattctcaaagccaataggcagtagcgaaagctgcaagagaatgaaaatccgt

SEQ ID NO: 88 - DNA, 132 bp, Anabaena sp.
atgacttacaactaatcggaaggtgcagagactcgacgggagctaccctaacgtcaagacgagggtaaagagagagtccaattctcaaagccaataggcagtagcgaaagctgcaagagaatgaaaatccgt

SEQ ID NO: 89 - DNA, 131 bp, Anabaena sp.
tgacttacaactaatcggaaggtgcagagactcgacgggagctaccctaacgtcaagacgagggtaaagagagagtccaattctcaaagccaataggcagtagcgaaagctgcaagagaatgaaaatccgt

SEQ ID NO: 90 - DNA, 78 bp, Anabaena sp.
caagacgagggtaaagagagagtccaattctcaaagccaataggcagtagcgaaagctgcaagagaatgaaaatccgt

SEQ ID NO: 91 - DNA, 77 bp, Anabaena sp.
aagacgagggtaaagagagagtccaattctcaaagccaataggcagtagcgaaagctgcaagagaatgaaaatccgt

SEQ ID NO: 92 - DNA, 76 bp, Anabaena sp.
agacgagggtaaagagagagtccaattctcaaagccaataggcagtagcgaaagctgcaagagaatgaaaatccgt

SEQ ID NO: 93 - DNA, 75 bp, Anabaena sp.
gacgagggtaaagagagagtccaattctcaaagccaataggcagtagcgaaagctgcaagagaatgaaaatccgt

SEQ ID NO: 94 - DNA, 74 bp, Anabaena sp.
acgagggtaaagagagagtccaattctcaaagccaataggcagtagcgaaagctgcaagagaatgaaaatccgt

SEQ ID NO: 95 - DNA, 40 bp, Anabaena sp.
aataggcagtagcgaaagctgcaagagaatgaaaatccgt

SEQ ID NO: 96 - DNA, 39 bp, Anabaena sp.
ataggcagtagcgaaagctgcaagagaatgaaaatccgt

SEQ ID NO: 97 - DNA, 38 bp, Anabaena sp.
taggcagtagcgaaagctgcaagagaatgaaaatccgt

SEQ ID NO: 98 - DNA, 37 bp, Anabaena sp.
aggcagtagcgaaagctgcaagagaatgaaaatccgt

SEQ ID NO: 99 - DNA, 36 bp, Anabaena sp.
ggcagtagcgaaagctgcaagagaatgaaaatccgt

SEQ ID NO: 100 - DNA, 27 bp, Anabaena sp.
gaaagctgcaagagaatgaaaatccgt

SEQ ID NO: 101 - DNA, 26 bp, Anabaena sp.
aaagctgcaagagaatgaaaatccgt

SEQ ID NO: 102 - DNA, 25 bp, Anabaena sp.
aagctgcaagagaatgaaaatccgt

SEQ ID NO: 103 - DNA, 24 bp, Anabaena sp.
agctgcaagagaatgaaaatccgt

SEQ ID NO: 104 - DNA, 23 bp, Anabaena sp.
gctgcaagagaatgaaaatccgt

SEQ ID NO: 105 - DNA, 22 bp, Anabaena sp.
ctgcaagagaatgaaaatccgt

SEQ ID NO: 106 - DNA, 18 bp, Anabaena sp.
aagagaatgaaaatccgt

SEQ ID NO: 107 - DNA, 17 bp, Anabaena sp.
agagaatgaaaatccgt

SEQ ID NO: 108 - DNA, 16 bp, Anabaena sp.
gagaatgaaaatccgt

SEQ ID NO: 109 - DNA, 35 bp, Anabaena sp.
gcagtagcgaaagctgcaagagaatgaaaatccgt

SEQ ID NO: 110 - DNA, 33 bp, Anabaena sp.
agtagcgaaagctgcaagagaatgaaaatccgt

SEQ ID NO: 111 - DNA, 32 bp, Anabaena sp.
gtagcgaaagctgcaagagaatgaaaatccgt

SEQ ID NO: 112 - DNA, 26 bp, Anabaena sp.
acggacttaaataattgagccttaaa

SEQ ID NO: 113 - DNA, 27 bp, Anabaena sp.
acggacttaaataattgagccttaaag

SEQ ID NO: 114 - DNA, 28 bp, Anabaena sp.
acggacttaaataattgagccttaaaga

SEQ ID NO: 115 - DNA, 76 bp, Anabaena sp.
acggacttaaataattgagccttaaagaagaaattctttaagtggatgctctcaaactcagggaaacctaaatcta

SEQ ID NO: 116 - DNA, 77 bp, Anabaena sp.
acggacttaaataattgagccttaaagaagaaattctttaagtggatgctctcaaactcagggaaacctaaatctag

SEQ ID NO: 117 - DNA, 78 bp, Anabaena sp.
acggacttaaataattgagccttaaagaagaaattctttaagtggatgctctcaaactcagggaaacctaaatctagt

SEQ ID NO: 118 - DNA, 79 bp, Anabaena sp.
acggacttaaataattgagccttaaagaagaaattctttaagtggatgctctcaaactcagggaaacctaaatctagtt

SEQ ID NO: 119 - DNA, 80 bp, Anabaena sp.
acggacttaaataattgagccttaaagaagaaattctttaagtggatgctctcaaactcagggaaacctaaatctagtta

SEQ ID NO: 120 - DNA, 127 bp, Anabaena sp.
acggacttaaataattgagccttaaagaagaaattctttaagtggatgctctcaaactcagggaaacctaaatctagttatagacaaggcaatcctgagccaagccgaagtagtaattagtaagtta

SEQ ID NO: 121 - DNA, 128 bp, Anabaena sp.
acggacttaaataattgagccttaaagaagaaattctttaagtggatgctctcaaactcagggaaacctaaatctagttatagacaaggcaatcctgagccaagccgaagtagtaattagtaagttaa

SEQ ID NO: 122 - DNA, 129 bp, Anabaena sp.
acggacttaaataattgagccttaaagaagaaattctttaagtggatgctctcaaactcagggaaacctaaatctagttatagacaaggcaatcctgagccaagccgaagtagtaattagtaagttaac

SEQ ID NO: 123 - DNA, 130 bp, Anabaena sp.
acggacttaaataattgagccttaaagaagaaattctttaagtggatgctctcaaactcagggaaacctaaatctagttatagacaaggcaatcctgagccaagccgaagtagtaattagtaagttaaca

SEQ ID NO: 124 - DNA, 131 bp, Anabaena sp.
acggacttaaataattgagccttaaagaagaaattctttaagtggatgctctcaaactcagggaaacctaaatctagttatagacaaggcaatcctgagccaagccgaagtagtaattagtaagttaacaa

SEQ ID NO: 125 - DNA, 132 bp, Anabaena sp.
acggacttaaataattgagccttaaagaagaaattctttaagtggatgctctcaaactcagggaaacctaaatctagttatagacaaggcaatcctgagccaagccgaagtagtaattagtaagttaacaat

SEQ ID NO: 126 - DNA, 133 bp, Anabaena sp.
acggacttaaataattgagccttaaagaagaaattctttaagtggatgctctcaaactcagggaaacctaaatctagttatagacaaggcaatcctgagccaagccgaagtagtaattagtaagttaacaata

SEQ ID NO: 127 - DNA, 134 bp, Anabaena sp.
acggacttaaataattgagccttaaagaagaaattctttaagtggatgctctcaaactcagggaaacctaaatctagttatagacaaggcaatcctgagccaagccgaagtagtaattagtaagttaacaatag

SEQ ID NO: 128 - DNA, 135 bp, Anabaena sp.
acggacttaaataattgagccttaaagaagaaattctttaagtggatgctctcaaactcagggaaacctaaatctagttatagacaaggcaatcctgagccaagccgaagtagtaattagtaagttaacaataga

SEQ ID NO: 129 - DNA, 188 bp, Anabaena sp.
acggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgt

SEQ ID NO: 130 - DNA, 189 bp, Anabaena sp.
acggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtc

SEQ ID NO: 131 - DNA, 190 bp, Anabaena sp.
acggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtca

SEQ ID NO: 132 - DNA, 191 bp, Anabaena sp.
acggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaa

SEQ ID NO: 133 - DNA, 192 bp, Anabaena sp.
acggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaag

SEQ ID NO: 134 - DNA, 226 bp, Anabaena sp.
acggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagcc

SEQ ID NO: 135 - DNA, 227 bp, Anabaena sp.
acggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagcca

SEQ ID NO: 136 - DNA, 228 bp, Anabaena sp.
acggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaa

SEQ ID NO: 137 - DNA, 229 bp, Anabaena sp.
acggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaat

SEQ ID NO: 138 - DNA, 230 bp, Anabaena sp.
acggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaata

SEQ ID NO: 139 - DNA, 239 bp, Anabaena sp.
acggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaataggcagtagc

SEQ ID NO: 140 - DNA, 240 bp, Anabaena sp.
acggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaataggcagtagcg

SEQ ID NO: 141 - DNA, 241 bp, Anabaena sp.
acggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaataggcagtagcga

SEQ ID NO: 142 - DNA, 242 bp, Anabaena sp.
acggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaataggcagtagcgaa

SEQ ID NO: 143 - DNA, 243 bp, Anabaena sp.
acggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaataggcagtagcgaaa

SEQ ID NO: 144 - DNA, 244 bp, Anabaena sp.
acggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaataggcagtagcgaaag

SEQ ID NO: 145 - DNA, 248 bp, Anabaena sp.
acggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaataggcagtagcgaaagctgc

SEQ ID NO: 146 - DNA, 249 bp, Anabaena sp.
acggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaataggcagtagcgaaagctgca

SEQ ID NO: 147 - DNA, 250 bp, Anabaena sp.
acggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaataggcagtagcgaaagctgcaa

SEQ ID NO: 148 - DNA, 231 bp, Anabaena sp.
acggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaatag

SEQ ID NO: 149 - DNA, 233 bp, Anabaena sp.
acggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaataggc

SEQ ID NO: 150 - DNA, 234 bp, Anabaena sp.
acggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaataggca

SEQ ID NO: 151 - DNA, 104 bp, Azoarcus sp.
tgcgccgatgaaggtgtagagactagacggcacccacctaaggcaaacgctatggtgaaggcatagtccagggagtggcgaaagtcacacaaaccggaatccgt

SEQ ID NO: 152 - DNA, 141 bp, Azoarcus sp.
ccgggcgtatggcaacgccgagccaagcttcggcgcctgcgccgatgaaggtgtagagactagacggcacccacctaaggcaaacgctatggtgaaggcatagtccagggagtggcgaaagtcacacaaaccggaatccgt

SEQ ID NO: 153 - DNA, 78 bp, Azoarcus sp.
acggcacccacctaaggcaaacgctatggtgaaggcatagtccagggagtggcgaaagtcacacaaaccggaatccgt

SEQ ID NO: 154 - DNA, 58 bp, Azoarcus sp.
acgctatggtgaaggcatagtccagggagtggcgaaagtcacacaaaccggaatccgt

SEQ ID NO: 155 - DNA, 75 bp, Thiomargarita sp.
attaaagttatagaattatcagagaatgatatagtccaagccttatggtaacatgagggcacttgaccctggtag

SEQ ID NO: 156 - DNA, 195 bp, Staphylococcus virus Twort
aagatgtaggcaatcctgagctaagctcttagtaataagagaaagtgcaacgactattccgataggaagtagggtcaagtgactcgaaatggggattacccttctagggtagtgatatagtctgaacatatatggaaacatatagaaggataggagtaacgaacctattcgtaacataattgaacttttagttat

SEQ ID NO: 157 - DNA, 163 bp, Staphylococcus virus Twort
taataagagaaagtgcaacgactattccgataggaagtagggtcaagtgactcgaaatggggattacccttctagggtagtgatatagtctgaacatatatggaaacatatagaaggataggagtaacgaacctattcgtaacataattgaacttttagttat

SEQ ID NO: 158 - DNA, 133 bp, Staphylococcus virus Twort
taggaagtagggtcaagtgactcgaaatggggattacccttctagggtagtgatatagtctgaacatatatggaaacatatagaaggataggagtaacgaacctattcgtaacataattgaacttttagttat

SEQ ID NO: 159 - DNA, 92 bp, Staphylococcus virus Twort
ctagggtagtgatatagtctgaacatatatggaaacatatagaaggataggagtaacgaacctattcgtaacataattgaacttttagttat

SEQ ID NO: 160 - DNA, 92 bp, Unknown
  source: /note=“Description of Unknown LSUp1 sequence”
agttaataaagatgatgaaatagtctgaaccattttgagaaaagtggaaataaaagaaaatcttttatgataacataaattgaacaggctaa

SEQ ID NO: 161 - DNA, 62 bp, Unknown
  source: /note=“Description of Unknown Bacteriophage Phi I sequence”
caaagactgatgatatagtccgacactcctagtaataggagaatacagaaaggatgaaatcc

SEQ ID NO: 162 - DNA, 76 bp, Nostoc sp.
agtcgagggtaaagggagagtccaattctcaaagcctattggcagtagcgaaagctgcgggagaatgaaaatccgt

SEQ ID NO: 163 - DNA, 76 bp, Nostoc sp.
agccgagggtaaagggagagtccaattctcaaagccaataggcagtagcgaaagctgcgggagaatgaaaatccgt

SEQ ID NO: 164 - DNA, 89 bp, Nodularia sp.
agccgagggtaaagggagagtccaattctcaaagccgaaggttattaaaacctggcagcagtgaaagctgcgggagaatgaaaatccgt

SEQ ID NO: 165 - DNA, 79 bp, Pleurocapsa sp.
agctgagggtaaagagagagtccaattctcaaagccagcagatggcagtagcgaaagctgcgggagaatgaaaatccgt

SEQ ID NO: 166 - DNA, 76 bp, Planktothrix sp.
agccgagggtaaagagagagtccaattctcaaagccaattggtagtagcgaaagctacgggagaatgaaaatccgt

SEQ ID NO: 167 - DNA, 118 bp, Azoarcus sp.
gcggactcatatttcgatgtgccttgcgccgggaaaccacgcaagggatggtgtcaaattcggcgaaacctaagcgcccgcccgggcgtatggcaacgccgagccaagcttcggcgcc

SEQ ID NO: 168 - DNA, 81 bp, Azoarcus sp.
gcggactcatatttcgatgtgccttgcgccgggaaaccacgcaagggatggtgtcaaattcggcgaaacctaagcgcccgc

SEQ ID NO: 169 - DNA, 144 bp, Azoarcus sp.
gcggactcatatttcgatgtgccttgcgccgggaaaccacgcaagggatggtgtcaaattcggcgaaacctaagcgcccgcccgggcgtatggcaacgccgagccaagcttcggcgcctgcgccgatgaaggtgtagagactag

SEQ ID NO: 170 - DNA, 164 bp, Azoarcus sp.
gcggactcatatttcgatgtgccttgcgccgggaaaccacgcaagggatggtgtcaaattcggcgaaacctaagcgcccgcccgggcgtatggcaacgccgagccaagcttcggcgcctgcgccgatgaaggtgtagagactagacggcacccacctaaggcaa

SEQ ID NO: 171 - DNA, 179 bp, Thiomargarita sp.
aggattagatactacactaagtgtcccccagactggtgacagtctggtgtgcatccagctatatcggtgaaaccccattggggtaataccgagggaagctatattatatatatattaataaatagccccgtagagactatgtaggtaaggagatagaagatgataaaatcaaaatcatc

SEQ ID NO: 172 - DNA, 84 bp, Staphylococcus virus Twort
actactgaaagcataaataattgtgcctttatacagtaatgtatatcgaaaaatcctctaattcagggaacacctaaacaaact

SEQ ID NO: 173 - DNA, 116 bp, Staphylococcus virus Twort
actactgaaagcataaataattgtgcctttatacagtaatgtatatcgaaaaatcctctaattcagggaacacctaaacaaactaagatgtaggcaatcctgagctaagctcttag

SEQ ID NO: 174 - DNA, 146 bp, Staphylococcus virus Twort
actactgaaagcataaataattgtgcctttatacagtaatgtatatcgaaaaatcctctaattcagggaacacctaaacaaactaagatgtaggcaatcctgagctaagctcttagtaataagagaaagtgcaacgactattccga

SEQ ID NO: 175 - DNA, 187 bp, Staphylococcus virus Twort
actactgaaagcataaataattgtgcctttatacagtaatgtatatcgaaaaatcctctaattcagggaacacctaaacaaactaagatgtaggcaatcctgagctaagctcttagtaataagagaaagtgcaacgactattccgataggaagtagggtcaagtgactcgaaatggggattaccctt

SEQ ID NO: 176 - DNA, 216 bp, Unknown
  source: /note=“Description of Unknown LSUp1 sequence”
cgctagggatttataactgtgagtcctccaatattataaaatgttggtaatatattgggtaaatttcaaagacaacttttctccacgtcaggatatagtgtatttgaagcgaaacttattttagcagtgaaaaagcaaataaggacgttcaacgactaaaaggtgagtattgctaacaataatccttttttttaatgcccaacatctttattaact

SEQ ID NO: 177 - DNA, 559 bp, Unknown
  source: /note=“Description of Unknown Bacteriophage Phi I sequence”
gtgggtgcataaactatttcattgtgcacattaaatctggtgaactcggtgaaaccctaatggggcaataccgagccaagccatagggaggatatatgagaggcaagaagttaattcttgaggccactgagactggctgtatcatccctacgtcacacaaacttaatgccgatggttatttcagaaagaaaaccaatggcgtcttagagatgtatcacagaacggtgtggaaggagcataacggagacatacctgatggcttcgagatagaccataagtgtcgcaatagggcttgctgtaatatagagcatttacagatgcttgagggtacagcccacactgttaagaccaatcgtgaacgctacgcagacagaaaggaaacagctagggaatactggctggagactggatgtaccggcctagcactcggtgagaagtttggtgtgtcgttctcttctgcttgtaagtggattagagaatggaaggcgtagagactatccgaaaggagtagggccgagggtgagactccctcgtaacccgaagcgccagacagtcaact

SEQ ID NO: 178 - DNA, 218 bp, Nostoc sp.
acggacttaagtaattgagccttaaagaagaaattctttaagtggcagctctcaaactcagggaaacctaaatctgttcacagacaaggcaatcctgagccaagccgaaagagtcatgagtgctgagtagtgagtaaaataaaagctcacaactcagaggttgtaactctaagctagtcggaaggtgcagagactcgacgggagctaccctaacgtaa

SEQ ID NO: 179 - DNA, 165 bp, Nostoc sp.
acggacttaaactgaattgagccttagagaagaaattctttaagtgtcagctctcaaactcagggaaacctaaatctgttgacagacaaggcaatcctgagccaagccgagaactctaagttattcggaaggtgcagagactcgacgggagctaccctaacgtca

SEQ ID NO: 180 - DNA, 181 bp, Nodularia sp.
acggacttagaaaactgagccttgatcgagaaatctttcaagtggaagctctcaaattcagggaaacctaaatctgtttacagatatggcaatcctgagccaagccgaaacaagtcctgagtgttaaagctcataactcatcggaaggtgcagagactcgacgggagctaccctaacgtta

SEQ ID NO: 181 - DNA, 188 bp, Pleurocapsa sp.
acggacttaaaaaaattgagccttggcagagaaatctgtcatgcgaacgctctcaaattcagggaaacctaagtctggcaacagatatggcaatcctgagccaagccttaatcaaggaaaaaaacatttttaccttttaccttgaaaggaaggtgcagagactcaacgggagctaccctaacaggtca

SEQ ID NO: 182 - DNA, 200 bp, Planktothrix sp.
acggacttaaagataaattgagccttgaggcgagaaatctctcaagtgtaagctgtcaaattcagggaaacctaaatctgtaaattcagacaaggcaatcctgagccaagcctaggggtattagaaatgagggagtttccccaatctaagatcaatacctaggaaggtgcagagactcgacgggagctaccctaacgtta

SEQ ID NO: 183 - DNA, 166 bp, Artificial Sequence
  source: /note=“Description of Artificial Sequence Synthetic polynucleotide”
agtatataagaaacaaaccactagatgacttacaactaatcggaaggtgcagagactcgacgggagctaccctaacgtcaagacgagggtaaagagagagtccaattctcaaagccaataggcagtagcgaaagctgcaagagaatgaaaatccgtggctcgcagc

SEQ ID NO: 184 - DNA, 176 bp, Artificial Sequence
  source: /note=“Description of Artificial Sequence Synthetic polynucleotide”
ctgaaattatacttatactcaaacaaaccactagatgacttacaactaatcggaaggtgcagagactcgacgggagctaccctaacgtcaagacgagggtaaagagagagtccaattctcaaagccaataggcagtagcgaaagctgcaagagaatgaaaatccgtggctcgcagc

SEQ ID NO: 185 - DNA, 186 bp, Artificial Sequence
  source: /note=“Description of Artificial Sequence Synthetic polynucleotide”
ctgaaattatacttatactcagtatatgacaaacaaaccactagatgacttacaactaatcggaaggtgcagagactcgacgggagctaccctaacgtcaagacgagggtaaagagagagtccaattctcaaagccaataggcagtagcgaaagctgcaagagaatgaaaatccgtggctcgcagc

SEQ ID NO: 186 - DNA, 196 bp, Artificial Sequence
  source: /note=“Description of Artificial Sequence Synthetic polynucleotide”
catcaacaatatgaaattatacttatactcagtatatgacaaacaaaccactagatgacttacaactaatcggaaggtgcagagactcgacgggagctaccctaacgtcaagacgagggtaaagagagagtccaattctcaaagccaataggcagtagcgaaagctgcaagagaatgaaaatccgtggctcgcagc

SEQ ID NO: 187 - DNA, 206 bp, Artificial Sequence
  source: /note=“Description of Artificial Sequence Synthetic polynucleotide”
catcaacaatatgaaactatacttatactcagtatatgaagcattatcgcaaacaaaccactagatgacttacaactaatcggaaggtgcagagactcgacgggagctaccctaacgtcaagacgagggtaaagagagagtccaattctcaaagccaataggcagtagcgaaagctgcaagagaatgaaaatccgtggctcgcagc

SEQ ID NO: 188 - DNA, 166 bp, Artificial Sequence
  source: /note=“Description of Artificial Sequence Synthetic polynucleotide”
tagcgtcagcaaacaaacaaatagatgacttacaactaatcggaaggtgcagagactcgacgggagctaccctaacgtcaagacgagggtaaagagagagtccaattctcaaagccaataggcagtagcgaaagctgcaagagaatgaaaatccgtggctcgcagc

SEQ ID NO: 189 - DNA, 176 bp, Artificial Sequence
  source: /note=“Description of Artificial Sequence Synthetic polynucleotide”
atactcatactagcgtcagcaaacaaacaaatagatgacttacaactaatcggaaggtgcagagactcgacgggagctaccctaacgtcaagacgagggtaaagagagagtccaattctcaaagccaataggcagtagcgaaagctgcaagagaatgaaaatccgtggctcgcagc

SEQ ID NO: 190 - DNA, 186 bp, Artificial Sequence
  source: /note=“Description of Artificial Sequence Synthetic polynucleotide”
gtgtgaagctatactcatactagcgtcagcaaacaaacaaatagatgacttacaactaatcggaaggtgcagagactcgacgggagctaccctaacgtcaagacgagggtaaagagagagtccaattctcaaagccaataggcagtagcgaaagctgcaagagaatgaaaatccgtggctcgcagc

SEQ ID NO: 191 - DNA, 196 bp, Artificial Sequence
  source: /note=“Description of Artificial Sequence Synthetic polynucleotide”
cctcacctgagtgtgaagctatactcatactagcgtcagcaaacaaacaaatagatgacttacaactaatcggaaggtgcagagactcgacgggagctaccctaacgtcaagacgagggtaaagagagagtccaattctcaaagccaataggcagtagcgaaagctgcaagagaatgaaaatccgtggctcgcagc

SEQ ID NO: 192 - DNA, 206 bp, Artificial Sequence
  source: /note=“Description of Artificial Sequence Synthetic polynucleotide”
ccgaatgatgcctcacctgagtgtgaagctatactcatactagcgtcagcaaacaaacaaatagatgacttacaactaatcggaaggtgcagagactcgacgggagctaccctaacgtcaagacgagggtaaagagagagtccaattctcaaagccaataggcagtagcgaaagctgcaagagaatgaaaatccgtggctcgcagc

SEQ ID NO: 193 - DNA, 166 bp, Artificial Sequence
  source: /note=“Description of Artificial Sequence Synthetic polynucleotide”
cggtgcgagcaaacaaacaaatagatgacttacaactaatcggaaggtgcagagactcgacgggagctaccctaacgtcaagacgagggtaaagagagagtccaattctcaaagccaataggcagtagcgaaagctgcaagagaatgaaaatccgtggctcgcagc

SEQ ID NO: 194 - DNA, 176 bp, Artificial Sequence
  source: /note=“Description of Artificial Sequence Synthetic polynucleotide”
cgctccgacccagtgcgagcaaacaaacaaatagatgacttacaactaatcggaaggtgcagagactcgacgggagctaccctaacgtcaagacgagggtaaagagagagtccaattctcaaagccaataggcagtagcgaaagctgcaagagaatgaaaatccgtggctcgcagc

SEQ ID NO: 195 - DNA, 186 bp, Artificial Sequence
  source: /note=“Description of Artificial Sequence Synthetic polynucleotide”
ctgaaattatactaatactcagtatatgacaaacaaaccactagatgacttacaactaatcggaaggtgcagagactcgacgggagctaccctaacgtcaagacgagggtaaagagagagtccaattctcaaagccaataggcagtagcgaaagctgcaagagaatgaaaatccgtggctcgcagc

SEQ ID NO: 196 - DNA, 186 bp, Artificial Sequence
  source: /note=“Description of Artificial Sequence Synthetic polynucleotide”
ctgaaaatatactaatactcactatatgacaaacaaaccactagatgacttacaactaatcggaaggtgcagagactcgacgggagctaccctaacgtcaagacgagggtaaagagagagtccaattctcaaagccaataggcagtagcgaaagctgcaagagaatgaaaatccgtggctcgcagc

SEQ ID NO: 197 - DNA, 186 bp, Artificial Sequence
  source: /note=“Description of Artificial Sequence Synthetic polynucleotide”
ctgataatatagtaatactcactatatgacaaacaaaccactagatgacttacaactaatcggaaggtgcagagactcgacgggagctaccctaacgtcaagacgagggtaaagagagagtccaattctcaaagccaataggcagtagcgaaagctgcaagagaatgaaaatccgtggctcgcagc

SEQ ID NO: 198 - DNA, 186 bp, Artificial Sequence
  source: /note=“Description of Artificial Sequence Synthetic polynucleotide”
ctgataataaagtaatacacactataagacaaacaaaccactagatgacttacaactaatcggaaggtgcagagactcgacgggagctaccctaacgtcaagacgagggtaaagagagagtccaattctcaaagccaataggcagtagcgaaagctgcaagagaatgaaaatccgtggctcgcagc

SEQ ID NO: 199 - DNA, 186 bp, Artificial Sequence
  source: /note=“Description of Artificial Sequence Synthetic polynucleotide”
ctgaaattatacttatactctctaagttacaaacaaaccactagatgacttacaactaatcggaaggtgcagagactcgacgggagctaccctaacgtcaagacgagggtaaagagagagtccaattctcaaagccaataggcagtagcgaaagctgcaagagaatgaaaatccgtggctcgcagc

SEQ ID NO: 200 - DNA, 186 bp, Artificial Sequence
  source: /note=“Description of Artificial Sequence Synthetic polynucleotide”
ctgaaattatgtgtgttacatctaagttacaaacaaaccactagatgacttacaactaatcggaaggtgcagagactcgacgggagctaccctaacgtcaagacgagggtaaagagagagtccaattctcaaagccaataggcagtagcgaaagctgcaagagaatgaaaatccgtggctcgcagc

SEQ ID NO: 201 - DNA, 186 bp, Artificial Sequence
  source: /note=“Description of Artificial Sequence Synthetic polynucleotide”
gttgatcggtgtgtgttacatctaagttacaaacaaaccactagatgacttacaactaatcggaaggtgcagagactcgacgggagctaccctaacgtcaagacgagggtaaagagagagtccaattctcaaagccaataggcagtagcgaaagctgcaagagaatgaaaatccgtggctcgcagc

SEQ ID NO: 202 - DNA, 186 bp, Artificial Sequence
  source: /note=“Description of Artificial Sequence Synthetic polynucleotide”
ctgaaattatacttatactcagtatatgacaaacaaaccactagatgacttacaactaatcggaaggtgcagagactcgacgggagctaccctaacgtcaagacgagggtaaagagagagtccaattctcaaagccaataggcagtagcgaaagctgcaagagaatgaaaatccgtgattaaacag

SEQ ID NO: 203 - DNA, 196 bp, Artificial Sequence
  source: /note=“Description of Artificial Sequence Synthetic polynucleotide”
ctgaaattatacttatactcagtatatgacaaacaaaccactagatgacttacaactaatcggaaggtgcagagactcgacgggagctaccctaacgtcaagacgagggtaaagagagagtccaattctcaaagccaataggcagtagcgaaagctgcaagagaatgaaaatccgtgattcacaatataaattacg

SEQ ID NO: 204 - DNA, 186 bp, Artificial Sequence
  source: /note=“Description of Artificial Sequence Synthetic polynucleotide”
ctgaaattatacttatactcagtatatgacaaacaaaccactagatgacttacaactaatcggaaggtgcagagactcgacgggagctaccctaacgtcaagacgagggtaaagagagagtccaattctcaaagccaataggcagtagcgaaagctgcaagagaatgaaaatccgtggatcatagc

SEQ ID NO: 205 - DNA, 196 bp, Artificial Sequence
  source: /note=“Description of Artificial Sequence Synthetic polynucleotide”
ctgaaattatacttatactcagtatatgacaaacaaaccactagatgacttacaactaatcggaaggtgcagagactcgacgggagctaccctaacgtcaagacgagggtaaagagagagtccaattctcaaagccaataggcagtagcgaaagctgcaagagaatgaaaatccgtggatcgcagcataatatccg

SEQ ID NO: 206 - DNA, 196 bp, Artificial Sequence
  source: /note=“Description of Artificial Sequence Synthetic polynucleotide”
ctgaaattatacttatactcagtatatgacaaacaaaccactagatgacttacaactaatcggaaggtgcagagactcgacgggagctaccctaacgtcaagacgagggtaaagagagagtccaattctcaaagccaataggcagtagcgaaagctgcaagagaatgaaaatccgtggctcgcagcgcgcctaccg

SEQ ID NO: 207 - DNA, 219 bp, Artificial Sequence
  source: /note=“Description of Artificial Sequence Synthetic polynucleotide”
ctgaaattatacttatactcagtatatgacaaacaaaccactagatgacttacaactaatcggaaggtgcagagactcgacgggagctaccctaacgtcaagacgagggtaaagagagagtccaattctcaaagccaataggcagtagcgaaagctgcaagagaatgaaaatccgtggctcgcagcgcgcctaccgaaagccggcgtcgacgttagcgc

SEQ ID NO: 208 - DNA, 219 bp, Artificial Sequence
  source: /note=“Description of Artificial Sequence Synthetic polynucleotide”
ctgaaattatacttatactcagtatatgacaaacaaaccactagatgacttacaactaatcggaaggtgcagagactcgacgggagctaccctaacgtcaagacgagggtaaagagagagtccaattctcaaagccaataggcagtagcgaaagctgcaagagaatgaaaatccgtggatcgcagcataatatccgaaacgaggatacaagtgacatgc

SEQ ID NO: 209 - DNA, 219 bp, Artificial Sequence
  source: /note=“Description of Artificial Sequence Synthetic polynucleotide”
ctgaaattatacttatactcagtatatgacaaacaaaccactagatgacttacaactaatcggaaggtgcagagactcgacgggagctaccctaacgtcaagacgagggtaaagagagagtccaattctcaaagccaataggcagtagcgaaagctgcaagagaatgaaaatccgtgattcacaatctaaattacgaaacgataaatgataactctaac

SEQ ID NO: 210 - DNA, 156 bp, Artificial Sequence
  source: /note=“Description of Artificial Sequence Synthetic polynucleotide”
aaacaaaccactagatgacttacaactaatcggaaggtgcagagactcgacgggagctaccctaacgtcaagacgagggtaaagagagagtccaattctcaaagccaataggcagtagcgaaagctgcaagagaatgaaaatccgtggctcgcagc

SEQ ID NO: 211 - DNA, 161 bp, Artificial Sequence
  source: /note=“Description of Artificial Sequence Synthetic polynucleotide”
cgggcaaacaaacaaatagatgacttacaactaatcggaaggtgcagagactcgacgggagctaccctaacgtcaagacgagggtaaagagagagtccaattctcaaagccaataggcagtagcgaaagctgcaagagaatgaaaatccgtggctcgcagc

SEQ ID NO: 212 - DNA, 186 bp, Artificial Sequence
  source: /note=“Description of Artificial Sequence Synthetic polynucleotide”
cgctccgacgagcttccggccagtgcgagcaaacaaacaaatagatgacttacaactaatcggaaggtgcagagactcgacgggagctaccctaacgtcaagacgagggtaaagagagagtccaattctcaaagccaataggcagtagcgaaagctgcaagagaatgaaaatccgtggctcgcagc

SEQ ID NO: 213 - DNA, 57 bp, Artificial Sequence
  source: /note=“Description of Artificial Sequence Synthetic oligonucleotide”
aaacaaaccacggcagtagcgaaagctgcaagagaatgaaaatccgtggctcgcagc

SEQ ID NO: 214 - DNA, 67 bp, Artificial Sequence
  source: /note=“Description of Artificial Sequence Synthetic oligonucleotide”
agtatataagaaacaaaccacggcagtagcgaaagctgcaagagaatgaaaatccgtggctcgcagc

SEQ ID NO: 215 - DNA, 77 bp, Artificial Sequence
  source: /note=“Description of Artificial Sequence Synthetic oligonucleotide”
ctgaaattatacttatactcaaacaaaccacggcagtagcgaaagctgcaagagaatgaaaatccgtggctcgcagc

SEQ ID NO: 216 - DNA, 87 bp, Artificial Sequence
  source: /note=“Description of Artificial Sequence Synthetic oligonucleotide”
ctgaaattatacttatactcagtatatgacaaacaaaccacggcagtagcgaaagctgcaagagaatgaaaatccgtggctcgcagc

SEQ ID NO: 217 - DNA, 67 bp, Artificial Sequence
  source: /note=“Description of Artificial Sequence Synthetic oligonucleotide”
tagcgtcagcaaacaaacaaaggcagtagcgaaagctgcaagagaatgaaaatccgtggctcgcagc

SEQ ID NO: 218 - DNA, 77 bp, Artificial Sequence
  source: /note=“Description of Artificial Sequence Synthetic oligonucleotide”
atactcatactagcgtcagcaaacaaacaaaggcagtagcgaaagctgcaagagaatgaaaatccgtggctcgcagc

SEQ ID NO: 219 - DNA, 87 bp, Artificial Sequence
  source: /note=“Description of Artificial Sequence Synthetic oligonucleotide”
gtgtgaagctatactcatactagcgtcagcaaacaaacaaaggcagtagcgaaagctgcaagagaatgaaaatccgtggctcgcagc

SEQ ID NO: 220 - DNA, 67 bp, Artificial Sequence
  source: /note=“Description of Artificial Sequence Synthetic oligonucleotide”
cggtgcgagcaaacaaacaaaggcagtagcgaaagctgcaagagaatgaaaatccgtggctcgcagc

SEQ ID NO: 221 - DNA, 77 bp, Artificial Sequence
  source: /note=“Description of Artificial Sequence Synthetic oligonucleotide”
cgctccgacccagtgcgagcaaacaaacaaaggcagtagcgaaagctgcaagagaatgaaaatccgtggctcgcagc

SEQ ID NO: 222 - DNA, 87 bp, Artificial Sequence
  source: /note=“Description of Artificial Sequence Synthetic oligonucleotide”
cgctccgacgagcttccggccagtgcgagcaaacaaacaaaggcagtagcgaaagctgcaagagaatgaaaatccgtggctcgcagc

SEQ ID NO: 223 - DNA, 98 bp, Artificial Sequence
  source: /note=“Description of Artificial Sequence Synthetic oligonucleotide”
aaacaaaccacaagacgagggtaaagagagagtccaattctcaaagccaataggcagtagcgaaagctgcaagagaatgaaaatccgtggctcgcagc

SEQ ID NO: 224 - DNA, 108 bp, Artificial Sequence
  source: /note=“Description of Artificial Sequence Synthetic polynucleotide”
agtatataagaaacaaaccacaagacgagggtaaagagagagtccaattctcaaagccaataggcagtagcgaaagctgcaagagaatgaaaatccgtggctcgcagc

SEQ ID NO: 225 - DNA, 118 bp, Artificial Sequence
  source: /note=“Description of Artificial Sequence Synthetic polynucleotide”
ctgaaattatacttatactcaaacaaaccacaagacgagggtaaagagagagtccaattctcaaagccaataggcagtagcgaaagctgcaagagaatgaaaatccgtggctcgcagc

SEQ ID NO: 226 - DNA, 128 bp, Artificial Sequence
  source: /note=“Description of Artificial Sequence Synthetic polynucleotide”
ctgaaattatacttatactcagtatatgacaaacaaaccacaagacgagggtaaagagagagtccaattctcaaagccaataggcagtagcgaaagctgcaagagaatgaaaatccgtggctcgcagc

SEQ ID NO: 227 - DNA, 108 bp, Artificial Sequence
  source: /note=“Description of Artificial Sequence Synthetic polynucleotide”
tagcgtcagcaaacaaacaaaaagacgagggtaaagagagagtccaattctcaaagccaataggcagtagcgaaagctgcaagagaatgaaaatccgtggctcgcagc

SEQ ID NO: 228 - DNA, 118 bp, Artificial Sequence
  source: /note=“Description of Artificial Sequence Synthetic polynucleotide”
atactcatactagcgtcagcaaacaaacaaaaagacgagggtaaagagagagtccaattctcaaagccaataggcagtagcgaaagctgcaagagaatgaaaatccgtggctcgcagc

SEQ ID NO: 229 - DNA, 128 bp, Artificial Sequence
  source: /note=“Description of Artificial Sequence Synthetic polynucleotide”
gtgtgaagctatactcatactagcgtcagcaaacaaacaaaaagacgagggtaaagagagagtccaattctcaaagccaataggcagtagcgaaagctgcaagagaatgaaaatccgtggctcgcagc

SEQ ID NO: 230 - DNA, 108 bp, Artificial Sequence
  source: /note=“Description of Artificial Sequence Synthetic polynucleotide”
cggtgcgagcaaacaaacaaaaagacgagggtaaagagagagtccaattctcaaagccaataggcagtagcgaaagctgcaagagaatgaaaatccgtggctcgcagc

SEQ ID NO: 231 - DNA, 118 bp, Artificial Sequence
  source: /note=“Description of Artificial Sequence Synthetic polynucleotide”
cgctccgacccagtgcgagcaaacaaacaaaaagacgagggtaaagagagagtccaattctcaaagccaataggcagtagcgaaagctgcaagagaatgaaaatccgtggctcgcagc

SEQ ID NO: 232 - DNA, 128 bp, Artificial Sequence
  source: /note=“Description of Artificial Sequence Synthetic polynucleotide”
cgctccgacgagcttccggccagtgcgagcaaacaaacaaaaagacgagggtaaagagagagtccaattctcaaagccaataggcagtagcgaaagctgcaagagaatgaaaatccgtggctcgcagc

SEQ ID NO: 233 - DNA, 176 bp, Artificial Sequence
  source: /note=“Description of Artificial Sequence Synthetic polynucleotide”
ctgaaattatacttatactcagtatatgacaaacaaaccactagatgacttacaactaatcggaaggtgcagagactcgacgggagctaccctaacgtcaagacgagggtaaagagagagtccaattctcaaagccaataggcagtagcgaaagctgcaagagaatgaaaatccgt

SEQ ID NO: 234 - DNA, 77 bp, Artificial Sequence
  source: /note=“Description of Artificial Sequence Synthetic oligonucleotide”
ctgaaattatacttatactcagtatatgacaaacaaaccacggcagtagcgaaagctgcaagagaatgaaaatccgt

SEQ ID NO: 235 - DNA, 87 bp, Artificial Sequence
  source: /note=“Description of Artificial Sequence Synthetic oligonucleotide”
ctgaaattatacttatactcagtatatgacaaacaaaccacggcagtagcgaaagctgcaagagaatgaaaatccgtgattaaacag

SEQ ID NO: 236 - DNA, 97 bp, Artificial Sequence
  source: /note=“Description of Artificial Sequence Synthetic oligonucleotide”
ctgaaattatacttatactcagtatatgacaaacaaaccacggcagtagcgaaagctgcaagagaatgaaaatccgtgattcacaatataaattacg

SEQ ID NO: 237 - DNA, 87 bp, Artificial Sequence
  source: /note=“Description of Artificial Sequence Synthetic oligonucleotide”
ctgaaattatacttatactcagtatatgacaaacaaaccacggcagtagcgaaagctgcaagagaatgaaaatccgtggatcatagc

SEQ ID NO: 238 - DNA, 97 bp, Artificial Sequence
  source: /note=“Description of Artificial Sequence Synthetic oligonucleotide”
ctgaaattatacttatactcagtatatgacaaacaaaccacggcagtagcgaaagctgcaagagaatgaaaatccgtggatcgcagcataatatccg

SEQ ID NO: 239 - DNA, 97 bp, Artificial Sequence
  source: /note=“Description of Artificial Sequence Synthetic oligonucleotide”
ctgaaattatacttatactcagtatatgacaaacaaaccacggcagtagcgaaagctgcaagagaatgaaaatccgtggctcgcagcgcgcctaccg

SEQ ID NO: 240 - DNA, 118 bp, Artificial Sequence
  source: /note=“Description of Artificial Sequence Synthetic polynucleotide”
ctgaaattatacttatactcagtatatgacaaacaaaccacaagacgagggtaaagagagagtccaattctcaaagccaataggcagtagcgaaagctgcaagagaatgaaaatccgt

SEQ ID NO: 241 - DNA, 128 bp, Artificial Sequence
  source: /note=“Description of Artificial Sequence Synthetic polynucleotide”
ctgaaattatacttatactcagtatatgacaaacaaaccacaagacgagggtaaagagagagtccaattctcaaagccaataggcagtagcgaaagctgcaagagaatgaaaatccgtgattaaacag

SEQ ID NO: 242 - DNA, 138 bp, Artificial Sequence
  source: /note=“Description of Artificial Sequence Synthetic polynucleotide”
ctgaaattatacttatactcagtatatgacaaacaaaccacaagacgagggtaaagagagagtccaattctcaaagccaataggcagtagcgaaagctgcaagagaatgaaaatccgtgattcacaatataaattacg

SEQ ID NO: 243 - DNA, 128 bp, Artificial Sequence
  source: /note=“Description of Artificial Sequence Synthetic polynucleotide”
ctgaaattatacttatactcagtatatgacaaacaaaccacaagacgagggtaaagagagagtccaattctcaaagccaataggcagtagcgaaagctgcaagagaatgaaaatccgtggatcatagc

SEQ ID NO: 244 - DNA, 138 bp, Artificial Sequence
  source: /note=“Description of Artificial Sequence Synthetic polynucleotide”
ctgaaattatacttatactcagtatatgacaaacaaaccacaagacgagggtaaagagagagtccaattctcaaagccaataggcagtagcgaaagctgcaagagaatgaaaatccgtggatcgcagcataatatccg

SEQ ID NO: 245 - DNA, 138 bp, Artificial Sequence
  source: /note=“Description of Artificial Sequence Synthetic polynucleotide”
ctgaaattatacttatactcagtatatgacaaacaaaccacaagacgagggtaaagagagagtccaattctcaaagccaataggcagtagcgaaagctgcaagagaatgaaaatccgtggctcgcagcgcgcctaccg

SEQ ID NO: 246 - DNA, 162 bp, Artificial Sequence
  source: /note=“Description of Artificial Sequence Synthetic polynucleotide”
gctgcgagccacggacttaaataattgagccttaaagaagaaattctttaagtggatgctctcaaactcagggaaacctaaatctagttatagacaaggcaatcctgagccaagccgaagtagtaattagtaagttaacaacacaaacacaacttatatact

SEQ ID NO: 247 - DNA, 172 bp, Artificial Sequence
  source: /note=“Description of Artificial Sequence Synthetic polynucleotide”
gctgcgagccacggacttaaataattgagccttaaagaagaaattctttaagtggatgctctcaaactcagggaaacctaaatctagttatagacaaggcaatcctgagccaagccgaagtagtaattagtaagttaacaacacaaacacaagagtataagtataatttcag

SEQ ID NO: 248 - DNA, 182 bp, Artificial Sequence
  source: /note=“Description of Artificial Sequence Synthetic polynucleotide”
gctgcgagccacggacttaaataattgagccttaaagaagaaattctttaagtggatgctctcaaactcagggaaacctaaatctagttatagacaaggcaatcctgagccaagccgaagtagtaattagtaagttaacaacacaaacacaagtcatatactgagtataagtataatttcag

SEQ ID NO: 249 - DNA, 192 bp, Artificial Sequence
  source: /note=“Description of Artificial Sequence Synthetic polynucleotide”
gctgcgagccacggacttaaataattgagccttaaagaagaaattctttaagtggatgctctcaaactcagggaaacctaaatctagttatagacaaggcaatcctgagccaagccgaagtagtaattagtaagttaacaacacaaacacaagtcatatactgagtataagtataatttcatattgttgatg

SEQ ID NO: 250 - DNA, 202 bp, Artificial Sequence
  source: /note=“Description of Artificial Sequence Synthetic polynucleotide”
gctgcgagccacggacttaaataattgagccttaaagaagaaattctttaagtggatgctctcaaactcagggaaacctaaatctagttatagacaaggcaatcctgagccaagccgaagtagtaattagtaagttaacaacacaaacacaagcgataatgcttcatatactgagtataagtatagtttcatattgttgatg

SEQ ID NO: 251 - DNA, 162 bp, Artificial Sequence
  source: /note=“Description of Artificial Sequence Synthetic polynucleotide”
gctgcgagccacggacttaaataattgagccttaaagaagaaattctttaagtggatgctctcaaactcagggaaacctaaatctagttatagacaaggcaatcctgagccaagccgaagtagtaattagtaagttaacaaaacaaaaacaagctgacgcta

SEQ ID NO: 252 - DNA, 172 bp, Artificial Sequence
  source: /note=“Description of Artificial Sequence Synthetic polynucleotide”
gctgcgagccacggacttaaataattgagccttaaagaagaaattctttaagtggatgctctcaaactcagggaaacctaaatctagttatagacaaggcaatcctgagccaagccgaagtagtaattagtaagttaacaaaacaaaaacaagctgacgctagtatgagtat

SEQ ID NO: 253 - DNA, 182 bp, Artificial Sequence
  source: /note=“Description of Artificial Sequence Synthetic polynucleotide”
gctgcgagccacggacttaaataattgagccttaaagaagaaattctttaagtggatgctctcaaactcagggaaacctaaatctagttatagacaaggcaatcctgagccaagccgaagtagtaattagtaagttaacaaaacaaaaacaagctgacgctagtatgagtatagcttcacac

SEQ ID NO: 254 - DNA, 192 bp, Artificial Sequence
  source: /note=“Description of Artificial Sequence Synthetic polynucleotide”
gctgcgagccacggacttaaataattgagccttaaagaagaaattctttaagtggatgctctcaaactcagggaaacctaaatctagttatagacaaggcaatcctgagccaagccgaagtagtaattagtaagttaacaaaacaaaaacaagctgacgctagtatgagtatagcttcacactcaggtgagg

SEQ ID NO: 255 - DNA, 202 bp, Artificial Sequence
  source: /note=“Description of Artificial Sequence Synthetic polynucleotide”
gctgcgagccacggacttaaataattgagccttaaagaagaaattctttaagtggatgctctcaaactcagggaaacctaaatctagttatagacaaggcaatcctgagccaagccgaagtagtaattagtaagttaacaaaacaaaaacaagctgacgctagtatgagtatagcttcacactcaggtgaggcatcattcgg

SEQ ID NO: 256 - DNA, 162 bp, Artificial Sequence
  source: /note=“Description of Artificial Sequence Synthetic polynucleotide”
gctgcgagccacggacttaaataattgagccttaaagaagaaattctttaagtggatgctctcaaactcagggaaacctaaatctagttatagacaaggcaatcctgagccaagccgaagtagtaattagtaagttaacaaaacaaaaacaagctcgcaccg

SEQ ID NO: 257 - DNA, 172 bp, Artificial Sequence
  source: /note=“Description of Artificial Sequence Synthetic polynucleotide”
gctgcgagccacggacttaaataattgagccttaaagaagaaattctttaagtggatgctctcaaactcagggaaacctaaatctagttatagacaaggcaatcctgagccaagccgaagtagtaattagtaagttaacaaaacaaaaacaagctcgcactgggtcggagcg

SEQ ID NO: 258 - DNA, 182 bp, Artificial Sequence; identical to SEQ ID NO: 248
  source: /note=“Description of Artificial Sequence Synthetic polynucleotide”

SEQ ID NO: 259 - DNA, 182 bp, Artificial Sequence; identical to SEQ ID NO: 248
  source: /note=“Description of Artificial Sequence Synthetic polynucleotide”

SEQ ID NO: 260 - DNA, 182 bp, Artificial Sequence; identical to SEQ ID NO: 248
  source: /note=“Description of Artificial Sequence Synthetic polynucleotide”

SEQ ID NO: 261 - DNA, 182 bp, Artificial Sequence; identical to SEQ ID NO: 248
  source: /note=“Description of Artificial Sequence Synthetic polynucleotide”

SEQ ID NO: 262 - DNA, 182 bp, Artificial Sequence
  source: /note=“Description of Artificial Sequence Synthetic polynucleotide”
gctgcgagccacggacttaaataattgagccttaaagaagaaattctttaagtggatgctctcaaactcagggaaacctaaatctagttatagacaaggcaatcctgagccaagccgaagtagtaattagtaagttaacaacacaaacacaagtaacttagagagtataagtataatttcag

SEQ ID NO: 263 - DNA, 182 bp, Artificial Sequence
  source: /note=“Description of Artificial Sequence Synthetic polynucleotide”
gctgcgagccacggacttaaataattgagccttaaagaagaaattctttaagtggatgctctcaaactcagggaaacctaaatctagttatagacaaggcaatcctgagccaagccgaagtagtaattagtaagttaacaacacaaacacaagtaacttagatgtaacacacataatttcag

SEQ ID NO: 264 - DNA, 182 bp, Artificial Sequence
  source: /note=“Description of Artificial Sequence Synthetic polynucleotide”
gctgcgagccacggacttaaataattgagccttaaagaagaaattctttaagtggatgctctcaaactcagggaaacctaaatctagttatagacaaggcaatcctgagccaagccgaagtagtaattagtaagttaacaacacaaacacaagtaacttagatgtaacacacaccgatcaac

SEQ ID NO: 265 - DNA, 182 bp, Artificial Sequence
  source: /note=“Description of Artificial Sequence Synthetic polynucleotide”
ctgtttaatcacggacttaaataattgagccttaaagaagaaattctttaagtggatgctctcaaactcagggaaacctaaatctagttatagacaaggcaatcctgagccaagccgaagtagtaattagtaagttaacaacacaaacacaagtcatatactgagtataagtataatttcag

SEQ ID NO: 266 - DNA, 192 bp, Artificial Sequence
  source: /note=“Description of Artificial Sequence Synthetic polynucleotide”
cgtaatttatattgtgaatcacggacttaaataattgagccttaaagaagaaattctttaagtggatgctctcaaactcagggaaacctaaatctagttatagacaaggcaatcctgagccaagccgaagtagtaattagtaagttaacaacacaaacacaagtcatatactgagtataagtataatttcag

SEQ ID NO: 267 - DNA, 182 bp, Artificial Sequence
  source: /note=“Description of Artificial Sequence Synthetic polynucleotide”
gctatgatccacggacttaaataattgagccttaaagaagaaattctttaagtggatgctctcaaactcagggaaacctaaatctagttatagacaaggcaatcctgagccaagccgaagtagtaattagtaagttaacaacacaaacacaagtcatatactgagtataagtataatttcag

SEQ ID NO: 268 - DNA, 192 bp, Artificial Sequence
  source: /note=“Description of Artificial Sequence Synthetic polynucleotide”
cggatattatgctgcgatccacggacttaaataattgagccttaaagaagaaattctttaagtggatgctctcaaactcagggaaacctaaatctagttatagacaaggcaatcctgagccaagccgaagtagtaattagtaagttaacaacacaaacacaagtcatatactgagtataagtataatttcag

SEQ ID NO: 269 - DNA, 192 bp, Artificial Sequence
  source: /note=“Description of Artificial Sequence Synthetic polynucleotide”
cggtaggcgcgctgcgagccacggacttaaataattgagccttaaagaagaaattctttaagtggatgctctcaaactcagggaaacctaaatctagttatagacaaggcaatcctgagccaagccgaagtagtaattagtaagttaacaacacaaacacaagtcatatactgagtataagtataatttcag

SEQ ID NO: 270 - DNA, 215 bp, Artificial Sequence
  source: /note=“Description of Artificial Sequence Synthetic polynucleotide”
gcgctaacgtcgacgccggcaaacggtaggcgcgctgcgagccacggacttaaataattgagccttaaagaagaaattctttaagtggatgctctcaaactcagggaaacctaaatctagttatagacaaggcaatcctgagccaagccgaagtagtaattagtaagttaacaacacaaacacaagtcatatactgagtataagtataatttcag

SEQ ID NO: 271 - DNA, 215 bp, Artificial Sequence
  source: /note=“Description of Artificial Sequence Synthetic polynucleotide”
gcatgtcacttgtatcctcgaaacggatattatgctgcgatccacggacttaaataattgagccttaaagaagaaattctttaagtggatgctctcaaactcagggaaacctaaatctagttatagacaaggcaatcctgagccaagccgaagtagtaattagtaagttaacaacacaaacacaagtcatatactgagtataagtataatttcag

SEQ ID NO: 272 - DNA, 215 bp, Artificial Sequence
  source: /note=“Description of Artificial Sequence Synthetic polynucleotide”
gttagagttatcatttatcgaaacgtaatttagattgtgaatcacggacttaaataattgagccttaaagaagaaattctttaagtggatgctctcaaactcagggaaacctaaatctagttatagacaaggcaatcctgagccaagccgaagtagtaattagtaagttaacaacacaaacacaagtcatatactgagtataagtataatttcag

SEQ ID NO: 273 - DNA, 152 bp, Artificial Sequence
  source: /note=“Description of Artificial Sequence Synthetic polynucleotide”
gctgcgagccacggacttaaataattgagccttaaagaagaaattctttaagtggatgctctcaaactcagggaaacctaaatctagttatagacaaggcaatcctgagccaagccgaagtagtaattagtaagttaacaacacaaacacaa

SEQ ID NO: 274 - DNA, 157 bp, Artificial Sequence
  source: /note=“Description of Artificial Sequence Synthetic polynucleotide”
gctgcgagccacggacttaaataattgagccttaaagaagaaattctttaagtggatgctctcaaactcagggaaacctaaatctagttatagacaaggcaatcctgagccaagccgaagtagtaattagtaagttaacaaaacaaaaacaagcccg

SEQ ID NO: 275 - DNA, 182 bp, Artificial Sequence
  source: /note=“Description of Artificial Sequence Synthetic polynucleotide”
gctgcgagccacggacttaaataattgagccttaaagaagaaattctttaagtggatgctctcaaactcagggaaacctaaatctagttatagacaaggcaatcctgagccaagccgaagtagtaattagtaagttaacaaaacaaaaacaagctcgcactggccggaagctcgtcggagcg

SEQ ID NO: 276 - DNA, 251 bp, Artificial Sequence
  source: /note=“Description of Artificial Sequence Synthetic polynucleotide”
gctgcgagccacggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaatacacaaacacaa

SEQ ID NO: 277 - DNA, 261 bp, Artificial Sequence
  source: /note=“Description of Artificial Sequence Synthetic polynucleotide”
gctgcgagccacggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaatacacaaacacaacttatatact

SEQ ID NO: 278 - DNA, 271 bp, Artificial Sequence
  source: /note=“Description of Artificial Sequence Synthetic polynucleotide”
gctgcgagccacggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaatacacaaacacaagagtataagtataatttcag

SEQ ID NO: 279 - DNA, 281 bp, Artificial Sequence
  source: /note=“Description of Artificial Sequence Synthetic polynucleotide”
gctgcgagccacggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaatacacaaacacaagtcatatactgagtataagtataatttcag

SEQ ID NO: 280 - DNA, 261 bp, Artificial Sequence
  source: /note=“Description of Artificial Sequence Synthetic polynucleotide”
gctgcgagccacggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaataaacaaaaacaagctgacgcta

SEQ ID NO: 281 - DNA, 271 bp, Artificial Sequence
  source: /note=“Description of Artificial Sequence Synthetic polynucleotide”
gctgcgagccacggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaataaacaaaaacaagctgacgctagtatgagtat

SEQ ID NO: 282 - DNA, 281 bp, Artificial Sequence
  source: /note=“Description of Artificial Sequence Synthetic polynucleotide”
gctgcgagccacggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaataaacaaaaacaagctgacgctagtatgagtatagcttcacac

SEQ ID NO: 283 - DNA, 261 bp, Artificial Sequence
  source: /note=“Description of Artificial Sequence Synthetic polynucleotide”
gctgcgagccacggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaataaacaaaaacaagctcgcaccg

SEQ ID NO: 284 - DNA, 271 bp, Artificial Sequence
  source: /note=“Description of Artificial Sequence Synthetic polynucleotide”
gctgcgagccacggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaataaacaaaaacaagctcgcactgggtcggagcg

SEQ ID NO: 285 - DNA, 281 bp, Artificial Sequence
  source: /note=“Description of Artificial Sequence Synthetic polynucleotide”
gctgcgagccacggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaataaacaaaaacaagctcgcactggccggaagctcgtcggagcg

SEQ ID NO: 286 - DNA, 210 bp, Artificial Sequence
  source: /note=“Description of Artificial Sequence Synthetic polynucleotide”
gctgcgagccacggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtccacaaacacaa

SEQ ID NO: 287 - DNA, 220 bp, Artificial Sequence
  source: /note=“Description of Artificial Sequence Synthetic polynucleotide”
gctgcgagccacggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtccacaaacacaacttatatact

SEQ ID NO: 288 - DNA, 230 bp, Artificial Sequence
  source: /note=“Description of Artificial Sequence Synthetic polynucleotide”
gctgcgagccacggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtccacaaacacaagagtataagtataatttcag

SEQ ID NO: 289 - DNA, 240 bp, Artificial Sequence
  source: /note=“Description of Artificial Sequence Synthetic polynucleotide”
gctgcgagccacggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtccacaaacacaagtcatatactgagtataagtataatttcag

SEQ ID NO: 290 - DNA, 220 bp, Artificial Sequence
  source: /note=“Description of Artificial Sequence Synthetic polynucleotide”
gctgcgagccacggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaacaaaaacaagctgacgcta

SEQ ID NO: 291 - DNA, 230 bp, Artificial Sequence
  source: /note=“Description of Artificial Sequence Synthetic polynucleotide”
gctgcgagccacggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaacaaaaacaagctgacgctagtatgagtat

SEQ ID NO: 292 - DNA, 240 bp, Artificial Sequence
  source: /note=“Description of Artificial Sequence Synthetic polynucleotide”
gctgcgagccacggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaacaaaaacaagctgacgctagtatgagtatagcttcacac

SEQ ID NO: 293 - DNA, 220 bp, Artificial Sequence
  source: /note=“Description of Artificial Sequence Synthetic polynucleotide”
gctgcgagccacggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaacaaaaacaagctcgcaccg

SEQ ID NO: 294 - DNA, 230 bp, Artificial Sequence
  source: /note=“Description of Artificial Sequence Synthetic polynucleotide”
gctgcgagccacggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaacaaaaacaagctcgcactgggtcggagcg

SEQ ID NO: 295 - DNA, 240 bp, Artificial Sequence
  source: /note=“Description of Artificial Sequence Synthetic polynucleotide”
gctgcgagccacggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaacaaaaacaagctcgcactggccggaagctcgtcggagcg

SEQ ID NO: 296 - DNA, 172 bp, Artificial Sequence
  source: /note=“Description of Artificial Sequence Synthetic polynucleotide”
acggacttaaataattgagccttaaagaagaaattctttaagtggatgctctcaaactcagggaaacctaaatctagttatagacaaggcaatcctgagccaagccgaagtagtaattagtaagttaacaacacaaacacaagtcatatactgagtataagtataatttcag

SEQ ID NO: 297 - DNA, 271 bp, Artificial Sequence
  source: /note=“Description of Artificial Sequence Synthetic polynucleotide”
acggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaatacacaaacacaagtcatatactgagtataagtataatttcag

SEQ ID NO: 298 - DNA, 281 bp, Artificial Sequence
  source: /note=“Description of Artificial Sequence Synthetic polynucleotide”
ctgtttaatcacggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaatacacaaacacaagtcatatactgagtataagtataatttcag

SEQ ID NO: 299 - DNA, 291 bp, Artificial Sequence
  source: /note=“Description of Artificial Sequence Synthetic polynucleotide”
cgtaatttatattgtgaatcacggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaatacacaaacacaagtcatatactgagtataagtataatttcag

SEQ ID NO: 300 - DNA, 281 bp, Artificial Sequence
  source: /note=“Description of Artificial Sequence Synthetic polynucleotide”
gctatgatccacggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaatacacaaacacaagtcatatactgagtataagtataatttcag

SEQ ID NO: 301 - DNA, 291 bp, Artificial Sequence
  source: /note=“Description of Artificial Sequence Synthetic polynucleotide”
cggatattatgctgcgatccacggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaatacacaaacacaagtcatatactgagtataagtataatttcag

SEQ ID NO: 302 - DNA, 291 bp, Artificial Sequence
  source: /note=“Description of Artificial Sequence Synthetic polynucleotide”
cggtaggcgcgctgcgagccacggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaatacacaaacacaagtcatatactgagtataagtataatttcag

SEQ ID NO: 303 - DNA, 230 bp, Artificial Sequence
  source: /note=“Description of Artificial Sequence Synthetic polynucleotide”
acggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtccacaaacacaagtcatatactgagtataagtataatttcag

SEQ ID NO: 304 - DNA, 240 bp, Artificial Sequence
  source: /note=“Description of Artificial Sequence Synthetic polynucleotide”
ctgtttaatcacggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtccacaaacacaagtcatatactgagtataagtataatttcag

SEQ ID NO: 305 - DNA, 250 bp, Artificial Sequence
  source: /note=“Description of Artificial Sequence Synthetic polynucleotide”
cgtaatttatattgtgaatcacggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtccacaaacacaagtcatatactgagtataagtataatttcag

SEQ ID NO: 306 - DNA, 240 bp, Artificial Sequence
  source: /note=“Description of Artificial Sequence Synthetic polynucleotide”
gctatgatccacggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtccacaaacacaagtcatatactgagtataagtataatttcag

SEQ ID NO: 307 - DNA, 250 bp, Artificial Sequence
  source: /note=“Description of Artificial Sequence Synthetic polynucleotide”
cggatattatgctgcgatccacggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtccacaaacacaagtcatatactgagtataagtataatttcag

SEQ ID NO: 308 - DNA, 250 bp, Artificial Sequence
  source: /note=“Description of Artificial Sequence Synthetic polynucleotide”
cggtaggcgcgctgcgagccacggacttaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtccacaaacacaagtcatatactgagtataagtataatttcag

SEQ ID NO: 309 - protein, 20 aa, Artificial Sequence
  source: /note=“Description of Artificial Sequence Synthetic peptide”
YHADYYKQRLIHDVEMNPGP

SEQ ID NO: 310 - protein, 20 aa, Artificial Sequence
  source: /note=“Description of Artificial Sequence Synthetic peptide”
HYAGYFADLLIHDIETNPGP

SEQ ID NO: 311 - protein, 20 aa, Artificial Sequence
  source: /note=“Description of Artificial Sequence Synthetic peptide”
QCTNYALLKLAGDVESNPGP

SEQ ID NO: 312 - protein, 19 aa, Artificial Sequence
  source: /note=“Description of Artificial Sequence Synthetic peptide”
ATNFSLLKQAGDVEENPGP

SEQ ID NO: 313 - protein, 19 aa, Artificial Sequence
  source: /note=“Description of Artificial Sequence Synthetic peptide”
AARQMLLLLSGDVETNPGP

SEQ ID NO: 314 - protein, 20 aa, Artificial Sequence
  source: /note=“Description of Artificial Sequence Synthetic peptide”
RAEGRGSLLTCGDVEENPGP

SEQ ID NO: 315 - protein, 20 aa, Artificial Sequence
  source: /note=“Description of Artificial Sequence Synthetic peptide”
TRAEIEDELIRAGIESNPGP

SEQ ID NO: 316 - protein, 20 aa, Artificial Sequence
  source: /note=“Description of Artificial Sequence Synthetic peptide”
AKFQIDKILISGDVELNPGP

SEQ ID NO: 317 - protein, 20 aa, Artificial Sequence
  source: /note=“Description of Artificial Sequence Synthetic peptide”
SSIIRTKMLVSGDVEENPGP

SEQ ID NO: 318 - protein, 20 aa, Artificial Sequence
  source: /note=“Description of Artificial Sequence Synthetic peptide”
CDAQRQKLLLSGDIEQNPGP

SEQ ID NO: 319 - protein, 20 aa, Artificial Sequence
  source: /note=“Description of Artificial Sequence Synthetic peptide”
YPIDFGGFLVKADSEFNPGP

SEQ ID NO: 320 - protein, 22 aa, Artificial Sequence
  source: /note=“Description of Artificial Sequence Synthetic peptide”
GSGATNFSLLKQAGDVEENPGP

SEQ ID NO: 321 - protein, 21 aa, Artificial Sequence
  source: /note=“Description of Artificial Sequence Synthetic peptide”
GSGEGRGSLLTCGDVEENPGP

SEQ ID NO: 322 - protein, 23 aa, Artificial Sequence
  source: /note=“Description of Artificial Sequence Synthetic peptide”
GSGQCTNYALLKLAGDVESNPGP

SEQ ID NO: 323 - protein, 25 aa, Artificial Sequence
  source: /note=“Description of Artificial Sequence Synthetic peptide”
GSGVKQTLNFDLLKLAGDVESNPGP

SEQ ID NO: 324 - protein, 9 aa, Artificial Sequence
  source: /note=“Description of Artificial Sequence Synthetic peptide”
  VARIANT (5)..(5): /replace=“Ser”
  SITE (1)..(9): /note=“Variant residues given in the sequence have no preference with respect to those in the annotations for variant positions”
GDVEENPGP

SEQ ID NO: 325 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 326 - protein, 7096 aa, Severe acute respiratory syndrome coronavirus 2 [sequence omitted]

SEQ ID NO: 327 - protein, 4405 aa, Severe acute respiratory syndrome coronavirus 2 [sequence omitted]

SEQ ID NO: 328 - protein, 275 aa, Severe acute respiratory syndrome coronavirus 2
MDLFMRIFTIGTVTLKQGEIKDATPSDFVRATATIPIQASLPFGWLIVGVALLAVFQSASKIITLKKRWQLALSKGVHFVCNLLLLFVTVYSHLLLVAAGLEAPFLYLYALVYFLQSINFVRIIMRLWLCWKCRSKNPLLYDANYFLCWHTNCYDYCIPYNSVTSSIVITSGDGTTSPISEHDYQIGGYTEKWESGVKDCVVLHSYFTSDYYQLYSTQLSTDTGVEHVTFFIYNKIVDEPEEHVQIHTIDGSSGVVNPVMEPIYDEPTTTTSVPL

SEQ ID NO: 329 - protein, 75 aa, Severe acute respiratory syndrome coronavirus 2
MYSFVSEETGTLIVNSVLLFLAFVVFLLVTLAILTALRLCAYCCNIVNVSLVKPSFYVYSRVKNLNSSRVPDLLV

SEQ ID NO: 330 - protein, 222 aa, Severe acute respiratory syndrome coronavirus 2
MADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 331 - protein, 61 aa, Severe acute respiratory syndrome coronavirus 2
MFHLVDFQVTIAEILLIIMRTFKVSIWNLDYIINLIIKNLSKSLTENKYSQLDEEQPMEID

SEQ ID NO: 332 - protein, 121 aa, Severe acute respiratory syndrome coronavirus 2
MKIILFLALITLATCELYHYQECVRGTTVLLKEPCSSGTYEGNSPFHPLADNKFALTCFSTQFAFACPDGVKHVYQLRARSVSPKLFIRQEEVQELYSPIFLIVAAIVFITLCFTLKRKTE

SEQ ID NO: 333 - protein, 43 aa, Severe acute respiratory syndrome coronavirus 2
MIELSLIDFYLCFLAFLLFLVLIMLIIFWFSLELQDHNETCHA

SEQ ID NO: 334 - protein, 121 aa, Severe acute respiratory syndrome coronavirus 2
MKFLVFLGIITTVAAFHQECSLQSCTQHQPYVVDDPCPIHFYSKWYIRVGARKSAPLIELCVDEAGSKSPIQYIDIGNYTVSCLPFTINCQEPKLGSLVVRCSFYEDFLEYHDVRVVLDFI

SEQ ID NO: 335 - protein, 419 aa, Severe acute respiratory syndrome coronavirus 2
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 336 - protein, 38 aa, Severe acute respiratory syndrome coronavirus 2
MGYINVFAFPFTIYSLLLCRMNSRNYIAQVDVVNFNLT

SEQ ID NO: 337 - protein, 329 aa, Unknown
  source: /note=“Description of Unknown BCSP31 sequence”
MKFGSKIRRLAVAAVAGAIALGASFAVAQAPTFFRIGTGGTAGTYYPIGGLIANAISGAGEKGVPGLVATAVSSNGSVANINAIKSGALESGFTQSDVAYWAYNGTGLYDGKGKVEDLRLLATLYPETIHIVARKDANIKSVADLKGKRVSLDEPGSGTIVDARIVLEAYGLTEDDIKAEHLKPGPAGERLKDGALDAYFFVGGYPTGAISELAISNGISLVPISGPEADKILEKYSFFSKDVVPAGAYKDVAETPTLAVAAQWVTSAKQPDDLIYNITKVLWNEDTRKALDAGHAKGKLIKLDSATSSLGIPLHPGAERFYKEAGVLK

SEQ ID NO: 338 - protein, 402 aa, Unknown
  source: /note=“Description of Unknown MOMP sequence”
MKKLLKSALLFAATGSALSLQALPVGNPAEPSLLIDGTMWEGASGDPCDPCATWCDAISIRAGYYGDYVFDRVLKVDVNKTFSGMAATPTQATGNASNTNQPEANGRPNIAYGRHMQDAEWFSNAAFLALNIWDRFDIFCTLGASNGYFKASSAAFNLVGLIGFSAASSISTDLPMQLPNVGITQGVVEFYTDTSFSWSVGARGALWECGCATLGAEFQYAQSNPKIEMLNVTSSPAQFVIHKPRGYKGASSNFPLPITAGTTEATDTKSATIKYHEWQVGLALSYRLNMLVPYIGVNWSRATFDADTIRIAQPKLKSEILNITTWNPSLIGSTTALPNNSGKDVLSDVLQIASIQINKMKSRKACGVAVGATLIDADKWSITGEARLINERAAHMNAQFRF

SEQ ID NO: 339 - protein, 498 aa, Unknown
  source: /note=“Description of Unknown Flagellin sequence”
MAQVINTNSLSLITQNNINKNQSALSSSIERLSSGLRINSAKDDAAGQAIANRFTSNIKGLTQAARNANDGISVAQTTEGALSEINNNLQRVRELTVQATTGTNSESDLSSIQDEIKSRLDEIDRVSGQTQFNGVNVLAKNGSMKIQVGANDNQTITIDLKQIDAKTLGLDGFSVKNNDTVTTSAPVTAFGATTTNNIKLTGITLSTEAATDTGGTNPASIEGVYTDNGNDYYAKITGGDNDGKYYAVTVANDGTVTMATGATANATVTDANTTKATTITSGGTPVQIDNTAGSATANLGAVSLVKLQDSKGNDTDTYALKDTNGNLYAADVNETTGAVSVKTITYTDSSGAASSPTAVKLGGDDGKTEVVDIDGKTYDSADLNGGNLQTGLTAGGEALTAVANGKTTDPLKALDDAIASVDKFRSSLGAVQNRLDSAVTNLNNTTTNLSEAQSRIQDADYATEVSNMSKAQIIQQAGNSVLAKANQVPQQVLSLLQG

SEQ ID NO: 340 - protein, 189 aa, Homo sapiens
MASPFALLMVLVVLSCKSSCSLGCDLPETHSLDNRRTLMLLAQMSRISPSSCLMDRHDFGFPQEEFDGNQFQKAPAISVLHELIQQIFNLFTTKDSSAAWDEDLLDKFCTELYQQLNDLEACVMQEERVGETPLMNADSILAVKKYFRRITLYLTEKKYSPCAWEVVRAEIMRSLSLSTNLQERLRRKE

SEQ ID NO: 341 - protein, 166 aa, Homo sapiens
MKYTSYILAFQLCIVLGSLGCYCQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 342 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 343 - protein, 162 aa, Homo sapiens
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 344 - protein, 193 aa, Homo sapiens
MAAEPVEDNCINFVAMKFIDNTLYFIAEDDENLESDYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 345 - protein, 235 aa, Unknown
  source: /note=“Description of Unknown FLt3-ligand sequence”
MTVLAPAWSPTTYLLLLLLLSSGLSGTQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQPPLLLLLLLPVGLLLLAAAWCLHWQRTRRRTPRPGEQVPPVPSPQDLLLVEH

SEQ ID NO: 346 - protein, 448 aa, Artificial Sequence
  source: /note=“Description of Artificial Sequence Synthetic polypeptide”
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 347 - protein, 440 aa, Artificial Sequence
  source: /note=“Description of Artificial Sequence Synthetic polypeptide”
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 348 - protein, 442 aa, Artificial Sequence
  source: /note=“Description of Artificial Sequence Synthetic polypeptide”
EVQLVQSGAEVKKPGESLRISCKGSGYSFSTYWISWVRQMPGKGLEWMGKIYPGDSYTNYSPSFQGQVTISADKSISTAYLQWSSLKASDTAMYYCARGYGIFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 349 - protein, 5 aa, Artificial Sequence
  source: /note=“Description of Artificial Sequence Synthetic peptide”
GGGGS

SEQ ID NO: 350 - protein, 15 aa, Artificial Sequence
  source: /note=“Description of Artificial Sequence Synthetic peptide”
GGGGSGGGGSGGGGS

SEQ ID NO: 351 - DNA, 30 bp, Artificial Sequence
  source: /note=“Description of Artificial Sequence Synthetic oligonucleotide”
tataattctaccctattgaggcattgacta